(19) United States
(12) Patent Application Publication
(10) Pub. No.: US 2022/0348558 A1
(43) Pub. Date: Nov. 3, 2022

(54) INHIBITORS OF ENCEPHALITIC ALPHAVIRUSES

(71) Applicant: Wisconsin Alumni Research Foundation, Madison, WI (US)

(72) Inventors: Jennifer Elizabeth Golden, Waunakee, WI (US); Xufeng Cao, West Lafayette, WI (US); Donghoon Chung, Prospect, KY (US); Colleen Beth Jonsson, Knoxville, TN (US); Xiaoyu Li, Madison, WI (US); Michael Ryan, Madison, WI (US); Jongwoo Son, Saha-gu, Busan (KR)

(21) Appl. No.: 17/639,615

(22) PCT Filed: Sep. 4, 2020
(86) PCT No.: PCT/US2020/049351

§ 371 Date: Mar. 2, 2022

Related U.S. Application Data
(60) Provisional application No. 62/896,081, filed on Sep. 5, 2019.

(51) Int. Cl. C07D 403/04; C07D 405/14; C07D 401/14; C07D 403/14; C07D 487/08; C07D 491/107; C07D 413/14; C07D 239/90; A61P 31/14

(52) U.S. Cl. CPC C07D 403/04; C07D 405/14; C07D 401/14; C07D 403/14; C07D 487/08; C07D 491/107; C07D 413/14; C07D 239/90; A61P 31/14

(57) ABSTRACT

Compounds of Formula I and Formula II:

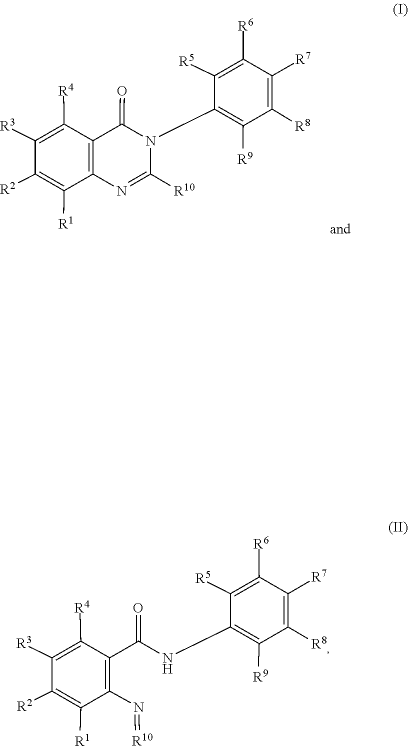

pharmaceutical compositions containing them, and use of the compounds as active ingredients to treat infection with alphavirus.

### CROSS-REFERENCE TO RELATED APPLICATIONS

Priority is hereby claimed to PCT Application Ser. No. PCT/US2020/049351, filed Sep. 4, 2020, which claims priority to provisional application Ser. No. 62/896,081, filed Sep. 5, 2019, which is incorporated herein by reference.

### FEDERAL FUNDING STATEMENT

This invention was made with government support under AI118814 and AI142792 awarded by the National Institutes of Health. The government has certain rights in the invention.

### BACKGROUND

The alphaviruses are small, spherical, enveloped viruses with a genome of a single-sense, positive-strand RNA genome. The alphavirus species have been categorized into “Old World” and “New World” classes based on differences in the proteins involved in inducing transcriptional shutoff. See Garmashova et al., J. Virol. March 2007, 81(5):2472-2484. Included among the “New World” alphaviruses are several species that are pathogenic in vertebrates, including humans. Among these, the most economically significant are Venezuelan equine encephalitis virus (“VEEV”), eastern equine encephalitis virus (“EEEV”) and western equine encephalitis virus (“WEEV”). As the names indicate, these pathogenic viruses cause encephalitis in horses. The virus can spread to humans, typically via mosquitos and other arthropods.

Most alphaviruses are maintained in natural cycles between the arthropod vectors (mainly mosquitoes) and susceptible vertebrate hosts. See Strauss and Strauss, “The alphaviruses: gene expression, replication, and evolution,” Microbiol. Rev. 1994, 58(3): 491-562. These cycles periodically flow into human and animal populations and cause outbreaks of disease. Human infections with Old World alphaviruses such as Ross River (“RRV”), chikungunya (“CHIKV”), and Sindbis (“SINV”) viruses are characterized by febrile illness, rash and polyarthritis. In contrast, infections with any of the New World alphaviruses VEEV, EEEV, or Western equine encephalitis can cause fatal encephalitis in both humans and non-human vertebrates (horses being the most economically significant non-human victims). The ability of alphaviruses to infect both invertebrates and vertebrates facilitates a broad host range that enables the viruses to be maintained in ecological niches with sporadic outbreaks in humans and animals.

Alphaviruses infect many vertebrate and invertebrate hosts, including at least three genera of mosquitos (Aedes sp., Culex sp., and Anopheles sp.), ticks, and lice. Vertebrate hosts include fish, equids, birds, amphibians, reptiles, rodents, pigs, humans and non-human primates. See Griffin. “Alphaviruses,” In: Fields B N, Knipe D M, Howley P M, editors, Virology, 5th edition, ISBN 978-0781760607, © 2006, Lippincott Williams & Wilkins, New York, N.Y., pp. 1023-68; Linthicum et al., J Med. Entomol. 1991, 28(3):405-9; and La Linn et al., J Virol. 2001, 75(9):4103-9). As a consequence, alphaviruses are readily cultured in vitro in many vertebrate and invertebrate cell lines. See, for example, Way et al., J Gen Virol. 1976, 30(1):123-30; Sarver and Stollar, Virology 1977, 80(2):390-400; and Igarashi, J Gen Virol. 1978, 40(3):531-44.

### SUMMARY

Disclosed herein are compounds of Formula I and Formula II:

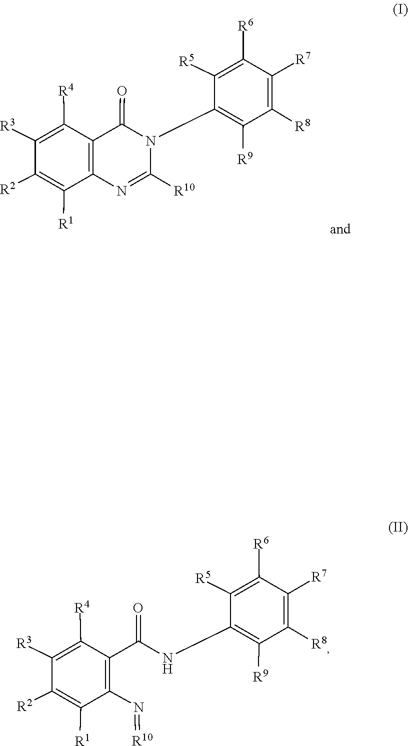

wherein:

R¹, R², R³ and R⁴ are the same or different and are independently selected from hydrogen, halogen, alkyl, C₃-C₈-cycloalkyl, halogen-substituted alkyl, alkoxy, halogen-substituted alkoxy, nitro, cyano, aryl, alkyl-substituted aryl, alkoxy-substituted aryl, halogen-substituted aryl, saturated or unsaturated 4- to 6-membered heterocyclyl, sulfonyl, and sulfonamidyl,

provided that not all of R¹, R², R³ and R⁴ are simultaneously hydrogen, and

provided that at least one of R¹, R², R³ and R⁴ is an electron-withdrawing group;

R⁵, R⁶, R⁷, R⁸, and R⁹ are the same or different and are independently selected from hydrogen, halogen, alkyl, unsubstituted or substituted C₃-C₈-cycloalkyl, halogen-substituted alkyl, alkoxy, halogen-substituted alkoxy, azido, nitro, and cyano;

R¹⁰ is selected from aliphatic amino, aliphatic alkylamino,

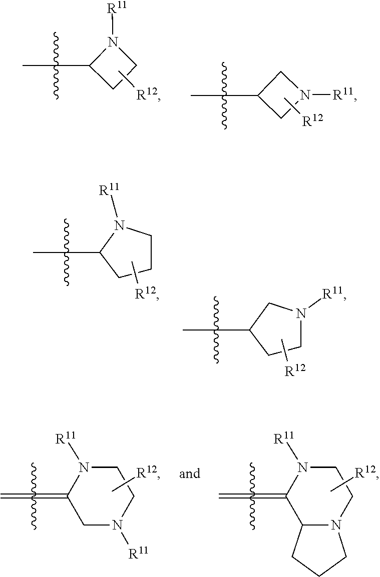

wherein:

R¹¹ is selected from hydrogen, halogen, alkyl, halogen-substituted alkyl, alkoxy, halogen-substituted alkoxy, unsubstituted or substituted C₃-C₈-cyclic ether, and unsubstituted or C-substituted or N-substituted saturated or unsaturated nitrogen C₄-C₈ heterocyclyl;

R¹² is selected from hydrogen, halogen, alkyl, halogen-substituted alkyl, alkoxy, and halogen-substituted alkoxy;

and salts thereof.

In preferred versions of the compounds, R¹⁰ is selected from

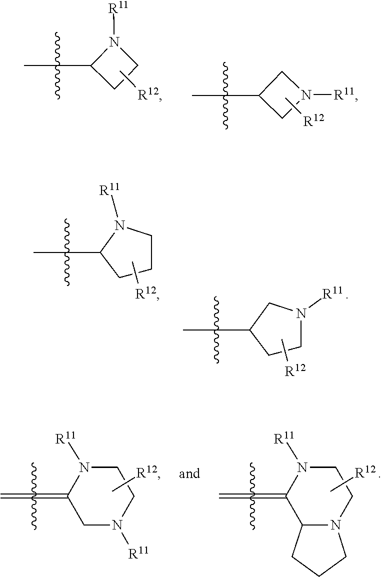

R¹¹ may optionally be selected from hydrogen, halogen, and alkyl.

R¹² may optionally be selected from hydrogen, halogen, and alkyl.

In other versions of the compounds, R¹¹ is selected from unsubstituted or substituted C₃-C₆-cyclic ether, and unsubstituted or C-substituted or N-substituted C₄-C₆-saturated nitrogen heterocyclyl.

R³ may optionally be selected from selected from fluoro, nitro, cyano, sulfonyl, and sulfonamide.

In another version of the compounds, R¹ is not hydrogen.

In all versions of the compounds, R¹ may optionally be selected from halogen, alkyl, halogen-substituted alkyl, C₃-C₈-cycloalkyl, 3-pyrimidinyl, and 4-pyrimidinyl.

The compounds may have a structure as shown in Formula I, with R¹⁰ being optionally selected from

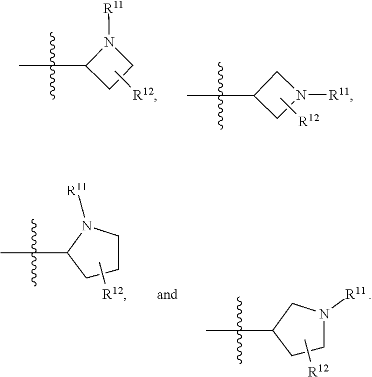

Or, the compounds may have a structure as shown in Formula II, with R¹⁰ being optionally selected from

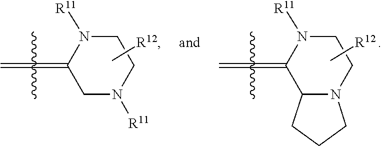

Also disclosed herein is a pharmaceutical composition for inhibiting a viral infection. The composition comprises an effective amount of one or more compounds as disclosed herein.

Further disclosed herein is a method of inhibiting a viral infection. The method comprises administering to a subject an antiviral-effective amount of a compound as disclosed herein.

### BRIEF DESCRIPTION OF THE DRAWING

The sole FIGURE is a graph depicting the results of a fourteen (14)-day in vivo efficacy assessment of Example 55, compound 7a, in 6 to 8 week-old BALB/C mice (groups of 8). The study was a prophylactic assessment: compound was given to mice 2 h prior to viral exposure with VEEV TrD (10×LD50, subcutaneous challenge). Compound was delivered via IP injection. Per-day dose was divided into two doses, given 12 h apart for 8 days (e.g. 12 mg/kg/day was given as 6 mg/kg BID). Virus+vehicle mice all died without compound treatment by Day 5. Mice without virus all survived (control, 100% survival). 48 mg/kg/day group had a 100% survival rate. Mice in the 24 mg/kg/day and 12 mg/kg/day groups had 75% survival rates. Survival plot lines are offset for clarity.

### DETAILED DESCRIPTION

In various aspects, the present technology provides novel compounds and methods for treating a viral infection. The compounds provided herein can be formulated into pharmaceutical compositions and medicaments that are useful for inhibiting infection with alphaviruses. Also provided is the use of the compounds in preparing pharmaceutical formulations and medicaments, the use of the compounds in treating a viral infection in general and an alpha virus infection in particular.

### Definitions

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., Cl, F, Br, and I); hydroxyls; alkoxy, alkenoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-, 2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, CH═CH(CH₃), CH═C(CH₃)₂, C(CH₃)═CH₂, C(CH₃)═CH(CH₃), C(CH₂CH₃)═CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithionyl, dihydrodithionyl, homopiperazinyl, quinuclidyl, indolyl, indolinyl, isoindolyl, azaindolyl(pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl(azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl(pyrrolopyridinyl), indazolyl, benzimidazolyl, imidazopyridinyl(azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) bonded to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(═O)-alkyl groups and —O—C(═O)-alkyl groups, each containing 2-5 carbon atoms.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR groups, in which R is a substituted or unsubstituted alkyl, cycloalkyl, alkenyl, alkynyl, aryl, aralkyl, heterocyclylalkyl or heterocyclyl group as defined herein.

The term “amide” (or “amido”) includes C- and N-amide groups, i.e., C(═O)NRR′, and —NRC(═O)R′ groups, respectively. R and R′ are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(═O)NH₂) and formamide groups (—NHC(═O)H). In some embodiments, the amide is —NRC(═O)—(C₁₋₅ alkyl) and the group is termed “carbonylamino,” and in others the amide is —NHC(═O)-alkyl and the group is termed “alkanoylamino.”

The term “nitrile” or “cyano” as used herein refers to the —C≡N group.

Urethane groups include N- and O-urethane groups, i.e., —NRC(═O)OR′ and —OC(═O)NRR′ groups, respectively. R and R′ are independently a substituted or unsubstituted alkyl, alkenyl, alkynyl, cycloalkyl, aryl, aralkyl, heterocyclylalkyl, or heterocyclyl group as defined herein. R or R′ may also be H.

The term “amine” (or “amino”) as used herein refers to —NRR′ groups, wherein R and R′ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NRR′ and NRSO₂R′ groups, respectively. Rand R′ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while sulfides include —SR groups, sulfoxides include —S(═O)R groups, sulfones include —SO₂R groups, and sulfonyls include —SO₂OR. Each R group each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR—C(═O)—NR′R″ groups. R, R′, and R″ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR)NR′R″ and —NRC(NR′)R″, wherein R, R′, and R″ are each independently hydrogen, or a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to chlorine, fluorine, bromine, and iodine. In some embodiments, the halogen is fluorine. In other embodiments, the halogen is chlorine or bromine.

The term “hydroxy” as used herein can refer to —OH or its ionized form, —O⁻.

The term “nitro” as used herein refers to an —NO₂ group.

The term “trifluoromethyl” as used herein refers to —CF₃.

The term “trifluoromethoxy” as used herein refers to —OCF₃.

The phrase “selectively treats” as used herein will be understood by persons of ordinary skill in the art and will vary to some extent depending upon the context in which the phrase is used. If there are uses of the phrase which are not clear to persons of ordinary skill in the art, given the context in which the phrase is used, the phrase at minimum refers to the compounds acting through a viral-specific mechanism of action, resulting in fewer off-target effects because the compounds target the virus and not the host.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g. alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g. Na⁺, Li⁺, K⁺, Ca²⁺, Mg²⁺, zn²⁺), ammonia or organic amines (e.g. dicyclohexylamine, trimethylamine, triethylamine, pyridine, picoline, ethanolamine, diethanolamine, triethanolamine) or basic amino acids (e.g. arginine, lysine and ornithine). Such salts can be prepared in situ during isolation and purification of the compounds or by separately reacting the purified compound in its free base or free acid form with a suitable acid or base, respectively, and isolating the salt thus formed. See, for example, See, for example, “Handbook of Pharmaceutical Salts, Properties, Selection, and Use,” P. H. Stahl and C. G. Wermuch, Eds., © 2008, Wiley-VCH (Zurich, Switzerland), ISBN: 978-3-90639-058-1.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

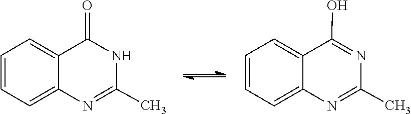

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions (including enantiomers, diasteromers, and atropisomers). Racemic and diastereomeric mixtures, as well as the individual optical isomers can be enriched in any proportion or isolated or synthesized so as to be substantially free of their enantiomeric or diastereomeric partners, and these stereoisomers and atropisomers are all within the scope of the present disclosure.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

“Effective amount” refers to the amount of a compound or composition required to produce a desired effect. One example of an effective amount includes amounts or dosages that yield acceptable toxicity and bioavailability levels for therapeutic (pharmaceutical) use including, but not limited to, the treatment or prophylaxis of an encephalitic alphavirus. Another example of an effective amount includes amounts or dosages that are capable of reducing symptoms associated with an encephalitic alphavirus, such as, for example, fever, headache, and encephalitis.

An “electron-withdrawing group” is an atom or functional group attached to an aromatic or heteroaromatic ring, and which withdraws electron density from the conjugated π-electrons in the ring to which it is attached. Electron-withdrawing groups can withdraw π-electron density by inductive effects, resonance effects, or both. A non-limiting list of examples of electron-withdrawing groups includes trifluoromethylsulfonyl (—SO₂CF₃), ammonium groups (—NR₃⁺ (R=alkyl or H)), nitro (—NO₂), sulfonic acids and sulfonyl groups (—SO₃H, —SO₂R), cyano (—C≡N), trihalomethyl (—CX₃ (X═F, Cl, Br, I)), haloformyl groups (—COX (X═Cl, Br, I)), formyl and acyl groups (—C(═O)H, —C(═O)R), carboxyl and alkoxycarbonyl groups (—C(═O)OH, —C(═O)O—R), aminocarbonyl groups (—C(═O)NH₂, —C(═O)NHR, —C(═O)NR₂), halo, nitroso (—N═O), and the like.

As used herein, a “subject” or “patient” is a mammal, such as a cat, dog, rodent, horse, or primate. Typically the subject is a human, and, preferably, a human suspected of having a disease associated with an alphavirus. The term “subject” and “patient” can be used interchangeably.

### INTRODUCTION

VEEV, a member of family Togaviridae, may result in a severe neurological disease in approximately 14 percent of humans and horses infected. Epidemics have occurred affecting hundreds of thousands of people in the Americas for nearly a century. An estimated 70,000 to 100,000 humans and similar numbers of horses infected with VEEV were reported during the last outbreak in 1995. The disease in humans is characterized by fever, headache, and encephalitis to varying degrees and is sometimes fatal. The mortality rate is below 1%; however, the neurological disease is present in up to 14% of patients. The virus is usually transmitted via mosquito bite, but evidence supports viral transmission by aerosol via man-made technologies and hence this virus is considered a biothreat. The modes of transmission make VEEV infection very difficult to control during outbreaks. Thus, prophylaxis and efficacious treatments are critical to minimizing the impact of the transmissible disease on human and equines.

Currently, there are no FDA-approved vaccines or therapeutics for the encephalitic alphaviruses, which limit treatment to supportive care. The US Army has been developing vaccines for VEEV as they appreciate the impact of the disease on soldiers as well as its potential use as a bioweapon. The vaccines, which are comprised of attenuated live virus, are still in the investigational new drug (IND) stage and are only available through the Special Immunization Program at United State Army Medical Research Institute of Infectious Diseases (USAMRIID) for protecting personnel working with the virus. A few other vaccine candidates are in the IND stage, such as formalized killed TC-83 vaccine and the live attenuated V3526 vaccine. Again those vaccines have not been FDA-approved due to lack of efficacy and adverse effects seen during clinical trials.

The alphaviruses, of which VEEV is a member, include other medically important viruses such as Eastern and Western equine encephalitis viruses (EEEV and WEEV respectively), Sindbis virus (SINV) and Chikungunya (CHIK) viruses. VEE, EEE and CHIK viruses are listed as Center for Disease Control (CDC) category B and C agents because of their potential military and bioterrorism threats. They are relatively easy to produce at high titers, are highly infectious by aerosol, and can cause severely debilitating disease and death. Finally, VEEV, eastern (EEEV), western (WEEV) and Chikungunya (CHIK) viruses are closely related phylogentically. Therefore, discovery of VEEV inhibitors would be equally effective against closely related other encephalitic alphaviruses.

### The Compounds:

The present technology is directed to compounds, compositions, and methods to treat an alphavirus infection. The technology is especially suited to treat an encephalitic alphavirus. The compounds and compositions described herein may be used in the treatment or prophylaxis of diseases that include, for example, Venezuelan equine encephalitis virus (VEEV). Methods of treatment include administering to a subject in need thereof a therapeutically effective amount of a compound or composition described herein. The compounds of the present technology can also be used in the treatment or prophylaxis of a disease state or malady characterized by or associated with an alphavirus. Generally, prophylactic or prophylaxis relates to a reduction in the likelihood of the patient developing a disorder such as VEEV infection or proceeding to a diagnosis state for the disorder. For example, the compounds of the present technology can be used prophylactically as a measure designed to preserve health and prevent the spread or maturation of disease in a patient. It is also appreciated that the various modes of treatment or prevention of a disease such as an alphavirus infection can mean “substantial” treatment or prevention, which includes total but also less than total treatment or prevention, and in which some biologically or medically relevant result is achieved. Furthermore, treatment or treating as well as alleviating can refer to therapeutic treatment and prophylactic or preventative measures in which the objective is to prevent, slow down (lessen) a disease state, condition or malady. For example, a subject can be successfully treated for an alphavirus infection if, after receiving through administration an effective or therapeutic amount of one or more compounds described herein, the subject shows observable and/or measurable reduction in or absence of one or more signs and symptoms of the particular disease. The present technology also provides for methods of administering one or more compounds of the present technology to a patient in an effective amount for the treatment or prophylaxis of a disease such as, for example, an alphavirus infection.

The subject compounds are structurally related and take the form of Formula I and Formula II:

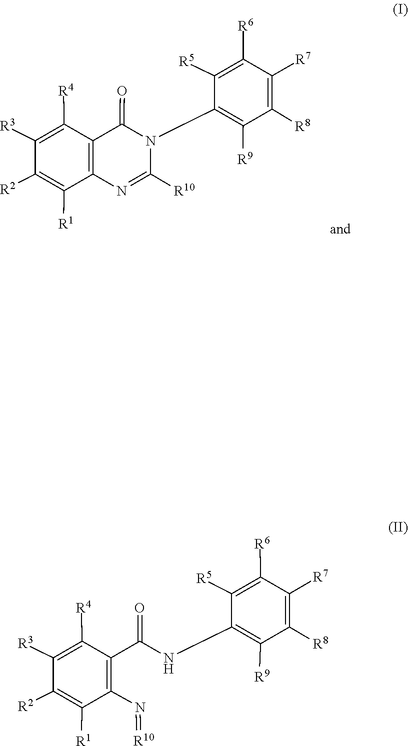

wherein:

R¹, R², R³ and R⁴ are the same or different and are independently selected from hydrogen, halogen, alkyl, C₃-C₈-cycloalkyl, halogen-substituted alkyl, alkoxy, halogen-substituted alkoxy, nitro, cyano, aryl, alkyl-substituted aryl, alkoxy-substituted aryl, halogen-substituted aryl, saturated or unsaturated 4- to 6-membered heterocyclyl, sulfonyl, and sulfonamidyl,

provided that not all of R¹, R², R³ and R⁴ are simultaneously hydrogen, and

provided that at least one of R¹, R², R³ and R⁴ is an electron-withdrawing group;

R⁵, R⁶, R⁷, R⁸, and R⁹ are the same or different and are independently selected from hydrogen, halogen, alkyl, unsubstituted or substituted C₃-C₈-cycloalkyl, halogen-substituted alkyl, alkoxy, halogen-substituted alkoxy, azido, nitro, and cyano;

R¹⁰ is selected from aliphatic amino, aliphatic alkylamino,

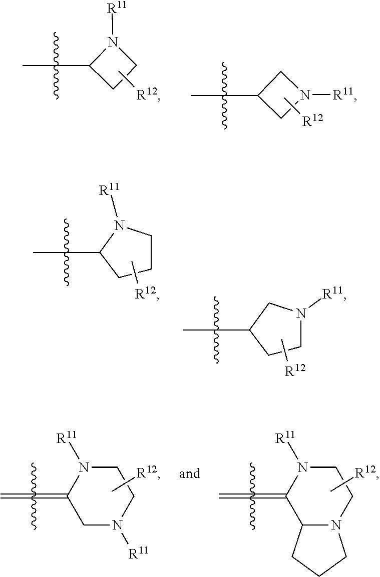

wherein:

R¹¹ is selected from hydrogen, halogen, alkyl, halogen-substituted alkyl, alkoxy, halogen-substituted alkoxy, unsubstituted or substituted C₃-C₈-cyclic ether, and unsubstituted or C-substituted or N-substituted saturated or unsaturated nitrogen C₄-C₈ heterocyclyl;

R¹² is selected from hydrogen, halogen, alkyl, halogen-substituted alkyl, alkoxy, and halogen-substituted alkoxy;

and salts thereof.

The salts may be any salts or pharmaceutically suitable salts as described herein.

Note that Formula (II) is essentially a ring-opened form of Formula I.

For the Formula I compounds, it is generally preferred that R¹⁰ is selected from

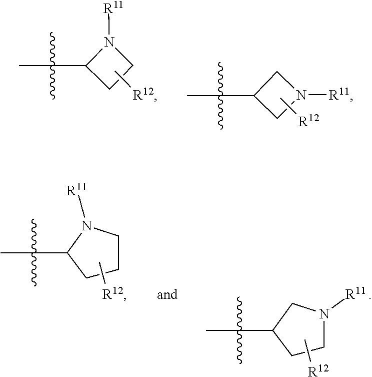

For the Formula II compounds, it is generally preferred that R¹⁰ is selected from

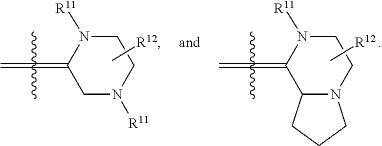

As noted above, the disclosure also includes a pharmaceutical composition for inhibiting a viral infection, the composition comprising an effective amount of one or more compounds disclosed herein. Also disclosed, is a corresponding method of inhibiting a viral infection comprising administering to a subject an antiviral-effective amount of a compound as recited herein.

While not wishing to be bound by theory, it is believed that the compounds and compositions disclosed herein act through a post-entry, viral-specific, mechanism of action by inhibiting viral replication through direct interaction with the nsP2 and nsP4 and inhibiting their activities during replication of the genome, resulting in the prevention or treatment of diseases related to an encephalitic alphavirus. “Virus specific” means the compounds do not use host cellular machinery to inhibit virus. Thus, there are fewer off-target effects because the compounds target the virus and not host.

In another aspect, a composition is provided including a compound of Formulas I or II, optionally in combination with a pharmaceutically acceptable carrier. In a related aspect, a pharmaceutical composition is provided for treating a viral infection, where the composition includes an effective amount of the compound of Formulas I or II. In some embodiments, the viral infection comprises an alphavirus. In some embodiments, the viral infection comprises an encephalitic alphavirus. In some embodiments, the viral infection comprises a Venezuelan equine encephalitis virus. In some embodiments, the effective compound selectively treats the viral infection. Thus, the instant present technology provides pharmaceutical compositions and medicaments comprising any of the compounds disclosed herein and a pharmaceutically acceptable carrier or one or more excipients or fillers. The compositions may be used in the methods and treatments described herein. Such compositions include a viral inhibitory effective amount of any compound as described herein, including but not limited to a compound of Formulas I or II. In some embodiments, the pharmaceutical composition is packaged in unit dosage form. The unit dosage form is effective in preventing infection by, reducing symptoms associated with, and/or reducing risk of transmission of an encephalitic alphavirus when administered to a subject in need thereof.

The pharmaceutical compositions may be prepared by mixing one or more compounds of the present technology, pharmaceutically acceptable salts thereof, stereoisomers thereof, tautomers thereof, or solvates thereof, with pharmaceutically acceptable carriers, excipients, binders, diluents or the like to prevent and treat disorders associated with the effects of increased plasma and/or hepatic lipid levels. The compounds and compositions described herein may be used to prepare formulations and medicaments that prevent or treat a variety of disorders associated with an encephalitic alphavirus. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of compounds of the present technology by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering a compound of the present technology to a patient in increasing amounts until the elevated plasma or elevated white blood cell count or hepatic cholesterol or triglycerides or progression of the disease state is decreased or stopped. The progression of the disease state can be assessed using in vivo imaging, as described, or by taking a tissue sample from a patient and observing the target of interest therein. The compounds of the present technology can be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the condition being treated and the pharmacological activity of the compound being used. The determination of optimum dosages for a particular patient is well known to those skilled in the art.

Various assays and model systems can be readily employed to determine the therapeutic effectiveness of the viral treatment according to the present technology.

Effectiveness of the compositions and methods of the present technology may also be demonstrated by a decrease in the symptoms of an encephalitic alphavirus, such as, for example, fever, headache, and encephalitis.

For each of the indicated conditions described herein, test subjects will exhibit a 10%, 20%, 30%, 50% or greater reduction, up to a 75-90%, or 95% or greater, reduction, in one or more symptom(s) caused by, or associated with, viral infection in the subject, compared to placebo-treated or other suitable control subjects.

The compounds of the present technology can also be administered to a patient along with other conventional therapeutic agents that may be useful in the treatment or prophylaxis of viral infection. In one aspect, a method is provided for administering to a subject in need thereof an antiviral effective amount of a compound of the present technology. A subject in need thereof may be a patient suffering from or believed to be at risk of suffering from a disease associated with a virus, such as an encephalitic alphavirus. In some embodiments, the compound is effective in the treatment of an alphavirus. In some embodiments, the compound is effective in the treatment of an encephalitic alphavirus. In some embodiments, the compound is effective in the treatment of a Venezuelan equine encephalitis virus. In some embodiments, the compound selectively treats the viral infection. In some embodiments, the risk of infection by and/or transmission of an alphavirus by said subject is decreased. In any of these embodiments, the administration may include oral administration, parenteral administration, or nasal administration. In any of these embodiments, the administration may include subcutaneous injections, intravenous injections, intraperitoneal injections, or intramuscular injections. In any of these embodiments, the administration may include oral administration. The methods of the present technology can also comprise administering, either sequentially or in combination with one or more compounds of the present technology, a conventional therapeutic agent in an amount that can potentially or synergistically be effective for the treatment or prophylaxis of encephalitic alphavirus. Exemplary therapeutic agents for use in combination therapies with one or more compounds of the present technology include, but are not limited to, other antiviral therapeutics, antibiotics, and anti-inflammatory drugs.

In one aspect, a compound of the present technology is administered to a patient in an amount or dosage suitable for therapeutic use. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

A compound of the present technology can also be modified, for example, by the covalent attachment of an organic moiety or conjugate to improve pharmacokinetic properties, toxicity or bioavailability (e.g., increased in vivo half-life). The conjugate can be a linear or branched hydrophilic polymeric group, fatty acid group or fatty acid ester group. A polymeric group can comprise a molecular weight that can be adjusted by one of ordinary skill in the art to improve, for example, pharmacokinetic properties, toxicity or bioavailability. Exemplary conjugates can include a polyalkane glycol (e.g., polyethylene glycol (PEG), polypropylene glycol (PPG)), carbohydrate polymer, amino acid polymer or polyvinyl pyrolidone and a fatty acid or fatty acid ester group, each of which can independently comprise from about eight to about seventy carbon atoms. Conjugates for use with a compound of the present technology can also serve as linkers to, for example, any suitable substituents or groups, radiolabels (marker or tags), halogens, proteins, enzymes, polypeptides, other therapeutic agents (for example, a pharmaceutical or drug), nucleosides, dyes, oligonucleotides, lipids, phospholipids and/or liposomes. In one aspect, conjugates can include polyethylene amine (PEI), polyglycine, hybrids of PEI and polyglycine, polyethylene glycol (PEG) or methoxypolyethylene glycol (mPEG). A conjugate can also link a compound of the present technology to, for example, a label (fluorescent or luminescent) or marker (radionuclide, radioisotope and/or isotope) to comprise a probe of the present technology. Conjugates for use with a compound of the present technology can, in one aspect, improve in vivo half-life. Other exemplary conjugates for use with a compound of the present technology as well as applications thereof and related techniques include those generally described by U.S. Pat. No. 5,672,662, which is hereby incorporated by reference herein.

In another aspect, the present technology provides methods of identifying a target of interest including contacting the target of interest with a detectable or imaging effective quantity of a labeled compound of the present technology. A detectable or imaging effective quantity is a quantity of a labeled compound of the present technology necessary to be detected by the detection method chosen. For example, a detectable quantity can be an administered amount sufficient to enable detection of binding of the labeled compound to a target of interest including, but not limited to, VEEV nsP2 (not a helicase). Suitable labels are known by those skilled in the art and can include, for example, radioisotopes, radionuclides, isotopes, fluorescent groups, biotin (in conjunction with streptavidin complexation), and chemoluminescent groups. Upon binding of the labeled compound to the target of interest, the target may be isolated, purified and further characterized such as by determining the amino acid sequence.

The terms “associated” and/or “binding” can mean a chemical or physical interaction, for example, between a compound of the present technology and a target of interest. Examples of associations or interactions include covalent bonds, ionic bonds, hydrophilic-hydrophilic interactions, hydrophobic-hydrophobic interactions and complexes. Associated can also refer generally to “binding” or “affinity” as each can be used to describe various chemical or physical interactions. Measuring binding or affinity is also routine to those skilled in the art. For example, compounds of the present technology can bind to or interact with a target of interest or precursors, portions, fragments and peptides thereof and/or their deposits.

The examples herein are provided to illustrate advantages of the present technology and to further assist a person of ordinary skill in the art with preparing or using the compounds of the present technology or salts, pharmaceutical compositions, derivatives, metabolites, prodrugs, racemic mixtures or tautomeric forms thereof. The examples herein are also presented in order to more fully illustrate the preferred aspects of the present technology. The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

### EXAMPLES

### General Synthetic and Analytical Details

¹H and ¹³C NMR spectra were recorded on a Bruker AM 400 spectrometer (operating at 400 and 101 MHz respectively) or a Bruker AVIII spectrometer (operating at 500 and 126 MHz respectively) in CDCl₃ with 0.03% TMS as an internal standard or DMSO-d₆. The chemical shifts (6) reported are given in parts per million (ppm) and the coupling constants (J) are in Hertz (Hz). The spin multiplicities are reported as s=singlet, bs=broad singlet, d=doublet, t=triplet, q=quartet, dd=doublet of doublet and m=multiplet. The LCMS analysis was performed on an Agilent 1200 RRL chromatograph with photodiode array UV detection and an Agilent 6224 TOF mass spectrometer. The chromatographic method utilized the following parameters: a Waters Acquity BEH C-18 2.1×50 mm, 1.7 um column; UV detection wavelength=214 nm; flow rate=0.4 ml/min; gradient=5-100% acetonitrile over 3 minutes with a hold of 0.8 minutes at 100% acetonitrile; the aqueous mobile phase contained 0.15% ammonium hydroxide (v/v). The mass spectrometer utilized the following parameters: an Agilent multimode source which simultaneously acquires ESI+/APCI+; a reference mass solution consisting of purine and hexakis(1H, 1H, 3H-tetrafluoropropoxy)phosphazine; and a make-up solvent of 90:10:0.1 MeOH:Water:Formic Acid which was introduced to the LC flow prior to the source to assist ionization. Melting points were determined on a Stanford Research Systems OptiMelt apparatus.

### Assay Conditions

### HTS Primary and Confirmatory Assay Using TC-83.

Cell Culture: Vero 76 cells obtained from ATCC (CRL-1587) were cultured and maintained in MEM-E (Invitrogen, 10370-088) with 10% Hi-FBS (Invitrogen 16000), 1% Penicillin/Streptomycin/L-glutamine (Invitrogen 10378-024) and 1% HEPES (Invitrogen 15630-080). The cells are maintained at 37° C., 5.0% CO₂ to 100% confluence being passaged 1:4 every 3-4 days. For cell plating, cells were detached from flask bottom by using Trypsin-EDTA solution and then re-suspended in a growth media. Cells were passaged no more than ten times after being thawed.

VEEV culture: VEEV TC-83 was used for screening. The VEEV stock was prepared in Vero76 cells using an initial stock obtained from Dr. Chung.

Compound Dosing/Plating: The positive control was MPA at 10 uM final well concentration. The compounds were diluted in complete growth medium to 6× concentrated dosing solution which was dispensed into 384-well black clear-bottom tissue culture treated plates (5 μL volume).

Single Dose Compound Preparation: The MLSMR library was plated at 25 μM single dose concentration.

Dose Response Compound Preparation: The compounds were tested in a dose response format using a 1:2 serial dilution with the highest concentrations starting at 25 μM and extending to 0.05 μM over a 10-plate 1:2 serial dilution pattern. DMSO and compounds were diluted in assay media to 6× and 5 μL was dispensed to assay plates. The final DMSO in the assay for all screening concentrations was 0.25%.

Virus Addition: VEEV stock was diluted in the culture media to 6.44 pfu/ml. (MOI 0.05 μL).

VEEV and Cell Plating: 3,000 cells/well alone or with VEEV virus at the previously indicated dilution (180,000 cells/ml) were plated in 25 uL using a Matrix WellMate. All additions were done using a Matrix WellMate housed in a class II Biosafety Cabinet within the BSL-2 laboratory. The plates were incubated in an actively humidified incubator with 5.0% CO₂ at 37° C. for 72 h and 95% humidity.

Endpoint Read: The assay plates were equilibrated to room temperature for 30 minutes and then an equal volume of CellTiter-Glo reagent (Promega Inc.) was added to each well. Plates were incubated for 10 min at room temperature and luminescence was measured using a Perkin Elmer Envision multi-label reader.

Cell-Based Confirmatory Screen for Compounds that Inhibit VEEV, TC-83.

Cell Culture: Vero 76 (CRL-1587, ATCC) were purchased from ATCC and maintained in 37° C. incubator with 5% CO₂. The cells were cultured in a complete media (Minimum Essential Media with Earle's salt and 10% fetal bovine serum). Cells were passaged once a week and harvested from flasks using 0.05% trypsin-EDTA.

Assay Media—Preparation of Complete DMEM media: 5 mL Pen/Strep (Gibco, Cat. No. 15149) and 50 mL of heat-inactivated FBS (Gibco, Cat. No. 10082147) was added to 500 mL of Dulbecco's Modified Eagle Medium (Gibco, Cat. No. 11995-073). Virus: TC-83 strain was obtained from Dr. Brett Beitzel from United State Army Research Medical Research Institute for Infectious diseases and amplified in BHK C-21 cell line once.

Dose Response Compound Preparation: For dose response screening, compounds or carrier control (DMSO) were diluted to 3× in Complete DMEM media. Test compounds were serially diluted 1:2 resulting in an 8 point dose response dilution series. (final plate well concentration ranging from 50 uM to 0.39 μM and a final DMSO concentration of 0.25%). 30 μl of each dilution was dispensed to assay plates (0.75% DMSO) in duplicate.

Control Drug: The positive control drug for this assay, mycophenolic acid was solubilized in DMSO. It was diluted and added to the assay plates as described for test compounds. Final concentration for ribavirin was 10 μM. All wells contained 0.25% DMSO.

Assay Set up: Vero 76 cells were plated in 96-well plates at a density of 15,000 cells per well in a volume of 45 μL of DMEM complete media. The cells were grown for 24 hours prior to testing in a 5% CO₂, 37° C. cell culture incubator. Viruses, TC-83 strain, was diluted in cell culture medium to be 750 pfu/15 uL (0.05 MOI) and then added to the plates at a volume of 15 μL per well. The plates were incubated for 48 hours in a 37° C. incubator with 5% CO₂.

Endpoint Read: Following the two day incubation period, the assay plates were equilibrated to room temperature for 10 min and an equal volume (90 μL) of Cell Titer-Glo reagent (Promega Inc.) was added to each well using a Microflow (Biotek, VT) and plates were incubated for an additional 10 min at room temperature. At the end of the incubation, luminescence was measured using a Synergy4 Multimode plate reader (Biotek, VT) with an integration time of 0.2 s.

Data Analysis: Results are reported as percent (%) CPE inhibition and were calculated using the following formula: % CPE inhibition=100*(Test Cmpd-Med Virus)/(Med Cells-Med Virus). Four ribavirin positive control wells were included on each plate for quality control purposes. To quantify the viral cytopathic effect, IC50s were calculated for each substance using the 4 parameter Levenburg-Marquardt algorithm with the minimum and maximum parameters locked at 0 and 100, respectively.

Cell-Based Confirmatory Screen for Compounds that Inhibit VEEV, V3526.

Cell Culture: Vero 76 (CRL-1587, ATCC) were purchased from ATCC and maintained in 37° C. incubator with 5% CO₂. The cells were cultured in a complete media (Minimum Essential Media with Earle's salt and 10% fetal bovine serum). Cells were passaged once a week and harvested from flasks using 0.05% trypsin-EDTA. Assay Media—Preparation of Complete DMEM media: 5 mL Pen/Strep (Gibco, Cat. No. 15149) and 50 mL of heat-inactivated FBS (Gibco, Cat. No. 10082147) was added to 500 mL of Dulbecco's Modified Eagle Medium (Gibco, Cat. No. 11995-073).

Virus: V3526 strain. Virus was rescued from BHK C-21 cells that were transfected with infectious V3526 RNA. The recued virus was amplified in BHK C-21 cells once and then used as a stock virus.

Dose Response Compound Preparation: For dose response screening, compounds or carrier control (DMSO) were diluted to 3× in Complete DMEM media. Test compounds were serially diluted 1:2 resulting in an 8 point dose response dilution series. (final plate well concentration ranging from 50 μM to 0.39 μM and a final DMSO concentration of 0.25%). 30 μL of each dilution was dispensed to assay plates (0.75% DMSO) in duplicate.

Control Drug: The positive control drug for this assay, mycophenolic acid was solubilized in DMSO. It was diluted and added to the assay plates as described for test compounds. Final concentration for ribavirin was 10 μM. All wells contained 0.25% DMSO.

Assay Set up: Vero 76 cells were plated in 96-well plates at a density of 15,000 cells per well in a volume of 45 μL of DMEM complete media. The cells were grown for 24 hours prior to testing in a 5% CO₂, 37° C. cell culture incubator. V3526 VEEV virus, was diluted in cell culture medium to be 750 pfu/15 uL (0.05 MOI) and then added to the plates at a volume of 15 μL per well. The plates were incubated for 48 hours in a 37° C. incubator with 5% CO₂.

Endpoint Read: Following the two day incubation period, the assay plates were equilibrated to room temperature for 10 min and an equal volume (90 μL) of Cell Titer-Glo reagent (Promega Inc.) was added to each well using a Microflow (Biotek, VT) and plates were incubated for an additional 10 min at room temperature. At the end of the incubation, luminescence was measured using a Synergy4 Multimode plate reader (Biotek, VT) with an integration time of 0.2 s.

Cell-Based Secondary Assay for Compounds that Inhibit VEEV, TC-83 and Other Alphaviruses (Trinidad Donkey).

Biosafety and Biosecurity: All experiments with VEEV Trinidad donkey (TrD) strain was done in the Regional Biocontainment Laboratory (RBL) in University of Louisville. All procedures were done in compliance with Select Agent Rules. Cell Culture: Vero 76 cells obtained from ATCC (CRL-1586) were cultured and maintained in Dulbecco's Modified Eagle's Medium (DMEM) with 4500 mg/L glucose, 2 mM L-glutamine, and 10% FBS (culture media). The cells are maintained at 37° C., 5.0% CO₂ to 100% confluence being passaged every three to seven days. For cell plating, cells were detached from flask bottom by using 0.05% Trypsin-EDTA solution and then re-suspended in a growth media.

VEEV culture: VEEV TrD strain was used for screening. The VEEV TrD stock was prepared in Vero 76 cells using an initial stock obtained from World Reference Center for Emerging Viruses and Arboviruses (Dr. Robert Tesh). Briefly, cells were grown in two T-175 flasks to 50% confluence in a culture media. The cells were infected with 1 mL of diluted virus stock (1:10 dilution of the original stock) per T175 for 1.5 hours and then washed, and replenished with 25 mL media. The cells were incubated for 2 days in an incubator at 37° C., 5% CO₂ and high humidity. The supernatant was harvested and the cell debris pelleted by centrifuging at 1,000 rpm for 5 minutes at 18° C. The supernatant was aliquoted (1 mL per tube) and stored at −80° C. These virus stocks were titrated in Vero 76 cells using an agarose overlay plaque method and the titers were 2.0×E10 pfu/mL.

Dose Response Compound Preparation: The compounds were tested in a dose response format using a 1:2 serial dilution with the highest concentrations starting at 12.5 μM and extending to 0.09 μM over 8 points 1:2 serial dilution pattern. DMSO and compounds were diluted in assay media to 3× and 30 μL was dispensed to assay plates (see below). The final DMSO in the assay for all screening concentrations was 0.25%. Assay Set up: 45 μl of Vero 76 cell suspension (267,000 cells/mL) were plated in clear bottom black well 96-well plates and the plates were incubated in an incubator at 37° C. in a humidified 5% CO₂ atmosphere. The next day, thirty uL of drugging media (0.75% DMSO) were added to the each wells and the plates were incubated at 37° C. for an hour. The plates were transferred into a BSL-3 lab in the RBL. Each well received fifteen L of VEEV TrD virus diluted in Complete media (40,000 pfu/mL, final 0.05 MOI). For the cell control wells, Complete media were added instead of virus solution. Drug plating was conducted using a EVO100, 96 MAC (Tecan) and virus solution was added using MicroFlo (Biotek). The assay plates were incubated for two days at 37° C., 5% CO₂ and 90% relative humidity.

Endpoint Read: The assay plates were equilibrated to room temperature for 30 minutes and then an equal volume of CellTiter-Glo reagent (Promega Inc.) was added to each well. Plates were incubated for 10 min at room temperature and luminescence was measured using a Synergy4 HT multi-label reader.

### Vero76 Cytotoxicity Assay for VEEV Compounds

Cell Culture: Vero 76 cells obtained from ATCC (CRL-1587) were cultured and maintained in MEM-E (Invitrogen, 10370-088) with 10% Hi-FBS (Invitrogen 16000), 1% Penicillin/Streptomycin/L-glutamine (Invitrogen 10378-024) and 1% HEPES (Invitrogen 15630-080). The cells are maintained at 37° C., 5.0% CO₂ to 100% confluence being passaged 1:4 every 3-4 days. For cell plating, cells were detached from flask bottom by using Trypsin-EDTA solution and then re-suspended in a growth media. Cells were passaged no more than ten times after being thawed.

Compound Dosing/Plating: Carrier control/compounds were diluted in complete growth medium to prepare a 6× concentrated dosing solution which was dispensed into 384-well black clear-bottom tissue culture treated plates (5 μL volume).

Cell Plating: Twenty-five uL of complete growth medium containing 3000 cells were dispensed per well. Plates were incubated at 37° C., 5% CO₂ for 72 h prior to endpoint detection.

Endpoint/Detection: At the end of the treatment period, assay plates were removed from the incubator and equilibrated to room temperature for 10 min. Thirty uL of Cell Titer Glo reagent was added and plates were incubated for an additional 10 min in the dark. At the end of the incubation, assay plates were analyzed using a PerkinElmer Envision microplate reader in luminescence mode with an integration time of 0.1 s.

Virus Titer Reduction Secondary Screen for Compounds that Inhibit VEEV (Strain Trinidad Donkey)

Biosafety and Biosecurity: All experiments with VEEV Trinidad donkey strain was done in the Regional Biocontainment Laboratory in University of Louisville. All procedures were done in compliance with Select Agent Rules.

Cell Culture: Vero 76 cells (ATCC; CRL1586) were cultured in Dulbecco's Modified Eagle's Medium (DMEM) with 4500 mg/L glucose, 2 mM L-glutamine, and 10% FBS (culture media). The cells are maintained at 37° C., 5.0% CO₂ to 100% confluence being passaged every three to seven days. For cell plating, cells were detached from flask bottom by using 0.05% Trypsin-EDTA solution and then re-suspended in a growth media.

VEEV culture: Trinidad Donkey (TrD) was used for screening. The VEEV TrD stock was prepared in Vero 76 cells using an initial stock obtained from World Reference Center for Emerging Viruses and Arboviruses (Dr. Robert Tesh). Briefly, cells were grown in two T-175 flasks to 50% confluence in a culture media. The cells were infected with 1 mL of diluted virus stock (1:10 dilution of the original stock) per T175 for 1.5 hours and then washed, and replenished with 25 mL media. The cells were incubated for 2 days in an incubator at 37° C., 5% CO₂ and high humidity. The supernatant was harvested and the cell debris pelleted by centrifuging at 1,000 rpm for 5 minutes at 18° C.

The supernatant was aliquoted (1 ml per tube) and stored at −80° C. These virus stocks were titrated in Vero 76 cells using an agarose overlay plaque method and the titer was 2.0×E10 pfu/ml.

Cell Plating: Vero 76 cells were seeded at 70% confluence in a 12-well plate in a volume of 1 mL and incubated for overnight at 37° C. with 5% CO₂ and high humidity. Virus Addition: The cells were infected with virus by adsorption for an hour. Cell culture media in the 12-well plates were removed completely and the cells were infected with either mock virus (media only) or VEEV. VEEV stock was diluted in the culture media to 9×E03 pfu/ml and 200 μL was added to the test wells and the virus control wells (final MOI of 0.1). The plates were incubated in an actively humidified incubator with 5.0% CO₂ at 37° C. for one hour. During the incubation, plates were gently rocked every 20 min. to ensure the coverage of the cells with virus. After the adsorption, the cells were rinsed 1 mL of PBS per well and then replenished with the media containing testing articles. All additions were performed in a class II Biosafety Cabinet. The plates were incubated in an actively humidified incubator with 5.0% CO₂ at 37° C. for 48 h.

Control and Drug Preparation: Carrier Control consisted of DMSO diluted in assay media to 0.25% and 1000 μL was dispensed to both cell and virus control wells of 12-well tissue culture treated plates. Test compounds were diluted in media to be at target concentration with a DMSO concentration of 0.25%.

Titration of Progeny viruses (Mini plaque assay): Titer of progeny viruses produced from the cell was measured by a mini plaque assay in 96-well plate format. Fresh Vero 76 cells were seeded and grown in 96-well plates overnight. The cell culture supernatants from the 96-well plates were emptied and the cells were infected with 25 μL of 10-fold serial dilutions of progeny virus containing medium from respective samples (drug treated or untreated). The plates were incubated for one hour in an incubator at 37° C., 5% CO₂. The cells were rinsed with 100 μL per well of PBS once and then replenished with DMEM with 0.75% methyl-cellulose and 10% FBS. The cell plates were incubated at 37° C., 5% CO₂, and high humidity for an additional three days. Crystal violet solution with 4% paraformaldehyde was used to developed plaques in the wells. The assay plates were equilibrated to room temperature for 10 minutes and then an equal volume of the crystal violet solution was added to each well. The plates were incubated for 60 min at room temperature and stained one more time. After a wash the plates with water, the number of plaques in each wells were determined by a visual counting. Virus titers were calculated by: No. of plaques×10E (dilution fold at the counting)*1000/25 (pfu/mL). Compounds treatment were done in a duplicate independently and mean from duplicates of titration was used. Log reduction of titer was calculated by: Log ₁₀ (titer of Pos control)−Log ₁₀ (titer of sample).

### Counter Screen of Venezuelan Equine Encephalitis Virus (VEEV) Inhibitors in a Cell-Based Anti-Respiratory Syncitial Virus (RSV) Assay

Cell Culture: HEp-2 cells (ATCC CCL-23, American Tissue Culture Type) were maintained as adherent cell lines in DMEM with 2 mM L-glutamine and 10% fetal bovine serum (FBS) at 37° C. in a humidified 5% CO₂ atmosphere. Cells were passaged as needed and harvested from flasks using 0.05% trypsin-EDTA.

Assay Media—Preparation of Complete DMEM/F12: DMEM/F12 (Invitrogen, Cat. No. 11320) was supplemented with 5 mL of Pen/Strep (Invitrogen, Cat. No. 10378016), 5 mL of 200 mM glutamine (Invitrogen, Cat No. 25030-081), and 10 mL of HI-FBS was added per 500 mL of media.

RSV culture: Human respiratory syncytial virus (HRSV) strain Long (ATCC VR-26) was used for screening. The RSV stock was prepared in HEp-2 cells using an initial stock obtained from ATCC. Briefly, HEp-2 cells were grown in two T-175 flasks to 50% confluence in Dulbecco's Modified Eagle Medium: Nutrient Mixture F-12 (DMEM/F12), pH 7.5 with 2.5 mM L-glutamine, 2% FBS and 125 U of penicillin, 125 ug of streptomycin per ml. 0.2 mL of RSV was added to 25 ml of CDMEM/F12. After three days incubation at 37° C., 5% CO₂ and high humidity, the supernatant was harvested and the cell debris pelleted by centrifuging at 1,000 rpm for 5 minutes at 18° C. Trehalose and FBS were added to a final concentration of 10% each and the supernatant was aliquoted (1 ml per tube) and stored at −80° C. These virus stocks were titrated in HEp-2 cells using an agarose overlay plaque method and the titer was 1.0 E7 pfu/ml.

Dose Response Compound Preparation: For dose response screening, compounds or carrier control (DMSO) were diluted to 3× in Complete DMEM/F12. Test compounds were serially diluted 1:2 resulting in an 8 point dose response dilution series. (final plate well concentration ranging from 50 μM to 0.39 μM and a final DMSO concentration of 0.25%). 39 μL of each dilution was dispensed to assay plates (0.75% DMSO) in triplicate.

Control Drug: The positive control drug for this assay, MPA was solubilized in DMSO.

Preparation of HEp-2 Cells: Cells were Harvested and Resuspended to 267,000 Cells Per Ml in Complete DMEM/F12.

Assay Set up: Forty five ul of HEp-2 cell suspension (12,000 cells/well) were plated in clear bottom black well 96-well plates and the plates were incubated in an incubator at 37° C. in a humidified 5% CO₂ atmosphere. The next day, 30 μL of drugging media (0.75% DMSO) were added to the each wells and the plates were incubated at 37° C. for an hour. Each well received 15 μL of RSV diluted in Complete DMEM/F12 media (40,000 pfu/mL, final 0.05 MOI). For the cell control wells, Complete DMEM/F12 media were added instead of virus solution. Drug plating was conducted using a EVO100, 96 MAC (Tecan) and virus was added using MicroFlo (Biotek). The assay plates were incubated for five days at 37° C., 5% CO₂ and 90% relative humidity.

Endpoint Read: The assay plates were equilibrated to room temperature for 30 minutes and then an equal volume of CellTiter-Glo reagent (Promega Inc.) was added to each well. Plates were incubated for 10 min at room temperature and luminescence was measured using a Synergy4 multi-label reader.

### HEp2 Cytotoxicity Assay for VEEV Compounds

Cell Culture: HEp-2 cells (ATCC CCL-23, American Tissue Culture Type) were maintained as adherent cell lines in DMEM with 2 mM L-glutamine and 10% fetal bovine serum (FBS) at 37° C. in a humidified 5% CO₂ atmosphere. Cells were passaged as needed and harvested from flasks using 0.05% trypsin-EDTA.

Assay Media—Preparation of Complete DMEM/F12: DMEM/F12 (Invitrogen, Cat. No. 11320) was supplemented with 5 mL of Pen/Strep (Invitrogen, Cat. No. 10378016), 5 mL of 200 mM glutamine (Invitrogen, Cat No. 25030-081), and 10 mL of HI-FBS was added per 500 mL of media.

Dose Response Compound Preparation: For dose response screening, compounds or carrier control (DMSO) were diluted to 3× in Complete DMEM/F12. Test compounds were serially diluted 1:2 resulting in an 8 point dose response dilution series. (final plate well concentration ranging from 50 μM to 0.39 μM and a final DMSO concentration of 0.25%). 30 μL of each dilution was dispensed to assay plates (0.75% DMSO) in triplicate.

Control Drug: The positive control drug for this assay, MPA was solubilized in DMSO.

Preparation of HEp-2 cells: Cells were harvested and resuspended to 267,000 cells per ml in Complete DMEM/F12.

Assay Set up: Forty five ul of HEp-2 cell suspension (12,000 cells/well) were plated in clear bottom black well 96-well plates and the plates were incubated in an incubator at 37° C. in a humidified 5% CO₂ atmosphere. The next day, 30 μL of drugging media (0.75% DMSO) were added to the each wells and the plates were incubated at 37° C. for an hour. Each well then received 15 μL of Complete DMEM/F12 media. For the cell control wells, Complete DMEM/F12 media were added instead of virus solution. Drug plating was conducted using a EVO100, 96 MAC (Tecan) and virus was added using MicroFlo (Biotek). The assay plates were incubated for five days at 37° C., 5% CO₂ and 90% relative humidity.

Endpoint Read: The assay plates were equilibrated to room temperature for 30 minutes and then an equal volume of CellTiter-Glo reagent (Promega Inc.) was added to each well. Plates were incubated for 10 min at room temperature and luminescence was measured using a Synergy4 multi-label reader.

Cell-Based Secondary Assay for Compounds that Inhibit VEEV, TC-83 and Other Alphaviruses (Chikungunya Virus).

Cell Culture: Vero 76 cells obtained from ATCC (CRL-1587) were cultured and maintained in MEM-E (Invitrogen, 10370-088) with 10% Hi-FBS (Invitrogen 16000), 1% Penicillin/Streptomycin/L-glutamine (Invitrogen 10378-024) and 1% HEPES (Invitrogen 15630-080). The cells are maintained at 37° C., 5.0% CO₂ to 100% confluence being passaged 1:4 every 3-4 days. For cell plating, cells were detached from flask bottom by using Trypsin-EDTA solution and then re-suspended in a growth media. Cells were passaged no more than ten times after being thawed.

Compound Dosing/Plating: No positive control. The compounds were diluted in complete growth medium to 6× concentrated dosing solution which was dispensed into 96-well black clear-bottom tissue culture treated plates (25 μL volume).

Dose Response Compound Preparation: The compounds were tested in a dose response format using a 1:2 serial dilution with the highest concentrations starting at 100 M and extending to 0.78 μM over a 8-dose 1:2 serial dilution pattern. DMSO and compounds were diluted in assay media to 4× and 25 μL was dispensed to assay plates. The final DMSO in the assay for all screening concentrations was 0.25%. Virus Addition: CHIKV stock was diluted in the culture media to 100 TCID50s/25 μL, and 25 μL were added to each test well.

Cell Plating and virus addition: 6,000 cells/well (120,000 cells/ml) were plated in 50 uL using a Matrix WellMate. All additions were done using a Matrix WellMate housed in a class II Biosafety Cabinet within the BSL-2 laboratory. The plates were incubated overnight in an actively humidified incubator with 5.0% CO₂ at 37° C. for 18 h and 95% humidity. Compounds (25 μL) were added after the cells had adhered to the plate, and CHIKV virus was added immediately after compound addition. The plates were incubated for 72 h in an actively humidified incubator with 5.0% CO₂ at 37° C. for 72 h and 95% humidity, and then endpoint reagent was added.

Endpoint Read: The assay plates were equilibrated to room temperature for 30 minutes and then 100 μL of CellTiter-Glo reagent (Promega Inc.) was added to each well. Plates were incubated for 10 min at room temperature and luminescence was measured using a Perkin Elmer Envision multi-label reader.

General Chemical Methods. Reagents or intermediates not described below were purchased from commercial vendors. Purity of all final compounds was confirmed by HPLC/MS analysis and determined to be ≥95%.

¹H and ¹³C NMR spectra were recorded on a Varian Unity-Inova 400 MHz NMR Spectrometer (operating at 400 and 101 MHz, respectively) or a Varian Unity-Inova 500 MHz NMR Spectrometer (operating at 500 and 126 MHz, respectively) or Bruker Ascend 400 MHz Spectrometer (operating at 400 and 101 MHz, respectively) in CDCl₃ (residual internal standard CHCl₃=δ 7.26), DMSO-d₆ (residual internal standard CD₃SOCD₂H=δ 2.50), or acetone-d₆ (residual internal standard CD₃COCD₂H=δ 2.05).

The chemical shifts (6) reported are given in parts per million (ppm), and the coupling constants (J) are in hertz (Hz). The spin multiplicities are reported as s=singlet, brs=broad singlet, d=doublet, t=triplet, q=quartet, p=pentuplet, dd=doublet of doublet, dt=doublet of triplet, td=triplet of doublet, tt=triplet of triplet, and m=multiplet.

The LC-MS analysis was performed on an Agilent 1290 Infinity II HPLC system with 1290 Infinity II Diode Array Detector and an Agilent 6120 Quadrupole LC-MS system. The analytical chromatography method utilized the following parameters: Poroshell 120 EC-C18, 1.9 μm column, UV detection wavelength=254 nm, Flow rate=1.0 mL/min, Gradient=5-100% LC-MS grade methanol over 4 min; The organic mobile phase and aqueous mobile phase contained 0.1% LC-MS grade formic acid. The mass spectrometer utilized the following parameters: an Agilent multimode source that simultaneously acquires ESI+/APCI+; Final compounds were determined to be >95% purity by UV-LCMS at 254 nm. The purity analysis was performed on Waters Prep LC-150 HPLC system with Waters 2545 Binary Gradient Module Pump, and Waters 2998 Photodiode Array Detector. The analytical chromatography method utilized the following parameters: XBridge® C18, 4.6×50 mm, 3.5 m column; UV detection wavelength=254 nm, Flow rate=1.0 mL/min, Gradient=5-100% HPLC grade Methanol over 10 min; The preparative chromatography method utilized the following parameters: XBridge® Prep C18, 19×150 mm, 5.0 m column; UV detection wavelength=254 nm, Flow rate=15 mL/min, Gradient=5-100% HPLC grade Methanol over 45 min; The organic mobile phase and aqueous mobile phase contained 0.1% HPLC grade ammonium chloride. Compound purity was also determined by using an Agilent 1100 HPLC system with photodiode array UV detection. The chromatographic method utilized either of two methods: (option 1) an OD-H chiral column, 19 mm×150 mm, 5 μm column, UV detection wavelength=254 nm, Flow rate=1 mL/min, Injection volume 10 uL, Run time 30 min, Isopropyl alcohol:Hexane=25:75, Isocratic; or (option 2) an (R,R)—Whelk—O1 chiral column, 4.6 mm×250 mm, 5 μm column, UV detection wavelength=254 nm, Flow rate=1 mL/min, Injection volume 20 uL, Run time 80 min, Isopropyl alcohol: Hexane=99.5:0.5, Isocratic. High resolution mass spectra (HRMS) were obtained by the Analytical Instrument Center at the UW-Madison School of Pharmacy on an Electron Spray Injection (ESI) mass spectrometer. Flash chromatography separations were carried out using a Teledyne Isco CombiFlash Rf 200 purification system with normal phase silica gel columns or reverse phase C-18 column. Microwave irradiated reactions were carried out using an Anton Paar Monowave 300 (Microwave synthesis reactor).

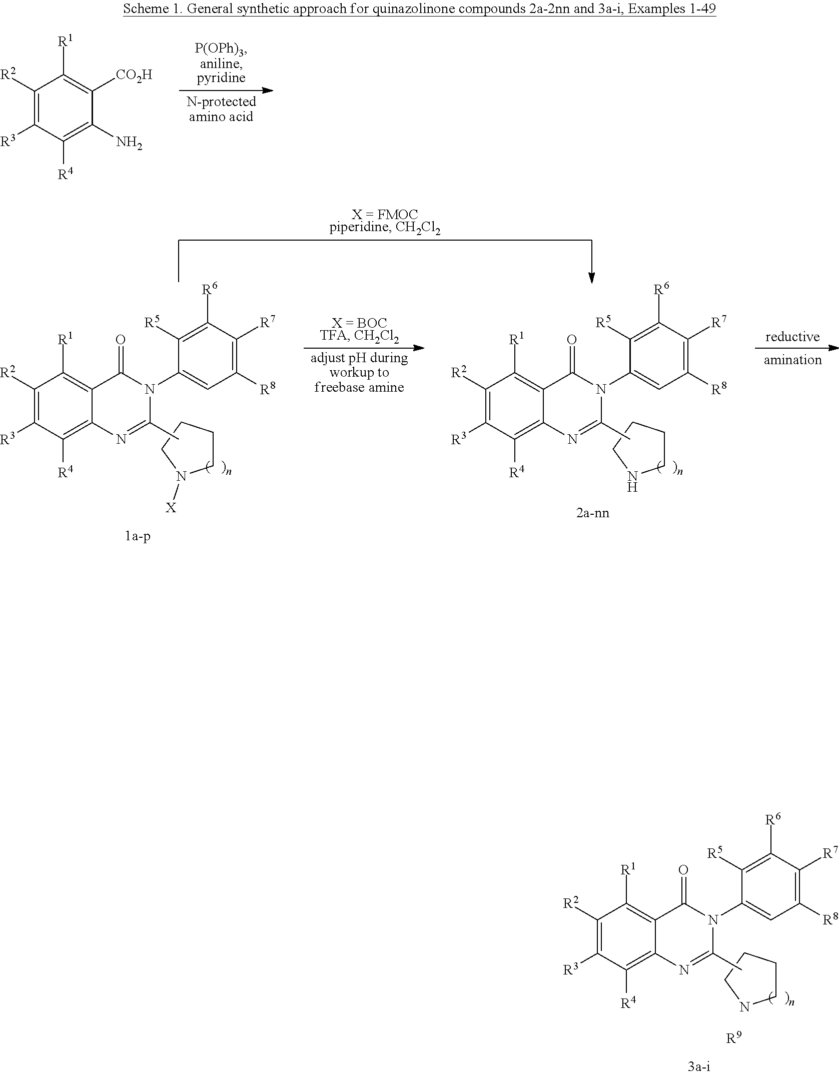

### Example 1: Synthesis of 6-nitro-3-phenyl-2-(pyrrolidin-2-yl) quinazolin-4 (3H)-one 2a)

Prepared according to Scheme 1.

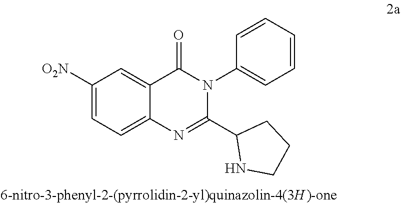 [SMILES: O=c1c2cc([N+](=O)[O-])ccc2nc(C2CCCN2)n1-c1ccccc1]

Step 1. Synthesis of tert-butyl 2-(6-nitro-4-oxo-3-phenyl-3,4-dihydroquinazolin-2-yl)pyrrolidine-1-carboxylate (1a). 5-Nitroanthranilic acid (91.00 mg, 0.50 mmol) and N-Boc-DL-proline (215.25 mg, 1.00 mmol) were dissolved in dry pyridine (3 mL). Triphenyl phosphite (0.40 mL, 1.50 mmol) was added to the mixture and the reaction mixture was heated at 100° C. with MWI for 20 min. Aniline (0.18 mL, 2.00 mmol) was added to the mixture, and the reaction mixture was heated at 100° C. with MWI for 1 min. Pyridine was removed in vacuo and the residue was diluted with CH₂Cl₂ (80 mL) and washed with 1N aqueous HCl (30 mL). The organic layer was washed sequentially with H₂O (30 mL) and brine (30 mL). The separated organic extract was dried over anhydrous Na₂SO₄, filtered, and then the solvent was evaporated under reduced pressure. The residue was purified by flash chromatography (0% Ethyl acetate/Hexane˜50% Ethyl acetate/Hexane) to afford 1a (105.1 mg, 48.2%) as pale yellow solid. ¹H NMR (500 MHz, CDCl₃) δ 9.11 (dd, J=13.7, 2.6 Hz, 1H), 8.52 (ddd, J=23.3, 9.0, 2.6 Hz, 1H), 7.78 (dd, J=25.7, 9.0 Hz, 1H), 7.58-7.35 (m, 4H), 7.23 (t, J=6.9 Hz, 1H), 4.50 (ddd, J=37.1, 7.8, 4.3 Hz, 1H), 3.83-3.66 (m, 1H), 3.57-3.41 (m, 1H), 2.15-1.88 (m, 3H), 1.86-1.74 (m, 1H), 1.43 (s, 4H), 1.29 (s, 5H). MS (ESI) m/z: 437 (M+1)⁺.

Step 2. Synthesis of 6-nitro-3-phenyl-2-(pyrrolidin-2-yl) quinazolin-4 (3H)-one (2a). Under nitrogen, trifluoroacetic acid (2.53 mL, 34.4 mmol) was added to a solution of 1a (0.75 g, 1.7 mmol) in dry CH₂Cl₂ (30 mL) at 0° C. and stirred at rt for 12 h. The solvent was removed in vacuo, and the residue was diluted with CH₂Cl₂ (100 mL), and washed sequentially with saturated solutions of Na₂CO₃ (30 mL) and brine (30 mL). The organic extract was dried over anhydrous Na₂SO₄, filtered, and then the solvent was evaporated under reduced pressure. The residue was purified by flash chromatography (0% MeOH/CH₂Cl₂˜5% MeOH/CH₂Cl₂) to afford 2a (0.52 g, 90.0%) as pale yellow solid. ¹H NMR (400 MHz, CDCl₃) δ 9.11 (d, J=2.4 Hz, 1H), 8.55 (dd, J=9.0, 2.7 Hz, 1H), 7.83 (d, J=8.9 Hz, 1H), 7.64-7.52 (m, 3H), 7.35-7.28 (m, 2H), 3.85 (t, J=7.2 Hz, 1H), 3.31-3.23 (m, 1H), 2.78 (dt, J=11.1, 6.9 Hz, 1H), 1.87-1.73 (m, 4H). MS (ESI) m/z: 337.1 (M+1)⁺.

### Example 2: Synthesis of (R)-6-nitro-3-phenyl-2-(pyrrolidin-2-yl) quinazolin-4 (3H)-one (2b)

Prepared as shown in Scheme 1.

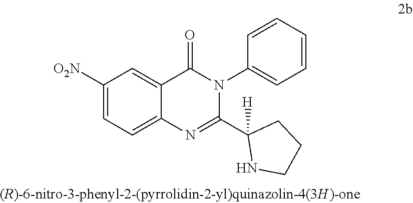 [SMILES: O=c1c2cc([N+](=O)[O-])ccc2nc([C@H]2CCCN2)n1-c1ccccc1]

Step 1. Synthesis of tert-butyl (R)-2-(6-nitro-4-oxo-3-phenyl-3,4-dihydroquinazolin-2-yl) pyrrolidine-1-carboxylate (1b). 1b (0.61 g) was prepared from 5-nitroanthranilic acid (0.55 g, 3.00 mmol), N-Boc-D-proline (1.3 g, 6.0 mmol), triphenyl phosphite (2.4 mL, 9.00 mmol) and aniline (1.1 mL, 12.0 mmol) in the same manner as described for 1a. Pale yellow solid. ¹H NMR (400 MHz, CDCl₃) δ 9.08 (dd, J=12.8, 2.6 Hz, 1H), 8.51 (ddd, J=20.0, 9.0, 2.6 Hz, 1H), 7.78 (dd, J=20.4, 9.0 Hz, 1H), 7.65-7.33 (m, 4H), 7.27-7.21 (m, 1H), 4.50 (ddd, J=28.3, 7.5, 4.5 Hz, 1H), 3.82-3.66 (m, 1H), 3.58-3.40 (m, 1H), 2.14-1.92 (m, 3H), 1.86-1.76 (m, 1H), 1.43 (s, 4H), 1.29 (s, 5H). MS (ESI) m/z: 437 (M+1)⁺.

Step 2. Synthesis of (R)-6-nitro-3-phenyl-2-(pyrrolidin-2-yl) quinazolin-4 (3H)-one (2b). 2b (0.20 g, 60.4%) was prepared from 1b (0.43 g, 0.99 mmol) and trifluoroacetic acid (1.5 mL, 19.8 mmol) in the same manner as described for 2a. Pale yellow solid. ¹H NMR (500 MHz, CDCl₃) δ 9.11 (d, J=2.6 Hz, 1H), 8.55 (dd, J=9.0, 2.6 Hz, 1H), 7.81 (d, J=8.9 Hz, 1H), 7.62-7.51 (m, 3H), 7.35-7.28 (m, 2H), 3.85 (t, J=7.1 Hz, 1H), 3.31-3.24 (m, 1H), 2.88-2.67 (m, 1H), 1.87-1.64 (m, 4H). MS (ESI) m/z: 337.1 (M+1)⁺.

### Example 3: Synthesis of (R)-6-nitro-3-phenyl-2-(1-(tetrahydro-2H-pyran-4-yl) pyrrolidin-2-yl) quinazolin-4 (3H)-one (3a)

Prepared by reduction amination of 2b as shown in Scheme 1

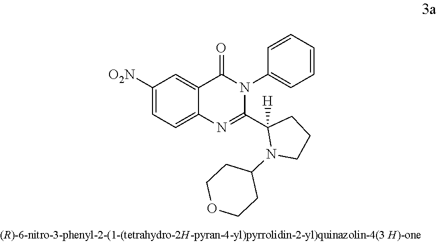 [SMILES: O=c1c2cc([N+](=O)[O-])ccc2nc([C@H]2CCCN2C2CCOCC2)n1-c1ccccc1]

### Synthesis of (R)-6-nitro-3-phenyl-2-(1-(tetrahydro-2H-pyran-4-yl) pyrrolidin-2-yl) quinazolin-4 (3H)-one (3a)

Acetic acid (0.05 mL) was added to the solution of 2b (50.0 mg, 0.15 mmol) and tetrahydro-4H-pyran-4-one (22.5 mg, 0.23 mmol) in MeOH (2 mL) and CH₂Cl₂ (2 mL) at rt. After stirring at rt for 1 h, the solution was cooled to 0° C., and sodium cyanoborohydride (14.2 mg, 0.23 mmol) was added. After 10 min, the ice bath was removed, and the reaction was allowed to stir for 12 h. Solvent was removed in vacuo and the residue was diluted with CH₂Cl₂ (20 mL), and washed sequentially with saturated solutions of Na₂CO₃ (10 mL) and brine (10 mL). The separated organic extract was dried over anhydrous Na₂SO₄, filtered, and the solvent was evaporated under reduced pressure. The residue was purified by flash chromatography (0% MeOH/CH₂Cl₂ to 10% MeOH/CH₂Cl₂) to afford 3a (42.10 mg, 66.8%) as pale yellow solid. ¹H NMR (500 MHz, CDCl₃) δ 9.07 (d, J=2.6 Hz, 1H), 8.53 (dd, J=9.0, 2.6 Hz, 1H), 7.98 (d, J=9.0 Hz, 1H), 7.63-7.56 (m, 3H), 7.26-7.20 (m, 2H), 3.96-3.91 (m, 2H), 3.56 (dd, J=8.1, 5.7 Hz, 1H), 3.36-3.32 (m, 3H), 2.78-2.75 (m, 1H), 2.59 (dd, J=16.2, 8.2 Hz, 1H), 2.07-1.91 (m, 3H), 1.82-1.75 (m, 1H), 1.65 (d, J=10.9 Hz, 1H), 1.58-1.52 (m, 3H). MS (ESI) m/z: 421.1 (M+1)⁺.

### Example 4: Synthesis of (R)-2-(1-(1-methylpiperidin-4-yl) pyrrolidin-2-yl)-6-nitro-3-phenylquinazolin-4 (3H)-one (3b)

Prepared by reduction amination of 2b as shown in Scheme 1

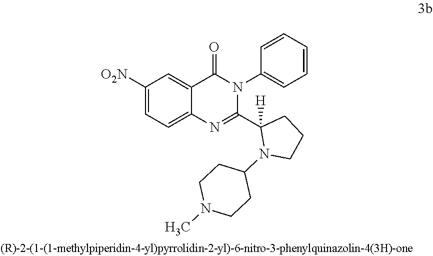 [SMILES: CN1CCC(N2CCC[C@@H]2c2nc3ccc([N+](=O)[O-])cc3c(=O)n2-c2ccccc2)CC1]

### Synthesis of (R)-2-(1-(1-methylpiperidin-4-yl) pyrrolidin-2-yl)-6-nitro-3-phenylquinazolin-4 (3H)-one (3b)

Acetic acid (0.05 mL) was added to the solution of 2b (50.0 mg, 0.15 mmol) and N-methyl-4-piperidone (25.5 mg, 0.23 mmol) in MeOH (2 mL) and CH₂Cl₂ (2 mL). The solution was stirred at rt for 1 h, then sodium cyanoborohydride (14.2 mg, 0.23 mmol) was added to the reaction mixture at 0° C. After 5 minutes, the ice bath was removed, and the reaction was stirred at rt for 12 h. The solvent was removed in vacuo, and the residue was diluted with CH₂Cl₂ (20 mL). After washing sequentially with saturated solutions of Na₂CO₃ (10 mL) and brine (10 mL), the separated organic filtrate was dried over anhydrous Na₂SO₄, filtered, and then the solvent was evaporated under reduced pressure. The residue was purified by flash chromatography (0% MeOH/CH₂Cl₂˜10% MeOH/CH₂Cl₂) to afford 3b (28.6 mg, 44.0%) as a pale yellow amorphous solid. ¹H NMR (500 MHz, CDCl₃) δ 9.09 (d, J=2.1 Hz, 1H), 8.53 (dd, J=9.0, 2.0 Hz, 1H), 7.98 (d, J=9.0 Hz, 1H), 7.59 (dd, J=11.0, 5.7 Hz, 3H), 7.25 (dt, J=16.1, 6.0 Hz, 2H), 3.58-3.51 (m, 1H), 3.29 (dd, J=11.0, 5.1 Hz, 1H), 2.85 (d, J=11.0 Hz, 2H), 2.58-2.54 (m, 2H), 2.25 (s, 3H), 1.98-1.95 (m, 5H), 1.79-1.72 (m, 2H), 1.65-1.52 (m, 3H). MS (ESI) m/z: 434.2 (M+1)⁺.

### Example 5: Synthesis of (S)-6-nitro-3-phenyl-2-(pyrrolidin-2-yl) quinazolin-4 (3H)-one (2c)

Prepared as shown in Scheme 1.

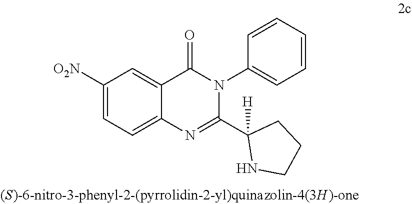 [SMILES: O=c1c2cc([N+](=O)[O-])ccc2nc([C@H]2CCCN2)n1-c1ccccc1]

Step 1. Synthesis of tert-butyl (S)-2-(6-nitro-4-oxo-3-phenyl-3,4-dihydroquinazolin-2-yl) pyrrolidine-1-carboxylate (1c). 1c (0.76 g, 43.6%) was prepared from 5-nitroanthranilic acid (0.72 g, 4.00 mmol), N-Boc-L-proline (1.7 g, 8.0 mmol), triphenyl phosphite (3.2 mL, 12.0 mmol) and aniline (1.5 mL, 16.00 mmol) in the same manner as described for 1a. Pale yellow solid. ¹H NMR (500 MHz, CDCl₃) δ 9.11 (dd, J=14.1, 2.6 Hz, 1H), 8.52 (ddd, J=23.7, 9.0, 2.6 Hz, 1H), 7.78 (dd, J=25.7, 9.0 Hz, 1H), 7.65-7.33 (m, 4H), 7.25-7.20 (m, 1H), 4.50 (ddd, J=37.1, 7.8, 4.3 Hz, 1H), 3.82-3.66 (m, 1H), 3.58-3.41 (m, 1H), 2.14-1.94 (m, 3H), 1.84-1.76 (m, 1H), 1.43 (s, 4H), 1.29 (s, 5H). MS (ESI) m/z: 437 (M+1)⁺.

Step 2. Synthesis of (S)-6-nitro-3-phenyl-2-(pyrrolidin-2-yl) quinazolin-4 (3H)-one (2c). 2c (0.40 g, 78.8%) was prepared from 1c (0.66 g, 1.51 mmol) and trifluoroacetic acid (2.26 mL, 30.26 mmol) in the same manner as described for 2a. Pale yellow solid. ¹H NMR (500 MHz, CDCl₃) δ 9.11 (d, J=2.6 Hz, 1H), 8.55 (dd, J=9.0, 2.6 Hz, 1H), 7.83 (d, J=8.9 Hz, 1H), 7.65-7.52 (m, 3H), 7.35-7.28 (m, 2H), 3.86 (t, J=7.1 Hz, 1H), 3.31-3.24 (m, 1H), 2.88-2.63 (m, 1H), 1.88-1.64 (m, 4H). MS (ESI) m/z: 337.2 (M+1)⁺.

### Example 6: Synthesis of (S)-6-nitro-3-phenyl-2-(1-(tetrahydro-2H-pyran-4-yl)pyrrolidin-2-yl)quinazolin-4 (3H)-one (3c)

Prepared by reductive amination of 2c as shown in Scheme 1

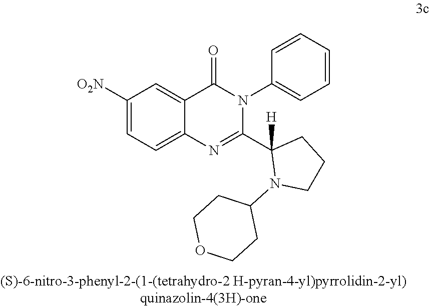 [SMILES: O=c1c2cc([N+](=O)[O-])ccc2nc([C@@H]2CCCN2C2CCOCC2)n1-c1ccccc1]

### Synthesis of (S)-6-nitro-3-phenyl-2-(1-(tetrahydro-2H-pyran-4-yl)pyrrolidin-2-yl)quinazolin-4 (3H)-one (3c)

Acetic acid (0.10 mL) was added to the solution of 2c (100.0 mg, 0.30 mmol) and tetrahydro-4H-pyran-4-one (45.1 mg, 0.45 mmol) in MeOH (4 mL) and CH₂Cl₂ (4 mL). The solution was stirred at rt for 1 h. After cooling the reaction to 0° C., sodium cyanoborohydride (28.28 mg, 0.45 mmol) was added and the reaction mixture was stirred at rt for 12 h. The solvent was removed in vacuo, and the residue was diluted with CH₂Cl₂ (40 mL) and washed sequentially with saturated solutions of Na₂CO₃ (20 mL) and brine (20 mL). The separated organic extracts were dried over anhydrous Na₂SO₄, filtered, and then the solvent was evaporated under reduced pressure. The residue was purified by flash chromatography (0% MeOH/CH₂Cl₂˜10% MeOH/CH₂Cl₂) to afford 3c (69.80 mg, 55.3%) as pale yellow solid. ¹H NMR (500 MHz, CDCl₃) δ 9.08 (d, J=2.6 Hz, 1H), 8.56 (dd, J=9.0, 2.6 Hz, 1H), 7.96 (d, J=9.0 Hz, 1H), 7.65-7.56 (m, 3H), 7.26-7.22 (m, 2H), 3.95-3.91 (m, 2H), 3.55 (dd, J=8.1, 5.7 Hz, 1H), 3.35-3.31 (m, 3H), 2.78-2.72 (m, 1H), 2.62 (dd, J=16.2, 8.2 Hz, 1H), 2.08-1.91 (m, 3H), 1.85-1.75 (m, 1H), 1.66 (d, J=10.9 Hz, 1H), 1.56-1.52 (m, 3H). MS (ESI) m/z: 421.1 (M+1)⁺.

### Example 7: Synthesis of (S)-2-(1-(1-methylpiperidin-4-yl)pyrrolidin-2-yl)-6-nitro-3-phenylquinazolin-4 (3H)-one (3d)

Prepared by reductive amination of 2c as shown in Scheme 1

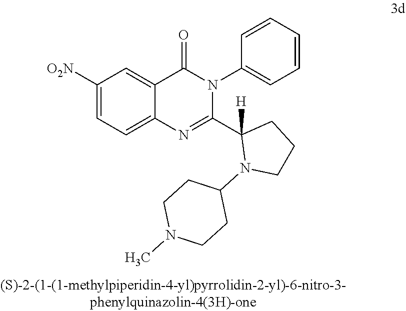 [SMILES: CN1CCC(N2CCC[C@H]2c2nc3ccc([N+](=O)[O-])cc3c(=O)n2-c2ccccc2)CC1]

### Synthesis of (S)-2-(1-(1-methylpiperidin-4-yl)pyrrolidin-2-yl)-6-nitro-3-phenylquinazolin-4 (3H)-one (3d)

Acetic acid (0.05 mL) was added to the solution of 2c (50.0 mg, 0.15 mmol) and N-methyl-4-piperidone (25.5 mg, 0.23 mmol) in MeOH (2 mL) and CH₂Cl₂ (2 mL). The solution was stirred at rt for 1 h, before sodium cyanoborohydride (14.16 mg, 0.23 mmol) was added to the reaction mixture at 0° C. After stirring at rt overnight, the solvent was removed in vacuo and the residue was diluted with CH₂Cl₂ (20 mL). After washing sequentially with saturated solutions of Na₂CO₃ (10 mL) and brine (10 mL), the separated organic extract was dried over anhydrous Na₂SO₄, filtered, and the solvent was evaporated under reduced pressure. The residue was purified by flash chromatography (0% MeOH/CH₂Cl₂ to 10% MeOH/CH₂Cl₂) to afford 3d (26.50 mg, 40.8%) as a pale yellow amorphous solid. ¹H NMR (500 MHz, CDCl₃) δ 9.06 (d, J=2.1 Hz, 1H), 8.55 (dd, J=9.0, 2.0 Hz, 1H), 7.95 (d, J=9.0 Hz, 1H), 7.56 (dd, J=11.0, 5.7 Hz, 3H), 7.25 (dt, J=16.1, 6.0 Hz, 2H), 3.62-3.55 (m, 1H), 3.25 (dd, J=11.0, 5.1 Hz, 1H), 2.86 (d, J=11.0 Hz, 2H), 2.58-2.52 (m, 2H), 2.32 (s, 3H), 1.96-1.92 (m, 5H), 1.78-1.72 (m, 2H), 1.65-1.55 (m, 3H). MS (ESI) m/z: 434.2 (M+1)⁺.

### Example 8: Synthesis of 3-(4-methoxyphenyl)-6-nitro-2-(pyrrolidin-2-yl) quinazolin-4 (3H)-one (2d)

Prepared as shown in Scheme 1

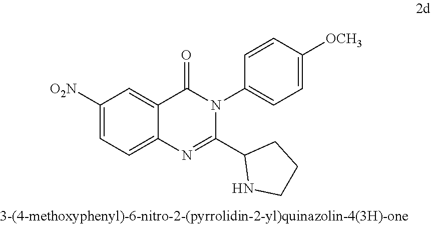 [SMILES: COc1ccc(-n2c(C3CCCN3)nc3ccc([N+](=O)[O-])cc3c2=O)cc1]

Step 1. Synthesis of tert-butyl 2-(3-(4-methoxyphenyl)-6-nitro-4-oxo-3,4-dihydroquinazolin-2-yl) pyrrolidine-1-carboxylate (1d). 1d (0.27 g) was prepared from 5-nitroanthranilic acid (0.54 g, 3.00 mmol), N-Boc-DL-proline (1.3 g, 6.00 mmol), triphenyl phosphite (2.36 mL, 9.00 mmol) and 4-methoxyaniline (1.46 g, 12.00 mmol) in the same manner as described for 1a. Pale yellow solid. ¹H NMR (500 MHz, CDCl₃) δ 9.10 (dd, J=13.3, 2.5 Hz, 1H), 8.51 (ddd, J=23.0, 9.0, 2.6 Hz, 1H), 7.77 (dd, J=24.7, 9.0 Hz, 1H), 7.51-7.20 (m, 1H), 7.14-7.03 (m, 3H), 4.66-4.43 (m, 1H), 3.89 (d, J=10.8 Hz, 3H), 3.80-3.67 (m, 1H), 3.59-3.37 (m, 1H), 2.09-1.82 (m, 4H), 1.43 (s, 4H), 1.27 (s, 5H). MS (ESI) m/z: 467 (M+1)⁺.

Step 2. Synthesis of 3-(4-methoxyphenyl)-6-nitro-2-(pyrrolidin-2-yl)quinazolin-4 (3H)-one (2d)

Under nitrogen, trifluoroacetic acid (0.83 mL, 11.20 mmol) was added to a solution of 1d (0.26 g, 0.56 mmol) in dry CH₂Cl₂ (10 mL) at 0° C., and the resulting reaction mixture was stirred at rt overnight. After removing the solvent in vacuo, the residue was diluted with CH₂Cl₂ (100 mL), washed sequentially with saturated solutions of Na₂CO₃ (30 mL) and brine (30 mL), and the separated organic extracts were dried over anhydrous Na₂SO₄, filtered, and the solvent was evaporated under reduced pressure. The residue was purified by flash chromatography (0% MeOH/CH₂Cl₂˜5% MeOH/CH₂Cl₂) to afford 2d (136.0 mg, 66.3%) as pale yellow solid. ¹H NMR (500 MHz, CDCl₃) δ 9.11 (d, J=2.6 Hz, 1H), 8.54 (dd, J=9.0, 2.6 Hz, 1H), 7.81 (d, J=9.0 Hz, 1H), 7.24-7.15 (m, 2H), 7.10-7.01 (m, 2H), 3.94-3.85 (m, 4H), 3.35-3.22 (m, 1H), 2.79 (dt, J=11.0, 6.7 Hz, 1H), 1.84-1.69 (m, 4H). MS (ESI) m/z: 367.1 (M+1)⁺.

### Example 9: Synthesis of (R)-3-(4-methoxyphenyl)-6-nitro-2-(pyrrolidin-2-yl) quinazolin-4 (3H)-one (2e)

Prepared as shown in Scheme 1.

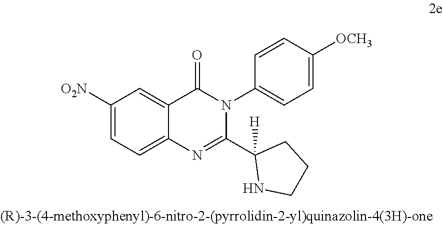 [SMILES: COc1ccc(-n2c([C@H]3CCCN3)nc3ccc([N+](=O)[O-])cc3c2=O)cc1]

Step 1. Synthesis of tert-butyl (R) 2-(3-(4-methoxyphenyl)-6-nitro-4-oxo-3,4-dihydroquinazolin-2-yl) pyrroli-dine-1-carboxylate (1e). 1e (0.43 g) was prepared from 5-nitroanthranilic acid (0.36 g, 2.00 mmol), N-Boc-D-proline (0.86 g, 4.00 mmol), triphenyl phosphite (1.60 mL, 6.00 mmol) and 4-methoxyaniline (0.98 g, 8.00 mmol) in the same manner as described for 1a. Pale yellow solid. ¹H NMR (500 MHz, CDCl₃) δ 9.07 (dd, J=12.8, 2.6 Hz, 1H), 8.52 (ddd, J=23.0, 9.0, 2.6 Hz, 1H), 7.77 (dd, J=24.6, 9.0 Hz, 1H), 7.54-7.21 (m, 1H), 7.16-7.03 (m, 3H), 4.66-4.43 (m, 1H), 3.89 (d, J=10.9 Hz, 3H), 3.80-3.66 (m, 1H), 3.58-3.36 (m, 1H), 2.09-1.84 (m, 4H), 1.44 (s, 4H), 1.28 (s, 5H). MS (ESI) m/z: 467 (M+1)⁺.

Step 2. Synthesis of (R)-3-(4-methoxyphenyl)-6-nitro-2-(pyrrolidin-2-yl)quinazolin-4 (3H)-one (2e)

Under nitrogen, trifluoroacetic acid (1.26 mL, 17.16 mmol) was added to a solution of 1e (0.40 g, 0.85 mmol) in dry CH₂Cl₂ (15 mL) at 0° C. After stirring at rt overnight, the solvent was removed in vacuo and the residue was diluted with CH₂Cl₂ (100 mL), and washed with saturated solutions of Na₂CO₃ (40 mL) and brine (40 mL). The separated organic extracts were dried over anhydrous Na₂SO₄, filtered, and then the solvent was evaporated under reduced pressure. The residue was purified by flash chromatography (0% MeOH/CH₂Cl₂˜5% MeOH/CH₂Cl₂) to afford 2e (0.20 g, 64.3%) as pale yellow solid. ¹H NMR (500 MHz, CDCl₃) δ 9.11 (d, J=2.6 Hz, 1H), 8.56 (dd, J=9.0, 2.6 Hz, 1H), 7.81 (d, J=9.0 Hz, 1H), 7.23-7.15 (m, 2H), 7.09-7.04 (m, 2H), 3.94-3.83 (m, 4H), 3.34-3.22 (m, 1H), 2.79 (dt, J=11.1, 6.8 Hz, 1H), 1.85-1.67 (m, 4H). MS (ESI) m/z: 367.1 (M+1)⁺.

### Example 10: Synthesis of (R)-3-(4-methoxyphenyl)-2-(1-methylpyrrolidin-2-yl)-6-nitroquinazolin-4 (3H)-one (3e)

Prepared via reductive amination of 2e as shown in Scheme 1

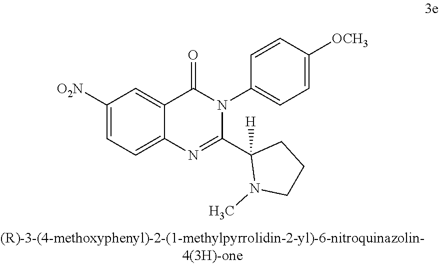 [SMILES: COc1ccc(-n2c([C@H]3CCCN3C)nc3ccc([N+](=O)[O-])cc3c2=O)cc1]

### Synthesis of (R)-3-(4-methoxyphenyl)-2-(1-methylpyrrolidin-2-yl)-6-nitroquinazolin-4 (3H)-one (3e)

Acetic acid (0.03 mL) was added to the solution of 2e (100.0 mg, 0.27 mmol) and formaldehyde 37% w/w aqueous (44.63 mg, 0.55 mmol) in MeOH (3 mL) and CH₂Cl₂ (3 mL). After stirring at rt for 1 h, sodium cyanoborohydride (25.80 mg, 0.41 mmol) was added to the reaction mixture at 0° C. The ice bath was removed, and the reaction was stirred at rt overnight. Solvent was removed in vacuo, and the residue was diluted with CH₂Cl₂ (10 mL) and washed sequentially with saturated solutions of Na₂CO₃ (10 mL) and brine (10 mL). The separated organic extracts were dried over anhydrous Na₂SO₄, filtered, and the solvent was evaporated under reduced pressure. The residue was purified by flash chromatography (0% MeOH/CH₂Cl₂˜5% MeOH/CH₂Cl₂) to afford 3e (63.7 mg, 62.0%) as a pale yellow amorphous solid. ¹H NMR (500 MHz, CDCl₃) δ 9.11 (d, J=2.6 Hz, 1H), 8.54 (dd, J=9.0, 2.7 Hz, 1H), 8.05 (d, J=9.0 Hz, 1H), 7.18-7.12 (m, 4H), 3.91 (s, 3H), 3.29-3.26 (m, 1H), 3.09-3.06 (m, 1H), 2.35 (s, 3H), 2.23-2.14 (m, 1H), 2.05-1.85 (m, 3H), 1.68-1.64 (m, 1H). MS (ESI) m/z: 381.1 (M+1)⁺.

### Example 11: Synthesis of (R)-3-(4-methoxyphenyl)-6-nitro-2-(1-(oxetan-3-yl)pyrrolidin-2-yl) quinazolin-4 (3H)-one (3f)

Prepared via reductive amination of 2e as shown in Scheme 1

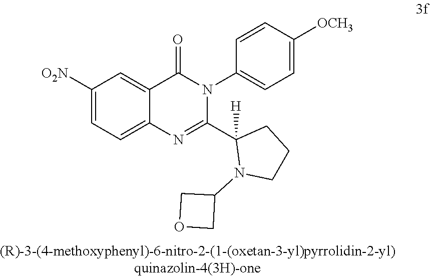 [SMILES: COc1ccc(-n2c([C@H]3CCCN3C3COC3)nc3ccc([N+](=O)[O-])cc3c2=O)cc1]

### Synthesis of (R)-3-(4-methoxyphenyl)-6-nitro-2-(1-(oxetan-3-yl) pyrrolidin-2-yl) quinazolin-4 (3H)-one (3f)

Acetic acid (0.05 mL) was added to the solution of 2e (50.0 mg, 0.16 mmol) and tetrahydro-4H-pyran-4-one (18.0 mg, 0.25 mmol) in MeOH (3 mL) and CH₂Cl₂ (3 mL). After stirring at rt for 1 h, sodium cyanoborohydride (15.7 mg, 0.25 mmol) was added to the reaction mixture at 0° C. and stirred for 5 minutes. The ice bath was removed, and the reaction was stirred at rt overnight. Solvent was removed in vacuo, and the residue was diluted with CH₂Cl₂ (20 mL). After washing sequentially with saturated solutions of Na₂CO₃ (10 mL) and brine (10 mL), the separated organic extract was dried over anhydrous Na₂SO₄, filtered, and the solvent was evaporated under reduced pressure. The residue was purified by flash chromatography (0% MeOH/CH₂Cl₂˜5% MeOH/CH₂Cl₂) to afford 3f (16.60 mg, 24.6%) as a pale yellow amorphous solid. ¹H NMR (500 MHz, CDCl₃) δ 9.10 (d, J=2.6 Hz, 1H), 8.54 (dd, J=9.0, 2.7 Hz, 1H), 7.94 (d, J=9.0 Hz, 1H), 7.14-7.06 (m, 4H), 4.75 (t, J=6.6 Hz, 1H), 4.64 (t, J=6.6 Hz, 1H), 4.53 (t, J=6.9 Hz, 2H), 4.06-3.98 (m, 1H), 3.90 (s, 3H), 3.55 (t, J=7.3 Hz, 1H), 3.42-3.33 (m, 1H), 2.65-2.62 (m, 1H), 2.08-1.96 (m, 2H), 1.86-1.75 (m, 1H). MS (ESI) m/z: 423.1 (M+1)⁺.

### Example 12: Synthesis of (S)-3-(4-methoxyphenyl)-6-nitro-2-(pyrrolidin-2-yl) quinazolin-4 (3H)-one (2f)

Prepared as shown in Scheme 1.

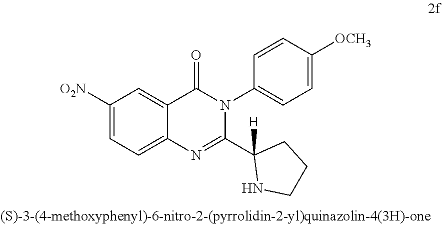 [SMILES: COc1ccc(-n2c([C@@H]3CCCN3)nc3ccc([N+](=O)[O-])cc3c2=O)cc1]

Step 1. Synthesis of tert-butyl (S) 2-(3-(4-methoxyphenyl)-6-nitro-4-oxo-3,4-dihydroquinazolin-2-yl) pyrroli-dine-1-carboxylate (1f). 1f (0.32 g) was prepared from 5-nitroanthranilic acid (0.36 g, 2.00 mmol), N-Boc-L-proline (0.86 g, 4.00 mmol), triphenyl phosphite (1.60 mL, 6.00 mmol) and 4-methoxyaniline (0.98 g, 8.00 mmol) in the same manner as described for 1a. Pale yellow solid. ¹H NMR (500 MHz, CDCl₃) δ 9.06 (dd, J=12.9, 2.6 Hz, 1H), 8.54 (ddd, J=22.8, 9.0, 2.6 Hz, 1H), 7.76 (dd, J=24.6, 9.0 Hz, 1H), 7.55-7.22 (m, 1H), 7.16-7.04 (m, 3H), 4.68-4.42 (m, 1H), 3.89 (d, J=10.9 Hz, 3H), 3.84-3.66 (m, 1H), 3.56-3.36 (m, 1H), 2.09-1.86 (m, 4H), 1.43 (s, 4H), 1.26 (s, 5H). MS (ESI) m/z: 467 (M+1)⁺.

Step 2. Synthesis of (S)-3-(4-methoxyphenyl)-6-nitro-2-(pyrrolidin-2-yl) quinazolin-4 (3H)-one (2f)

Under nitrogen, trifluoroacetic acid (0.82 mL, 11.15 mmol) was added to a solution of 1f (0.26 g, 0.56 mmol) in dry CH₂Cl₂ (10 mL) at 0° C. The solution was stirred at rt overnight, and then the solvent was removed in vacuo. The residue was diluted with CH₂Cl₂ (100 mL), washed sequentially with saturated solutions of Na₂CO₃ (30 mL) and brine (30 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 2f (0.12 g, 58.5%) as pale yellow solid. ¹H NMR (500 MHz, CDCl₃) δ 9.11 (d, J=2.6 Hz, 1H), 8.54 (dd, J=9.0, 2.6 Hz, 1H), 7.81 (d, J=9.0 Hz, 1H), 7.23-7.16 (m, 2H), 7.09-7.04 (m, 2H), 3.93-3.87 (m, 4H), 3.31-3.23 (m, 1H), 2.79 (dt, J=11.2, 6.7 Hz, 1H), 1.85-1.67 (m, 4H). MS (ESI) m/z: 367.1 (M+1)⁺.

### Example 13: Synthesis of (S)-3-(4-methoxyphenyl)-2-(1-methylpyrrolidin-2-yl)-6-nitroquinazolin-4 (3H)-one (3g)

Prepared via reductive amination of 2f as shown in Scheme 1

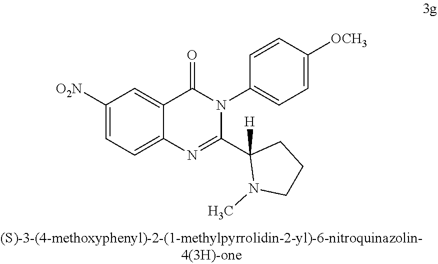 [SMILES: COc1ccc(-n2c([C@@H]3CCCN3C)nc3ccc([N+](=O)[O-])cc3c2=O)cc1]

### Synthesis of (S)-3-(4-methoxyphenyl)-2-(1-methylpyrrolidin-2-yl)-6-nitroquinazolin-4 (3H)-one (3g)

Acetic acid (0.03 mL) was added to the solution of 2f (60.00 mg, 0.16 mmol) and formaldehyde 37% w/w aqueous (26.60 mg, 0.32 mmol) in MeOH (3 mL) and CH₂Cl₂ (3 mL). After stirring at rt for 1 h, sodium cyanoborohydride (15.1 mg, 0.24 mmol) was added to the reaction mixture at 0° C. The solution was stirred at rt overnight, and then the solvent was removed in vacuo. The residue was diluted with CH₂Cl₂ (10 mL), washed sequentially with saturated solutions of Na₂CO₃ (10 mL) and brine (50 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 3g (40.70 mg, 66.8%) as a pale yellow amorphous solid. ¹H NMR (500 MHz, CDCl₃) δ 9.12 (d, J=2.6 Hz, 1H), 8.56 (dd, J=9.0, 2.7 Hz, 1H), 8.04 (d, J=9.0 Hz, 1H), 7.21-7.12 (m, 4H), 3.93 (s, 3H), 3.28-3.26 (m, 1H), 3.12-3.06 (m, 1H), 2.36 (s, 3H), 2.24-2.14 (m, 1H), 2.06-1.85 (m, 3H), 1.66-1.64 (m, 1H). MS (ESI) m/z: 381.1 (M+1)⁺.

### Example 14: Synthesis of (R)-3-(4-methoxyphenyl)-4-oxo-2-(pyrrolidin-2-yl)-3,4-dihydroquinazoline-6-carbonitrile (2g)

Prepared as shown in Scheme 1.

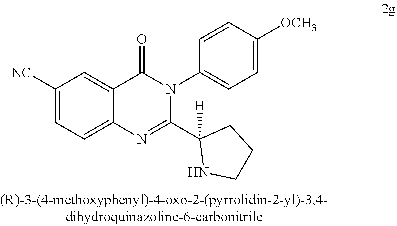 [SMILES: COc1ccc(-n2c([C@H]3CCCN3)nc3ccc(C#N)cc3c2=O)cc1]

Step 1. Synthesis of tert-butyl (R)-2-(6-cyano-3-(4-methoxyphenyl)-4-oxo-3,4-dihydroquinazolin-2-yl)pyrrolidine-1-carboxylate (1g). 1g (0.31 g) was prepared from 2-amino-5-cyanobenzoic acid (0.24 g, 1.50 mmol), N-Boc-D-proline (0.65 g, 3.00 mmol), triphenyl phosphite (1.2 mL, 4.50 mmol) and 4-methoxyaniline (0.73 g, 6.00 mmol) in the same manner as described for 1a. Pale yellow solid. ¹H NMR (500 MHz, CDCl3) δ 8.57 (dd, J=13.1, 1.3 Hz, 1H), 7.91 (ddd, J=24.3, 8.5, 1.6 Hz, 1H), 7.73 (dd, J=22.6, 8.5 Hz, 1H), 7.48-7.18 (m, 1H), 7.14-7.02 (m, 3H), 4.58-4.52 (m, 1H), 3.89 (d, J=10.8 Hz, 3H), 3.78-3.66 (m, 1H), 3.57-3.41 (m, 1H), 2.09-1.82 (m, 4H), 1.43 (s, 4H), 1.27 (s, 5H). MS (ESI) m/z: 447 (M+1)⁺.

Step 2. Synthesis of (R)-3-(4-methoxyphenyl)-4-oxo-2-(pyrrolidin-2-yl)-3,4-dihydroquinazoline-6-carbonitrile (2g)

Under nitrogen, trifluoroacetic acid (0.49 mL, 6.72 mmol) was added to a solution of 1g (0.15 g, 0.34 mmol) in dry CH₂Cl₂ (10 mL) at 0° C. The solution was stirred at rt overnight, and then the solvent was removed in vacuo. The residue was diluted with CH₂Cl₂ (100 mL), washed sequentially with saturated solutions of Na₂CO₃ (40 mL) and brine (40 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 2g (65.60 mg, 56.5%) as pale yellow solid. ¹H NMR (500 MHz, CDCl₃) δ 8.56 (d, J=1.8 Hz, 1H), 7.93 (dd, J=8.5, 1.9 Hz, 1H), 7.77 (d, J=8.5 Hz, 1H), 7.22-7.14 (m, 2H), 7.08-7.02 (m, 2H), 3.91-3.85 (m, 4H), 3.31-3.21 (m, 1H), 2.87 (s, 1H), 2.84-2.74 (m, 1H), 1.82-1.69 (m, 4H). MS (ESI) m/z: 347.1 (M+1)⁺.

### Example 15: Synthesis of (S)-3-(4-methoxyphenyl)-4-oxo-2-(pyrrolidin-2-yl)-3,4-dihydroquinazoline-6-carbonitrile (2h)

Prepared as shown in Scheme 1.

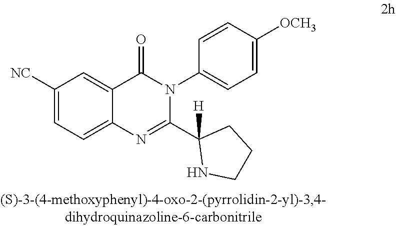 [SMILES: COc1ccc(-n2c([C@@H]3CCCN3)nc3ccc(C#N)cc3c2=O)cc1]

Step 1. Synthesis of tert-butyl (S)-2-(6-cyano-3-(4-methoxyphenyl)-4-oxo-3,4-dihydroquinazolin-2-yl) pyrrolidine-1-carboxylate (1 h). 1 h (0.34 g) was prepared from 2-amino-5-cyanobenzoic acid (0.24 g, 1.50 mmol), N-Boc-D-proline (0.65 g, 3.00 mmol), triphenyl phosphite (1.18 mL, 4.50 mmol) and 4-methoxyaniline (0.73 g, 6.00 mmol) in the same manner as described for 1a. Pale yellow solid. ¹H NMR (500 MHz, CDCl₃) δ 8.56 (dd, J=13.4, 1.3 Hz, 1H), 7.93 (ddd, J=24.1, 8.6, 1.6 Hz, 1H), 7.76 (dd, J=22.4, 8.6 Hz, 1H), 7.51-7.16 (m, 1H), 7.14-7.02 (m, 3H), 4.56-4.52 (m, 1H), 3.88 (d, J=10.8 Hz, 3H), 3.81-3.68 (m, 1H), 3.57-3.46 (m, 1H), 2.09-1.82 (m, 4H), 1.43 (s, 4H), 1.27 (s, 5H). MS (ESI) m/z: 447 (M+1)⁺.

Step 2. Synthesis of (S)-3-(4-methoxyphenyl)-4-oxo-2-(pyrrolidin-2-yl)-3,4-dihydroquinazoline-6-carbonitrile. Under nitrogen, trifluoroacetic acid (0.49 mL, 6.72 mmol) was added to a solution of 2 h (0.15 g, 0.34 mmol) in dry CH₂Cl₂ (10 mL) at 0° C. The solution was stirred at rt overnight, and then the solvent was removed in vacuo. The residue was diluted with CH₂Cl₂ (100 mL), washed sequentially with saturated solutions of Na₂CO₃ (40 mL) and brine (40 mL), and the separated organic extract was dried over anhydrous Na₂SO₄. Once filtered, the solvent was evaporated under reduced pressure. The residue was purified by flash chromatography (0% MeOH/CH₂Cl₂ to 5% MeOH/CH₂Cl₂) to afford 2 h (72.60 mg, 62.5%) as pale yellow solid. ¹H NMR (500 MHz, CDCl₃) δ 8.56 (d, J=1.7 Hz, 1H), 7.93 (dd, J=8.5, 1.9 Hz, 1H), 7.76 (d, J=9.7 Hz, 1H), 7.24-7.16 (m, 2H), 7.10-7.02 (m, 2H), 3.95-3.86 (m, 4H), 3.31-3.14 (m, 1H), 2.83-2.74 (m, 1H), 1.84-1.63 (m, 4H). MS (ESI) m/z: 347.1 (M+1)⁺.

### Example 16: Synthesis of (R)-6,7,8-trifluoro-3-(4-methoxyphenyl)-2-(pyrrolidin-2-yl)quinazolin-4 (3H)-one (2i)

Prepared as shown in Scheme 1.

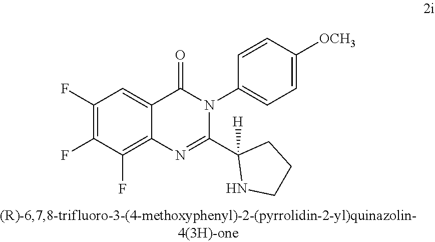 [SMILES: COc1ccc(-n2c([C@H]3CCCN3)nc3c(F)c(F)c(F)cc3c2=O)cc1]

### Synthesis of (R)-6,7,8-trifluoro-3-(4-methoxyphenyl)-2-(pyrrolidin-2-yl)quinazolin-4 (3H)-one (2i)

2-Amino-3,4,5-trifluorobenzoic acid (191.1 mg, 1.00 mmol) and Boc-D-proline (430.50 mg, 2.00 mmol) were dissolved in dry pyridine (6 mL). Triphenyl phosphite (0.78 mL, 3.00 mmol) was added to the mixture and the reaction mixture was heated at 100° C. with MWI for 40 min. 4-Methoxyaniline (0.49 g, 4.00 mmol) was added to the mixture and the reaction mixture was heated at 100° C. with MWI for 1 min. Pyridine was removed in vacuo and the residue was diluted with CH₂Cl₂ (160 mL) and washed with 1N aqueous HCl (60 mL). The organic layer was washed with H₂O (60 mL) and then brine (60 mL). After separating the organic extract, it was dried over anhydrous Na₂SO₄, filtered, then the solvent was evaporated under reduced pressure. The residue was purified by flash chromatography (0% ethyl acetate/hexane˜30% ethyl acetate/hexane) to remove most of impurities and pyridine to afford the crude product (0.12 g) as a colorless oil.

Under nitrogen, trifluoroacetic acid (0.36 mL, 5.00 mmol) was added to a solution of the crude colorless oil described above in dry CH₂Cl₂ (6 mL) at 0° C. The solution was allowed to stir at rt overnight. Solvent was removed in vacuo, and the residue was diluted with CH₂Cl₂ (60 mL), washed sequentially with saturated solutions of Na₂CO₃ (30 mL) and brine (30 mL). The separated organic extract was dried over anhydrous Na₂SO₄, filtered, and then the solvent was evaporated under reduced pressure. The residue was purified by flash chromatography (0% MeOH/CH₂Cl₂˜5% MeOH/CH₂Cl₂) to afford 2i (38.60 mg, 10.3%) as off-white solid. ¹H NMR (500 MHz, CDCl₃) δ 7.84 (t, J=8.1 Hz, 1H), 7.22-7.18 (m, 1H), 7.15 (d, J=9.7 Hz, 1H), 7.05 (d, J=8.8 Hz, 2H), 3.95-3.81 (m, 4H), 3.31-3.25 (m, 1H), 2.81-2.75 (m, 1H), 2.65 (brs, 1H), 1.86-1.68 (m, 4H). MS (ESI) m/z: 376.1 (M+1)⁺.

### Example 17: Synthesis of (R)-3-(3-azidophenyl)-6-nitro-2-(pyrrolidin-2-yl) quinazolin-4 (3H)-one (2j)

Prepared according to Scheme 1.

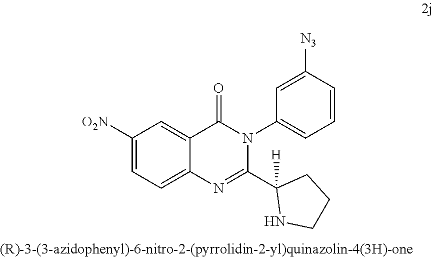 [SMILES: [N-]=[N+]=Nc1cccc(-n2c([C@H]3CCCN3)nc3ccc([N+](=O)[O-])cc3c2=O)c1]

Step 1. Synthesis of 3-azidoaniline. To a three-necked flask was added 3-iodoaniline (1.00 g, 4.56 mmol), sodium azide (0.60 g, 9.13 mmol), sodium ascorbate (45.56 mg, 0.23 mmol) and DMSO:H₂O (4:1, 10 mL). The reaction mixture was degassed and flushed with nitrogen for 5 min, and under nitrogen gas atmosphere, copper iodide (86.85 mg, 0.45 mmol) was added into the reaction mixture, followed by addition of N, N′-dimethylethylenediamine (60.00 mg, 0.68 mmol). The reaction mixture was stirred at rt for 4 h. After reaction was complete, the reaction mixture was treated with water (100 mL) and extracted with ethyl acetate (3×100 mL). The combined organic layers were sequentially washed with water (100 mL) and brine (100 mL). The separated organic extract was dried over anhydrous Na₂SO₄, filtered, and then the solvent was evaporated under reduced pressure. The residue was purified by flash chromatography (CH₂Cl₂) to afford 3-azidoaniline (0.41 g, 67.1%) as pale yellow solid. ¹H NMR (500 MHz, CDCl₃) δ 7.11 (t, J=8.0 Hz, 1H), 6.44 (dt, J=7.9, 2.3 Hz, 2H), 6.31 (t, J=2.1 Hz, 1H), 3.69 (brs, 2H). MS (ESI) m/z: 135.1 (M+1)⁺.

Step 2. Synthesis of tert-butyl (R)-2-(3-(3-azidophenyl)-6-nitro-4-oxo-3,4-dihydroquinazolin-2-yl) pyrrolidine-1-carboxylate (1i). 5-Nitroanthranilic acid (91.1 mg, 0.5 mmol) and Boc-D-proline (215.25 mg, 1.00 mmol) were dissolved in dry pyridine (3 mL). Triphenyl phosphite (0.39 mL, 1.50 mmol) was added to the mixture and the reaction mixture was heated at 100° C. with MWI for 40 min. The 3-azidoaniline (268.48 mg, 2.00 mmol) was added, and the reaction mixture was heated at 100° C. with MWI for 1 min. Pyridine was removed in vacuo and the residue was diluted with CH₂Cl₂ (160 mL) and washed with 1N aqueous HCl (60 mL). The organic layer was washed with H₂O (60 mL) and brine (60 mL). The separated organic extract was dried over anhydrous Na₂SO₄, filtered, and then the solvent was evaporated under reduced pressure. The residue was purified by flash chromatography (0% Ethyl acetate/CH₂Cl₂˜5% Ethyl acetate/CH₂Cl₂) to afford 1i (45.60 mg, 19.1%) as pale yellow oil. ¹H NMR (500 MHz, CDCl₃) δ 9.12-9.06 (m, 1H), 8.52 (dd, J=25.0, 8.8 Hz, 1H), 7.84-7.72 (m, 1H), 7.65-7.51 (m, 1H), 7.45-7.21 (m, 1H), 7.22-7.11 (m, 1H), 7.05-6.86 (m, 1H), 4.56-4.48 (m, 1H), 3.81-3.66 (m, 1H), 3.61-3.41 (m, 1H), 2.19-1.79 (m, 4H), 1.42 (d, J=4.5 Hz, 4H), 1.28 (d, J=4.9 Hz, 5H). MS (ESI) m/z: 478.1 (M+1)⁺.

Step 3. Synthesis of (R)-3-(3-azidophenyl)-6-nitro-2-(pyrrolidin-2-yl) quinazolin-4 (3H)-one (2j)

Under nitrogen, trifluoroacetic acid (0.12 mL, 1.60 mmol) was added to a solution of 1i (40.00 mg, 0.08 mmol) in dry CH₂Cl₂ (3 mL) at 0° C. The solution was stirred at rt overnight, and then the solvent was removed in vacuo. The residue was diluted with CH₂Cl₂ (30 mL), washed sequentially with saturated solutions of Na₂CO₃ (10 mL) and brine (10 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 2j (13.60 mg, 45.0%) as pale yellow solid. ¹H NMR (500 MHz, CDCl₃) δ 9.14 (s, 1H), 8.60 (dd, J=8.9, 2.2 Hz, 1H), 7.87 (d, J=8.9 Hz, 1H), 7.62 (td, J=8.0, 2.8 Hz, 1H), 7.27 (t, J=9.6 Hz, 1H), 7.12 (dd, J=27.9, 7.8 Hz, 1H), 7.05-6.96 (m, 1H), 3.90 (t, J=5.8 Hz, 1H), 3.36-3.31 (m, 1H), 2.88-2.81 (m, 1H), 2.63 (brs, 1H), 1.95-1.78 (m, 4H). MS (ESI) m/z: 378.1 (M+1)⁺.

### Example 18: Synthesis of (S)-3-(4-methoxyphenyl)-6-nitro-2-(pyrrolidin-3-yl) quinazolin-4 (3H)-one (2k)

Prepared according to Scheme 1

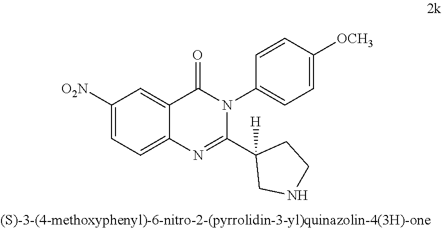 [SMILES: COc1ccc(-n2c([C@H]3CCNC3)nc3ccc([N+](=O)[O-])cc3c2=O)cc1]

Step 1. Synthesis of tert-butyl (S)-3-(3-(4-methoxyphenyl)-6-nitro-4-oxo-3,4-dihydroquinazolin-2-yl) pyrro-lidine-1-carboxylate (1j). 1j (94.10 mg, 40.3 mmol) was prepared from 5-nitroanthranilic acid (91.00 mg, 0.50 mmol), (S)—N-Boc-pyrrolidine-3-carboxylic acid (215.25 mg, 1.00 mmol), triphenyl phosphite (0.40 mL, 1.50 mmol) and 4-methoxyaniline (246.3 mg, 2.0 mmol) in the same manner as described for 1a. Pale yellow solid. ¹H NMR (400 MHz, CDCl₃) δ 9.10 (d, J=3.0 Hz, 1H), 8.54 (dd, J=9.0, 2.6 Hz, 1H), 7.84-7.73 (m, 1H), 7.22-7.15 (m, 2H), 7.12-7.06 (m, 2H), 3.90 (s, 3H), 3.75-3.66 (m, 2H), 3.58-3.55 (m, 1H), 3.30-3.20 (m, 1H), 3.19-3.16 (m, 1H), 2.46-2.30 (m, 1H), 2.06-2.04 (m, 1H), 1.45 (s, 9H). MS (ESI) m/z: 467 (M+1)⁺.

Step 2. Synthesis of (S)-3-(4-methoxyphenyl)-6-nitro-2-(pyrrolidin-3-yl) quinazolin-4 (3H)-one (2k)

Under nitrogen, trifluoroacetic acid (0.23 mL, 3.20 mmol) was added to a solution of 1j (72.60 mg, 0.16 mmol) in dry CH₂Cl₂ (3 mL) at 0° C. The solution was stirred at rt overnight, and then the solvent was removed in vacuo. The residue was diluted with CH₂Cl₂ (40 mL), washed sequentially with saturated solutions of Na₂CO₃ (15 mL) and brine (15 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 2k (21.60 mg, 36.9%) as pale yellow solid. ¹H NMR (400 MHz, CDCl₃) δ 9.08 (d, J=2.6 Hz, 1H), 8.52 (dd, J=9.0, 2.7 Hz, 1H), 7.79 (d, J=9.0 Hz, 1H), 7.20-7.13 (m, 2H), 7.10-7.04 (m, 2H), 3.88 (s, 3H), 3.55 (s, 1H), 3.43-3.38 (m, 1H), 3.33-3.24 (m, 1H), 3.13-3.06 (m, 1H), 3.01-2.96 (m, 1H), 2.94-2.88 (m, 1H), 2.23-2.09 (m, 1H), 2.06-1.96 (m, 1H). MS (ESI) m/z: 366.1 (M+1)⁺.

### Example 19: Synthesis of (R)-3-(4-methoxyphenyl)-6-nitro-2-(pyrrolidin-3-yl)quinazolin-4 (3H)-one (2l)

Prepared according to Scheme 1.

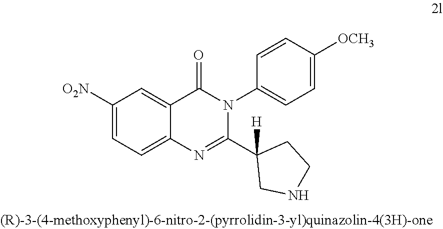 [SMILES: COc1ccc(-n2c([C@@H]3CCNC3)nc3ccc([N+](=O)[O-])cc3c2=O)cc1]

Step 1. Synthesis of tert-butyl (R)-3-(3-(4-methoxyphenyl)-6-nitro-4-oxo-3,4-dihydroquinazolin-2-yl) pyrrolidine-1-carboxylate (1k). 1k (136.20 mg, 58.4 mmol) was prepared from 5-nitroanthranilic acid (91.00 mg, 0.50 mmol), (R)—N-Boc-pyrrolidine-3-carboxylic acid (215.25 mg, 1.00 mmol), triphenyl phosphite (0.40 mL, 1.50 mmol) and 4-methoxyaniline (246.32 mg, 2.00 mmol) in the same manner as described for 1a. Pale yellow solid. ¹H NMR (400 MHz, CDCl₃) δ 9.08 (d, J=2.9 Hz, 1H), 8.53 (dd, J=9.0, 2.6 Hz, 1H), 7.86-7.73 (m, 1H), 7.24-7.13 (m, 2H), 7.12-7.08 (m, 2H), 3.91 (s, 3H), 3.76-3.66 (m, 2H), 3.56-3.55 (m, 1H), 3.31-3.20 (m, 1H), 3.19-3.16 (m, 1H), 2.45-2.30 (m, 1H), 2.08-2.04 (m, 1H), 1.44 (s, 9H). MS (ESI) m/z: 467 (M+1)⁺.

Step 2. Synthesis of (R)-3-(4-methoxyphenyl)-6-nitro-2-(pyrrolidin-3-yl) quinazolin-4 (3H)-one (2l)

Under nitrogen, trifluoroacetic acid (0.16 mL, 2.30 mmol) was added to a solution of 1k (51.85 mg, 0.11 mmol) in dry CH₂Cl₂ (5 mL) at 0° C. Once addition was complete, the ice bath was removed, and the reaction was stirred at rt overnight. Solvent was removed in vacuo, the residue was diluted with CH₂Cl₂ (40 mL) and then washed sequentially with saturated solutions of Na₂CO₃ (1×20 mL) and brine (1×20 mL). The separated organic extract was dried over anhydrous Na₂SO₄, filtered, and then the solvent was evaporated under reduced pressure. The residue was purified by flash chromatography (0% MeOH/CH₂Cl₂˜5% MeOH/CH₂Cl₂) to afford 2l (20.68 mg, 51.3%) as pale yellow solid. ¹H NMR (400 MHz, CDCl₃) δ 9.07 (d, J=2.6 Hz, 1H), 8.51 (dd, J=9.0, 2.7 Hz, 1H), 7.78 (d, J=9.0 Hz, 1H), 7.20-7.13 (m, 2H), 7.10-7.04 (m, 2H), 3.89 (s, 3H), 3.46-3.36 (m, 1H), 3.43-3.38 (m, 1H), 3.33-3.24 (m, 1H), 3.13-3.06 (m, 1H), 3.01-2.96 (m, 1H), 2.94-2.88 (m, 1H), 2.23-2.09 (m, 1H), 2.06-1.96 (m, 1H). MS (ESI) m/z: 366.1 (M+1)⁺.

### Example 20: Synthesis of (R)-4-(6-nitro-4-oxo-2-(pyrrolidin-2-yl)quinazolin-3 (4H)-yl)benzonitrile (2m)

Prepared according to Scheme 1

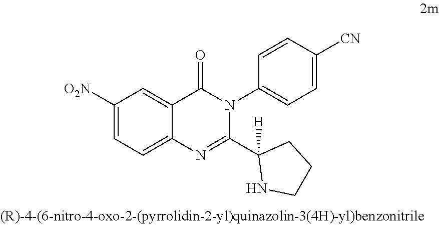 [SMILES: N#Cc1ccc(-n2c([C@H]3CCCN3)nc3ccc([N+](=O)[O-])cc3c2=O)cc1]

Step 1. 5-Nitroanthranilic acid (0.18 g, 1.00 mmol) and N-Boc-D-proline (0.43 g, 2.00 mmol) were dissolved in dry pyridine (6 mL). Triphenyl phosphite (0.80 mL, 3.00 mmol) was added to the mixture and the reaction mixture was heated at 100° C. with MWI for 20 min. 4-Cyanoaniline (0.46 g, 4.00 mmol) was added to the mixture and the reaction mixture was heated at 100° C. with MWI for 1 min. Pyridine was removed in vacuo and the residue was diluted with CH₂Cl₂ (100 mL) and washed with 1N aqueous HCl (50 mL). The organic layer was washed with H₂O (50 mL) and brine (50 mL), dried over anhydrous Na₂SO₄, and filtered, then the solvent was evaporated under reduced pressure. The residue was purified by flash chromatography (0% Ethyl acetate/Hexane to 50% Ethyl acetate/Hexane) to remove most of impurities and pyridine to afford crude product (0.29 g) as pale yellow solid. The crude N—BOC intermediate was taken forward without further purification.

Step 2. Synthesis of (R)-4-(6-nitro-4-oxo-2-(pyrrolidin-2-yl)quinazolin-3 (4H)-yl)benzonitrile (2m)

Under nitrogen, trifluoroacetic acid (0.94 mL, 12.66 mmol) was added to a solution of pale yellow solid described above from step 1 in dry CH₂Cl₂ (18 mL) at 0° C. The solution was stirred at rt overnight, and then the solvent was removed in vacuo. The residue was diluted with CH₂Cl₂ (100 mL), washed sequentially with saturated solutions of Na₂CO₃ (50 mL) and brine (50 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 2m (131.95 mg, 36.5%) as pale yellow solid. ¹H NMR (400 MHz, CDCl₃) δ 9.09 (d, J=2.6 Hz, 1H), 8.58 (dd, J=9.0, 2.6 Hz, 1H), 7.93-7.89 (m, 2H), 7.85 (d, J=9.0 Hz, 1H), 7.53-7.42 (m, 2H), 3.79-3.75 (m, 1H), 3.30-3.22 (m, 1H), 2.81-2.78 (m, 1H), 1.88-1.70 (m, 4H). MS (ESI) m/z: 362.1 (M+1)⁺.

### Example 21: Synthesis of (R)-3-(6-nitro-4-oxo-2-(pyrrolidin-2-yl)quinazolin-3 (4H)-yl)benzonitrile (2n)

Prepared according to Scheme 1.

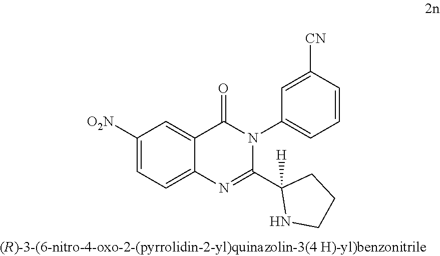 [SMILES: N#Cc1cccc(-n2c([C@H]3CCCN3)nc3ccc([N+](=O)[O-])cc3c2=O)c1]

5-Nitroanthranilic acid (0.18 g, 1.00 mmol) and N-Boc-D-proline (0.43 g, 2.00 mmol) were dissolved in dry pyridine (6 mL). Triphenyl phosphite (0.80 mL, 3.00 mmol) was added to the mixture and the reaction mixture was heated at 100° C. with MWI for 20 min. 3-Cyanoaniline (0.46 g, 4.00 mmol) was added to the mixture and the reaction mixture was heated at 100° C. with MWI for 1 min. Pyridine was removed in vacuo and the residue was diluted with CH₂Cl₂ (100 mL) and washed with 1N HCl aqueous (50 mL). The organic layer was washed with H₂O (50 mL) and brine (50 mL). After separating the organic extract and drying over anhydrous Na₂SO₄, the mixture was filtered, and the solvent was evaporated under reduced pressure. The residue was purified by flash chromatography (0% Ethyl acetate/Hexane˜50% Ethyl acetate/Hexane) to remove most of impurities and pyridine to afford crude product (0.28 g) as pale yellow solid. The crude N—BOC intermediate was taken forward without further purification. Under nitrogen, trifluoroacetic acid (0.89 mL, 12.20 mmol) was added to a solution of pale yellow solid described above in dry CH₂Cl₂ (18 mL) at 0° C. The solution was stirred at rt overnight, and then the solvent was removed in vacuo. The residue was diluted with CH₂Cl₂ (100 mL), washed sequentially with saturated solutions of Na₂CO₃ (50 mL) and brine (50 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 2n (76.66 mg, 21.1%) as pale yellow solid. ¹H NMR (400 MHz, CDCl₃) δ 9.08 (d, J=2.6 Hz, 1H), 8.57 (dd, J=9.0, 2.6 Hz, 1H), 7.89-7.86 (m, 2H), 7.76-7.72 (m, 1H), 7.61-7.53 (m, 2H), 3.81-3.76 (m, 1H), 3.32-3.24 (m, 1H), 2.92-2.76 (m, 1H), 1.88-1.68 (m, 4H). MS (ESI) m/z: 362.1 (M+1)⁺.

### Example 22: Synthesis of (R)-2-(6-nitro-4-oxo-2-(pyrrolidin-2-yl)quinazolin-3 (4H)-yl)benzonitrile (2o)

Prepared according to Scheme 1.

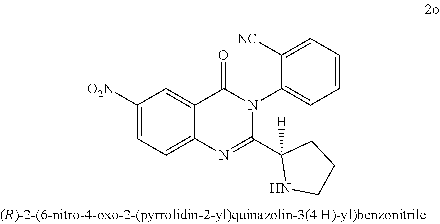 [SMILES: N#Cc1ccccc1-n1c([C@H]2CCCN2)nc2ccc([N+](=O)[O-])cc2c1=O]

### Synthesis of (R)-2-(6-nitro-4-oxo-2-(pyrrolidin-2-yl)quinazolin-3 (4H)-yl)benzonitrile (2o)

5-Nitroanthranilic acid (0.18 g, 1.00 mmol) and N-Boc-D-proline (0.43 g, 2.00 mmol) were dissolved in dry pyridine (6 mL). Triphenyl phosphite (0.80 mL, 3.00 mmol) was added to the mixture and the reaction mixture was heated at 100° C. with MWI for 20 min. 2-Cyanoaniline (0.46 g, 4.00 mmol) was added to the mixture and the reaction mixture was heated at 100° C. with MWI for 1 min. Pyridine was removed in vacuo and the residue was diluted with CH₂Cl₂ (100 mL) and then washed with 1N aqueous HCl (50 mL). The organic layer was subsequently washed with H₂O (50 mL) and then brine (50 mL). The separated organic extract was dried over anhydrous Na₂SO₄. Once filtered, the solvent was evaporated under reduced pressure. The residue was purified by flash chromatography (0% Ethyl acetate/Hexane˜50% Ethyl acetate/Hexane) to remove most of impurities and pyridine to afford crude product (0.18 g) as pale yellow solid. Under nitrogen, trifluoroacetic acid (0.56 mL, 7.80 mmol) was added to a solution of pale yellow solid described above in dry CH₂Cl₂ (18 mL) at 0° C. The solution was stirred at rt overnight, and then the solvent was removed in vacuo. The residue was diluted with CH₂Cl₂ (100 mL), washed sequentially with saturated solutions of Na₂CO₃ (50 mL) and brine (50 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 20 (50.00 mg, 13.8%) as pale yellow solid. ¹H NMR (400 MHz, CDCl₃) δ 9.14 (d, J=2.6 Hz, 1H), 8.60 (dd, J=9.0, 2.6 Hz, 1H), 7.94-7.92 (m, 1H), 7.87-7.83 (m, 2H), 7.77-7.69 (m, 1H), 7.53 (d, J=8.0 Hz, 1H), 3.88-3.77 (m, 1H), 3.30-3.18 (m, 1H), 2.86-2.82 (m, 1H), 1.99-1.61 (m, 4H). MS (ESI) m/z: 362.1 (M+1)⁺.

### Example 23: Synthesis of (R)-3-(4-chlorophenyl)-6-nitro-2-(pyrrolidin-2-yl) quinazolin-4 (3H)-one (2p)

Prepared according to Scheme 1.

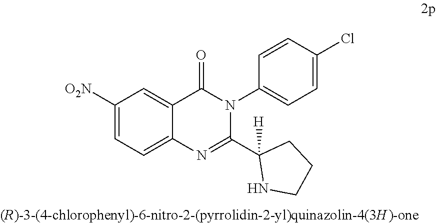 [SMILES: O=c1c2cc([N+](=O)[O-])ccc2nc([C@H]2CCCN2)n1-c1ccc(Cl)cc1]

Step 1. Synthesis of tert-butyl (R)-2-(3-(4-chlorophenyl)-6-nitro-4-oxo-3,4-dihydroquinazolin-2-yl) pyrrolidine-1-carboxylate (11). 11 (86.00 mg) was prepared from 5-nitroanthranilic acid (182.13 mg, 1.00 mmol), (R)—N-Boc-pyrrolidine-3-carboxylic acid (430.00 mg, 2.00 mmol), triphenyl phosphite (0.80 mL, 3.00 mmol) and 4-Chloroaniline (0.51 g, 4.00 mmol) in the same manner as described for 1a. Pale yellow solid. H NMR (500 MHz, CDCl₃) δ 9.09 (dd, J=13.7, 2.6 Hz, 1H), 8.53 (ddd, J=24.1, 9.0, 2.6 Hz, 1H), 7.78 (dd, J=25.1, 9.0 Hz, 1H), 7.49-7.26 (m, 3H), 7.20-7.16 (m, 1H), 4.48-4.45 (m, 1H), 3.83-3.68 (m, 1H), 3.59-3.45 (m, 1H), 2.14-1.80 (m, 4H), 1.42 (s, 5H), 1.28 (s, 4H). MS (ESI) m/z: 471 (M+1)⁺.

Step 2. Synthesis of (R)-3-(4-chlorophenyl)-6-nitro-2-(pyrrolidin-2-yl) quinazolin-4 (3H)-one (2p)

Under nitrogen, trifluoroacetic acid (0.19 mL, 2.60 mmol) was added to a solution of 11 (60.00 mg, 0.13 mmol) in dry CH₂Cl₂ (4.00 mL) at 0° C. The solution was stirred at rt overnight, and then the solvent was removed in vacuo. The residue was diluted with CH₂Cl₂ (10 mL), washed sequentially with saturated solutions of Na₂CO₃ (10 mL) and brine (30 mL), and the separated organic extract was dried over anhydrous Na₂SO₄. Once filtered, the solvent was evaporated under reduced pressure. The residue was purified by flash chromatography (0% MeOH/CH₂Cl₂ to 5% MeOH/CH₂Cl₂) to afford 2p (25.60 mg, 53.1%) as pale yellow solid. ¹H NMR (500 MHz, CDCl₃) δ 9.09 (d, J=2.5 Hz, 1H), 8.55 (dd, J=9.0, 2.6 Hz, 1H), 7.83 (d, J=9.0 Hz, 1H), 7.56 (d, J=8.6 Hz, 2H), 7.29-7.21 (m, 2H), 3.85-3.82 (m, 1H), 3.27-3.25 (m, 1H), 2.83-2.81 (m, 1H), 1.87-1.72 (m, 4H). MS (ESI) m/z: 371.1 (M+1)⁺.

### Example 24: Synthesis of (R)-3-(3-fluoro-4-methoxyphenyl)-6-nitro-2-(pyrrolidin-2-yl) quinazolin-4 (3H)-one (2q)

Prepared according to Scheme 1.

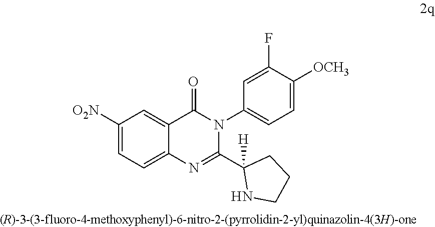 [SMILES: COc1ccc(-n2c([C@H]3CCCN3)nc3ccc([N+](=O)[O-])cc3c2=O)cc1F]

Step 1. Synthesis of tert-butyl (R) 2-(3-(3-fluoro-4-methoxyphenyl)-6-nitro-4-oxo-3,4-dihydroquinazolin-2-yl)pyrrolidine-1-carboxylate (1m). 1m (86.60 mg) was prepared from 5-nitroanthranilic acid (0.18 g, 1.00 mmol), N-Boc-D-proline (0.43 g, 2.00 mmol), triphenyl phosphite (0.80 mL, 3.00 mmol) and 3-fluoro-4-methoxyaniline (0.56 g, 4.00 mmol) in the same manner as described for 1a. Pale yellow solid. ¹H NMR (500 MHz, CDCl₃) δ 9.10 (dd, J=13.9, 2.5 Hz, 1H), 8.52 (ddd, J=23.5, 9.0, 2.5 Hz, 1H), 7.85-7.72 (m, 1H), 7.40-7.08 (m, 2H), 7.04-6.92 (m, 1H), 4.63-4.46 (m, 1H), 3.98 (dd, J=11.7, 3.0 Hz, 3H), 3.81-3.61 (m, 1H), 3.58-3.43 (m, 1H), 2.13-1.83 (m, 4H), 1.42 (d, J=5.3 Hz, 5H), 1.28 (d, J=3.6 Hz, 4H). MS (ESI) m/z: 485 (M+1)⁺.

Step 2. Synthesis of (R)-3-(3-fluoro-4-methoxyphenyl)-6-nitro-2-(pyrrolidin-2-yl) quinazolin-4 (3H)-one (2q)

Under nitrogen, trifluoroacetic acid (0.15 mL, 2.00 mmol) was added to a solution of 1m (50.00 mg, 0.10 mmol) in dry CH₂Cl₂ (3 mL) at 0° C. The solution was stirred at rt overnight, and then the solvent was removed in vacuo. The residue was diluted with CH₂Cl₂ (20 mL), washed sequentially with saturated solutions of Na₂CO₃ (10 mL) and brine (30 mL), and the separated organic extract was dried over anhydrous Na₂SO₄. Once filtered, the solvent was evaporated under reduced pressure. The residue was purified by flash chromatography (0% MeOH/CH₂Cl₂ to 5% MeOH/CH₂Cl₂) to afford 2q (19.80 mg, 51.5%) as pale yellow solid. ¹H NMR (500 MHz, CDCl₃) δ 9.09 (d, J=2.1 Hz, 1H), 8.55 (dd, J=8.9, 2.4 Hz, 1H), 7.82 (d, J=9.0 Hz, 1H), 7.17-6.99 (m, 3H), 3.99 (s, 3H), 3.94-3.83 (m, 1H), 3.33-3.25 (m, 1H), 2.87-2.79 (m, 1H), 1.91-1.70 (m, 4H). MS (ESI) m/z: 385.1 (M+1)⁺.

### Example 25: Synthesis of (R)-3-(2-fluoro-4-methoxyphenyl)-6-nitro-2-(pyrrolidin-2-yl) quinazolin-4 (3H)-one (2r)

Prepared according to Scheme 1.

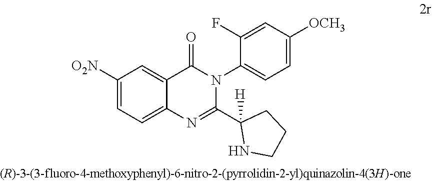 [SMILES: COc1ccc(-n2c([C@H]3CCCN3)nc3ccc([N+](=O)[O-])cc3c2=O)c(F)c1]

Step 1. Synthesis of tert-butyl (R) 2-(3-(2-fluoro-4-methoxyphenyl)-6-nitro-4-oxo-3,4-dihydroquinazolin-2-yl) pyrroli-dine-1-carboxylate (1n). 1n (116.36 mg) was prepared from 5-nitroanthranilic acid (0.18g, 1.00 mmol), N-Boc-D-proline (0.43 g, 2.00 mmol), triphenyl phosphite (0.80 mL, 3.00 mmol) and 2-fluoro-4-methoxyaniline (0.56 g, 4.00 mmol) in the same manner as described for 1a. Pale yellow solid. ¹H NMR (500 MHz, CDCl₃) δ 9.10-9.06 (m, 1H), 8.57-8.45 (m, 1H), 7.78 (dd, J=23.9, 8.9 Hz, 1H), 7.22-7.08 (m, 1H), 6.93-6.81 (m, 2H), 4.60-4.42 (m, 1H), 3.89 (d, J=10.7 Hz, 3H), 3.78-3.47 (m, 2H), 2.10-1.96 (m, 3H), 1.87-1.75 (m, 1H), 1.44 (s, 3H), 1.25 (s, 6H). MS (ESI) m/z: 485 (M+1)⁺.

Step 2. Synthesis of (R)-3-(2-fluoro-4-methoxyphenyl)-6-nitro-2-(pyrrolidin-2-yl) quinazolin-4 (3H)-one (2r)

Under nitrogen, trifluoroacetic acid (0.23 mL, 3.20 mmol) was added to a solution of in (28.00 mg, 0.06 mmol) in dry CH₂Cl₂ (3 mL) at 0° C. The solution was stirred at rt overnight, and then the solvent was removed in vacuo. The residue was diluted with CH₂Cl₂ (30 mL), washed sequentially with saturated solutions of Na₂CO₃ (10 mL) and brine (10 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 2r (14.28 mg, 61.9%) as a pale yellow amorphous solid. ¹H NMR (400 MHz, CDCl₃) δ 9.11 (d, J=2.6 Hz, 1H), 8.56 (dd, J=8.9, 2.6 Hz, 1H), 7.83 (d, J=9.0 Hz, 1H), 7.19 (t, J=8.5 Hz, 1H), 6.91-6.82 (m, 2H), 3.90-3.84 (m, 4H), 3.32-2.28 (m, 1H), 2.91-2.79 (m, 1H), 2.14 (brs, 1H), 1.87-1.70 (m, 4H). MS (ESI) m/z: 385 (M+1)⁺.

### Example 26: Synthesis of (R)-3-(2-methoxy-4-fluorophenyl)-6-nitro-2-(pyrrolidin-2-yl) quinazolin-4 (3H)-one (2s)

Prepared according to Scheme 1.

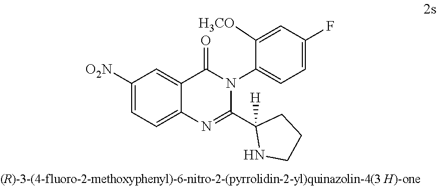 [SMILES: COc1cc(F)ccc1-n1c([C@H]2CCCN2)nc2ccc([N+](=O)[O-])cc2c1=O]

Step 1. Synthesis of tert-butyl (R) 2-(3-(2-methoxy-4-fluorophenyl)-6-nitro-4-oxo-3,4-dihydroquinazolin-2-yl) pyrroli-dine-1-carboxylate (to). 5-Nitroanthranilic acid (182.13 mg, 1.00 mmol) and N-Boc-D-proline (280.00 mg, 1.30 mmol) were dissolved in dry pyridine (5 mL). Triphenyl phosphite (0.80 mL, 3.00 mmol) was added to the mixture and the reaction mixture was heated at 100° C. with MWI for 2×20 min. 2-methoxy-4-fluoroaniline (169.36 mg, 1.20 mmol) was added to the mixture and the reaction mixture was heated at 80° C. with MWI for 2×10 min. Pyridine was removed in vacuo and the residue was diluted with CH₂Cl₂ (60 mL) and washed with 1N aqueous HCl (20 mL). The organic layer was subsequently washed with H₂O (20 mL) and then brine (20 mL). The separated organic extract was dried over anhydrous Na₂SO₄. Once filtered, the solvent was evaporated under reduced pressure. The residue was purified by Waters LC-150 Prep system (5% MeOH/H₂O˜70% MeOH/H₂O) to afford to (60.00 mg, 12.4%) as off-white solid. ¹H NMR (500 MHz, CDCl₃) δ 9.04 (dd, J=11.9, 2.4 Hz, 1H), 8.45 (ddd, J=22.8, 9.0, 2.5 Hz, 1H), 7.71 (dd, J=24.5, 9.0 Hz, 1H), 7.49-7.14 (m, 1H), 6.86-6.66 (m, 2H), 4.60-4.42 (m, 1H), 3.79-3.63 (m, 4H), 3.50-3.30 (m, 1H), 2.09-1.72 (m, 4H), 1.36 (s, 4H), 1.21 (s, 5H). MS (ESI) m/z: 485 (M+1)⁺.

Step 2. Synthesis of (R)-3-(2-methoxy-4-fluorophenyl)-6-nitro-2-(pyrrolidin-2-yl) quinazolin-4 (3H)-one (2s)

Under nitrogen, trifluoroacetic acid (0.15 mL, 2.06 mmol) was added to a solution of to (50.00 mg, 0.10 mmol) in dry CH₂Cl₂ (3 mL) at 0° C. The solution was stirred at rt overnight, and then the solvent was removed in vacuo. The residue was diluted with CH₂Cl₂ (20 mL), washed sequentially with saturated solutions of Na₂CO₃ (10 mL) and brine (10 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 2s (22.60 mg, 58.8%) as a pale yellow amorphous solid. ¹H NMR (500 MHz, CDCl₃) δ 9.11 (d, J=2.5 Hz, 1H), 8.55 (dd, J=8.9, 2.5 Hz, 1H), 7.82 (d, J=9.0 Hz, 1H), 7.25-7.23 (m, 1H), 6.86-6.82 (m, 2H), 3.97-3.90 (m, 1H), 3.80 (s, 3H), 3.28-3.26 (m, 1H), 2.88-2.79 (m, 1H), 1.82-1.71 (m, 3H), 1.61-1.54 (m, 1H). MS (ESI) m/z: 385.1 (M+1)⁺.

### Example 27: Synthesis of 2-(azetidin-2-yl)-6-nitro-3-phenylquinazolin-4 (3H)-one (2t)

Prepared according to Scheme 1.

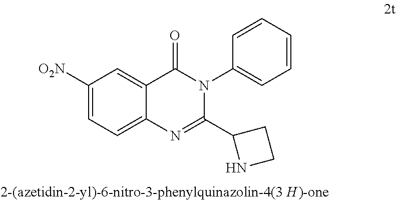 [SMILES: O=c1c2cc([N+](=O)[O-])ccc2nc(C2CCN2)n1-c1ccccc1]

Step 1. Synthesis of 1-(tert-butoxycarbonyl) azetidine-2-carboxylic acid. To a solution of azetidine-2-carboxylic acid (5.00 g, 49.50 mmol), di-tert-butyl dicarbonate (13.50 g, 61.80 mmol) in ethanol (120.0 mL) was added the solution of sodium hydroxide (2.08 g, 51.92 mmol) in H₂O (40.0 mL) at 0° C. After stirring the reaction mixture at rt for 8 hours, the pH of the mixture was adjusted to pH 2 with 1.0N HCl and extracted with ethyl acetate (2×200 mL). The combined organic layers were washed with brine, separated and dried over anhydrous sodium sulfate, then concentrated under reduced pressure to afford 1-(tert-butoxycarbonyl) azetidine-2-carboxylic acid (7.43 g, 74.6%) as off-white solid. ¹H NMR (500 MHz, CDCl₃) δ 4.82-4.78 (m, 1H), 3.92-3.88 (m, 2H), 2.62-2.58 (m, 1H), 2.42-2.38 (m, 1H), 1.45 (s, 9H). MS (ESI) m/z: 202.1 (M+1)⁺.

Step 2. Synthesis of tert-butyl 2-(6-nitro-4-oxo-3-phenyl-3,4-dihydroquinazolin-2-yl) azetidine-1-carboxylate (1p). 5-Nitroanthranilic acid (0.54 g, 3.00 mmol) and 1-(tert-butoxycarbonyl) azetidine-2-carboxylic acid (1.20 g, 6.00 mmol) were dissolved in dry pyridine (12 mL). Triphenyl phosphite (2.36 mL, 9.00 mmol) was added to the mixture and the reaction mixture was heated at 100° C. with MWI for 2×20 min. Aniline (1.12 g, 12.00 mmol) was added to the mixture and the reaction mixture was heated at 100° C. with MWI for 1 min. Pyridine was removed in vacuo and the residue was diluted with CH₂Cl₂ (100 mL) and washed with 1N aqueous HCl (40 mL). The organic layer was washed with H₂O (40 mL) and brine (40 mL), separated and dried over anhydrous Na₂SO₄. After filtration, the solvent was evaporated under reduced pressure. The residue was purified by flash chromatography (0% Ethyl acetate/Hexane˜50% Ethyl acetate/Hexane) to afford 1p (0.59 g, 46.6%) as pale yellow solid. ¹H NMR (500 MHz, CDCl₃) δ 9.13 (d, J=2.4 Hz, 1H), 8.56 (dd, J=8.8, 1.9 Hz, 1H), 7.89 (d, J=9.0 Hz, 1H), 7.66-7.61 (m, 1H), 7.59-7.54 (m, 2H), 7.17-7.08 (m, 2H), 4.79-4.73 (m, 1H), 4.16 (d, J=5.0 Hz, 1H), 3.83 (dd, J=14.1, 8.0 Hz, 1H), 2.24-2.18 (m, 2H), 1.37 (bs, 9H). MS (ESI) m/z: 423 (M+1)⁺.

Step 3. Synthesis of 2-(azetidin-2-yl)-6-nitro-3-phenylquinazolin-4 (3H)-one (2t). Under nitrogen, trifluoroacetic acid (1.75 mL, 23.68 mmol) was added to a solution of 1p (0.50 g, 1.18 mmol) in dry CH₂Cl₂ (16 mL) at 0° C. The solution was stirred at rt overnight, and then the solvent was removed in vacuo. The residue was diluted with CH₂Cl₂ (50 mL), washed sequentially with saturated solutions of Na₂CO₃ (20 mL) and brine (20 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 2t (275.00 mg, 72.2%) as pale yellow solid. ¹H NMR (400 MHz, CDCl₃) δ 9.13 (d, J=2.6 Hz, 1H), 8.58 (dd, J=9.0, 2.7 Hz, 1H), 7.94 (d, J=9.0 Hz, 1H), 7.59-7.55 (m, 3H), 7.28-7.24 (m, 1H), 7.21-7.17 (m, 1H), 4.56 (dd, J=8.4, 6.6 Hz, 1H), 3.58-3.55 (m, 1H), 3.45 (td, J=8.0, 6.8 Hz, 1H), 2.56-2.52 (m, 1H), 2.31-2.22 (m, 1H). MS (ESI) m/z: 323.1 (M+1)⁺.

### Example 28: Synthesis of (R)-5,6-difluoro-3-(4-methoxyphenyl)-2-(pyrrolidin-2-yl) quinazolin-4 (3H)-one (2u)

Prepared according to Scheme 1.

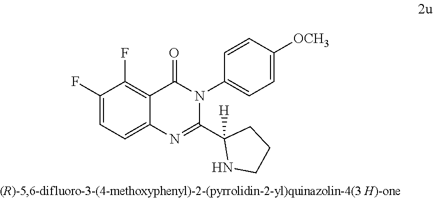 [SMILES: COc1ccc(-n2c([C@H]3CCCN3)nc3ccc(F)c(F)c3c2=O)cc1]

### Synthesis of (R)-5,6-difluoro-3-(4-methoxyphenyl)-2-(pyrrolidin-2-yl) quinazolin-4 (3H)-one (2u)

To a solution of 5,6-difluoroanthranilic acid (0.26 g, 1.50 mmol) in dry pyridine (8 mL) was added N-Boc-D-proline (0.65 g, 3.00 mmol). Triphenyl phosphite (1.18 mL, 4.50 mmol) was added and the reaction mixture was heated at 100° C. with MWI for 20 min. 4-Methoxyaniline (0.73 g, 6.00 mmol) was added to the mixture and the reaction mixture was heated at 100° C. with MWI for 1 min. Pyridine was removed in vacuo and the residue was diluted with CH₂Cl₂ (100 mL) and washed with 1N aqueous HCl (60 mL). The organic layer was subsequently washed with H₂O (60 mL) and then brine (60 mL). The separated organic extract was dried over anhydrous Na₂SO₄. Once filtered, the solvent was evaporated under reduced pressure. The residue was purified by flash chromatography (0% Ethyl acetate/Hexane to 50% Ethyl acetate/Hexane) to remove most of impurities and pyridine to afford crude product (0.65 g) as off-white solid. Material was used without further purification.

Under nitrogen, trifluoroacetic acid (2.08 mL, 28.36 mmol) was added to a solution of off-white solid described above in dry CH₂Cl₂ (20 mL) at 0° C. The solution was stirred at rt overnight, and then the solvent was removed in vacuo. The residue was diluted with CH₂Cl₂ (100 mL), washed sequentially with saturated solutions of Na₂CO₃ (50 mL) and brine (50 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 2u (0.24 g, 44.8%) as a pale yellow amorphous solid. ¹H NMR (400 MHz, CDCl3) δ 7.56 (td, J=9.4, 7.7 Hz, 1H), 7.51-7.45 (m, 1H), 7.22-7.14 (m, 2H), 7.07-7.03 (m, 2H), 3.92-3.86 (m, 4H), 3.29-3.20 (m, 1H), 2.88-2.73 (m, 1H), 1.83-1.56 (m, 4H). MS (ESI) m/z: 358.1 (M+1)⁺.

### Example 29: Synthesis of (S)-5,6-difluoro-3-(4-methoxyphenyl)-2-(1-(tetrahydro-2H-pyran-4-yl) pyrrolidin-2-yl) quinazolin-4 (3H)-one (3h)

Prepared by reductive amination of 2u as shown in Scheme 1.

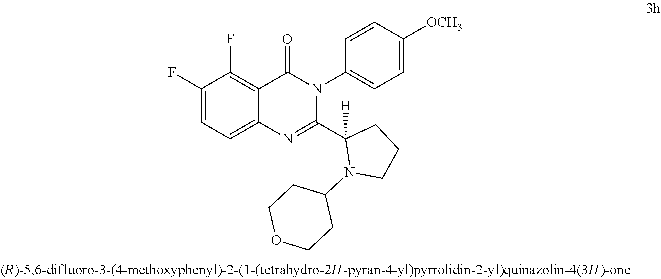 [SMILES: COc1ccc(-n2c([C@H]3CCCN3C3CCOCC3)nc3ccc(F)c(F)c3c2=O)cc1]

### Synthesis of (R)-5,6-difluoro-3-(4-methoxyphenyl)-2-(1-(tetrahydro-2H-pyran-4-yl) pyrrolidin-2-yl) quinazolin-4 (3H)-one (3h)

Acetic acid (0.05 mL) was added to the solution of 2u (53.60 mg, 0.15 mmol) and tetrahydro-4H-pyran-4-one (22.53 mg, 0.23 mmol) in MeOH (2 mL) and CH₂Cl₂ (2 mL), stirred at rt for 1 h, then sodium cyanoborohydride (14.16 mg, 0.23 mmol) was added to the reaction mixture at 0° C., then stirred at room temperature overnight, then the solvent was removed in vacuo and the residue was diluted with CH₂Cl₂ (30 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂-10% MeOH/CH₂Cl₂) to afford 3 h (43.60 mg, 65.9%) as off-white solid. ¹H NMR (400 MHz, CDCl₃) δ 7.68-7.62 (m, 1H), 7.56 (dd, J=17.2, 9.4 Hz, 1H), 7.12-7.06 (m, 4H), 3.97-3.87 (m, 5H), 3.53 (t, J=6.5 Hz, 1H), 3.37-3.24 (m, 3H), 2.71 (s, 1H), 2.59-2.49 (m, 1H), 2.03-1.83 (m, 3H), 1.76-1.72 (m, 1H), 1.67-1.47 (m, 4H). MS (ESI) m/z: 442.1 (M+1)⁺.

### Example 30: Synthesis of (S)-5,6-difluoro-3-(4-methoxyphenyl)-2-(pyrrolidin-2-yl) quinazolin-4 (3H)-one (2v)

Prepared according to Scheme 1.

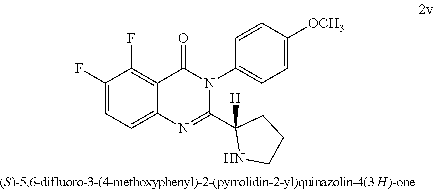 [SMILES: COc1ccc(-n2c([C@@H]3CCCN3)nc3ccc(F)c(F)c3c2=O)cc1]

To a solution of 5,6-difluoroanthranilic acid (0.26 g, 1.50 mmol) in dry pyridine (8 mL) was added N-Boc-L-proline (0.65 g, 3.00 mmol). Triphenyl phosphite (1.18 mL, 4.50 mmol) was added, and the reaction mixture was heated at 100° C. with MWI for 20 min. 4-Methoxyaniline (0.73 g, 6.00 mmol) was added to the mixture and the reaction mixture was heated at 100° C. with MWI for 1 min. Pyridine was removed in vacuo and the residue was diluted with CH₂Cl₂ (100 mL) and washed with 1N aqueous HCl (60 mL). The organic layer was washed with H₂O (60 mL) and brine (60 mL), dried over anhydrous Na₂SO₄, and filtered, then the solvent was evaporated under reduced pressure. The residue was purified by flash chromatography (0% Ethyl acetate/Hexane˜50% Ethyl acetate/Hexane) to remove most of impurities and pyridine to afford crude N—BOC quinazolinone (0.66 g) as off-white solid. Material was carried forward without further purification.

Under nitrogen, trifluoroacetic acid (2.1 mL, 28.4 mmol) was added to a solution of off-white solid described above in dry CH₂Cl₂ (20 mL) at 0° C. The solution was stirred at rt for 12 h, and then the solvent was removed in vacuo. The residue was diluted with CH₂Cl₂ (100 mL), washed sequentially with saturated solutions of Na₂CO₃ (50 mL) and brine (50 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 2v (0.22 g, 41.1%) as a pale yellow amorphous solid. ¹H NMR (400 MHz, CDCl3) δ 7.55 (td, J=9.2, 7.5 Hz, 1H), 7.54-7.46 (m, 1H), 7.25-7.16 (m, 2H), 7.08-7.02 (m, 2H), 3.95-3.86 (m, 4H), 3.25-3.22 (m, 1H), 2.86-2.75 (m, 1H), 1.85-1.56 (m, 4H). MS (ESI) m/z: 358.1 (M+1)⁺.

### Example 31: Synthesis of (S)-5,6-difluoro-3-(4-methoxyphenyl)-2-(1-(tetrahydro-2H-pyran-4-yl) pyrrolidin-2-yl) quinazolin-4 (3H)-one (3i)

Prepared by reductive amination of 2v as shown in Scheme 1.

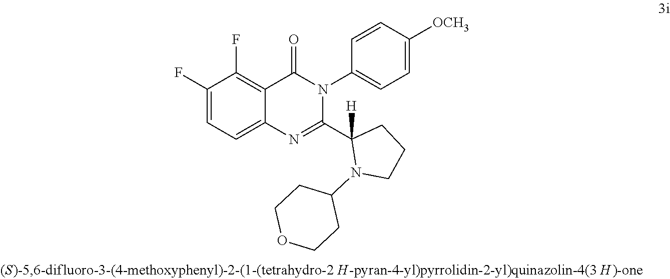 [SMILES: COc1ccc(-n2c([C@@H]3CCCN3C3CCOCC3)nc3ccc(F)c(F)c3c2=O)cc1]

(S)-5,6-difluoro-3-(4-methoxyphenyl)-2-(1-(tetrahydro-2H-pyran-4-yl)pyrrolidin-2-yl)quinazolin-4 (3H)-one Synthesis of (S)-5,6-difluoro-3-(4-methoxyphenyl)-2-(1-(tetrahydro-2H-pyran-4-yl) pyrrolidin-2-yl) quinazolin-4 (3H)-one (3i)

Acetic acid (0.05 mL) was added to the solution of 2v (53.60 mg, 0.15 mmol) and tetrahydro-4H-pyran-4-one (22.53 mg, 0.23 mmol) in MeOH (2 mL) and CH₂Cl₂ (2 mL), stirred at room temperature for 1 h, then sodium cyanoborohydride (14.16 mg, 0.23 mmol) was added to the reaction mixture at 0° C. The solution was stirred at rt overnight, and then the solvent was removed in vacuo. The residue was diluted with CH₂Cl₂ (30 mL), washed sequentially with saturated solutions of Na₂CO₃ (10 mL) and brine (10 mL), and the separated organic extract was dried over anhydrous Na₂SO₄. Once filtered, the solvent was evaporated under reduced pressure. The residue was purified by flash chromatography (0% MeOH/CH₂Cl₂˜10% MeOH/CH₂Cl₂) to afford 3i (36.60 mg, 55.3%) as off-white solid. ¹H NMR (500 MHz, CDCl₃) δ 7.68-7.62 (m, 1H), 7.56 (dd, J=17.3, 9.2 Hz, 1H), 7.12-7.05 (m, 4H), 3.98-3.87 (m, 5H), 3.58-3.50 (m, 1H), 3.39-3.23 (m, 3H), 2.71 (s, 1H), 2.60-2.51 (m, 1H), 2.03-1.86 (m, 3H), 1.78-1.72 (m, 1H), 1.68-1.48 (m, 4H). MS (ESI) m/z: 442.1 (M+1)⁺.

### Example 32: Synthesis of 3-(3-methoxyphenyl)-6-nitro-2-(pyrrolidin-2-yl)quinazolin-4 (3H)-one (2w)

Prepared as shown in Scheme 1.

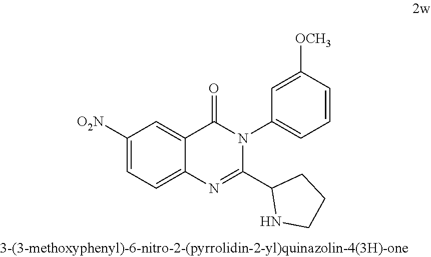 [SMILES: COc1cccc(-n2c(C3CCCN3)nc3ccc([N+](=O)[O-])cc3c2=O)c1]

### Synthesis of 3-(3-methoxyphenyl)-6-nitro-2-(pyrrolidin-2-yl)quinazolin-4 (3H)-one (2w)

To a solution of 5-nitroanthranilic acid (78.6 mg, 0.43 mmol) in dry pyridine (2 mL) was added Boc-DL-proline (180.3 g, 0.84 mmol). After adding triphenyl phosphite (378.5 mg, 1.2 mmol), the reaction mixture was heated at 100° C. with MWI for 20 min. After cooling, 3-methoxyaniline (0.20 mL, 1.8 mmol) was added, and the reaction mixture was heated at 100° C. with MWI for 1 min. Pyridine was removed in vacuo, and the resulting residue was purified by flash chromatography (0-40% EtOAc/Hexanes) to afford N—BOC-quinazolinone (143 mg, 71.5%) as a yellow-orange solid. This N-Boc-intermediate (143 mg, 0.31 mmol) was dissolved in dry CH₂Cl₂ (3 mL) and cooled to 0° C. using an ice-water bath. Trifluoroacetic acid (1.2 mL, 15.6 mmol) was added, and the reaction mixture was allowed to warm to rt. After stirring for 2 h, the reaction mixture was diluted with CH₂Cl₂ (5 mL) and then washed with saturated NaHCO₃ (8 mL). The aqueous phase was extracted with CH₂Cl₂ (15 mL). The combined organic extracts were dried over anhydrous MgSO₄ and then filtered. The solvent was removed in vacuo, and the residue was purified by flash chromatography (0-10% MeOH/CH₂Cl₂) to afford 2w (65.5 mg, 57.7%) as an orange solid. ¹H NMR (400 MHz, CDCl₃) δ 8.90 (dd, J=10.4, 2.6 Hz, 1H), 8.42 (ddd, J=8.9, 2.6, 1.3 Hz, 1H), 7.75 (dd, J=8.9, 4.5 Hz, 1H), 7.57-7.43 (m, 1H), 7.11 (ddt, J=8.5, 3.1, 1.6 Hz, 1H), 7.06-6.98 (m, 1H), 6.85-6.78 (m, 1H), 4.78 (dt, J=23.9, 7.8 Hz, 1H), 3.85 (d, J=13.1 Hz, 3H), 3.66-3.30 (m, 2H), 2.19-1.87 (m, 5H). MS (ESI) m/z: 367.1 (M+1)⁺

### Example 33: Synthesis of (R)-3-(4-methoxyphenyl)-6-nitro-2-(piperidin-2-yl)quinazolin-4 (3H)-one (2x)

Prepared as shown in Scheme 1.

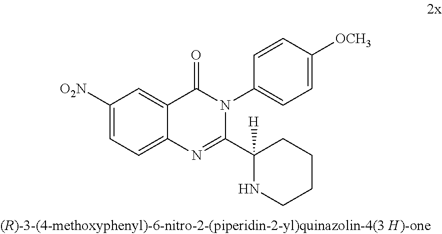 [SMILES: COc1ccc(-n2c([C@H]3CCCCN3)nc3ccc([N+](=O)[O-])cc3c2=O)cc1]

### Synthesis of (R)-3-(4-methoxyphenyl)-6-nitro-2-(piperidin-2-yl)quinazolin-4 (3H)-one (2x)

To a solution of 5-nitroanthranilic acid (103.3 mg, 0.57 mmol) and Fmoc-D-pipecolic acid (260.3 g, 0.74 mmol) in dry pyridine (2 mL) was added triphenyl phosphite (0.45 mL, 1.7 mmol). The reaction mixture was heated at 100° C. with MWI for 1 hr. After cooling, 4-methoxyaniline (61.1 mg, 0.68 mmol) was added, and the reaction mixture was heated at 60° C. with MWI for 30 min. Pyridine was removed in vacuo, and the residue was purified by flash chromatography (0-10% EtOAc/CH₂Cl₂) to afford N-FMOC quinazolinone (87.4 mg, 38.5%) as a yellow solid. The FMOC-intermediate (85 mg, 0.14 mmol) was suspended in dry CH₂Cl₂ (7 mL). Piperidine (0.07 mL, 0.71 mmol) was added to the flask, and the reaction mixture stirred at room temperature for 18 hrs. Upon completion, the reaction mixture was diluted with CH₂Cl₂ (5 mL) and then washed with saturated sodium bicarbonate (8 mL). The organic layer was dried over anhydrous magnesium sulfate and then filtered. The solvent was removed in vacuo. The residue was purified by flash chromatography (0-10% MeOH/CH₂Cl₂) to afford 2× (27.7 mg, 51.6%) as yellow solid. ¹H NMR (400 MHz, CDCl₃) δ 9.10 (d, J=2.6 Hz, 1H), 8.53 (dd, J=9.0, 2.5 Hz, 1H), 7.83 (d, J=9.0 Hz, 1H), 7.28-7.24 (m, 1H), 7.16 (t, J=5.7 Hz, 1H), 7.19-7.04 (m, 3H), 3.90 (s, 3H), 3.47 (d, J=11.05 Hz, 1H), 3.17 (dd, J=13.8, 3.6 Hz, 1H), 2.50 (td, J=13.1, 2.9 Hz, 1H), 2.02 (N—H, 1H), 1.82 (t, J=13.6 Hz, 2H), 1.59-1.31 (m, 3H). MS (ESI) m/z: 381.1 (M+1)⁺

### Example 34. Synthesis of (R)-7-fluoro-3-(4-methoxyphenyl)-4-oxo-2-(pyrrolidin-2-yl)-3,4-dihydroquinazoline-6-carbonitrile (2y)

Prepared as shown in Scheme 1.

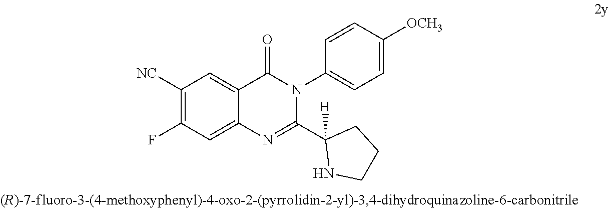 [SMILES: COc1ccc(-n2c([C@H]3CCCN3)nc3cc(F)c(C#N)cc3c2=O)cc1]

### Synthesis of (R)-7-fluoro-3-(4-methoxyphenyl)-4-oxo-2-(pyrrolidin-2-yl)-3,4-dihydroquinazoline-6-carbonitrile (2y)

To a solution of 2-amino-5-cyano-4-fluorobenzoic acid (90.00 mg, 0.50 mmol) in dry pyridine (4 mL) was added N-Boc-D-proline (0.22 g, 1.00 mmol). Triphenyl phosphite (0.40 ml, 1.50 mmol) was added and the reaction mixture was heated at 100° C. with MWI for 20 min. After cooling, 4-methoxyaniline (0.24 g, 2.00 mmol) was added, and the reaction mixture was heated at 100° C. with MWI for 1 min. Pyridine was removed in vacuo, and the residue was diluted with CH₂Cl₂ (80 mL), washed sequentially with 1N aqueous HCl (60 mL), H₂O (40 mL) and brine (40 mL). The separated organic extract was dried over anhydrous Na₂SO₄, filtered, and the solvent was evaporated under reduced pressure. The residue was purified by flash chromatography (0% Ethyl acetate/Hexane˜50% Ethyl acetate/Hexane) to remove most of impurities and pyridine to afford crude product (0.13 g) as pale yellow solid.

Under nitrogen, trifluoroacetic acid (0.86 mL, 11.18 mmol) was added to a solution of pale yellow solid described above in dry CH₂Cl₂ (10 mL) at 0° C. After stirring at rt for 12 h, solvent was removed in vacuo, and the residue was diluted with CH₂Cl₂ (60 mL). The organic layer was washed sequentially with saturated solutions of Na₂CO₃ (30 mL) and brine (30 mL). The separated organic extract was dried over anhydrous Na₂SO₄, filtered, and then the solvent was evaporated under reduced pressure. The residue was purified by flash chromatography (0% MeOH/CH₂Cl₂-10% MeOH/CH₂Cl₂) to afford 2y (18.65 mg, 10.2%) as pale yellow solid. ¹H NMR (500 MHz, CDCl₃) δ 8.57 (d, J=6.9 Hz, 1H), 7.46 (d, J=9.7 Hz, 1H), 7.22-7.18 (m, 1H), 7.17-7.14 (m, 1H), 7.06 (d, J=9.1 Hz, 2H), 3.92-3.85 (m, 4H), 3.28-3.24 (m, 1H), 2.84-2.78 (m, 1H), 1.85-1.67 (m, 4H). MS (ESI) m/z: 365.1 (M+1)⁺.

### Example 35. Synthesis of (R)-6-nitro-2-(pyrrolidin-2-yl)-3-(o-tolyl)quinazolin-4 (3H)-one (2z)

Prepared as shown in Scheme 1.

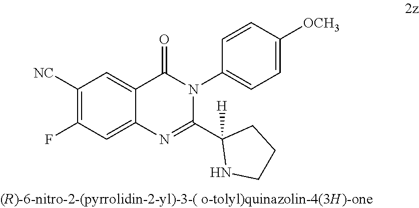 [SMILES: COc1ccc(-n2c([C@H]3CCCN3)nc3cc(F)c(C#N)cc3c2=O)cc1]

### Synthesis of (R)-6-nitro-2-(pyrrolidin-2-yl)-3-(o-tolyl)quinazolin-4 (3H)-one (2z)

To a microwave vial containing 5-nitroanthranilic acid (91.0 mg, 0.5 mmol) and N-Boc-D-proline (0.22 g, 1.0 mmol) was added dry pyridine (5 mL). Triphenyl phosphite (0.4 mL, 1.5 mmol) was added to the vial, and the reaction mixture was heated at 100° C. with MWI for 20 min. After cooling the vial, 2-methylaniline (0.21 mL, 2.0 mmol) was added, and the reaction mixture was heated at 100° C. with MWI for 1 min. Pyridine was removed in vacuo, and the residue was diluted with CH₂Cl₂ (50 mL), and then the organic layer was washed sequentially with 10% citric acid (20 mL) and brine (50 mL). The separated organic extract was dried over MgSO₄, filtered, and concentrated in vacuo under reduced pressure. The crude mixture was purified by flash chromatography (0% ethyl acetate/hexane to 50% ethyl acetate/hexane) to afford N—BOC quinazolinone as a yellow solid. The solid (25.2 mg, 0.056 mmol) was dissolved in dry CH₂Cl₂ (1.5 mL), cooled to 0° C., and trifluoroacetic acid (0.52 mL, 6.86 mmol) was added. After the addition was complete, the reaction mixture was stirred at rt for 8 h. Solvent was removed in vacuo under reduced pressure, and CH₂Cl₂ (10 mL) was added, followed by sequential washing with saturated Na₂CO₃ (5 mL) and brine (5 mL). The separated organic extract was dried over anhydrous MgSO₄, filtered and concentrated in vacuo under reduced pressure. The crude mixture was purified by flash chromatography (0% MeOH/CH₂Cl₂˜5% MeOH/CH₂Cl₂) to afford 2z (12.3 mg, 63%) as a brown-reddish solid. ¹H NMR (500 MHz, chloroform-d δ 9.07 (d, J=2.6 Hz, 1H), 8.49 (dd, J=9.0, 2.7 Hz, 1H), 7.77 (d, J=9.0 Hz, 1H), 7.48-7.27 (m, 3H), 7.10 (dd, J=7.6, 1.3 Hz, 1H), 3.53 (t, J=7.0 Hz, 1H), 3.23 (ddd, J=11.4, 6.8, 4.8 Hz, 1H), 2.72 (dt, J=10.9, 6.6 Hz, 1H), 2.06 (s, 3H), 1.82-1.57 (m, 4H). MS (ESI) m/z: 351.1 (M+1)⁺.

### Example 36. Synthesis of (R)-6-nitro-2-(pyrrolidin-2-yl)-3-(m-tolyl)quinazolin-4 (3H)-one (2aa)

Prepared as shown in Scheme 1.

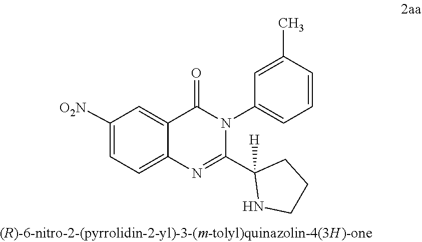 [SMILES: Cc1cccc(-n2c([C@H]3CCCN3)nc3ccc([N+](=O)[O-])cc3c2=O)c1]

### Synthesis of (R)-6-nitro-2-(pyrrolidin-2-yl)-3-(m-tolyl)quinazolin-4 (3H)-one (2aa)

To a microwave vial containing 5-nitroanthranilic acid (91.0 mg, 0.5 mmol) and N-Boc-D-proline (0.22 g, 1.0 mmol) was added dry pyridine (5 mL). Triphenyl phosphite (0.4 mL, 1.5 mmol) was added to the vial, and the reaction mixture was heated at 100° C. with MWI for 20 min. After cooling the vial, 3-methylaniline (0.21 mL, 2.0 mmol) was added, and the reaction mixture was heated at 100° C. with MWI for 1 min. Pyridine was removed in vacuo, and the residue was diluted with CH₂Cl₂ (50 mL), and then the organic layer was washed sequentially with 10% citric acid (20 mL) and brine (50 mL). The separated organic extract was dried over MgSO₄, filtered, and concentrated in vacuo under reduced pressure. The crude mixture was purified by flash chromatography (0% Ethyl acetate/hexane˜50% ethyl acetate/hexane) to afford N—BOC quinazolinone as a yellow solid. The solid (23.1 mg, 0.052 mmol) was dissolved in dry CH₂Cl₂ (1.5 mL), cooled to 0° C., and trifluoroacetic acid (0.48 mL, 6.37 mmol) was added. After the addition was complete, the reaction mixture was stirred at rt for 8 h. Solvent was removed in vacuo under reduced pressure, and CH₂Cl₂ (10 mL) was added, followed by sequential washing with saturated Na₂CO₃ (5 mL) and brine (5 mL). The separated organic extract was dried over anhydrous MgSO₄, filtered and concentrated in vacuo under reduced pressure. The crude mixture was purified by flash chromatography (0% MeOH/CH₂Cl₂˜5% MeOH/CH₂Cl₂) to afford 2aa (4.8 mg, 26%) as a brown-reddish amorphous solid. ¹H NMR (500 MHz, Chloroform-d) δ 9.05 (d, J=2.6 Hz, 1H), 8.48 (dd, J=9.0, 2.6 Hz, 1H), 7.75 (d, J=9.0 Hz, 1H), 7.39 (t, J=7.8 Hz, 1H), 7.29 (d, J=7.8 Hz, 1H), 7.04 (d, J=7.9 Hz, 1H), 6.99 (d, J=6.7 Hz, 1H), 3.85 (td, J=7.2, 3.1 Hz, 1H), 3.28-3.14 (m, 1H), 2.77 (dt, J=12.9, 6.6 Hz, 1H), 2.37 (d, J=10.9 Hz, 3H), 1.81-1.60 (m, 4H). MS (ESI) m/z: 351.1 (M+1)⁺.

### Example 37. Synthesis of (R)-6-nitro-2-(pyrrolidin-2-yl)-3-(p-tolyl)quinazolin-4 (3H)-one (2bb)

Prepared as shown in Scheme 1.

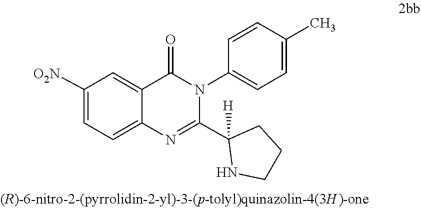 [SMILES: Cc1ccc(-n2c([C@H]3CCCN3)nc3ccc([N+](=O)[O-])cc3c2=O)cc1]

### Synthesis of (R)-6-nitro-2-(pyrrolidin-2-yl)-3-(p-tolyl)quinazolin-4 (3H)-one (2bb)

To a microwave vial containing 5-nitroanthranilic acid (91.0 mg, 0.5 mmol) and N-Boc-D-proline (0.22 g, 1.0 mmol) was added dry pyridine (5 mL). Triphenyl phosphite (0.4 mL, 1.5 mmol) was added to the vial, and the reaction mixture was heated at 100° C. with MWI for 20 min. After cooling the vial, 4-methylaniline (0.21 mL, 2.0 mmol) was added, and the reaction mixture was heated at 100° C. with MWI for 1 min. Pyridine was removed in vacuo, and the residue was diluted with CH₂Cl₂ (50 mL), and then the organic layer was washed sequentially with 10% citric acid (20 mL) and brine (50 mL). The separated organic extract was dried over MgSO₄, filtered, and concentrated in vacuo under reduced pressure. The crude mixture was purified by flash chromatography (0% Ethyl acetate/Hexane˜50% Ethyl acetate/Hexane) to afford N—BOC-quinazolinone as a yellow solid. The solid (22.4 mg, 0.050 mmol) was dissolved in dry CH₂Cl₂ (1.5 mL), cooled to 0° C., and trifluoroacetic acid (0.47 mL, 6.13 mmol) was added. After the addition was complete, the reaction mixture was stirred at rt for 8 h. Solvent was removed in vacuo under reduced pressure, and CH₂Cl₂ (10 mL) was added, followed by sequential washing with saturated Na₂CO₃ (5 mL) and brine (5 mL). The separated organic extract was dried over anhydrous MgSO₄, filtered and concentrated in vacuo under reduced pressure. The crude mixture was purified by flash chromatography (0% MeOH/CH₂Cl₂ to 5% MeOH/CH₂Cl₂) to afford 2bb (4.9 mg, 28%) as a brown-reddish oil. ¹H NMR (500 MHz, Chloroform-d) δ 9.05 (d, J=2.6 Hz, 1H), 8.48 (dd, J=9.0, 2.6 Hz, 1H), 7.75 (d, J=8.9 Hz, 1H), 7.31 (d, J=8.1 Hz, 2H), 7.09 (ddd, J=22.4, 8.9, 2.2 Hz, 2H), 3.84 (t, J=7.3 Hz, 1H), 3.21 (dt, J=10.6, 5.2 Hz, 1H), 2.75 (dd, J=11.2, 6.9 Hz, 1H), 2.40 (s, 3H), 1.81-1.58 (m, 4H). MS (ESI) m/z: 351.1 (M+1)⁺.

### Example 38. Synthesis of 3-(2-chlorophenyl)-6-nitro-2-(pyrrolidin-2-yl) quinazolin-4 (3H)-one (2cc). (2cc)

Prepared as shown in Scheme 1.

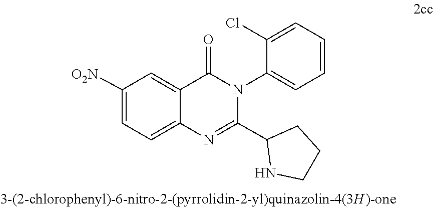 [SMILES: O=c1c2cc([N+](=O)[O-])ccc2nc(C2CCCN2)n1-c1ccccc1Cl]

### Synthesis of 3-(2-chlorophenyl)-6-nitro-2-(pyrrolidin-2-yl) quinazolin-4 (3H)-one (2cc)

To a microwave vial containing 5-nitroanthranilic acid (91.0 mg, 0.5 mmol) and N-Boc-DL-proline (0.22 g, 1.0 mmol) was added dry pyridine (5 mL). Triphenyl phosphite (0.4 mL, 1.5 mmol) was added to the vial, and the reaction mixture was heated at 100° C. with MWI for 20 min. After cooling the vial, 2-chloroaniline (0.21 mL, 2.0 mmol) was added, and the reaction mixture was heated at 100° C. with MWI for 1 min. Pyridine was removed in vacuo, and the residue was diluted with CH₂Cl₂ (50 mL), and then the organic layer was washed sequentially with 10% citric acid (20 mL) and brine (50 mL). The separated organic extract was dried over MgSO₄, filtered, and concentrated in vacuo under reduced pressure. The crude mixture was purified by flash chromatography (0% ethyl acetate/hexane to 50% ethyl acetate/hexane) to afford the N—BOC quinazolinone as a yellow solid. The solid (14.6 mg, 0.031 mmol) was dissolved in dry CH₂Cl₂ (1.5 mL), cooled to 0° C., and trifluoroacetic acid (0.05 mL, 0.62 mmol) was added. After the addition was complete, the reaction mixture was stirred at rt for 8 h. Solvent was removed in vacuo under reduced pressure, and CH₂Cl₂ (10 mL) was added, followed by sequential washing with saturated Na₂CO₃ (5 mL) and brine (5 mL). The separated organic extract was dried over anhydrous MgSO₄, filtered and concentrated in vacuo under reduced pressure. The crude mixture was purified by flash chromatography (0% MeOH/CH₂Cl₂˜5% MeOH/CH₂Cl₂) to afford 2cc (3.0 mg, 26%) as a brown-reddish oil. ¹H NMR (500 MHz, Chloroform-d) δ 9.08 (d, J=2.6 Hz, 1H), 8.51 (dd, J=9.0, 2.7 Hz, 1H), 7.78 (d, J=9.0 Hz, 1H), 7.58 (dd, J=7.8, 1.7 Hz, 1H), 7.45 (dtd, J=16.8, 7.5, 1.7 Hz, 3H), 7.36 (dd, J=7.5, 1.9 Hz, 1H), 3.81 (t, J=7.4 Hz, 1H), 3.22-3.12 (m, 1H), 2.77 (dt, J=10.8, 6.6 Hz, 1H), 1.88-1.76 (m, 2H), 1.71-1.55 (m, 2H). MS (ESI) m/z: 371.1 (M+1)⁺.

### Example 39. Synthesis of 3-(3-chlorophenyl)-6-nitro-2-(pyrrolidin-2-yl) quinazolin-4 (3H)-one (2dd)

Prepared as shown in Scheme 1.

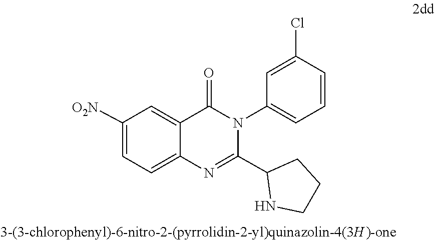 [SMILES: O=c1c2cc([N+](=O)[O-])ccc2nc(C2CCCN2)n1-c1cccc(Cl)c1]

### Synthesis of 3-(3-chlorophenyl)-6-nitro-2-(pyrrolidin-2-yl) quinazolin-4 (3H)-one (2dd)

To a microwave vial containing 5-nitroanthranilic acid (91.0 mg, 0.5 mmol) and N-Boc-D-proline (0.22 g, 1.0 mmol) was added dry pyridine (5 mL). Triphenyl phosphite (0.4 mL, 1.5 mmol) was added to the vial, and the reaction mixture was heated at 100° C. with MWI for 20 min. After cooling the vial, 3-chloroaniline (0.21 mL, 2.0 mmol) was added, and the reaction mixture was heated at 100° C. with MWI for 1 min. Pyridine was removed in vacuo, and the residue was diluted with CH₂Cl₂ (50 mL), and then the organic layer was washed sequentially with 10% citric acid (20 mL) and brine (50 mL). The separated organic extract was dried over MgSO₄, filtered, and concentrated in vacuo under reduced pressure. The crude mixture was purified by flash chromatography (0% Ethyl acetate/Hexane˜50% Ethyl acetate/Hexane) to afford N—BOC-quinazolinone as a yellow solid. The solid (34.9 mg, 0.074 mmol) was dissolved in dry CH₂Cl₂ (1.5 mL), cooled to 0° C., and trifluoroacetic acid (0.11 mL, 0.48 mmol) was added. After the addition was complete, the reaction mixture was stirred at rt for 8 h. Solvent was removed in vacuo under reduced pressure, and CH₂Cl₂ (10 mL) was added, followed by sequential washing with saturated Na₂CO₃ (5 mL) and brine (5 mL). The separated organic extract was dried over anhydrous MgSO₄, filtered and concentrated in vacuo under reduced pressure. The crude mixture was purified by flash chromatography (0% MeOH/CH₂Cl₂˜5% MeOH/CH₂Cl₂) to afford 2dd (4.1 mg, 15%) as a brown-reddish oil. ¹H NMR (500 MHz, Chloroform-d) δ 9.03 (d, J=2.6 Hz, 1H), 8.49 (dd, J=8.9, 2.7 Hz, 1H), 7.76 (d, J=9.0 Hz, 1H), 7.51-7.41 (m, 3H), 7.28 (t, J=2.0 Hz, 1H), 7.23 (t, J=2.0 Hz, 1H), 7.16 (dt, J=7.1, 1.8 Hz, 1H), 7.12 (dt, J=6.6, 2.1 Hz, 1H), 3.77 (dt, J=11.4, 7.1 Hz, 1H), 3.26-3.14 (m, 1H), 2.75 (dq, J=10.9, 6.9 Hz, 1H), 1.84-1.60 (m, 5H). MS (ESI) m/z: 371.1 (M+1)⁺.

### Example 40. Synthesis of 3-(3-fluorophenyl)-6-nitro-2-(pyrrolidin-2-yl)quinazolin-4 (3H)-one (2ee)

Prepared as shown in Scheme 1.

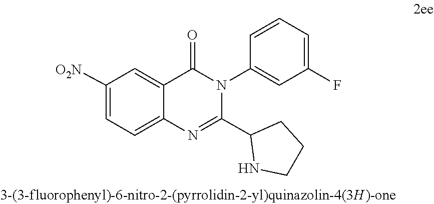 [SMILES: O=c1c2cc([N+](=O)[O-])ccc2nc(C2CCCN2)n1-c1cccc(F)c1]

### Synthesis of 3-(3-fluorophenyl)-6-nitro-2-(pyrrolidin-2-yl)quinazolin-4 (3H)-one (2ee)

To a solution of 5-nitroanthranilic acid (79.0 mg, 0.43 mmol) in dry pyridine (2 mL) was added Boc-DL-proline (158.0 g, 0.73 mmol). After adding triphenyl phosphite (0.3 mL, 1.3 mmol), the reaction mixture was heated at 100° C. with MWI for 20 min. After cooling, 3-fluoroaniline (0.16 mL, 1.7 mmol) was added, and the reaction mixture was heated at 100° C. with MWI for 2 min. Pyridine was removed in vacuo and the resulting residue was purified by flash chromatography (0-5% EtOAc/CH₂Cl₂) to crudely afford the N—BOC quinazolinone intermediate (141.1 mg)

Under nitrogen, this N-Boc-intermediate (141.1 mg, 0.31 mmol) was dissolved in dry CH₂Cl₂ (4.5 mL). Trifluoroacetic acid (2.9 mL) was added dropwise. After stirring for 1 h, the reaction mixture was quenched with saturated NaHCO₃ (10 mL). The organic layer was collected and then the remaining aqueous layer was extracted with CH₂Cl₂ (15 mL). The organic extracts were combined and dried over MgSO₄. The solvent was removed in vacuo, and the residue was purified by flash chromatography (0-3% MeOH/CH₂Cl₂) to afford 3-(3-fluorophenyl)-6-nitro-2-(pyrrolidin-2-yl)quinazolin-4 (3H)-one (2ee) (30.7 mg, 28%) as a brown solid. ¹H NMR (500 MHz, CDCl₃) δ 8.96 (d, J=2.6 Hz, 1H), 8.48 (dt, J=9.0, 2.6 Hz, 1H), 7.76 (d, J=8.9 Hz, 1H), 7.55 (dtd, J=28.0, 8.2, 5.9 Hz, 1H), 7.30-7.20 (m, −2H), 7.05-6.95 (m, 1H), 4.67 (dt, J=39.1, 7.8 Hz, 1H), 3.56-3.29 (m, 4H), 2.12-1.84 (m, 4H). MS (ESI) m/z: 355.0 (M+1)⁺

### Example 41. Synthesis of 3-(2-fluorophenyl)-6-nitro-2-(pyrrolidin-2-yl)quinazolin-4 (3H)-one (2ff)

Prepared as shown in Scheme 1.

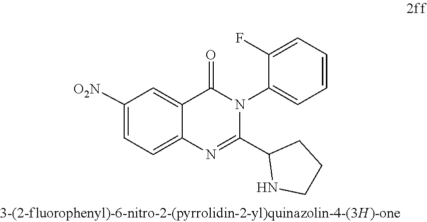 [SMILES: O=c1c2cc([N+](=O)[O-])ccc2nc(C2CCCN2)n1-c1ccccc1F]

### Synthesis of 3-(2-fluorophenyl)-6-nitro-2-(pyrrolidin-2-yl)quinazolin-4 (3H)-one (2ff)

To a solution of 5-nitroanthranilic acid (152.1 mg, 0.84 mmol) in dry pyridine (4 mL) was added N-Boc-DL-proline (354.0 mg, 1.6 mmol). After adding triphenyl phosphite (0.6 mL, 2.6 mmol), the reaction mixture was heated at 100° C. with MWI for 20 min. After cooling, 2-fluoroaniline (0.32 mL, 3.3 mmol) was added, and the reaction mixture was heated at 100° C. with MWI for 1 min. Pyridine was removed in vacuo and the resulting residue was purified by flash chromatography (25% EtOAc/Hexanes) to afford the N—BOC quinazolinone intermediate (64.2 mg, 17%) as a solid. This N-Boc-intermediate (64.2 mg, 0.17 mmol) was dissolved in dry CH₂Cl₂ (3 mL). Trifluoroacetic acid (0.3 mL) was added dropwise. After stirring for 2 h, the reaction mixture was quenched with saturated NaHCO₃ (10 mL). The aqueous phase was extracted with CH₂Cl₂ (15 mL). The organic extracts were combined and dried over anhydrous MgSO₄ and then filtered. The solvent was removed in vacuo, and the residue was purified by flash chromatography (0-3% MeOH/CH₂Cl₂) to afford 3-(2-fluorophenyl)-6-nitro-2-(pyrrolidin-2-yl)quinazolin-4 (3H)-one (2ff) (35.6 mg, 55.5%) as a yellow solid. ¹H NMR (500 MHz, CDCl₃) δ 9.13 (d, J=2.6 Hz, 1H), 8.58 (dd, J=9.0, 2.7 Hz, 1H), 7.85 (d, J=8.9 Hz, 1H), 7.61-7.54 (m, 1H), 7.41-7.31 (m, 3H), 3.95 (dd, J=8.1, 5.9 Hz, 1H), 3.25 (dt, J=10.6, 6.1 Hz, 1H), 2.81 (dt, J=10.8, 6.5 Hz, 1H), 1.86-1.52 (m, 4H). MS (ESI) m/z: 355.0 (M+1)⁺

### Example 42. Synthesis of 3-(4-fluorophenyl)-6-nitro-2-(pyrrolidin-2-yl)quinazolin-4 (3H)-one (2gg)

Prepared as shown in Scheme 1.

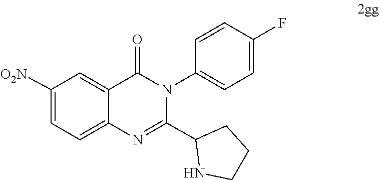 [SMILES: O=c1c2cc([N+](=O)[O-])ccc2nc(C2CCCN2)n1-c1ccc(F)cc1]

### Synthesis of 3-(4-fluorophenyl)-6-nitro-2-(pyrrolidin-2-yl)quinazolin-4 (3H)-one (2gg)

To a solution of 5-nitroanthranilic acid (105.1 mg, 0.58 mmol) in dry pyridine (2 mL) was added N-Boc-DL-proline (154.9 g, 0.72 mmol). After adding triphenyl phosphite (0.45 mL, 1.7 mmol), the reaction mixture was heated at 100° C. with MWI for 40 min. After cooling, 4-fluoroaniline (0.09 mL, 0.68 mmol) was added, and the reaction mixture was heated at 60° C. with MWI for 3 min. The reaction mixture was diluted in CH₂Cl₂ and washed with 10% citric acid. The aqueous phase was washed with CH₂Cl₂ (20 mL) and dried over anhydrous MgSO₄ and the resulting residue was purified by flash chromatography (25% EtOAc/Hexanes) to afford the N—BOC quinazolinone intermediate (66.9 mg, 25%) as a solid. Under nitrogen, this N-Boc-intermediate (66.9 mg, 0.15 mmol) was dissolved in anhydrous 1,4-dioxane (0.20 mL) and cooled to 0° C. using an ice-water bath. To this cooled solution, 4N HCl in dioxane (0.20 mL, 0.74 mmol) was added dropwise, and the reaction mixture was allowed to warm to rt. After stirring for 23 h, solvent was removed in vacuo. The residue was diluted with EtOAc (5 mL) and then washed with saturated Na₂CO₃ (8 mL). The layers were separated, and the aqueous phase was extracted with EtOAc (15 mL). The combined organic extracts were dried over anhydrous MgSO₄ and then filtered. The solvent was removed in vacuo, and the residue was purified by flash chromatography (5% MeOH/CH₂Cl₂) to afford 3-(4-fluorophenyl)-6-nitro-2-(pyrrolidin-2-yl)quinazolin-4 (3H)-one (2gg) (28.3 mg, 53%) as an orange solid. ¹H NMR (500 MHz, CDCl₃) δ 9.11 (d, J=2.6 Hz, 1H), 8.56 (dd, J=9.0, 2.6 Hz, 1H), 7.83 (d, J=9.0 Hz, 1H), 7.30 (m, 4H), 3.85 (t, J=7.2 Hz, 1H), 3.27 (ddd, J=11.5, 8.0, 4.7 Hz, 1H), 2.81 (dt, J=10.8, 6.8 Hz, 1H), 1.79 (m, 4H). MS (ESI) m/z: 355.0 (M+1)⁺

### Example 43. Synthesis of (R)-3-(4-methoxyphenyl)-7-methyl-6-nitro-2-(pyrrolidin-2-yl) quinazolin-4 (3H)-one (2hh)

Prepared as shown in Scheme 1.

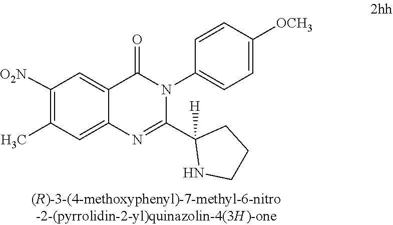 [SMILES: COc1ccc(-n2c([C@H]3CCCN3)nc3cc(C)c([N+](=O)[O-])cc3c2=O)cc1]

### Synthesis of (R)-3-(4-methoxyphenyl)-7-methyl-6-nitro-2-(pyrrolidin-2-yl) quinazolin-4 (3H)-one (2hh)

To a 25 mL round bottom flask, 2-amino-4-amino benzoic acid (1.51g, 10 mmmol) and acetic anhydride (2.9 mL, 30 mmol) were charged and heated at 137° C. for 2 hrs. Then, the reaction mixture was poured into crushed ice and stirred for 12 hrs. Finally, the reaction mixture was worked-up by suction filtration, followed by a wash with methanol and drying over magnesium sulfate to afford 2-acetylamido-4-methyl-5-nitrobenzoic acid. 2-acetylamido-4-methyl-5-nitrobenzoic acid (3.37g, 17.4 mmol) was dissolved in 42.3 mL of sulfuric acid and the reaction mixture was lowered to 0° C. Then, using addition funnel, 4.92 mL of fuming nitric acid was added dropwise and the reaction mixture is stirred for 12 hrs in ice bath. Finally, the reaction mixture is worked up by pouring it over crushed ice, filtering, and vacuum drying it overnight to afford 5-nitro-4-methylanthranilic acid. To a microwave vial containing 5-nitro-3-methylanthranilic acid (98.0 g, 0.5 mmol) and N-Boc-D-proline (0.140 g, 0.65 mmol) was added dry pyridine (1.5 mL). Triphenyl phosphite (0.4 mL, 1.5 mmol) was added to the vial, and the reaction mixture was heated at 100° C. with MWI for 60 min. After cooling the vial, 4-methoxyaniline (74 mg, 1.2 mmol) was added, and the reaction mixture was heated at 60° C. with MWI for 30 min. Pyridine was removed in vacuo, and the residue was diluted with CH₂Cl₂ (50 mL), and then the organic layer was washed sequentially with 10% citric acid (20 mL) and brine (50 mL). The separated organic extract was dried over MgSO₄, filtered, and concentrated in vacuo under reduced pressure. The crude mixture was purified by flash chromatography (3% Ethyl acetate/DCM) to afford the N-Boc intermediate as a yellow solid. The solid (0.119 g, 0.248 mmol) was dissolved in dry dioxane (1.5 mL), cooled to 0° C., and 4 M HCl/dioxane (0.31 mL, 1.24 mmol) was added. After the addition was complete, the reaction mixture was stirred at rt for 8 h. Solvent was removed in vacuo under reduced pressure, and CH₂Cl₂ (10 mL) was added, followed by sequential washing with saturated Na₂CO₃ (5 mL) and brine (5 mL). The separated organic extract was dried over anhydrous MgSO₄, filtered and concentrated in vacuo under reduced pressure. The crude mixture was purified by flash chromatography (0% MeOH/CH₂Cl₂˜10% MeOH/CH₂Cl₂) to afford 2hh (59 mg, 62%) as a yellow solid. ¹H NMR (400 MHz, Chloroform-d) δ 8.83 (s, 1H), 7.63 (s, 1H), 7.24-7.12 (m, 2H), 7.10-7.01 (m, 2H), 3.88 (s, 3H), 3.25 (ddd, J=11.0, 7.7, 3.7 Hz, 1H), 2.91-2.75 (m, 3H), 2.73 (s, 3H), 1.75 (qdd, J=12.3, 6.6, 3.4 Hz, 4H). MS (ESI) m/z: 381.2 (M+1)⁺.

### Example 44. Synthesis of (R)-6-nitro-2-(pyrrolidin-2-yl)-3-(4-(trifluoromethoxy)phenyl)quinazolin-4 (3H)-one (2ii)

Prepared as shown in Scheme 1

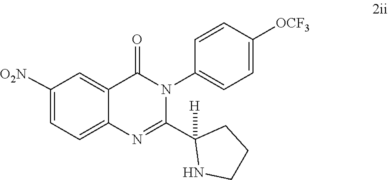 [SMILES: O=c1c2cc([N+](=O)[O-])ccc2nc([C@H]2CCCN2)n1-c1ccc(OC(F)(F)F)cc1]

### Synthesis of (R)-6-nitro-2-(pyrrolidin-2-yl)-3-(4-(trifluoromethoxy)phenyl)quinazolin-4 (3H)-one (2ii)

To a solution of 5-nitroanthranilic acid (102.8 mg, 0.56 mmol) in dry pyridine (2 mL) was added N-Boc-D-proline (158.0 g, 0.73 mmol). After adding triphenyl phosphite (0.30 mL, 1.3 mmol), the reaction mixture was heated at 100° C. with MWI for 40 min. After cooling, 4-trifluoromethoxyaniline (0.09 mL, 0.68 mmol) was added, and the reaction mixture was heated at 60° C. with MWI for 15 min. The reaction mixture was diluted with CH₂Cl₂ and washed with 10% citric acid (15 mL). The layers were separated and the aqueous phase was washed with CH₂Cl₂ (30 mL). The organic extracts were combined, dried over anhydrous MgSO₄, and then filtered. Solvent was removed in vacuo and the resulting residue was purified by flash chromatography (5% EtOAc/CH₂Cl₂) to afford the N—BOC quinazolinone intermediate (66.8 mg, 22.9%)

Under nitrogen, this N-Boc-intermediate (66.8 mg, 0.13 mmol) was dissolved in anhydrous 1,4-dioxane (0.20 mL) and cooled to 0° C. using an ice-water bath. To this cooled solution, 4N HCl in dioxane (0.16 mL, 0.64 mmol) was added dropwise, and the reaction mixture was allowed to warm to rt. After stirring for 15 h, the reaction mixture was diluted with CH₂Cl₂ (5 mL) and then washed with saturated NaHCO₃ (8 mL). The layers were separated, and the aqueous phase was extracted with CH₂Cl₂ (15 mL). The organic extracts were combined, dried over anhydrous MgSO₄, and then filtered. The solvent was removed in vacuo, and the residue was purified by flash chromatography (2% MeOH/CH₂Cl₂) to afford (R)-6-nitro-2-(pyrrolidin-2-yl)-3-(4-(trifluoromethoxy)phenyl)quinazolin-4 (3H)-one 2ii (31.5 mg, 57.6%) as an orange solid. ¹H NMR (500 MHz, CDCl₃) δ 9.10 (d, J=2.6 Hz, 1H), 8.56 (dd, J=9.0, 2.7 Hz, 1H), 7.83 (d, J=8.9 Hz, 1H), 7.43 (d, J=8.6 Hz, 2H), 7.37 (dd, J=9.1, 2.5 Hz, 1H), 7.34-7.30 (m, 1H), 3.83 (t, J=6.9 Hz, 1H), 3.27 (ddd, J=10.8, 7.7, 4.1 Hz, 1H), 2.81 (dt, J=12.6, 6.2 Hz, 1H), 1.91-1.67 (m, 4H). MS (ESI) m/z: 420.9 (M+1)⁺

### Example 45. Synthesis of (R)-6-nitro-2-(pyrrolidin-2-yl)-3-(3-(trifluoromethoxy)phenyl)quinazolin-4 (3H)-one (2jj)

Prepared as shown in Scheme 1

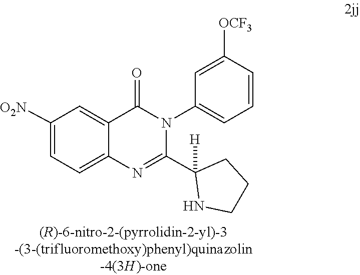 [SMILES: O=c1c2cc([N+](=O)[O-])ccc2nc([C@H]2CCCN2)n1-c1cccc(OC(F)(F)F)c1]

### Synthesis of (R)-6-nitro-2-(pyrrolidin-2-yl)-3-(3-(trifluoromethoxy)phenyl)quinazolin-4 (3H)-one (2jj)

To a solution of 5-nitroanthranilic acid (102.8 mg, 0.56 mmol) in dry pyridine (2 mL) was added Boc-D-proline (160.0 g, 0.74 mmol). After adding triphenyl phosphite (0.45 mL, 1.7 mmol), the reaction mixture was heated at 100° C. with MWI for 40 min. After cooling, 3-trifluoromethoxyaniline (0.09 mL, 0.68 mmol) was added, and the reaction mixture was heated at 60° C. with MWI for 15 min. The reaction mixture was diluted in CH₂Cl₂ and washed with 10% citric acid. The aqueous phase was washed with CH₂Cl₂ (30 mL) and dried over anhydrous MgSO₄ and the resulting residue was purified by flash chromatography (2% EtOAc/CH₂Cl₂) to afford the N—BOC quinazolinone intermediate (93.1 mg, 33.8%) as a solid. Under nitrogen, this N-Boc-intermediate (93.1 mg, 0.18 mmol) was dissolved in anhydrous CH₂Cl₂ (0.20 mL) and cooled to 0° C. using an ice-water bath. 4N HCl in dioxane (0.22 mL, 0.90 mmol) was added dropwise, and the reaction mixture was allowed to warm to rt. After stirring for 21 h, solvent was removed in vacuo. The residue was diluted with EtOAc (5 mL) and then washed with saturated Na₂CO₃ (8 mL). The layers were separated, and the aqueous phase was extracted with EtOAc (15 mL). The combined organic extracts were dried over anhydrous MgSO₄ and then filtered. The solvent was removed in vacuo, and the residue was purified by flash chromatography (2% MeOH/CH₂Cl₂) to afford (R)-6-nitro-2-(pyrrolidin-2-yl)-3-(3-(trifluoromethoxy)phenyl)quinazolin-4 (3H)-one (2jj) (40.6 mg, 54%) as a yellow solid. ¹H NMR (400 MHz, CDCl₃) δ 8.90 (dd, J=10.4, 2.6 Hz, 1H), 8.42 (ddd, J=8.9, 2.6, 1.3 Hz, 1H), 7.75 (dd, J=8.9, 4.5 Hz, 1H), 7.57-7.43 (m, 1H), 7.11 (ddt, J=8.5, 3.1, 1.6 Hz, 1H), 7.06-6.98 (m, 1H), 6.85-6.78 (m, 1H), 4.78 (dt, J=23.9, 7.8 Hz, 1H), 3.85 (d, J=13.1 Hz, 3H), 3.66-3.30 (m, 2H), 2.19-1.87 (m, 5H). MS (ESI) m/z: 421.0 (M+1)⁺

### Example 46. Synthesis of (S)-6-nitro-2-(pyrrolidin-2-yl)-3-(4-(trifluoromethoxy)phenyl)quinazolin-4 (3H)-one (2kk)

Prepared as shown in Scheme 1

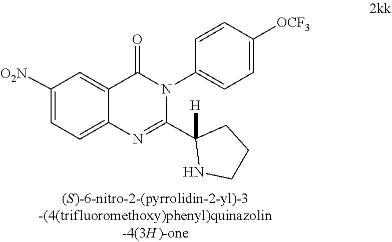 [SMILES: O=c1c2cc([N+](=O)[O-])ccc2nc([C@@H]2CCCN2)n1-c1ccc(OC(F)(F)F)cc1]

### Synthesis of (S)-6-nitro-2-(pyrrolidin-2-yl)-3-(4-(trifluoromethoxy)phenyl)quinazolin-4 (3H)-one (2kk)

To a solution of 5-nitroanthranilic acid (104.1 mg, 0.57 mmol) in dry pyridine (2 mL) was added Boc-L-proline (160.8 g, 0.75 mmol). After adding triphenyl phosphite (0.45 mL, 1.7 mmol), the reaction mixture was heated at 100° C. with MWI for 1 h. After cooling, 4-trifluoromethoxyaniline (0.09 mL, 0.68 mmol) was added, and the reaction mixture was heated at 60° C. with MWI for 15 min. The reaction mixture was diluted in CH₂Cl₂ and washed with 10% citric acid. The aqueous phase was washed with CH₂Cl₂ (30 mL) and dried over anhydrous MgSO₄ and the resulting residue was purified by flash chromatography (15% EtOAc/Hexanes) to afford the N—BOC quinazolinone intermediate (112.4 mg, 37.9%) as a solid. Under nitrogen, this N-Boc-intermediate (66.8 mg, 0.31 mmol) was dissolved in anhydrous 1,4-dioxane (0.30 mL) and cooled to 0° C. using an ice-water bath. To this cooled solution, 4N HCl in dioxane (0.27 mL, 1.08 mmol) was added dropwise, and the reaction mixture was allowed to warm to rt. After stirring for 16 h, solvent was removed in vacuo. The residue was diluted with EtOAc (5 mL) and then washed with saturated Na₂CO₃ (8 mL). The layers were separated, and the aqueous phase was extracted with EtOAc (15 mL). The combined organic extracts were dried over anhydrous MgSO₄ and then filtered. The solvent was removed in vacuo, and the residue was purified by flash chromatography (5% MeOH/CH₂Cl₂) to afford (S)-6-nitro-2-(pyrrolidin-2-yl)-3-(4-(trifluoromethoxy)phenyl)quinazolin-4 (3H)-one (2kk) (28.3 mg, 53%) as a gummy orange solid. ¹H NMR (400 MHz, CDCl₃) δ 9.07 (d, J=2.6 Hz, 1H), 8.54 (dd, J=8.9, 2.6 Hz, 1H), 7.82 (d, J=8.9 Hz, 1H), 7.42 (d, J=8.6 Hz, 2H), 7.40-7.35 (m, 1H), 7.32 (dt, J=7.7, 2.4 Hz, 1H), 3.82 (t, J=7.0 Hz, 1H), 3.27 (dt, J=10.6, 5.3 Hz, 1H), 2.81 (dt, J=10.3, 6.3 Hz, 1H), 2.73-2.61 (b, N—H, 1H), 1.90-1.67 (m, 4H). MS (ESI) m/z: 421.0 (M+1)⁺

### Example 47. Synthesis of (R)-3-(4-methoxyphenyl)-8-nitro-2-(pyrrolidin-2-yl)quinazolin-4 (3H)-one (2ll)

Prepared as shown in Scheme 1

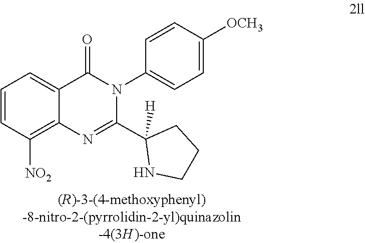 [SMILES: COc1ccc(-n2c([C@H]3CCCN3)nc3c([N+](=O)[O-])cccc3c2=O)cc1]

### Synthesis of (R)-3-(4-methoxyphenyl)-8-nitro-2-(pyrrolidin-2-yl)quinazolin-4 (3H)-one (2ll)

To a microwave vial containing 3-nitroanthranilic acid (91.0 mg, 0.5 mmol) and N-Boc-D-proline (0.14 g, 0.65 mmol) was added dry pyridine (1.5 mL). Triphenyl phosphite (0.4 mL, 1.5 mmol) was added to the vial, and the reaction mixture was heated at 100° C. with MWI for 80 min. After cooling the vial, 4-methoxyaniline (74.0 mg, 0.60 mmol) was added, and the reaction mixture was heated at 60° C. with MWI for 15 min. Pyridine was removed in vacuo, and the residue was diluted with CH₂Cl₂ (50 mL), and then the organic layer was washed sequentially with 10% citric acid (20 mL) and brine (50 mL). The separated organic extract was dried over MgSO₄, filtered, and concentrated in vacuo under reduced pressure. The crude mixture was purified by flash chromatography (0% Ethyl acetate/DCM˜3% Ethyl acetate/DCM) to afford N—BOC-quinazolinone as a yellow solid. The solid (94.6 mg, 0.203 mmol) was dissolved in dry dioxane (1.5 mL), cooled to 0° C., and 4 M HCl/dioxane (0.25 mL, 1.02 mmol) was added. After the addition was complete, the reaction mixture was stirred at rt for 8 h. Solvent was removed in vacuo under reduced pressure, and CH₂Cl₂ (10 mL) was added, followed by sequential washing with saturated Na₂CO₃ (5 mL) and brine (5 mL). The separated organic extract was dried over anhydrous MgSO₄, filtered and concentrated in vacuo under reduced pressure. The crude mixture was purified by flash chromatography (0% MeOH/CH₂Cl₂˜10% MeOH/CH₂Cl₂) to afford 2ll (31.4 mg, 17%) as yellow solid. ¹H NMR (500 MHz, Chloroform-d) δ 8.46 (dd, J=8.0, 1.5 Hz, 1H), 8.11 (dd, J=7.9, 1.5 Hz, 1H), 7.53 (t, J=7.9 Hz, 1H), 7.24-7.18 (m, 1H), 7.17-7.12 (m, 1H), 7.11-7.01 (m, 2H), 3.92 (t, J=7.1 Hz, 1H), 3.89 (s, 3H), 3.24 (dt, J=12.0, 6.2 Hz, 1H), 2.86-2.73 (m, 2H), 1.88-1.65 (m, 4H). MS (ESI) m/z: 367.1 (M+1)⁺.

### Example 48. Synthesis of (R)-3-(4-methoxyphenyl)-7-nitro-2-(pyrrolidin-2-yl)quinazolin-4 (3H)-one (2mm)

Prepared as shown in Scheme 1

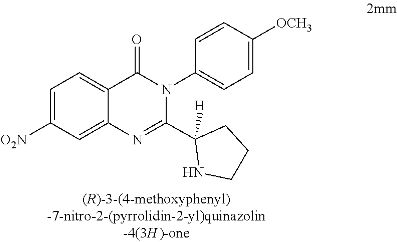 [SMILES: COc1ccc(-n2c([C@H]3CCCN3)nc3cc([N+](=O)[O-])ccc3c2=O)cc1]

### Synthesis of (R)-3-(4-methoxyphenyl)-7-nitro-2-(pyrrolidin-2-yl)quinazolin-4 (3H)-one (2 mm)

To a microwave vial containing 4-nitroanthranilic acid (91.0 mg, 0.5 mmol) and N-Boc-D-proline (0.14 g, 0.65 mmol) was added dry pyridine (1.5 mL). Triphenyl phosphite (0.4 mL, 1.5 mmol) was added to the vial, and the reaction mixture was heated at 100° C. with MWI for 80 min. After cooling the vial, 4-methoxyaniline (74.0 mg, 0.60 mmol) was added, and the reaction mixture was heated at 60° C. with MWI for 15 min. Pyridine was removed in vacuo, and the residue was diluted with CH₂Cl₂ (50 mL), and then the organic layer was washed sequentially with 10% citric acid (20 mL) and brine (50 mL). The separated organic extract was dried over MgSO₄, filtered, and concentrated in vacuo under reduced pressure. The crude mixture was purified by flash chromatography (0% Ethyl acetate/DCM˜3% Ethyl acetate/DCM) to afford the N—BOC quinazolinone as a yellow solid. The solid (0.137 g, 0.294 mmol) was dissolved in dry dioxane (1.5 mL), cooled to 0° C., and 4 M HCl/dioxane (0.37 mL, 1.47 mmol) was added. After the addition was complete, the reaction mixture was stirred at rt for 8 h. Solvent was removed in vacuo under reduced pressure, and CH₂Cl₂ (10 mL) was added, followed by sequential washing with saturated Na₂CO₃ (5 mL) and brine (5 mL). The separated organic extract was dried over anhydrous MgSO₄, filtered and concentrated in vacuo under reduced pressure. The crude mixture was purified by flash chromatography (0% MeOH/CH₂Cl₂-10% MeOH/CH₂Cl₂) to afford 2 mm (24.9 mg, 13%) as an off-yellow solid. ¹H NMR (500 MHz, Chloroform-d) δ 8.54 (d, J=2.2 Hz, 1H), 8.42 (d, J=8.7 Hz, 1H), 8.22 (dd, J=8.7, 2.2 Hz, 1H), 7.24-7.20 (m, 1H), 7.19-7.14 (m, 1H), 7.12-7.04 (m, 2H), 3.91 (d, J=8.0 Hz, 2H), 3.89 (s, 3H), 3.33-3.23 (m, 1H), 2.85-2.76 (m, 1H), 1.87-1.68 (m, 4H). MS (ESI) m/z: 367.1 (M+1)⁺.

### Example 49. Synthesis of (R)-3-(4-methoxyphenyl)-5-nitro-2-(pyrrolidin-2-yl)quinazolin-4 (3H)-one (2nn)

Prepared as shown in Scheme 1

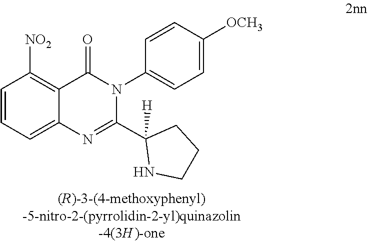 [SMILES: COc1ccc(-n2c([C@H]3CCCN3)nc3cccc([N+](=O)[O-])c3c2=O)cc1]

### Synthesis of (R)-3-(4-methoxyphenyl)-5-nitro-2-(pyrrolidin-2-yl)quinazolin-4 (3H)-one (2nn)

To a microwave vial containing 6-nitroanthranilic acid (91.0 mg, 0.5 mmol) and N-Boc-D-proline (0.14 g, 0.65 mmol) was added dry pyridine (1.5 mL). Triphenyl phosphite (0.4 mL, 1.5 mmol) was added to the vial, and the reaction mixture was heated at 100° C. with MWI for 80 min. After cooling the vial, 4-methoxyaniline (74.0 mg, 0.60 mmol) was added, and the reaction mixture was heated at 60° C. with MWI for 15 min. Pyridine was removed in vacuo, and the residue was diluted with CH₂Cl₂ (50 mL), and then the organic layer was washed sequentially with 10% citric acid (20 mL) and brine (50 mL). The separated organic extract was dried over MgSO₄, filtered, and concentrated in vacuo under reduced pressure. The crude mixture was purified by flash chromatography (0% Ethyl acetate/DCM˜3% Ethyl acetate/DCM) to afford N—BOC quinazolinone as a yellow solid. The solid (35.5 mg, 0.0761 mmol) was dissolved in dry dioxane (1.5 mL), cooled to 0° C., and 4 M HCl/Dioxane (0.10 mL, 0.38 mmol) was added. After the addition was complete, the reaction mixture was stirred at rt for 8 h. Solvent was removed in vacuo under reduced pressure, and CH₂Cl₂ (10 mL) was added, followed by sequential washing with saturated Na₂CO₃ (5 mL) and brine (5 mL). The separated organic extract was dried over anhydrous MgSO₄, filtered and concentrated in vacuo under reduced pressure. The crude mixture was purified by flash chromatography (0% MeOH/CH₂Cl₂˜10% MeOH/CH₂Cl₂) to afford 2nn (13.2 mg, 7%) as an off-yellow solid. ¹H NMR (500 MHz, Chloroform-d) δ 7.86 (dd, J=8.4, 1.2 Hz, 1H), 7.81 (t, J=7.9 Hz, 1H), 7.45 (dd, J=7.6, 1.2 Hz, 1H), 7.21-7.12 (m, 2H), 7.07-6.99 (m, 2H), 3.89 (t, J=7.3 Hz, 1H), 3.86 (s, 3H), 3.26 (dt, J=11.4, 5.8 Hz, 1H), 2.86-2.72 (m, 1H), 1.88-1.64 (m, 4H). MS (ESI) m/z: 367.1 (M+1)⁺.

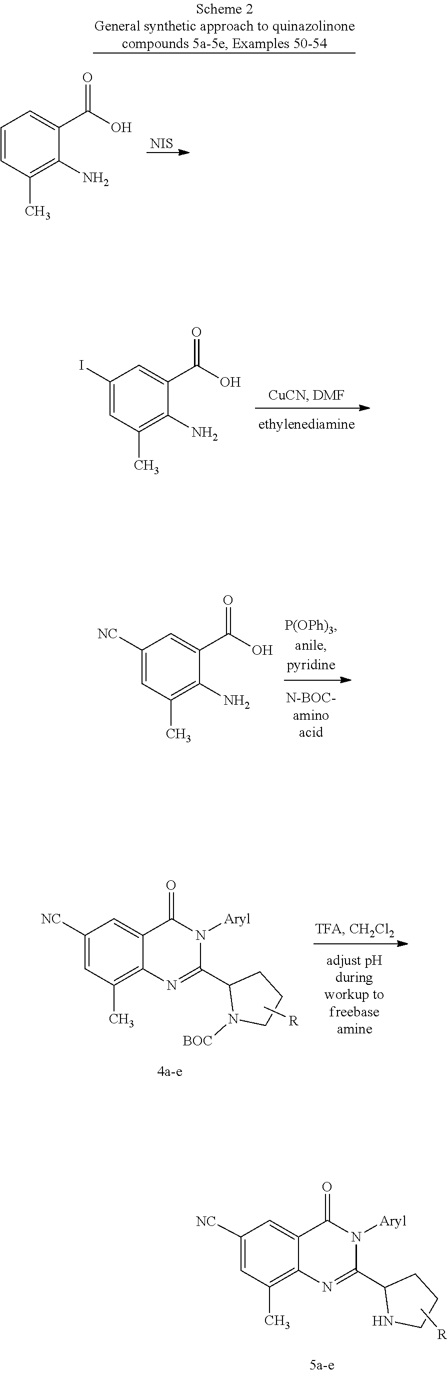

### Example 50. Synthesis of (R)-3-(4-methoxyphenyl)-8-methyl-4-oxo-2-(pyrrolidin-2-yl)-3,4-dihydroquinazoline-6-carbonitrile (5a)

Prepared as shown in Scheme 2

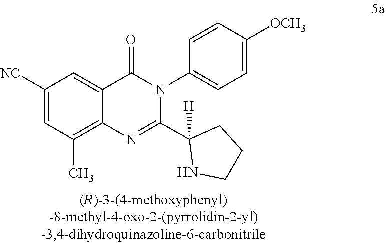 [SMILES: COc1ccc(-n2c([C@H]3CCCN3)nc3c(C)cc(C#N)cc3c2=O)cc1]

Step 1. Synthesis of 2-amino-5-iodo-3-methylbenzoic acid

A mixture of 2-amino-3-methyl-benzoic acid (3.02 g, 20.00 mmol) and N-iodosuccinimide (4.75 g, 21.00 mmol) in DMF (50 mL) was heated at 75° C. for 4 h. After cooling to room temperature, the mixture was poured into ice/water/NH₄Cl solution and the resulting precipitate was collected by filtration. After washing the residue with water, the resulting solid was dissolved with acetone, then dried over with anhydrous Na₂SO₄, and filtered, then the solvent was evaporated under reduced pressure to afford 2-amino-5-iodo-3-methylbenzoic acid (4.15 g, 75.1%) as brown solid. ¹H NMR (400 MHz, DMSO-d₆) δ 7.86 (d, J=1.8 Hz, 1H), 7.45 d, J=2.5 Hz, 1H), 2.05 (s, 3H). MS (ESI) m/z: 278.1 (M+1)⁺.

Step 2. Synthesis of 2-amino-5-cyano-3-methylbenzoic acid

A mixture of 2-amino-5-iodo-3-methylbenzoic acid (2.21 g, 8.00 mmol) and copper cyanide (0.93 g, 10.40 mmol) in DMF (30 mL) was heated at 150° C. for 16 h. After cooling to room temperature, and most of DMF was removed by rotavapor, water (30 mL) was added to reaction mixture, then followed by ethylenediamine (3 mL), and the mixture was stirred for 1 h. The solids were removed by filtration, then reaction mixture was adjusted to pH 5 with concentrated hydrochloric acid, then extracted with ethyl acetate (2×200 mL) and water (30 mL). The combined organic layer was dried over with anhydrous Na₂SO₄, and filtered, then the solvent was evaporated under reduced pressure to afford 2-amino-5-cyano-3-methylbenzoic acid (0.65 g, 46.2%) as brown solid. ¹H NMR (400 MHz, DMSO-d₆) δ 7.95 (d, J=1.8 Hz, 1H), 7.52 d, J=1.9 Hz, 1H), 7.28 (2H, brs), 2.12 (s, 3H). MS (ESI) m/z: 177.1 (M+1)⁺.

Step 3. Synthesis of tert-butyl (R)-2-(6-cyano-3-(4-methoxyphenyl)-8-methyl-4-oxo-3,4-dihydroquinazolin-2-yl) pyrrolidine-1-carboxylate (4a). 2-Amino-5-cyano-3-methylbenzoic acid (116.00 mg, 0.66 mmol) and Boc-D-proline (0.28 g, 1.32 mmol) were dissolved in dry pyridine (3 mL). Triphenyl phosphite (0.51 mL, 1.95 mmol) was added to the mixture and the reaction mixture was heated at 100° C. with MWI for 40 min. 4-methoxyaniline (0.32 g, 2.65 mmol) was added to the mixture and the reaction mixture was heated at 100° C. with MWI for 10 min. Pyridine was removed in vacuo and the residue was diluted with CH₂Cl₂ (100 mL) and washed with 1N aqueous HCl (50 mL). The organic layer was subsequently washed with H₂O (50 mL) and then brine (50 mL). The separated organic extract was dried over anhydrous Na₂SO₄. Once filtered, the solvent was evaporated under reduced pressure. The residue was purified by flash chromatography (0% Ethyl acetate/CH₂Cl₂˜5% Ethyl acetate/CH₂Cl₂) to afford 4a (116.85 mg, 38.5%) as off-white solid. ¹H NMR (500 MHz, CDCl₃) δ 8.42 (dd, J=11.7, 1.4 Hz, 1H), 7.76 (d, J=21.8, 1H), 7.46-7.15 (m, 1H), 7.15-7.01 (m, 3H), 4.55-4.45 (m, 1H), 3.88 (d, J=11.1 Hz, 3H), 3.80-3.70 (m, 1H), 3.59-3.45 (m, 1H), 2.58 (s, 3H), 2.10-1.82 (m, 4H), 1.43 (s, 4H), 1.26 (d, J=4.3 Hz, 5H). MS (ESI) m/z: 461.2 (M+1)⁺.

Step 4. Synthesis of (R)-3-(4-methoxyphenyl)-8-methyl-4-oxo-2-(pyrrolidin-2-yl)-3,4-dihydroquinazoline-6-carbonitrile (5a)

Under nitrogen, trifluoroacetic acid (0.19 mL, 2.60 mmol) was added to a solution of 4a (60.00 mg, 0.13 mmol) in dry CH₂Cl₂ (5 mL) at 0° C. The solution was stirred at rt overnight, and then the solvent was removed in vacuo. The residue was diluted with CH₂Cl₂ (20 mL), washed sequentially with saturated solutions of Na₂CO₃ (10 mL) and brine (10 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 5a (26.80 mg, 57.5%) as off-white solid. ¹H NMR (500 MHz, CDCl₃) δ 8.42 (s, 1H), 7.78 (s, 1H), 7.23-7.13 (m, 2H), 7.06 (d, J=8.9 Hz, 2H), 3.91 (d, J=7.5 Hz, 1H), 3.88 (s, 3H), 3.30-3.24 (m, 1H), 2.96-2.89 (m, 1H), 2.85-2.80 (m, 1H), 2.64 (s, 3H), 1.85-1.75 (m, 4H). MS (ESI) m/z: 361.1 (M+1)⁺.

### Example 51. Synthesis of (S)-3-(4-methoxyphenyl)-8-methyl-4-oxo-2-(pyrrolidin-2-yl)-3,4-dihydroquinazoline-6-carbonitrile (5b)

Prepared as shown in Scheme 2

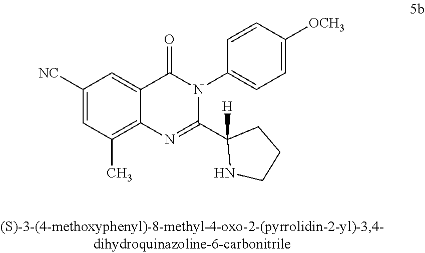 [SMILES: COc1ccc(-n2c([C@@H]3CCCN3)nc3c(C)cc(C#N)cc3c2=O)cc1]

Step 1. Synthesis of tert-butyl (S)-2-(6-cyano-3-(4-methoxyphenyl)-8-methyl-4-oxo-3,4-dihydroquinazolin-2-yl)pyrrolidine-1-carboxylate (4b). 2-amino-5-cyano-3-methylbenzoic acid (176.18 mg, 1.00 mmol) and Boc-L-proline (0.28 g, 1.30 mmol) were dissolved in dry pyridine (4 mL). Triphenyl phosphite (0.79 mL, 3.00 mmol) was added to the mixture and the reaction mixture was heated at 100° C. with MWI for 40 min. 4-methoxyaniline (148.00 mg, 1.20 mmol) was added to the mixture and the reaction mixture was heated at 100° C. with MWI for 10 min. Pyridine was removed in vacuo and the residue was diluted with CH₂Cl₂ (100 mL) and washed with 1N HCl aqueous (60 mL). The organic layer was washed with H₂O (60 mL) and brine (60 mL), dried over anhydrous Na₂SO₄, and filtered, then the solvent was evaporated under reduced pressure. The residue was purified by flash chromatography (0% Ethyl acetate/CH₂Cl₂˜10% Ethyl acetate/CH₂Cl₂) to afford 4b (0.21 g, 45.6%) as off-white solid. ¹H NMR (400 MHz, CDCl₃) δ 8.34 (dd, J=10.5, 1.3 Hz, 1H), 7.69 (d, J=17.9 Hz, 1H), 7.41-7.12 (m, 1H), 7.08-6.93 (m, 4H), 4.51-4.46 (m, 1H), 3.81 (d, J=8.9 Hz, 3H), 3.72-3.66 (m, 1H), 3.52-3.31 (m, 1H), 2.51 (s, 3H), 2.04-1.75 (m, 4H), 1.36 (s, 4H), 1.19 (s, 5H). MS (ESI) m/z: 461.2 (M+1)⁺.

Step 2. Synthesis of (S)-3-(4-methoxyphenyl)-8-methyl-4-oxo-2-(pyrrolidin-2-yl)-3,4-dihydroquinazoline-6-carbonitrile (5b)

Under nitrogen, trifluoroacetic acid (0.50 mL, 7.00 mmol) was added to a solution of 4b (160.00 mg, 0.35 mmol) in dry CH₂Cl₂ (6 mL) at 0° C. The solution was stirred at rt overnight, and then the solvent was removed in vacuo. The residue was diluted with CH₂Cl₂ (20 mL), washed sequentially with saturated solutions of Na₂CO₃ (10 mL) and brine (10 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 5b (74.33 mg, 58.9%) as off-white solid. ¹H NMR (500 MHz, CDCl₃) δ 8.40 (s, 1H), 7.77 (s, 1H), 7.21-7.14 (m, 2H), 7.06 (d, J=8.8 Hz, 2H), 3.93-3.87 (m, 4H), 3.32-3.25 (m, 1H), 2.84-2.78 (m, 1H), 2.68-2.63 (m, 4H), 1.84-1.72 (m, 4H). MS (ESI) m/z: 361.1 (M+1)⁺.

### Example 52. Synthesis of (R)-3-(3-fluoro-4-methoxyphenyl)-8-methyl-4-oxo-2-(pyrrolidin-2-yl)-3,4-dihydroquinazoline-6-carbonitrile (5c)

Prepared as shown in Scheme 2

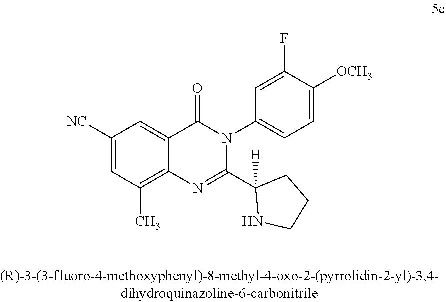 [SMILES: COc1ccc(-n2c([C@H]3CCCN3)nc3c(C)cc(C#N)cc3c2=O)cc1F]

Step 1. Synthesis of tert-butyl (R)-2-(6-cyano-3-(3-fluoro-4-methoxyphenyl)-8-methyl-4-oxo-3,4-dihydroquinazolin-2-yl)pyrrolidine-1-carboxylate (4c). 2-amino-5-cyano-3-methylbenzoic acid (88.09 mg, 0.50 mmol) and Boc-D-proline (0.14 g, 0.65 mmol) were dissolved in dry pyridine (3 mL). Triphenyl phosphite (0.40 mL, 1.50 mmol) was added to the mixture and the reaction mixture was heated at 100° C. with MWI for 40 min. 3-Fluoro-4-methoxyaniline (84.7 mg, 0.60 mmol) was added to the mixture and the reaction mixture was heated at 100° C. with MWI for 2×20 min. Pyridine was removed in vacuo and the residue was diluted with CH₂Cl₂ (60 mL) and washed with 1N aqueous HCl (20 mL). The organic layer was washed with H₂O (20 mL) and brine (20 mL), dried over anhydrous Na₂SO₄, and filtered, then the solvent was evaporated under reduced pressure. The residue was purified by flash chromatography (0% Ethyl acetate/Hexane˜40% Ethyl acetate/Hexane) to afford 4c (29.50 mg, 12.4%) as off-white solid. ¹H NMR (500 MHz, CDCl₃) δ 8.40 (d, J=13.7 Hz, 1H), 7.77 (d, J=23.8 Hz, 1H), 7.38-7.06 (m, 2H), 6.99 (dt, J=19.2, 6.2 Hz, 1H), 4.65-4.46 (m, 1H), 3.98 (dd, J=11.8, 2.8 Hz, 3H), 3.83-3.67 (m, 1H), 3.59-3.44 (m, 1H), 2.58 (s, 3H), 2.16-1.84 (m, 4H), 1.42 (d, J=4.8 Hz, 5H), 1.28 (t, J=8.6 Hz, 4H). MS (ESI) m/z: 479.1 (M+1)⁺.

Step 2. Synthesis of (R)-3-(3-fluoro-4-methoxyphenyl)-8-methyl-4-oxo-2-(pyrrolidin-2-yl)-3,4-dihydroquinazoline-6-carbonitrile (5c)

Under nitrogen, trifluoroacetic acid (0.04 mL, 0.54 mmol) was added to a solution of 4c (26.00 mg, 0.054 mmol) in dry CH₂Cl₂ (1 mL) y at 0° C. The solution was stirred at rt overnight, and then the solvent was removed in vacuo. The residue was diluted with CH₂Cl₂ (20 mL), washed sequentially with saturated solutions of Na₂CO₃ (10 mL) and brine (10 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 5c (7.10 mg, 34.8%) as pale yellow solid. ¹H NMR (500 MHz, CDCl₃) δ 8.42 (s, 1H), 7.80 (s, 1H), 7.12 (t, J=8.2 Hz, 1H), 7.08-6.97 (m, 2H), 3.98 (s, 3H), 3.90 (dd, J=14.4, 7.0 Hz, 1H), 3.28 (dd, J=10.7, 5.0 Hz, 1H), 2.83 (dd, J=10.3, 4.0 Hz, 1H), 2.64 (s, 3H), 2.04 (s, 2H), 1.85-1.75 (m, 4H). MS (ESI) m/z: 379.1 (M+1)⁺.

### Example 53. Synthesis of 2-((2R,4S)-4-fluoropyrrolidin-2-yl)-3-(4-methoxyphenyl)-8-methyl-4-oxo-3,4-dihydroquinazoline-6-carbonitrile (5d)

Prepared as shown in Scheme 2.

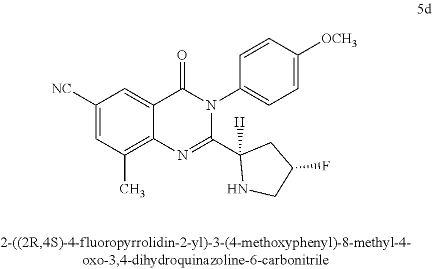 [SMILES: COc1ccc(-n2c([C@H]3C[C@H](F)CN3)nc3c(C)cc(C#N)cc3c2=O)cc1]

Step 1. Synthesis of (2R,4S)-1-(tert-butoxycarbonyl)-4-fluoropyrrolidine-2-carboxylic acid. To a solution of (2R,4S)-4-fluoropyrrolidine-2-carboxylic acid (0.50 g, 3.76 mmol), di-tert-butyl dicarbonate (1.23 g, 5.63 mmol) in mL THE (5 ml) was added the solution of sodium hydroxide (0.17 g, 4.23 mmol) in H₂O (2.5 ml) at 0° C., then stirred at rt for 8 hours. The mixture was then adjusted to pH 2 with 1.0N HCl and extracted with ethyl acetate (2×30 ml). The combined organic layers were washed with brine and dried over anhydrous Na₂SO₄, then concentrated under reduced pressure to afford (2R,4S)-1-(tert-butoxycarbonyl)-4-fluoropyrrolidine-2-carboxylic acid (0.86 g, 98.1%) as pale orange oil. ¹H NMR (400 MHz, CDCl₃) δ 5.31-5.18 (m, 1H), 4.51-4.38 (m, 1H), 3.95-3.88 (m, 1H), 3.65-3.56 (m, 1H), 2.74-2.53 (m, 1H), 2.47-2.07 (m, 1H), 1.49 (s, 5H), 1.44 (s, 4H). MS (ESI) m/z: 234.1 (M+1)⁺.

Step 2. Synthesis of tert-butyl (2R,4S)-2-(6-cyano-3-(4-methoxyphenyl)-8-methyl-4-oxo-3,4-dihydroquinazol-in-2-yl)-4-fluoropyrrolidine-1-carboxylate (4d). 2-Amino-5-cyano-3-methylbenzoic acid (44.05 mg, 0.25 mmol) and (2R,4S)-1-(tert-butoxycarbonyl)-4-fluoropyrrolidine-2-carboxylic acid (75.80 mg, 0.33 mmol) were dissolved in dry pyridine. Triphenyl phosphite (0.19 mL, 0.75 mmol) was added to the mixture and the reaction mixture was heated at 100° C. with MWI for 40 min. 4-methoxyaniline (36.96 mg, 0.30 mmol) was added to the mixture and the reaction mixture was heated at 100° C. with MWI for 20 min. Pyridine was removed in vacuo and the residue was diluted with CH₂Cl₂ (30 mL) and washed with 1N HCl aqueous (20 mL). The organic layer was washed with H₂O (30 mL) and brine (30 mL), dried over anhydrous Na₂SO₄, and filtrated, then the solvent was evaporated under reduced pressure. The residue was purified by flash chromatography (0% Ethyl acetate/Hexane to 30% Ethyl acetate/Hexane) to afford 4d (56.65 mg, 47.5%) as colorless oil. ¹H NMR (400 MHz, CDCl₃) δ 8.33 (dd, J=12.6, 1.3 Hz, 1H), 7.69 (d, J=20.2 Hz, 1H), 7.45-7.16 (m, 1H), 7.06-6.86 (m, 3H), 5.24-5.12 (m, 1H), 4.68-4.45 (m, 1H), 3.86-3.75 (s, 5H), 2.49 (s, 3H), 2.28-2.05 (m, 2H), 1.35 (s, 5H), 1.17 (s, 4H). MS (ESI) m/z: 479.1 (M+1)⁺.

Step 3. Synthesis of 2-((2R,4S)-4-fluoropyrrolidin-2-yl)-3-(4-methoxyphenyl)-8-methyl-4-oxo-3,4-dihydroquinazoline-6-carbonitrile (5d)

Under nitrogen, trifluoroacetic acid (0.16 mL, 2.08 mmol) was added to a solution of 4d (50.00 mg, 0.10 mmol) in dry CH₂Cl₂ (2 mL) at 0° C. The solution was stirred at rt overnight, and then the solvent was removed in vacuo. The residue was diluted with CH₂Cl₂ (30 mL), washed sequentially with saturated solutions of Na₂CO₃ (10 mL) and brine (10 mL), and the separated organic extract was dried over anhydrous Na₂SO₄. Once filtered, the solvent was evaporated under reduced pressure. The residue was purified by flash chromatography (0% MeOH/CH₂Cl₂˜5% MeOH/CH₂Cl₂) to afford 5d (26.40 mg, 69.8%) as pale yellow solid. ¹H NMR (400 MHz, CDCl₃) δ 8.35 (d, J=1.5 Hz, 1H), 7.71 (d, J=1.2 Hz, 1H), 7.21 (t, J=2.6 Hz, 1H), 7.09-7.05 (m, 1H), 7.03-6.98 (m, 2H), 5.23 (dt, J=54.3, 4.1 Hz, 1H), 4.19 (t, J=7.6 Hz, 1H), 3.82 (s, 3H), 3.39-3.24 (m, 1H), 3.18-3.08 (m, 1H), 2.55 (s, 3H), 2.31-2.19 (m, 1H), 2.15-2.04 (m, 1H). MS (ESI) m/z: 379.1 (M+1)⁺.

### Example 54. Synthesis of 3-(3-fluoro-4-methoxyphenyl)-2-((2R,4S)-4-fluoropyrrolidin-2-yl)-8-methyl-4-oxo-3,4-dihydroquinazoline-6-carbonitrile (5e)

Prepared as shown in Scheme 2.

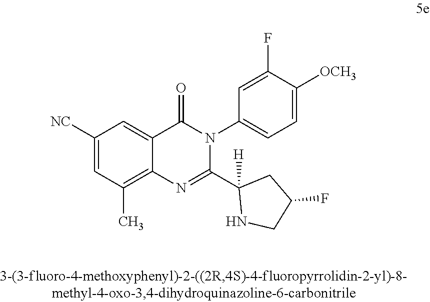 [SMILES: COc1ccc(-n2c([C@H]3C[C@H](F)CN3)nc3c(C)cc(C#N)cc3c2=O)cc1F]

Step 1. Synthesis of tert-butyl (2R,4S)-2-(6-cyano-3-(3-fluoro-4-methoxyphenyl)-8-methyl-4-oxo-3,4-dihydroquinazol-in-2-yl)-4-fluoropyrrolidine-1-carboxylate (4e). 2-amino-5-cyano-3-methylbenzoic acid (35.24 mg, 0.20 mmol) and (2R,4S)-1-(tert-butoxycarbonyl)-4-fluoropyrrolidine-2-carboxylic acid 60.64 mg, 0.26 mmol) were dissolved in dry pyridine (3 mL). Triphenyl phosphite (0.16 mL, 0.60 mmol) was added to the mixture and the reaction mixture was heated at 100° C. with MWI for 40 min. 3-Fluoro-4-methoxyaniline (33.88 mg, 0.24 mmol) was added to the mixture and the reaction mixture was heated at 100° C. with MWI for 20 min. Pyridine was removed in vacuo and the residue was diluted with CH₂Cl₂ (30 mL) and washed with 1N HCl aqueous (20 mL). The organic layer was washed with H₂O (30 mL) and brine (30 mL), dried over anhydrous Na₂SO₄, and filtered, then the solvent was evaporated under reduced pressure. The residue was purified by flash chromatography (0% Ethyl acetate/CH₂Cl₂˜ 30% Ethyl acetate/CH₂Cl₂) to afford 4e (25.30 mg, 25.5%) as pale yellow oil. ¹H NMR (500 MHz, CDCl₃) δ 8.33 (d, J=14.8 Hz, 1H), 7.70 (d, J=25.7 Hz, 1H), 7.38-7.01 (m, 2H), 6.92-6.81 (m, 1H), 5.17 (dd, J=52.7, 15.2 Hz, 1H), 4.65-4.58 (m, 1H), 3.98-3.69 (m, 5H), 2.49 (s, 3H), 2.36-2.10 (m, 2H), 1.34 (d, J=5.1 Hz, 5H), 1.17 (d, J=1.8 Hz, 4H). MS (ESI) m/z: 497.1 (M+1)⁺.

Step 2. Synthesis of 3-(3-fluoro-4-methoxyphenyl)-2-((2R,4S)-4-fluoropyrrolidin-2-yl)-8-methyl-4-oxo-3,4-dihydroquinazoline-6-carbonitrile (5e)

Under nitrogen, trifluoroacetic acid (0.07 mL, 1.00 mmol) was added to a solution of 4e (25.00 mg, 0.05 mmol) in dry CH₂Cl₂ (1 mL) at 0° C., then stirred at room temperature overnight, then the solvent was removed in vacuo and the residue was diluted with CH₂Cl₂ (20 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 5e (11.18 mg, 56.0%) as pale yellow solid. ¹H NMR (400 MHz, CDCl₃) δ 8.35 (s, 1H), 7.73 (s, 1H), 7.11-7.02 (m, 2H), 6.92 (dd, J=15.4, 5.5 Hz, 1H), 5.25 (dt, J=54.4, 3.8 Hz, 1H), 4.17 (q, J=7.5 Hz, 1H), 3.91 (s, 3H), 3.35-3.06 (m, 2H), 2.55 (s, 3H), 2.43-2.23 (m, 1H), 2.19-2.06 (m, 1H). MS (ESI) m/z: 397.1 (M+1)⁺.

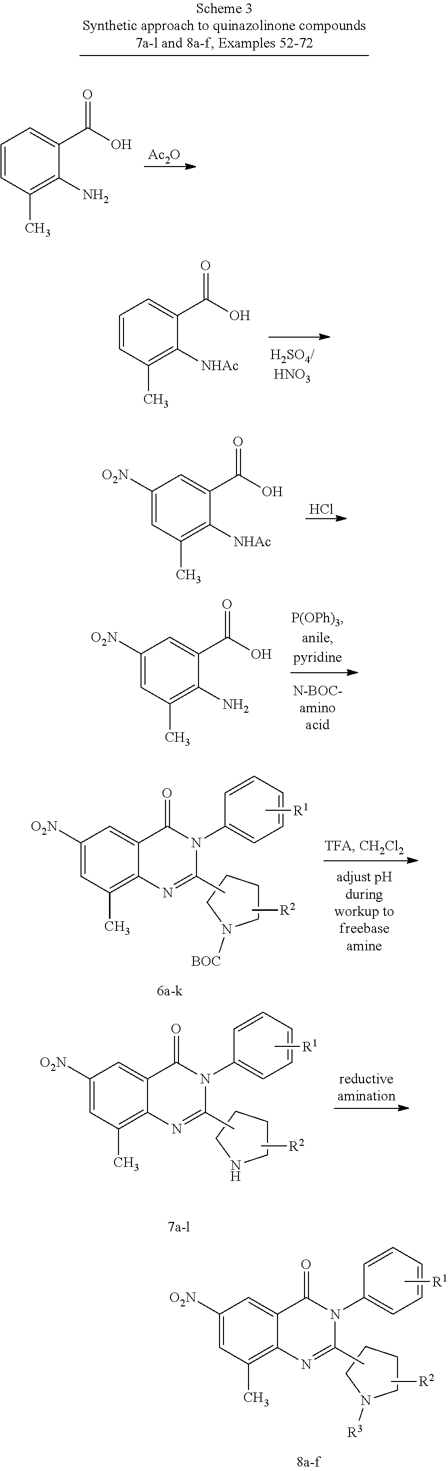

### Example 55. Synthesis of (R)-3-(4-methoxyphenyl)-8-methyl-6-nitro-2-(pyrrolidin-2-yl) quinazolin-4 (3H)-one (7a)

Prepared according to Scheme 3.

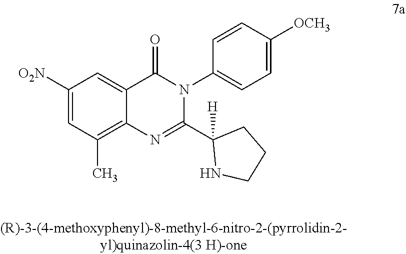 [SMILES: COc1ccc(-n2c([C@H]3CCCN3)nc3c(C)cc([N+](=O)[O-])cc3c2=O)cc1]

Step 1. Synthesis of 2-acetoamido-3-methyl-benzoic acid. Acetic anhydride (18.65 mL, 198.45 mmol) was added to the 2-amino-3-methylbenzoic acid (10.00 g, 66.15 mmol), then the resulting suspension was heated to reflux for 2 h. After cooling to room temperature, the mixture was poured into ice/water and the suspension was stirred for 2 h, then the resulting precipitate was collected by filtration. After washing the residue with water, the resulting solid was dissolved with acetone, then dried over with anhydrous Na₂SO₄, and filtered, then the solvent was evaporated under reduced pressure to afford 2-acetoamido-3-methyl-benzoic acid (11.96 g, 94.0%) as light brown solid. ¹H NMR (400 MHz, DMSO) δ 9.48 (brs, 1H), 7.59 (d, J=7.1 Hz, 1H), 7.42 (d, J=7.3 Hz, 1H), 7.22 (t, J=7.6 Hz, 1H), 2.20 (s, 3H), 2.01 (s, 3H). MS (ESI) m/z: 194.1 (M+1)⁺.

Step 2. Synthesis of 2-acetamido-3-methyl-5-nitrobenzoic acid. 2-acetoamido-3-methyl-benzoic acid (9.00 g, 46.64 mmol) was dissolved in concentrated H₂SO₄ (120 mL), and the resulting mixture was cooled to 0° C. Fuming HNO₃ (13.04 mL, 279.85 mmol) was slowly added, then the mixture was stirred at 0° C. for 12 h. The mixture was poured into ice/water and the suspension was stirred for 2 h, then the resulting precipitate was collected by filtration. After washing the residue with water, the resulting solid was dissolved with acetone, then dried over with anhydrous Na₂SO₄, and filtered, then the solvent was evaporated under reduced pressure to afford 2-acetamido-3-methyl-5-nitrobenzoic acid (9.60 g, 86.6%) as off-white solid. ¹H NMR (400 MHz, DMSO) δ 9.97 (s, 1H), 8.31 (s, 2H), 2.35 (s, 3H), 2.07 (s, 3H). MS (ESI) m/z: 239.1 (M+1)⁺.

Step 3. Synthesis of 2-amino-3-methyl-5-nitrobenzoic acid. 2-acetamido-3-methyl-5-nitrobenzoic acid (2.80 g, 11.76 mmol) was dissolved in concentrated HCl (60 mL), and the solution was refluxed for 4 h. After cooling to room temperature, the mixture was poured into ice/water and the suspension was stirred for 2 h, then the resulting precipitate was collected by filtration. After washing the residue with water, the resulting solid was dissolved with acetone, then dried over with anhydrous Na₂SO₄, and filtered, then the solvent was evaporated under reduced pressure to afford 2-amino-3-methyl-5-nitrobenzoic acid (1.75 g, 75.6%) as pale yellow solid. ¹H NMR (400 MHz, DMSO) δ 13.28 (brs, 1H), 8.53 (d, J=2.7 Hz, 1H), 8.02 (d, J=2.4 Hz, 1H), 7.72 (brs, 2H), 2.20 (s, 3H). MS (ESI) m/z: 197.1 (M+1)⁺.

Step 4. Synthesis of tert-butyl (R)-2-(3-(4-methoxyphenyl)-8-methyl-6-nitro-4-oxo-3,4-dihydroquinazolin-2-yl) pyrrolidine-1-carboxylate (6a). 2-amino-3-methyl-5-nitrobenzoic acid (392.00 mg, 2.00 mmol) and Boc-D-proline (0.56 g, 2.60 mmol) were dissolved in dry pyridine (6 mL). Triphenyl phosphite (1.58 mL, 6.00 mmol) was added to the mixture and the reaction mixture was heated at 100° C. with MWI for 40 min. 4-methoxyaniline (295.58 mg, 2.40 mmol) was added to the mixture and the reaction mixture was heated at 100° C. with MWI for 20 min. Pyridine was removed in vacuo and the residue was diluted with CH₂Cl₂ (160 mL) and washed with 1N HCl aqueous (80 mL). The organic layer was washed with H₂O (80 mL) and brine (80 mL), dried over anhydrous Na₂SO₄, and filtered, then the solvent was evaporated under reduced pressure. The residue was purified by flash chromatography (0% Ethyl acetate/Hexane˜40% Ethyl acetate/Hexane) to afford 6a (0.48 g, 50.0%) as pale yellow solid. ¹H NMR (500 MHz, CDCl₃) δ 8.93 (dd, J=14.1, 2.3 Hz, 1H), 8.38 (dd, J=20.7, 1.9 Hz, 1H), 7.48-7.18 (m, 1H), 7.18-7.03 (m, 3H), 4.61-4.56 (m, 1H), 3.89 (d, J=11.2 Hz, 3H), 3.82-3.71 (m, 1H), 3.56-3.48 (m, 1H), 2.64 (s, 3H), 2.16-1.84 (m, 4H), 1.43 (s, 4H), 1.26 (s, 5H). MS (ESI) m/z: 481.1 (M+1)⁺.

Step 5. Synthesis of (R)-3-(4-methoxyphenyl)-8-methyl-6-nitro-2-(pyrrolidin-2-yl) quinazolin-4 (3H)-one (7a)

Under nitrogen, trifluoroacetic acid (1.40 mL, 18.40 mmol) was added to a solution of 6a (0.44 g, 0.92 mmol) in dry CH₂Cl₂ (30 mL) at 0° C., then stirred at room temperature overnight, then the solvent was removed in vacuo and the residue was diluted with CH₂Cl₂ (40 mL) and washed with saturated solutions of Na₂CO₃ (20 mL) and brine (20 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 7a (0.15 g, 42.9%) as pale yellow solid. ¹H NMR (400 MHz, CDCl₃) δ 8.95 (d, J=2.3 Hz, 1H), 8.40 (d, J=1.7 Hz, 1H), 7.23-7.15 (m, 2H), 7.07 (d, J=9.1 Hz, 2H), 3.94-3.85 (m, 4H), 3.29 (dd, J=11.0, 5.0 Hz, 1H), 2.82 (dt, J=13.6, 6.8 Hz, 1H), 2.70 (s, 3H), 1.87-1.70 (m, 4H). MS (ESI) m/z: 381.1 (M+1)⁺.

### Example 56. Synthesis of (S)-3-(4-methoxyphenyl)-8-methyl-6-nitro-2-(pyrrolidin-2-yl) quinazolin-4 (3H)-one (7b)

Prepared according to Scheme 3.

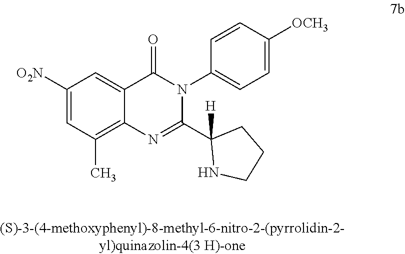 [SMILES: COc1ccc(-n2c([C@@H]3CCCN3)nc3c(C)cc([N+](=O)[O-])cc3c2=O)cc1]

Step 1. Synthesis of tert-butyl (S)-2-(3-(4-methoxyphenyl)-8-methyl-6-nitro-4-oxo-3,4-dihydroquinazolin-2-yl) pyrrolidine-1-carboxylate (6b). 2-amino-3-methyl-5-nitrobenzoic acid (156.93 mg, 0.80 mmol) and Boc-L-proline (223.86 mg, 1.04 mmol) were dissolved in dry pyridine (5 mL). Triphenyl phosphite (0.63 mL, 2.40 mmol) was added to the mixture and the reaction mixture was heated at 100° C. with MWI for 40 min. 4-methoxyaniline (118.23 mg, 0.96 mmol) was added to the mixture and the reaction mixture was heated at 100° C. with MWI for 20 min. Pyridine was removed in vacuo and the residue was diluted with CH₂Cl₂ (100 mL) and washed with 1N HCl aqueous (60 mL). The organic layer was washed with H₂O (60 mL) and brine (60 mL), dried over anhydrous Na₂SO₄, and filtered, then the solvent was evaporated under reduced pressure. The residue was purified by flash chromatography (0% Ethyl acetate/Hexane˜50% Ethyl acetate/Hexane) to afford 6b (0.18 g, 46.8%) as pale yellow solid. ¹H NMR (400 MHz, CDCl₃) δ 8.92 (dd, J=12.0, 2.5 Hz, 1H), 8.37 (dd, J=17.1, 2.1 Hz, 1H), 7.48-7.21 (m, 1H), 7.18-7.02 (m, 3H), 4.61-4.52 (m, 1H), 3.89 (d, J=8.8 Hz, 3H), 3.85-3.71 (m, 1H), 3.60-3.46 (m, 1H), 2.64 (s, 3H), 2.14-1.84 (m, 4H), 1.43 (s, 4H), 1.26 (s, 5H). MS (ESI) m/z: 481.1 (M+1)⁺.

Step 2. Synthesis of (S)-3-(4-methoxyphenyl)-8-methyl-6-nitro-2-(pyrrolidin-2-yl) quinazolin-4 (3H)-one (7b)

Under nitrogen, trifluoroacetic acid (0.55 mL, 7.50 mmol) was added to a solution of 6b (0.18 g, 0.37 mmol) in dry CH₂Cl₂ (10 mL) at 0° C., then stirred at room temperature overnight, then the solvent was removed in vacuo and the residue was diluted with CH₂Cl₂ (30 mL) and washed with saturated solutions of Na₂CO₃ (20 mL) and brine (20 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 7b (0.12 g, 85.2%) as pale yellow solid. ¹H NMR (400 MHz, CDCl₃) δ 8.95 (d, J=2.6 Hz, 1H), 8.42 (d, J=1.6 Hz, 1H), 7.23-7.15 (m, 2H), 7.09-7.05 (m, 2H), 3.96-3.82 (m, 4H), 3.29 (dt, J=15.0, 6.1 Hz, 1H), 2.82 (dt, J=10.8, 6.7 Hz, 1H), 2.70 (s, 3H), 1.86-1.72 (m, 4H). MS (ESI) m/z: 381.1 (M+1)⁺.

### Example 57. Synthesis of (R)-3-(4-methoxyphenyl)-8-methyl-2-(1-methylpyrrolidin-2-yl)-6-nitroquinazolin-4 (3H)-one (8a)

Prepared according to Scheme 3 via reductive amination of 7a.

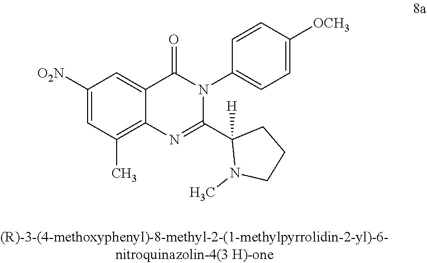 [SMILES: COc1ccc(-n2c([C@H]3CCCN3C)nc3c(C)cc([N+](=O)[O-])cc3c2=O)cc1]

### Synthesis of (R)-3-(4-methoxyphenyl)-8-methyl-2-(1-methylpyrrolidin-2-yl)-6-nitroquinazolin-4 (3H)-one (8a)

Acetic acid (0.02 mL) was added to the solution of 7a (76.10 mg, 0.20 mmol) and formaldehyde solution 37.0% in H₂O (32.43 mg, 0.40 mmol) in MeOH (2 mL) and CH₂Cl₂ (2 mL), stirred at room temperature for 1 h, then sodium cyanoborohydride (18.85 mg, 0.30 mmol) was added to the reaction mixture at 0° C., then stirred at room temperature overnight, then the solvent was removed in vacuo and the residue was diluted with CH₂Cl₂ (30 mL) and washed with saturated solutions of Na₂CO₃ (20 mL) and brine (20 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 8a (46.40 mg, 58.8%) as a pale yellow amorphous solid. ¹H NMR (500 MHz, CDCl₃) δ 8.95 (d, J=2.4 Hz, 1H), 8.39 (d, J=1.9 Hz, 1H), 7.19 (dd, J=8.5, 2.4 Hz, 1H), 7.12-7.04 (m, 3H), 3.90 (s, 3H), 3.47 (dd, J=8.0, 4.9 Hz, 1H), 3.30-3.23 (m, 1H), 2.73 (s, 3H), 2.50 (dd, J=16.0, 7.6 Hz, 1H), 2.34 (s, 3H), 2.09-2.00 (m, 2H), 1.98-1.94 (m Hz, 1H), 1.83-1.75 (m, 1H). MS (ESI) m/z: 395.1 (M+1)⁺.

### Example 58. Synthesis of (S)-3-(4-methoxyphenyl)-8-methyl-2-(1-methylpyrrolidin-2-yl)-6-nitroquinazolin-4 (3H)-one (8b)

Prepared according to Scheme 3 via reductive amination of 7b.

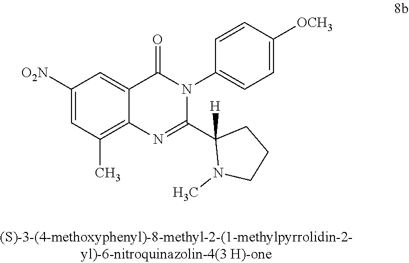 [SMILES: COc1ccc(-n2c([C@@H]3CCCN3C)nc3c(C)cc([N+](=O)[O-])cc3c2=O)cc1]

### Synthesis of (S)-3-(4-methoxyphenyl)-8-methyl-2-(1-methylpyrrolidin-2-yl)-6-nitroquinazolin-4 (3H)-one (8b)

Acetic acid (0.02 mL) was added to the solution of 7b (76.10 mg, 0.20 mmol) and formaldehyde solution 37.0% in H₂O (32.46 mg, 0.40 mmol) in MeOH (2 mL) and CH₂Cl₂ (2 mL), stirred at room temperature for 1 h, then sodium cyanoborohydride (18.85 mg, 0.30 mmol) was added to the reaction mixture at 0° C., then stirred at room temperature overnight, then the solvent was removed in vacuo and the residue was diluted with CH₂Cl₂ (30 mL) and washed with saturated solutions of Na₂CO₃ (20 mL) and brine (20 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 8b (45.60 mg, 57.8%) as a pale yellow amorphous solid. ¹H NMR (500 MHz, CDCl₃) δ 8.95 (d, J=2.5 Hz, 1H), 8.40 (d, J=1.9 Hz, 1H), 7.19 (dd, J=8.5, 2.5 Hz, 1H), 7.12-7.06 (m, 3H), 3.90 (s, 3H), 3.47 (dd, J=8.0, 4.9 Hz, 1H), 3.29-3.21 (m, 1H), 2.73 (s, 3H), 2.50 (dd, J=16.0, 7.5 Hz, 1H), 2.34 (s, 3H), 2.03 (dt, J=9.7, 7.9 Hz, 2H), 1.98-1.94 (m, 1H), 1.85-1.75 (m, 1H). MS (ESI) m/z: 395.1 (M+1)⁺.

### Example 59. Synthesis of (R)-3-(3-fluoro-4-methoxyphenyl)-8-methyl-6-nitro-2-pyrrolidin-2-yl) quinazolin-4 (3H)-one (7c)

Prepared according to Scheme 3.

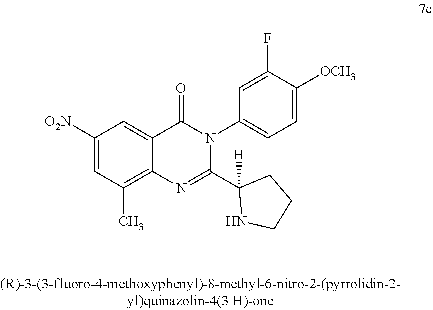 [SMILES: COc1ccc(-n2c([C@H]3CCCN3)nc3c(C)cc([N+](=O)[O-])cc3c2=O)cc1F]

Step 1. Synthesis of tert-butyl (R)-2-(3-(3-fluoro-4-methoxyphenyl)-8-methyl-6-nitro-4-oxo-3,4-dihydroquinazolin-2-yl) pyrrolidine-1-carboxylate (6c). 2-amino-3-methyl-5-nitrobenzoic acid (98.08 mg, 0.50 mmol) and Boc-D-proline (140.00 mg, 0.65 mmol) were dissolved in dry pyridine (3 mL). Triphenyl phosphite (0.40 mL, 1.50 mmol) was added to the mixture and the reaction mixture was heated at 100° C. with MWI for 40 min. 3-Fluoro-4-methoxyaniline (84.68 mg, 0.60 mmol) was added to the mixture and the reaction mixture was heated at 100° C. with MWI for 20 min. Pyridine was removed in vacuo and the residue was diluted with CH₂Cl₂ (60 mL) and washed with 1N HCl aqueous (20 mL). The organic layer was washed with H₂O (30 mL) and brine (30 mL), dried over anhydrous Na₂SO₄, and filtered, then the solvent was evaporated under reduced pressure. The residue was purified by flash chromatography (0% Ethyl acetate/CH₂Cl₂˜5% Ethyl acetate/CH₂Cl₂) to afford 6c (82.20 mg, 33.0%) as pale yellow solid. ¹H NMR (500 MHz, CDCl₃) δ 8.89 (dd, J=16.5, 2.1 Hz, 1H), 8.36 (d, J=23.5 Hz, 1H), 7.40-7.08 (m, 2H), 7.11-6.96 (m, 1H), 4.67-4.48 (m, 1H), 3.98 (dd, J=11.8, 3.1 Hz, 3H), 3.82-3.63 (m, 1H), 3.58-3.51 (m, 1H), 2.63 (d, J=4.0 Hz, 3H), 2.19-1.82 (m, 4H), 1.43 (d, J=5.4 Hz, 5H), 1.27 (d, J=2.1 Hz, 4H). MS (ESI) m/z: 499.1 (M+1)⁺.

Step 2. Synthesis of (R)-3-(3-fluoro-4-methoxyphenyl)-8-methyl-6-nitro-2-(pyrrolidin-2-yl) quinazolin-4 (3H)-one (7c)

Under nitrogen, trifluoroacetic acid (0.18 mL, 2.40 mmol) was added to a solution of 6c (60.00 mg, 0.12 mmol) in dry CH₂Cl₂ (2 mL) at 0° C., then stirred at room temperature overnight, then the solvent was removed in vacuo and the residue was diluted with CH₂Cl₂ (20 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 7c (26.00 mg, 54.4%) as pale yellow solid. ¹H NMR (500 MHz, CDCl₃) δ 8.93 (d, J=2.2 Hz, 1H), 8.40 (d, J=1.7 Hz, 1H), 7.17-6.98 (m, 3H), 3.98 (s, 3H), 3.92 (dd, J=14.6, 7.0 Hz, 1H), 3.29 (dt, J=11.4, 5.9 Hz, 1H), 2.84 (td, J=11.0, 6.5 Hz, 1H), 2.30 (s, 2H), 2.71 (s, 3H), 1.88-1.75 (m, 4H). MS (ESI) m/z: 399.1 (M+1)⁺.

### Example 60. Synthesis of (R)-3-(4-chloro-3-fluorophenyl)-8-methyl-6-nitro-2-(pyrrolidin-2-yl) quinazolin-4 (3H)-one (7d)

Prepared according to Scheme 3.

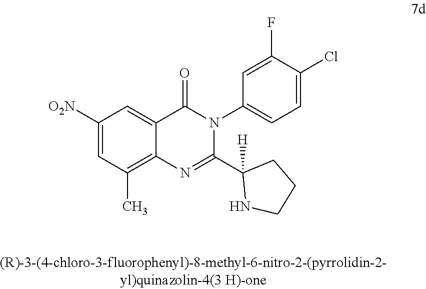 [SMILES: Cc1cc([N+](=O)[O-])cc2c(=O)n(-c3ccc(Cl)c(F)c3)c([C@H]3CCCN3)nc12]

Step 1. Synthesis of tert-butyl (R)-2-(3-(4-chloro-3-fluorophenyl)-8-methyl-6-nitro-4-oxo-3,4-dihydroquinazo-lin-2-yl) pyrrolidine-1-carboxylate (6d). 2-amino-3-methyl-5-nitrobenzoic acid (78.65 mg, 0.40 mmol) and Boc-D-proline (112.00 mg, 0.32 mmol) were dissolved in dry pyridine (3 mL). Triphenyl phosphite (0.32 mL, 1.20 mmol) was added to the mixture and the reaction mixture was heated at 100° C. with MWI for 40 min. 4-Chloro-3-fluoroaniline (69.86 mg, 0.48 mmol) was added to the mixture and the reaction mixture was heated at 100° C. with MWI for 30 min. Pyridine was removed in vacuo and the residue was diluted with CH₂Cl₂ (60 mL) and washed with 1N HCl aqueous (20 mL). The organic layer was washed with H₂O (30 mL) and brine (30 mL), dried over anhydrous Na₂SO₄, and filtered, then the solvent was evaporated under reduced pressure. The residue was purified by flash chromatography (0% Ethyl acetate/Hexane˜40% Ethyl acetate/Hexane) to afford 6d (38.80 mg, 19.3%) as pale yellow solid. ¹H NMR (400 MHz, CDCl₃) δ 8.88 (d, J=11.3 Hz, 1H), 8.38 (d, J=18.1 Hz, 1H), 7.69-7.54 (m, 1H), 7.51-7.01 (m, 2H), 4.58-4.45 (m, 1H), 3.86-3.69 (m, 1H), 3.61-3.45 (m, 1H), 2.65 (d, J=3.4 Hz, 3H), 2.20-1.86 (m, 4H), 1.42 (d, J=1.9 Hz, 5H), 1.27 (d, J=4.8 Hz, 4H). MS (ESI) m/z: 503.1 (M+1)⁺.

Step 2. Synthesis of (R)-3-(4-chloro-3-fluorophenyl)-8-methyl-6-nitro-2-(pyrrolidin-2-yl) quinazolin-4 (3H)-one (7d)

Under nitrogen, trifluoroacetic acid (0.088 mL, 1.20 mmol) was added to a solution of 6d (30.00 mg, 0.06 mmol) in dry CH₂Cl₂ (2 mL) at 0° C., then stirred at room temperature overnight, then the solvent was removed in vacuo and the residue was diluted with CH₂Cl₂ (20 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 7d (19.68 mg, 81.4%) as pale yellow solid. ¹H NMR (400 MHz, CDCl₃) δ 8.91 (d, J=2.3 Hz, 1H), 8.41 (d, J=1.7 Hz, 1H), 7.63 (t, J=8.1 Hz, 1H), 7.21-7.05 (m, 2H), 3.89 (t, J=6.8 Hz, 1H), 3.31-3.25 (m, 1H), 2.89-2.83 (m, 1H), 2.71 (d, J=8.6 Hz, 3H), 1.91-1.76 (m, 4H). MS (ESI) m/z: 403.1 (M+1)⁺.

### Example 61. Synthesis of 2-((2R,4S)-4-fluoropyrrolidin-2-yl)-3-(4-methoxyphenyl)-8-methyl-6-nitroquinazolin-4 (3H)-one (7e)

Prepared according to Scheme 3.

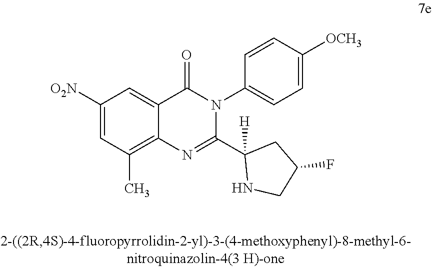 [SMILES: COc1ccc(-n2c([C@H]3C[C@H](F)CN3)nc3c(C)cc([N+](=O)[O-])cc3c2=O)cc1]

Step 1. Synthesis of tert-butyl (2R,4S)-4-fluoro-2-(3-(4-methoxyphenyl)-8-methyl-6-nitro-4-oxo-3,4-dihydro-quinazolin-2-yl) pyrrolidine-1-carboxylate (6e). 2-amino-3-methyl-5-nitrobenzoic acid (49.04 mg, 0.25 mmol) and (2R,4S)-1-(tert-butoxycarbonyl)-4-fluoropyrrolidine-2-carboxylic acid (75.80 mg, 0.33 mmol) were dissolved in dry pyridine (1.5 mL). Triphenyl phosphite (0.20 mL, 0.75 mmol) was added to the mixture and the reaction mixture was heated at 100° C. with MWI for 40 min. 4-methoxyaniline (36.95 mg, 0.30 mmol) was added to the mixture and the reaction mixture was heated at 100° C. with MWI for 1×20 min. Pyridine was removed in vacuo and the residue was diluted with CH₂Cl₂ (30 mL) and washed with 1N HCl aqueous (20 mL). The organic layer was washed with H₂O (20 mL) and brine (20 mL), dried over anhydrous Na₂SO₄, and filtered, then the solvent was evaporated under reduced pressure. The residue was purified by flash chromatography (0% Ethyl acetate/Hexane˜25% Ethyl acetate/Hexane) to afford 6e (49.30 mg, 39.6%) as pale yellow solid. ¹H NMR (500 MHz, CDCl₃) δ 8.90 (dd, J=22.3, 2.3 Hz, 1H), 8.35 (dd, J=27.0, 1.7 Hz, 1H), 7.51-7.21 (m, 1H), 7.15-7.02 (m, 3H), 5.25 (dd, J=52.7, 16.8 Hz, 1H), 4.72 (dt, J=28.2, 7.7 Hz, 1H), 4.04-3.73 (m, 5H), 2.60 (d, J=6.2 Hz, 3H), 2.45-2.17 (m, 2H), 1.42 (s, 5H), 1.24 (s, 4H). MS (ESI) m/z: 499.1 (M+1)⁺.

Step 2. Synthesis of 2-((2R,4S)-4-fluoropyrrolidin-2-yl)-3-(4-methoxyphenyl)-8-methyl-6-nitroquinazolin-4 (3H)-one (7e)

Under nitrogen, trifluoroacetic acid (0.13 mL, 1.80 mmol) was added to a solution of 6e (45.00 mg, 0.09 mmol) in dry CH₂Cl₂ (1.5 mL) at 0° C., then stirred at room temperature overnight, then the solvent was removed in vacuo and the residue was diluted with CH₂Cl₂ (20 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 7e (30.70 mg, 85.6%) as pale yellow solid: mp 183.06-185.34° C. ¹H NMR (500 MHz, CDCl₃) δ 8.93 (d, J=2.4 Hz, 1H), 8.38 (d, J=1.9 Hz, 1H), 7.28 (dd, J=7.1, 3.9 Hz, 1H), 7.15 (dd, J=9.0, 2.4 Hz, 1H), 7.12-7.06 (m, 2H), 5.31 (dt, J=54.4, 3.9 Hz, 1H), 4.25 (t, J=7.7 Hz, 1H), 3.90 (s, 3H), 3.42-3.31 (m, 1H), 3.25-3.16 (m, 1H), 2.67 (s, 3H), 2.41-2.30 (m, 1H), 2.21-2.15 (m, 1H). MS (ESI) m/z: 399.1 (M+1)⁺.

### Example 62. Synthesis of 2-((2R,4S)-4-fluoropyrrolidin-2-yl)-3-(3-fluoro-4-methoxyphenyl)-8-methyl-6-nitroquinazolin-4 (3H)-one (7f)

Prepared according to Scheme 3.

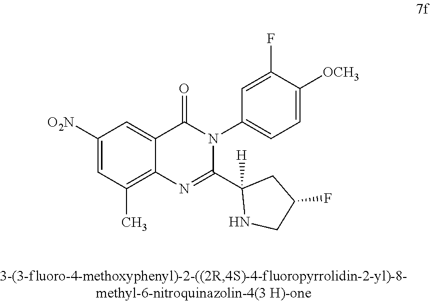 [SMILES: COc1ccc(-n2c([C@H]3C[C@H](F)CN3)nc3c(C)cc([N+](=O)[O-])cc3c2=O)cc1F]

Step 1. Synthesis of tert-butyl (2R,4S)-4-fluoro-2-(3-(3-fluoro-4-methoxyphenyl)-8-methyl-6-nitro-4-oxo-3,4-dihydroquinazolin-2-yl) pyrrolidine-1-carboxylate (6f). 2-amino-3-methyl-5-nitrobenzoic acid (78.65 mg, 0.40 mmol) and (2R,4S)-1-(tert-butoxycarbonyl)-4-fluoropyrrolidine-2-carboxylic acid (121.28 mg, 0.52 mmol) were dissolved in dry pyridine (3 mL). Triphenyl phosphite (0.31 mL, 1.20 mmol) was added to the mixture and the reaction mixture was heated at 100° C. with MWI for 40 min. 3-Fluoro-4-methoxyaniline (67.75 mg, 0.48 mmol) was added to the mixture and the reaction mixture was heated at 100° C. with MWI for 1×20 min. Pyridine was removed in vacuo and the residue was diluted with CH₂Cl₂ (40 mL) and washed with 1N HCl aqueous (20 mL). The organic layer was washed with H₂O (20 mL) and brine (20 mL), dried over anhydrous Na₂SO₄, and filtered, then the solvent was evaporated under reduced pressure. The residue was purified by flash chromatography (0% Ethyl acetate/Hexane to 40% Ethyl acetate/Hexane) to afford 6f (70.80 mg, 34.3%) as pale yellow solid. ¹H NMR (500 MHz, CDCl₃) δ 8.78 (d, J=23.0 Hz, 1H), 8.26 (d, J=28.5 Hz, 1H), 7.36-7.08 (m, 1H), 7.09-7.00 (m, 1H), 6.95-6.87 (m, 1H), 5.20 (dd, J=52.6, 18.6 Hz, 1H), 4.72-4.52 (m, 1H), 3.91 (dd, J=10.9, 5.2 Hz, 3H), 3.85-3.70 (m, 2H), 2.52 (d, J=7.9 Hz, 3H), 2.38-2.16 (m, 2H), 1.34 (d, J=6.0 Hz, 5H), 1.17 (s, 4H). MS (ESI) m/z: 517.1 (M+1)⁺.

Step 2. Synthesis of 2-((2R,4S)-4-fluoropyrrolidin-2-yl)-3-(3-fluoro-4-methoxyphenyl)-8-methyl-6-nitroquin-azolin-4 (3H)-one (7f)

Under nitrogen, trifluoroacetic acid (0.18 mL, 2.40 mmol) was added to a solution of 6f (60.00 mg, 0.12 mmol) in dry CH₂Cl₂ (1.5 mL) at 0° C., then stirred at room temperature overnight, then the solvent was removed in vacuo and the residue was diluted with CH₂Cl₂ (20 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 7f (23.70 mg, 47.4%) as pale yellow solid. ¹H NMR (400 MHz, CDCl₃) δ 8.85 (d, J=2.5 Hz, 1H), 8.33 (d, J=2.3 Hz, 1H), 7.13-7.02 (m, 2H), 7.06-6.91 (m, 1H), 5.26 (dt, J=54.4, 3.9 Hz, 1H), 4.18 (dd, J=13.3, 7.4 Hz, 1H), 3.92 (s, 3H), 3.35-3.22 (m, 1H), 3.18-3.11 (m, 1H), 2.60 (s, 3H), 2.41-2.32 (m, 1H), 2.19-2.05 (m, 1H). MS (ESI) m/z: 417.1 (M+1)⁺.

### Example 63. Synthesis of 2-((2S,4R)-4-fluoropyrrolidin-2-yl)-3-(4-methoxyphenyl)-8-methyl-6-nitroquinazolin-4 (3H)-one (7g)

Prepared according to Scheme 3.

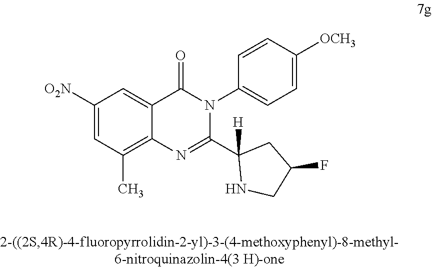 [SMILES: COc1ccc(-n2c([C@@H]3C[C@@H](F)CN3)nc3c(C)cc([N+](=O)[O-])cc3c2=O)cc1]

Step 1. Synthesis of tert-butyl (2S,4R)-4-fluoro-2-(3-(4-methoxyphenyl)-8-methyl-6-nitro-4-oxo-3,4-dihydroquinazolin-2-yl) pyrrolidine-1-carboxylate (6g). 2-amino-3-methyl-5-nitrobenzoic acid (78.48 mg, 0.40 mmol) and (2S,4R)-1-(tert-butoxycarbonyl)-4-fluoropyrrolidine-2-carboxylic acid (121.28 mg, 0.52 mmol) were dissolved in dry pyridine (2.5 mL). Triphenyl phosphite (0.34 mL, 1.20 mmol) was added to the mixture and the reaction mixture was heated at 100° C. with MWI for 40 min. 4-methoxyaniline (59.16 mg, 0.48 mmol) was added to the mixture and the reaction mixture was heated at 100° C. with MWI for 1×20 min. Pyridine was removed in vacuo and the residue was diluted with CH₂Cl₂ (30 mL) and washed with 1N HCl aqueous (20 mL). The organic layer was washed with H₂O (20 mL) and brine (20 mL), dried over anhydrous Na₂SO₄, and filtered, then the solvent was evaporated under reduced pressure. The residue was purified by flash chromatography (0% Ethyl acetate/Hexane to 40% Ethyl acetate/Hexane) to afford 6g (66.80 mg, 33.5%) as pale yellow solid. ¹H NMR (500 MHz, CDCl₃) δ 8.89 (dd, J=22.0, 2.5 Hz, 1H), 8.42-8.30 (m, 1H), 7.51-7.18 (m, 1H), 7.14-7.01 (m, 3H), 5.37-5.10 (m, 1H), 4.82-4.63 (m, 1H), 4.05-3.75 (m, 5H), 2.60 (d, J=6.6 Hz, 3H), 2.44-2.17 (m, 2H), 1.42 (s, 5H), 1.24 (s, 4H). MS (ESI) m/z: 499.1 (M+1)⁺.

Step 2. Synthesis of 2-((2S,4R)-4-fluoropyrrolidin-2-yl)-3-(4-methoxyphenyl)-8-methyl-6-nitroquinazolin-4 (3H)-one (7g)

Under nitrogen, trifluoroacetic acid (0.15 mL, 2.00 mmol) was added to a solution of 6g (50.00 mg, 0.10 mmol) in dry CH₂Cl₂ (2 mL) at 0° C., then stirred at room temperature overnight, then the solvent was removed in vacuo and the residue was diluted with CH₂Cl₂ (20 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂ to 5% MeOH/CH₂Cl₂) to afford 7g (35.40 mg, 88.1%) as pale yellow solid. ¹H NMR (400 MHz, CDCl₃) δ 8.93 (d, J=2.2 Hz, 1H), 8.38 (d, J=1.6 Hz, 1H), 7.29 (d, J=3.1 Hz, 1H), 7.15 (dd, J=9.0, 2.3 Hz, 1H), 7.11-7.04 (m, 2H), 5.31 (d, J=54.4 Hz, 1H), 4.25 (t, J=7.7 Hz, 1H), 3.90 (s, 3H), 3.41-3.32 (m, 1H), 3.25-3.11 (m, 1H), 2.67 (s, 3H), 2.41-2.34 (m 1H), 2.25-2.10 (m, 1H). MS (ESI) m/z: 399.1 (M+1)⁺.

### Example 64. Synthesis of 2-((2S,4R)-4-fluoropyrrolidin-2-yl)-3-(3-fluoro-4-methoxyphenyl)-8-methyl-6-nitroquin-azolin-4 (3H)-one (7h)

Prepared according to Scheme 3.

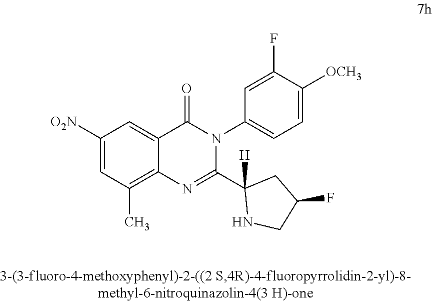 [SMILES: COc1ccc(-n2c([C@@H]3C[C@@H](F)CN3)nc3c(C)cc([N+](=O)[O-])cc3c2=O)cc1F]

Step 1. Synthesis of tert-butyl (2S,4R)-4-fluoro-2-(3-(3-fluoro-4-methoxyphenyl)-8-methyl-6-nitro-4-oxo-3,4-dihydroquinazolin-2-yl) pyrrolidine-1-carboxylate (6h). 2-amino-3-methyl-5-nitrobenzoic acid (78.65 mg, 0.40 mmol) and (2S,4R)-1-(tert-butoxycarbonyl)-4-fluoropyrrolidine-2-carboxylic acid (121.28 mg, 0.52 mmol) were dissolved in dry pyridine (2.5 mL). Triphenyl phosphite (0.31 mL, 1.20 mmol) was added to the mixture and the reaction mixture was heated at 100° C. with MWI for 40 min. 3-Fluoro-4-methoxyaniline (67.75 mg, 0.48 mmol) was added to the mixture and the reaction mixture was heated at 100° C. with MWI for 1×20 min. Pyridine was removed in vacuo and the residue was diluted with CH₂Cl₂ (40 mL) and washed with 1N aqueous HCl (20 mL). The organic layer was washed with H₂O (20 mL) and brine (20 mL), dried over anhydrous Na₂SO₄, and filtered, then the solvent was evaporated under reduced pressure. The residue was purified by flash chromatography (0% Ethyl acetate/Hexane 40% Ethyl acetate/Hexane) to afford 6h (65.60 mg, 35.8%) as pale yellow solid. ¹H NMR (500 MHz, CDCl₃) δ 8.90 (dd, J=19.6, 2.1 Hz, 1H), 8.43-8.32 (m, 1H), 7.38-7.11 (m, 2H), 7.08-6.91 (m, 1H), 5.41-5.16 (m, 1H), 4.79-4.61 (m, 1H), 4.04-3.76 (m, 5H), 2.61 (d, J=6.1 Hz, 3H), 2.45-2.17 (m, 2H), 1.41 (t, J=5.6 Hz, 5H), 1.24 (d, J=1.9 Hz, 4H). MS (ESI) m/z: 517.1 (M+1)⁺.

Step 2. Synthesis of 2-((2S,4R)-4-fluoropyrrolidin-2-yl)-3-(3-fluoro-4-methoxyphenyl)-8-methyl-6-nitroquin-azolin-4 (3H)-one (7h)

Under nitrogen, trifluoroacetic acid (0.15 mL, 2.00 mmol) was added to a solution of 6h (50.00 mg, 0.10 mmol) in dry CH₂Cl₂ (2 mL) at 0° C., then stirred at room temperature overnight, then the solvent was removed in vacuo and the residue was diluted with CH₂Cl₂ (20 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂ to 5% MeOH/CH₂Cl₂) to afford 7h (36.20 mg, 86.9%) as pale yellow solid. ¹H NMR (400 MHz, CDCl₃) δ 8.91 (d, J=1.7 Hz, 1H), 8.39 (s, 1H), 7.20-7.10 (m, 2H), 7.05-6.93 (m, 1H), 5.34 (d, J=54.4 Hz, 1H), 4.25 (dd, J=14.3, 7.2 Hz, 1H), 3.99 (s, 3H), 3.45-3.29 (m, 1H), 3.24-3.18 (m, 1H), 2.67 (s, 3H), 2.56-2.31 (m, 1H), 2.27-2.10 (m, 1H). MS (ESI) m/z: 417.1 (M+1)⁺.

### Example 65. Synthesis of (S)-3-(3-fluoro-4-methoxyphenyl)-8-methyl-6-nitro-2-(pyrrolidin-3-yl) quinazolin-4 (3H)-one (7i)

Prepared according to Scheme 3.

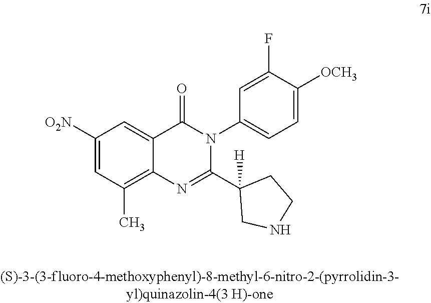 [SMILES: COc1ccc(-n2c([C@H]3CCNC3)nc3c(C)cc([N+](=O)[O-])cc3c2=O)cc1F]

Step 1. Synthesis of tert-butyl (S)-3-(3-(3-fluoro-4-methoxyphenyl)-8-methyl-6-nitro-4-oxo-3,4-dihydroquin-azolin-2-yl) pyrrolidine-1-carboxylate (6i). 2-amino-3-methyl-5-nitrobenzoic acid (98.08 mg, 0.50 mmol) and (S)—N-Boc-pyrrolidine-3-carboxylic acid (140.00 mg, 0.65 mmol) were dissolved in dry pyridine (3 mL). Triphenyl phosphite (0.40 mL, 1.50 mmol) was added to the mixture and the reaction mixture was heated at 110° C. with MWI for 40 min. 3-Fluoro-4-methoxyaniline (84.68 mg, 0.60 mmol) was added to the mixture and the reaction mixture was heated at 100° C. with MWI for 20 min. Pyridine was removed in vacuo and the residue was diluted with CH₂Cl₂ (80 mL) and washed with 1N HCl aqueous (40 mL). The organic layer was washed with H₂O (30 mL) and brine (30 mL), dried over anhydrous Na₂SO₄, and filtered, then the solvent was evaporated under reduced pressure. The residue was purified by flash chromatography (0% Ethyl acetate/Hexane˜50% acetate/Hexane) to afford 6i (37.90 mg, 15.1%) as pale yellow solid. ¹H NMR (400 MHz, CDCl₃) δ 8.89 (s, 1H), 8.38 (s, 1H), 7.18-6.96 (m, 3H), 3.99 (s, 3H), 3.81-3.48 (m, 3H), 3.38-3.12 (m, 2H), 2.67 (s, 3H), 2.45-2.28 (m, 1H), 2.11-1.98 (m, 1H), 1.46 (s, 9H). MS (ESI) m/z: 499.1 (M+1)⁺.

Step 2. Synthesis of (S)-3-(3-fluoro-4-methoxyphenyl)-8-methyl-6-nitro-2-(pyrrolidin-3-yl) quinazolin-4 (3H)-one (7i) Under nitrogen, trifluoroacetic acid (0.10 mL, 1.40 mmol) was added to a solution of 6i (35.00 mg, 0.07 mmol) in dry CH₂Cl₂ (1.5 mL) at 0° C., then stirred at room temperature overnight, then the solvent was removed in vacuo and the residue was diluted with CH₂Cl₂ (20 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 7i (15.40 mg, 55.1%) as pale yellow amorphous solid. ¹H NMR (500 MHz, CDCl₃) δ 8.93 (d, J=2.5 Hz, 1H), 8.39 (d, J=1.9 Hz, 1H), 7.17-7.11 (m, 1H), 7.05-6.97 (m, 2H), 3.99 (s, 3H), 3.43-3.34 (m, 1H), 3.31-3.25 (m, 1H), 3.12-3.05 (m, 1H), 3.03-2.98 (m, 1H), 2.96-2.87 (m, 1H), 2.66 (s, 3H), 2.24-2.11 (m, 3H), 2.01-1.95 (m, 1H). MS (ESI) m/z: 399.1 (M+1)⁺.

### Example 66. Synthesis of (S)-3-(3-fluoro-4-methoxyphenyl)-8-methyl-2-(1-methylpyrrolidin-3-yl)-6-nitroquinazolin-4 (3H)-one (8c)

Prepared according to Scheme 3.

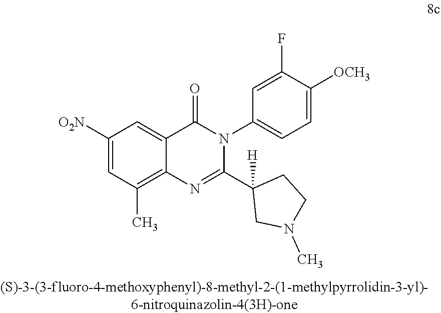 [SMILES: COc1ccc(-n2c([C@H]3CCN(C)C3)nc3c(C)cc([N+](=O)[O-])cc3c2=O)cc1F]

### Synthesis of (S)-3-(3-fluoro-4-methoxyphenyl)-8-methyl-2-(1-methylpyrrolidin-3-yl)-6-nitroquinazolin-4 (3H)-one (8c)

Acetic acid (0.01 mL) was added to the solution of 7i (14.00 mg, 0.035 mmol) and formaldehyde solution 37.0% in H₂O (5.68 mg, 0.070 mmol) in MeOH (0.6 mL) and CH₂Cl₂ (0.6 mL), stirred at room temperature for 1 h, then sodium cyanoborohydride (3.45 mg, 0.055 mmol) was added to the reaction mixture at 0° C., then stirred at room temperature overnight, then the solvent was removed in vacuo and the residue was diluted with CH₂Cl₂ (30 mL) and washed with saturated solutions of Na₂CO₃ (20 mL) and brine (20 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 8c (8.96 mg, 62.1%) as pale yellow solid. ¹H NMR (500 MHz, CDCl₃) δ 8.93 (d, J=2.5 Hz, 1H), 8.40 (d, J=2.0 Hz, 1H), 7.12 (t, J=8.7 Hz, 1H), 7.05-6.95 (m, 2H), 3.98 (s, 3H), 3.31-3.24 (m, 1H), 2.89 (t, J=8.5 Hz, 1H), 2.86-2.81 (m, 2H), 2.71 (s, 3H), 2.58-2.51 (m, 1H), 2.39 (s, 3H), 2.34-2.26 (m, 1H), 1.95-1.86 (m, 1H). MS (ESI) m/z: 413.1 (M+1)⁺.

### Example 67. Synthesis of (R)-3-(3-fluoro-4-methoxyphenyl)-8-methyl-6-nitro-2-(pyrrolidin-3-yl) quinazolin-4 (3H)-one (7j)

Prepared according to Scheme 3.

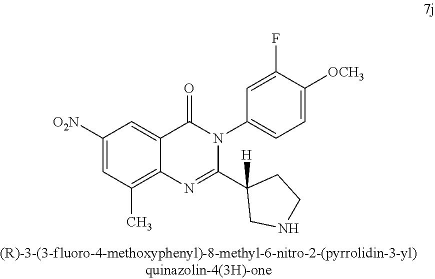 [SMILES: COc1ccc(-n2c([C@@H]3CCNC3)nc3c(C)cc([N+](=O)[O-])cc3c2=O)cc1F]

Step 1. Synthesis of tert-butyl (R)-3-(3-(3-fluoro-4-methoxyphenyl)-8-methyl-6-nitro-4-oxo-3,4-dihydroquinazolin-2-yl) pyrrolidine-1-carboxylate (6j). 2-amino-3-methyl-5-nitrobenzoic acid (98.08 mg, 0.50 mmol) and (R)—N-Boc-pyrrolidine-3-carboxylic acid (140.00 mg, 0.65 mmol) were dissolved in dry pyridine (3 mL). Triphenyl phosphite (0.40 mL, 1.50 mmol) was added to the mixture and the reaction mixture was heated at 110° C. with MWI for 40 min. 3-Fluoro-4-methoxyaniline (84.68 mg, 0.60 mmol) was added to the mixture and the reaction mixture was heated at 100° C. with MWI for 20 min. Pyridine was removed in vacuo and the residue was diluted with CH₂Cl₂ (100 mL) and washed with 1N HCl aqueous (40 mL). The organic layer was washed with H₂O (30 mL) and brine (30 mL), dried over anhydrous Na₂SO₄, and filtered, then the solvent was evaporated under reduced pressure. The residue was purified by flash chromatography (0% Ethyl acetate/Hexane˜50% acetate/Hexane) to afford 6j (37.80 mg, 15.1%) as pale yellow solid. ¹H NMR (400 MHz, CDCl₃) δ 8.88 (s, 1H), 8.36 (s, 1H), 7.21-6.96 (m, 3H), 3.98 (s, 3H), 3.85-3.48 (m, 3H), 3.42-3.11 (m, 2H), 2.65 (s, 3H), 2.45-2.28 (m, 1H), 2.15-1.98 (m, 1H), 1.45 (s, 9H). MS (ESI) m/z: 499.1 (M+1)⁺.

Step 2. Synthesis of (R)-3-(3-fluoro-4-methoxyphenyl)-8-methyl-6-nitro-2-(pyrrolidin-3-yl) quinazolin-4 (3H)-one (7j) Under nitrogen, trifluoroacetic acid (0.10 mL, 1.40 mmol) was added to a solution of 6j (35.00 mg, 0.07 mmol) in dry CH₂Cl₂ (1.5 mL) at 0° C., then stirred at room temperature overnight, then the solvent was removed in vacuo and the residue was diluted with CH₂Cl₂ (20 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 7j (18.80 mg, 67.4%) as pale yellow amorphous solid. ¹H NMR (500 MHz, CDCl₃) δ 8.91 (d, J=2.5 Hz, 1H), 8.41 (d, J=1.8 Hz, 1H), 7.18-7.11 (m, 1H), 7.08-6.98 (m, 2H), 3.98 (s, 3H), 3.45-3.34 (m, 1H), 3.34-3.25 (m, 1H), 3.15-3.04 (m, 1H), 3.06-2.98 (m, 1H), 2.96-2.85 (m, 1H), 2.65 (s, 3H), 2.25-2.11 (m, 3H), 2.06-1.95 (m, 1H). MS (ESI) m/z: 399.1 (M+1)⁺.

### Example 68. Synthesis of (R)-3-(3-fluoro-4-methoxyphenyl)-8-methyl-2-(1-methylpyrrolidin-3-yl)-6-nitroquinazolin-4 (3H)-one (8d)

Prepared according to Scheme 3.

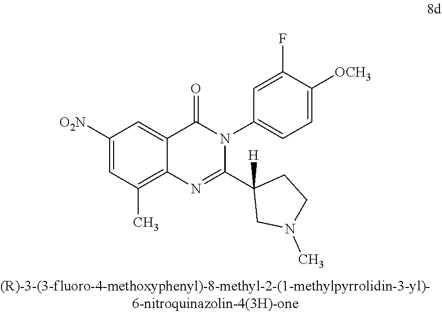 [SMILES: COc1ccc(-n2c([C@@H]3CCN(C)C3)nc3c(C)cc([N+](=O)[O-])cc3c2=O)cc1F]

### Synthesis of (R)-3-(3-fluoro-4-methoxyphenyl)-8-methyl-2-(1-methylpyrrolidin-3-yl)-6-nitroquinazolin-4 (3H)-one (8d)

Acetic acid (0.01 mL) was added to the solution of 7j (15.00 mg, 0.038 mmol) and formaldehyde solution 37.0% in H₂O (6.18 mg, 0.076 mmol) in MeOH (0.6 mL) and CH₂Cl₂ (0.6 mL), stirred at room temperature for 1 h, then sodium cyanoborohydride (3.55 mg, 0.056 mmol) was added to the reaction mixture at 0° C., then stirred at room temperature overnight, then the solvent was removed in vacuo and the residue was diluted with CH₂Cl₂ (30 mL) and washed with saturated solutions of Na₂CO₃ (20 mL) and brine (20 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 8d (9.86 mg, 62.9%) as pale yellow solid. ¹H NMR (500 MHz, CDCl₃) δ 8.93 (d, J=2.6 Hz, 1H), 8.40 (d, J=1.9 Hz, 1H), 7.12 (t, J=8.7 Hz, 1H), 7.05-6.96 (m, 2H), 3.98 (s, 3H), 3.31-3.24 (m, 1H), 2.91-2.86 (m, 1H), 2.85-2.78 (m, 1H), 2.71 (s, 3H), 2.61-2.55 (m, 1H), 2.39 (s, 3H), 2.34-2.26 (m, 2H), 1.96-1.88 m, 1H). MS (ESI) m/z: 413.1 (M+1)⁺.

### Example 69. Synthesis of (S)-3-(3-fluoro-4-methoxyphenyl)-8-methyl-6-nitro-2-(pyrrolidin-2-yl) quinazolin-4 (3H)-one (7k)

Prepared according to Scheme 3.

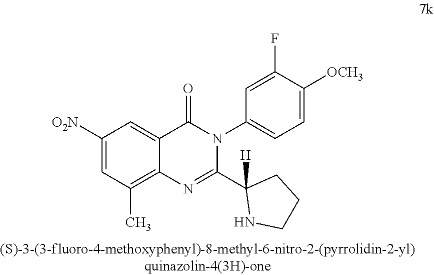 [SMILES: COc1ccc(-n2c([C@@H]3CCCN3)nc3c(C)cc([N+](=O)[O-])cc3c2=O)cc1F]

Step 1. Synthesis of tert-butyl (S)-2-(3-(3-fluoro-4-methoxyphenyl)-8-methyl-6-nitro-4-oxo-3,4-dihydroquinazolin-2-yl) pyrrolidine-1-carboxylate (6k). 2-amino-3-methyl-5-nitrobenzoic acid (196.60 mg, 1.00 mmol) and Boc-L-proline (280.00 mg, 1.30 mmol) were dissolved in dry pyridine (5 mL). Triphenyl phosphite (0.79 mL, 3.00 mmol) was added to the mixture and the reaction mixture was heated at 100° C. with MWI for 40 min. 3-Fluoro-4-methoxyaniline (169.40 mg, 1.20 mmol) was added to the mixture and the reaction mixture was heated at 100° C. with MWI for 30 min. Pyridine was removed in vacuo and the residue was diluted with CH₂Cl₂ (100 mL) and washed with 1N HCl aqueous (40 mL). The organic layer was washed with H₂O (60 mL) and brine (60 mL), dried over anhydrous Na₂SO₄, and filtered, then the solvent was evaporated under reduced pressure. The residue was purified by flash chromatography (0% Ethyl acetate/CH₂Cl₂˜5% Ethyl acetate/CH₂Cl₂) to afford 6k (0.18 g, 37.8%) as pale yellow solid. ¹H NMR (500 MHz, CDCl₃) δ 8.86 (dd, J=16.6, 2.1 Hz, 1H), 8.35 (d, J=23.6 Hz, 1H), 7.41-7.06 (m, 2H), 7.11-6.95 (m, 1H), 4.68-4.51 (m, 1H), 3.96 (dd, J=11.6, 3.1 Hz, 3H), 3.824-3.61 (m, 1H), 3.58-3.51 (m, 1H), 2.65 (d, J=4.0 Hz, 3H), 2.21-1.82 (m, 4H), 1.43 (d, J=5.4 Hz, 5H), 1.28 (d, J=2.1 Hz, 4H). MS (ESI) m/z: 499.1 (M+1)⁺.

Step 2. Synthesis of (S)-3-(3-fluoro-4-methoxyphenyl)-8-methyl-6-nitro-2-(pyrrolidin-2-yl) quinazolin-4 (3H)-one (7k)

Under nitrogen, trifluoroacetic acid (0.54 mL, 7.20 mmol) was added to a solution of 6k (0.18 g, 0.36 mmol) in dry CH₂Cl₂ (6 mL) at 0° C., then stirred at room temperature overnight, then the solvent was removed in vacuo and the residue was diluted with CH₂Cl₂ (40 mL) and washed with saturated solutions of Na₂CO₃ (30 mL) and brine (30 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 7k (0.10 g, 69.7%) as pale yellow solid. ¹H NMR (500 MHz, CDCl₃) δ 8.91 (d, J=2.1 Hz, 1H), 8.41 (d, J=1.8 Hz, 1H), 7.18-6.96 (m, 3H), 3.98 (s, 3H), 3.91 (dd, J=14.8, 7.1 Hz, 1H), 3.28 (dt, J=11.5, 6.1 Hz, 1H), 2.85 (td, J=11.0, 6.5 Hz, 1H), 2.31 (s, 2H), 2.71 (s, 3H), 1.88-1.71 (m, 4H). MS (ESI) m/z: 399.1 (M+1)⁺.

### Example 70. Synthesis of (S)-3-(3-fluoro-4-methoxyphenyl)-8-methyl-2-(1-methylpyrrolidin-2-yl)-6-nitroquinazolin-4 (3H)-one (8e)

Prepared according to Scheme 3.

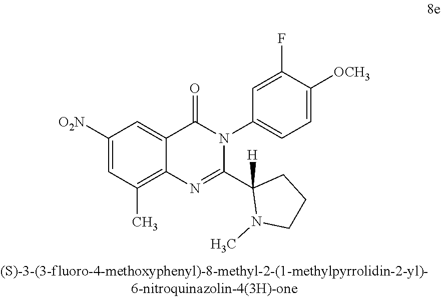 [SMILES: COc1ccc(-n2c([C@@H]3CCCN3C)nc3c(C)cc([N+](=O)[O-])cc3c2=O)cc1F]

### Synthesis of (S)-3-(3-fluoro-4-methoxyphenyl)-8-methyl-2-(1-methylpyrrolidin-2-yl)-6-nitroquinazol-in-4 (3H)-one (8e)

Acetic acid (0.02 mL) was added to the solution of 7k (39.85 mg, 0.10 mmol) and formaldehyde solution 37.0% in H₂O (16.25 mg, 0.20 mmol) in MeOH (1.5 mL) and CH₂Cl₂ (1.5 mL), stirred at room temperature for 1 h, then sodium cyanoborohydride (9.45 mg, 0.15 mmol) was added to the reaction mixture at 0° C., then stirred at room temperature overnight, then the solvent was removed in vacuo and the residue was diluted with CH₂Cl₂ (30 mL) and washed with saturated solutions of Na₂CO₃ (20 mL) and brine (20 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 8e (25.50 mg, 61.8%) as pale yellow solid. ¹H NMR (500 MHz, CDCl₃) δ 8.94 (d, J=2.5 Hz, 1H), 8.40 (d, J=1.9 Hz, 1H), 7.16-7.11 (m, 1H), 7.08-7.02 (m, 1H), 7.00-6.92 (m, 1H), 3.99 (d, J=1.4 Hz, 3H), 3.51-3.45 (m, 1H), 3.28-3.16 (m, 1H), 2.72 (s, 3H), 2.56-2.49 (m, 1H), 2.34 (s, 3H), 2.08-1.82 (m, 4H). MS (ESI) m/z: 413.1 (M+1)⁺.

### Example 71. Synthesis of (R)-3-(3-fluoro-4-methoxyphenyl)-8-methyl-2-(1-methylpyrrolidin-2-yl)-6-nitroquinazolin-4 (3H)-one (8f)

Prepared by reductive amination of 7c.

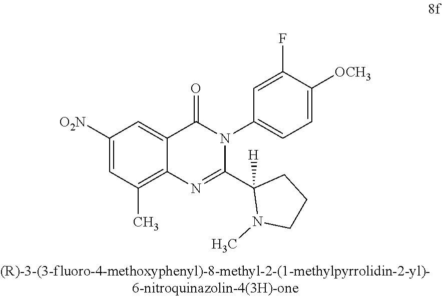 [SMILES: COc1ccc(-n2c([C@H]3CCCN3C)nc3c(C)cc([N+](=O)[O-])cc3c2=O)cc1F]

### Synthesis of (R)-3-(3-fluoro-4-methoxyphenyl)-8-methyl-2-(1-methylpyrrolidin-2-yl)-6-nitroquinazol-in-4 (3H)-one (8f)

Acetic acid (0.02 mL) was added to the solution of 7c (39.85 mg, 0.10 mmol) and formaldehyde solution 37.0% in H₂O (16.25 mg, 0.20 mmol) in MeOH (1.5 mL) and CH₂Cl₂ (1.5 mL), stirred at room temperature for 1 h, then sodium cyanoborohydride (9.45 mg, 0.15 mmol) was added to the reaction mixture at 0° C., then stirred at room temperature overnight, then the solvent was removed in vacuo and the residue was diluted with CH₂Cl₂ (30 mL) and washed with saturated solutions of Na₂CO₃ (20 mL) and brine (20 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 8f (23.80 mg, 57.8%) as pale yellow solid. ¹H NMR (500 MHz, CDCl₃) δ 8.93 (d, J=2.5 Hz, 1H), 8.40 (d, J=3.2 Hz, 1H), 7.18-7.11 (m, 1H), 7.08-7.02 (m, 1H), 7.01-6.95 (m, 1H), 3.99 (d, J=1.4 Hz, 3H), 3.51-3.41 (m, 1H), 3.35-3.24 (m, 1H), 2.72 (s, 3H), 2.61-2.51 (m, 1H), 2.34 (s, 3H), 2.09-2.00 (m, 2H), 1.90-1.75 (m, 2H). MS (ESI) m/z: 413.1 (M+1)⁺.

### Example 72. Synthesis of (R)-3-(4-chlorophenyl)-6-nitro-4-methyl-2-(pyrrolidin-2-yl) quinazolin-4 (3H)-one (7l)

Prepared as shown in Scheme 3

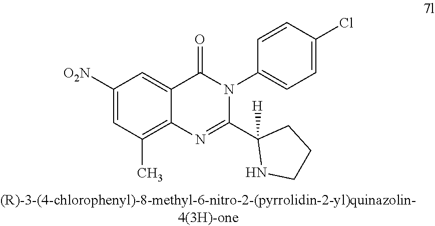 [SMILES: Cc1cc([N+](=O)[O-])cc2c(=O)n(-c3ccc(Cl)cc3)c([C@H]3CCCN3)nc12]

### Synthesis of (R)-3-(4-chlorophenyl)-6-nitro-4-methyl-2-(pyrrolidin-2-yl) quinazolin-4 (3H)-one (7l)

To a microwave vial containing 5-nitro-3-methylanthranilic acid (0.196 g, 1.0 mmol) and N-Boc-D-proline (0.255 g, 1.3 mmol) was added dry pyridine (3 mL). Triphenyl phosphite (0.8 mL, 3 mmol) was added to the vial, and the reaction mixture was heated at 100° C. with MWI for 40 min. After cooling the vial, 4-chloroaniline (0.153 g, 1.2 mmol) was added, and the reaction mixture was heated at 60° C. with MWI for 14 min. Pyridine was removed in vacuo, and the residue was diluted with CH₂Cl₂ (50 mL), and then the organic layer was washed sequentially with 10% citric acid (20 mL) and brine (50 mL). The separated organic extract was dried over MgSO₄, filtered, and concentrated in vacuo under reduced pressure. The crude mixture was purified by flash chromatography (3% Ethyl acetate/DCM) to afford tert-butyl (R)-2-(6-nitro-4-methyl-4-oxo-3-(4-chlorophenyl)-3,4-dihydroquinazolin-2-yl)pyrrolidine-1-carboxylate as a yellow solid. The solid (48.9 mg, 0.1 mmol) was dissolved in dry dioxane (1.0 mL), cooled to 0° C., and 4 M HCl/Dioxane (0.12 mL, 0.5 mmol) was added. After the addition was complete, the reaction mixture was stirred at rt for 8 h. Solvent was removed in vacuo under reduced pressure, and CH₂Cl₂ (10 mL) was added, followed by sequential washing with saturated Na₂CO₃ (5 mL) and brine (5 mL). The separated organic extract was dried over anhydrous MgSO₄, filtered and concentrated in vacuo under reduced pressure. The crude mixture was purified by flash chromatography (0% MeOH/CH₂Cl₂˜5% MeOH/CH₂Cl₂) to afford 71 (11 mg, 28%) as a yellow solid. ¹H NMR (500 MHz, chloroform-d) δ 8.93 (d, J=2.6 Hz, 1H), 8.41 (d, J=2.6 Hz, 1H), 7.64-7.47 (m, 2H), 7.29-7.25 (m, 2H), 7.22 (dd, J=8.9, 2.5 Hz, 1H), 3.88 (t, J=7.0 Hz, 1H), 3.28 (ddd, J=10.6, 7.3, 4.6 Hz, 1H), 2.90-2.79 (m, 1H), 2.70 (s, 3H), 1.92-1.73 (m, 4H). MS (ESI) m/z: 385.1 (M+1)⁺.

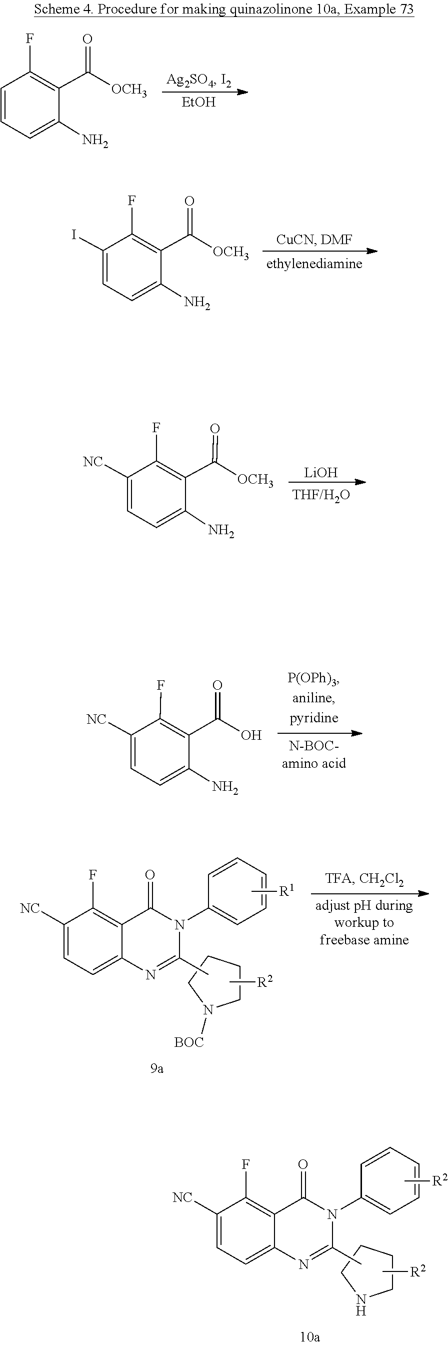

### Example 73. Synthesis of (R)-5-fluoro-3-(4-methoxyphenyl)-4-oxo-2-(pyrrolidin-2-yl)-3,4-dihydroquinazoline-6-carbonitrile (10a)

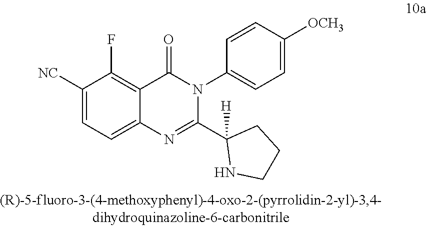 [SMILES: COc1ccc(-n2c([C@H]3CCCN3)nc3ccc(C#N)c(F)c3c2=O)cc1]

Step 1. Synthesis of methyl 6-amino-2-fluoro-3-iodobenzoate. A mixture of methyl-2-amino-6-fluorobenzoate (286.00 mg, 1.69 mmol), silver sulfate (526.94 mg, 1.69 mmol) and iodine (428.94 mg, 1.69 mmol) in ethanol (15 mL) was stirred at room temperature for 4 h. The reaction was filtered and concentrated under reduced pressure to afford residue, then washed with NaHCO₃ solution (20 mL), water (20 mL) and brine (20 mL), dried over anhydrous Na₂SO₄, and filtered, and the solvent was evaporated under reduced pressure. The residue was purified by flash chromatography (0% Ethyl acetate/Hexane 10% Ethyl acetate/Hexane) to afford methyl 6-amino-2-fluoro-3-iodobenzoate (0.27 g, 56.1%) as off-white solid: ¹H NMR (500 MHz, CDCl₃) δ 7.47 (dd, J=8.8, 6.5 Hz, 1H), 6.29 (dd, J=8.8, 1.0 Hz, 1H), 5.75 (s, 2H), 3.91 (s, 3H). MS (ESI) m/z: 296.1 (M+1)⁺.

Step 2. Synthesis of methyl 6-amino-3-cyano-2-fluorobenzoate. A mixture of methyl 6-amino-2-fluoro-3-iodobenzoate (0.68 g, 2.30 mmol), copper cyanide (0.26 g, 3.00 mmol) in DMF (10 mL) was heated at 150° C. for 10 h. After cooling to room temperature, and most of DMF was removed by rotavapor, water (40 mL) was added to reaction mixture, then followed by ethylenediamine (4 mL), and the mixture was stirred for 1 hour. The solids were removed by filtration, then extracted with ethyl acetate (2×200 mL) and water (50 mL). The combined organic layer was dried over with anhydrous Na₂SO₄, and filtered, then the solvent was evaporated under reduced pressure. The residue was purified by flash chromatography (0% Ethyl acetate/Hexane˜30% Ethyl acetate/Hexane) to afford methyl 6-amino-3-cyano-2-fluorobenzoate (0.26 g, 58.3%) as pale yellow solid: ¹H NMR (400 MHz, CDCl₃) δ 7.35 (dd, J=8.8, 6.6 Hz, 1H), 6.47 (dd, J=8.8, 1.0 Hz, 1H), 3.93 (s, 3H). MS (ESI) m/z: 195.1 (M+1)⁺.

Step 3. Synthesis of 6-amino-3-cyano-2-fluorobenzoic acid. At 0° C., lithium hydroxide (60.00 mg, 2.48 mmol) was added into the solution of methyl 6-amino-3-cyano-2-fluorobenzoate (0.24 g, 1.24 mmol) in THE (6 mL) and H₂O (6 mL), then stirred at room temperature overnight. The mixture was then adjusted to pH 2 with 1.0N HCl and extracted with ethyl acetate (3×60 mL). The combined organic layers were washed with brine (30 mL), separated, and dried over anhydrous Na₂SO₄, Concentration under reduced pressure afforded 6-amino-3-cyano-2-fluorobenzoic acid (0.22 g, 97.6%) as pale yellow solid. ¹H NMR (500 MHz, Acetone-d₆) δ 7.49 (dd, J=8.8, 7.0 Hz, 1H), 7.39 (s, 1H), 6.78 (d, J=8.9 Hz, 1H). MS (ESI) m/z: 181.1 (M+1)⁺.

Step 4. Synthesis of tert-butyl (R)-2-(6-cyano-5-fluoro-3-(4-methoxyphenyl)-4-oxo-3,4-dihydroquinazolin-2-yl)pyrrolidine-1-carboxylate (9a). 6-amino-3-cyano-2-fluorobenzoic acid (45.00 mg, 0.25 mmol) and Boc-D-proline (71.03 mg, 0.33 mmol) were dissolved in dry pyridine (1.5 mL). Triphenyl phosphite (0.20 mL, 0.75 mmol) was added to the mixture and the reaction mixture was heated at 100° C. with MWI for 40 min. 4-methoxyaniline (36.95 mg, 0.30 mmol) was added to the mixture and the reaction mixture was heated at 100° C. with MWI for 15 min. Pyridine was removed in vacuo and the residue was diluted with CH₂Cl₂ (40 mL) and washed with 1N HCl aqueous (20 mL). The organic layer was washed with H₂O (20 mL) and brine (20 mL), dried over anhydrous Na₂SO₄, and filtered, then the solvent was evaporated under reduced pressure. The residue was purified by flash chromatography (0% Ethyl acetate/Hexane˜40% Ethyl acetate/Hexane) to afford 9a (35.00 mg, 30.3%) as pale yellow solid. ¹H NMR (500 MHz, CDCl₃) δ 7.59 (dd, J=23.1, 8.5 Hz, 1H), 7.51-7.20 (m, 1H), 7.16-7.01 (m, 3H), 6.83 (dd, J=21.8, 8.5 Hz, 1H), 4.48 (ddd, J=36.8, 7.8, 4.1 Hz, 1H), 3.88 (d, J=10.9 Hz, 3H), 3.75-3.63 (m, 1H), 3.57-3.41 (m, 1H), 2.13-1.78 (m, 4H), 1.42 (s, 4H), 1.31 (s, 5H). MS (ESI) m/z: 465.1 (M+1)⁺.

Step 5. Synthesis of (R)-5-fluoro-3-(4-methoxyphenyl)-4-oxo-2-(pyrrolidin-2-yl)-3,4-dihydroquinazoline-6-carbonitrile (10a)

Under nitrogen, trifluoroacetic acid (0.12 mL, 1.60 mmol) was added to a solution of 9a (35.00 mg, 0.08 mmol) in dry CH₂Cl₂ (3 mL) at 0° C., then stirred at room temperature overnight, then the solvent was removed in vacuo and the residue was diluted with CH₂Cl₂ (20 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 10a (13.60 mg, 46.6%) as pale yellow solid. ¹H NMR (500 MHz, CDCl₃) δ 7.61 (d, J=8.4 Hz, 1H), 7.21 (d, J=7.9 Hz, 1H), 7.15 (d, J=7.9 Hz, 1H), 7.09-7.03 (m, 2H), 6.86 (d, J=8.4 Hz, 1H), 3.94-3.79 (m, 4H), 3.25 (d, J=4.4 Hz, 1H), 2.97 (s, 2H), 2.84-2.74 (m, 1H), 1.81-1.68 (m, 4H). MS (ESI) m/z: 365.1 (M+1)⁺.

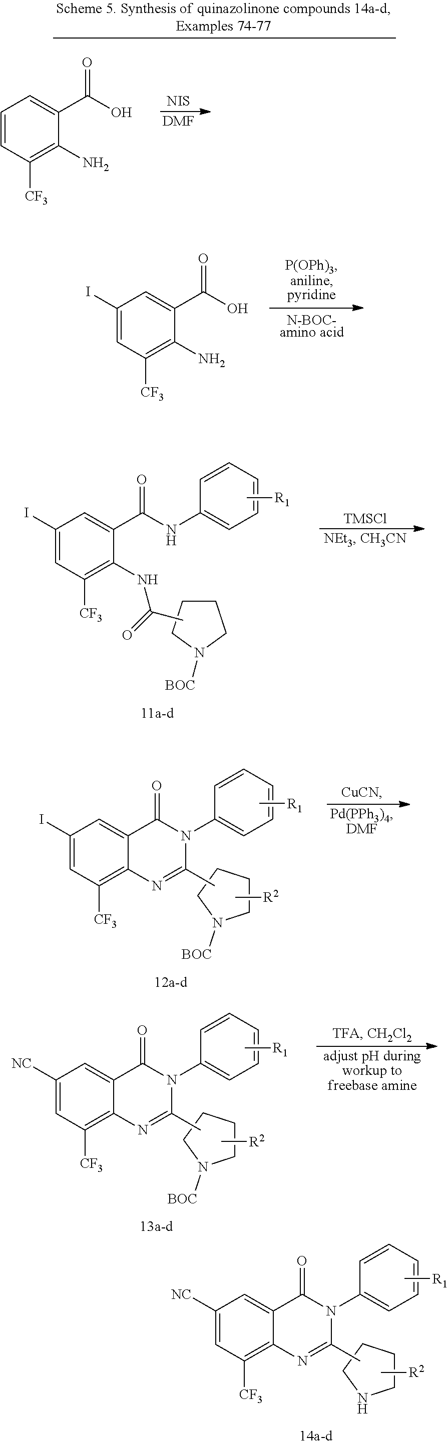

### Example 74. Synthesis of (R)-3-(4-methoxyphenyl)-4-oxo-2-(pyrrolidin-2-yl)-8-(trifluoromethyl)-3,4-dihydroquinazoline-6-carbonitrile (14a)

Prepared according to Scheme 5.

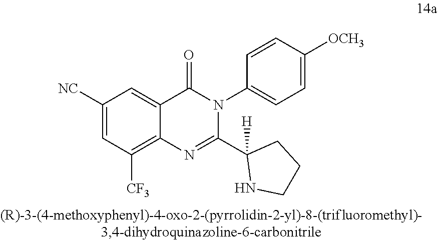 [SMILES: COc1ccc(-n2c([C@H]3CCCN3)nc3c(C(F)(F)F)cc(C#N)cc3c2=O)cc1]

Step 1. Synthesis of 2-amino-5-iodo-3-(trifluoromethyl)benzoic acid

A mixture of 2-amino-3-trifluoro-methylbenzoic acid (4.10 g, 20.00 mmol) and N-iodosuccinimide (4.75 g, 21.00 mmol) in DMF (50 mL) was heated at 65° C. for 6 h. After cooling to room temperature, the mixture was poured into ice water, and the resulting precipitate was collected by filtration. After washing the residue with water, the resulting solid was dissolved with acetone, then dried over with anhydrous Na₂SO₄, filtered, and then the solvent was evaporated under reduced pressure to afford 2-amino-5-iodo-3-(trifluoromethyl)benzoic acid (5.86 g, 88.6%) as brown solid. ¹H NMR (500 MHz, acetone-d₆) δ 8.39 (d, J=1.8 Hz, 1H), 7.89 (d, J=1.5 Hz, 1H), 7.06 (s, 2H). MS (ESI) m/z: 332.1 (M+1)⁺.

Step 2. Synthesis of tert-butyl (R)-2-((4-iodo-2-((4-methoxyphenyl) carbamoyl)-6-(trifluoromethyl)phenyl) carbamoyl) pyrrolidine-1-carboxylate (11a). 2-amino-5-iodo-3-(trifluoromethyl)benzoic acid (331.03 mg, 1.00 mmol) and Boc-D-proline (279.82 mg, 1.30 mmol) were dissolved in dry pyridine (5 mL). Triphenyl phosphite (0.79 mL, 3.00 mmol) was added to the mixture and the reaction mixture was heated at 100° C. with MWI for 40 min. 4-methoxyaniline (147.80 mg, 1.20 mmol) was added to the mixture and the reaction mixture was heated at 100° C. with MWI for 20 min. Pyridine was removed in vacuo and the residue was diluted with CH₂Cl₂ (180 mL) and washed with 1N HCl aqueous (40 mL). The organic layer was washed with H₂O (60 mL) and brine (60 mL), dried over anhydrous Na₂SO₄, and filtered, then the solvent was evaporated under reduced pressure. The residue was purified by flash chromatography (0% Ethyl acetate/Hexane˜40% Ethyl acetate/Hexane) to afford 11a (0.22 g, 34.5%) as off-white solid. ¹H NMR (400 MHz, CDCl₃) δ 9.18 (brs, 1H), 8.42 (s, 1H), 8.04 (brs, 1H), 7.90 (s, 1H), 7.38 (d, J=8.8 Hz, 2H), 6.78 (d, J=8.9 Hz, 2H), 4.41-4.25 (m, 1H), 3.73 (s, 3H), 3.36-3.16 (m, 2H), 2.29 (brs, 1H), 1.86-1.65 (m, 3H), 1.38 (s, 9H). MS (ESI) m/z: 534.1 (M+1−Boc)⁺.

Step 3. Synthesis of tert-butyl (R)-2-(6-iodo-3-(4-methoxyphenyl)-4-oxo-8-(trifluoromethyl)-3,4-dihydroquinazolin-2-yl) pyrrolidine-1-carboxylate (12a). 11a (200.00 mg, 0.32 mmol), triethylamine (0.66 mL, 4.75 mmol) and trimethylchlorosilane (0.40 mL, 3.20 mmol) were dissolved in dry acetonitrile (4 mL). The reaction mixture was heated at 150° C. with MWI for 15 min. After cooling to room temperature, the mixture was transferred to a larger flask and slowly quenched with saturated aqueous NaHCO₃ (60 mL). The resulting mixture was extracted with ethyl acetate (3×60 mL). The combined organic layers were washed with water (50 mL) and brine (50 mL), dried over anhydrous Na₂SO₄, and filtered, then the solvent was evaporated under reduced pressure. The residue was purified by flash chromatography (0% Ethyl acetate/Hexane 20% Ethyl acetate/Hexane) to afford 12a (0.11 g, 55.8%) as pale yellow oil. ¹H NMR (400 MHz, CDCl₃) δ 8.68 (dd, J=10.1, 1.7 Hz, 1H), 8.21 (dd, J=16.1, 1.4 Hz, 1H), 7.51-7.15 (m, 1H), 7.06-6.99 (m, 2H), 6.98-6.91 (m, 1H), 4.51-4.36 (m, 1H), 3.80 (d, J=9.1 Hz, 3H), 3.75-3.64 (m, 1H), 3.43-3.29 (m, 1H), 2.11-1.69 (m, 4H), 1.32 (s, 6H), 1.20 (s, 3H). MS (ESI) m/z: 616.1 (M+1)⁺.

Step 4. Synthesis of tert-butyl (R)-2-(6-cyano-3-(4-methoxyphenyl)-4-oxo-8-(trifluoromethyl)-3,4-dihydroquinazolin-2-yl) pyrrolidine-1-carboxylate (13a). A mixture of 12a (82.40 mg, 0.13 mmol), copper cyanide (12.81 mg, 0.14 mmol) and tetrakis(triphenylphosphine) palladium (0) (8.09 mg, 0.0070 mmol) in DMF (1 mL) was degassed and flushed with nitrogen. The mixture was heated at 100° C. for 4 h under nitrogen atmosphere. After cooling to room temperature, the mixture was diluted with H₂O (10 mL) and extracted with ethyl acetate (2×20 mL). The combined organic layers were washed with H₂O (20 mL) and brine (20 mL), dried over anhydrous Na₂SO₄, and filtered, then the solvent was evaporated under reduced pressure. The residue was purified by flash chromatography (0% Ethyl acetate/Hexane˜40% Ethyl acetate/Hexane) to afford 13a (34.16 mg, 53.8%) as pale yellow oil. ¹H NMR (400 MHz, CDCl₃) δ 8.67 (dd, J=10.1, 1.6 Hz, 1H), 8.16 (d, J=18.0 Hz, 1H), 7.44 (dd, J=8.7, 2.5 Hz, 1H), 7.07-6.94 (m, 3H), 4.55-4.41 (m, 1H), 3.82 (d, J=9.3 Hz, 3H), 3.76-3.65 (m, 1H), 3.48-3.33 (m, 1H), 2.08-1.74 (m, 4H), 1.33 (s, 6H), 1.21 (s, 3H). MS (ESI) m/z: 515.1 (M+1)⁺.

Step 5. Synthesis of (R)-3-(4-methoxyphenyl)-4-oxo-2-(pyrrolidin-2-yl)-8-(trifluoromethyl)-3,4-dihydroquinazoline-6-carbonitrile (14a)

Under nitrogen, trifluoroacetic acid (0.09 mL, 1.20 mmol) was added to a solution of 13c (30.00 mg, 0.06 mmol) in dry CH₂Cl₂ (1 mL) at 0° C., then stirred at room temperature overnight, then the solvent was removed in vacuo and the residue was diluted with CH₂Cl₂ (10 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 14a (16.10 mg, 64.8%) as off-white solid. ¹H NMR (500 MHz, CDCl₃) δ 8.67 (d, J=1.4 Hz, 1H), 8.18 (s, 1H), 7.14 (d, J=7.9 Hz, 1H), 7.08 (d, J=7.6 Hz, 1H), 7.00 (d, J=9.0 Hz, 2H), 3.89 (t, J=6.7 Hz, 1H), 3.82 (s, 3H), 3.23-3.13 (m, 1H), 2.80-2.73 (m, 1H), 2.22 (brs, 1H), 1.81-1.65 (m, 4H). MS (ESI) m/z: 415.1 (M+1)⁺.

### Example 75. Synthesis of (S)-3-(4-methoxyphenyl)-4-oxo-2-(pyrrolidin-2-yl)-8-(trifluoromethyl)-3,4-dihydroquinazoline-6-carbonitrile (14b)

Prepared according to Scheme 5.

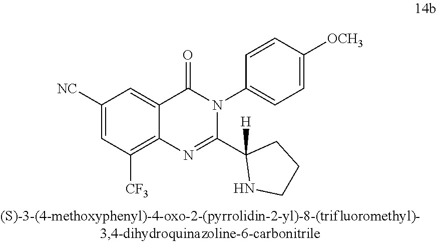 [SMILES: COc1ccc(-n2c([C@@H]3CCCN3)nc3c(C(F)(F)F)cc(C#N)cc3c2=O)cc1]

Step 1. Synthesis of tert-butyl (S)-2-((4-iodo-2-((4-methoxyphenyl) carbamoyl)-6-(trifluoromethyl)phenyl) carbamoyl) pyrrolidine-1-carboxylate (11b). 2-amino-5-iodo-3-(trifluoromethyl)benzoic acid (496.55 mg, 1.50 mmol) and Boc-L-proline (419.75 mg, 1.95 mmol) were dissolved in dry pyridine (6 mL). Triphenyl phosphite (1.20 mL, 4.50 mmol) was added to the mixture and the reaction mixture was heated at 100° C. with MWI for 40 min. 4-methoxyaniline (221.68 mg, 1.80 mmol) was added to the mixture and the reaction mixture was heated at 100° C. with MWI for 20 min. Pyridine was removed in vacuo and the residue was diluted with CH₂Cl₂ (160 mL) and washed with 1N HCl aqueous (60 mL). The organic layer was washed with H₂O (80 mL) and brine (80 mL), dried over anhydrous Na₂SO₄, and filtered, then the solvent was evaporated under reduced pressure. The residue was purified by flash chromatography (0% Ethyl acetate/Hexane˜40% Ethyl acetate/Hexane) to afford 11b (0.30 g, 31.6%) as off-white solid. ¹H NMR (400 MHz, CDCl₃) δ 9.16 (brs, 1H), 8.41 (s, 1H), 8.05 (brs, 1H), 7.86 (s, 1H), 7.36 (d, J=8.8 Hz, 2H), 6.78 (d, J=8.9 Hz, 2H), 4.45-4.28 (m, 1H), 3.75 (s, 3H), 3.41-3.18 (m, 2H), 2.31 (brs, 1H), 1.86-1.61 (m, 3H), 1.39 (s, 9H). MS (ESI) m/z: 534.1 (M+1−Boc)⁺.

Step 2. Synthesis of tert-butyl (S)-2-(6-iodo-3-(4-methoxyphenyl)-4-oxo-8-(trifluoromethyl)-3,4-dihydroquin-azolin-2-yl) pyrrolidine-1-carboxylate (12b). 11b (300.00 mg, 0.46 mmol), triethylamine (0.96 mL, 6.90 mmol) and trimethylchlorosilane (0.58 mL, 4.60 mmol) were dissolved in dry acetonitrile (5 mL). The reaction mixture was heated at 150° C. with MWI for 15 min. After cooling to room temperature, the mixture was transferred to a larger flask and slowly quenched with saturated aqueous NaHCO₃ (50 mL). The resulting mixture was extracted with ethyl acetate (3×60 mL). The combined organic layers were washed with water (50 mL) and brine (50 mL), dried over anhydrous Na₂SO₄, and filtered, then the solvent was evaporated under reduced pressure. The residue was purified by flash chromatography (0% Ethyl acetate/Hexane 20% Ethyl acetate/Hexane) to afford 12b (0.12 g, 43.5%) as off-white solid. ¹H NMR (400 MHz, CDCl₃) δ 8.69 (dd, J=9.8, 1.9 Hz, 1H), 8.21 (dd, J=16.2, 1.6 Hz, 1H), 7.45-7.11 (m, 1H), 7.06-6.94 (m, 3H), 4.51-4.38 (m, 1H), 3.81 (d, J=9.2 Hz, 3H), 3.75-3.66 (m, 1H), 3.45-3.29 (m, 1H), 2.08-1.71 (m, 4H), 1.33 (s, 6H), 1.21 (s, 3H). MS (ESI) m/z: 616.1 (M+1)⁺.

Step 3. Synthesis of tert-butyl (S)-2-(6-cyano-3-(4-methoxyphenyl)-4-oxo-8-(trifluoromethyl)-3,4-dihydroquinazolin-2-yl) pyrrolidine-1-carboxylate (13b). A mixture of 12b (98.60 mg, 0.16 mmol), Copper cyanide (16.12 mg, 0.18 mmol) and tetrakis(triphenylphosphine) palladium (0) (9.24 mg, 0.0080 mmol) in DMF (1.5 mL) was degassed and flushed with nitrogen. The mixture was heated at 100° C. for 4 h under nitrogen atmosphere. After cooling to room temperature, the mixture was diluted with H₂O (10 mL) and extracted with ethyl acetate (2×20 mL). The combined organic layers were washed with H₂O (20 mL) and brine (20 mL), dried over anhydrous Na₂SO₄, and filtered, then the solvent was evaporated under reduced pressure. The residue was purified by flash chromatography (0% Ethyl acetate/Hexane˜40% Ethyl acetate/Hexane) to afford 13b (52.80 mg, 64.1%) as colorless oil. ¹H NMR (400 MHz, CDCl₃) δ 8.65 (dd, J=10.1, 1.8 Hz, 1H), 8.15 (d, J=18.0 Hz, 1H), 7.45 (dd, J=8.7, 2.5 Hz, 1H), 7.11-6.95 (m, 3H), 4.55-4.42 (m, 1H), 3.81 (d, J=9.3 Hz, 3H), 3.81-3.65 (m, 1H), 3.48-3.35 (m, 1H), 2.12-1.75 (m, 4H), 1.34 (s, 6H), 1.23 (s, 3H). MS (ESI) m/z: 515.1 (M+1)⁺.

Step 4. Synthesis of (S)-3-(4-methoxyphenyl)-4-oxo-2-(pyrrolidin-2-yl)-8-(trifluoromethyl)-3,4-dihydroquinazoline-6-carbonitrile (14b)

Under nitrogen, trifluoroacetic acid (0.14 mL, 1.80 mmol) was added to a solution of 13b (45.00 mg, 0.09 mmol) in dry CH₂Cl₂ (1.5 mL) at 0° C., then stirred at room temperature overnight, then the solvent was removed in vacuo and the residue was diluted with CH₂Cl₂ (10 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 14b (26.06 mg, 69.8%) as off-white solid. ¹H NMR (400 MHz, CDCl₃) δ 8.66 (d, J=1.8 Hz, 1H), 8.17 (d, J=1.4 Hz, 1H), 7.14 (d, J=7.8 Hz, 1H), 7.08 (dd, J=9.6, 1.9 Hz, 1H), 7.00 (d, J=9.1 Hz, 2H), 3.91-3.86 (m, 1H), 3.82 (s, 3H), 3.25-3.12 (m, 1H), 2.86-2.68 (m, 1H), 2.33 (brs, 1H), 1.81-1.68 (m, 4H). MS (ESI) m/z: 415.1 (M+1)⁺.

### Example 76. Synthesis of (R)-3-(3-fluoro-4-methoxyphenyl)-4-oxo-2-(pyrrolidin-2-yl)-8-(trifluoromethyl)-3,4-dihydroquinazoline-6-carbonitrile (14c)

Prepared according to Scheme 5.

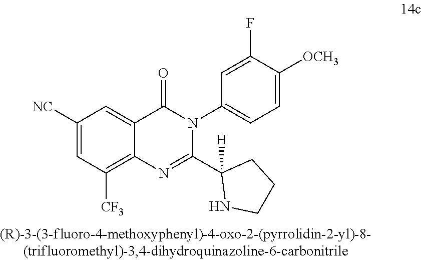 [SMILES: COc1ccc(-n2c([C@H]3CCCN3)nc3c(C(F)(F)F)cc(C#N)cc3c2=O)cc1F]

Step 1. Synthesis of tert-butyl (R)-2-((4-iodo-2-((3-fluoro-4-methoxyphenyl) carbamoyl)-6-(trifluoromethyl)phenyl) carbamoyl) pyrrolidine-1-carboxylate (tic). 2-amino-5-iodo-3-(trifluoromethyl)benzoic acid (496.55 mg, 1.50 mmol) and Boc-D-proline (419.75 mg, 1.95 mmol) were dissolved in dry pyridine (6 mL). Triphenyl phosphite (1.18 mL, 4.50 mmol) was added to the mixture and the reaction mixture was heated at 100° C. with MWI for 40 min. 3-Fluoro-4-methoxyaniline (254.05 mg, 1.80 mmol) was added to the mixture and the reaction mixture was heated at 100° C. with MWI for 20 min. Pyridine was removed in vacuo and the residue was diluted with CH₂Cl₂ (200 mL) and washed with 1N HCl aqueous (60 mL). The organic layer was washed with H₂O (80 mL) and brine (80 mL), dried over anhydrous Na₂SO₄, and filtered, then the solvent was evaporated under reduced pressure. The residue was purified by flash chromatography (0% Ethyl acetate/Hexane to 40% Ethyl acetate/Hexane) to afford 11c (0.19 g, 19.5%) as pale yellow solid. ¹H NMR (400 MHz, CDCl₃) δ 9.25 (brs, 1H), 8.60 (s, 1H), 8.10 (s, 1H), 7.99 (s, 1H), 7.49 (dd, J=12.7, 1.9 Hz, 1H), 7.15 (d, J=8.7 Hz, 1H), 6.89 (t, J=8.9 Hz, 1H), 4.46-4.38 (m, 1H), 3.88 (s, 3H), 3.36-3.24 (m, 2H), 2.38 (brs, 1H), 1.88-1.68 (m, 3H), 1.46 (s, 9H). MS (ESI) m/z: 552.1 (M+1−Boc)⁺.

Step 2. Synthesis of tert-butyl (R)-2-(6-iodo-3-(3-fluoro-4-methoxyphenyl)-4-oxo-8-(trifluoromethyl)-3,4-dihy-droquinazolin-2-yl) pyrrolidine-1-carboxylate (12c). 11c (0.28 g, 0.43 mmol), triethylamine (0.90 mL, 6.45 mmol) and trimethylchlorosilane (0.55 mL, 4.30 mmol) were dissolved in dry acetonitrile (5 mL). The reaction mixture was heated at 150° C. with MWI for 15 min. After cooling to room temperature, the mixture was transferred to a larger flask and slowly quenched with saturated aqueous NaHCO₃ (80 mL). The resulting mixture was extracted with ethyl acetate (3×60 mL). The combined organic layers were washed with water (60 mL) and brine (60 mL), dried over anhydrous Na₂SO₄, and filtered, then the solvent was evaporated under reduced pressure. The residue was purified by flash chromatography (0% Ethyl acetate/Hexane˜20% Ethyl acetate/Hexane) to afford 12c (80.10 mg, 29.4%) as pale yellow oil. ¹H NMR (400 MHz, CDCl₃) δ 8.75 (d, J=10.5 Hz, 1H), 8.30 (d, J=16.9 Hz, 1H), 7.45-7.11 (m, 2H), 6.95 (dd, J=13.7, 11.0 Hz, 1H), 4.60-4.44 (m, 1H), 4.01-3.94 (m, 3H), 3.85-3.72 (m, 1H), 3.51-3.36 (m, 1H), 2.11-1.72 (m, 4H), 1.40 (s, 6H), 1.28 (s, 3H). MS (ESI) m/z:634.1 (M+1)⁺.

Step 3. Synthesis of tert-butyl (R)-2-(6-cyano-3-(3-fluoro-4-methoxyphenyl)-4-oxo-8-(trifluoromethyl)-3,4-dih-ydroqu-inazolin-2-yl) pyrrolidine-1-carboxylate (13c). A mixture of 12c (80.00 mg, 0.13 mmol), Copper cyanide (12.54 mg, 0.14 mmol) and tetrakis(triphenylphosphine) palladium (0) (6.93 mg, 0.0060 mmol) in DMF (1 mL) was degassed and flushed with nitrogen. The mixture was heated at 100° C. for 4 h under nitrogen atmosphere. After cooling to room temperature, the mixture was diluted with H₂O (10 mL) and extracted with ethyl acetate (2×20 mL). The combined organic layers were washed with H₂O (20 mL) and brine (20 mL), dried over anhydrous Na₂SO₄, and filtered, then the solvent was evaporated under reduced pressure. The residue was purified by flash chromatography (0% Ethyl acetate/Hexane˜40% Ethyl acetate/Hexane) to afford 13c (26.80 mg, 38.7%) as pale yellow oil. ¹H NMR (400 MHz, CDCl₃) δ 8.73 (d, J=10.8 Hz, 1H), 8.23 (d, J=18.6 Hz, 1H), 7.45-7.08 (m, 2H), 6.97 (dt, J=12.8, 6.4 Hz, 1H), 4.61-4.48 (m, 1H), 4.02-3.95 (m, 3H), 3.84-3.71 (m, 1H), 3.52-3.45 (m, 1H), 2.18-1.86 (m, 4H), 1.40 (d, J=3.8 Hz, 6H), 1.27 (d, J=11.8 Hz, 3H). MS (ESI) m/z: 533.1 (M+1)⁺.

Step 4. Synthesis of (R)-3-(3-fluoro-4-methoxyphenyl)-4-oxo-2-(pyrrolidin-2-yl)-8-(trifluoromethyl)-3,4-dihydroquinazoline-6-carbonitrile (14c)

Under nitrogen, trifluoroacetic acid (0.08 mL, 1.00 mmol) was added to a solution of 13c (26.00 mg, 0.05 mmol) in dry CH₂Cl₂ (1 mL) at 0° C., then stirred at room temperature overnight, then the solvent was removed in vacuo and the residue was diluted with CH₂Cl₂ (10 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 14c (13.80 mg, 63.8%) as off-white solid. ¹H NMR (500 MHz, CDCl₃) δ 8.74 (d, J=1.6 Hz, 1H), 8.26 (s, 1H), 7.16-7.11 (m, 1H), 7.11-7.06 (m, 1H), 7.03-6.98 (m, 1H), 3.99-3.94 (m, 4H), 3.31-3.18 (m, 1H), 2.91-2.78 (m, 1H), 2.39 (brs, 1H), 1.92-1.78 (m, 4H). MS (ESI) m/z: 433.1 (M+1)⁺.

### Example 77. Synthesis of (S)-3-(3-fluoro-4-methoxyphenyl)-4-oxo-2-(pyrrolidin-2-yl)-8-(trifluoromethyl)-3,4-dihydroquinazoline-6-carbonitrile (14d)

Prepared according to Scheme 5.

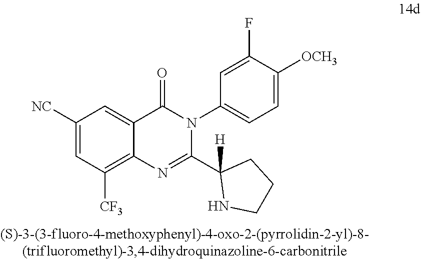 [SMILES: COc1ccc(-n2c([C@@H]3CCCN3)nc3c(C(F)(F)F)cc(C#N)cc3c2=O)cc1F]

Step 1. Synthesis of tert-butyl (S)-2-((4-iodo-2-((3-fluoro-4-methoxyphenyl) carbamoyl)-6-(trifluoromethyl)phenyl) carbamoyl) pyrrolidine-1-carboxylate (11d). 2-amino-5-iodo-3-(trifluoromethyl)benzoic acid (496.55 mg, 1.50 mmol) and Boc-L-proline (419.75 mg, 1.95 mmol) were dissolved in dry pyridine (6 mL). Triphenyl phosphite (1.18 mL, 4.50 mmol) was added to the mixture and the reaction mixture was heated at 100° C. with MWI for 40 min. 3-Fluoro-4-methoxyaniline (254.05 mg, 1.80 mmol) was added to the mixture and the reaction mixture was heated at 100° C. with MWI for 20 min. Pyridine was removed in vacuo and the residue was diluted with CH₂Cl₂ (200 mL) and washed with 1N HCl aqueous (60 mL). The organic layer was washed with H₂O (80 mL) and brine (80 mL), dried over anhydrous Na₂SO₄, and filtered, then the solvent was evaporated under reduced pressure. The residue was purified by flash chromatography (0% Ethyl acetate/Hexane˜40% Ethyl acetate/Hexane) to afford 11d (0.11 g, 11.3%) as pale yellow solid. ¹H NMR (400 MHz, CDCl₃) δ 9.24 (brs, 1H), 8.56 (s, 1H), 8.11 (s, 1H), 7.84 (s, 1H), 7.51 (dd, J=12.8, 1.8 Hz, 1H), 7.18 (d, J=8.8 Hz, 1H), 6.86 (t, J=8.8 Hz, 1H), 4.51-4.38 (m, 1H), 3.86 (s, 3H), 3.38-3.25 (m, 2H), 2.41 (brs, 1H), 1.91-1.65 (m, 3H), 1.45 (s, 9H). MS (ESI) m/z: 552.1 (M+1−Boc)⁺.

Step 2. Synthesis of tert-butyl (S)-2-(6-iodo-3-(3-fluoro-4-methoxyphenyl)-4-oxo-8-(trifluoromethyl)-3,4-dihy-droquinazolin-2-yl) pyrrolidine-1-carboxylate (12d). 11d (0.28 g, 0.43 mmol), triethylamine (0.90 mL, 6.45 mmol) and trimethylchlorosilane (0.55 mL, 4.30 mmol) were dissolved in dry acetonitrile (5 mL). The reaction mixture was heated at 150° C. with MWI for 15 min. After cooling to room temperature, the mixture was transferred to a larger flask and slowly quenched with saturated aqueous NaHCO₃ (80 mL). The resulting mixture was extracted with ethyl acetate (3×60 mL). The combined organic layers were washed with water (60 mL) and brine (60 mL), dried over anhydrous Na₂SO₄, and filtered, then the solvent was evaporated under reduced pressure. The residue was purified by flash chromatography (0% Ethyl acetate/Hexane˜20% Ethyl acetate/Hexane) to afford 12d (82.60 mg, 30.3%) as pale yellow oil. ¹H NMR (400 MHz, CDCl₃) δ 8.76 (d, J=10.4 Hz, 1H), 8.31 (d, J=16.6 Hz, 1H), 7.51-7.06 (m, 2H), 6.96 (dd, J=13.8, 11.0 Hz, 1H), 4.61-4.45 (m, 1H), 4.01-3.95 (m, 3H), 3.86-3.71 (m, 1H), 3.53-3.35 (m, 1H), 2.11-1.76 (m, 4H), 1.41 (s, 6H), 1.31 (s, 3H). MS (ESI) m/z: 634.1 (M+1)⁺.

Step 3. Synthesis of tert-butyl (S)-2-(6-cyano-3-(3-fluoro-4-methoxyphenyl)-4-oxo-8-(trifluoromethyl)-3,4-dihydroquinazolin-2-yl) pyrrolidine-1-carboxylate (13d). A mixture of 12d (80.00 mg, 0.13 mmol), copper cyanide (12.54 mg, 0.14 mmol) and tetrakis (triphenylphosphine) palladium (0) (6.93 mg, 0.0060 mmol) in DMF (1 mL) was degassed and flushed with nitrogen. The mixture was heated at 100° C. for 4 h under nitrogen atmosphere. After cooling to room temperature, the mixture was diluted with H₂O (10 mL) and extracted with ethyl acetate (2×20 mL). The combined organic layers were washed with H₂O (20 mL) and brine (20 mL), dried over anhydrous Na₂SO₄, and filtered, then the solvent was evaporated under reduced pressure. The residue was purified by flash chromatography (0% Ethyl acetate/Hexane˜40% Ethyl acetate/Hexane) to afford 13d (23.60 mg, 34.1%) as pale yellow oil. ¹H NMR (400 MHz, CDCl₃) δ 8.75 (d, J=10.6 Hz, 1H), 8.24 (d, J=18.6 Hz, 1H), 7.45-7.06 (m, 2H), 6.97 (dt, J=12.6, 6.5 Hz, 1H), 4.61-4.45 (m, 1H), 4.05-3.95 (m, 3H), 3.85-3.71 (m, 1H), 3.54-3.45 (m, 1H), 2.18-1.86 (m, 4H), 1.41 (d, J=3.8 Hz, 6H), 1.28 (d, J=11.8 Hz, 3H). MS (ESI) m/z: 533.1 (M+1)⁺.

Step 4. Synthesis of (S)-3-(3-fluoro-4-methoxyphenyl)-4-oxo-2-(pyrrolidin-2-yl)-8-(trifluoromethyl)-3,4-dihydroquinazoline-6-carbonitrile (14d)

Under nitrogen, trifluoroacetic acid (0.06 mL, 0.80 mmol) was added to a solution of 13d (22.00 mg, 0.04 mmol) in dry CH₂Cl₂ (1 mL) at 0° C., then stirred at room temperature overnight, then the solvent was removed in vacuo and the residue was diluted with CH₂Cl₂ (10 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 14d (10.60 mg, 61.3%) as off-white solid. ¹H NMR (500 MHz, CDCl₃) δ 8.75 (d, J=1.6 Hz, 1H), 8.26 (s, 1H), 7.18-7.08 (m, 1H), 7.12-7.05 (m, 1H), 7.06-6.98 (m, 1H), 3.98-3.91 (m, 4H), 3.31-3.16 (m, 1H), 2.91-2.76 (m, 1H), 2.38 (brs, 1H), 1.94-1.78 (m, 4H). MS (ESI) m/z: 433.1 (M+1)⁺.

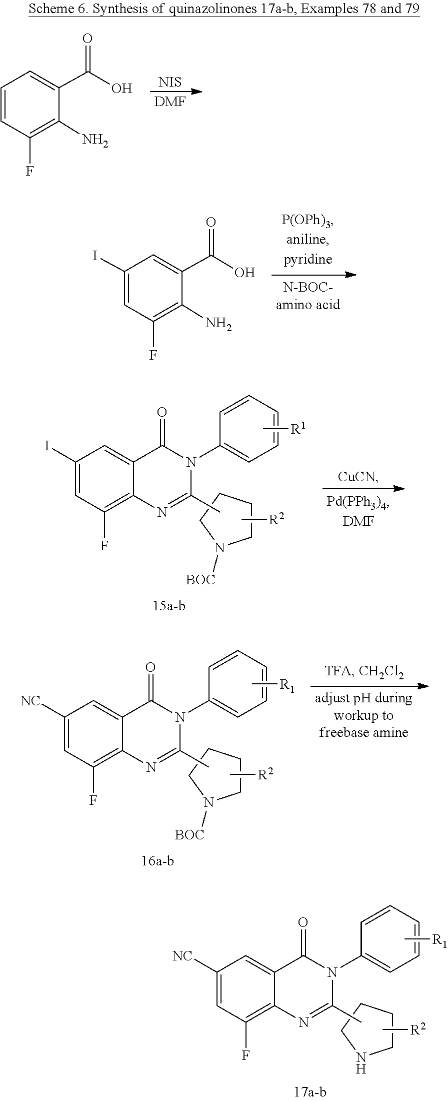

### Example 78. Synthesis of (R)-8-fluoro-3-(4-methoxyphenyl)-4-oxo-2-(pyrrolidin-2-yl)-3,4-dihydroquinazoline-6-carbonitrile (17a)

Prepared according to Scheme 6.

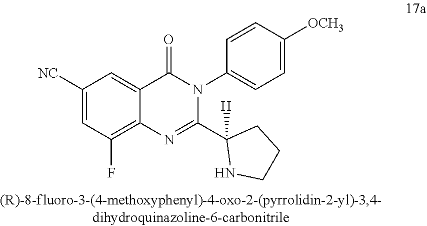 [SMILES: COc1ccc(-n2c([C@H]3CCCN3)nc3c(F)cc(C#N)cc3c2=O)cc1]

Step 1. Synthesis of 2-amino-3-fluoro-5-iodobenzoic acid

A mixture of 2-amino-3-fluorobenzoic acid (1.55 g, 10.00 mmol) and N-iodosuccinimide (2.68 g, 12.00 mmol) in DMF (25 mL) was stirred at room temperature for 4 h, then the mixture was poured into ice water and the resulting precipitate was collected by filtration. After washing the residue with water, the resulting solid was dissolved with acetone, then dried over with anhydrous Na₂SO₄, and filtered, then the solvent was evaporated under reduced pressure to afford 2-amino-3-fluoro-5-iodobenzoic acid (1.86 g, 66.1%) as pale brown solid. ¹H NMR (400 MHz, DMSO-d₆) δ 7.81 (s, 1H), 7.57 (dd, J=10.8, 1.8 Hz, 1H). MS (ESI) m/z: 282.1 (M+1)⁺.

Step 2. Synthesis of tert-butyl (R)-2-(8-fluoro-6-iodo-3-(4-methoxyphenyl)-4-oxo-3,4-dihydroquinazolin-2-yl)-pyrrolidine-1-carboxylate (15a). 2-amino-3-fluoro-5-iodobenzoic acid (281.02 mg, 1.00 mmol) and Boc-D-proline (280.00 mg, 1.30 mmol) were dissolved in dry pyridine (4 mL). Triphenyl phosphite (0.78 mL, 3.00 mmol) was added to the mixture and the reaction mixture was heated at 100° C. with MWI for 40 min. 4-methoxyaniline (148.00 mg, 1.20 mmol) was added to the mixture and the reaction mixture was heated at 100° C. with MWI for 30 min. Pyridine was removed in vacuo and the residue was diluted with CH₂Cl₂ (160 mL) and washed with 1N HCl aqueous (100 mL). The organic layer was washed with H₂O (100 mL) and brine (100 mL), dried over anhydrous Na₂SO₄, and filtered, then the solvent was evaporated under reduced pressure. The residue was purified by flash chromatography (0% Ethyl acetate/Hexane˜50% Ethyl acetate/Hexane) to afford 15a (211.60 mg, 37.5%) as pale pink solid. ¹H NMR (500 MHz, CDCl₃) δ 8.37 (d, J=13.0 Hz, 1H), 7.75 (ddd, J=23.0, 8.9, 1.8 Hz, 1H), 7.48-7.21 (m, 1H), 7.16-7.01 (m, 3H), 4.51 (ddd, J=45.4, 7.9, 3.9 Hz, 1H), 3.88 (d, J=10.8 Hz, 3H), 3.78-3.69 (m, 1H), 3.56-3.38 (m, 1H), 2.13-1.80 (m, 4H), 1.42 (s, 5H), 1.28 (s, 4H). MS (ESI) m/z: 566.1 (M+1)⁺.

Step 3. Synthesis of tert-butyl (R)-2-(6-cyano-8-fluoro-3-(4-methoxyphenyl)-4-oxo-3,4-dihydroquinazolin-2-yl) pyrrolidine-1-carboxylate (16a)

A mixture of 15a (100.00 mg, 0.18 mmol), Copper cyanide (17.95 mg, 0.20 mmol) and tetrakis(triphenylphosphine) palladium (0) (10.40 mg, 0.0090 mmol) in DMF (2 mL) was degassed and flushed with nitrogen. The mixture was heated at 100° C. for 4 h under nitrogen atmosphere. After cooling to room temperature, the mixture was diluted with H₂O (20 mL) and extracted with ethyl acetate (2×40 mL). The combined organic layers were washed with H₂O (40 mL) and brine (40 mL), dried over anhydrous Na₂SO₄, and filtered, then the solvent was evaporated under reduced pressure. The residue was purified by flash chromatography (0% Ethyl acetate/Hexane˜40% Ethyl acetate/Hexane) to afford 16a (51.86 mg, 62.1%) as colorless oil. ¹H NMR (400 MHz, CDCl₃) δ 8.35 (d, J=10.8 Hz, 1H), 7.64 (ddd, J=20.6, 9.0, 1.4 Hz, 1H), 7.48-7.18 (m, 1H), 7.14-7.01 (m, 3H), 4.61-4.56 (m, 1H), 3.89 (d, J=8.6 Hz, 3H), 3.76-3.68 (m, 1H), 3.61-3.41 (m, 1H), 2.16-1.81 (m, 4H), 1.42 (s, 6H), 1.29 (s, 3H). MS (ESI) m/z: 465.1 (M+1)⁺.

Step 4. Synthesis of (R)-8-fluoro-3-(4-methoxyphenyl)-4-oxo-2-(pyrrolidin-2-yl)-3,4-dihydroquinazoline-6-carbonitrile (17a)

Under nitrogen, trifluoroacetic acid (0.16 mL, 2.20 mmol) was added to a solution of 16a (50.00 mg, 0.11 mmol) in dry CH₂Cl₂ (2 mL) at 0° C., then stirred at room temperature overnight, then the solvent was removed in vacuo and the residue was diluted with CH₂Cl₂ (30 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂ to 5% MeOH/CH₂Cl₂) to afford 17a (16.80 mg, 42.0%) as pale yellow solid. ¹H NMR (500 MHz, CDCl₃) δ 8.36 (s, 1H), 7.67 (dd, J=9.1, 1.7 Hz, 1H), 7.22-7.19 (m, 1H), 7.18-7.13 (m, 1H), 7.11-7.05 (m, 2H), 3.89 (s, 3H), 3.28 (dd, J=10.9, 4.9 Hz, 1H), 3.11-3.06 (m, 1H), 2.85-2.76 (m, 1H), 1.86-1.75 (m, 4H). MS (ESI) m/z: 365.1 (M+1)⁺.

### Example 79. Synthesis of (S)-8-fluoro-3-(4-methoxyphenyl)-4-oxo-2-(pyrrolidin-2-yl)-3,4-dihydroquinazoline-6-carbonitrile (17b)

Prepared according to Scheme 6.

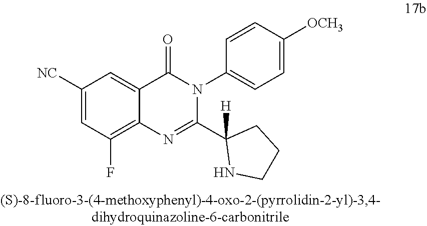 [SMILES: COc1ccc(-n2c([C@@H]3CCCN3)nc3c(F)cc(C#N)cc3c2=O)cc1]

Step 1. Synthesis of tert-butyl (S)-2-(8-fluoro-6-iodo-3-(4-methoxyphenyl)-4-oxo-3,4-dihydroquinazolin-2-yl)-pyrrolidine-1-carboxylate (15b). 2-amino-3-fluoro-5-iodobenzoic acid (281.02 mg, 1.00 mmol) and Boc-L-proline (280.00, 1.30 mmol) were dissolved in dry pyridine (4 mL). Triphenyl phosphite (0.78 mL, 3.00 mmol) was added to the mixture and the reaction mixture was heated at 100° C. with MWI for 40 min. 4-methoxyaniline (148.00 mg, 1.20 mmol) was added to the mixture and the reaction mixture was heated at 100° C. with MWI for 30 min. Pyridine was removed in vacuo and the residue was diluted with CH₂Cl₂ (160 mL) and washed with 1N HCl aqueous (100 mL). The organic layer was washed with H₂O (100 mL) and brine (100 mL), dried over anhydrous Na₂SO₄, and filtered, then the solvent was evaporated under reduced pressure. The residue was purified by flash chromatography (0% Ethyl acetate/Hexane˜50% Ethyl acetate/Hexane) to afford 15b (228.90 mg, 40.5%) as pale yellow solid. ¹H NMR (500 MHz, CDCl₃) δ 8.36 (d, J=12.8 Hz, 1H), 7.76 (ddd, J=23.0, 8.8, 1.8 Hz, 1H), 7.48-7.18 (m, 1H), 7.16-7.01 (m, 3H), 4.48 (ddd, J=45.4, 7.9, 3.9 Hz, 1H), 3.86 (d, J=10.8 Hz, 3H), 3.81-3.69 (m, 1H), 3.56-3.38 (m, 1H), 2.13-1.80 (m, 4H), 1.42 (s, 5H), 1.28 (s, 4H). MS (ESI) m/z: 566.1 (M+1)⁺.

Step 2. Synthesis of tert-butyl (S)-2-(6-cyano-8-fluoro-3-(4-methoxyphenyl)-4-oxo-3,4-dihydroquinazolin-2-yl) pyrrolidine-1-carboxylate (16b)

A mixture of 15b (100.00 mg, 0.18 mmol), Copper cyanide (17.95 mg, 0.20 mmol) and tetrakis(triphenylphosphine) palladium (0) (10.40 mg, 0.0090 mmol) in DMF (2 mL) was degassed and flushed with nitrogen. The mixture was heated at 100° C. for 4 h under nitrogen atmosphere. After cooling to room temperature, the mixture was diluted with H₂O (20 mL) and extracted with ethyl acetate (2×40 mL). The combined organic layers were washed with H₂O (40 mL) and brine (40 mL), dried over anhydrous Na₂SO₄, and filtered, then the solvent was evaporated under reduced pressure. The residue was purified by flash chromatography (0% Ethyl acetate/Hexane˜40% Ethyl acetate/Hexane) to afford 16b (56.00 mg, 67.0%) as colorless oil. ¹H NMR (400 MHz, CDCl₃) δ 8.36 (d, J=10.8 Hz, 1H), 7.65 (ddd, J=20.5, 9.0, 1.4 Hz, 1H), 7.51-7.18 (m, 1H), 7.15-7.01 (m, 3H), 4.65-4.56 (m, 1H), 3.91 (d, J=8.6 Hz, 3H), 3.76-3.68 (m, 1H), 3.61-3.41 (m, 1H), 2.18-1.81 (m, 4H), 1.42 (s, 6H), 1.28 (s, 3H). MS (ESI) m/z: 465.1 (M+1)⁺.

Step 3. Synthesis of (S)-8-fluoro-3-(4-methoxyphenyl)-4-oxo-2-(pyrrolidin-2-yl)-3,4-dihydroquinazoline-6-carbonitrile (17b)

Under nitrogen, trifluoroacetic acid (0.16 mL, 2.20 mmol) was added to a solution of 16b (50.00 mg, 0.11 mmol) in dry CH₂Cl₂ (2 mL) at 0° C., then stirred at room temperature overnight, then the solvent was removed in vacuo and the residue was diluted with CH₂Cl₂ (30 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂ to 5% MeOH/CH₂Cl₂) to afford 17b (18.66 mg, 46.4%) as pale yellow solid. ¹H NMR (500 MHz, CDCl₃) δ 8.36 (s, 1H), 7.67 (dd, J=9.1, 1.7 Hz, 1H), 7.23-7.19 (m, 1H), 7.17-7.11 (m, 1H), 7.06 (d, J=9.1 Hz, 2H), 3.89 (s, 3H), 3.31-3.24 (m, 1H), 3.08-3.01 (s, 1H), 2.81-2.76 (m, 1H), 1.86-1.71 (m, 4H). MS (ESI) m/z: 365.1 (M+1)⁺.

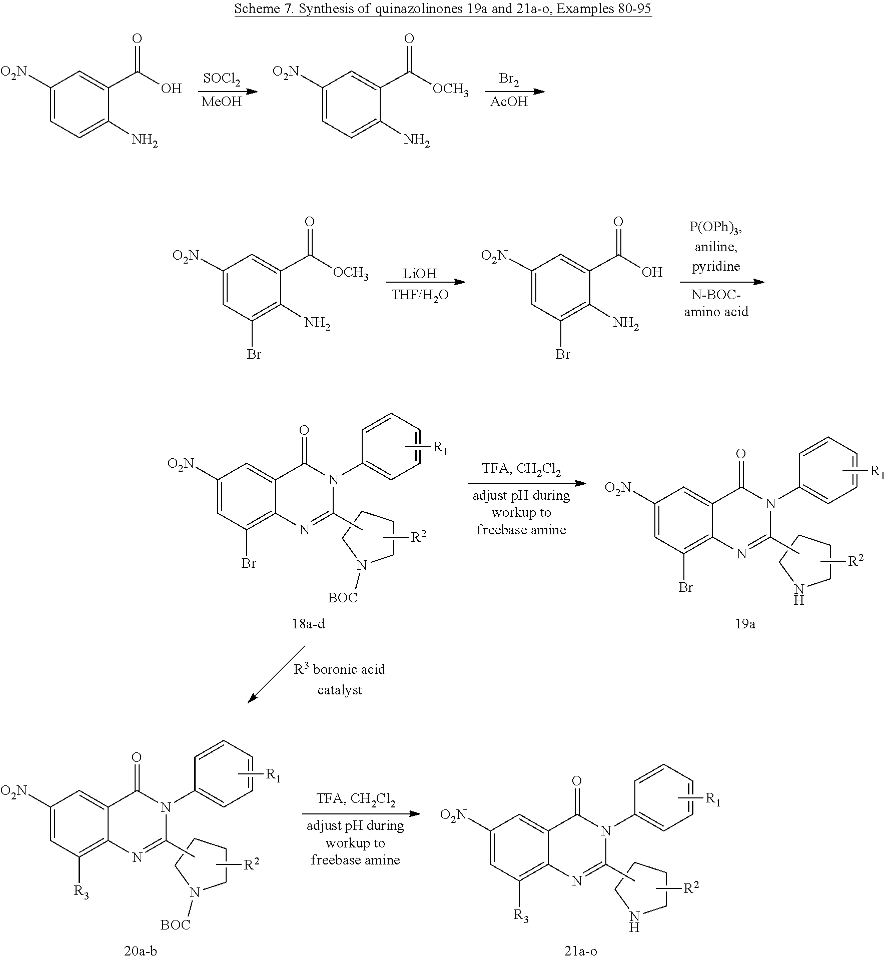

### Example 80. Synthesis of (R)-8-bromo-3-(4-methoxyphenyl)-6-nitro-2-(pyrrolidin-2-yl) quinazolin-4 (3H)-one (19a)

Prepared according to Scheme 7.

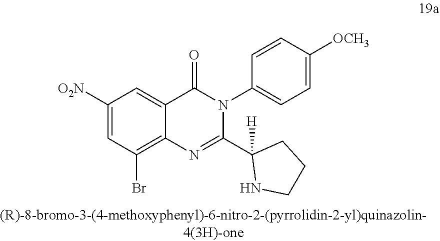 [SMILES: COc1ccc(-n2c([C@H]3CCCN3)nc3c(Br)cc([N+](=O)[O-])cc3c2=O)cc1]

Step 1. Synthesis of methyl 2-amino-5-nitrobenzoate. Under nitrogen, thionyl chloride (8.76 mL, 0.12 mol) was added to a mixture of 5-nitroanthranilic acid (5.46 g, 30.00 mmol) in dry methanol (100 mL) at 0° C., then heated to reflux at room temperature overnight. After cooling down, the reaction was treated with water (100 mL) and extracted with ethyl acetate (3×100 mL). The combined organic layers were washed with saturated NaHCO₃ solution (100 mL), brine (100 mL), dried over anhydrous Na₂SO₄, and filtered, then the solvent was evaporated under reduced pressure to afford methyl 2-amino-5-nitrobenzoate (4.16 g, 70.6%) as pale yellow solid. ¹H NMR (500 MHz, DMSO-d₆) δ 8.61 (d, J=2.8 Hz, 1H), 8.08 (dd, J=9.1 Hz, J=2.8 Hz, 1H), 7.82 (brs, 2H), 6.86 (d, J=9.1 Hz, 1H), 3.86 (s, 3H). MS (ESI) m/z: 197.1 (M+1)⁺.

Step 2. Synthesis of methyl 2-amino-3-bromo-5-nitrobenzoate. Under nitrogen, Bromine (0.12 mL, 2.40 mmol) was added to a reaction mixture of methyl 2-amino-5-nitrobenzoate (392.32 mg, 2.00 mmol) in glacial acetic acid (3 mL) at room temperature, then stirred at room temperature for 4 h, then the mixture was poured into ice water and the resulting precipitate was collected by filtration. After washing the residue with water, the resulting solid was dissolved with acetone, then dried over with anhydrous Na₂SO₄, and filtered, then the solvent was evaporated under reduced pressure to afford methyl 2-amino-3-bromo-5-nitrobenzoate (0.49 g, 89.1%) as pale yellow solid. ¹H NMR (400 MHz, CDCl₃) δ 8.83 (d, J=2.4 Hz, 1H), 8.49 (d, J=2.4 Hz, 1H), 3.95 (s, 3H). MS (ESI) m/z: 276.1 (M+1)⁺.

Step 3. Synthesis of 2-amino-3-bromo-5-nitrobenzoic acid. At 0° C., lithium hydroxide (80.46 mg, 3.36 mmol) was added into the solution of methyl 2-amino-3-bromo-5-nitrobenzoate (0.46 g, 1.68 mmol) in THF (8 mL) and H₂O (8 mL), then stirred at room temperature overnight. The mixture was then adjusted to pH 2 with 1.0N HCl and extracted with ethyl acetate (3×80 mL). The combined organic layers were washed with brine (40 mL) and dried over anhydrous Na₂SO₄, then concentrated under reduced pressure to afford 2-amino-3-bromo-5-nitrobenzoic acid (0.42 g, 95.8%) as pale yellow solid. ¹H NMR (400 MHz, DMSO) δ 13.80 (s, 1H), 8.60 (d, J=2.6 Hz, 1H), 8.43 (d, J=2.6 Hz, 1H). MS (ESI) m/z: 262.1 (M+1)⁺.

Step 4. Synthesis of tert-butyl (R)-2-(8-bromo-3-(4-methoxyphenyl)-6-nitro-4-oxo-3,4-dihydroquinazolin-2-yl) pyrrolidine-1-carboxylate (18a). 2-amino-3-bromo-5-nitrobenzoic acid (65.26 mg, 0.25 mmol) and Boc-D-proline (69.96 mg, 0.33 mmol) were dissolved in dry pyridine (2 mL). Triphenyl phosphite (0.20 mL, 0.75 mmol) was added to the mixture and the reaction mixture was heated at 110° C. with MWI for 40 min. 4-methoxyaniline (36.95 mg, 0.30 mmol) was added to the mixture and the reaction mixture was heated at 100° C. with MWI for 20 min. Pyridine was removed in vacuo and the residue was diluted with CH₂Cl₂ (60 mL) and washed with 1N aqueous HCl (40 mL). The organic layer was washed with H₂O (40 mL) and brine (40 mL), dried over anhydrous Na₂SO₄, and filtered, then the solvent was evaporated under reduced pressure. The residue was purified by flash chromatography (0% Ethyl acetate/Hexane to 40% Ethyl acetate/Hexane) to afford 18a (28.60 mg, 21.0%) as pale yellow solid. ¹H NMR (400 MHz, CDCl₃) δ 9.05 (dd, J=9.6, 2.4 Hz, 1H), 8.81 (dd, J=15.5, 2.4 Hz, 1H), 7.51-7.18 (m, 1H), 7.15-7.01 (m, 3H), 4.58-4.48 (m, 1H), 3.95-3.82 (m, 4H), 3.60-3.41 (m, 1H), 2.30-1.81 (m, 4H), 1.43 (s, 5H), 1.28 (s, 4H). MS (ESI) m/z: 545.1 (M+1)⁺.

Step 5. Synthesis of (R)-8-bromo-3-(4-methoxyphenyl)-6-nitro-2-(pyrrolidin-2-yl) quinazolin-4 (3H)-one (19a)

Under nitrogen, trifluoroacetic acid (0.04 mL, 0.60 mmol) was added to a solution of 18a (16.00 mg, 0.03 mmol) in dry CH₂Cl₂ (1 mL) at 0° C., then stirred at room temperature overnight, then the solvent was removed in vacuo and the residue was diluted with CH₂Cl₂ (10 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 19a (10.60 mg, 73.5%) as pale yellow solid. ¹H NMR (500 MHz, CDCl₃) δ 9.06 (d, J=2.5 Hz, 1H), 8.83 (d, J=2.5 Hz, 1H), 7.23-7.19 (m, 1H), 7.18-7.15 (m, 1H), 7.09 (s, 1H), 7.07 (d, J=1.3 Hz, 1H), 3.95 (t, J=7.1 Hz, 1H), 3.89 (s, 3H), 3.39-3.30 (m, 1H), 2.92-2.84 (m, 1H), 1.92-1.75 (m, 4H). MS (ESI) m/z: 445.1 (M+1)⁺.

### Example 81. Synthesis of (R)-3-(3-fluoro-4-methoxyphenyl)-6-nitro-8-(pyridin-3-yl)-2-(pyrrolidin-2-yl) quinazolin-4 (3H)-one (21a)

Prepared according to Scheme 7.

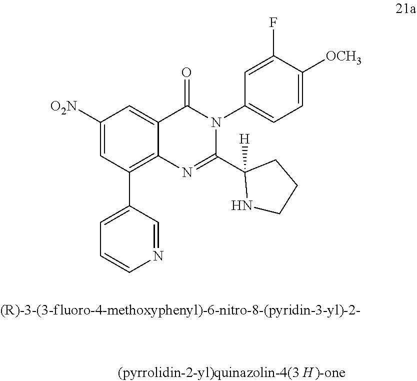 [SMILES: COc1ccc(-n2c([C@H]3CCCN3)nc3c(-c4cccnc4)cc([N+](=O)[O-])cc3c2=O)cc1F]

Step 1. Synthesis of tert-butyl (R)-2-(8-bromo-3-(3-fluoro-4-methoxyphenyl)-6-nitro-4-oxo-3,4-dihydroquin-azolin-2-yl) pyrrolidine-1-carboxylate (18b). 2-amino-3-bromo-5-nitrobenzoic acid (261.06 mg, 1.00 mmol) and Boc-D-proline (280.00 mg, 1.30 mmol) were dissolved in dry pyridine (5 mL). Triphenyl phosphite (0.79 mL, 3.00 mmol) was added to the mixture and the reaction mixture was heated at 110° C. with MWI for 40 min. 3-Fluoro-4-methoxyaniline (169.75 mg, 1.20 mmol) was added to the mixture and the reaction mixture was heated at 100° C. with MWI for 30 min. Pyridine was removed in vacuo and the residue was diluted with CH₂Cl₂ (100 mL) and washed with 1N aqueous HCl (50 mL). The organic layer was washed with H₂O (50 mL) and brine (50 mL), dried over anhydrous Na₂SO₄, and filtered, then the solvent was evaporated under reduced pressure. The residue was purified by flash chromatography (0% Ethyl acetate/Hexane˜40% Ethyl acetate/Hexane) to afford 18b (0.14 g, 24.9%) as pale yellow solid. ¹H NMR (400 MHz, CDCl₃) δ 9.06-9.00 (m, 1H), 8.81 (dd, J=16.4, 1.9 Hz, 1H), 7.45-7.08 (m, 2H), 7.05-6.91 (m, 1H), 4.62-4.50 (m, 1H), 4.01-3.96 (m, 3H), 3.91-3.84 (m, 1H), 3.62-3.45 (m, 1H), 2.28-1.87 (m, 4H), 1.43 (d, J=4.2 Hz, 6H), 1.29 (d, J=1.6 Hz, 3H). MS (ESI) m/z: 563.1 (M+1)⁺.

Step 2. Synthesis of (R)-3-(3-fluoro-4-methoxyphenyl)-6-nitro-8-(pyridin-3-yl)-2-(pyrrolidin-2-yl) quinazolin-4 (3H)-one (21a)

A mixture of 18b (30.00 mg, 0.050 mmol), cesium carbonate (32.58 mg, 0.10 mmol), pyridine-3-boronic acid (9.26 mg, 0.075 mmol) and tetrakis(triphenylphosphine) palladium (0) (6.00 mg, 0.005 mmol) in dioxane (1.5 mL) and H₂O (0.6 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.05 mL, 0.80 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (30 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 21a (6.20 mg, 26.9%) as pale yellow solid. ¹H NMR (500 MHz, CDCl₃) δ 9.16 (d, J=2.6 Hz, 1H), 8.92 (d, J=2.2 Hz, 1H), 8.71 (d, J=4.2 Hz, 1H), 8.63 (d, J=2.6 Hz, 1H), 8.00-7.98 (m, 1H), 7.47 (dd, J=7.6, 5.1 Hz, 1H), 7.18-7.00 (m, 3H), 3.99 (d, J=0.7 Hz, 3H), 3.91 (dd, J=14.2, 7.9 Hz, 1H), 3.11-3.01 (m, 1H), 2.80-2.72 (m, 1H), 1.83-1.65 (m, 4H). MS (ESI) m/z: 462.1 (M+1)⁺.

### Example 82. Synthesis of (R)-3-(3-fluoro-4-methoxyphenyl)-6-nitro-8-(pyridin-4-yl)-2-(pyrrolidin-2-yl) quinazolin-4 (3H)-one (21b)

Prepared according to Scheme 7.

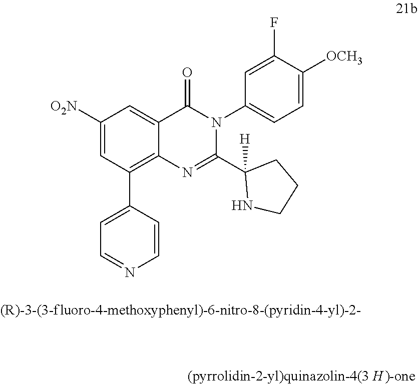 [SMILES: COc1ccc(-n2c([C@H]3CCCN3)nc3c(-c4ccncc4)cc([N+](=O)[O-])cc3c2=O)cc1F]

### Synthesis of (R)-3-(3-fluoro-4-methoxyphenyl)-6-nitro-8-(pyridin-4-yl)-2-(pyrrolidin-2-yl) quinazolin-4 (3H)-one (21b)

A mixture of 18b (30.00 mg, 0.050 mmol), cesium carbonate (32.58 mg, 0.10 mmol), pyridine-4-boronic acid (9.26 mg, 0.075 mmol) and tetrakis(triphenylphosphine) palladium (0) (6.00 mg, 0.005 mmol) in dioxane (1.5 mL) and H₂O (0.6 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.05 mL, 0.80 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (30 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂₅% MeOH/CH₂Cl₂) to afford 21b (6.80 mg, 29.5%) as pale yellow solid. ¹H NMR (400 MHz, CDCl₃) δ 9.17 (d, J=2.4 Hz, 1H), 8.78 (d, J=5.4 Hz, 2H), 8.63 (d, J=2.3 Hz, 1H), 7.60 (d, J=5.6 Hz, 2H), 7.18-6.96 (m, 3H), 3.99 (s, 3H), 3.96-3.86 (m, 1H), 3.08-3.01 (m, 1H), 2.81-2.75 (m, 1H), 2.40 (brs, 1H), 1.81-1.65 (m, 4H). MS (ESI) m/z: 462.1 (M+1)⁺.

### Example 83. Synthesis of (S)-3-(3-fluoro-4-methoxyphenyl)-6-nitro-8-(pyridin-3-yl)-2-(pyrrolidin-2-yl) quinazolin-4 (3H)-one (21c)

Prepared according to Scheme 7.

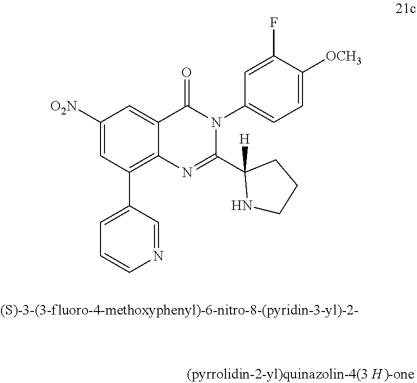 [SMILES: COc1ccc(-n2c([C@@H]3CCCN3)nc3c(-c4cccnc4)cc([N+](=O)[O-])cc3c2=O)cc1F]

Step 1. Synthesis of tert-butyl (S)-2-(8-bromo-3-(3-fluoro-4-methoxyphenyl)-6-nitro-4-oxo-3,4-dihydroquin-azolin-2-yl) pyrrolidine-1-carboxylate (18c). 2-amino-3-bromo-5-nitrobenzoic acid (261.06 mg, 1.00 mmol) and Boc-L-proline (280.00 mg, 1.30 mmol) were dissolved in dry pyridine (5 mL). Triphenyl phosphite (0.79 mL, 3.00 mmol) was added to the mixture and the reaction mixture was heated at 110° C. with MWI for 40 min. 3-Fluoro-4-methoxyaniline (169.75 mg, 1.20 mmol) was added to the mixture and the reaction mixture was heated at 100° C. with MWI for 30 min. Pyridine was removed in vacuo and the residue was diluted with CH₂Cl₂ (100 mL) and washed with 1N HCl aqueous (50 mL). The organic layer was washed with H₂O (50 mL) and brine (50 mL), dried over anhydrous Na₂SO₄, and filtered, then the solvent was evaporated under reduced pressure. The residue was purified by flash chromatography (0% Ethyl acetate/Hexane˜40% Ethyl acetate/Hexane) to afford 18c (0.15 g, 26.7%) as pale yellow solid. ¹H NMR (400 MHz, CDCl₃) δ 9.05-9.88 (m, 1H), 8.81 (dd, J=16.5, 1.9 Hz, 1H), 7.45-7.08 (m, 2H), 7.06-6.88 (m, 1H), 4.61-4.48 (m, 1H), 4.01-3.95 (m, 3H), 3.91-3.85 (m, 1H), 3.61-3.45 (m, 1H), 2.31-1.86 (m, 4H), 1.43 (d, J=4.2 Hz, 6H), 1.28 (d, J=1.6 Hz, 3H). MS (ESI) m/z: 563.1 (M+1)⁺.

Step 2. Synthesis of (S)-3-(3-fluoro-4-methoxyphenyl)-6-nitro-8-(pyridin-3-yl)-2-(pyrrolidin-2-yl) quinazolin-4 (3H)-one (21c)

A mixture of 18c (30.00 mg, 0.050 mmol), cesium carbonate (32.58 mg, 0.10 mmol), pyridine-3-boronic acid (9.26 mg, 0.075 mmol) and tetrakis(triphenylphosphine) palladium (0) (6.00 mg, 0.005 mmol) in dioxane (1.5 mL) and H₂O (0.6 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.05 mL, 0.80 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (30 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 21c (9.60 mg, 41.6%) as pale yellow solid. ¹H NMR (500 MHz, CDCl₃) δ 9.16 (d, J=2.6 Hz, 1H), 8.92 (d, J=2.2 Hz, 1H), 8.73-8.70 (m, 1H), 8.63 (dd, J=2.6, 0.6 Hz, 1H), 7.99 (d, J=7.9 Hz, 1H), 7.48-7.45 (m, 1H), 7.16-7.00 (m, 3H), 3.99 (d, J=0.7 Hz, 3H), 3.98-3.86 (m, 1H), 3.08-3.01 (m, 1H), 2.81-2.75 (m, 1H), 1.86-1.65 (m, 4H). MS (ESI) m/z: 462.1 (M+1)⁺.

### Example 84. Synthesis of (S)-3-(3-fluoro-4-methoxyphenyl)-6-nitro-8-(pyridin-4-yl)-2-(pyrrolidin-2-yl) quinazolin-4 (3H)-one (21d)

Prepared according to Scheme 7.

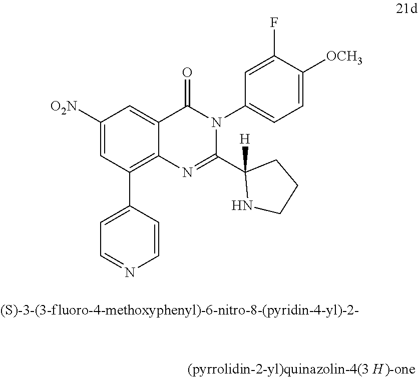 [SMILES: COc1ccc(-n2c([C@@H]3CCCN3)nc3c(-c4ccncc4)cc([N+](=O)[O-])cc3c2=O)cc1F]

### Synthesis of (S)-3-(3-fluoro-4-methoxyphenyl)-6-nitro-8-(pyridin-4-yl)-2-(pyrrolidin-2-yl) quinazolin-4 (3H)-one (21d)

A mixture of 18c (30.00 mg, 0.050 mmol), cesium carbonate (32.58 mg, 0.10 mmol), pyridine-4-boronic acid (9.26 mg, 0.075 mmol) and tetrakis(triphenylphosphine) palladium (0) (6.00 mg, 0.005 mmol) in dioxane (1.5 mL) and H₂O (0.6 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step. Under nitrogen, trifluoroacetic acid (0.05 mL, 0.80 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (30 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 21d (11.50 mg, 49.8%) as pale yellow solid. ¹H NMR (400 MHz, CDCl₃) δ 9.16 (d, J=2.4 Hz, 1H), 8.76 (d, J=5.4 Hz, 2H), 8.61 (d, J=2.3 Hz, 1H), 7.61 (d, J=5.6 Hz, 2H), 7.21-6.96 (m, 3H), 3.98 (s, 3H), 3.96-3.85 (m, 1H), 3.11-3.01 (m, 1H), 2.81-2.75 (m, 1H), 2.41 (brs, 1H), 1.81-1.65 (m, 4H). MS (ESI) m/z: 462.1 (M+1)⁺.

### Example 85. Synthesis of (R)-3-(4-methoxyphenyl)-6-nitro-8-(pyridin-3-yl)-2-(pyrrolidin-2-yl) quinazolin-4 (3H)-one (21e)

Prepared according to Scheme 7.

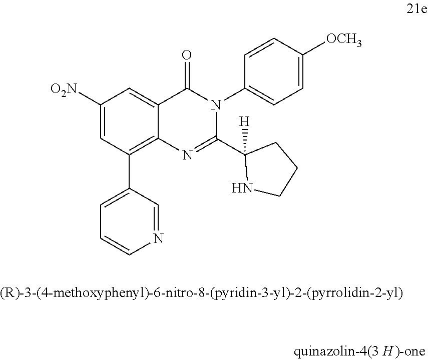 [SMILES: COc1ccc(-n2c([C@H]3CCCN3)nc3c(-c4cccnc4)cc([N+](=O)[O-])cc3c2=O)cc1]

Step 1. Synthesis of tert-butyl (R)-2-(3-(4-methoxyphenyl)-6-nitro-4-oxo-8-(pyridin-3-yl)-3,4-dihydroquinazol-in-2-yl) pyrrolidine-1-carboxylate (20a)

A mixture of 18a (20.00 mg, 0.036 mmol), cesium carbonate (23.49 mg, 0.072 mmol), pyridine-3-boronic acid (6.76 mg, 0.055 mmol) and tetrakis(triphenylphosphine) palladium (0) (4.16 mg, 0.0036 mmol) in dioxane (1 mL) and H₂O (0.5 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure. The residue was purified by flash chromatography (0% MeOH/CH₂Cl₂˜ 5% MeOH/CH₂Cl₂) to afford 20a (16.86 mg, 86.2%) as pale yellow solid. The crude product was treated with a palladium scavenger at elevated temperature to remove any residual metal from the coupling reaction. The crude product was separated from the scavenger by filtration and used in the next step. ¹H NMR (500 MHz, CDCl₃) δ 9.16 (dd, J=8.9, 2.6 Hz, 1H), 8.86 (d, J=44.7 Hz, 1H), 8.70 (d, J=3.5 Hz, 1H), 8.63 (dd, J=24.5, 2.6 Hz, 1H), 8.07 (dd, J=28.9, 7.9 Hz, 1H), 7.71-7.64 (m, 1H), 7.52-7.38 (m, 2H), 7.16-7.10 (m, 1H), 7.15-7.06 (m, 1H), 4.56-4.45 (m, 1H), 3.89 (d, J=10.9 Hz, 2H), 3.70 (s, 1H), 3.45-3.32 (m, 2H), 2.00-1.70 (m, 4H), 1.33 (s, 5H), 1.28 (s, 4H). MS (ESI) m/z: 544.1 (M+1)⁺.

Step 2. Synthesis of (R)-3-(4-methoxyphenyl)-6-nitro-8-(pyridin-3-yl)-2-(pyrrolidin-2-yl) quinazolin-4 (3H)-one (21e)

Under nitrogen, trifluoroacetic acid (0.04 mL, 0.60 mmol) was added to a solution of 20a (16.00 mg, 0.03 mmol) in dry CH₂Cl₂ (1 mL) at 0° C., then stirred at room temperature overnight, then the solvent was removed in vacuo and the residue was diluted with CH₂Cl₂ (10 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 21e (6.20 mg, 46.6%) as pale yellow solid. ¹H NMR (500 MHz, CDCl₃) δ 9.17 (d, J=2.7 Hz, 1H), 8.93 (d, J=1.7 Hz, 1H), 8.71 (dd, J=4.8, 1.6 Hz, 1H), 8.62 (d, J=2.7 Hz, 1H), 8.08-7.96 (m, 1H), 7.50-7.44 (m, 1H), 7.25-7.20 (m, 1H), 7.20-7.16 (m, 1H), 7.08 (d, J=9.1 Hz, 2H), 3.97-3.91 (m, 1H), 3.90 (s, 3H), 3.11-3.03 (m, 1H), 2.78-2.68 (m, 1H), 2.09 (brs, 1H), 1.81-1.68 (m, 4H). MS (ESI) m/z: 444.1 (M+1)⁺.

### Example 86. Synthesis of 2-((2R,4S)-4-fluoropyrrolidin-2-yl)-3-(4-methoxyphenyl)-6-nitro-8-(pyridin-3-yl) quinazolin-4 (3H)-one (21f)

Prepared according to Scheme 7.

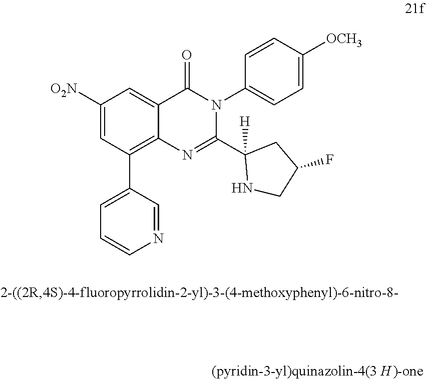 [SMILES: COc1ccc(-n2c([C@H]3C[C@H](F)CN3)nc3c(-c4cccnc4)cc([N+](=O)[O-])cc3c2=O)cc1]

Step 1. Synthesis of tert-butyl (2R,4S)-2-(8-bromo-3-(4-methoxyphenyl)-6-nitro-4-oxo-3,4-dihydroquinazolin-2-yl)-4-fluoropyrrolidine-1-carboxylate (18d). 2-amino-3-bromo-5-nitrobenzoic acid (65.26 mg, 0.25 mmol) and (2R,4S)-1-(tert-butoxycarbonyl)-4-fluoropyrrolidine-2-carboxylic acid (75.80 mg, 0.33 mmol) were dissolved in dry pyridine (2 mL). Triphenyl phosphite (0.20 mL, 0.75 mmol) was added to the mixture and the reaction mixture was heated at 100° C. with MWI for 40 min. 4-methoxyaniline (36.95 mg, 0.30 mmol) was added to the mixture and the reaction mixture was heated at 120° C. with MWI for 1×30 min. Pyridine was removed in vacuo and the residue was diluted with CH₂Cl₂ (40 mL) and washed with 1N HCl aqueous (30 mL). The organic layer was washed with H₂O (30 mL) and brine (30 mL), dried over anhydrous Na₂SO₄, and filtered, then the solvent was evaporated under reduced pressure. The residue was purified by flash chromatography (0% Ethyl acetate/Hexane˜40% Ethyl acetate/Hexane) to afford 18d (19.80 mg, 14.1%) as pale yellow solid. ¹H NMR (400 MHz, CDCl₃) δ 9.04 (dd, J=11.9, 2.4 Hz, 1H), 8.81 (dd, J=17.7, 2.4 Hz, 1H), 7.56-7.21 (m, 1H), 7.181-7.05 (m, 3H), 5.28 (dd, J=52.9, 24.0 Hz, 1H), 4.81-4.68 (m, 1H), 4.06-3.84 (m, 5H), 2.48-2.18 (m, 2H), 1.42 (s, 6H), 1.26 (s, 3H). MS (ESI) m/z: 563.1 (M+1)⁺.

Step 2. Synthesis of 2-((2R,4S)-4-fluoropyrrolidin-2-yl)-3-(4-methoxyphenyl)-6-nitro-8-(pyridin-3-yl) quinaz-olin-4 (3H)-one (21f)

A mixture of 18d (19.80 mg, 0.035 mmol), cesium carbonate (22.86 mg, 0.07 mmol), pyridine-3-boronic acid (6.80 mg, 0.055 mmol) and tetrakis(triphenylphosphine) palladium (0) (4.00 mg, 0.0035 mmol) in dioxane (1.5 mL) and H₂O (0.6 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.04 mL, 0.50 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (30 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 21f (6.60 mg, 40.9%) as pale yellow amorphous solid. ¹H NMR (500 MHz, CDCl₃) δ 9.17 (d, J=2.6 Hz, 1H), 8.91 (d, J=2.1 Hz, 1H), 8.70 (dd, J=4.8, 1.5 Hz, 1H), 8.62 (d, J=2.6 Hz, 1H), 7.98-7.94 (m, 1H), 7.46 (dd, J=7.9, 4.9 Hz, 1H), 7.33-7.29 (m, 1H), 7.18-7.14 (m, 1H), 7.11-7.06 (m, 2H), 5.18 (d, J=54.7 Hz, 1H), 4.23 (t, J=7.6 Hz, 1H), 3.90 (s, 3H), 3.11-3.08 (m, 1H), 3.06-3.04 (m, 1H), 2.25-2.12 (m, 2H). MS (ESI) m/z: 462.1 (M+1)⁺.

### Example 87. Synthesis of (R)-3-(4-methoxyphenyl)-6-nitro-8-(pyrimidin-5-yl)-2-(pyrrolidin-2-yl) quinazolin-4 (3H)-one (21g)

Prepared according to Scheme 7.

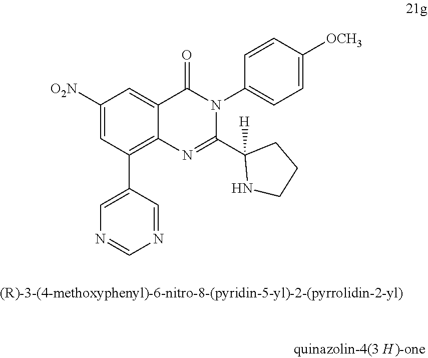 [SMILES: COc1ccc(-n2c([C@H]3CCCN3)nc3c(-c4cncnc4)cc([N+](=O)[O-])cc3c2=O)cc1]

### Synthesis of (R)-3-(4-methoxyphenyl)-6-nitro-8-(pyrimidin-5-yl)-2-(pyrrolidin-2-yl) quinazolin-4 (3H)-one (21g)

A mixture of 18a (40.00 mg, 0.075 mmol), cesium carbonate (48.86 mg, 0.15 mmol), 5-pyrimidinylboronic acid (13.65 mg, 0.11 mmol) and tetrakis(triphenylphosphine) palladium (0) (8.66 mg, 0.0075 mmol) in dioxane (2.5 mL) and H₂O (1 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×30 mL). The combined organic layers were washed with H₂O (10 mL) and brine (10 mL), dried over anhydrous Na₂SO₄, and filtered, then the solvent was evaporated under reduced pressure to afford pale yellow oil, which was used for the following reaction without further purification. The crude product was treated with a palladium scavenger at elevated temperature to remove any residual metal from the coupling reaction. The crude product was separated from the scavenger by filtration and used in the next step.

Under nitrogen, trifluoroacetic acid (0.05 mL, 0.80 mmol) was added to a solution of pale yellow oil described above in dry CH₂Cl₂ (1.5 mL) at 0° C., then stirred at room temperature overnight, then the solvent was removed in vacuo and the residue was diluted with CH₂Cl₂ (30 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 21g (8.60 mg, 25.8%) as pale yellow solid. ¹H NMR (500 MHz, CDCl₃) δ 9.31 (s, 1H), 9.19 (d, J=2.4 Hz, 1H), 9.09 (s, 2H), 8.64 (d, J=2.4 Hz, 1H), 7.28-7.25 (m, 1H), 7.18-7.16 (m, 1H), 7.08 (d, J=8.8 Hz, 2H), 4.02-3.97 (m, 1H), 3.90 (s, 3H), 3.15-3.08 (m, 1H), 2.86-2.78 (m, 1H), 2.16 (brs, 1H), 1.86-1.68 (m, 4H). MS (ESI) m/z: 445.1 (M+1)⁺.

### Example 88. Synthesis of (R)-3-(3-fluoro-4-methoxyphenyl)-6-nitro-8-(pyrimidin-5-yl)-2-(pyrrolidin-2-yl) quinazol-in-4 (3H)-one (21h)

Prepared according to Scheme 7.

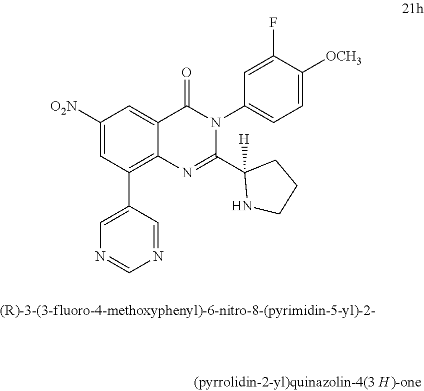 [SMILES: COc1ccc(-n2c([C@H]3CCCN3)nc3c(-c4cncnc4)cc([N+](=O)[O-])cc3c2=O)cc1F]

### Synthesis of (R)-3-(3-fluoro-4-methoxyphenyl)-6-nitro-8-(pyrimidin-5-yl)-2-(pyrrolidin-2-yl) quinazolin-4 (3H)-one (21h)

A mixture of 18b (42.25 mg, 0.075 mmol), cesium carbonate (48.86 mg, 0.15 mmol), 5-pyrimidinylboronic acid (13.65 mg, 0.11 mmol) and tetrakis(triphenylphosphine) palladium (0) (8.66 mg, 0.0075 mmol) in dioxane (2.5 mL) and H₂O (1 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.05 mL, 0.80 mmol) was added to a solution of pale yellow oil described above in dry CH₂Cl₂ (1.5 mL) at 0° C., then stirred at room temperature overnight, then the solvent was removed in vacuo and the residue was diluted with CH₂Cl₂ (40 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 21 h (9.60 mg, 27.8%) as pale yellow solid. ¹H NMR (500 MHz, CDCl₃) δ 9.32 (s, 1H), 9.20 (d, J=2.6 Hz, 1H), 9.08 (s, 2H), 8.65 (d, J=2.6 Hz, 1H), 7.18-7.08 (m, 2H), 7.06-7.01 (m, 1H), 4.10-3.86 (m, 4H), 3.11-3.06 (m, 1H), 2.85-2.75 (m, 1H), 1.88-1.65 (m, 4H). MS (ESI) m/z: 463.1 (M+1)⁺.

### Example 89. Synthesis of (R)-3-(3-fluoro-4-methoxyphenyl)-8-(6-fluoropyridin-3-yl)-6-nitro-2-(pyrrolidin-2-yl) quinazolin-4 (3H)-one (21i)

Prepared according to Scheme 7.

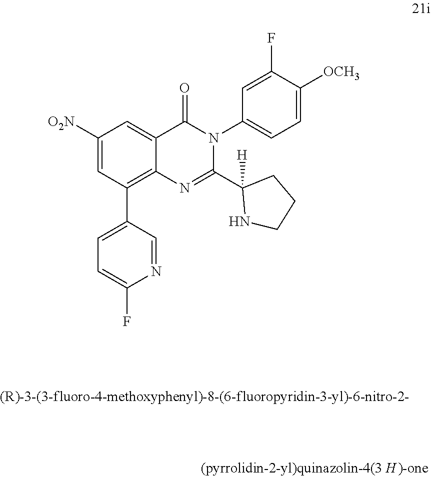 [SMILES: COc1ccc(-n2c([C@H]3CCCN3)nc3c(-c4ccc(F)nc4)cc([N+](=O)[O-])cc3c2=O)cc1F]

### Synthesis of (R)-3-(3-fluoro-4-methoxyphenyl)-8-(6-fluoropyridin-3-yl)-6-nitro-2-(pyrrolidin-2-yl) quinazolin-4 (3H)-one (21i)

A mixture of 18b (42.25 mg, 0.075 mmol), cesium carbonate (48.86 mg, 0.15 mmol), 2-fluoropyridine-5-boronic acid (15.50 mg, 0.11 mmol) and tetrakis(tri-phenylphosphine) palladium (0) (8.66 mg, 0.0075 mmol) in dioxane (3 mL) and H₂O (1 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.05 mL, 0.80 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (40 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 21i (10.91 mg, 30.5%) as pale yellow solid. ¹H NMR (500 MHz, CDCl₃) δ 9.15 (d, J=2.6 Hz, 1H), 8.61 (d, J=2.6 Hz, 1H), 8.54 (d, J=2.0 Hz, 1H), 8.12 (td, J=7.7, 1.9 Hz, 1H), 7.18-6.98 (m, 4H), 3.99 (s, 3H), 3.96-3.86 (m, 1H), 3.11-3.06 (m, 1H), 2.81-2.75 (m, 1H), 2.31 (brs, 1H), 1.86-1.65 (m, 4H). MS (ESI) m/z: 480.1 (M+1)⁺.

### Example 90. Synthesis of (R)-3-(3-fluoro-4-methoxyphenyl)-8-(2-fluoropyridin-4-yl)-6-nitro-2-(pyrrolidin-2-yl) quinazolin-4 (3H)-one (21j)

Prepared according to Scheme 7.

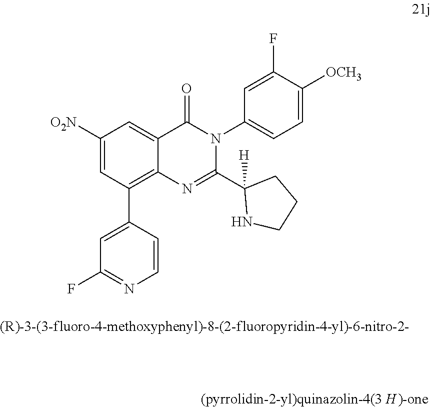 [SMILES: COc1ccc(-n2c([C@H]3CCCN3)nc3c(-c4ccnc(F)c4)cc([N+](=O)[O-])cc3c2=O)cc1F]

### Synthesis of (R)-3-(3-fluoro-4-methoxyphenyl)-8-(2-fluoropyridin-4-yl)-6-nitro-2-(pyrrolidin-2-yl) quinazolin-4 (3H)-one (21j)

A mixture of 18b (42.25 mg, 0.075 mmol), cesium carbonate (48.86 mg, 0.15 mmol), 2-fluoropyridine-4-boronic acid (15.50 mg, 0.11 mmol) and tetrakis(tri-phenylphosphine) palladium (0) (8.66 mg, 0.0075 mmol) in dioxane (3 mL) and H₂O (1 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.05 mL, 0.80 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (40 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 21j (11.16 mg, 31.1%) as pale yellow solid. ¹H NMR (500 MHz, CDCl₃) δ 9.19 (d, J=2.6 Hz, 1H), 8.64 (d, J=2.6 Hz, 1H), 8.38 (d, J=5.1 Hz, 1H), 7.49 (d, J=5.2 Hz, 1H), 7.29 (s, 1H), 7.18-7.11 (m, 1H), 7.10-7.06 (m, 1H), 7.05-7.01 (m, 1H), 4.02-3.91 (m, 4H), 3.13-3.03 (m, 1H), 2.81-2.75 (m, 1H), 2.21 (brs, 1H), 1.86-1.68 (m, 4H). MS (ESI) m/z: 480.1 (M+1)⁺.

### Example 91. Synthesis of (R)-3-(3-fluoro-4-methoxyphenyl)-6-nitro-2-(pyrrolidin-2-yl)-8-(6-(trifluoromethyl)pyridin-3-yl) quinazolin-4 (3H)-one (21k)

Prepared according to Scheme 7.

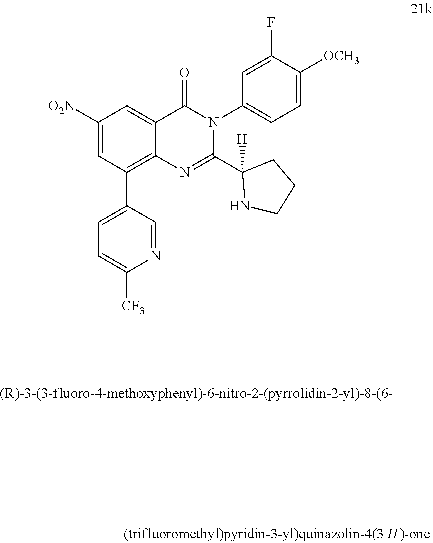 [SMILES: COc1ccc(-n2c([C@H]3CCCN3)nc3c(-c4ccc(C(F)(F)F)nc4)cc([N+](=O)[O-])cc3c2=O)cc1F]

### Synthesis of (R)-3-(3-fluoro-4-methoxyphenyl)-6-nitro-2-(pyrrolidin-2-yl)-8-(6-(trifluoromethyl)pyridin-3-yl) quinazolin-4 (3H)-one (21k)

A mixture of 18b (42.25 mg, 0.075 mmol), cesium carbonate (48.86 mg, 0.15 mmol), 2-trifluoromethyl-5-pyridineboric acid (21.00 mg, 0.11 mmol) and tetrakis(triphenylphosphine) palladium (0) (8.66 mg, 0.0075 mmol) in dioxane (3 mL) and H₂O (1 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.05 mL, 0.80 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (40 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 21k (7.25 mg, 18.3%) as pale yellow solid. ¹H NMR (500 MHz, CDCl₃) δ 9.19 (d, J=2.6 Hz, 1H), 9.04 (s, 1H), 8.65 (d, J=2.5 Hz, 1H), 8.18 (dd, J=8.0, 1.5 Hz, 1H), 7.86 (d, J=8.1 Hz, 1H), 7.18-7.01 (m, 4H), 4.06-3.86 (m, 4H), 3.08-3.01 (m, 1H), 2.81-2.75 (m, 1H), 2.18 (s, 2H), 1.88-1.65 (m, 4H). MS (ESI) m/z: 530.1 (M+1)⁺.

### Example 92. Synthesis of (R)-3-(3-fluoro-4-methoxyphenyl)-8-(6-methylpyridin-3-yl)-6-nitro-2-(pyrrolidin-2-yl) quinazolin-4 (3H)-one (21l)

Prepared according to Scheme 7.

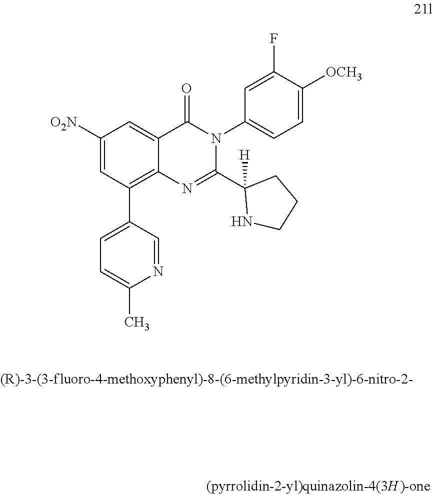 [SMILES: COc1ccc(-n2c([C@H]3CCCN3)nc3c(-c4ccc(C)nc4)cc([N+](=O)[O-])cc3c2=O)cc1F]

### Synthesis of (R)-3-(3-fluoro-4-methoxyphenyl)-8-(6-methylpyridin-3-yl)-6-nitro-2-(pyrrolidin-2-yl) quinazolin-4 (3H)-one (21l)

A mixture of 18b (42.25 mg, 0.075 mmol), cesium carbonate (48.86 mg, 0.15 mmol), 6-methylpyridine-3-boronic acid (15.06 mg, 0.11 mmol) and tetrakis(triphenylphosphine) palladium (0) (8.66 mg, 0.0075 mmol) in dioxane (3 mL) and H₂O (1 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.05 mL, 0.80 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (40 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 21l (7.28 mg, 20.5%) as pale yellow solid. ¹H NMR (500 MHz, CDCl₃) δ 9.13 (d, J=2.6 Hz, 1H), 8.80 (d, J=2.1 Hz, 1H), 8.60 (d, J=2.6 Hz, 1H), 7.89 (dd, J=8.0, 2.3 Hz, 1H), 7.32 (d, J=8.0 Hz, 1H), 7.16-7.11 (m, 1H), 7.08-7.01 (m, 2H), 3.99 (d, J=0.9 Hz, 3H), 3.94-3.86 (m, 1H), 3.13-3.05 (m, 1H), 2.81-2.75 (m, 1H), 2.69-2.65 (m, 3H), 1.81-1.68 (m, 4H). MS (ESI) m/z: 476.1 (M+1)⁺.

### Example 93. Synthesis of (R)-3-(3-fluoro-4-methoxyphenyl)-8-(5-fluoropyridin-3-yl)-6-nitro-2-(pyrrolidin-2-yl) quinazolin-4 (3H)-one (21m)

Prepared according to Scheme 7.

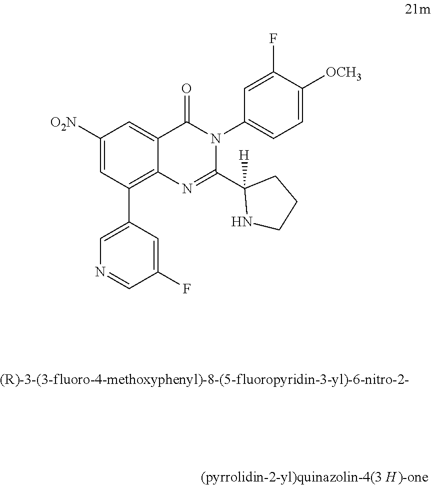 [SMILES: COc1ccc(-n2c([C@H]3CCCN3)nc3c(-c4cncc(F)c4)cc([N+](=O)[O-])cc3c2=O)cc1F]

### Synthesis of (R)-3-(3-fluoro-4-methoxyphenyl)-8-(5-fluoropyridin-3-yl)-6-nitro-2-(pyrrolidin-2-yl) quinazolin-4 (3H)-one (21m)

A mixture of 18b (42.25 mg, 0.075 mmol), cesium carbonate (48.86 mg, 0.15 mmol), 5-fluoropyridin-3-ylboronic acid (15.50 mg, 0.11 mmol) and tetrakis(triphenylphosphine) palladium (0) (8.66 mg, 0.0075 mmol) in dioxane (3 mL) and H₂O (1 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.05 mL, 0.80 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (40 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 21m (14.10 mg, 39.2%) as pale yellow solid. ¹H NMR (500 MHz, CDCl₃) δ 9.17 (d, J=2.6 Hz, 1H), 8.73 (s, 1H), 8.65 (d, J=2.6 Hz, 1H), 8.58 (d, J=2.6 Hz, 1H), 7.82-7.75 (m, 1H), 7.18-7.11 (m, 1H), 7.10-7.06 (m, 1H), 7.05-7.01 (m, 1H), 3.99 (s, 3H), 3.93 (dd, J=13.8, 6.5 Hz, 1H), 3.11-3.05 (m, 1H), 2.81-2.75 (m, 1H), 2.18 (brs, 1H), 1.85-1.75 (m, 4H). MS (ESI) m/z: 480.1 (M+1)⁺.

### Example 94. Synthesis of (R)-3-(3-fluoro-4-methoxyphenyl)-8-(5-cyanopyridin-3-yl)-6-nitro-2-(pyrrolidin-2-yl) quinazolin-4 (3H)-one (21n)

Prepared according to Scheme 7.

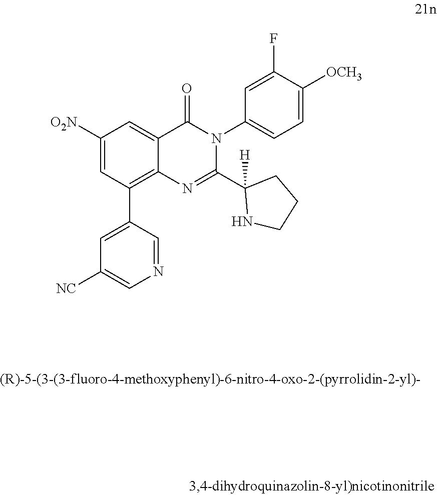 [SMILES: COc1ccc(-n2c([C@H]3CCCN3)nc3c(-c4cncc(C#N)c4)cc([N+](=O)[O-])cc3c2=O)cc1F]

### Synthesis of (R)-3-(3-fluoro-4-methoxyphenyl)-8-(5-cyanopyridin-3-yl)-6-nitro-2-(pyrrolidin-2-yl) quinazolin-4 (3H)-one (21n)

A mixture of 18b (42.25 mg, 0.075 mmol), cesium carbonate (48.86 mg, 0.15 mmol), 5-cyanopyridine-3-boronic acid (16.28 mg, 0.11 mmol) and tetrakis(tri-phenylphosphine) palladium (0) (8.66 mg, 0.0075 mmol) in dioxane (3 mL) and H₂O (1 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.06 mL, 0.86 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (40 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 21n (13.60 mg, 37.5%) as pale yellow solid. ¹H NMR (500 MHz, CDCl₃) δ 9.19 (d, J=2.6 Hz, 1H), 9.11 (d, J=2.2 Hz, 1H), 8.98 (d, J=1.8 Hz, 1H), 8.65 (d, J=2.6 Hz, 1H), 8.37 (t, J=1.9 Hz, 1H), 7.18-7.11 (m, 1H), 7.12-7.07 (m, 1H), 7.06-7.01 (m, 1H), 4.00-3.93 (m, 4H), 3.08-3.04 (m, 1H), 2.86-2.82 (m, 1H), 2.06 (s, 1H), 1.86-1.71 (m, 4H). MS (ESI) m/z: 487.1 (M+1)⁺.

### Example 95. Synthesis of (R)-8-cyclopropyl-3-(4-methoxyphenyl)-6-nitro-2-(pyrrolidin-2-yl) quinazolin-4 (3H)-one (210)

Prepared according to Scheme 7.

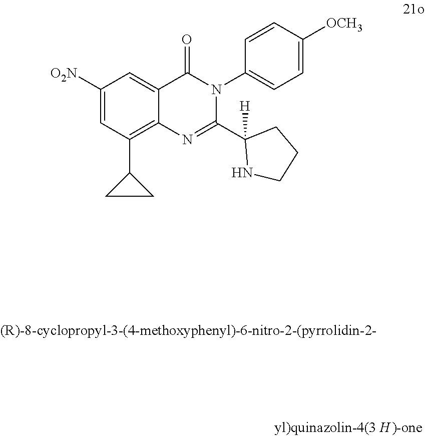 [SMILES: COc1ccc(-n2c([C@H]3CCCN3)nc3c(C4CC4)cc([N+](=O)[O-])cc3c2=O)cc1]

### Synthesis of tert-butyl (R)-2-(8-cyclopropyl-3-(4-methoxyphenyl)-6-nitro-4-oxo-3,4-dihydroquinazolin-2-yl) pyrrolidine-1-carboxylate (20b)

A mixture of 18a (20.00 mg, 0.036 mmol), cesium carbonate (23.49 mg, 0.072 mmol), cyclopropylboronic acid (4.75 mg, 0.055 mmol) and tetrakis(triphenylphosphine) palladium (0) (4.16 mg, 0.0036 mmol) in dioxane (1 mL) and H₂O (0.5 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure. The residue was purified by flash chromatography (0% Ethyl acetate/Hexane˜5% Ethyl acetate/Hexane) to afford 20b (10.40 mg, 57.1%) as pale yellow oil. ¹H NMR (400 MHz, CDCl₃) δ 8.90 (dd, J=5.9, 2.5 Hz, 1H), 7.98 (s, 1H), 7.49-7.16 (m, 1H), 7.18-7.02 (m, 4H), 4.65-4.56 (m, 1H), 3.89 (d, J=9.0 Hz, 3H), 3.80-3.67 (m, 1H), 3.56-3.48 (m, 1H), 2.93-2.80 (m, 1H), 2.14-1.80 (m, 3H), 1.42 (s, 3H), 1.27 (d, J=11.6 Hz, 6H), 1.28-1.14 (m, 2H), 0.96-0.85 (m, 3H). MS (ESI) m/z: 507.1 (M+1)⁺.

### Synthesis of (R)-8-cyclopropyl-3-(4-methoxyphenyl)-6-nitro-2-(pyrrolidin-2-yl) quinazolin-4 (3H)-one (21o)

Under nitrogen, trifluoroacetic acid (0.02 mL, 0.32 mmol) was added to a solution of 20b (8.00 mg, 0.016 mmol) in dry CH₂Cl₂ (0.5 mL) at 0° C., then stirred at room temperature overnight, then the solvent was removed in vacuo and the residue was diluted with CH₂Cl₂ (10 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 21o (3.80 mg, 58.4%) as pale yellow amorphous solid. ¹H NMR (500 MHz, CDCl₃) δ 8.90 (d, J=2.6 Hz, 1H), 7.98 (d, J=2.6 Hz, 1H), 7.24-7.20 (m, 1H), 7.19-7.15 (m, 1H), 7.07 (d, J=9.1 Hz, 2H), 3.96 (t, J=7.0 Hz, 1H), 3.89 (s, 3H), 3.31-3.25 (m, 1H), 2.96-2.88 (m, 1H), 2.86-2.81 (m, 1H), 2.01 (brs, 1H), 1.84-1.73 (m, 4H), 1.28-1.21 (m, 2H), 0.95-0.90 (m, 2H). MS (ESI) m/z: 407.1 (M+1)⁺.

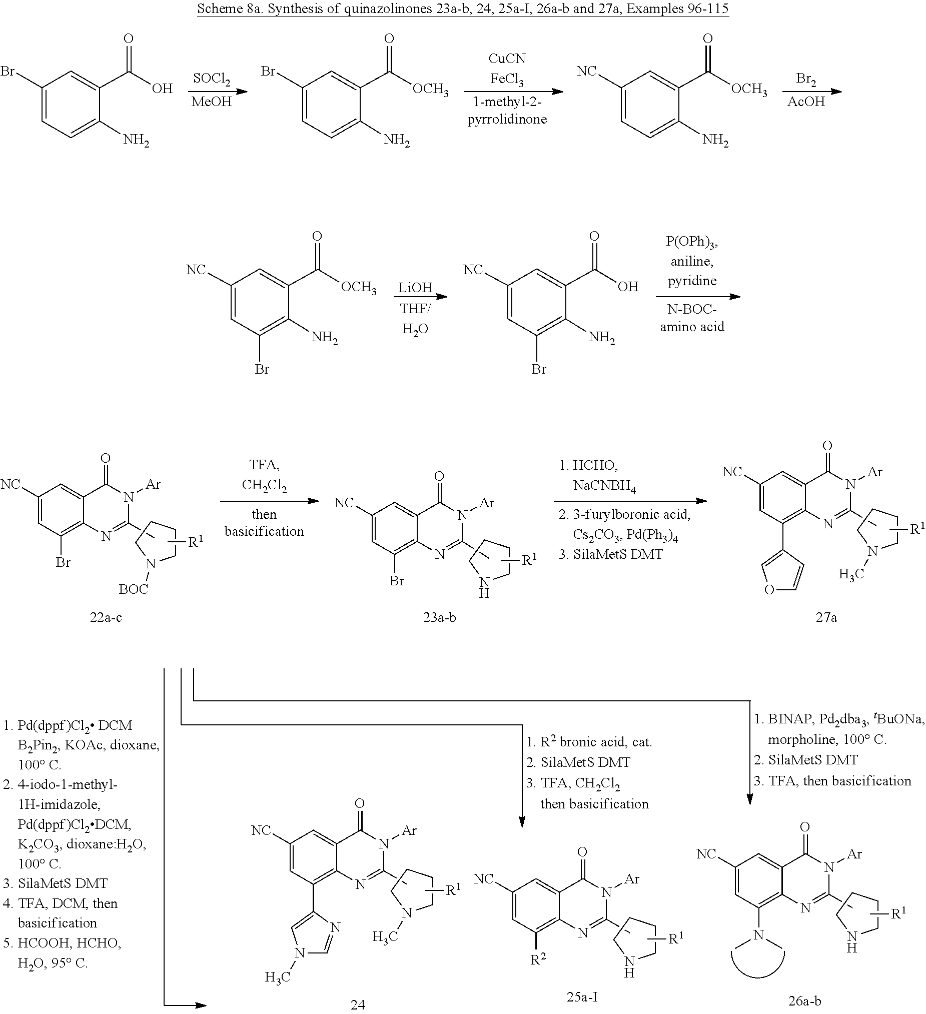

### Example 96. Synthesis of (R)-8-bromo-3-(3-fluoro-4-methoxyphenyl)-4-oxo-2-(pyrrolidin-2-yl)-3,4-dihydroquinazoline-6-carbonitrile (23a)

Prepared according to Scheme 8a.

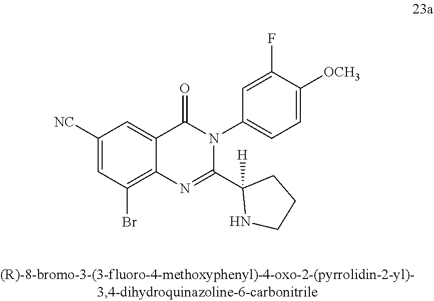 [SMILES: COc1ccc(-n2c([C@H]3CCCN3)nc3c(Br)cc(C#N)cc3c2=O)cc1F]

### Synthesis of methyl 2-amino-5-bromobenzoate

Under nitrogen, thionyl chloride (8.80 mL, 120.00 mmol) was added to a mixture of 2-amino-5-bromobenzoic acid (6.48 g, 30.00 mmol) in dry methanol (100 mL) at 0° C., then heated to reflux at room temperature for 36 h. After cooling down, the reaction was treated with water (100 mL) and extracted with ethyl acetate (3×100 mL). The combined organic layers were washed with saturated NaHCO₃ solution (100 mL), brine (100 mL), dried over anhydrous Na₂SO₄, and filtered, then the solvent was evaporated under reduced pressure. The residue was purified by flash chromatography (0% Ethyl acetate/Hexane to 20% Ethyl acetate/Hexane) to afford methyl 2-amino-5-bromobenzoate (5.76 g, 83.4%) as off-white solid. ¹H NMR (400 MHz, CDCl₃) δ 7.96 (d, J=2.3 Hz, 1H), 7.32 (dd, J=8.8, 2.4 Hz, 1H), 6.55 (d, J=8.8 Hz, 1H), 5.75 (s, 2H), 3.87 (s, 3H). MS (ESI) m/z: 231.1 (M+1)⁺.

### Synthesis of methyl 2-amino-5-cyanobenzoate

A mixture of methyl 2-amino-5-bromobenzoate (5.00 g, 21.70 mmol), copper cyanide (2.35 g, 26.00 mmol) in 1-methyl-2-pyrrolidinone (30 mL) was heated at 165° C. for 4 h. After cooling to room temperature, the reaction mixture was poured into a mixture of FeCl₃ (15.00 g, 92.48 mmol) and concentrated hydrochloric acid (4 mL) in water (30 mL), then stirred at 60° C. for 1 hour. After cooling down, the reaction mixture was extracted with ethyl acetate (3×100 mL). The combined organic layers were washed with water (100 mL), 1N sodium hydroxide solution (100 mL), brine (100 mL), dried over anhydrous Na₂SO₄, and filtered, then the solvent was evaporated under reduced pressure. The residue was purified by flash chromatography (0% ethyl acetate/hexane to 50% ethyl acetate/hexane) to afford methyl 2-amino-5-cyanobenzoate (2.65 g, 69.5%) as pale yellow solid. ¹H NMR (400 MHz, CDCl₃) δ 8.19 (d, J=1.6 Hz, 1H), 7.45 (dd, J=8.6, 1.7 Hz, 1H), 6.68 (d, J=8.7 Hz, 1H), 6.31 (s, 2H), 3.90 (s, 3H). MS (ESI) m/z: 177.1 (M+1)⁺.

### Synthesis of methyl 2-amino-3-bromo-5-cyanobenzoate

Under nitrogen, Bromine (0.75 mL, 14.29 mmol) was added to a reaction mixture of methyl 2-amino-5-cyanobenzoate (2.45 g, 13.91 mmol) in glacial acetic acid (30 mL) at room temperature, then stirred at room temperature for 4 h, then the mixture was poured into ice water and the resulting precipitate was collected by filtration. After washing the residue with water, the resulting solid was dissolved with acetone, then dried over with anhydrous Na₂SO₄, and filtered, then the solvent was evaporated under reduced pressure to afford methyl 2-amino-3-bromo-5-cyanobenzoate (3.16 g, 89.1%) as pale yellow solid. ¹H NMR (400 MHz, acetone-d₆) δ 8.18 (d, J=1.7 Hz, 1H), 8.00 (d, J=1.7 Hz, 1H), 3.92 (s, 3H). MS (ESI) m/z: 256.1 (M+1)⁺.

### Synthesis of 2-amino-3-bromo-5-cyanobenzoic Acid

At 0° C., lithium hydroxide (0.60 g, 24.80 mmol) was added into the solution of methyl 2-amino-3-bromo-5-cyanobenzoate (3.16 g, 12.40 mmol) in THF (60 mL) and H₂O (60 mL), then stirred at room temperature overnight. The mixture was then adjusted to pH 2 with 1.0N HCl and extracted with ethyl acetate (3×100 mL). The combined organic layers were washed with brine (100 mL) and dried over anhydrous Na₂SO₄, then concentrated under reduced pressure to afford 2-amino-3-bromo-5-cyanobenzoic acid (2.46 g, 82.3%) as pale yellow solid. ¹H NMR (400 MHz, Acetone-d₆) δ 8.23 (d, J=1.8 Hz, 1H), 8.01 (d, J=1.9 Hz, 1H), 7.43 (s, 2H). MS (ESI) m/z: 242.1 (M+1)⁺.

### Synthesis of tert-butyl (R)-2-(8-bromo-6-cyano-3-(3-fluoro-4-methoxyphenyl)-4-oxo-3,4-dihydroquinazolin-2-yl) pyrrolidine-1-carboxylate (22a)

2-amino-3-bromo-5-cyanobenzoic acid (0.16 g, 0.66 mmol) and Boc-D-proline (185.12 mg, 0.86 mmol) were dissolved in dry pyridine (4 mL). Triphenyl phosphite (0.52 mL, 1.98 mmol) was added to the mixture and the reaction mixture was heated at 110° C. with MWI for 40 min. 3-Fluoro-4-methoxyaniline (112.92 mg, 0.80 mmol) was added to the mixture and the reaction mixture was heated at 110° C. with MWI for 40 min. Pyridine was removed in vacuo and the residue was diluted with CH₂Cl₂ (100 mL) and washed with 1N HCl aqueous (50 mL). The organic layer was washed with H₂O (50 mL) and brine (50 mL), dried over anhydrous Na₂SO₄, and filtered, then the solvent was evaporated under reduced pressure. The residue was purified by flash chromatography (0% ethyl acetate/Hexane to 40% ethyl acetate/hexane) to afford 22a (0.10 g, 28.6%) as pale yellow solid. ¹H NMR (400 MHz, CDCl₃) δ 8.53-8.48 (m, 1H), 8.23-8.16 (m, 1H), 7.41-7.16 (m, 1H), 7.15-7.05 (m, 1H), 6.98-6.91 (m, 1H), 4.62-4.49 (m, 1H), 4.01-3.95 (m, 3H), 3.90-3.79 (m, 1H), 3.60-3.44 (m, 1H), 2.28-1.84 (m, 4H), 1.42 (d, J=3.9 Hz, 6H), 1.29 (d, J=2.1 Hz, 3H). MS (ESI) m/z: 544.1 (M+1)⁺.

### Synthesis of (R)-8-bromo-3-(3-fluoro-4-methoxyphenyl)-4-oxo-2-(pyrrolidin-2-yl)-3,4-dihydroquinazoline-6-carbonitrile (23a)

Under nitrogen, trifluoroacetic acid (0.12 mL, 1.50 mmol) was added to a solution of 22a (40.00 mg, 0.075 mmol) in dry CH₂Cl₂ (4 mL) at 0° C., then stirred at room temperature overnight, then the solvent was removed in vacuo and the residue was diluted with CH₂Cl₂ (30 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂ to 5% MeOH/CH₂Cl₂) to afford 23a (28.60 mg, 86.0%) as pale yellow solid. ¹H NMR (500 MHz, CDCl₃) δ 8.42 (s, 1H), 8.16 (s, 1H), 7.10-6.86 (m, 3H), 3.97-3.81 (m, 4H), 3.31-3.18 (m, 1H), 2.98 (brs, 1H), 2.81-2.78 (m, 1H), 1.86-1.68 (m, 4H). MS (ESI) m/z: 443.1 (M+1)⁺.

### Example 97. Synthesis of (S)-8-bromo-3-(3-fluoro-4-methoxyphenyl)-4-oxo-2-(pyrrolidin-2-yl)-3,4-dihydroquinazoline-6-carbonitrile (23b)

Prepared according to Scheme 8a.

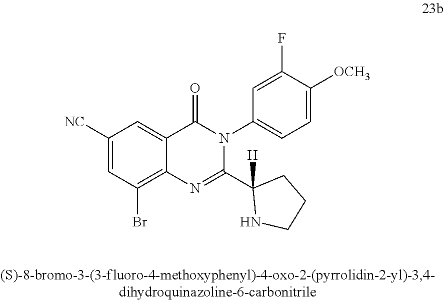 [SMILES: COc1ccc(-n2c([C@@H]3CCCN3)nc3c(Br)cc(C#N)cc3c2=O)cc1F]

### Synthesis of tert-butyl (S)-2-(8-bromo-6-cyano-3-(3-fluoro-4-methoxyphenyl)-4-oxo-3,4-dihydroquin-azolin-2-yl) pyrrolidine-1-carboxylate (22b)

2-amino-3-bromo-5-cyanobenzoic acid (0.16 g, 0.66 mmol) and Boc-L-proline (185.12 mg, 0.86 mmol) were dissolved in dry pyridine (4 mL). Triphenyl phosphite (0.52 mL, 1.98 mmol) was added to the mixture and the reaction mixture was heated at 110° C. with MWI for 40 min. 3-Fluoro-4-methoxyaniline (112.92 mg, 0.80 mmol) was added to the mixture and the reaction mixture was heated at 110° C. with MWI for 40 min. Pyridine was removed in vacuo and the residue was diluted with CH₂Cl₂ (100 mL) and washed with 1N HCl aqueous (50 mL). The organic layer was washed with H₂O (50 mL) and brine (50 mL), dried over anhydrous Na₂SO₄, and filtered, then the solvent was evaporated under reduced pressure. The residue was purified by flash chromatography (0% ethyl acetate/hexane to 40% ethyl acetate/hexane) to afford 22b (0.12 g, 33.5%) as pale yellow solid. ¹H NMR (400 MHz, CDCl₃) δ 8.56-8.48 (m, 1H), 8.24-8.16 (m, 1H), 7.41-7.16 (m, 1H), 7.15-7.06 (m, 1H), 6.96-6.88 (m, 1H), 4.62-4.48 (m, 1H), 4.01-3.95 (m, 3H), 3.91-3.79 (m, 1H), 3.65-3.45 (m, 1H), 2.28-1.85 (m, 4H), 1.42 (d, J=3.9 Hz, 6H), 1.28 (d, J=2.1 Hz, 3H). MS (ESI) m/z: 544.1 (M+1)⁺.

### Synthesis of (S)-8-bromo-3-(3-fluoro-4-methoxyphenyl)-4-oxo-2-(pyrrolidin-2-yl)-3,4-dihydroquinazoline-6-carbonitrile (23b)

Under nitrogen, trifluoroacetic acid (0.12 mL, 1.50 mmol) was added to a solution of 22b (40.00 mg, 0.075 mmol) in dry CH₂Cl₂ (4 mL) at 0° C., then stirred at room temperature overnight, then the solvent was removed in vacuo and the residue was diluted with CH₂Cl₂ (30 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 23b (20.50 mg, 61.6%) as a pale yellow amorphous solid. ¹H NMR (500 MHz, CDCl₃) δ 8.41 (s, 1H), 8.16 (s, 1H), 7.11-6.86 (m, 3H), 3.98-3.81 (m, 4H), 3.35-3.18 (m, 1H), 2.96 (brs, 1H), 2.81-2.78 (m, 1H), 1.91-1.68 (m, 4H). MS (ESI) m/z: 443.1 (M+1)⁺.

### Example 98. Synthesis of (R)-3-(3-fluoro-4-methoxyphenyl)-8-(1-methyl-1H-imidazol-4-yl)-2-(1-methylpyrrolidin-2-yl)-4-oxo-3,4-dihydroquinazoline-6-carbonitrile (24)

Prepared according to Scheme 8a.

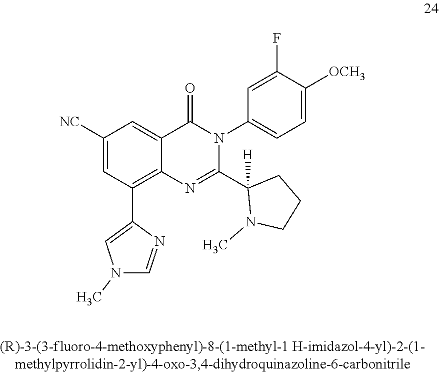 [SMILES: COc1ccc(-n2c([C@H]3CCCN3C)nc3c(-c4cn(C)cn4)cc(C#N)cc3c2=O)cc1F]

### Synthesis of tert-butyl (R)-2-(6-cyano-3-(3-fluoro-4-methoxyphenyl)-4-oxo-8-(4,4,5,5-tetramethyl-1,3,2-dioxaborolan-2-yl)-3,4-dihydroquinazolin-2-yl)pyrrolidine-1-carboxylate

A solution of tert-butyl (R)-2-(8-bromo-6-cyano-3-(3-fluoro-4-methoxyphenyl)-4-oxo-3,4-dihydroquinazolin-2-yl) pyrrolidine-1-carboxylate 22a (100 mg, 0.18 mmol), B₂Pin₂ (70 mg, 0.27 mmol) and KOAc (54 mg, 0.55 mmol) in dioxane (5 mL) was degassed with argon for 10 min. Pd(dppf)Cl₂-DCM (CAS: 95464-05-4) (15 mg, 0.018 mmol) was added, and the resulting reaction mixture was heated at 100° C. for 20 h. The reaction mixture was cooled to rt, diluted with EtOAc (4 mL) and brine solution (4 mL) was added. After separating the organic phase, the aqueous layer was further extracted with CH₂Cl₂ (2×3 mL). Combined organic layers were dried over Na₂SO₄ and concentrated under reduced pressure. The crude material was purified using column chromatography where the desired boronate ester intermediate (tert-butyl (R)-2-(6-cyano-3-(3-fluoro-4-methoxyphenyl)-4-oxo-8-(4,4,5,5-tetramethyl-1,3,2-dioxaborolan-2-yl)-3,4-dihydroquinazolin-2-yl)pyrrolidine-1-carboxylate) was obtained as brown colored oil (93 mg, 85%) which was carried forward to the next step.

### Synthesis of tert-butyl (R)-2-(6-cyano-3-(3-fluoro-4-methoxyphenyl)-8-(1-methyl-11H-imidazol-4-yl)-4-oxo-3,4-dihydroquinazolin-2-yl)pyrrolidine-1-carboxylate

A solution of the crude boronate ester (90 mg, 0.15 mmol), 4-iodo-1-methyl-1H-imidazole (CAS: 71759-87-0) (64 mg, 0.30 mmol) and K₂CO₃ (84 mg, 0.60 mmol) in 5:1 dioxane/H₂O (4.8 mL) was degassed with argon for 10 minutes. PdCl₂dppf (CAS: 72287-26-4) (12 mg, 0.015 mmol) was added in one portion and reaction heated at 100° C. for 16 h. The reaction was diluted with EtOAc (4 mL) and H₂O (1 mL), the phases were separated, and the aqueous layer was extracted with EtOAc (2×3 mL). Combined organic layers were dried over anhydrous Na₂SO₄, filtered, and the filtrate was concentrated in vacuo and purified by column chromatography (MPLC). After concentration of isolated product (confirmed by ¹H NMR), the product (30 mg) was dissolved in anhydrous CH₂Cl₂ (3 mL) and added with palladium scavenger—SilaMetS-DMT (100 mg) and heated to 45° C. for 5 h. The reaction mixture was cooled down, contents were filtered through celite (1 g) using sintered funnel and washed with CH₂Cl₂ (2×3 mL). The obtained filtrate was concentrated in vacuo to obtain solid compound. This palladium scavenging process was repeated for three times following the same procedure to obtain tert-butyl (R)-2-(6-cyano-3-(3-fluoro-4-methoxyphenyl)-8-(1-methyl-1H-imidazol-4-yl)-4-oxo-3,4-dihydroquinazolin-2-yl)pyrrolidine-1-carboxylate as white solid (28 mg, 37%). ¹H NMR (400 MHz, CDCl₃) δ 8.89-8.77 (m, 1H), 8.45-8.36 (m, 1H), 8.24-8.15 (m, 1H), 7.58-7.46 (m, 1H), 7.20-7.10 (m, 1H), 7.10-6.96 (m, 2H), 4.73-4.57 (m, 1H), 3.98 (dd, J=8.5, 1.2 Hz, 3H), 3.82 (d, J=2.8 Hz, 3H), 3.79-3.69 (m, 1H), 3.70-3.56 (m, 1H), 2.14-1.78 (m, 7H), 1.42 (d, J=3.0 Hz, 3H), 1.26 (d, J=14.5 Hz, 21H). MS (ESI) m/z: 545 (M+1)⁺.

### Synthesis of (R)-3-(3-fluoro-4-methoxyphenyl)-8-(1-methyl-1H-imidazol-4-yl)-2-(1-methylpyrrolidin-2-yl)-4-oxo-3,4-dihydroquinazoline-6-carbonitrile (24)

To a stirred solution of tert-butyl (R)-2-(6-cyano-3-(3-fluoro-4-methoxyphenyl)-8-(1-methyl-1H-imidazol-4-yl)-4-oxo-3,4-dihydroquinazolin-2-yl)pyrrolidine-1-carboxylate (28 mg, 0.05 mmol) in CH₂Cl₂ (3 mL), trifluoroacetic acid (0.1 mL) was added at 0° C. and the reaction mixture was stirred for 20 h at rt. The resulting mixture was concentrated in vacuo, and the residue was dissolved in 5 mL of CH₂Cl₂ then basified with saturated aqueous Na₂CO₃ solution till the pH=10. The phases were separated, and the aqueous layer was further extracted with CH₂Cl₂ (3×5 mL). The combined organic layer was dried over anhydrous Na₂SO₄, filtered, and concentrated in vacuo to give a crude material. Without purification, water (1 mL), HCOOH (98%, 0.5 mL) and HCHO (37% aqueous solution, 0.1 mL) were added to the crude material obtained above. Then the resulting mixture was heated to 95° C. After 5 h, the reaction mixture was cooled and made alkaline using 1N NaOH solution and saturated aqueous Na₂CO₃ solution, followed by extraction with 10% MeOH/CH₂Cl₂ (2×5 mL). The combined organic layer was dried over anhydrous Na₂SO₄, filtered, concentrated in vacuo and purified by reverse phase column chromatography using a C18 column to afford (R)-3-(3-fluoro-4-methoxyphenyl)-8-(1-methyl-1H-imidazol-4-yl)-2-(1-methylpyrrolidin-2-yl)-4-oxo-3,4-dihydroquinazoline-6-carbonitrile 24 (6 mg, 26% yield). ¹H NMR (400 MHz, CDCl₃) δ 8.80 (d, J=2.1 Hz, 1H), 8.64 (dd, J=3.5, 1.3 Hz, 1H), 8.29 (d, J=2.1 Hz, 1H), 7.45 (d, J=1.3 Hz, 1H), 7.06 (td, J=8.6, 5.9 Hz, 1H), 6.94 (tdd, J=11.1, 5.2, 2.5 Hz, 2H), 3.92 (d, J=1.3 Hz, 3H), 3.71 (s, 3H), 3.11 (ddd, J=15.0, 11.5, 7.1 Hz, 2H), 2.30 (d, J=3.4 Hz, 3H), 2.28-2.17 (m, 1H), 1.94-1.76 (m, 3H), 1.68 (s, 1H). MS (ESI) m/z: 459.1 (M+1)⁺.

### Example 99. Synthesis of (R)-3-(3-fluoro-4-methoxyphenyl)-8-(6-fluoropyridin-3-yl)-4-oxo-2-(pyrrolidin-2-yl)-3,4-dihydroquinazoline-6-carbonitrile (25a)

Prepared according to Scheme 8a.

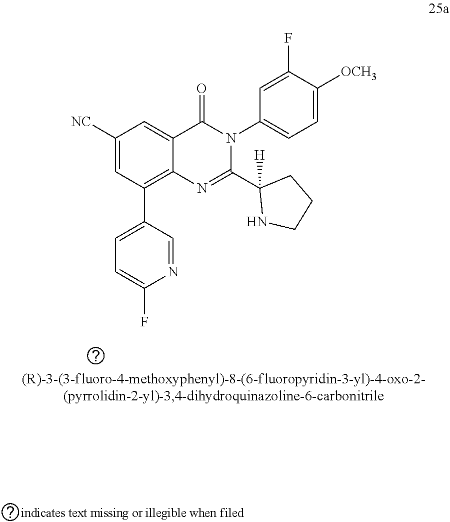 [SMILES: COc1ccc(-n2c([C@H]3CCCN3)nc3c(-c4ccc(F)nc4)cc(C#N)cc3c2=O)cc1F]

### Synthesis of (R)-3-(3-fluoro-4-methoxyphenyl)-8-(6-fluoropyridin-3-yl)-4-oxo-2-(pyrrolidin-2-yl)-3,4-dihydroquinazoline-6-carbonitrile (25a)

A mixture of 22a (40.75 mg, 0.075 mmol), cesium carbonate (48.86 mg, 0.15 mmol), 2-fluoropyridine-5-boronic acid (15.50 mg, 0.11 mmol) and tetrakis(tri-phenylphosphine) palladium (0) (8.66 mg, 0.0075 mmol) in dioxane (3 mL) and H₂O (1 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.05 mL, 0.80 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (40 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂ to 5% MeOH/CH₂Cl₂) to afford 25a (8.65 mg, 25.1%) as pale yellow solid. ¹H NMR (500 MHz, CDCl₃) δ 8.63 (d, J=1.9 Hz, 1H), 8.48 (d, J=2.1 Hz, 1H), 8.07 (td, J=8.1, 2.4 Hz, 1H), 7.99 (d, J=1.8 Hz, 1H), 7.16-7.12 (m, 1H), 7.11-7.09 (m, 1H), 7.08-7.04 (m, 1H), 7.03-6.98 (m, 1H), 3.98 (s, 4H), 3.90 (dd, J=13.9, 7.4 Hz, 1H), 3.10-3.04 (m, 1H), 2.81-2.75 (m, 1H), 2.05 (brs, 1H), 1.85-1.65 (m, 4H). MS (ESI) m/z: 460.1 (M+1)⁺.

### Example 100. Synthesis of (R)-3-(3-fluoro-4-methoxyphenyl)-4-oxo-2-(pyrrolidin-2-yl)-8-(6-(trifluoromethyl)pyridin-3-yl)-3,4-dihydroquinazoline-6-carbonitrile (25b)

Prepared according to Scheme 8a.

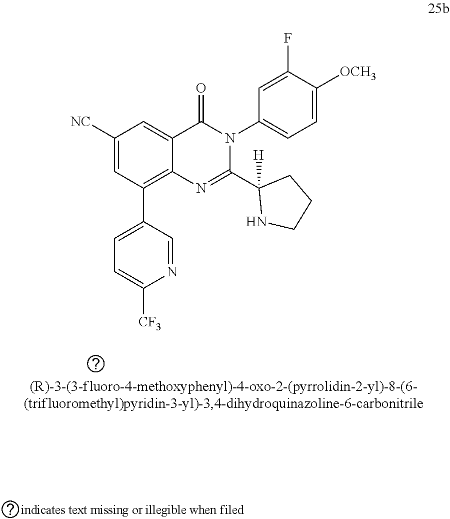 [SMILES: COc1ccc(-n2c([C@H]3CCCN3)nc3c(-c4ccc(C(F)(F)F)nc4)cc(C#N)cc3c2=O)cc1F]

### Synthesis of (R)-3-(3-fluoro-4-methoxyphenyl)-4-oxo-2-(pyrrolidin-2-yl)-8-(6-(trifluoromethyl)pyridin-3-yl)-3,4-dihydroquinazoline-6-carbonitrile (25b)

A mixture of 22a (40.75 mg, 0.075 mmol), cesium carbonate (48.86 mg, 0.15 mmol), 2-trifluoromethyl-5-pyridineboric acid (21.00 mg, 0.11 mmol) and tetrakis(triphenylphosphine) palladium (0) (8.66 mg, 0.0075 mmol) in dioxane (3 mL) and H₂O (1 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.05 mL, 0.80 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (40 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂ to 5% MeOH/CH₂Cl₂) to afford 25b (10.65 mg, 27.8%) as pale yellow solid. ¹H NMR (500 MHz, CDCl₃) δ 8.99 (d, J=1.5 Hz, 1H), 8.67 (d, J=1.9 Hz, 1H), 8.14 (dd, J=8.1, 1.9 Hz, 1H), 8.01 (dd, J=1.9, 0.9 Hz, 1H), 7.83 (d, J=10.6 Hz, 1H), 7.16-7.11 (m, 1H), 7.10-7.05 (m, 1H), 7.04-6.98 (m, 1H), 3.98 (s, 3H), 3.95-3.88 (m, 1H), 3.08-3.01 (m, 1H), 2.81-2.75 (m, 1H), 2.06 (brs, 1H), 1.85-1.68 (m, 4H). MS (ESI) m/z: 510.1 (M+1)⁺.

### Example 101. Synthesis of 3-(3-fluoro-4-methoxyphenyl)-8-(6-methylpyridin-3-yl)-4-oxo-2-(pyrrolidin-2-yl)-3,4-dihydroquinazoline-6-carbonitrile (25c)

Prepared according to Scheme 8a.

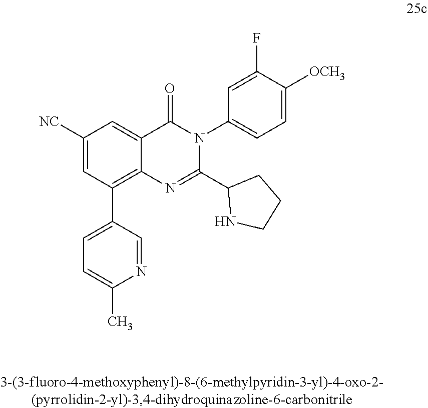 [SMILES: COc1ccc(-n2c(C3CCCN3)nc3c(-c4ccc(C)nc4)cc(C#N)cc3c2=O)cc1F]

### Synthesis of (3-fluoro-4-methoxyphenyl)-8-(6-methylpyridin-3-yl)-4-oxo-2-(pyrrolidin-2-yl)-3,4-dihydroquinazoline-6-carbonitrile (25c)

A mixture of 22a (40.75 mg, 0.075 mmol), cesium carbonate (48.86 mg, 0.15 mmol), 6-methylpyridine-3-boronic acid (15.06 mg, 0.11 mmol) and tetrakis(triphenylphosphine) palladium (0) (8.66 mg, 0.0075 mmol) in dioxane (3 mL) and H₂O (1 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger in triplicate at elevated temperature to remove any residual metal from the coupling reaction. The crude product was separated from the scavenger by filtration and used in the next step.

Under nitrogen, trifluoroacetic acid (0.05 mL, 0.80 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (40 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 25c (8.96 mg, 26.3%) as white solid. ¹H NMR (500 MHz, CDCl₃) δ 8.75 (d, J=2.0 Hz, 1H), 8.60 (d, J=1.9 Hz, 1H), 7.98 (d, J=1.2 Hz, 1H), 7.84 (dt, J=13.0, 6.5 Hz, 1H), 7.30 (d, J=8.0 Hz, 1H), 7.16-7.11 (m, 1H), 7.10-7.06 (m, 1H), 7.05-6.98 (m, 1H), 3.98 (s, 3H), 3.91-3.86 (m, 1H), 3.11-3.05 (m, 1H), 2.78-2.75 (m, 1H), 2.67 (s, 3H), 2.01 (brs, 1H), 1.81-1.68 (m, 4H). MS (ESI) m/z: 456.1 (M+1)⁺. Assessment of % ee showed a scalemic mixture. Isomers were separated by chiral HPLC and characterized as individual compounds.

### Example 102. Synthesis of (R)-3-(3-fluoro-4-methoxyphenyl)-8-(6-methylpyridin-3-yl)-4-oxo-2-(pyrrolidin-2-yl)-3,4-dihydroquinazoline-6-carbonitrile (25d)

Prepared according to Scheme 8a.

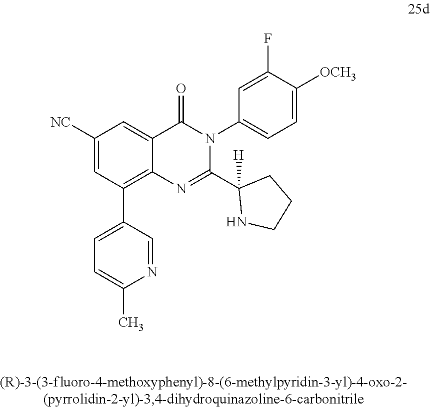 [SMILES: COc1ccc(-n2c([C@H]3CCCN3)nc3c(-c4ccc(C)nc4)cc(C#N)cc3c2=O)cc1F]

(R)-3-(3-fluoro-4-methoxyphenyl)-8-(6-methylpyridin-3-yl)-4-oxo-2-(pyrrolidin-2-yl)-3,4-dihydroquinazoline-6-carbonitrile (25d). Compound 25c, obtained as a scalemic mixture of enantiomers, was subjected to preparative chiral HPLC (Regispak 5 micron 25 cm×21.1 mm column, 90:10 hexanes:isopropanol, 10 mL/min flow rate, r.t.=20.5 for S-25e and 23 min for R-25d). 11 mg (0.024 mmol) of (S)-25e and 9 mg (0.020 mmol) of (R)-25d were obtained separately. e.e. of both enantiomers >99% (Regispak 5 micron 25 cm×4.6 mm column, 70:30 hexanes:isopropanol, 1 mL/min flow rate, r.t.=9 min for S-25e and 11.3 min for R-25d). ¹H NMR (400 MHz, CDCl₃) δ 8.74 (d, J=2.2 Hz, 1H), 8.65-8.58 (m, 1H), 7.98 (t, J=1.9 Hz, 1H), 7.83 (dt, J=8.0, 2.7 Hz, 1H), 7.31 (d, J=8.0 Hz, 1H), 7.18-7.06 (m, 2H), 7.05-6.98 (m, 1H), 4.07 (t, J=7.1 Hz, 1H), 3.98 (s, 3H), 3.11 (dt, J=9.2, 5.6 Hz, 1H), 2.84 (tt, J=13.2, 6.4 Hz, 1H), 2.67 (s, 3H), 2.37 (brs, 1H), 1.85-1.73 (m, 4H). MS (ESI) m/z: 456.2 (M+1)⁺.

### Example 103. Synthesis of (S)-3-(3-fluoro-4-methoxyphenyl)-8-(6-methylpyridin-3-yl)-4-oxo-2-(pyrrolidin-2-yl)-3,4-dihydroquinazoline-6-carbonitrile (25e)

Prepared according to Scheme 8a.

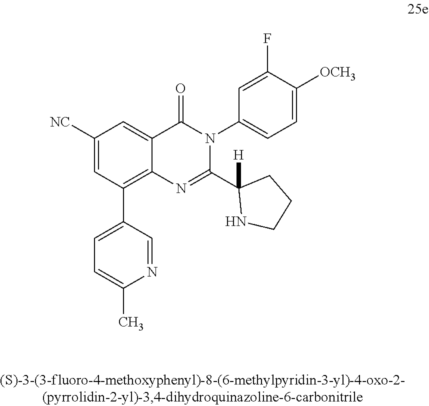 [SMILES: COc1ccc(-n2c([C@@H]3CCCN3)nc3c(-c4ccc(C)nc4)cc(C#N)cc3c2=O)cc1F]

(S)-3-(3-fluoro-4-methoxyphenyl)-8-(6-methylpyridin-3-yl)-4-oxo-2-(pyrrolidin-2-yl)-3,4-dihydroquinazoline-6-carbonitrile (25e). Compound 25c, obtained as a scalemic mixture of enantiomers, was subjected to preparative chiral HPLC (Regispak 5 micron 25 cm×21.1 mm column, 90:10 hexanes:isopropanol, 10 mL/min flow rate, r.t.=20.5 for S-25e and 23 min for R-25d). 11 mg (0.024 mmol) of (S)-25e and 9 mg (0.020 mmol) of (R)-25d obtained. e.e. of both enantiomers >99% (Regispak 5 micron 25 cm×4.6 mm column, 70:30 hexanes:isopropanol, 1 mL/min flow rate, r.t.=9 min for S-25e and 11.3 min for R-25d). ¹H NMR (400 MHz, CDCl₃) δ 8.74 (d, J=2.2 Hz, 1H), 8.65-8.58 (m, 1H), 7.98 (t, J=1.9 Hz, 1H), 7.83 (dt, J=8.0, 2.7 Hz, 1H), 7.31 (d, J=8.0 Hz, 1H), 7.18-7.06 (m, 2H), 7.05-6.98 (m, 1H), 4.07 (t, J=7.1 Hz, 1H), 3.98 (s, 3H), 3.11 (dt, J=9.2, 5.6 Hz, 1H), 2.84 (tt, J=13.2, 6.4 Hz, 1H), 2.67 (s, 3H), 2.37 (brs, 1H), 1.85-1.73 (m, 4H). MS (ESI) m/z: 456.2 (M+1)⁺.

### Example 104. Synthesis of (R)-3-(3-fluoro-4-methoxyphenyl)-8-(2-fluoropyridin-4-yl)-4-oxo-2-(pyrrolidin-2-yl)-3,4-dihydroquinazoline-6-carbonitrile (25f)

Prepared according to Scheme 8a.

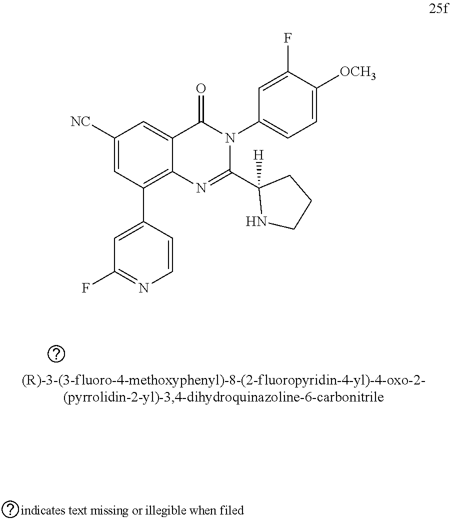 [SMILES: COc1ccc(-n2c([C@H]3CCCN3)nc3c(-c4ccnc(F)c4)cc(C#N)cc3c2=O)cc1F]

### Synthesis of (R)-3-(3-fluoro-4-methoxyphenyl)-8-(2-fluoropyridin-4-yl)-4-oxo-2-(pyrrolidin-2-yl)-3,4-dihydroquinazoline-6-carbonitrile (25f)

A mixture of 22a (40.75 mg, 0.075 mmol), Cesium carbonate (48.86 mg, 0.15 mmol), 2-fluoropyridine-4-boronic acid (15.50 mg, 0.11 mmol) and tetrakis(tri-phenylphosphine) palladium (0) (8.66 mg, 0.0075 mmol) in dioxane (3 mL) and H₂O (1 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.05 mL, 0.80 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (40 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 25f (10.56 mg, 30.6%) as pale yellow solid. ¹H NMR (500 MHz, CDCl₃) δ 8.66 (d, J=1.8 Hz, 1H), 8.36 (d, J=5.2 Hz, 1H), 8.02 (d, J=1.7 Hz, 1H), 7.44 (d, J=5.1 Hz, 1H), 7.24 (s, 1H), 7.16-7.11 (m, 1H), 7.10-7.06 (m, 1H), 7.05-6.98 (m, 1H), 3.98 (s, 3H), 3.91 (dd, J=13.8, 7.5 Hz, 1H), 3.13-3.01 (m, 1H), 2.81-2.75 (m, 1H), 2.18 (brs, 1H), 1.85-1.70 (m, 4H). MS (ESI) m/z: 460.1 (M+1)⁺.

### Example 105. Synthesis of (R)-3-(3-fluoro-4-methoxyphenyl)-4-oxo-8-(pyrimidin-5-yl)-2-(pyrrolidin-2-yl)-3,4-dihydroquinazoline-6-carbonitrile (25g)

Prepared according to Scheme 8a.

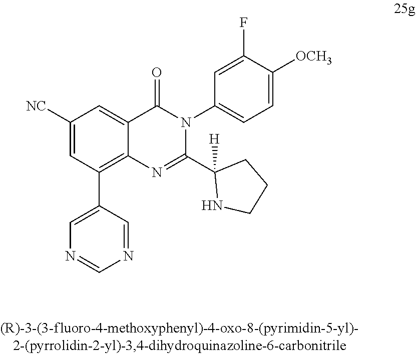 [SMILES: COc1ccc(-n2c([C@H]3CCCN3)nc3c(-c4cncnc4)cc(C#N)cc3c2=O)cc1F]

### Synthesis of (R)-3-(3-fluoro-4-methoxyphenyl)-4-oxo-8-(pyrimidin-5-yl)-2-(pyrrolidin-2-yl)-3,4-dihydroquinazoline-6-carbonitrile (25g)

A mixture of 22a (40.75 mg, 0.075 mmol), Cesium carbonate (48.86 mg, 0.15 mmol), 5-pyrimidinylboronic acid (13.65 mg, 0.11 mmol) and tetrakis(tri-phenylphosphine) palladium (0) (8.66 mg, 0.0075 mmol) in dioxane (2.5 mL) and H₂O (1 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.06 mL, 0.92 mmol) was added to a solution of pale yellow oil described above in dry CH₂Cl₂ (3 mL) at 0° C., then stirred at room temperature overnight, then the solvent was removed in vacuo and the residue was diluted with CH₂Cl₂ (40 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 25g (10.65 mg, 32.1%) as pale yellow amorphous solid. ¹H NMR (500 MHz, CDCl₃) δ 9.30 (s, 1H), 9.03 (s, 2H), 8.67 (d, J=1.9 Hz, 1H), 8.02 (d, J=1.4 Hz, 1H), 7.18-6.96 (m, 3H), 3.98 (s, 3H), 3.91 (dd, J=13.4, 6.5 Hz, 1H), 3.11-3.03 (m, 1H), 2.81-2.75 (m, 1H), 2.08 (brs, 1H), 1.86-1.68 (m, 4H). MS (ESI) m/z: 443.1 (M+1)⁺.

### Example 106. Synthesis of (R)-3-(3-fluoro-4-methoxyphenyl)-8-(5-fluoropyridin-3-yl)-4-oxo-2-(pyrrolidin-2-yl)-3,4-dihydroquinazoline-6-carbonitrile (25 h)

Prepared according to Scheme 8a.

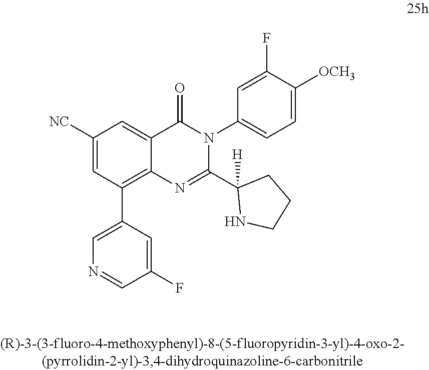 [SMILES: COc1ccc(-n2c([C@H]3CCCN3)nc3c(-c4cncc(F)c4)cc(C#N)cc3c2=O)cc1F]

### Synthesis of (R)-3-(3-fluoro-4-methoxyphenyl)-8-(5-fluoropyridin-3-yl)-4-oxo-2-(pyrrolidin-2-yl)-3,4-dihydroquinazoline-6-carbonitrile (25h)

A mixture of 22a (40.75 mg, 0.075 mmol), cesium carbonate (48.86 mg, 0.15 mmol), 5-fluoropyridin-3-ylboronic acid (15.50 mg, 0.11 mmol) and tetrakis(triphenylphosphine) palladium (0) (8.66 mg, 0.0075 mmol) in dioxane (3 mL) and H₂O (1 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.05 mL, 0.80 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (40 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 25 h (9.65 mg, 28.1%) as pale yellow solid. ¹H NMR (500 MHz, CDCl₃) δ 8.68 (s, 1H), 8.64 (d, J=1.9 Hz, 1H), 8.58 (d, J=2.6 Hz, 1H), 8.05-8.01 (m, 1H), 7.78-7.71 (m, 1H), 7.16-7.11 (m, 1H), 7.10-7.05 (m, 1H), 7.05-6.98 (m, 1H), 3.98 (s, 3H), 3.98-3.92 (m, 1H), 3.12-3.06 (m, 1H), 2.84-2.76 (m, 1H), 2.18 (brs, 1H), 1.86-1.68 (m, 4H). MS (ESI) m/z: 460.1 (M+1)⁺.

### Example 107. Synthesis of (R)-3-(3-fluoro-4-methoxyphenyl)-4-oxo-8-(pyridin-3-yl)-2-(pyrrolidin-2-yl)-3,4-dihydroquinazoline-6-carbonitrile (25i)

Prepared according to Scheme 8a.

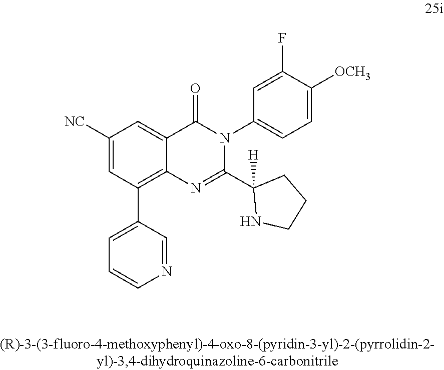 [SMILES: COc1ccc(-n2c([C@H]3CCCN3)nc3c(-c4cccnc4)cc(C#N)cc3c2=O)cc1F]

### Synthesis of (R)-3-(3-fluoro-4-methoxyphenyl)-4-oxo-8-(pyridin-3-yl)-2-(pyrrolidin-2-yl)-3,4-dihydroquinazoline-6-carbonitrile (25i)

A mixture of 22a (30.00 mg, 0.055 mmol), cesium carbonate (35.86 mg, 0.11 mmol), pyridine-3-boronic acid (10.36 mg, 0.084 mmol) and tetrakis(triphenylphosphine) palladium (0) (6.96 mg, 0.006 mmol) in dioxane (1.6 mL) and H₂O (0.8 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger ×3 at elevated temperature to remove any residual metal from the coupling reaction. The crude product was separated from the scavenger by filtration and used in the next step.

Under nitrogen, trifluoroacetic acid (0.04 mL, 0.50 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (30 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 25i (6.16 mg, 25.4%) as pale yellow solid. ¹H NMR (500 MHz, CDCl₃) δ 8.87 (s, 1H), 8.70 (d, J=4.4 Hz, 1H), 8.63 (s, 1H), 8.00 (d, J=0.8 Hz, 1H), 7.94 (d, J=7.8 Hz, 1H), 7.45 (dd, J=7.7, 4.9 Hz, 1H), 7.15-7.01 (m, 3H), 3.98 (s, 3H), 3.95-3.85 (m, 1H), 3.08-3.01 (m, 1H), 2.81-2.68 (m, 1H), 1.86-1.61 (m, 4H). MS (ESI) m/z: 442.1 (M+1)⁺.

### Example 108. Synthesis of (S)-3-(3-fluoro-4-methoxyphenyl)-4-oxo-8-(pyridin-3-yl)-2-(pyrrolidin-2-yl)-3,4-dihydroquinazoline-6-carbonitrile (25j)

Prepared according to Scheme 8a.

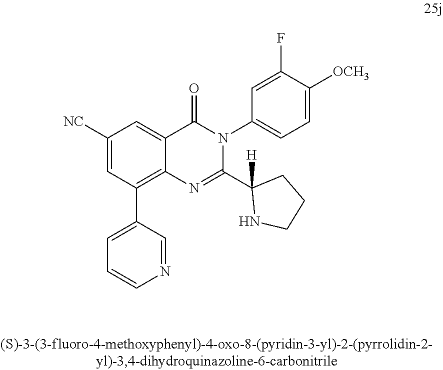 [SMILES: COc1ccc(-n2c([C@@H]3CCCN3)nc3c(-c4cccnc4)cc(C#N)cc3c2=O)cc1F]

### Synthesis of (S)-3-(3-fluoro-4-methoxyphenyl)-4-oxo-8-(pyridin-3-yl)-2-(pyrrolidin-2-yl)-3,4-dihydroquinazoline-6-carbonitrile (25j)

A mixture of 22b (30.00 mg, 0.055 mmol), cesium carbonate (35.86 mg, 0.11 mmol), pyridine-3-boronic acid (10.36 mg, 0.084 mmol) and tetrakis(triphenylphosphine) palladium (0) (6.96 mg, 0.006 mmol) in dioxane (1.6 mL) and H₂O (0.8 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.04 mL, 0.50 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (30 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl_{2˜}5% MeOH/CH₂Cl₂) to afford 25j (4.68 mg, 19.5%) as pale yellow solid. ¹H NMR (500 MHz, CDCl₃) δ 8.88 (s, 1H), 8.71 (d, J=4.5 Hz, 1H), 8.65 (s, 1H), 8.01 (d, J=0.8 Hz, 1H), 7.95 (d, J=7.8 Hz, 1H), 7.45 (dd, J=7.7, 4.9 Hz, 1H), 7.16-7.01 (m, 3H), 3.98 (s, 3H), 3.96-3.85 (m, 1H), 3.11-3.01 (m, 1H), 2.28-2.68 (m, 1H), 1.86-1.61 (m, 4H). MS (ESI) m/z: 442.1 (M+1)⁺.

### Example 109. Synthesis of (R)-3-(3-fluoro-4-methoxyphenyl)-4-oxo-8-(pyridin-4-yl)-2-(pyrrolidin-2-yl)-3,4-dihydroquinazoline-6-carbonitrile (25k)

Prepared according to Scheme 8a.

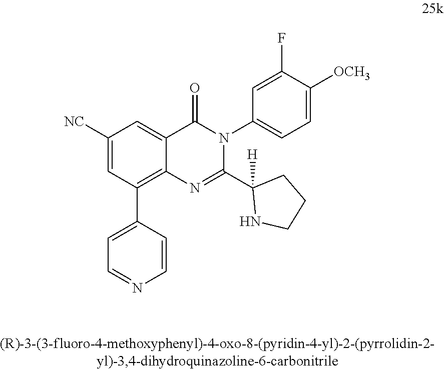 [SMILES: COc1ccc(-n2c([C@H]3CCCN3)nc3c(-c4ccncc4)cc(C#N)cc3c2=O)cc1F]

### Synthesis of (R)-3-(3-fluoro-4-methoxyphenyl)-4-oxo-8-(pyridin-4-yl)-2-(pyrrolidin-2-yl)-3,4-dihydroquinazoline-6-carbonitrile (25k)

A mixture of 22a (30.00 mg, 0.055 mmol), cesium carbonate (35.86 mg, 0.11 mmol), pyridine-4-boronic acid (10.36 mg, 0.084 mmol) and tetrakis(triphenylphosphine) palladium (0) (6.96 mg, 0.006 mmol) in dioxane (1.6 mL) and H₂O (0.8 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger at elevated temperature to remove any residual metal from the coupling reaction. The crude product was separated from the scavenger by filtration and used in the next step.

Under nitrogen, trifluoroacetic acid (0.04 mL, 0.50 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (30 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂˜5% MeOH/CH₂Cl₂) to afford 25k (6.86 mg, 28.2%) as pale yellow solid. ¹H NMR (400 MHz, CDCl₃) δ 8.76 (d, J=5.3 Hz, 2H), 8.65 (s, 1H), 8.01 (s, 1H), 7.54 (d, J=5.4 Hz, 2H), 7.18-6.96 (m, 3H), 4.05-3.83 (m, 4H), 3.08-3.01 (m, 1H), 2.81-2.68 (m, 1H), 2.52 (brs, 1H), 1.85-1.61 (m, 4H). MS (ESI) m/z: 442.1 (M+1)⁺.

### Example 110. Synthesis of (S)-3-(3-fluoro-4-methoxyphenyl)-4-oxo-8-(pyridin-4-yl)-2-(pyrrolidin-2-yl)-3,4-dihydroquinazoline-6-carbonitrile (25l)

Prepared according to Scheme 8a.

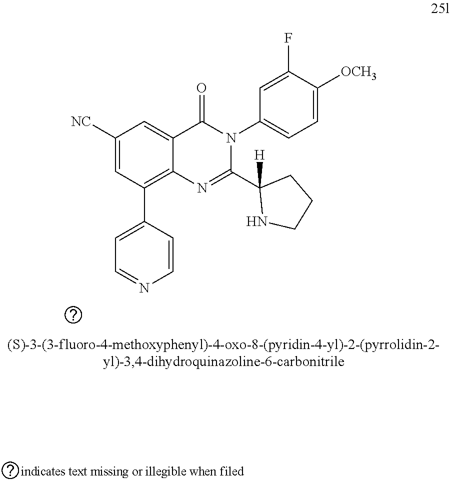 [SMILES: COc1ccc(-n2c([C@@H]3CCCN3)nc3c(-c4ccncc4)cc(C#N)cc3c2=O)cc1F]

### Synthesis of (S)-3-(3-fluoro-4-methoxyphenyl)-4-oxo-8-(pyridin-4-yl)-2-(pyrrolidin-2-yl)-3,4-dihydroquinazoline-6-carbonitrile (25l)

A mixture of 22b (30.00 mg, 0.055 mmol), cesium carbonate (35.86 mg, 0.11 mmol), Pyridine-4-boronic acid (10.36 mg, 0.084 mmol) and tetrakis(triphenylphosphine) palladium (0) (6.96 mg, 0.006 mmol) in dioxane (1.6 mL) and H₂O (0.8 mL) was degassed and flushed with nitrogen. The mixture was heated at 85° C. for 6 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger ×3 at elevated temperature to remove any residual metal from the coupling reaction. The crude product was separated from the scavenger by filtration and used in the next step.

Under nitrogen, trifluoroacetic acid (0.04 mL, 0.50 mmol) was added to a solution of pale yellow oil described above in dry CH₂Cl₂ (2 mL) at 0° C., then stirred at room temperature overnight, then the solvent was removed in vacuo and the residue was diluted with CH₂Cl₂ (30 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂-5% MeOH/CH₂Cl₂) to afford 251 (6.16 mg, 25.4%) as pale yellow solid. ¹H NMR (400 MHz, CDCl₃) δ 8.75 (d, J=5.4 Hz, 2H), 8.65 (s, 1H), 8.01 (s, 1H), 7.56 (d, J=5.4 Hz, 2H), 7.21-6.96 (m, 3H), 4.06-3.83 (m, 4H), 3.08-3.01 (m, 1H), 2.81-2.65 (m, 1H), 2.51 (brs, 1H), 1.86-1.61 (m, 4H). MS (ESI) m/z: 442.1 (M+1)⁺.

### Example 111. Synthesis of 3-(3-fluoro-4-methoxyphenyl)-8-(6-methylpyridin-3-yl)-2-(1-methylpyrrolidin-2-yl)-4-oxo-3,4-dihydroquinazoline-6-carbonitrile (25m)

Prepared as shown in Scheme 8a.

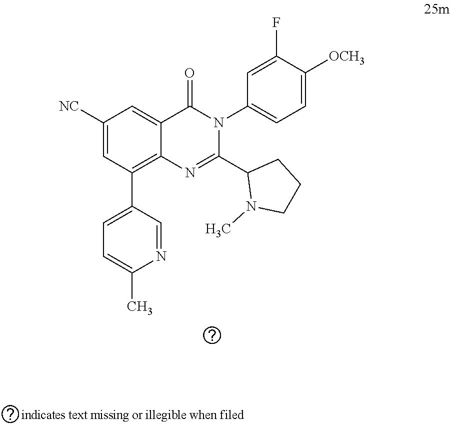 [SMILES: COc1ccc(-n2c(C3CCCN3C)nc3c(-c4ccc(C)nc4)cc(C#N)cc3c2=O)cc1F]

A solution of 25c in H₂O (1 mL) was subjected to sequential addition of HCHO (37% aqueous) (0.1 mL) and HCOOH (0.5 mL). The resulting reaction mixture was heated to 95° C. for 6 h. The reaction was cooled to rt and basified with 1N NaOH until pH˜10. A white precipitate crashed out which was extracted with CH₂Cl₂ (3×3 mL). The combined organic layer was dried over anhydrous Na₂SO₄, filtered, concentrated in vacuo to give a crude material which was subjected to column chromatography to afford 3-(3-fluoro-4-methoxyphenyl)-8-(6-methylpyridin-3-yl)-2-(1-methylpyrrolidin-2-yl)-4-oxo-3,4-dihydroquinazoline-6-carbonitrile 25m. as white solid (18 mg, 59%). ¹H NMR (400 MHz, CDCl₃) δ 8.77 (d, J=2.3 Hz, 11H), 8.60 (d, J=2.0 Hz, 11H), 7.97 (d, J=1.9 Hz, 1H), 7.89 (dd, J=8.0, 2.4 Hz, 1H), 7.28 (d, J=8.1 Hz, 1H), 7.19-7.06 (m, 2H), 7.01-6.89 (m, 1H), 3.98 (d, J=1.3 Hz, 3H), 3.59-3.46 (m, 1H), 2.98-2.86 (m, 1H), 2.65 (s, 3H), 2.44 (dtd, J=9.2, 7.2, 5.2 Hz, 1H), 2.20 (d, J=1.6 Hz, 3H), 2.09-1.89 (m, 2H), 1.88-1.71 (m, 2H). MS (ESI) m/z: 470.1 (M+1)⁺.

### Example 112. Synthesis of 3-(3-fluoro-4-methoxyphenyl)-2-((2R,4S)-4-fluoropyrrolidin-2-yl)-8-(6-methylpyridin-3-yl)-4-oxo-3,4-dihydroquinazoline-6-carbonitrile (25n)

Prepared as shown in Scheme 8a

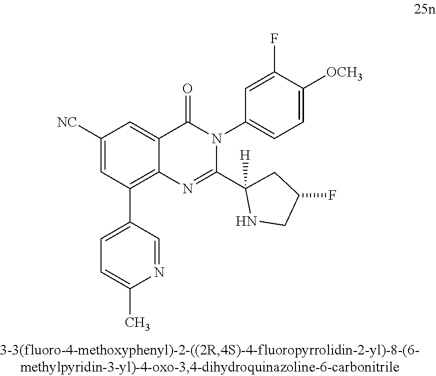 [SMILES: COc1ccc(-n2c([C@H]3C[C@H](F)CN3)nc3c(-c4ccc(C)nc4)cc(C#N)cc3c2=O)cc1F]

Step 1. Synthesis of tert-butyl (2R,4S)-2-(8-bromo-6-cyano-3-(3-fluoro-4-methoxyphenyl)-4-oxo-3,4-dihydroquinazolin-2-yl)-4-fluoropyrrolidine-1-carboxylate (22c). 2-amino-3-bromo-5-cyanobenzoic acid (0.35 g, 1.46 mmol) and (2R,4S)-1-(tert-butoxycarbonyl)-4-fluoropyrrolidine-2-carboxylic acid (442 mg, 1.90 mmol) were dissolved in dry pyridine (5 mL). Triphenyl phosphite (1.15 mL, 4.38 mmol) was added to the mixture and the reaction mixture was heated at 110° C. with MWI for 90 min. 3-Fluoro-4-methoxyaniline (247 mg, 1.75 mmol) was added to the mixture and the reaction mixture was heated at 100° C. with MWI for 30 min. Pyridine was removed in vacuo and the residue was diluted with CH₂Cl₂ (100 mL) and washed with 1N HCl aqueous (50 mL). The organic layer was washed with H₂O (50 mL) and brine (50 mL), dried over anhydrous Na₂SO₄, and filtered, then the solvent was evaporated under reduced pressure. The residue was purified by flash chromatography (0% solvent A (ethyl acetate:CH₂Cl₂=5:1)/hexane to 20% solvent A (ethyl acetate: CH₂Cl₂=5:1)/hexane) to afford 22c (0.35 g) as pale yellow solid. ¹H NMR (400 MHz, CDCl₃, a mixture of rotamers caused by -Boc group) δ 8.58-8.39 (m, 1H), 8.28-8.10 (m, 1H), 7.45-7.27 (m, 1H), 7.25-6.83 (m, 4H), 5.39-5.12 (m, 1H), 4.83-4.64 (m, 1H), 4.20-3.76 (m, 5H), 2.47-2.15 (m, 2H), 1.48-1.25 (m, 9H).

Step 2. Synthesis of 3-(3-fluoro-4-methoxyphenyl)-2-((2R,4S)-4-fluoropyrrolidin-2-yl)-8-(6-methylpyridin-3-yl)-4-oxo-3,4-dihydroquinazoline-6-carbonitrile (25n). A mixture of 22c (350 mg, 0.72 mmol), cesium carbonate (468 mg, 1.44 mmol), 6-methylpyridine-3-boronic acid (108 mg, 0.79 mmol) and tetrakis(triphenylphosphine) palladium (0) (83 mg, 0.072 mmol) in dioxane (5 mL) and H₂O (1 mL) was degassed and flushed with nitrogen. The mixture was heated at 85° C. for 5 h under nitrogen atmosphere. After cooling to room temperature, the mixture was diluted with H₂O (10 mL) and extracted with ethyl acetate (2×20 mL). The combined organic layers were washed with H₂O (10 mL) and brine (10 mL), dried over anhydrous Na₂SO₄, and filtered, then the solvent was evaporated under reduced pressure to afford pale yellow oil. The crude product was treated with a palladium scavenger in triplicate at elevated temperature to remove any residual metal from the coupling reaction. The crude product was separated from the scavenger by filtration and used in the next step. Under nitrogen, a solution of HCl in 1,4-dioxane (3 mL, 4N) was added to a solution of pale yellow oil described above in dry 1,4-dioxane (3 mL) at 0° C., then stirred at room temperature overnight, then the solvent was removed in vacuo and the residue was diluted with CH₂Cl₂ (40 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (70% solvent A (ethyl acetate:MeOH:TEA=100:2:1)/hexane) to afford 25n (30 mg) as white solid. ¹H NMR (400 MHz, CDCl₃) δ 8.74 (d, J=2.3 Hz, 1H), 8.61 (d, J=2.0 Hz, 1H), 7.97 (dd, J=2.0, 0.7 Hz, 1H), 7.80 (dd, J=8.0, 2.4 Hz, 1H), 7.30 (d, J=8.0 Hz, 1H), 7.22-7.09 (m, 2H), 7.05-6.95 (m, 1H), 5.20 (m, 1H), 4.24-4.17 (m, 1H), 3.99 (s, 3H), 3.14-3.04 (m, 2H), 2.66 (s, 3H), 2.34-2.11 (m, 2H). MS (ESI) m/z: 474.1 (M+1)⁺.

### Example 113. Synthesis of (R)-3-(3-fluoro-4-methoxyphenyl)-8-morpholino-4-oxo-2-(pyrrolidin-2-yl)-3,4-dihydroquinazoline-6-carbonitrile (26a)

Prepared as shown in Scheme 8a

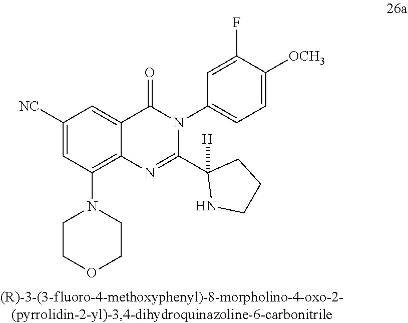 [SMILES: COc1ccc(-n2c([C@H]3CCCN3)nc3c(N4CCOCC4)cc(C#N)cc3c2=O)cc1F]

Step 1. Tris(dibenzylideneacetone)dipalladium (0), Pd₂dba₃ (14 mg, 0.014 mmol) was added to a stirred, degassed, anhydrous solution of BINAP (10 mg, 0.014 mmol), NaOtBu (22 mg, 0.22 mmol), 22a (80 mg, 0.14 mmol) and morpholine (20 □L, 0.22 mmol), in toluene (2 mL). The reaction mixture was stirred at 100° C. for 16 h under argon. The solution was cooled, diluted with EtOAc (3 mL) and washed with saturated aqueous NH₄Cl solution (5 mL), and the aqueous phase was extracted with CH₂Cl₂ (2×3 mL). The combined organic layers were washed with saturated brine (5 mL), dried over anhydrous Na₂SO₄ and concentrated to give crude material which was purified by column chromatography to afford desired Boc-protected intermediate, tert-butyl (R)-2-(6-cyano-3-(3-fluoro-4-methoxyphenyl)-8-morpholino-4-oxo-3,4-dihydroquinazolin-2-yl)pyrrolidine-1-carboxylate, as yellow solid (43 mg, 54%). MS (ESI) m/z: 550 (M+1)⁺.

Step 2. To a solution of tert-butyl (R)-2-(6-cyano-3-(3-fluoro-4-methoxyphenyl)-8-morpholino-4-oxo-3,4-dihydroquinazolin-2-yl)pyrrolidine-1-carboxylate (43 mg) in anhydrous CH₂Cl₂ (3 mL) was added palladium scavenger—SilaMetS-DMT (100 mg), and the mixture was heated to 45° C. for 5 h. The reaction mixture was cooled down, contents were filtered through celite (1 g) using a cintered funnel and the collected material was washed with CH₂Cl₂ (2×3 mL). The filtrate was concentrated in vacuo to obtain solid compound. This palladium scavenging process was repeated three times following the same procedure to obtain Boc-protected intermediate as light-yellow solid (40 mg) which was re-dissolved in anhydrous CH₂Cl₂ (3 mL). Trifluoroacetic acid (0.1 mL) was added at 0° C. The reaction was stirred for 20 h at rt. The resulting mixture was concentrated in vacuo, and the residue was dissolved in 5 mL of CH₂Cl₂ then basified with saturated aqueous Na₂CO₃ solution until the pH=10. The phases were separated, and the aqueous layer was further extracted with CH₂Cl₂ (3×5 mL). The combined organic layer was dried over anhydrous Na₂SO₄, concentrated in vacuo to give a crude material which was purified using column chromatography to afford (R)-3-(3-fluoro-4-methoxyphenyl)-8-morpholino-4-oxo-2-(pyrrolidin-2-yl)-3,4-dihydroquinazoline-6-carbonitrile (26a). ¹H NMR (400 MHz, CDCl₃) δ 8.17 (d, J=1.8 Hz, 1H), 7.32 (dd, J=1.7, 0.9 Hz, 1H), 7.16-6.96 (m, 3H), 3.97 (d, J=6.0 Hz, 8H), 3.37 (dtt, J=15.9, 11.8, 4.2 Hz, 4H), 3.19 (dd, J=10.8, 5.6 Hz, 1H), 2.86 (d, J=10.3 Hz, 1H), 2.49 (s, 1H), 1.89-1.70 (m, 4H). MS (ESI) m/z: 450.1 (M+1)⁺.

### Example 114. 3-(3-fluoro-4-methoxyphenyl)-8-(4-morpholinopiperidine-1-yl)-4-oxo-2-(pyrrolidin-2-yl)-3,4-dihydroquinazoline-6-carbonitrile (26b)

Prepared as shown in Scheme 8a

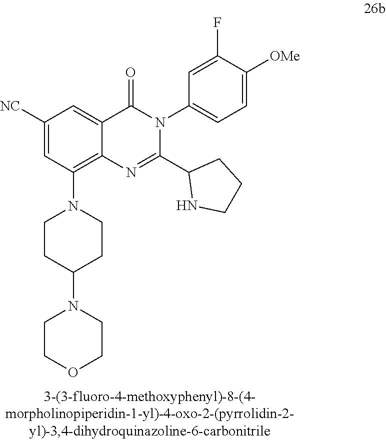 [SMILES: COc1ccc(-n2c(C3CCCN3)nc3c(N4CCC(N5CCOCC5)CC4)cc(C#N)cc3c2=O)cc1F]

### Synthesis of 3-(3-fluoro-4-methoxyphenyl)-8-(4-morpholinopiperidin-1-yl)-4-oxo-2-(pyrrolidin-2-yl)-3,4-dihydroquinazoline-6-carbonitrile (26b)

A mixture of 22d (400 mg, 0.74 mmol), t-BuONa (106 mg, 1.11 mmol), 4-(piperidin-4-yl)morpholine (188 mg, 1.11 mmol), rac-BINAP (46 mg, 0.074 mmol) and tris(dibenzylideneacetone)dipalladium (0) (68 mg, 0.074 mmol) in anhydrous toluene (10 mL) was degassed and flushed with nitrogen. The mixture was heated at 100° C. for 16 h under nitrogen atmosphere. After cooling to room temperature, the mixture was concentrated to remove dioxane, and diluted with H₂O (50 mL) and extracted with DCM (3×60 mL). The combined organic layers were washed with brine (150 mL), dried over anhydrous Na₂SO₄, and filtered, then the solvent was evaporated under reduced pressure to afford a pale-yellow oil. The crude product was purified with flash chromatography (0-3.5% MeOH/DCM) to give the product, which was treated with a palladium scavenger (SiliaMetS DMT, 25 mg), stirred at 40° C. for 8 h and at rt for 16 h, and filtered through Celite pad to remove any residual metal from the coupling reaction. Repeating this procedure of removing Pd for three times. After concentration, the corresponding coupling product (70 mg, Y %=15%) was obtained as a yellow solid. ¹H NMR (400 MHz, Chloroform-d, a mixture of rotamers caused by -Boc group and atropisomers caused by —F atom) δ 8.28-8.09 (m, 1H), 7.45-7.27 (m, 1H), 7.24-7.01 (m, 2H), 7.01-6.88 (m, 1H), 4.59-4.45 (m, 1H), 4.40-4.22 (m, 1H), 4.03-3.92 (m, 3H), 3.83-3.73 (m, 4H), 3.72-3.61 (m, 1H), 3.57-3.46 (m, 1H), 2.99-2.82 (m, 1H), 2.71-2.54 (m, 4H), 2.44-2.27 (m, 1H), 2.28-1.47 (m, 10H), 1.49-1.23 (m, 9H).

Trifluoroacetic acid (0.36 mL, 4.75 mmol) was added to a solution of the corresponding coupling product (100 mg, 0.16 mmol) in DCM (10 mL) at 0° C., then stirred at room temperature overnight, then the solvent was diluted with DCM (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 8.5% MeOH/DCM) to afford 26b (31 mg, 37%) as a yellow solid. ¹H NMR (400 MHz, Chloroform-d, a mixture of two atropisomers (≈1:1 ratio) caused by —F atom) δ 8.14 (s, 1H), 7.33 (s, 1H), 7.14-6.97 (m, 3H), 4.03-3.85 (m, 6H), 3.77 (s, 4H), 3.27-3.16 (m, 1H), 2.90-2.76 (m, 3H), 2.63 (s, 4H), 2.44-2.34 (m, 1H), 2.10-2.02 (m, 2H), 1.86-1.74 (m, 6H). MS (ESI) m/z: 533.0 (M+1)⁺.

### Example 115. Synthesis of (R)-3-(3-fluoro-4-methoxyphenyl)-8-(furan-3-yl)-2-(1-methylpyrrolidin-2-yl)-4-oxo-3,4-dihydroquinazoline-6-carbonitrile (27a)

Prepared as shown in Scheme 8a.

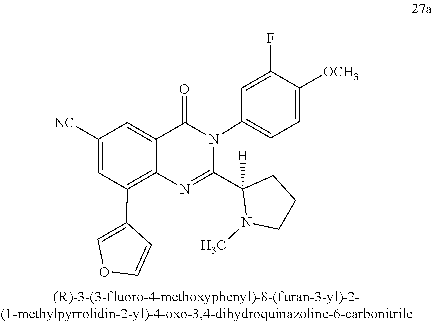 [SMILES: COc1ccc(-n2c([C@H]3CCCN3C)nc3c(-c4ccoc4)cc(C#N)cc3c2=O)cc1F]

Step 1. Synthesis of (R)-8-bromo-3-(3-fluoro-4-methoxyphenyl)-2-(1-methylpyrrolidin-2-yl)-4-oxo-3,4-dihydroquinazoline-6-carbonitrile. A solution of 23a in H₂O (1 mL) was subjected to sequential addition of HCHO (37% aq., 0.1 mL) and HCOOH (0.5 mL). The resulting reaction mixture was heated to 95° C. for 6 h. The reaction was cooled to rt and basified with 1N NaOH until pH-10. A white precipitate crashed out which was extracted with CH₂Cl₂ (3×3 mL). The combined organic layer was dried over anhydrous Na₂SO₄, filtered, concentrated in vacuo to give a crude material which was subjected to column chromatography to afford (R)-8-bromo-3-(3-fluoro-4-methoxyphenyl)-2-(1-methylpyrrolidin-2-yl)-4-oxo-3,4-dihydroquinazoline-6-carbonitrile. MS (ESI) m/z: 457.0 (M+1)⁺.

Step 2. Synthesis of (R)-3-(3-fluoro-4-methoxyphenyl)-8-(furan-3-yl)-2-(1-methylpyrrolidin-2-yl)-4-oxo-3,4-dihydroquinazoline-6-carbonitrile 27a.

Tetrakis(triphenylphosphine)palladium (0), Pd(PPh₃)₄ (10 mg, 0.008 mmol) was added to a stirred, degassed, anhydrous solution of (R)-8-bromo-3-(3-fluoro-4-methoxyphenyl)-2-(1-methylpyrrolidin-2-yl)-4-oxo-3,4-dihydroquinazoline-6-carbonitrile (40 mg, 0.08 mmol), Cs₂CO₃ (57 mg, 0.17 mmol) and furan-3-ylboronic acid (15 mg, 0.13 mmol) in 1,4-dioxane/H₂O (2:1, 4 mL). The reaction mixture was heated to 85° C. for 10 h and then diluted with EtOAc/H₂O (1:1, 8 ml), followed by separation of organic and aqueous phases. The aqueous phase was again extracted with EtOAc (2×4 mL). Combined organic layers were dried over anhydrous Na₂SO₄, concentrated in vacuo and purified by column chromatography (MPLC) to obtain desired (R)-3-(3-fluoro-4-methoxyphenyl)-8-(furan-3-yl)-2-(1-methylpyrrolidin-2-yl)-4-oxo-3,4-dihydroquinazoline-6-carbonitrile as yellow solid (21 mg). The product (21 mg) was dissolved in anhydrous CH₂Cl₂ (3 mL) and combined with palladium scavenger—SilaMetS-DMT (60 mg) and heated to 45° C. for 5 h. The reaction mixture was filtered through celite (1 g) using sintered funnel and washed with CH₂Cl₂ (2×3 mL) to obtain a clear colorless solution. This filtrate was concentrated in vacuo to obtain desired compound, (R)-3-(3-fluoro-4-methoxyphenyl)-8-(furan-3-yl)-2-(1-methylpyrrolidin-2-yl)-4-oxo-3,4-dihydroquinazoline-6-carbonitrile 27a as white solid (16 mg). ¹H NMR (400 MHz, CDCl₃) δ 9.10 (t, J=2.2 Hz, 1H), 8.41 (d, J=1.9 Hz, 1H), 8.08 (d, J=1.9 Hz, 1H), 7.54 (t, J=1.8 Hz, 1H), 7.13 (q, J=8.8 Hz, 1H), 7.07-6.96 (m, 2H), 6.93 (d, J=2.0 Hz, 1H), 3.99 (d, J=1.6 Hz, 3H), 3.19 (qd, J=8.6, 5.5 Hz, 2H), 2.33 (d, J=1.5 Hz, 3H), 2.30-2.20 (m, 1H), 2.02-1.85 (m, 3H), 1.80-1.67 (m, 1H). MS (ESI) m/z: 445.1 (M+1)⁺.

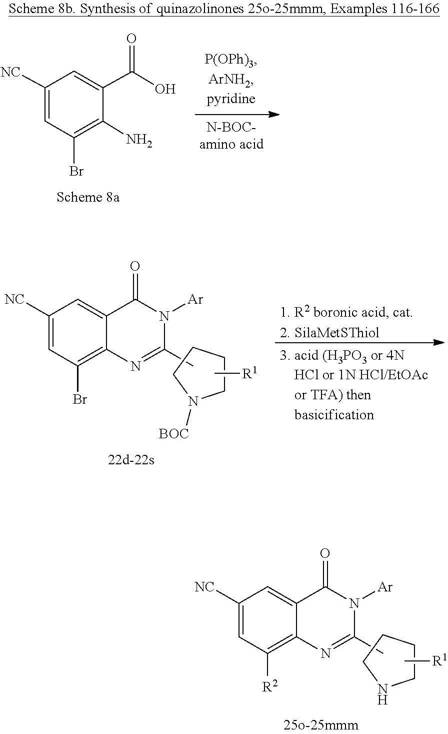

### Example 116. Synthesis of 3-(3-fluoro-4-methoxyphenyl)-2-((2S,4R)-4-hydroxypyrrolidin-2-yl)-8-(6-methylpyridin-3-yl)-4-oxo-3,4-dihydroquinazoline-6-carbonitrile (250)

Prepared as shown in Scheme 8b

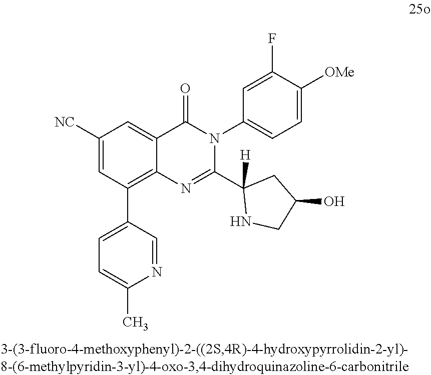 [SMILES: COc1ccc(-n2c([C@@H]3C[C@@H](O)CN3)nc3c(-c4ccc(C)nc4)cc(C#N)cc3c2=O)cc1F]

### Synthesis of tert-butyl (2S,4R)-2-(8-bromo-6-cyano-3-(3-fluoro-4-methoxyphenyl)-4-oxo-3,4-dihydroquinazolin-2-yl)-4-((tert-butyldimethylsilyl)oxy)pyrrolidine-1-carboxylate (22e)

2-amino-3-bromo-5-cyanobenzoic acid (500 mg, 2.08 mmol) and (2S,4R)-1-(tert-butoxycarbonyl)-4-((tert-butyldimethylsilyl)oxy)pyrrolidine-2-carboxylic acid (935 mg, 2.71 mmol) were dissolved in dry pyridine (8 mL). triphenyl phosphite (1.64 mL, 6.25 mmol) was added to the mixture and the reaction mixture was heated at 130° C. with MWI for 30 min. 3-Fluoro-4-methoxyaniline (353 mg, 2.50 mmol) was added to the mixture and the reaction mixture was heated at 100° C. with MWI for 30 min. Pyridine was removed in vacuo and the residue was diluted with CH₂Cl₂ (100 mL) and washed with 1N HCl aqueous (100 mL). The organic layer was washed with H₂O (50 mL) and brine (50 mL), dried over anhydrous Na₂SO₄, and filtered, then the solvent was evaporated under reduced pressure. The residue was purified by flash chromatography (0% to 13% A (DCM:ethyl acetate=1:4)/Hexane)) yielded 22e (500 mg, 71%) as yellow solid. ¹H NMR (400 MHz, Chloroform-d, a mixture of rotamers caused by -Boc group and atropisomers caused by —F atom) δ 8.80-8.63 (m, 2H), 8.14-7.93 (m, 2H), 7.52-7.34 (m, 2H), 7.32-7.12 (m, 2H), 7.05-6.92 (m, 1H), 4.70-4.49 (m, 1H), 4.48-4.29 (m, 1H), 4.11-3.95 (m, 3H), 3.68-3.53 (m, 1H), 3.39-3.21 (m, 1H), 2.78-2.62 (m, 3H), 2.14-1.90 (m, 2H), 1.44-1.43 (m, 4H), 1.36-1.32 (m, 5H), 0.86-0.73 (m, 9H), 0.07-−0.05 (m, 6H).

### Synthesis of 3-(3-fluoro-4-methoxyphenyl)-2-((2S,4R)-4-hydroxypyrrolidin-2-yl)-8-(6-methylpyridin-3-yl)-4-oxo-3,4-dihydroquinazoline-6-carbonitrile (25o)

A mixture of 22e (480 mg, 0.71 mmol), cesium carbonate (465 mg, 1.43 mmol), 2-methylpyridine-5-boronic acid (108 mg, 0.79 mmol) and tetrakis(tri-phenylphosphine) palladium (0) (83 mg, 0.071 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 30% A (DCM:ethyl acetate=1:4)/Hexane) to give the product, which was treated with a palladium scavenger (SiliaMetS Thiol, 90 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (270 mg, 55%) was obtained as a yellow solid. ¹H NMR (400 MHz, Chloroform-d, a mixture of rotamers caused by -Boc group and atropisomers caused by —F atom) δ 8.80-8.63 (m, 2H), 8.14-7.93 (m, 2H), 7.52-7.34 (m, 2H), 7.32-7.12 (m, 2H), 7.05-6.92 (m, 1H), 4.70-4.49 (m, 1H), 4.48-4.29 (m, 1H), 4.11-3.95 (m, 3H), 3.68-3.53 (m, 1H), 3.39-3.21 (m, 1H), 2.78-2.62 (m, 3H), 2.14-1.90 (m, 2H), 1.44-1.43 (m, 4H), 1.36-1.32 (m, 5H), 0.86-0.73 (m, 9H), 0.07-−0.05 (m, 6H).

A solution of H₃PO₄ in water (1.5 mL, W %=84%) was added to a solution of the corresponding coupling product (200 mg, 0.29 mmol) in THE (5 mL) at 0° C., then stirred at room temperature overnight, then the solvent was diluted with CH₂Cl₂ (50 mL) and washed with saturated solutions of Na₂CO₃ (3×50 mL) and brine (50 mL), dried over anhydrous Na₂SO₄, and filtered, then the solvent was evaporated under reduced pressure. The residue was purified by flash reverse phase (C-18) chromatography (0-30% CH₃CN/H₂O) yielded 25o (60 mg, 44%) as pale-yellow solid. ¹H NMR (400 MHz, Chloroform-d, a mixture of two atropisomers (≈1:1 ratio) caused by —F atom) δ 8.75 (d, J=2.3 Hz, 1H), 8.61 (d, J=2.0 Hz, 1H), 7.97 (d, J=2.0 Hz, 1H), 7.82 (dd, J=8.0, 2.3 Hz, 1H), 7.30 (d, J=8.0 Hz, 1H), 7.19-7.05 (m, 2H), 7.03-6.96 (m, 1H), 4.44 (s, 1H), 4.20 (q, J=7.3 Hz, 1H), 3.98 (s, 3H), 3.17-3.09 (m, 1H), 2.81 (d, J=11.8 Hz, 1H), 2.66 (s, 3H), 2.18-2.07 (m, 1H), 1.83 (d, J=8.2 Hz, 1H). MS (ESI) m/z: 472.1 (M+1)⁺.

### Example 117. Synthesis of (R)-3-(3-fluoro-4-methoxyphenyl)-8-(6-methylpyridin-3-yl)-4-oxo-2-(pyrrolidin-2-ylmethyl)-3,4-dihydroquinazoline-6-carbonitrile (25p)

Prepared as shown in Scheme 8b.

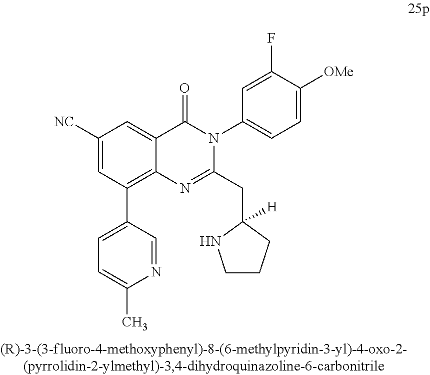 [SMILES: COc1ccc(-n2c(C[C@H]3CCCN3)nc3c(-c4ccc(C)nc4)cc(C#N)cc3c2=O)cc1F]

### Synthesis of tert-butyl (R)-2-((8-bromo-6-cyano-3-(3-fluoro-4-methoxyphenyl)-4-oxo-3,4-dihydroquinazolin-2-yl)methyl)pyrrolidine-1-carboxylate (22f)

The procedure of 22e was followed using 2-amino-3-bromo-5-cyanobenzoic acid (200 mg, 0.83 mmol), (R)-2-(1-(tert-butoxycarbonyl)pyrrolidin-2-yl)acetic acid (249 mg, 1.08 mmol), triphenyl phosphite (0.66 mL, 2.50 mmol) and 3-Fluoro-4-methoxyaniline (141 mg, 1.00 mmol), and pyridine (8 mL). Purification by flash chromatography (0% to 15% A (DCM:ethyl acetate=1:5)/Hexane)) yielded 22f (170 mg, 36%) as pale yellow solid. ¹H NMR (400 MHz, Chloroform-d, a mixture of rotamers caused by -Boc group and atropisomers caused by —F atom) δ 8.51 (s, 1H), 8.18 (s, 1H), 7.42-7.30 (m, 1H), 7.17-7.09 (m, 1H), 7.06-6.93 (m, 1H), 4.33 (s, 1H), 3.98 (d, J=1.7 Hz, 3H), 3.50-3.20 (m, 2H), 3.06-2.85 (m, 1H), 2.47-2.08 (m, 2H), 1.88 (s, 2H), 1.77-1.64 (m, 1H), 1.33-1.08 (m, 9H).

### Synthesis of (R)-3-(3-fluoro-4-methoxyphenyl)-8-(6-methylpyridin-3-yl)-4-oxo-2-(pyrrolidin-2-ylmethyl)-3,4-dihydroquinazoline-6-carbonitrile (25p)

A mixture of 22f (320 mg, 0.58 mmol), cesium carbonate (375 mg, 1.15 mmol), 2-methylpyridine-5-boronic acid (87 mg, 0.63 mmol), tetrakis(tri-phenylphosphine) palladium (0) (66 mg, 0.058 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (20% to 40% A (DCM:ethyl acetate=1:4)/Hexane) to give the product, which was treated with a palladium scavenger (SiliaMetS Thiol, 50 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (160 mg, 49%) was obtained as a yellow solid. ¹H NMR (400 MHz, Chloroform-d, a mixture of rotamers caused by -Boc group and atropisomers caused by —F atom) δ 8.83-8.54 (m, 2H), 8.01-7.81 (m, 2H), 7.72-7.62 (m, 2H), 7.58-7.50 (m, 1H), 7.50-7.42 (m, 2H), 7.43-7.27 (m, 2H), 7.17-6.96 (m, 2H), 4.28-4.11 (m, 1H), 4.02-3.91 (m, 3H), 3.38-3.18 (m, 2H), 2.98-2.72 (m, 1H), 2.66 (s, 3H), 2.46-2.19 (m, 1H), 1.98-1.75 (m, 4H), 1.31-1.21 (m, 9H).

A solution of HCl in 1,4-dioxane (2 mL, 4N, 12 mmol) was added to a solution of the corresponding coupling product (160 mg, 0.28 mmol) in 1,4-dioxane (3 mL) at 0° C., then stirred at room temperature overnight, then the reaction mixture was then adjusted to pH 11 with saturated solutions of Na₂CO₃ and concentrated to remove 1,4-dioxane. The resulting residual was diluted with water (50 mL) and extracted with DCM (3×50 mL). The combined organic layers were washed with brine (100 mL), dried over anhydrous Na₂SO₄, and filtered, then the solvent was evaporated under reduced pressure. The residue was purified by flash chromatography (0-2% MeOH/DCM) to afford 25p (65 mg, 50%) as a pale-yellow solid. ¹H NMR (400 MHz, DMSO-d₆, a mixture of two atropisomers (≈1:1 ratio) caused by —F atom) δ 8.90 (s, 1H), 8.74 (d, J=2.2 Hz, 1H), 8.57 (d, J=1.9 Hz, 1H), 8.38 (d, J=1.9 Hz, 1H), 8.03 (dd, J=8.2, 2.2 Hz, 1H), 7.56-7.24 (m, 4H), 3.94 (s, 3H), 3.85-3.71 (m, 1H), 3.04 (t, J=7.3 Hz, 2H), 3.00-2.87 (m, 1H), 2.79-2.66 (m, 1H), 2.57 (s, 3H), 2.06-1.94 (m, 1H), 1.86-1.69 (m, 2H), 1.59-1.47 (m, 1H). MS (ESI) m/z: 470.0 (M+1)⁺.

### Example 118. Synthesis of (R)-3-(3,5-difluoro-4-methoxyphenyl)-4-oxo-2-(pyrrolidin-2-yl)-8-(6-(trifluoromethyl)pyridin-3-yl)-3,4-dihydroquinazoline-6-carbonitrile (25q)

Prepared as shown in Scheme 8b

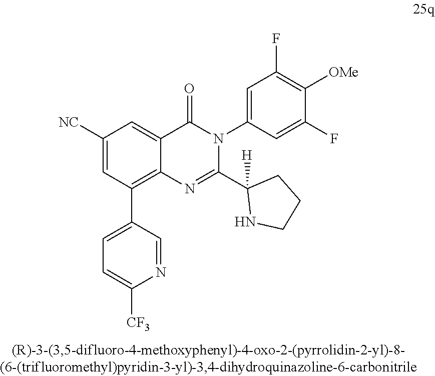 [SMILES: COc1c(F)cc(-n2c([C@H]3CCCN3)nc3c(-c4ccc(C(F)(F)F)nc4)cc(C#N)cc3c2=O)cc1F]

### Synthesis of tert-butyl (R)-2-(8-bromo-6-cyano-3-(3,5-difluoro-4-methoxyphenyl)-4-oxo-3,4-dihydroquinazolin-2-yl)pyrrolidine-1-carboxylate (22g)

The procedure of 22e was followed using 2-amino-3-bromo-5-cyanobenzoic acid (400 mg, 1.67 mmol), Boc-D-proline (466 mg, 2.17 mmol), triphenyl phosphite (1.30 mL, 5.01 mmol) and 3,5-difluoro-4-methoxyaniline (319 mg, 2.00 mmol), and pyridine (8 mL). Purification by flash chromatography (0% to 12% A (DCM:ethyl acetate=1:4)/Hexane)) yielded 22g (540 mg, 58%) as yellow solid. ¹H NMR (400 MHz, Chloroform-d, a mixture of rotamers caused by -Boc group) δ 8.55-8.45 (m, 1H), 8.27-8.13 (m, 1H), 7.26-7.19 (m, 1H), 6.88-6.73 (m, 1H), 4.57-4.45 (m, 1H), 4.17-4.06 (m, 3H), 3.98-3.77 (m, 1H), 3.63-3.41 (m, 1H), 2.32-2.03 (m, 2H), 1.96-1.85 (m, 2H), 1.45-1.26 (m, 10H).

### Synthesis of (R)-3-(3,5-difluoro-4-methoxyphenyl)-4-oxo-2-(pyrrolidin-2-yl)-8-(6-(trifluoromethyl)pyridin-3-yl)-3,4-dihydroquinazoline-6-carbonitrile (25q)

A mixture of 22g (230 mg, 0.41 mmol), cesium carbonate (268 mg, 0.82 mmol), (6-(trifluoromethyl)pyridin-3-yl)boronic acid (62 mg, 0.45 mmol) and tetrakis(tri-phenylphosphine) palladium (0) (10 mg, 0.0082 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 20% A (DCM:ethyl acetate=1:4)/Hexane) to give the N-Boc protected coupled product, which was treated with a palladium scavenger (SiliaMetS Thiol, 60 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupled product (190 mg, 74%) was obtained as a yellow solid. ¹H NMR (400 MHz, Chloroform-d, a mixture of rotamers caused by -Boc group) δ 8.97-8.82 (m, 1H), 8.68-8.62 (m, 1H), 8.30-8.09 (m, 1H), 8.09-8.00 (m, 1H), 7.91-7.76 (m, 1H), 7.24-6.94 (m, 1H), 6.89-6.76 (m, 1H), 4.59-4.43 (m, 1H), 4.18-4.05 (m, 3H), 3.45-3.28 (m, 2H), 2.08-1.80 (m, 4H), 1.34-1.30 (m, 9H). A solution of HCl in 1,4-dioxane (2 mL, 4N, 12 mmol) was added to a solution of the corresponding coupling product (190 mg, 0.30 mmol) in 1,4-dioxane (3 mL) at 0° C., then stirred at room temperature overnight, then the reaction mixture was then adjusted to pH 11 with saturated solutions of Na₂CO₃ and concentrated to remove 1,4-dioxane. The resulting residual was diluted with water (50 mL) and extracted with DCM (3×50 mL). The combined organic layers were washed with brine (100 mL), dried over anhydrous Na₂SO₄, and filtered, then the solvent was evaporated under reduced pressure. The residue was purified by flash chromatography (0-10% MeOH/DCM) to afford 25q (83 mg, 52%) as a pale-yellow solid. ¹H NMR (400 MHz, Chloroform-d) δ 8.98 (s, 1H), 8.65 (s, 1H), 8.13 (d, J=8.1 Hz, 1H), 8.03 (s, 1H), 7.85 (d, J=8.2 Hz, 1H), 6.92 (dd, J=31.7, 10.3 Hz, 2H), 4.12 (s, 3H), 3.97-3.82 (m, 1H), 3.09-2.97 (m, 1H), 2.82-2.71 (m, 1H), 2.20 (s, 1H), 1.88-1.71 (m, 4H). MS (ESI) m/z: 527.9 (M+1)⁺.

### Example 119. Synthesis of (R)-3-(3,5-difluoro-4-methoxyphenyl)-8-(6-methylpyridin-3-yl)-4-oxo-2-(pyrrolidin-2-yl)-3,4-dihydroquinazoline-6-carbonitrile (25r)

Prepared as shown in Scheme 8b

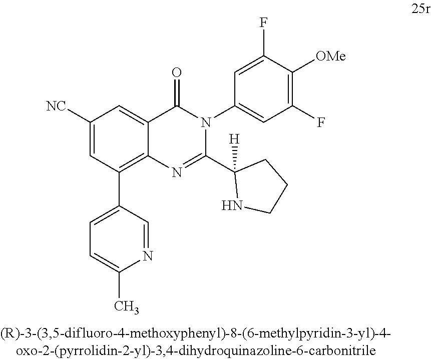 [SMILES: COc1c(F)cc(-n2c([C@H]3CCCN3)nc3c(-c4ccc(C)nc4)cc(C#N)cc3c2=O)cc1F]

### Synthesis of (R)-3-(3,5-difluoro-4-methoxyphenyl)-8-(6-methylpyridin-3-yl)-4-oxo-2-(pyrrolidin-2-yl)-3,4-dihydroquinazoline-6-carbonitrile (25r)

A mixture of 22g (300 mg, 0.54 mmol), cesium carbonate (350 mg, 1.07 mmol), 2-methylpyridine-5-boronic acid (81 mg, 0.59 mmol) and tetrakis(tri-phenylphosphine) palladium (0) (13 mg, 0.011 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 30% A (DCM:ethyl acetate=1:4)/Hexane) to give the product, which was treated with a palladium scavenger (SiliaMetS Thiol, 50 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (160 mg, 52%) was obtained as a yellow solid. ¹H NMR (400 MHz, Chloroform-d, a mixture of rotamers caused by -Boc group) δ 8.66-8.47 (m, 2H), 7.98-7.77 (m, 2H), 7.64-7.54 (m, 4H), 7.51-7.35 (m, 6H), 7.19-7.10 (m, 1H), 6.93-6.69 (m, 1H), 4.49-4.35 (m, 1H), 4.12-3.97 (m, 3H), 3.44-3.19 (m, 2H), 2.65-2.44 (m, 3H), 2.01-1.84 (m, 2H), 1.84-1.71 (m, 2H), 1.27-1.20 (m, 9H).

A solution of HCl in EtOAc (3 mL, 1N, 3 mmol) was added to a solution of the corresponding coupling product (160 mg, 0.28 mmol) in EtOAc (3 mL) at 0° C., then stirred at room temperature overnight, then the reaction mixture was then adjusted to pH 11 with saturated solutions of Na₂CO₃ and concentrated to remove EtOAc. The resulting residual was diluted with water (50 mL) and extracted with DCM (3×50 mL). The combined organic layers were washed with brine (100 mL), dried over anhydrous Na₂SO₄, and filtered, then the solvent was evaporated under reduced pressure. The residue was purified by flash chromatography (0-5% MeOH/DCM) to afford 25r (70 mg, 53%) as a pale-yellow solid. ¹H NMR (400 MHz, Chloroform-d) δ 8.74 (s, 1H), 8.59 (s, 1H), 7.99 (s, 1H), 7.84 (d, J=8.0 Hz, 1H), 7.30 (d, J=8.1 Hz, 1H), 6.91 (dd, J=26.8, 10.3 Hz, 2H), 4.12 (s, 3H), 4.00-3.76 (m, 1H), 3.21-2.96 (m, 1H), 2.87-2.72 (m, 1H), 2.67 (s, 3H), 2.19 (s, 1H), 1.92-1.64 (m, 4H). MS (ESI) m/z: 474.1 (M+1)⁺.

### Example 120. Synthesis of 3-(3-fluoro-4-methoxyphenyl)-2-((4R)-4-methoxypyrrolidin-2-yl)-8-(6-methylpyridin-3-yl)-4-oxo-3,4-dihydroquinazoline-6-carbonitrile (25s)

Prepared as shown in Scheme 8b

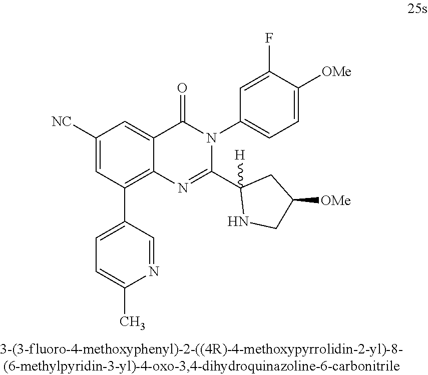 [SMILES: COc1ccc(-n2c(C3C[C@@H](OC)CN3)nc3c(-c4ccc(C)nc4)cc(C#N)cc3c2=O)cc1F]

### Synthesis of tert-butyl (4R)-2-(8-bromo-6-cyano-3-(3-fluoro-4-methoxyphenyl)-4-oxo-3,4-dihydroquinazolin-2-yl)-4-methoxypyrrolidine-1-carboxylate (22h)

The procedure of 22e was followed using 2-amino-3-bromo-5-cyanobenzoic acid (500 mg, 2.08 mmol), (2S,4R)-1-(tert-butoxycarbonyl)-4-methoxypyrrolidine-2-carboxylic acid (663 mg, 2.71 mmol), triphenyl phosphite (1.64 mL, 6.24 mmol) and 3-Fluoro-4-methoxyaniline (352 mg, 2.50 mmol), and pyridine (8 mL). Purification by flash chromatography (0% to 30% A (DCM:ethyl acetate=1:4)/Hexane)) yielded 22 h (620 mg, 52%) as a pale-yellow solid. ¹H NMR (400 MHz, Chloroform-d, a mixture of two diastereomers) δ 8.54-8.45 (m, 1H), 8.25-8.14 (m, 1H), 7.42-7.28 (m, 1H), 7.20-7.05 (m, 2H), 7.02-6.89 (m, 1H), 4.71-4.42 (m, 1H), 4.20-4.01 (m, 1H), 4.01-3.94 (m, 3H), 3.93-3.66 (m, 2H), 3.37-3.21 (m, 3H), 2.34-1.87 (m, 2H), 1.45-1.25 (m, 9H).

### Synthesis of 3-(3-fluoro-4-methoxyphenyl)-2-((4R)-4-methoxypyrrolidin-2-yl)-8-(6-methylpyridin-3-yl)-4-oxo-3,4-dihydroquinazoline-6-carbonitrile (25s)

A mixture of 22h (560 mg, 0.98 mmol), cesium carbonate (638 mg, 1.96 mmol), 2-methylpyridine-5-boronic acid (148 mg, 1.08 mmol) and tetrakis(tri-phenylphosphine) palladium (0) (23 mg, 0.020 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 70% A (DCM:ethyl acetate=1:4)/Hexane) to give the product, which was treated with a palladium scavenger (SiliaMetS Thiol, 110 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (340 mg, 59%) was obtained as a yellow solid. ¹H NMR (400 MHz, Chloroform-d, a mixture of rotamers caused by -Boc group and atropisomers caused by —F atom) δ 8.76-8.56 (m, 2H), 8.06-7.85 (m, 2H), 7.69-7.51 (m, 1H), 7.51-7.36 (m, 1H), 7.36-7.27 (m, 1H), 7.19-6.89 (m, 2H), 4.67-4.45 (m, 1H), 4.07-3.94 (m, 3H), 3.93-3.71 (m, 1H), 3.50-3.37 (m, 1H), 3.28-3.08 (m, 3H), 2.72-2.55 (m, 3H), 2.24-1.87 (m, 2H), 1.35-1.22 (m, 9H).

A solution of HCl in EtOAc (10 mL, 1N, 10 mmol) was added to a solution of the corresponding coupling product (350 mg, 0.60 mmol) in EtOAc (3 mL) at 0° C., then stirred at room temperature overnight, then the reaction mixture was then adjusted to pH 11 with saturated solutions of Na₂CO₃ and concentrated to remove EtOAc. The resulting residual was diluted with water (50 mL) and extracted with DCM (3×50 mL). The combined organic layers were washed with brine (100 mL), dried over anhydrous Na₂SO₄, and filtered, then the solvent was evaporated under reduced pressure. The residue was purified by flash chromatography (0-4% MeOH/DCM) to afford 25s (140 mg, 48%) as a pale-yellow solid. ¹H NMR (400 MHz, Chloroform-d, a mixture of two diastereomers) δ 8.79-8.71 (m, 1H), 8.62-8.56 (m, 1H), 8.01-7.81 (m, 2H), 7.32-7.27 (m, 1H), 7.16-6.97 (m, 3H), 4.10 (t, J=7.5 Hz, 1H), 3.94-3.84 (m, 1H), 3.24-3.18 (m, 3H), 3.10-2.96 (m, 1H), 2.92-2.82 (m, 1H), 2.71-2.64 (m, 3H), 2.18-2.03 (m, 1H), 1.96-1.89 (m, 1H). MS (ESI) m/z: 486.0 (M+1)⁺.

### Example 121. Synthesis of (R)-3-(3-fluoro-4-methoxyphenyl)-8-(6-methylpyridin-3-yl)-4-oxo-2-(5-oxopyrrolidin-2-yl)-3,4-dihydroquinazoline-6-carbonitrile (25t)

Prepared as shown in Scheme 8b.

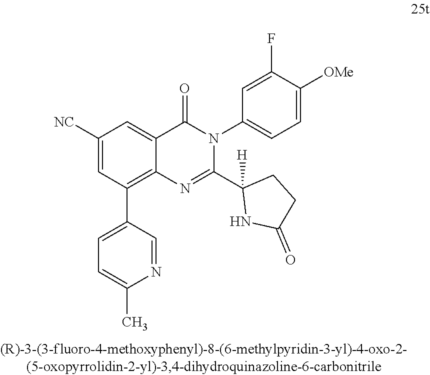 [SMILES: COc1ccc(-n2c([C@H]3CCC(=O)N3)nc3c(-c4ccc(C)nc4)cc(C#N)cc3c2=O)cc1F]

### Synthesis of tert-butyl (R)-2-(8-bromo-6-cyano-3-(3-fluoro-4-methoxyphenyl)-4-oxo-3,4-dihydroquinazolin-2-yl)-5-oxopyrrolidine-1-carboxylate (22i)

The procedure of 22e was followed using 2-amino-3-bromo-5-cyanobenzoic acid (400 mg, 1.67 mmol), (R)-1-(tert-butoxycarbonyl)-5-oxopyrrolidine-2-carboxylic acid (496 mg, 2.17 mmol), triphenyl phosphite (1.31 mL, 5.01 mmol) and 3-Fluoro-4-methoxyaniline (282 mg, 2.00 mmol), and pyridine (8 mL). Purification by flash chromatography (0% to 30% A (DCM:ethyl acetate=1:1)/Hexane)) yielded 22i (500 mg, 71%) as pale yellow solid. ¹H NMR (400 MHz, Chloroform-d, a mixture of rotamers caused by -Boc group and atropisomers caused by —F atom) δ 8.54-8.47 (m, 1H), 8.24-8.16 (m, 1H), 7.25-6.97 (m, 5H), 4.94-4.82 (m, 1H), 4.05-3.93 (m, 3H), 2.98-2.82 (m, 1H), 2.55-2.41 (m, 1H), 2.19-2.05 (m, 1H), 2.02-1.88 (m, 1H), 1.48-1.45 (m, 9H).

### Synthesis of (R)-3-(3-fluoro-4-methoxyphenyl)-8-(6-methylpyridin-3-yl)-4-oxo-2-(5-oxopyrrolidin-2-yl)-3,4-dihydroquinazoline-6-carbonitrile (25t)

A mixture of 22i (510 mg, 0.92 mmol), cesium carbonate (597 mg, 1.83 mmol), 2-methylpyridine-5-boronic acid (138 mg, 1.01 mmol) and tetrakis(tri-phenylphosphine) palladium (0) (79 mg, 0.069 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 95% A (DCM:ethyl acetate=1:4)/Hexane) to give the product, which was treated with a palladium scavenger (SiliaMetS Thiol, 70 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (220 mg, 42%) was obtained as a yellow solid. ¹H NMR (400 MHz, Chloroform-d, a mixture of rotamers caused by -Boc group and atropisomers caused by —F atom) δ 8.61 (d, J=1.9 Hz, 1H), 8.52 (d, J=2.3 Hz, 1H), 8.03 (d, J=1.9 Hz, 1H), 7.97-7.87 (m, 1H), 7.29 (d, J=8.1 Hz, 2H), 7.26-7.19 (m, 1H), 7.19-7.11 (m, 1H), 7.07-6.98 (m, 1H), 4.97-4.79 (m, 1H), 4.01-3.95 (m, 4H), 2.65 (s, 3H), 2.59-2.44 (m, 1H), 2.44-2.28 (m, 1H), 2.09-2.01 (m, 1H), 1.95-1.86 (m, 1H), 1.41-1.35 (m, 9H). A solution of HCl in EtOAc (8 mL, 1N, 8 mmol) was added to a solution of the corresponding coupling product (220 mg, 0.39 mmol) in EtOAc (3 mL) at 0° C., then stirred at room temperature overnight, then the reaction mixture was then adjusted to pH 11 with saturated solutions of Na₂CO₃ and concentrated to remove EtOAc. The resulting residual was diluted with water (50 mL) and extracted with DCM (3×50 mL). The combined organic layers were washed with brine (100 mL), dried over anhydrous Na₂SO₄, and filtered, then the solvent was evaporated under reduced pressure. The residue was purified by flash chromatography (0-7% MeOH/DCM) to afford 25t (120 mg, 66%) as a pale-yellow solid. ¹H NMR (400 MHz, Chloroform-d, a mixture of two atropisomers (≈1:1 ratio) caused by —F atom) δ 8.65 (s, 1H), 8.56 (s, 1H), 7.98 (s, 1H), 7.94-7.80 (m, 1H), 7.28 (s, 1H), 7.22-6.95 (m, 3H), 6.30 (s, 1H), 4.67-4.41 (m, 1H), 3.99 (s, 3H), 2.61 (s, 3H), 2.42-2.05 (m, 4H). MS (ESI) m/z: 470.0 (M+1)⁺.

### Example 122. Synthesis of 3-(3-fluoro-4-methoxyphenyl)-2-((2R,4S)-4-hydroxypyrrolidin-2-yl)-8-(6-methylpyridin-3-yl)-4-oxo-3,4-dihydroquinazoline-6-carbonitrile (25u)

Prepared as shown in Scheme 8b

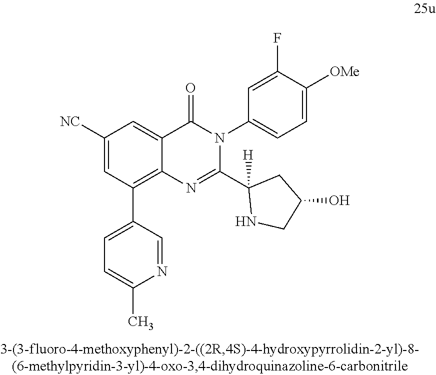 [SMILES: COc1ccc(-n2c([C@H]3C[C@H](O)CN3)nc3c(-c4ccc(C)nc4)cc(C#N)cc3c2=O)cc1F]

### Synthesis of tert-butyl (2R,4S)-2-(8-bromo-6-cyano-3-(3-fluoro-4-methoxyphenyl)-4-oxo-3,4-dihydroquinazolin-2-yl)-4-((tert-butyldimethylsilyl)oxy)pyrrolidine-1-carboxylate (22j)

The procedure of 22e was followed using 2-amino-3-bromo-5-cyanobenzoic acid (1.00 g, 4.17 mmol), (2R,4S)-1-(tert-butoxycarbonyl)-4-((tert-butyldimethylsilyl)oxy)pyrrolidine-2-carboxylic acid (2.00 g, 5.41 mmol), triphenyl phosphite (3.50 mL, 12.51 mmol) and 3-Fluoro-4-methoxyaniline (0.71 g, 5.00 mmol), and pyridine (8 mL). Purification by flash chromatography (0% to 13% A (DCM:ethyl acetate=1:4)/Hexane)) yielded 22j (1.26 g, 45%) as a pink solid. ¹H NMR (400 MHz, Chloroform-d, a mixture of rotamers caused by -Boc group and atropisomers caused by —F atom) δ 8.55-8.45 (m, 1H), 8.25-8.14 (m, 1H), 7.47-7.34 (m, 1H), 7.26-7.05 (m, 7H), 7.04-6.86 (m, 2H), 4.78-4.54 (m, 2H), 4.01-3.94 (m, 3H), 3.94-3.82 (m, 2H), 3.48-3.28 (m, 2H), 2.17-1.88 (m, 3H), 1.42-1.40 (m, 6H), 1.27-1.23 (m, 3H), 0.88 (s, 4H), 0.84-0.80 (m, 9H), 0.14-−0.06 (m, 12H).

### Synthesis of 3-(3-fluoro-4-methoxyphenyl)-2-((2R,4S)-4-hydroxypyrrolidin-2-yl)-8-(6-methylpyridin-3-yl)-4-oxo-3,4-dihydroquinazoline-6-carbonitrile (25u)

A mixture of 22j (640 mg, 0.95 mmol), cesium carbonate (620 mg, 1.90 mmol), 2-methylpyridine-5-boronic acid (170 mg, 1.24 mmol) and tetrakis(tri-phenylphosphine) palladium (0) (83 mg, 0.071 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 50% A (DCM:ethyl acetate=1:4)/Hexane) to give the product, which was treated with a palladium scavenger (SiliaMetS Thiol, 90 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (300 mg, 46%) was obtained as a yellow solid. ¹H NMR (400 MHz, Chloroform-d, a mixture of rotamers caused by -Boc group and atropisomers caused by —F atom) δ 8.72-8.56 (m, 2H), 8.05-7.85 (m, 2H), 7.45-7.29 (m, 1H), 7.26-7.04 (m, 2H), 6.97-6.89 (m, 1H), 4.64-4.45 (m, 1H), 4.33 (dt, J=20.6, 4.9 Hz, 1H), 4.02-3.91 (m, 3H), 3.63-3.47 (m, 1H), 3.33-3.14 (m, 1H), 2.65 (d, J=6.8 Hz, 3H), 2.05-1.83 (m, 2H), 1.44-1.24 (m, 9H), 0.85-0.68 (m, 9H), −0.02-−0.19 (m, 6H).

A solution of HCl in EtOAc (15 mL, 1N, 15 mmol) was added to a solution of the corresponding coupling product (230 mg, 0.34 mmol) in EtOAc (5 mL) at 0° C., then stirred at room temperature overnight, then the reaction mixture was then adjusted to pH 11 with saturated solutions of Na₂CO₃ and concentrated to remove EtOAc. The resulting residual was diluted with water (50 mL) and extracted with DCM (3×50 mL). The combined organic layers were washed with brine (100 mL), dried over anhydrous Na₂SO₄, and filtered, then the solvent was evaporated under reduced pressure. The residue was purified by flash chromatography (0-6% MeOH/DCM) to yield 25u (100 mg, 63%) as a white solid. ¹H NMR (400 MHz, Chloroform-d, a mixture of two atropisomers (≈1:1 ratio) caused by —F atom) δ 8.75 (d, J=2.3 Hz, 1H), 8.60 (d, J=2.0 Hz, 1H), 7.97 (d, J=1.9 Hz, 1H), 7.82 (dd, J=8.0, 2.3 Hz, 1H), 7.30 (d, J=8.0 Hz, 1H), 7.18-7.07 (m, 2H), 7.04-6.94 (m, 1H), 4.44 (s, 1H), 4.20 (q, J=7.3 Hz, 1H), 3.98 (s, 3H), 3.17-3.04 (m, 1H), 2.86-2.75 (m, 1H), 2.66 (s, 3H), 2.23-2.06 (m, 1H), 1.87-1.82 (m, 1H). MS (ESI) m/z: 472.1 (M+1)⁺.

### Example 123. Synthesis of 3-(3-fluoro-4-methoxyphenyl)-4-oxo-2-(pyrrolidin-2-yl)-8-(quinolin-3-yl)-3,4-dihydroquinazoline-6-carbonitrile (25v)

Prepared as shown in Scheme 8b

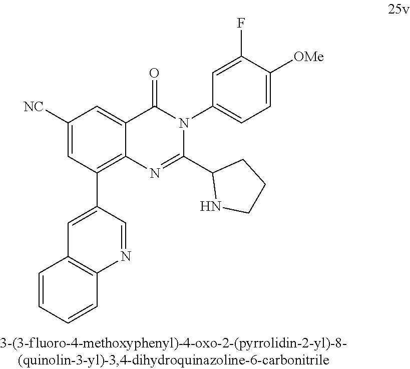 [SMILES: COc1ccc(-n2c(C3CCCN3)nc3c(-c4cnc5ccccc5c4)cc(C#N)cc3c2=O)cc1F]

### Synthesis of 3-(3-fluoro-4-methoxyphenyl)-4-oxo-2-(pyrrolidin-2-yl)-8-(quinolin-3-yl)-3,4-dihydroquinazoline-6-carbonitrile (25v)

A mixture of 22d (500 mg, 0.92 mmol), cesium carbonate (601 mg, 1.84 mmol), quinolin-3-ylboronic acid (207 mg, 1.20 mmol) and tetrakis(tri-phenylphosphine) palladium (0) (80 mg, 0.069 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 50% A (DCM:ethyl acetate=1:4)/Hexane) to give the product, which was treated with a palladium scavenger (SiliaMetS Thiol, 150 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (460 mg, 84%) was obtained as a yellow solid. ¹H NMR (400 MHz, Chloroform-d, a mixture of rotamers caused by -Boc group and atropisomers caused by —F atom) δ 9.34-9.04 (m, 1H), 8.76-8.62 (m, 1H), 8.51-8.37 (m, 1H), 8.33-8.05 (m, 3H), 8.01-7.89 (m, 1H), 7.84-7.76 (m, 1H), 7.73-7.61 (m, 3H), 7.59-7.51 (m, 1H), 7.52-7.42 (m, 2H), 7.42-7.30 (m, 1H), 7.24-6.79 (m, 3H), 4.64-4.42 (m, 1H), 4.06-3.92 (m, 3H), 3.48-3.19 (m, 2H), 2.09-1.91 (m, 2H), 1.91-1.75 (m, 2H), 1.38-1.09 (m, 9H).

A solution of HCl in EtOAc (10 mL, 1N, 10 mmol) was added to a solution of the corresponding coupling product (420 mg, 0.71 mmol) in EtOAc (5 mL) at 0° C., then stirred at room temperature overnight, then the reaction mixture was then adjusted to pH 11 with saturated solutions of Na₂CO₃ and concentrated to remove EtOAc. The resulting residual was diluted with water (50 mL) and extracted with DCM (3×50 mL). The combined organic layers were washed with brine (100 mL), dried over anhydrous Na₂SO₄, and filtered, then the solvent was evaporated under reduced pressure. The residue was purified by flash chromatography (0-5% MeOH/DCM) to yield 25v (200 mg, 57%) as a pale-yellow solid. ¹H NMR (400 MHz, Chloroform-d, a mixture of two atropisomers (≈1:1 ratio) caused by —F atom) δ 9.20 (d, J=2.2 Hz, 1H), 8.69-8.61 (m, 1H), 8.40 (s, 1H), 8.20 (d, J=8.4 Hz, 1H), 8.13 (t, J=1.7 Hz, 1H), 7.93 (d, J=8.2 Hz, 1H), 7.82 (t, J=7.8 Hz, 1H), 7.65 (t, J=7.5 Hz, 1H), 7.16-7.00 (m, 3H), 3.98 (s, 3H), 3.95-3.85 (m, 1H), 3.10-2.98 (m, 1H), 2.78-2.68 (m, 1H), 1.79-1.69 (m, 4H). MS (ESI) m/z: 492.0 (M+1)⁺.

### Example 124. Synthesis of 3-(3-fluoro-4-methoxyphenyl)-8-(6-morpholinopyridin-3-yl)-4-oxo-2-(pyrrolidin-2-yl)-3,4-dihydroquinazoline-6-carbonitrile (25w)

Prepared as shown in Scheme 8b.

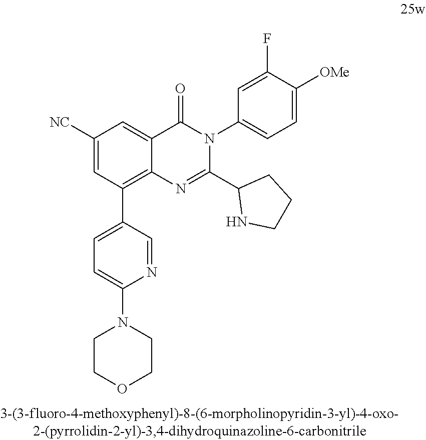 [SMILES: COc1ccc(-n2c(C3CCCN3)nc3c(-c4ccc(N5CCOCC5)nc4)cc(C#N)cc3c2=O)cc1F]

### Synthesis of 3-(3-fluoro-4-methoxyphenyl)-8-(6-morpholinopyridin-3-yl)-4-oxo-2-(pyrrolidin-2-yl)-3,4-dihydroquinazoline-6-carbonitrile (25w)

A mixture of 22d (500 mg, 0.92 mmol), cesium carbonate (601 mg, 1.84 mmol), (6-morpholinopyridin-3-yl)boronic acid (250 mg, 1.20 mmol) and tetrakis(tri-phenylphosphine) palladium (0) (80 mg, 0.069 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 40% A (DCM:ethyl acetate=1:4)/Hexane) to give the product, which was treated with a palladium scavenger (SiliaMetS Thiol, 150 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (460 mg, 83%) was obtained as a yellow solid. ¹H NMR (400 MHz, Chloroform-d, a mixture of rotamers caused by -Boc group and atropisomers caused by —F atom) δ 8.57-8.49 (m, 1H), 8.49-8.30 (m, 1H), 8.08-7.86 (m, 2H), 7.74-7.64 (m, 4H), 7.57-7.33 (m, 7H), 7.25-7.03 (m, 2H), 7.02-6.90 (m, 1H), 6.81-6.67 (m, 1H), 4.64-4.44 (m, 1H), 4.08-3.92 (m, 3H), 3.86 (q, J=4.9 Hz, 4H), 3.72-3.55 (m, 4H), 3.57-3.48 (m, 1H), 3.46-3.33 (m, 1H), 2.03-1.67 (m, 4H), 1.42-1.23 (m, 9H).

A solution of HCl in EtOAc (10 mL, 1N, 10 mmol) was added to a solution of the corresponding coupling product (450 mg, 0.72 mmol) in EtOAc (5 mL) at 0° C., then stirred at room temperature overnight, then the reaction mixture was then adjusted to pH 11 with saturated solutions of Na₂CO₃ and concentrated to remove EtOAc. The resulting residual was diluted with water (50 mL) and extracted with DCM (3×50 mL). The combined organic layers were washed with brine (100 mL), dried over anhydrous Na₂SO₄, and filtered, then the solvent was evaporated under reduced pressure. The residue was purified by flash chromatography (0-5% MeOH/DCM) to yield 25w (130 mg, 34%) as a pale-yellow solid. ¹H NMR (400 MHz, Chloroform-d, a mixture of two atropisomers (≈1:1 ratio) caused by —F atom) δ 8.53 (d, J=1.9 Hz, 1H), 8.44 (d, J=2.4 Hz, 1H), 7.94 (d, J=1.9 Hz, 1H), 7.85 (dt, J=8.8, 2.1 Hz, 1H), 7.16-6.98 (m, 3H), 6.74 (d, J=8.9 Hz, 1H), 3.98 (s, 3H), 3.96-3.90 (m, 1H), 3.87 (t, J=4.9 Hz, 4H), 3.62 (t, J=4.9 Hz, 4H), 3.17-3.08 (m, 1H), 2.82-2.71 (m, 1H), 1.78-1.69 (m, 4H). MS (ESI) m/z: 527.0 (M+1)⁺.

### Example 125. Synthesis of 3-(3-fluoro-4-methoxyphenyl)-8-(6-methoxypyridin-3-yl)-4-oxo-2-(pyrrolidin-2-yl)-3,4-dihydroquinazoline-6-carbonitrile (25x)

Prepared as shown in Scheme 8b.

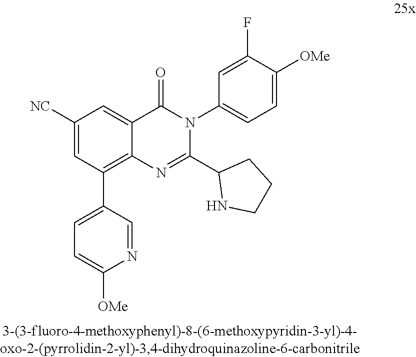 [SMILES: COc1ccc(-c2cc(C#N)cc3c(=O)n(-c4ccc(OC)c(F)c4)c(C4CCCN4)nc23)cn1]

### Synthesis of 3-(3-fluoro-4-methoxyphenyl)-8-(6-methoxypyridin-3-yl)-4-oxo-2-(pyrrolidin-2-yl)-3,4-dihydroquinazoline-6-carbonitrile (25x)

A mixture of 22d (400 mg, 0.74 mmol), cesium carbonate (481 mg, 1.48 mmol), (6-methoxypyridin-3-yl)boronic acid (147 mg, 0.96 mmol) and tetrakis(tri-phenylphosphine) palladium (0) (64 mg, 0.055 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 20% A (DCM:ethyl acetate=1:4)/Hexane) to give the product, which was treated with a palladium scavenger (SiliaMetS Thiol, 80 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (255 mg, 60%) was obtained as a white solid. ¹H NMR (400 MHz, Chloroform-d, a mixture of rotamers caused by -Boc group and atropisomers caused by —F atom) δ 8.62-8.51 (m, 1H), 8.40-8.27 (m, 1H), 8.04-7.90 (m, 2H), 7.43-7.29 (m, 1H), 7.20-7.07 (m, 1H), 7.01-6.81 (m, 2H), 4.66-4.39 (m, 1H), 4.13-3.87 (m, 6H), 3.58-3.23 (m, 2H), 2.08-1.72 (m, 4H), 1.47-1.24 (m, 9H).

A solution of HCl in EtOAc (10 mL, 1N, 10 mmol) was added to a solution of the corresponding coupling product (240 mg, 0.42 mmol) in EtOAc (5 mL) at 0° C., then stirred at room temperature overnight, then the reaction mixture was then adjusted to pH 11 with saturated solutions of Na₂CO₃ and concentrated to remove EtOAc. The resulting residual was diluted with water (50 mL) and extracted with DCM (3×50 mL). The combined organic layers were washed with brine (100 mL), dried over anhydrous Na₂SO₄, and filtered, then the solvent was evaporated under reduced pressure. The residue was purified by flash chromatography (0-3% MeOH/DCM) to yield 25× (145 mg, 73%) as a pale-yellow solid. ¹H NMR (400 MHz, Chloroform-d, a mixture of two atropisomers (≈1:1 ratio) caused by —F atom) δ 8.57 (s, 1H), 8.37 (s, 1H), 7.96 (s, 1H), 7.89 (d, J=8.6 Hz, 1H), 7.18-6.95 (m, 3H), 6.87 (d, J=8.6 Hz, 1H), 4.03 (s, 3H), 3.98 (s, 3H), 3.93-3.83 (m, 1H), 3.18-3.00 (m, 1H), 2.83-2.64 (m, 1H), 2.42-2.08 (m, 2H), 1.85-1.73 (m, 2H). MS (ESI) m/z: 472.0 (M+1)⁺.

### Example 126. Synthesis of 8-(6-cyanopyridin-3-yl)-3-(3-fluoro-4-methoxyphenyl)-4-oxo-2-(pyrrolidin-2-yl)-3,4-dihydroquinazoline-6-carbonitrile (25y)

Prepared as shown in Scheme 8b.

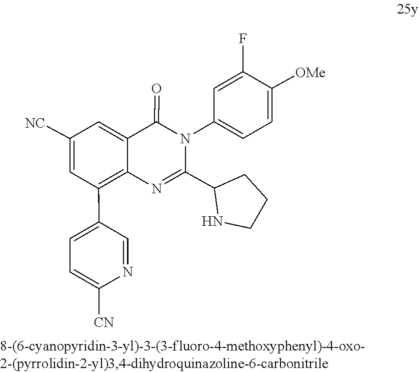 [SMILES: COc1ccc(-n2c(C3CCCN3)nc3c(-c4ccc(C#N)nc4)cc(C#N)cc3c2=O)cc1F]

### Synthesis of 8-(6-cyanopyridin-3-yl)-3-(3-fluoro-4-methoxyphenyl)-4-oxo-2-(pyrrolidin-2-yl)-3,4-dihydroquinazoline-6-carbonitrile (25y)

A mixture of 22d (400 mg, 0.74 mmol), cesium carbonate (481 mg, 1.48 mmol), (6-cyanopyridin-3-yl)boronic acid (142 mg, 0.96 mmol) and tetrakis(tri-phenylphosphine) palladium (0) (64 mg, 0.055 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 40% A (DCM:ethyl acetate=1:4)/Hexane) to give the product, which was treated with a palladium scavenger (SiliaMetS Thiol, 40 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (130 mg, 31%) was obtained as a white solid. ¹H NMR (400 MHz, Chloroform-d, a mixture of rotamers caused by -Boc group and atropisomers caused by —F atom) δ 9.05-8.83 (m, 1H), 8.71-8.55 (m, 1H), 8.27-7.97 (m, 2H), 7.91-7.77 (m, 1H), 7.72-7.41 (m, 1H), 7.42-7.28 (m, 1H), 7.21-7.08 (m, 1H), 7.02-6.89 (m, 1H), 4.61-4.45 (m, 1H), 4.10-3.84 (m, 3H), 3.49-3.24 (m, 2H), 2.14-1.71 (m, 4H), 1.45-1.23 (m, 9H).

A solution of HCl in EtOAc (5 mL, 1N, 5 mmol) was added to a solution of the corresponding coupling product (90 mg, 0.16 mmol) in EtOAc (2 mL) at 0° C., then stirred at room temperature overnight, then the reaction mixture was then adjusted to pH 11 with saturated solutions of Na₂CO₃ and concentrated to remove EtOAc. The resulting residual was diluted with water (50 mL) and extracted with DCM (3×50 mL). The combined organic layers were washed with brine (100 mL), dried over anhydrous Na₂SO₄, and filtered, then the solvent was evaporated under reduced pressure. The residue was purified by flash chromatography (0-5% MeOH/DCM) to yield 25y (35 mg, 47%) as a white solid. ¹H NMR (400 MHz, Chloroform-d, a mixture of two atropisomers (≈1:1 ratio) caused by —F atom) δ 9.10-8.90 (m, 1H), 8.77-8.61 (m, 1H), 8.09 (d, J=8.2 Hz, 1H), 8.01 (s, 1H), 7.86 (d, J=8.1 Hz, 1H), 7.21-6.94 (m, 3H), 3.99 (s, 3H), 3.94-3.85 (m, 1H), 3.10-2.97 (m, 1H), 2.84-2.70 (m, 1H), 1.95-1.75 (m, 4H). MS (ESI) m/z: 467.0 (M+1)⁺.

### Example 127. Synthesis of 3-(3-fluoro-4-methoxyphenyl)-4-oxo-8-(6-(pyrrolidin-1-yl)pyridin-3-yl)-2-(pyrrolidin-2-yl)-3,4-dihydroquinazoline-6-carbonitrile (25z)

Prepared as shown in Scheme 8b.

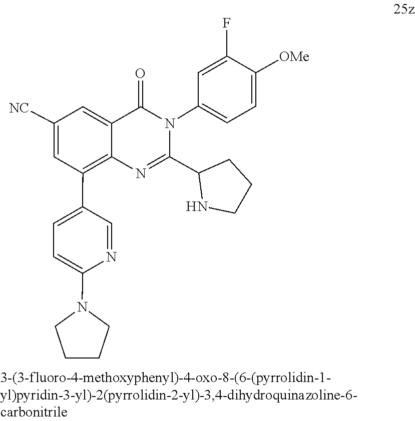 [SMILES: COc1ccc(-n2c(C3CCCN3)nc3c(-c4ccc(N5CCCC5)nc4)cc(C#N)cc3c2=O)cc1F]

### Synthesis of 3-(3-fluoro-4-methoxyphenyl)-4-oxo-8-(6-(pyrrolidin-1-yl)pyridin-3-yl)-2-(pyrrolidin-2-yl)-3,4-dihydroquinazoline-6-carbonitrile (25z)

A mixture of 22d (400 mg, 0.74 mmol), cesium carbonate (481 mg, 1.48 mmol), (6-(pyrrolidin-1-yl)pyridin-3-yl)boronic acid (184 mg, 0.96 mmol) and tetrakis(tri-phenylphosphine) palladium (0) (64 mg, 0.055 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 40% A (DCM:ethyl acetate=1:4)/Hexane) to give the product, which was treated with a palladium scavenger (SiliaMetS Thiol, 120 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (350 mg, 78%) was obtained as a white solid. ¹H NMR (400 MHz, Chloroform-d, a mixture of rotamers caused by -Boc group and atropisomers caused by —F atom) δ 8.53-8.44 (m, 1H), 8.39-8.27 (m, 1H), 8.00-7.83 (m, 2H), 7.74-7.63 (m, 2H), 7.59-7.51 (m, 1H), 7.49-7.31 (m, 3H), 7.20-6.91 (m, 3H), 6.53-6.37 (m, 1H), 4.60-4.45 (m, 1H), 4.05-3.91 (m, 3H), 3.62-3.50 (m, 5H), 3.46-3.32 (m, 1H), 2.10-2.03 (m, 4H), 2.00-1.89 (m, 2H), 1.85-1.71 (m, 2H), 1.42-1.29 (m, 9H).

A solution of HCl in EtOAc (10 mL, 1N, 10 mmol) was added to a solution of the corresponding coupling product (270 mg, 0.44 mmol) in EtOAc (2 mL) at 0° C., then stirred at room temperature overnight, then the reaction mixture was then adjusted to pH 11 with saturated solutions of Na₂CO₃ and concentrated to remove EtOAc. The resulting residual was diluted with water (50 mL) and extracted with DCM (3×50 mL). The combined organic layers were washed with brine (100 mL), dried over anhydrous Na₂SO₄, and filtered, then the solvent was evaporated under reduced pressure. The residue was purified by flash chromatography (0-5% MeOH/DCM) to yield 25z (30 mg, 13%) as a yellow solid. ¹H NMR (400 MHz, Chloroform-d, a mixture of two atropisomers (≈1:1 ratio) caused by —F atom) δ 8.49 (s, 1H), 8.39 (s, 1H), 7.92 (s, 1H), 7.78 (d, J=8.6 Hz, 1H), 7.15-6.98 (m, 3H), 6.46 (d, J=8.8 Hz, 1H), 3.98 (s, 3H), 3.93-3.85 (m, 1H), 3.61-3.47 (m, 4H), 3.21-3.08 (m, 1H), 2.82-2.69 (m, 1H), 2.11-2.05 (m, 4H), 2.02-1.74 (m, 4H). MS (ESI) m/z: 511.0 (M+1)⁺.

### Example 128. Synthesis of (R)-3-(3-fluoro-4-methoxyphenyl)-2-(indolin-2-yl)-8-(6-methylpyridin-3-yl)-4-oxo-3,4-dihydroquinazoline-6-carbonitrile (25aa)

Prepared as shown in Scheme 8b.

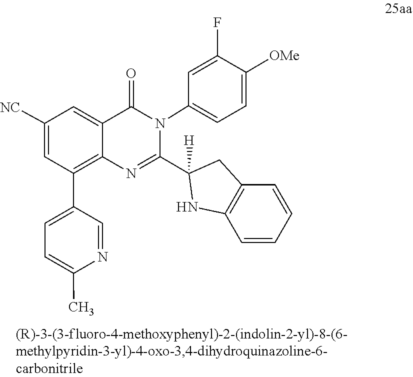 [SMILES: COc1ccc(-n2c([C@H]3Cc4ccccc4N3)nc3c(-c4ccc(C)nc4)cc(C#N)cc3c2=O)cc1F]

### Synthesis of tert-butyl (R)-2-(8-bromo-6-cyano-3-(3-fluoro-4-methoxyphenyl)-4-oxo-3,4-dihydroquinazolin-2-yl)indoline-1-carboxylate (22k)

The procedure of 22d was followed using 2-amino-3-bromo-5-cyanobenzoic acid (1 g, 4.17 mmol), (R)-1-(tert-butoxycarbonyl)indoline-2-carboxylic acid (1.37 g, 5.20 mmol), triphenyl phosphite (3.28 mL, 12.50 mmol) and 3-Fluoro-4-methoxyaniline (706 mg, 5.00 mmol), and pyridine (8 mL). Purification by flash chromatography (0% to 30% A (DCM:ethyl acetate=1:4)/Hexane)) yielded 22k (920 mg, 37%) as yellow solid. ¹H NMR (400 MHz, Chloroform-d, a mixture of rotamers caused by -Boc group and atropisomers caused by —F atom) δ 8.67-8.07 (m, 2H), 7.90-7.63 (m, 1H), 7.62-6.86 (m, 9H), 5.18-5.02 (m, 1H), 3.97-3.69 (m, 3H), 3.48-3.37 (m, 1H), 3.21-3.07 (m, 1H), 1.69-1.20 (m, 9H).

### Synthesis of (R)-3-(3-fluoro-4-methoxyphenyl)-2-(indolin-2-yl)-8-(6-methylpyridin-3-yl)-4-oxo-3,4-dihydroquinazoline-6-carbonitrile (25aa)

A mixture of 22k (900 mg, 1.53 mmol), cesium carbonate (994 mg, 3.05 mmol), 2-methylpyridine-5-boronic acid (272 mg, 1.98 mmol) and tetrakis(tri-phenylphosphine) palladium (0) (132 mg, 0.11 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 40% A (DCM:ethyl acetate=1:4)/Hexane) to give the product, which was treated with a palladium scavenger (SiliaMetS Thiol, 150 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (470 mg, 51%) was obtained as a white solid. ¹H NMR (400 MHz, Chloroform-d, a mixture of rotamers caused by -Boc group and atropisomers caused by —F atom) δ 8.67-8.50 (m, 1H), 8.48-8.34 (m, 1H), 7.98-7.87 (m, 1H), 7.87-7.50 (m, 1H), 7.48-7.28 (m, 2H), 7.25-7.13 (m, 2H), 7.12-6.89 (m, 4H), 5.26-5.06 (m, 1H), 4.08-3.87 (m, 3H), 3.30-3.12 (m, 1H), 3.07-2.90 (m, 1H), 2.74-2.45 (m, 3H), 1.43-1.25 (m, 9H).

A solution of HCl in EtOAc (5 mL, 1N, 5 mmol) was added to a solution of the corresponding coupling product (150 mg, 0.25 mmol) in EtOAc (5 mL) at 0° C., then stirred at room temperature overnight, then the reaction mixture was then adjusted to pH 11 with saturated solutions of Na₂CO₃ and concentrated to remove EtOAc. The resulting residual was diluted with water (50 mL) and extracted with DCM (3×50 mL). The combined organic layers were washed with brine (100 mL), dried over anhydrous Na₂SO₄, and filtered, then the solvent was evaporated under reduced pressure. The residue was purified by flash chromatography (0-5% MeOH/DCM) to yield 25aa (90 mg, 72%) as a yellow solid. ¹H NMR (400 MHz, Chloroform-d, a mixture of two atropisomers (≈1:1 ratio) caused by —F atom) δ 8.60 (s, 2H), 7.96 (s, 1H), 7.64-7.54 (m, 1H), 7.25-6.93 (m, 6H), 6.80-6.67 (m, 1H), 6.67-6.54 (m, 1H), 4.75 (s, 1H), 4.51-4.31 (m, 1H), 4.01 (s, 3H), 3.32-3.16 (m, 1H), 3.14-2.98 (m, 1H), 2.67 (s, 3H). MS (ESI) m/z: 504.0 (M+1)⁺.

### Example 129. Synthesis of 8-(6-acetylpyridin-3-yl)-3-(3-fluoro-4-methoxyphenyl)-4-oxo-2-(pyrrolidin-2-yl)-3,4-dihydroquinazoline-6-carbonitrile (25bb)

Prepared as shown in Scheme 8b.

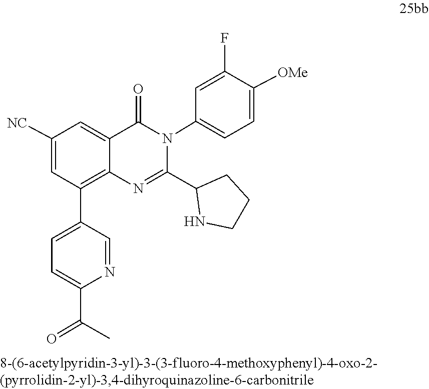 [SMILES: COc1ccc(-n2c(C3CCCN3)nc3c(-c4ccc(C(C)=O)nc4)cc(C#N)cc3c2=O)cc1F]

### Synthesis of 8-(6-acetylpyridin-3-yl)-3-(3-fluoro-4-methoxyphenyl)-4-oxo-2-(pyrrolidin-2-yl)-3,4-dihydroquinazoline-6-carbonitrile (25bb)

A mixture of 22d (400 mg, 0.74 mmol), cesium carbonate (481 mg, 1.48 mmol), 1-(5-(4,4,5,5-tetramethyl-1,3,2-dioxaborolan-2-yl)pyridin-2-yl)ethan-1-one (237 mg, 0.96 mmol) and tetrakis(tri-phenylphosphine) palladium (0) (64 mg, 0.055 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 25% A (DCM:ethyl acetate=1:4)/Hexane) to give the product, which was treated with a palladium scavenger (SiliaMetS Thiol, 120 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (360 mg, 84%) was obtained as a yellow solid. ¹H NMR (400 MHz, Chloroform-d, a mixture of rotamers caused by -Boc group and atropisomers caused by —F atom) δ 8.94-8.80 (m, 1H), 8.70-8.62 (m, 1H), 8.22-8.09 (m, 2H), 8.09-7.99 (m, 1H), 7.76-7.30 (m, 3H), 7.25-6.87 (m, 3H), 4.61-4.43 (m, 1H), 4.03-3.92 (m, 3H), 3.43-3.28 (m, 2H), 2.86-2.74 (m, 3H), 2.04-1.84 (m, 2H), 1.84-1.69 (m, 2H), 1.37-1.23 (m, 9H).

Trifluoroacetic acid (0.87 mL, 11.32 mmol) was added to a solution of the corresponding coupling product (320 mg, 0.57 mmol) in CH₂Cl₂ (10 mL) at 0° C., then stirred at room temperature overnight, then the solvent was diluted with CH₂Cl₂ (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 5% MeOH/CH₂Cl₂) to afford 25bb (150 mg, 55%) as a yellow solid. ¹H NMR (400 MHz, Chloroform-d, a mixture of two atropisomers (≈1:1 ratio) caused by —F atom) δ 8.90 (s, 1H), 8.66 (s, 1H), 8.24-8.15 (m, 1H), 8.15-8.07 (m, 1H), 8.03 (s, 1H), 7.19-6.95 (m, 3H), 3.98 (s, 3H), 3.94-3.84 (m, 1H), 3.11-2.98 (m, 1H), 2.80 (s, 3H), 2.77-2.67 (m, 1H), 1.85-1.62 (m, 4H). MS (ESI) m/z: 484.0 (M+1)⁺.

### Example 130. Synthesis of 8-(6-(dimethylamino)pyridin-3-yl)-3-(3-fluoro-4-methoxyphenyl)-4-oxo-2-(pyrrolidin-2-yl)-3,4-dihydroquinazoline-6-carbonitrile (25cc)

Prepared as shown in Scheme 8b

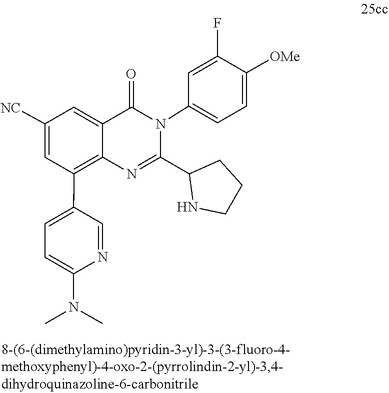 [SMILES: COc1ccc(-n2c(C3CCCN3)nc3c(-c4ccc(N(C)C)nc4)cc(C#N)cc3c2=O)cc1F]

### Synthesis of 8-(6-(dimethylamino)pyridin-3-yl)-3-(3-fluoro-4-methoxyphenyl)-4-oxo-2-(pyrrolidin-2-yl)-3,4-dihydroquinazoline-6-carbonitrile (25cc)

A mixture of 22d (400 mg, 0.74 mmol), cesium carbonate (481 mg, 1.48 mmol), (6-(dimethylamino)pyridin-3-yl)boronic acid (160 mg, 0.96 mmol) and tetrakis(tri-phenylphosphine) palladium (0) (64 mg, 0.055 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 40% A (DCM:ethyl acetate=1:4)/Hexane) to give the product, which was treated with a palladium scavenger (SiliaMetS Thiol, 70 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (200 mg, 47%) was obtained as a yellow solid. ¹H NMR (400 MHz, Chloroform-d, a mixture of rotamers caused by -Boc group and atropisomers caused by —F atom) δ 8.53-8.47 (m, 1H), 8.44-8.27 (m, 1H), 8.02-7.83 (m, 2H), 7.71-7.63 (m, 1H), 7.58-7.42 (m, 2H), 7.40-7.29 (m, 1H), 7.18-7.09 (m, 1H), 7.02-6.91 (m, 1H), 6.66-6.55 (m, 1H), 4.60-4.45 (m, 1H), 4.02-3.93 (m, 3H), 3.63-3.51 (m, 1H), 3.46-3.32 (m, 1H), 3.18 (d, J=3.8 Hz, 6H), 2.02-1.73 (m, 4H), 1.39-1.30 (m, 9H).

Trifluoroacetic acid (0.55 mL, 7.19 mmol) was added to a solution of the corresponding coupling product (210 mg, 0.36 mmol) in DCM (10 mL) at 0° C., then stirred at room temperature overnight, then the solvent was diluted with DCM (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 2.5% MeOH/DCM) to afford 25cc (87 mg, 50%) as a yellow solid. ¹H NMR (400 MHz, Chloroform-d, a mixture of two atropisomers (≈1:1 ratio) caused by —F atom) δ 8.51 (s, 1H), 8.41 (s, 1H), 7.93 (s, 1H), 7.80 (d, J=8.9 Hz, 1H), 7.20-6.96 (m, 3H), 6.61 (d, J=8.8 Hz, 1H), 3.98 (s, 3H), 3.96-3.83 (m, 1H), 3.18 (s, 6H), 3.15-3.07 (m, 1H), 2.82-2.70 (m, 1H), 1.86-1.67 (m, 4H). MS (ESI) m/z: 485.0 (M+1)⁺.

### Example 131. Synthesis of 3-(3-fluoro-4-methoxyphenyl)-8-(6-hydroxypyridin-3-yl)-4-oxo-2-(pyrrolidin-2-yl)-3,4-dihydroquinazoline-6-carbonitrile (25dd)

Prepared as shown in Scheme 8b.

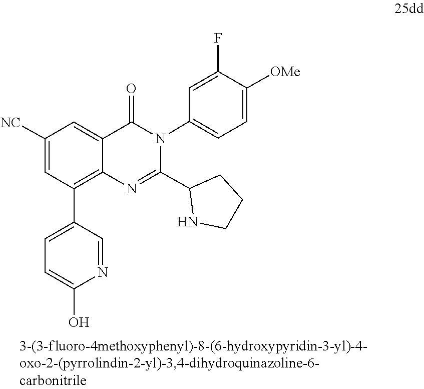 [SMILES: COc1ccc(-n2c(C3CCCN3)nc3c(-c4ccc(O)nc4)cc(C#N)cc3c2=O)cc1F]

### Synthesis of 3-(3-fluoro-4-methoxyphenyl)-8-(6-hydroxypyridin-3-yl)-4-oxo-2-(pyrrolidin-2-yl)-3,4-dihydroquinazoline-6-carbonitrile (25dd)

A mixture of 22d (400 mg, 0.74 mmol), cesium carbonate (481 mg, 1.48 mmol), 6-Hydroxypyridine-3-boronic acid pinacol ester (212 mg, 0.96 mmol) and tetrakis(tri-phenylphosphine) palladium (0) (64 mg, 0.055 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 5% MeOH/DCM) to give the product, which was treated with a palladium scavenger (SiliaMetS Thiol, 70 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (225 mg, 53%) was obtained as a brown solid. ¹H NMR (400 MHz, Chloroform-d, a mixture of rotamers caused by -Boc group and atropisomers caused by —F atom) δ 12.49 (s, 1H), 8.58-8.50 (m, 1H), 7.98-7.93 (m, 1H), 7.92-7.86 (m, 1H), 7.84-7.45 (m, 2H), 7.42-7.31 (m, 1H), 7.24-7.14 (m, 1H), 7.14-7.06 (m, 1H), 7.02-6.92 (m, 1H), 6.75-6.65 (m, 1H), 4.59-4.49 (m, 1H), 4.02-3.95 (m, 3H), 3.62-3.39 (m, 2H), 2.06-1.81 (m, 4H), 1.45-1.30 (m, 9H).

Trifluoroacetic acid (0.58 mL, 7.54 mmol) was added to a solution of the corresponding coupling product (210 mg, 0.38 mmol) in DCM (10 mL) at 0° C., then stirred at room temperature overnight, then the solvent was diluted with DCM (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 5% MeOH/DCM) to afford 25dd (40 mg, 23%) as a white solid. ¹H NMR (400 MHz, Chloroform-d, a mixture of two atropisomers (≈1:1 ratio) caused by —F atom) δ 8.43 (s, 1H), 8.30 (s, 1H), 7.97-7.84 (m, 2H), 7.48 (d, J=11.9 Hz, 1H), 7.42-7.24 (m, 2H), 6.44 (d, J=9.4 Hz, 1H), 3.94 (s, 3H), 3.85-3.74 (m, 1H), 3.03-2.90 (m, 1H), 2.73-2.63 (m, 2H), 2.00-1.88 (m, 1H), 1.76-1.53 (m, 3H). MS (ESI) m/z: 458.0 (M+1)⁺.

### Example 132. Synthesis of 3-(3-fluoro-4-methoxyphenyl)-8-(6-(4-methylpiperazin-1-yl)pyridin-3-yl)-4-oxo-2-(pyrrolidin-2-yl)-3,4-dihydroquinazoline-6-carbonitrile (25ee)

Prepared as shown in Scheme 8b.

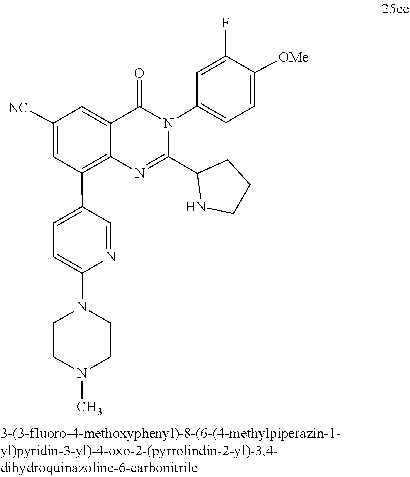 [SMILES: COc1ccc(-n2c(C3CCCN3)nc3c(-c4ccc(N5CCN(C)CC5)nc4)cc(C#N)cc3c2=O)cc1F]

### Synthesis of 3-(3-fluoro-4-methoxyphenyl)-8-(6-(4-methylpiperazin-1-yl)pyridin-3-yl)-4-oxo-2-(pyrrolidin-2-yl)-3,4-dihydroquinazoline-6-carbonitrile (25ee)

A mixture of 22d (300 mg, 0.55 mmol), cesium carbonate (360 mg, 1.11 mmol), (6-(4-methylpiperazin-1-yl)pyridin-3-yl)boronic acid (159 mg, 0.72 mmol) and tetrakis(tri-phenylphosphine) palladium (0) (48 mg, 0.042 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 5% MeOH/DCM) to give the product, which was treated with a palladium scavenger (SiliaMetS Thiol, 70 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (200 mg, 31%) was obtained as a yellow solid. ¹H NMR (400 MHz, Chloroform-d, a mixture of rotamers caused by -Boc group and atropisomers caused by —F atom) δ 8.55-8.48 (m, 1H), 8.46-8.29 (m, 1H), 8.06-7.84 (m, 2H), 7.38-7.31 (m, 1H), 7.20-7.06 (m, 2H), 7.03-6.91 (m, 1H), 6.80-6.67 (m, 1H), 4.62-4.43 (m, 1H), 4.03-3.94 (m, 3H), 3.73-3.62 (m, 4H), 3.60-3.48 (m, 1H), 3.48-3.31 (m, 1H), 2.62-2.51 (m, 4H), 2.38 (s, 3H), 2.10-1.90 (m, 2H), 1.90-1.76 (m, 2H), 1.38-1.30 (m, 9H).

Trifluoroacetic acid (0.68 mL, 8.91 mmol) was added to a solution of the corresponding coupling product (190 mg, 0.30 mmol) in DCM (10 mL) at 0° C., then stirred at room temperature overnight, then the solvent was diluted with DCM (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 10% MeOH/DCM) to afford 25ee (70 mg, 44%) as a yellow solid. ¹H NMR (400 MHz, Chloroform-d, a mixture of two atropisomers (≈1:1 ratio) caused by —F atom) δ 8.52 (d, J=1.7 Hz, 1H), 8.42 (d, J=2.1 Hz, 1H), 7.97-7.89 (m, 1H), 7.83 (d, J=8.8 Hz, 1H), 7.15-6.99 (m, 3H), 6.75 (d, J=8.8 Hz, 1H), 3.98 (s, 3H), 3.94-3.88 (m, 1H), 3.73-3.65 (m, 4H), 3.18-3.09 (m, 1H), 2.80-2.73 (m, 1H), 2.61-2.53 (m, 4H), 2.38 (s, 3H), 1.81-1.69 (m, 4H). MS (ESI) m/z: 540.5 (M+1)⁺.

### Example 133. Synthesis of 3-(3-fluoro-4-methoxyphenyl)-4-oxo-8-(6-phenylpyridin-3-yl)-2-(pyrrolidin-2-yl)-3,4-dihydroquinazoline-6-carbonitrile (25ff)

Prepared as shown in Scheme 8b.

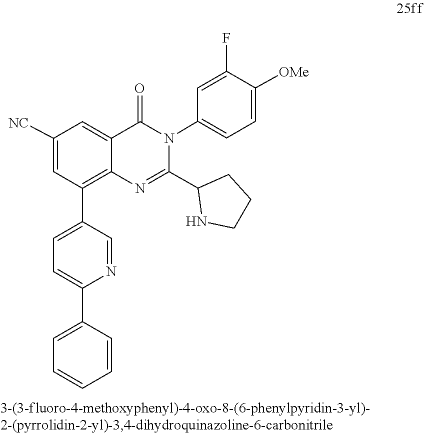 [SMILES: COc1ccc(-n2c(C3CCCN3)nc3c(-c4ccc(-c5ccccc5)nc4)cc(C#N)cc3c2=O)cc1F]

### Synthesis of 3-(3-fluoro-4-methoxyphenyl)-4-oxo-8-(6-phenylpyridin-3-yl)-2-(pyrrolidin-2-yl)-3,4-dihydroquinazoline-6-carbonitrile (25ff)

A mixture of 22d (300 mg, 0.55 mmol), cesium carbonate (360 mg, 1.11 mmol), (6-phenylpyridin-3-yl)boronic acid (143 mg, 0.72 mmol) and tetrakis(tri-phenylphosphine) palladium (0) (48 mg, 0.042 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 5% A (DCM:ethyl acetate=1:4)/Hexane) to give the product, which was treated with a palladium scavenger (SiliaMetS Thiol, 70 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (230 mg, 67%) was obtained as a yellow solid. ¹H NMR (400 MHz, Chloroform-d, a mixture of rotamers caused by -Boc group and atropisomers caused by —F atom) δ 8.94-8.78 (m, 1H), 8.65-8.59 (m, 1H), 8.22-7.84 (m, 5H), 7.56-7.32 (m, 4H), 7.20-6.96 (m, 2H), 4.63-4.43 (m, 1H), 4.02-3.92 (m, 3H), 3.52-3.31 (m, 2H), 2.03-1.74 (m, 4H), 1.37-1.29 (m, 9H).

Trifluoroacetic acid (0.86 mL, 11.17 mmol) was added to a solution of the corresponding coupling product (230 mg, 0.37 mmol) in DCM (10 mL) at 0° C., then stirred at room temperature overnight, then the solvent was diluted with DCM (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 2% MeOH/DCM) to afford 25ff (75 mg, 39%) as a yellow solid. ¹H NMR (400 MHz, Chloroform-d, a mixture of two atropisomers (≈1:1 ratio) caused by —F atom) δ 8.97-8.88 (m, 1H), 8.63 (d, J=1.7 Hz, 1H), 8.14-8.07 (m, 2H), 8.07-8.00 (m, 2H), 7.93-7.85 (m, 1H), 7.57-7.45 (m, 3H), 7.17-7.00 (m, 3H), 3.98 (s, 3H), 3.95-3.85 (m, 1H), 3.12-3.03 (m, 1H), 2.78-2.67 (m, 1H), 1.83-1.66 (m, 4H). MS (ESI) m/z: 518.4 (M+1)⁺.

### Example 134. Synthesis of 3-(3-fluoro-4-methoxyphenyl)-8-(4-(hydroxymethyl)phenyl)-4-oxo-2-(pyrrolidin-2-yl)-3,4-dihydroquinazoline-6-carbonitrile (25gg)

Prepared as shown in Scheme 8b

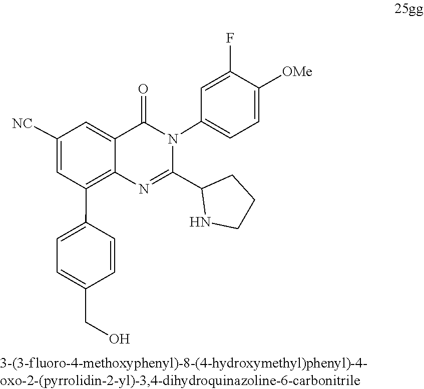 [SMILES: COc1ccc(-n2c(C3CCCN3)nc3c(-c4ccc(CO)cc4)cc(C#N)cc3c2=O)cc1F]

### Synthesis of 3-(3-fluoro-4-methoxyphenyl)-8-(4-(hydroxymethyl)phenyl)-4-oxo-2-(pyrrolidin-2-yl)-3,4-dihydroquinazoline-6-carbonitrile (25gg)

A mixture of 22d (400 mg, 0.74 mmol), cesium carbonate (481 mg, 1.48 mmol), (4-(hydroxymethyl)phenyl)boronic acid (146 mg, 0.96 mmol) and tetrakis(tri-phenylphosphine) palladium (0) (64 mg, 0.055 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 25% A (DCM:ethyl acetate=1:4)/Hexane) to give the product, which was treated with a palladium scavenger (SiliaMetS Thiol, 110 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (330 mg, 78%) was obtained as a white solid. ¹H NMR (400 MHz, Chloroform-d, a mixture of rotamers caused by -Boc group and atropisomers caused by —F atom) δ 8.61-8.53 (m, 1H), 8.05-7.94 (m, 1H), 7.68-7.59 (m, 2H), 7.48 (t, J=8.4 Hz, 2H), 7.40-7.34 (m, 1H), 7.20-7.07 (m, 2H), 7.01-6.92 (m, 1H), 4.88-4.73 (m, 2H), 4.60-4.42 (m, 1H), 4.02-3.94 (m, 3H), 3.45-3.27 (m, 2H), 2.06-1.71 (m, 5H), 1.36-1.27 (m, 9H).

Trifluoroacetic acid (1.33 mL, 17.35 mmol) was added to a solution of the corresponding coupling product (330 mg, 0.58 mmol) in DCM (10 mL) at 0° C., then stirred at room temperature overnight, then the solvent was diluted with DCM (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 4% MeOH/DCM) to afford 25gg (143 mg, 53%) as a yellow solid. ¹H NMR (400 MHz, Chloroform-d, a mixture of two atropisomers (≈1:1 ratio) caused by —F atom) δ 8.57 (s, 1H), 7.98 (s, 1H), 7.67-7.57 (m, 2H), 7.57-7.39 (m, 2H), 7.21-6.91 (m, 3H), 4.80 (s, 2H), 3.98 (s, 3H), 3.95-3.78 (m, 1H), 3.11-2.97 (m, 1H), 2.81-2.63 (m, 1H), 2.29 (s, 1H), 1.86-1.57 (m, 4H). MS (ESI) m/z: 471.7 (M+1)⁺.

### Example 135. Synthesis of 8-(5,6-dimethoxypyridin-3-yl)-3-(3-fluoro-4-methoxyphenyl)-4-oxo-2-(pyrrolidin-2-yl)-3,4-dihydroquinazoline-6-carbonitrile (25 hh)

Prepared as shown in Scheme 8b.

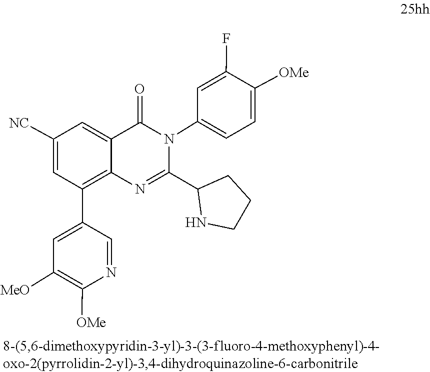 [SMILES: COc1ccc(-n2c(C3CCCN3)nc3c(-c4cnc(OC)c(OC)c4)cc(C#N)cc3c2=O)cc1F]

### Synthesis of 8-(5,6-dimethoxypyridin-3-yl)-3-(3-fluoro-4-methoxyphenyl)-4-oxo-2-(pyrrolidin-2-yl)-3,4-dihydroquinazoline-6-carbonitrile (25 hh)

A mixture of 22d (300 mg, 0.55 mmol), cesium carbonate (360 mg, 1.11 mmol), 2,3-Dimethoxy-5-(4,4,5,5-tetramethyl-1,3,2-dioxaborolan-2-yl)pyridine (191 mg, 0.72 mmol) and tetrakis(tri-phenylphosphine) palladium (0) (48 mg, 0.042 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 15% A (DCM:ethyl acetate=1:4)/Hexane) to give the product, which was treated with a palladium scavenger (SiliaMetS Thiol, 80 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (250 mg, 75%) was obtained as a white solid. ¹H NMR (400 MHz, Chloroform-d, a mixture of rotamers caused by -Boc group and atropisomers caused by —F atom) δ 8.63-8.55 (m, 1H), 8.01-7.79 (m, 2H), 7.70-7.34 (m, 3H), 7.24-7.05 (m, 2H), 7.02-6.88 (m, 1H), 4.61-4.42 (m, 1H), 4.12-3.94 (m, 9H), 3.50-3.33 (m, 2H), 2.05-1.87 (m, 2H), 1.89-1.72 (m, 2H), 1.37-1.25 (m, 9H).

Trifluoroacetic acid (0.91 mL, 12.00 mmol) was added to a solution of the corresponding coupling product (240 mg, 0.40 mmol) in DCM (10 mL) at 0° C., then stirred at room temperature overnight, then the solvent was diluted with DCM (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 3% MeOH/DCM) to afford 25 hh (90 mg, 45%) as a white solid. ¹H NMR (400 MHz, Chloroform-d, a mixture of two atropisomers (≈1:1 ratio) caused by —F atom) δ 8.59 (s, 1H), 7.98 (s, 1H), 7.92 (s, 1H), 7.41 (s, 1H), 7.16-6.99 (m, 3H), 4.11 (s, 3H), 3.98 (s, 3H), 3.94 (s, 3H), 3.91-3.84 (m, 1H), 3.12-3.02 (m, 1H), 2.78-2.69 (m, 1H), 1.81-1.65 (m, 4H). MS (ESI) m/z: 502.6 (M+1)⁺.

### Example 136. Synthesis of 3-(3-fluoro-4-methoxyphenyl)-8-(4-(methylsulfonyl)phenyl)-4-oxo-2-(pyrrolidin-2-yl)-3,4-dihydroquinazoline-6-carbonitrile (25ii)

Prepared as shown in Scheme 8b.

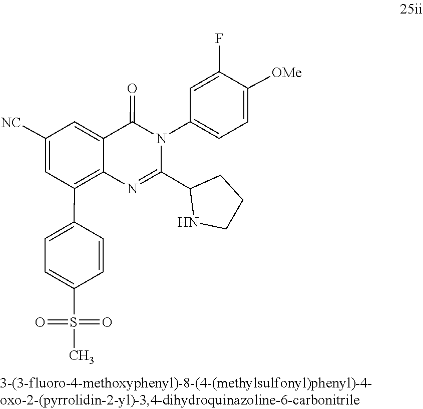 [SMILES: COc1ccc(-n2c(C3CCCN3)nc3c(-c4ccc(S(C)(=O)=O)cc4)cc(C#N)cc3c2=O)cc1F]

### Synthesis of 3-(3-fluoro-4-methoxyphenyl)-8-(4-(methylsulfonyl)phenyl)-4-oxo-2-(pyrrolidin-2-yl)-3,4-dihydroquinazoline-6-carbonitrile (25ii)

A mixture of 22d (400 mg, 0.74 mmol), cesium carbonate (481 mg, 1.48 mmol), (4-(methylsulfonyl)phenyl)boronic acid (192 mg, 0.96 mmol) and tetrakis(tri-phenylphosphine) palladium (0) (64 mg, 0.0552 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 64% A (DCM:ethyl acetate=1:4)/Hexane) to give the product, which was treated with a palladium scavenger (SiliaMetS Thiol, 110 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (340 mg, 81%) was obtained as a white solid. ¹H NMR (400 MHz, Chloroform-d, a mixture of rotamers caused by -Boc group and atropisomers caused by —F atom) δ 8.67-8.59 (m, 1H), 8.11-7.98 (m, 3H), 7.88-7.80 (m, 2H), 7.45-7.29 (m, 1H), 7.20-7.09 (m, 1H), 7.01-6.90 (m, 1H), 4.63-4.40 (m, 1H), 4.04-3.92 (m, 3H), 3.41-3.25 (m, 2H), 3.14 (s, 3H), 2.05-1.70 (m, 4H), 1.39-1.27 (m, 9H). Trifluoroacetic acid (1.15 mL, 15.03 mmol) was added to a solution of the corresponding coupling product (310 mg, 0.50 mmol) in DCM (10 mL) at 0° C., then stirred at room temperature overnight, then the solvent was diluted with DCM (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 9% MeOH/DCM) to afford 25ii (135 mg, 52%) as a white solid. ¹H NMR (400 MHz, Chloroform-d, a mixture of two atropisomers (≈1:1 ratio) caused by —F atom) δ 8.64 (s, 1H), 8.08 (d, J=8.2 Hz, 2H), 8.00 (s, 1H), 7.83 (d, J=8.2 Hz, 2H), 7.18-6.98 (m, 3H), 3.98 (s, 3H), 3.94-3.82 (m, 1H), 3.15 (s, 3H), 3.09-2.98 (m, 1H), 2.85-2.64 (m, 1H), 1.80-1.67 (m, 4H). MS (ESI) m/z: 519.0 (M+1)⁺.

### Example 137. Synthesis of 8-(5,6-dimethylpyridin-3-yl)-3-(3-fluoro-4-methoxyphenyl)-4-oxo-2-(pyrrolidin-2-yl)-3,4-dihydroquinazoline-6-carbonitrile (25jj)

Prepared as shown in Scheme 8b.

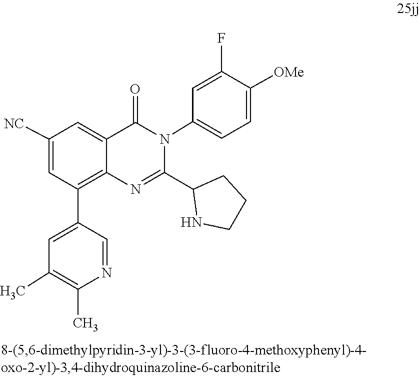 [SMILES: COc1ccc(-n2c(C3CCCN3)nc3c(-c4cnc(C)c(C)c4)cc(C#N)cc3c2=O)cc1F]

### Synthesis of 8-(5,6-dimethylpyridin-3-yl)-3-(3-fluoro-4-methoxyphenyl)-4-oxo-2-(pyrrolidin-2-yl)-3,4-dihydroquinazoline-6-carbonitrile (25jj)

A mixture of 22d (400 mg, 0.74 mmol), cesium carbonate (481 mg, 1.48 mmol), 2,3-dimethyl-5-(4,4,5,5-tetramethyl-1,3,2-dioxaborolan-2-yl)pyridine (223 mg, 0.96 mmol) and tetrakis(tri-phenylphosphine) palladium (0) (64 mg, 0.0552 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 64% A (DCM:ethyl acetate=1:4)/Hexane) to give the product, which was treated with a palladium scavenger (SiliaMetS Thiol, 90 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, the corresponding coupling product (270 mg, 64%) was obtained as a brown solid. ¹H NMR (400 MHz, Chloroform-d, a mixture of rotamers caused by -Boc group and atropisomers caused by —F atom) δ 8.64-8.56 (m, 1H), 8.55-8.38 (m, 1H), 8.04-7.92 (m, 1H), 7.89-7.63 (m, 2H), 7.56-7.31 (m, 2H), 7.25-6.87 (m, 3H), 4.63-4.41 (m, 1H), 4.04-3.91 (m, 3H), 3.50-3.31 (m, 2H), 2.60 (s, 3H), 2.47-2.36 (m, 3H), 2.04-1.80 (m, 4H), 1.33-1.24 (m, 9H).

Trifluoroacetic acid (1.09 mL, 14.22 mmol) was added to a solution of the corresponding coupling product (270 mg, 0.47 mmol) in DCM (10 mL) at 0° C., then stirred at room temperature overnight, then the solvent was diluted with DCM (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 5% MeOH/DCM) to afford 25jj (120 mg, 54%) as a white solid. ¹H NMR (400 MHz, Chloroform-d, a mixture of two atropisomers (≈1:1 ratio) caused by —F atom) δ 8.59 (d, J=1.8 Hz, 1H), 8.57 (d, J=1.7 Hz, 1H), 8.01-7.95 (m, 1H), 7.71 (s, 1H), 7.17-6.98 (m, 3H), 3.98 (s, 3H), 3.95-3.85 (m, 1H), 3.14-3.03 (m, 1H), 2.82-2.71 (m, 1H), 2.61 (s, 3H), 2.39 (s, 3H), 1.84-1.67 (m, 4H). MS (ESI) m/z: 470.0 (M+1)⁺.

### Example 138. Synthesis of 3-(3-fluoro-4-methoxyphenyl)-8-(2-methylpyridin-4-yl)-4-oxo-2-(pyrrolidin-2-yl)-3,4-dihydroquinazoline-6-carbonitrile (25kk)

Prepared as shown in Scheme 8b.

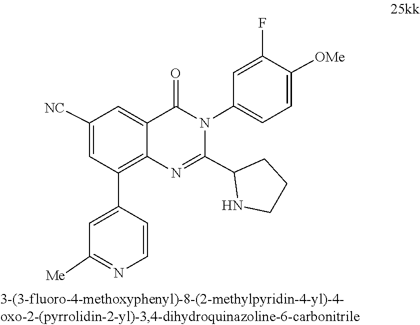 [SMILES: COc1ccc(-n2c(C3CCCN3)nc3c(-c4ccnc(C)c4)cc(C#N)cc3c2=O)cc1F]

### Synthesis of tert-butyl 2-(6-cyano-3-(3-fluoro-4-methoxyphenyl)-8-(2-methylpyridin-4-yl)-4-oxo-3,4-dihydroquinazolin-2-yl) pyrrolidine-1-carboxylate

A microwave vial with stir bar was charged with 22d (200 mg, 0.37 mmol, 1 equiv.), boronic acid (56 mg, 0.41 mmol, 1.1 equiv.), Cs₂CO₃ (241 mg, 0.74 mmol, 2 equiv.), and Pd(PPh₃)₄ (43 mg, 0.037 mmol, 0.1 equiv.). A 4:1 mixture of 1,4-dioxane:H₂O was added via syringe (5.25 mL). The vial was sealed with a septum and the mixture was sparged with N₂ for 15 min. After 15 min, the reaction was heated to 80° C. for 17 h. After 17 h, the reaction was cooled to room temperature and diluted with EtOAc and H₂O (10 mL each). The layers were shaken and separated. The aqueous phase was washed twice with EtOAc (10 mL each). The combined organics were dried over Na₂SO₄, filtered, and concentrated. The crude reaction mixture was purified via silica gel chromatography (3:1 to 1:1 hexanes:EtOAc). Trace palladium was removed by dissolving the purified product in DCM (0.2 M) and adding SiliametS Thiol Metal Scavenger (33 wt %) to the solution. The mixture was heated to 40° C. for 8 h before being filtered through celite. This procedure was repeated two more times. tert-butyl 2-(6-cyano-3-(3-fluoro-4-methoxyphenyl)-8-(2-methylpyridin-4-yl)-4-oxo-3,4-dihydroquinazolin-2-yl) pyrrolidine-1-carboxylate obtained as a mixture contaminated with triphenylphosphine oxide and was used directly in the next step.

### Synthesis of 3-(3-fluoro-4-methoxyphenyl)-8-(2-methylpyridin-4-yl)-4-oxo-2-(pyrrolidin-2-yl)-3,4-dihydroquinazoline-6-carbonitrile (25kk)

To a vial with stir bar was added tert-butyl 2-(6-cyano-3-(3-fluoro-4-methoxyphenyl)-8-(2-methylpyridin-4-yl)-4-oxo-3,4-dihydroquinazolin-2-yl) pyrrolidine-1-carboxylate (175 mg, 0.31 mmol, 1 equiv.) and 4N HCl in 1,4-dioxane (0.6 mL). The reaction was allowed to stir at room temperature for 1 hour. The solvent was removed in vacuo and the crude residue was dissolved in DCM (5 mL). The residual HCl was quenched with sat. aq. NaHCO₃ (5 mL).

The layers were shaken and separated and the aqueous phase was washed with DCM (2×, 5 mL). The combined organics were dried over Na₂SO₄, filtered, and concentrated. The crude reaction mixture was purified via silica gel chromatography (0 to 5% 4:1 (MeOH:sat. aq. NH₄OH in DCM)). The product, 3-(3-fluoro-4-methoxyphenyl)-8-(2-methylpyridin-4-yl)-4-oxo-2-(pyrrolidin-2-yl)-3,4-dihydroquinazoline-6-carbonitrile, (25kk) was obtained as a white foam (109 mg, 0.24 mmol, 64% yield over 2 steps). ¹H NMR (400 MHz, CDCl₃) δ 8.63 (d, J=5.1 Hz, 2H), 8.00 (s, 1H), 7.42 (s, 1H), 7.35 (d, J=4.8 Hz, 1H), 7.19-6.96 (m, 3H), 3.98 (s, 3H), 3.91 (q, J=7.5 Hz, 1H), 3.06 (dd, J=11.3, 6.0 Hz, 1H), 2.76 (dt, J=11.4, 6.5 Hz, 1H), 2.67 (s, 3H), 2.24 (s, 2H), 1.76-1.69 (m, 2H). MS (ESI) m/z: 457.0 (M+1)⁺.

### Example 139. Synthesis of 3-(3-fluoro-4-methoxyphenyl)-8-(6-methylpyridin-3-yl)-2-((2S,3aS,7aS)-octahydro-1H-indol-2-yl)-4-oxo-3,4-dihydroquinazoline-6-carbonitrile (25ll)

Prepared as shown in Scheme 8b.

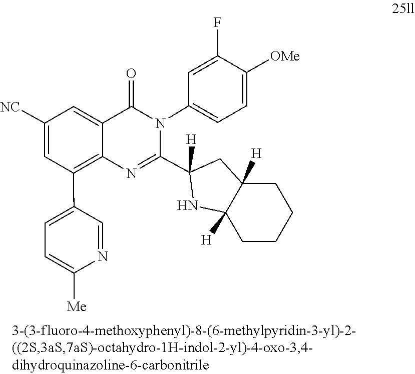 [SMILES: COc1ccc(-n2c([C@@H]3C[C@@H]4CCCC[C@@H]4N3)nc3c(-c4ccc(C)nc4)cc(C#N)cc3c2=O)cc1F]

### Synthesis of tert-butyl (2S,3aS,7aS)-2-(8-bromo-6-cyano-3-(3-fluoro-4-methoxyphenyl)-4-oxo-3,4-dihydroquinazolin-2-yl)octahydro-1H-indole-1-carboxylate (22o)

A microwave vial with stir bar was charged 2-amino-3-bromo-5-cyanobenzoic acid (275 mg, 1.14 mmol, 1 equiv.), (2S,3aS,7aS)-1-(tert-butoxycarbonyl)octahydro-1H-indole-2-carboxylic acid (400 mg, 1.49 mmol, 1.3 equiv.), P(OPh)₃ (0.9 mL, 3.42 mmol, 3 equiv.), and pyridine (2.85 mL). The vial was sealed and heated under microwave irradiation at 130° C. for 40 min. After 40 min, the reaction was cooled and 3-fluoro-4-methoxyaniline (193 mg, 1.37 mmol, 1.2 equiv.) was added in one portion. The vial was sealed and heated under microwave irradiation at 130° C. for 40 min. The reaction was cooled to r.t. and diluted with DCM (20 mL). The reaction mixture was washed with 1N HCl (20 mL), 1N NaOH (20 mL), and brine (20 mL). The organic phase was dried over MgSO₄, filtered, and concentrated. Purified twice by column chromatography (1^{st} column, 0 to 5% EtOAc in DCM, 2^{nd} column 3:1 hexanes:EtOAc). tert-butyl (2S,3aS,7aS)-2-(8-bromo-6-cyano-3-(3-fluoro-4-methoxyphenyl)-4-oxo-3,4-dihydroquinazolin-2-yl)octahydro-1H-indole-1-carboxylate (22o) obtained as a white foam (465 mg, 0.78 mmol, 68% yield). (mixture of rotamers)¹H NMR (400 MHz, CDCl₃) δ 8.50 (m, 1H), 8.28-8.06 (m, 1H), 7.42-7.30 (m, 1H), 7.20-7.04 (m, 2H), 7.03-6.88 (m, 1H), 4.61-4.34 (m, 1H), 3.98 (m, 3H), 3.93-3.72 (m, 1H), 2.46-2.05 (m, 4H), 1.94-1.54 (m, 6H), 1.53-1.28 (m, 10H).

A microwave vial with stir bar was charged with bromide 22o (200 mg, 0.33 mmol, 1 equiv.), boronic acid (50 mg, 0.37 mmol, 1.1 equiv.), Cs₂CO₃ (215 mg, 0.66 mmol, 2 equiv.), and Pd(PPh₃)₄ (38 mg, 0.033 mmol, 0.1 equiv.). A 4:1 mixture of 1,4-dioxane:H₂O was added via syringe (4.11 mL). The vial was sealed with a septum and the mixture was sparged with N₂ for 15 min. After 15 min, the reaction was heated to 80° C. for 17 h. After 17 h, the reaction was cooled to room temperature and diluted with EtOAc and H₂O (10 mL each). The layers were shaken and separated. The aqueous phase was washed twice with EtOAc (10 mL each). The combined organics were dried over Na₂SO₄, filtered, and concentrated. The crude reaction mixture was purified via silica gel chromatography (3:1 to 1:1 hexanes:EtOAc). Trace palladium was removed by dissolving the purified product tert-butyl (2S,3aS,7aS)-2-(6-cyano-3-(3-fluoro-4-methoxyphenyl)-8-(6-methylpyridin-3-yl)-4-oxo-3,4-dihydroquinazolin-2-yl)octahydro-1H-indole-1-carboxylate in DCM (0.2 M) and adding SiliametS Thiol Metal Scavenger (33 wt %) to the solution. The mixture was heated to 40° C. for 8 h before being filtered through celite. This procedure was repeated two more times. tert-butyl (2S,3aS,7aS)-2-(6-cyano-3-(3-fluoro-4-methoxyphenyl)-8-(6-methylpyridin-3-yl)-4-oxo-3,4-dihydroquinazolin-2-yl)octahydro-1H-indole-1-carboxylate obtained as a white foam (124 mg, 0.2 mmol, 61% yield). (Mixture of rotamers) 1H NMR (400 MHz, Chloroform-d) δ 8.72-8.53 (m, 2H), 8.07-7.74 (m, 2H), 7.45-7.29 (m, 1H), 7.25-6.88 (m, 3H), 4.62-4.29 (m, 1H), 4.04-3.93 (m, 3H), 3.86-3.50 (m, 1H), 2.65 (dd, J=17.0, 7.7 Hz, 3H), 2.23-1.69 (m, 3H), 1.46-0.81 (m, 17H).

### Synthesis of 3-(3-fluoro-4-methoxyphenyl)-8-(6-methylpyridin-3-yl)-2-((2S,3aS,7aS)-octahydro-1H-indol-2-yl)-4-oxo-3,4-dihydroquinazoline-6-carbonitrile (25ll)

To a vial with stir bar was added tert-butyl (2S,3aS,7aS)-2-(6-cyano-3-(3-fluoro-4-methoxyphenyl)-8-(6-methylpyridin-3-yl)-4-oxo-3,4-dihydroquinazolin-2-yl)octahydro-1H-indole-1-carboxylate (124 mg, 0.2 mmol, 1 equiv.) with 4N HCl in 1,4-dioxane (1 mL). The reaction was allowed to stir at room temperature for 1 hour. The solvent was removed in vacuo and the crude residue was dissolved in DCM (5 mL). The residual HCl was quenched with sat. aq. NaHCO₃ (5 mL). The layers were shaken and separated, and the aqueous phase was washed with DCM (2×, 5 mL). The combined organics were dried over Na₂SO₄, filtered, and concentrated. The crude reaction mixture was purified via silica gel chromatography (0 to 5% 4:1 (MeOH:sat. aq. NH₄OH) in DCM). 3-(3-fluoro-4-methoxyphenyl)-8-(6-methylpyridin-3-yl)-2-((2S,3aS,7aS)-octahydro-1H-indol-2-yl)-4-oxo-3,4-dihydroquinazoline-6-carbonitrile 25ll was obtained as a white foam (26 mg, 0.05 mmol, 25% yield). 1H NMR (400 MHz, CDCl3) δ 8.66 (d, J=2.2 Hz, 1H), 8.62 (d, J=2.0 Hz, 1H), 7.96 (d, J=1.9 Hz, 1H), 7.79 (dd, J=8.0, 2.3 Hz, 1H), 7.29 (d, J=8.0 Hz, 1H), 7.15-6.94 (m, 3H), 3.97 (d, J=1.1 Hz, 3H), 3.90 (td, J=9.4, 6.0 Hz, 1H), 2.88 (q, J=4.6 Hz, 1H), 2.64 (s, 3H), 1.88 (brs, 1H), 1.81-1.67 (m, 2H), 1.59-1.47 (m, 1H), 1.45-1.24 (m, 5H), 1.15-0.87 (m, 3H). MS (ESI) m/z: 511.0 (M+1)⁺.

### Example 140. Synthesis of 3-(3-fluoro-4-methoxyphenyl)-8-(6-methylpyridin-3-yl)-2-((3aS,6aS)-octahydrocyclopenta[b]pyrrol-2-yl)-4-oxo-3,4-dihydroquinazoline-6-carbonitrile (25 mm)

Prepared as shown in Scheme 8b

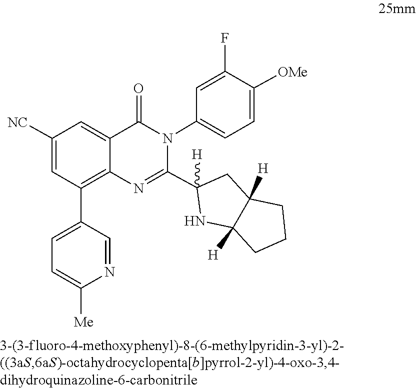 [SMILES: COc1ccc(-n2c(C3C[C@@H]4CCC[C@@H]4N3)nc3c(-c4ccc(C)nc4)cc(C#N)cc3c2=O)cc1F]

### Synthesis of 3-(3-fluoro-4-methoxyphenyl)-8-(6-methylpyridin-3-yl)-2-((3aS,6aS) octahydrocyclopenta[b]pyrrol-2-yl)-4-oxo-3,4-dihydroquinazoline-6-carbonitrile (22p)

A microwave vial with stir bar was charged 2-amino-3-bromo-5-cyanobenzoic acid (275 mg, 1.14 mmol, 1 equiv.), (2S,3aS,6aS)-1-(tert-butoxycarbonyl)octahydrocyclopenta[b]pyrrole-2-carboxylic acid (380 mg, 1.49 mmol, 1.3 equiv.), P(OPh)₃ (0.9 mL, 3.42 mmol, 3 equiv.), and pyridine (2.85 mL). The vial was sealed and heated under microwave irradiation at 130° C. for 40 min. After 40 min, the reaction was cooled and 3-fluoro-4-methoxyaniline (193 mg, 1.37 mmol, 1.2 equiv.) was added in one portion. The vial was sealed and heated under microwave irradiation at 130° C. for 40 min. The reaction was cooled to r.t. and diluted with DCM (20 mL). The reaction mixture was washed with 1N HCl (20 mL), 1N NaOH (20 mL), and brine (20 mL). The organic phase was dried over MgSO₄, filtered, and concentrated. Purified twice by column chromatography (1^{st} column, 0 to 5% EtOAc in DCM, 2^{nd} column 3:1 hexanes:EtOAc). 3-(3-fluoro-4-methoxyphenyl)-8-(6-methylpyridin-3-yl)-2-((3aS,6aS) octahydrocyclopenta[b]pyrrol-2-yl)-4-oxo-3,4-dihydroquinazoline-6-carbonitrile (22p) obtained as a light pink foam (188 mg, 0.32 mmol, 28% yield). (mixture of rotamers) 1H NMR (400 MHz, CDCl₃) δ 8.61-8.40 (m, 1H), 8.29-8.08 (m, 1H), 7.40-7.28 (m, 1H), 7.17-7.05 (m, 1H), 7.01-6.91 (m, 1H), 4.78-4.61 (m, 1H), 4.02-3.92 (m, 3H), 3.92-3.77 (m, 1H), 2.02-1.48 (m, 9H), 1.42 (m, 9H).

### Synthesis of tert-butyl (3aS,6aS)-2-(6-cyano-3-(3-fluoro-4-methoxyphenyl)-8-(6-methylpyridin-3-yl)-4-oxo-3,4-dihydroquinazolin-2-yl)hexahydrocyclopenta[b]pyrrole-1 (2H)-carboxylate

A microwave vial with stir bar was charged with 22p (188 mg, 0.32 mmol, 1 equiv.), boronic acid (48 mg, 0.35 mmol, 1.1 equiv.), Cs₂CO₃ (209 mg, 0.64 mmol, 2 equiv.), and Pd(PPh₃)₄ (37 mg, 0.032 mmol, 0.1 equiv.). A 4:1 mixture of 1,4-dioxane:H₂O was added via syringe (5 mL). The vial was sealed with a septum and the mixture was sparged with N₂ for 15 min. After 15 min, the reaction was heated to 80° C. for 17 h. After 17 h, the reaction was cooled to room temperature and diluted with EtOAc and H₂O (10 mL each). The layers were shaken and separated. The aqueous phase was washed twice with EtOAc (10 mL each). The combined organics were dried over Na₂SO₄, filtered, and concentrated. The crude reaction mixture was purified via silica gel chromatography (3:1 to 1:1 hexanes:EtOAc). Trace palladium was removed by dissolving the purified product in DCM (0.2 M) and adding SiliametS Thiol Metal Scavenger (33 wt %) to the solution. The mixture was heated to 40° C. for 8 h before being filtered through celite. This procedure was repeated two more times. tert-butyl (3aS,6aS)-2-(6-cyano-3-(3-fluoro-4-methoxyphenyl)-8-(6-methylpyridin-3-yl)-4-oxo-3,4-dihydroquinazolin-2-yl)hexahydrocyclopenta[b]pyrrole-1 (2H)-carboxylate was obtained as an off white solid (109 mg, 0.18 mmol, 56% yield). (mixture of rotamers)¹H NMR (400 MHz, CDCl₃) δ 8.64 (m, 2H), 8.14-7.70 (m, 2H), 7.34 (m, 1H), 7.18-6.80 (m, 3H), 4.76-4.33 (m, 1H), 4.24-3.86 (m, 4H), 2.66 (m, 4H), 2.21-1.85 (m, 1H), 1.66 (m, 3H), 1.34 (m, 13H).

### Synthesis of 3-(3-fluoro-4-methoxyphenyl)-8-(6-methylpyridin-3-yl)-2-((3aS,6aS)-octahydrocyclopenta[b]pyrrol-2-yl)-4-oxo-3,4-dihydroquinazoline-6-carbonitrile (25mm)

To a vial with stir bar was added tert-butyl (3aS,6aS)-2-(6-cyano-3-(3-fluoro-4-methoxyphenyl)-8-(6-methylpyridin-3-yl)-4-oxo-3,4-dihydroquinazolin-2-yl)hexahydrocyclopenta[b]pyrrole-1 (2H)-carboxylate (109 mg, 0.2 mmol, 1 equiv.) with 4N HCl in 1,4-dioxane (1 mL). The reaction was allowed to stir at room temperature for 1 hour. The solvent was removed in vacuo and the crude residue was dissolved in DCM (5 mL). The residual HCl was quenched with sat. aq. NaHCO₃ (5 mL). The layers were shaken and separated, and the aqueous phase was washed with DCM (2×, 5 mL). The combined organics were dried over Na₂SO₄, filtered, and concentrated. The crude reaction mixture was purified via silica gel chromatography (0 to 5% (4:1 MeOH:sat. aq. NH₄OH) in DCM). 3-(3-fluoro-4-methoxyphenyl)-8-(6-methylpyridin-3-yl)-2-((3aS,6aS)-octahydrocyclopenta[b]pyrrol-2-yl)-4-oxo-3,4-dihydroquinazoline-6-carbonitrile (25 mm) obtained as a white solid (1.5:1 d.r., 40 mg, 0.081 mmol, 45% yield). ¹H NMR (400 MHz, CDCl₃) δ 8.78-8.55 (m, 2H), 7.98 (d, J=10.3 Hz, 1H), 7.86 (t, J=9.3 Hz, 1H), 7.33-7.25 (m, 1H), 7.06 (ddt, J=37.6, 18.1, 8.8 Hz, 3H), 3.98 (s, 3H), 3.94-3.75 (m, 1H), 3.65-3.42 (m, 1H), 2.66 (s, 3H), 2.51 (d, J=39.2 Hz, 2H), 1.82-1.19 (m, 8H). MS (ESI) m/z: 497.0 (M+1)⁺.

### Example 141. Synthesis of (R)-3-(3-fluoro-4-methoxyphenyl)-8-(6-methylpyridin-3-yl)-2-(2-methylpyrrolidin-2-yl)-4-oxo-3,4-dihydroquinazoline-6-carbonitrile (25nn)

Prepared as shown in Scheme 8b.

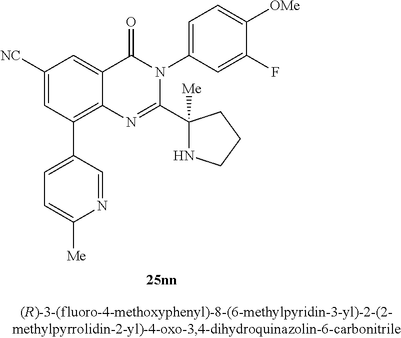 [SMILES: COc1ccc(-n2c([C@@]3(C)CCCN3)nc3c(-c4ccc(C)nc4)cc(C#N)cc3c2=O)cc1F]

### Synthesis of tert-butyl (R)-2-(8-bromo-6-cyano-3-(3-fluoro-4-methoxyphenyl)-4-oxo-3,4-dihydroquinazolin-2-yl)-2-methylpyrrolidine-1-carboxylate (22q)

A microwave vial with stir bar was charged 2-amino-3-bromo-5-cyanobenzoic acid (300 mg, 1.24 mmol, 1 equiv.), (R)-1-(tert-butoxycarbonyl)-2-methylpyrrolidine-2-carboxylic acid (371 mg, 1.62 mmol, 1.3 equiv.), P(OPh)₃ (0.98 mL, 3.72 mmol, 3 equiv.), and pyridine (4.1 mL). The vial was sealed and heated under microwave irradiation at 150° C. for 40 min. After 40 min, the reaction was cooled and 3-fluoro-4-methoxyaniline (201 mg, 1.49 mmol, 1.2 equiv.) was added in one portion. The vial was sealed and heated under microwave irradiation at 150° C. for 40 min. The reaction was cooled to r.t. and diluted with DCM (20 mL). The reaction mixture was washed with 1N HCl (20 mL), 1N NaOH (20 mL), and brine (20 mL). The organic phase was dried over MgSO₄, filtered, and concentrated. Purified twice by column chromatography (1^{st} column, 0:1 to 1:19 EtOAc in DCM, 2^{nd} column, 4:1 to 3:1 hexanes:EtOAc). tert-butyl (R)-2-(8-bromo-6-cyano-3-(3-fluoro-4-methoxyphenyl)-4-oxo-3,4-dihydroquinazolin-2-yl)-2-methylpyrrolidine-1-carboxylate (22q) obtained as ˜50 mg of material of 50% purity. This material was used as is in the next reaction.

### Synthesis of (R)-3-(3-fluoro-4-methoxyphenyl)-8-(6-methylpyridin-3-yl)-2-(2-methylpyrrolidin-2-yl)-4-oxo-3,4-dihydroquinazoline-6-carbonitrile (25nn)

A microwave vial with stir bar was charged with 22q (50 mg, 0.09 mmol, 1 equiv.), boronic acid (14 mg, 0.099 mmol, 1.1 equiv.), Cs₂CO₃ (59 mg, 0.18 mmol, 2 equiv.), and Pd(PPh₃)₄ (10 mg, 0.009 mmol, 0.1 equiv.). A 4:1 mixture of 1,4-dioxane:H₂O was added via syringe (1.3 mL). The vial was sealed with a septum and the mixture was sparged with N₂ for 15 min. After 15 min, the reaction was heated to 80° C. for 17 h. After 17 h, the reaction was cooled to room temperature and diluted with EtOAc and H₂O (2 mL each). The layers were shaken and separated. The aqueous phase was washed twice with EtOAc (2 mL each). The combined organics were dried over Na₂SO₄, filtered, and concentrated. The crude reaction mixture was purified via silica gel chromatography (3:1 to 1:3 hexanes:EtOAc). Trace palladium was removed by dissolving the purified product in DCM (0.2 M) and adding SiliametS Thiol Metal Scavenger (33 wt %) to the solution. The mixture was heated to 40° C. for 8 h before being filtered through celite. This procedure was repeated two more times. Product (15 mg, 0.026 mmol, 1 equiv.) was then added to a vial with stir bar and dissolved in 4N HCl in 1,4-dioxane (1 mL). The reaction was allowed to stir at room temperature for 1 hour. The solvent was removed in vacuo and the crude residue was dissolved in DCM (5 mL). The residual HCl was quenched with sat. aq. NaHCO₃ (5 mL). The layers were shaken and separated, and the aqueous phase was washed with DCM (2×, 5 mL). The combined organics were dried over Na₂SO₄, filtered, and concentrated. The crude reaction mixture was purified via silica gel chromatography (2% to 5% (4:1 MeOH:sat. aq. NH₄OH) in DCM). (R)-3-(3-fluoro-4-methoxyphenyl)-8-(6-methylpyridin-3-yl)-2-(2-methylpyrrolidin-2-yl)-4-oxo-3,4-dihydroquinazoline-6-carbonitrile (25nn) obtained as a light yellow film (9 mg, 0.019 mmol, 22% over 2 steps)¹H NMR (400 MHz, CDCl₃) δ 8.74 (s, 1H), 8.56 (d, J=1.8 Hz, 1H), 8.01-7.96 (m, 1H), 7.89 (dd, J=8.0, 2.4 Hz, 1H), 7.31 (d, J=8.0 Hz, 1H), 7.17-6.97 (m, 3H), 3.98 (s, 3H), 2.98-2.85 (m, 1H), 2.77 (ddt, J=12.4, 9.9, 7.3 Hz, 1H), 2.66 (s, 3H), 2.23 (ddt, J=35.1, 12.7, 8.1 Hz, 1H), 1.72 (ddtd, J=24.7, 12.6, 7.9, 4.4 Hz, 2H), 1.44 (ddd, J=12.7, 7.7, 4.9 Hz, 1H), 1.32 (s, 3H). MS (ESI) m/z: 470.0 (M+1)⁺.

### Example 142. Synthesis of (S)-3-(3-fluoro-4-methoxyphenyl)-8-(6-methylpyridin-3-yl)-2-(2-methylpyrrolidin-2-yl)-4-oxo-3,4-dihydroquinazoline-6-carbonitrile (25oo)

Prepared as shown in Scheme 8b.

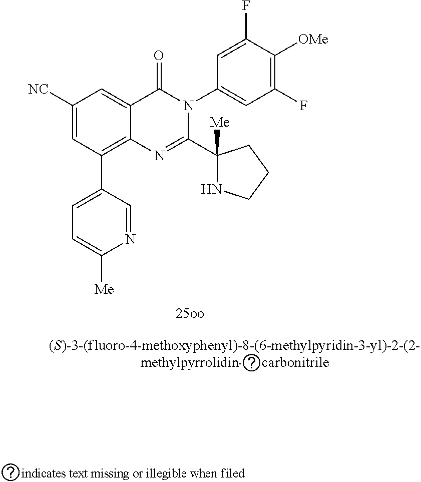 [SMILES: COc1c(F)cc(-n2c([C@]3(C)CCCN3)nc3c(-c4ccc(C)nc4)cc(C#N)cc3c2=O)cc1F]

### Synthesis of tert-butyl (R)-2-(8-bromo-6-cyano-3-(3-fluoro-4-methoxyphenyl)-4-oxo-3,4-dihydroquinazolin-2-yl)-2-methylpyrrolidine-1-carboxylate 22r

A microwave vial with stir bar was charged 2-amino-3-bromo-5-cyanobenzoic acid (300 mg, 1.24 mmol, 1 equiv.), (S)-1-(tert-butoxycarbonyl)-2-methylpyrrolidine-2-carboxylic acid (371 mg, 1.62 mmol, 1.3 equiv.), P(OPh)₃ (0.98 mL, 3.72 mmol, 3 equiv.), and pyridine (4.1 mL). The vial was sealed and heated under microwave irradiation at 150° C. for 40 min. After 40 min, the reaction was cooled and 3-fluoro-4-methoxyaniline (201 mg, 1.49 mmol, 1.2 equiv.) was added in one portion. The vial was sealed and heated under microwave irradiation at 150° C. for 40 min. The reaction was cooled to r.t. and diluted with DCM (20 mL). The reaction mixture was washed with 1N HCl (20 mL), 1N NaOH (20 mL), and brine (20 mL). The organic phase was dried over MgSO₄, filtered, and concentrated. Purified twice by column chromatography (1^{st} column, 0:1 to 1:19 EtOAc in DCM, 2^{nd} column, 4:1 to 3:1 hexanes:EtOAc). tert-butyl (R)-2-(8-bromo-6-cyano-3-(3-fluoro-4-methoxyphenyl)-4-oxo-3,4-dihydroquinazolin-2-yl)-2-methylpyrrolidine-1-carboxylate (22r) obtained as 62 mg of material of 50% purity. This material was used as is in the next reaction.

### Synthesis of (S)-3-(3-fluoro-4-methoxyphenyl)-8-(6-methylpyridin-3-yl)-2-(2-methylpyrrolidin-2-yl)-4-oxo-3,4-dihydroquinazoline-6-carbonitrile (25oo)

A microwave vial with stir bar was charged with 22r (62 mg, 0.11 mmol, 1 equiv.), boronic acid (17 mg, 0.12 mmol, 1.1 equiv.), Cs₂CO₃ (72 mg, 0.22 mmol, 2 equiv.), and Pd(PPh₃)₄ (13 mg, 0.011 mmol, 0.1 equiv.). A 4:1 mixture of 1,4-dioxane:H₂O was added via syringe (1.6 mL). The vial was sealed with a septum and the mixture was sparged with N₂ for 15 min. After 15 min, the reaction was heated to 80° C. for 17 h. After 17 h, the reaction was cooled to room temperature and diluted with EtOAc and H₂O (2 mL each). The layers were shaken and separated. The aqueous phase was washed twice with EtOAc (2 mL each). The combined organics were dried over Na₂SO₄, filtered, and concentrated. The crude reaction mixture was purified via silica gel chromatography (3:1 to 1:3 hexanes:EtOAc). Trace palladium was removed by dissolving the purified product in DCM (0.2 M) and adding SiliametS Thiol Metal Scavenger (33 wt %) to the solution. The mixture was heated to 40° C. for 8 h before being filtered through celite. This procedure was repeated two more times. The product (9 mg, 0.016 mmol, 1 equiv.) was added to a vial with stir bar and dissolved in 4N HCl in 1,4-dioxane (1 mL). The reaction was allowed to stir at room temperature for 1 hour. The solvent was removed in vacuo and the crude residue was dissolved in DCM (5 mL). The residual HCl was quenched with sat. aq. NaHCO₃ (5 mL). The layers were shaken and separated, and the aqueous phase was washed with DCM (2×, 5 mL). The combined organics were dried over Na₂SO₄, filtered, and concentrated. The crude reaction mixture was purified via silica gel chromatography (0% to 10% MeOH in DCM). (S)-3-(3-fluoro-4-methoxyphenyl)-8-(6-methylpyridin-3-yl)-2-(2-methylpyrrolidin-2-yl)-4-oxo-3,4-dihydroquinazoline-6-carbonitrile (2500) obtained as a light yellow film (6.5 mg, 0.014 mmol, 13% over 2 steps)¹H NMR (400 MHz, CDCl₃) δ 8.74 (s, 1H), 8.56 (d, J=1.8 Hz, 1H), 8.01-7.96 (m, 1H), 7.89 (dd, J=8.0, 2.4 Hz, 1H), 7.31 (d, J=8.0 Hz, 1H), 7.17-6.97 (m, 3H), 3.98 (s, 3H), 2.98-2.85 (m, 1H), 2.77 (ddt, J=12.4, 9.9, 7.3 Hz, 1H), 2.66 (s, 3H), 2.23 (ddt, J=35.1, 12.7, 8.1 Hz, 1H), 1.72 (ddtd, J=24.7, 12.6, 7.9, 4.4 Hz, 2H), 1.44 (ddd, J=12.7, 7.7, 4.9 Hz, 1H), 1.32 (s, 3H). MS (ESI) m/z: 470.0 (M+1)⁺.

### Example 143. Synthesis of 2-(7-azabicyclo[2.2.1]heptan-2-yl)-3-(3-fluoro-4-methoxyphenyl)-8-(6-methylpyridin-3-yl)-4-oxo-3,4-dihydroquinazoline-6-carbonitrile (25pp)

Prepared as shown in Scheme 8b.

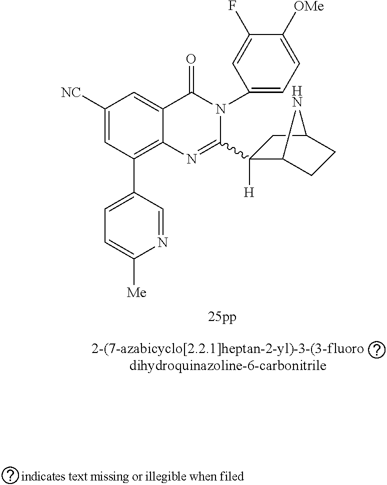 [SMILES: COc1ccc(-n2c(C3CC4CCC3N4)nc3c(-c4ccc(C)nc4)cc(C#N)cc3c2=O)cc1F]

### Synthesis of rac-tert-butyl 2-(8-bromo-6-cyano-3-(3-fluoro-4-methoxyphenyl)-4-oxo-3,4-dihydroquinazolin-2-yl)-7-azabicyclo[2.2.1]heptane-7-carboxylate (22s)

A microwave vial with stir bar was charged 2-amino-3-bromo-5-cyanobenzoic acid (300 mg, 1.24 mmol, 1 equiv.), rac-7-(tert-butoxycarbonyl)-7-azabicyclo[2.2.1]heptane-2-carboxylic acid (390 mg, 1.62 mmol, 1.3 equiv.), P(OPh)₃ (0.98 mL, 3.72 mmol, 3 equiv.), and pyridine (4.1 mL). The vial was sealed and heated under microwave irradiation at 130° C. for 40 min. After 40 min, the reaction was cooled and 3-fluoro-4-methoxyaniline (201 mg, 1.49 mmol, 1.2 equiv.) was added in one portion. The vial was sealed and heated under microwave irradiation at 110° C. for 40 min. The reaction was cooled to r.t. and diluted with DCM (20 mL). The reaction mixture was washed with 1N HCl (20 mL), 1N NaOH (20 mL), and brine (20 mL). The organic phase was dried over MgSO₄, filtered, and concentrated. Purified twice by column chromatography (1^{st} column, 0:1 to 1:19 EtOAc in DCM, 2^{nd} column, 3:1 to 1:1 hexanes:EtOAc). rac-tert-butyl 2-(8-bromo-6-cyano-3-(3-fluoro-4-methoxyphenyl)-4-oxo-3,4-dihydroquinazolin-2-yl)-7-azabicyclo[2.2.1]heptane-7-carboxylate (22s) obtained as a yellow oil (46 mg, 0.081 mmol, 7% yield). (Mixture of rotamers)¹H NMR (400 MHz, CDCl₃) δ 8.50 (s, 1H), 8.20 (s, 1H), 7.37-7.28 (m, 1H), 7.19-7.09 (m, 1H), 7.00-6.90 (m, 1H), 4.48-4.32 (m, 2H), 3.99 (d, J=3.3 Hz, 3H), 2.86 (p, J=6.0, 5.5 Hz, 2H), 1.83-1.52 (m, 5H), 1.39-1.27 (m, 9H).

### Synthesis of 2-(7-azabicyclo[2.2.1]heptan-2-yl)-3-(3-fluoro-4-methoxyphenyl)-8-(6-methylpyridin-3-yl)-4-oxo-3,4-dihydroquinazoline-6-carbonitrile (25pp)

A microwave vial with stir bar was charged with 22s (46 mg, 0.081 mmol, 1 equiv.), boronic acid (12 mg, 0.089 mmol, 1.1 equiv.), Cs₂CO₃ (53 mg, 0.16 mmol, 2 equiv.), and Pd(PPh₃)₄ (9 mg, 0.0081 mmol, 0.1 equiv.). A 4:1 mixture of 1,4-dioxane:H₂O was added via syringe (1.2 mL). The vial was sealed with a septum and the mixture was sparged with N₂ for 15 min. After 15 min, the reaction was heated to 80° C. for 17 h. After 17 h, the reaction was cooled to room temperature and diluted with EtOAc and H₂O (10 mL each). The layers were shaken and separated. The aqueous phase was washed twice with EtOAc (10 mL each). The combined organics were dried over Na₂SO₄, filtered, and concentrated. The crude reaction mixture was purified via silica gel chromatography (3:1 to 1:9 hexanes:EtOAc). Trace palladium was removed by dissolving the purified product in DCM (0.2 M) and adding SiliametS Thiol Metal Scavenger (33 wt %) to the solution. The mixture was heated to 40° C. for 8 h before being filtered through celite. This procedure was repeated two more times. tert-butyl 2-(6-cyano-3-(3-fluoro-4-methoxyphenyl)-8-(6-methylpyridin-3-yl)-4-oxo-3,4-dihydroquinazolin-2-yl)-7-azabicyclo[2.2.1]heptane-7-carboxylate obtained as a mixture of desired product and triphenylphosphine oxide (TPPO) which was used without further purification. This product was added to a vial with stir bar and dissolved in 4N HCl in 1,4-dioxane (1 mL). The reaction was allowed to stir at room temperature for 1 hour. The solvent was removed in vacuo and the crude residue was dissolved in DCM (5 mL). The residual HCl was quenched with sat. aq. NaHCO₃ (5 mL). The layers were shaken and separated, and the aqueous phase was washed with DCM (2×, 5 mL). The combined organics were dried over Na₂SO₄, filtered, and concentrated. The crude reaction mixture was purified via silica gel chromatography (0 to 5% (4:1 MeOH:sat. aq. NH₄OH) in DCM). 2-(7-azabicyclo[2.2.1]heptan-2-yl)-3-(3-fluoro-4-methoxyphenyl)-8-(6-methylpyridin-3-yl)-4-oxo-3,4-dihydroquinazoline-6-carbonitrile 25pp (18 mg, 0.037 mmol, 46% yield over 2 steps) was obtained as an off-white solid. ¹H NMR (400 MHz, CDCl₃) δ 8.70 (d, J=2.0 Hz, 1H), 8.58 (d, J=1.4 Hz, 1H), 7.96 (d, J=1.9 Hz, 1H), 7.85 (dt, J=8.0, 2.7 Hz, 1H), 7.33 (d, J=8.1 Hz, 1H), 7.15 (q, J=8.5 Hz, 1H), 7.09-6.90 (m, 2H), 3.99 (s, 3H), 3.80-3.58 (m, 2H), 2.99 (brs, 1H), 2.71-2.56 (m, 4H), 1.93-1.76 (m, 1H), 1.64 (m, 3H), 0.94 (m, 2H). MS (ESI) m/z: 483.0 (M+1)⁺.

### Example 144. Synthesis of (R)-3-(3-fluoro-4-(2-methoxyethoxy)phenyl)-8-(6-methylpyridin-3-yl)-4-oxo-2-(pyrrolidin-2-yl)-3,4-dihydroquinazoline-6-carbonitrile (25qq)

Prepared as shown in Scheme 8b.

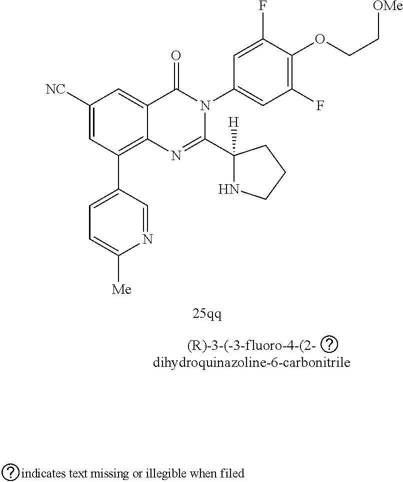 [SMILES: COCCOc1c(F)cc(-n2c([C@H]3CCCN3)nc3c(-c4ccc(C)nc4)cc(C#N)cc3c2=O)cc1F]

The BOC-protected penultimate intermediate (218 mg, 0.364 mmol), was deprotected in CH₂Cl₂ (6.0 mL) and trifluoroacetic acid (835 μL, 10.9 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash (MeOH:CH₂Cl₂=5:100) yielded 25qq (90.2 mg, 50%) as a white solid (LC/MS purity=99.6%). ¹H NMR (400 MHz, CDCl₃): δ=8.74 (d, J=2.0 Hz, 1H), 8.59 (d, J=1.6 Hz, 1H), 7.98 (d, J=1.8 Hz, 1H), 7.85 (dd, J=8.0, 2.2 Hz, 1H), 7.30 (d, J=8.0 Hz, 1H), 7.16 (t, J=8.8 Hz, 1H), 7.11-6.95 (m, 2H), 4.32-4.23 (m, 2H), 3.92-3.87 (m, 1H), 3.86-3.78 (m, 2H), 3.48 (s, 3H), 3.15-2.96 (m, 1H), 2.78-2.70 (m, 1H), 2.67 (s, 3H), 2.33 (brs, 1H), 1.83-1.65 (m, 4H).

### Example 145. Synthesis of 3-(4-(Dimethylamino)-3-fluorophenyl)-8-(6-methylpyridin-3-yl)-4-oxo-2-(pyrrolidin-2-yl)-3,4-dihydroquinazoline-6-carbonitrile (25rr)

Prepared as shown in Scheme 8b.

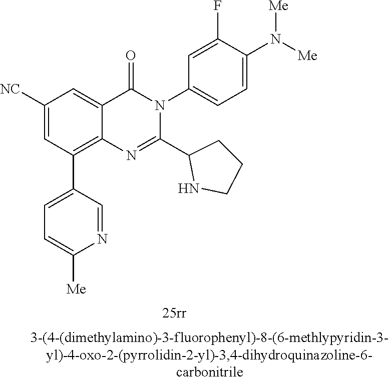 [SMILES: Cc1ccc(-c2cc(C#N)cc3c(=O)n(-c4ccc(N(C)C)c(F)c4)c(C4CCCN4)nc23)cn1]

The BOC-protected penultimate intermediate (131 mg, 0.230 mmol) was deprotected in CH₂Cl₂ (4.0 mL) and trifluoroacetic acid (528 μL, 6.90 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash (MeOH:CH₂Cl₂=5:100) yielded 25rr (29.3 mg, 27%) as a white solid (LC/MS purity=98.0%). ¹H NMR (400 MHz, CDCl₃): δ=8.74 (d, J=2.0 Hz, 1H), 8.60 (d, J=1.9 Hz, 1H), 7.97 (d, J=1.9 Hz, 1H), 7.85 (dd, J=8.0, 2.3 Hz, 1H), 7.30 (d, J=8.0 Hz, 1H), 7.01-6.83 (m, 3H), 3.93 (q, J=7.0, 6.5 Hz, 1H), 3.15-3.05 (m, 1H), 2.97 (s, 6H), 2.87-2.70 (m, 1H), 2.67 (s, 3H), 2.38 (brs, 1H), 1.89-1.64 (m, 4H).

### Example 146. Synthesis of 3-(3-Fluoro-4-morpholinophenyl)-8-(6-methylpyridin-3-yl)-4-oxo-2-(pyrrolidin-2-yl)-3,4-dihydroquinazoline-6-carbonitrile (25ss)

Prepared as shown in Scheme 8b.

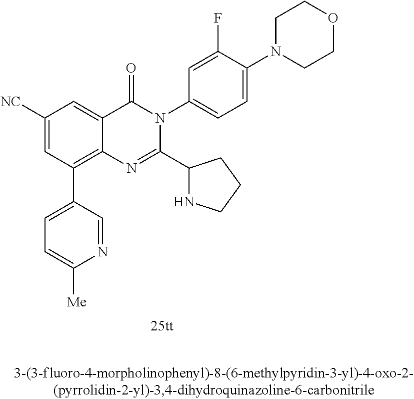 [SMILES: Cc1ccc(-c2cc(C#N)cc3c(=O)n(-c4ccc(N5CCOCC5)c(F)c4)c(C4CCCN4)nc23)cn1]

The BOC-protected penultimate intermediate (276 mg, 0.452 mmol) was deprotected in CH₂Cl₂ (8.0 mL) and trifluoroacetic acid (1.04 mL, 13.6 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash (MeOH:CH₂Cl₂=5:100) yielded 25ss (144 mg, 62%) as a white solid (LC/MS purity=98.8%). ¹H NMR (400 MHz, CDCl₃): δ=8.74 (d, J=1.9 Hz, 1H), 8.60 (d, J=1.9 Hz, 1H), 7.98 (d, J=1.4 Hz, 1H), 7.85 (dd, J=8.0, 2.3 Hz, 1H), 7.30 (d, J=8.0 Hz, 1H), 7.13-6.88 (m, 3H), 3.93-3.86 (m, 5H), 3.27-3.23 (m, 2H), 3.21-3.12 (m, 2H), 3.11-3.05 (m, 1H), 2.78-2.72 (m, 1H), 2.67 (s, 3H), 2.40 (brs, 1H), 1.83-1.67 (m, 4H).

### Example 147. Synthesis of 3-(3-Fluoro-4-methoxyphenyl)-8-(4-methoxyphenyl)-4-oxo-2-(pyrrolidin-2-yl)-3,4-dihydroquinazoline-6-carbonitrile (25tt)

Prepared as shown in Scheme 8b.

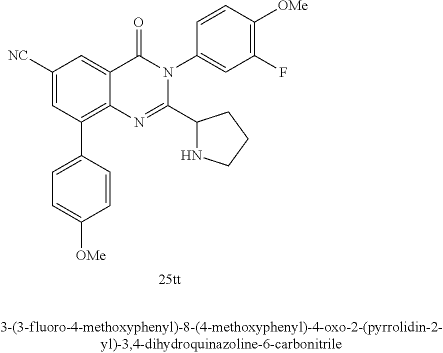 [SMILES: COc1ccc(-c2cc(C#N)cc3c(=O)n(-c4ccc(OC)c(F)c4)c(C4CCCN4)nc23)cc1]

The BOC-protected penultimate intermediate (100 mg, 0.175 mmol) was deprotected in CH₂Cl₂ (3.0 mL) and trifluoroacetic acid (268 μL, 3.50 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash (MeOH:CH₂Cl₂=5:100) yielded 25tt (92.6 mg, 99%) as a pale yellow solid (LC/MS purity=99.6%). ¹H NMR (400 MHz, CDCl₃): δ=8.54 (d, J=1.8 Hz, 1H), 7.97 (d, J=2.2 Hz, 1H), 7.57 (d, J=8.6 Hz, 2H), 7.17-7.09 (m, 1H), 7.09-6.97 (m, 4H), 3.98 (s, 3H), 3.95-3.87 (m, 1H), 3.90 (s, 3H), 3.11-3.06 (m, 1H), 2.93 (brs, 1H), 2.79-2.69 (m, 1H), 1.86-1.59 (m, 4H).

### Example 148. Synthesis of 3-(3-Fluoro-4-methoxyphenyl)-8-(3-methoxyphenyl)-4-oxo-2-(pyrrolidin-2-yl)-3,4-dihydroquinazoline-6-carbonitrile (25uu)

Prepared as shown in Scheme 8b.

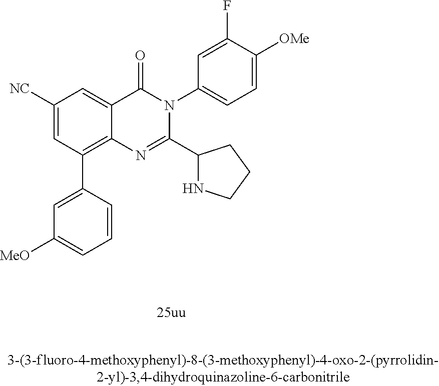 [SMILES: COc1cccc(-c2cc(C#N)cc3c(=O)n(-c4ccc(OC)c(F)c4)c(C4CCCN4)nc23)c1]

The BOC-protected penultimate intermediate (166 mg, 0.291 mmol) was deprotected in CH₂Cl₂ (5.0 mL) and trifluoroacetic acid (668 μL, 8.73 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash (MeOH:CH₂Cl₂=5:100) yielded 25uu (109 mg, 80%) as a white solid (LC/MS purity=98.6%). ¹H NMR (400 MHz, CDCl₃): δ=8.57 (d, J=2.0 Hz, 1H), 8.00 (d, J=2.2 Hz, 1H), 7.40 (t, J=7.9 Hz, 1H), 7.19-7.14 (m, 2H), 7.14-7.09 (m, 1H), 7.08-7.05 (m, 1H), 7.03-7.01 (m, 1H), 7.01-6.97 (m, 1H), 3.98 (s, 3H), 3.95-3.88 (m, 1H), 3.87 (s, 3H), 3.61 (brs, 1H), 3.12-2.95 (m, 1H), 2.89-2.63 (m, 1H), 1.86-1.62 (m, 4H).

### Example 149. Synthesis of 3,8-Bis(3-fluoro-4-methoxyphenyl)-4-oxo-2-(pyrrolidin-2-yl)-3,4-dihydroquinazoline-6-carbonitrile (25vv)

Prepared as shown in Scheme 8b.

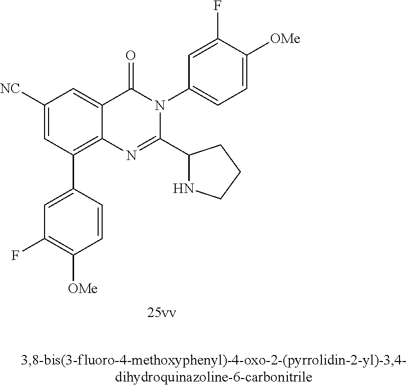 [SMILES: COc1ccc(-c2cc(C#N)cc3c(=O)n(-c4ccc(OC)c(F)c4)c(C4CCCN4)nc23)cc1F]

The BOC-protected penultimate intermediate (153 mg, 0.260 mmol) was deprotected in CH₂Cl₂ (4.3 mL) and trifluoroacetic acid (597 μL, 7.80 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash (MeOH:CH₂Cl₂=5:100) yielded 25vv (71.4 mg, 56%) as a white solid (LC/MS purity=99.6%). ¹H NMR (400 MHz, CDCl₃): δ=8.55 (d, J=2.1 Hz, 1H), 7.97-7.96 (m, 1H), 7.47 (d, J=12.4 Hz, 1H), 7.32 (d, J=8.4 Hz, 1H), 7.15-7.11 (m, 1H), 7.10-7.08 (m, 1H), 7.08-7.05 (m, 1H), 7.04-6.98 (m, 1H), 3.98 (s, 3H), 3.98 (s, 3H), 3.96-3.88 (m, 1H), 3.17-3.05 (m, 1H), 2.85-2.73 (m, 1H), 2.59 (brs, 1H), 1.87-1.67 (m, 4H).

### Example 150. Synthesis of 3-(3-Fluoro-4-methoxyphenyl)-8-(1-methyl-1H-indol-3-yl)-4-oxo-2-(pyrrolidin-2-yl)-3,4-dihydroquinazoline-6-carbonitrile (25ww)

Prepared as shown in Scheme 8b.

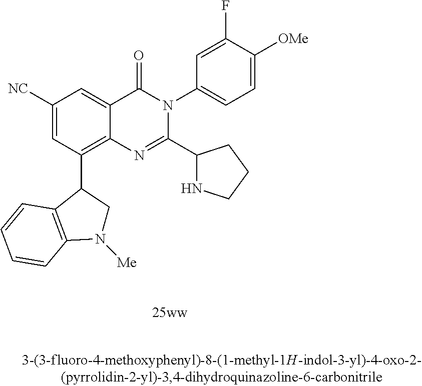 [SMILES: COc1ccc(-n2c(C3CCCN3)nc3c(C4CN(C)c5ccccc54)cc(C#N)cc3c2=O)cc1F]

The BOC-protected penultimate intermediate (140 mg, 0.236 mmol) was deprotected in CH₂Cl₂ (4.0 mL) and trifluoroacetic acid (542 μL, 7.08 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 25ww (83.3 mg, 72%) as a pale yellow (LC/MS purity=99.1%). ¹H NMR (400 MHz, CDCl₃): δ=8.47 (d, J=1.4 Hz, 1H), 8.22 (d, J=1.9 Hz, 1H), 7.77 (d, J=7.9 Hz, 1H), 7.61 (d, J=2.4 Hz, 1H), 7.43 (d, J=8.2 Hz, 1H), 7.33 (t, J=7.2 Hz, 1H), 7.23 (t, J=7.3 Hz, 1H), 7.16-6.98 (m, 3H), 3.98 (s, 3H), 3.92 (s, 3H), 3.91-3.84 (m, 1H), 3.05-2.94 (m, 1H), 2.59 (brs, 1H), 2.74-2.62 (m, 1H), 1.86-1.57 (m, 4H).

### Example 151. Synthesis of 3-(3-Fluoro-4-methoxyphenyl)-4-oxo-2-(pyrrolidin-2-yl)-8-(4-(trifluoromethoxy)phenyl)-3,4-dihydroquinazoline-6-carbonitrile (25xx)

Prepared as shown in Scheme 8b.

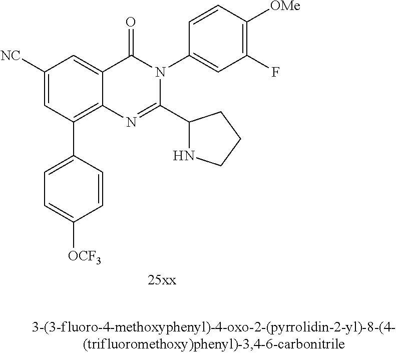 [SMILES: COc1ccc(-n2c(C3CCCN3)nc3c(-c4ccc(OC(F)(F)F)cc4)cc(C#N)cc3c2=O)cc1F]

The BOC-protected penultimate intermediate (52.8 mg, 0.0845 mmol) was deprotected in CH₂Cl₂ (1.4 mL) and trifluoroacetic acid (195 μL, 2.54 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 25xx (36.2 mg, 82%) as a pale yellow solid (LC/MS purity=97.6%).

¹H NMR (400 MHz, CDCl₃): δ=8.58 (d, J=1.8 Hz, 1H), 7.97 (d, J=1.8 Hz, 1H), 7.64 (d, J=8.7 Hz, 2H), 7.35 (d, J=8.3 Hz, 2H), 7.15-7.10 (m, 1H), 7.09-7.00 (m, 2H), 3.98 (s, 3H), 3.93-3.83 (m, 1H), 3.08-2.96 (m, 1H), 2.78-2.69 (m, 1H), 2.68 (brs, 1H), 1.88-1.63 (m, 4H).

### Example 152. Synthesis of 8-(3,4-Dimethoxyphenyl)-3-(3-fluoro-4-methoxyphenyl)-4-oxo-2-(pyrrolidin-2-yl)-3,4-dihydroquinazoline-6-carbonitrile (25yy)

Prepared as shown in Scheme 8b.

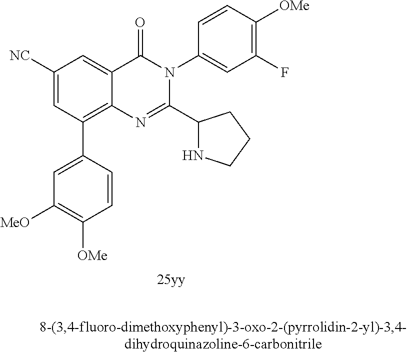 [SMILES: COc1ccc(-n2c(C3CCCN3)nc3c(-c4ccc(OC)c(OC)c4)cc(C#N)cc3c2=O)cc1F]

The BOC-protected penultimate intermediate (67.6 mg, 0.113 mmol) was deprotected in CH₂Cl₂ (1.9 mL) and trifluoroacetic acid (260 μL, 3.39 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 25yy (19.4 mg, 34%) as a pale yellow solid (LC/MS purity=99.1%).

¹H NMR (400 MHz, CDCl₃): δ=8.51 (s, 1H), 7.97 (s, 1H), 7.23 (s, 1H), 7.16 (d, J=7.9 Hz, 1H), 7.13 (s, 1H), 7.10 (d, J=10.5 Hz, 1H), 7.05 (d, J=10.5 Hz, 1H), 7.00 (d, J=7.9 Hz, 1H), 3.98 (s, 6H), 3.95 (s, 3H), 3.91-3.81 (m, 1H), 3.13-3.04 (m, 1H), 2.81 (brs, 1H), 2.77-2.65 (m, 1H), 1.88-1.54 (m, 4H).

### Example 153. Synthesis of 3-(3-Fluoro-4-methoxyphenyl)-8-(4-morpholinophenyl)-4-oxo-2-(pyrrolidin-2-yl)-3,4-dihydroquinazoline-6-carbonitrile (25zz)

Prepared as shown in Scheme 8b.

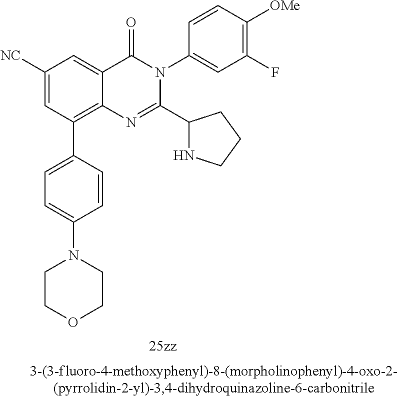 [SMILES: COc1ccc(-n2c(C3CCCN3)nc3c(-c4ccc(N5CCOCC5)cc4)cc(C#N)cc3c2=O)cc1F]

The BOC-protected penultimate intermediate (107 mg, 0.171 mmol) was deprotected in CH₂Cl₂ (5.0 mL) and trifluoroacetic acid (393 μL, 5.13 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 25zz (24.0 mg, 28%) as a yellow solid (LC/MS purity=99.7%).

¹H NMR (400 MHz, CDCl₃): δ=8.51 (d, J=2.0 Hz, 1H), 7.97 (d, J=2.0 Hz, 1H), 7.59 (d, J=8.8 Hz, 2H), 7.14-7.10 (m, 1H), 7.09-7.06 (m, 1H), 7.05-7.04 (m, 1H), 7.01 (d, J=8.8 Hz, 2H), 3.98 (s, 3H), 3.92-3.90 (m, 4H), 3.89-3.85 (m, 1H), 3.29-3.27 (m, 4H), 3.13-3.09 (m, 1H), 2.77-2.73 (m, 1H), 1.77-1.68 (m, 4H).

### Example 154. Synthesis of 3-(3-Fluoro-4-methoxyphenyl)-8-(4-(2-methoxyethoxy)phenyl)-4-oxo-2-(pyrrolidin-2-yl)-3,4-dihydroquinazoline-6-carbonitrile (25aaa)

Prepared as shown in Scheme 8b.

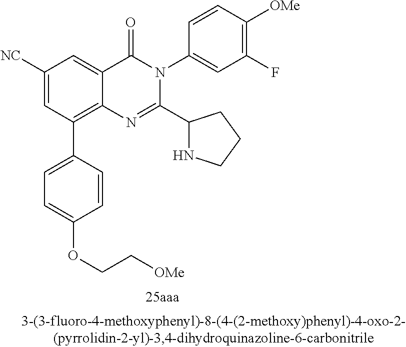 [SMILES: COCCOc1ccc(-c2cc(C#N)cc3c(=O)n(-c4ccc(OC)c(F)c4)c(C4CCCN4)nc23)cc1]

The BOC-protected penultimate intermediate (80.8 mg, 0.131 mmol) was deprotected in CH₂Cl₂ (5.0 mL) and trifluoroacetic acid (301 μL, 3.93 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 25aaa (45.5 mg, 67%) as a white solid (LC/MS purity=97.7%). ¹H NMR (400 MHz, CDCl₃): δ=8.51 (s, 1H), 7.95 (s, 1H), 7.56 (d, J=8.4 Hz, 2H), 7.15-7.10 (m, 1H), 7.09-7.06 (m, 1H), 7.05-7.04 (m, 2H), 7.03-6.97 (m, 1H), 4.22-4.21 (m, 2H), 3.97 (s, 3H), 3.89 (dd, J=5.6 Hz, 5.3 Hz, 1H), 3.82-3.80 (m, 2H), 3.49 (s, 3H), 3.10-3.07 (m, 1H), 2.79 (brs, 1H), 2.76-2.71 (m, 1H), 1.77-1.67 (m, 4H).

### Example 155. Synthesis of 3-(3,5-Difluoro-4-methoxyphenyl)-8-(2-methylpyridin-4-yl)-4-oxo-2-(pyrrolidin-2-yl)-3,4-dihydroquinazoline-6-carbonitrile (25bbb)

Prepared as shown in Scheme 8b.

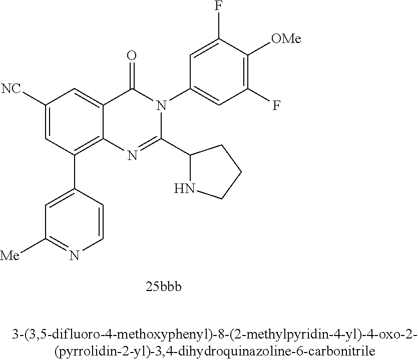 [SMILES: COc1c(F)cc(-n2c(C3CCCN3)nc3c(-c4ccnc(C)c4)cc(C#N)cc3c2=O)cc1F]

The BOC-protected penultimate intermediate (192 mg, 0.325 mmol) was deprotected in CH₂Cl₂ (5.4 mL) and trifluoroacetic acid (773 μL, 10.1 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 25bbb (121 mg, 76%) as a white solid (LC/MS purity=99.9%). ¹H NMR (400 MHz, CDCl₃): δ=8.63 (d, J=5.2 Hz, 1H), 8.61 (d, J=1.7 Hz, 1H), 8.01 (s, 1H), 7.41 (s, 1H), 7.34 (d, J=5.1 Hz, 1H), 6.94 (d, J=10.3 Hz, 1H), 6.87 (d, J=10.3 Hz, 1H), 4.12 (s, 3H), 3.89 (t, J=6.7 Hz, 1H), 3.08-3.03 (m, 1H), 2.82-2.75 (m, 1H), 2.67 (s, 3H), 2.44 (brs, 1H), 1.86-1.72 (m, 4H).

### Example 156. Synthesis of 3-(3,5-Difluoro-4-methoxyphenyl)-2-((4S)-4-fluoropyrrolidin-2-yl)-8-(2-methylpyridin-4-yl)-4-oxo-3,4-dihydroquinazoline-6-carbonitrile (25ccc)

Prepared as shown in Scheme 8b.

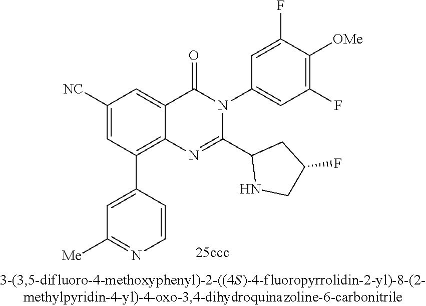 [SMILES: COc1c(F)cc(-n2c(C3C[C@H](F)CN3)nc3c(-c4ccnc(C)c4)cc(C#N)cc3c2=O)cc1F]

The BOC-protected penultimate intermediate (47.4 mg, 0.0801 mmol) was deprotected in CH₂Cl₂ (1.4 mL) and trifluoroacetic acid (84 μL, 2.40 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 25ccc (31.9 mg, 81%) as a white solid (LC/MS purity=99.9%). ¹H NMR (400 MHz, CDCl₃): δ=8.72-8.50 (m, 2H), 8.00 (s, 1H), 7.36 (s, 1H), 7.30 (d, J=3.9 Hz, 1H), 7.05 (d, J=10.3 Hz, 1H), 6.86 (d, J=10.0 Hz, 1H), 5.18 (d, J=54.2 Hz, 1H), 4.20 (t, J=7.2 Hz, 1H), 4.13 (s, 3H), 3.18-2.84 (m, 2H), 2.66 (s, 3H), 2.36 (brs, 1H), 2.46-2.22 (m, 1H), 2.20-2.04 (m, 1H).

### Example 157. Synthesis of N-(4-(6-Cyano-3-(3-fluoro-4-methoxyphenyl)-4-oxo-2-(pyrrolidin-2-yl)-3,4-dihydroquinazolin-8-yl)phenyl)methanesulfonamide (25ddd)

Prepared as shown in Scheme 8b.

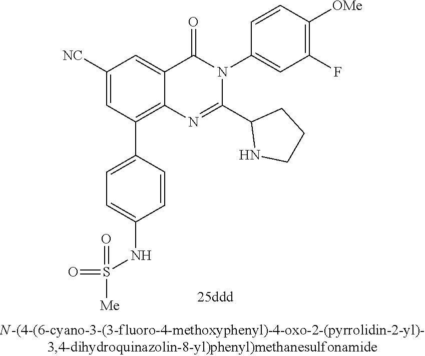 [SMILES: COc1ccc(-n2c(C3CCCN3)nc3c(-c4ccc(NS(C)(=O)=O)cc4)cc(C#N)cc3c2=O)cc1F]

The BOC-protected penultimate intermediate (41.3 mg, 0.0652 mmol) was deprotected in CH₂Cl₂ (0.6 mL) and trifluoroacetic acid (150 μL, 1.96 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 25ddd (22.8 mg, 65%) as a white solid (LC/MS purity=97.6%). ¹H NMR (400 MHz, CDCl₃): δ=8.48 (d, J=1.7 Hz, 1H), 8.26 (d, J=1.7 Hz, 1H), 7.72 (d, J=8.7 Hz, 2H), 7.49 (d, J=11.9 Hz, 1H), 7.38-7.29 (m, 4H), 3.93 (s, 3H), 3.78 (dd, J=7.6, 7.5 Hz, 1H), 3.06 (s, 3H), 2.92-2.86 (m, 1H), 2.65-2.59 (m, 1H), 1.97-1.85 (m, 1H), 1.62-1.60 (m, 1H), 1.58-1.50 (m, 2H).

### Example 158. Synthesis of 3-(3-Fluoro-4-methoxyphenyl)-8-(4-hydroxyphenyl)-4-oxo-2-(pyrrolidin-2-yl)-3,4-dihydroquinazoline-6-carbonitrile (25eee)

Prepared as shown in Scheme 8b.

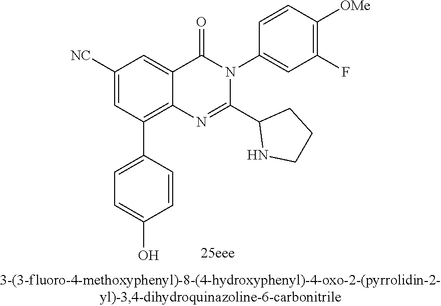 [SMILES: COc1ccc(-n2c(C3CCCN3)nc3c(-c4ccc(O)cc4)cc(C#N)cc3c2=O)cc1F]

The BOC-protected penultimate intermediate (42.7 mg, 0.0767 mmol) was deprotected in CH₂Cl₂ (0.8 mL) and trifluoroacetic acid (176 μL, 2.30 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 25eee (23.9 mg, 68%) as a yellow solid (LC/MS purity=99.7%). ¹H NMR (400 MHz, CDCl₃): δ=9.73 (brs, 1H), 8.42-8.41 (m, 1H), 8.19-8.17 (m, 1H), 7.59 (d, J=8.6 Hz, 2H), 7.49 (dt, J=11.7, 2.3 Hz, 1H), 7.38-7.32 (m, 1H), 7.31-7.28 (m, 1H), 6.88 (d, J=8.6 Hz, 2H), 3.94 (s, 3H), 3.81-3.75 (m, 1H), 2.96-2.90 (m, 1H), 2.66-2.59 (m, 1H), 1.94-1.87 (m, 1H), 1.68-1.61 (m, 1H), 1.60-1.53 (m, 2H).

### Example 159. Synthesis of 3-(3,5-Difluoro-4-methoxyphenyl)-8-(2,6-dimethylpyridin-4-yl)-4-oxo-2-(pyrrolidin-2-yl)-3,4-dihydroquinazoline-6-carbonitrile (25fff)

Prepared as shown in Scheme 8b.

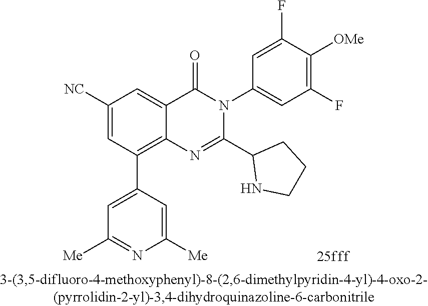 [SMILES: COc1c(F)cc(-n2c(C3CCCN3)nc3c(-c4cc(C)nc(C)c4)cc(C#N)cc3c2=O)cc1F]

The BOC-protected penultimate intermediate (53.1 mg, 0.0904 mmol) was deprotected in CH₂Cl₂ (1.0 mL) and trifluoroacetic acid (208 μL, 2.71 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 25fff (31.9 mg, 72%) as a white solid (LC/MS purity=98.2%). ¹H NMR (400 MHz, CDCl₃): δ=8.60 (d, J=1.8 Hz, 1H), 8.01 (d, J=1.8 Hz, 1H), 7.22 (s, 2H), 6.94 (d, J=10.3 Hz, 1H), 6.87 (d, J=10.3 Hz, 1H), 4.12 (s, 3H), 3.90 (t, J=6.7 Hz, 1H), 3.06 (dt, J=11.4, 6.0 Hz, 1H), 2.79 (dt, J=10.7, 6.4 Hz, 1H), 2.63 (s, 6H), 2.543 (brs), 1.79 (m, 4H).

### Example 160. Synthesis of 3-(3,5-Difluoro-4-methoxyphenyl)-8-(2,6-dimethylpyridin-4-yl)-2-((4S)-4-fluoropyrrolidin-2-yl)-4-oxo-3,4-dihydroquinazoline-6-carbonitrile (25ggg)

Prepared as shown in Scheme 8b.

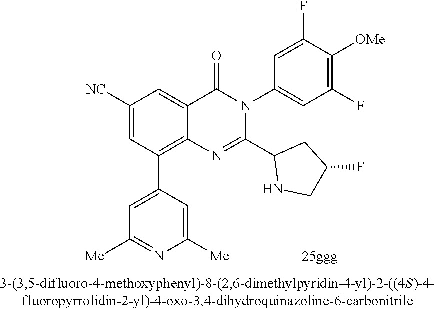 [SMILES: COc1c(F)cc(-n2c(C3C[C@H](F)CN3)nc3c(-c4cc(C)nc(C)c4)cc(C#N)cc3c2=O)cc1F]

The BOC-protected penultimate intermediate (70.3 mg, 0.116 mmol) was deprotected in CH₂Cl₂ (1.2 mL) and trifluoroacetic acid (266 μL, 3.48 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 25ggg (49.6 mg, 85%) as a white solid (LC/MS purity=99.4%). ¹H NMR (400 MHz, CDCl₃): δ=8.61 (d, J=1.5 Hz, 1H), 8.00 (d, J=1.8 Hz, 1H), 7.18 (s, 2H), 7.05 (d, J=10.4 Hz, 1H), 6.85 (d, J=10.2 Hz, 1H), 5.18 (d, J=54.0 Hz, 1H), 4.21 (t, J=7.4 Hz, 1H), 4.13 (s, 3H), 3.19-2.93 (m, 2H), 2.62 (s, 6H), 2.46-2.29 (m, 1H), 2.41 (brs, 1H), 2.19-2.07 (m, 1H).

### Example 161. Synthesis of 3-(3,5-Difluoro-4-(2-methoxyethoxy)phenyl)-8-(2-methylpyridin-4-yl)-4-oxo-2-(pyrrolidin-2-yl)-3,4-dihydroquinazoline-6-carbonitrile (25 hhh)

Prepared as shown in Scheme 8b.

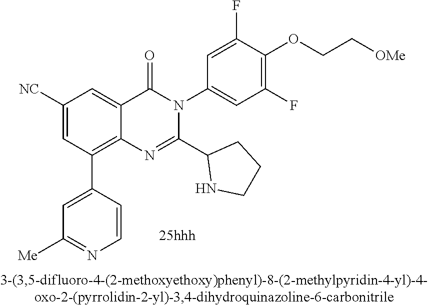 [SMILES: COCCOc1c(F)cc(-n2c(C3CCCN3)nc3c(-c4ccnc(C)c4)cc(C#N)cc3c2=O)cc1F]

The BOC-protected penultimate intermediate (137 mg, 0.265 mmol) was deprotected in CH₂Cl₂ (2.7 mL) and trifluoroacetic acid (609 μL, 7.95 mmol) and 4 M HCl in dioxane (343 μL, 1.37 mmol. The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 25 hhh (79.3 mg, 58%) as a white solid (LC/MS purity=99.3%). ¹H NMR (400 MHz, CDCl₃): δ=8.63 (d, J=5.1 Hz, 1H), 8.60 (d, J=1.4 Hz, 1H), 8.01 (s, 1H), 7.41 (s, 1H), 7.34 (d, J=5.0 Hz, 1H), 6.95 (d, J=10.1 Hz, 1H), 6.88 (d, J=10.1 Hz, 1H), 4.52-4.35 (m, 2H), 3.89 (t, J=6.8 Hz, 1H), 3.80-3.73 (m, 2H), 3.45 (s, 3H), 3.13-3.00 (m, 1H), 2.82-2.73 (m, 1H), 2.67 (s, 3H), 2.51 (brs, 1H), 1.85-1.62 (m, 4H).

### Example 162. Synthesis of 3-(3,5-difluoro-4-(2-methoxyethoxy)phenyl)-8-(2,6-dimethylpyridin-4-yl)-4-oxo-2-(pyrrolidin-2-yl)-3,4-dihydroquinazoline-6-carbonitrile (25iii)

Prepared as shown in Scheme 8b.

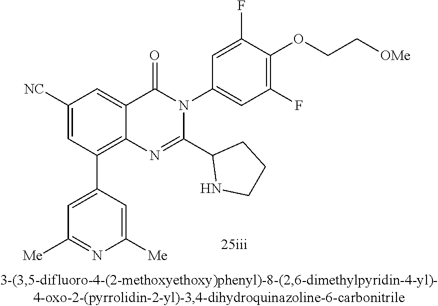 [SMILES: COCCOc1c(F)cc(-n2c(C3CCCN3)nc3c(-c4cc(C)nc(C)c4)cc(C#N)cc3c2=O)cc1F]

The BOC-protected penultimate intermediate (98.1 mg, 0.185 mmol) was deprotected in CH₂Cl₂ (1.9 mL) and trifluoroacetic acid (609 μL, 7.95 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 25iii (70.6 mg, 72%) as a white solid (LC/MS purity=99.4%). ¹H NMR (400 MHz, CDCl₃): δ=8.60 (d, J=1.8 Hz, 1H), 8.00 (d, J=1.8 Hz, 1H), 7.22 (s, 2H), 6.94 (d, J=10.2 Hz, 1H), 6.88 (d, J=10.1 Hz, 1H), 4.44-4.38 (m, 2H), 3.89 (t, J=6.8 Hz, 1H), 3.80-3.68 (m, 2H), 3.45 (s, 3H), 3.11-2.99 (m, 1H), 2.84-2.74 (m, 1H), 2.63 (s, 6H), 2.49 (brs, 1H), 1.86-1.65 (m, 4H).

### Example 163. Synthesis of 3-(3,5-difluoro-4-(2-oxa-6-azaspiro[3.3]heptan-6-yl)phenyl)-8-(2,6-dimethylpyridin-4-yl)-4-oxo-2-(pyrrolidin-2-yl)-3,4-dihydroquinazoline-6-carbonitrile (25jjj)

Prepared as shown in Scheme 8b.

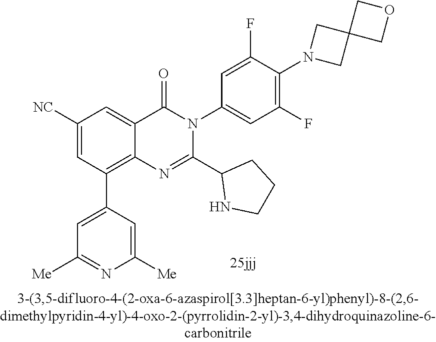 [SMILES: Cc1cc(-c2cc(C#N)cc3c(=O)n(-c4cc(F)c(N5CC6(COC6)C5)c(F)c4)c(C4CCCN4)nc23)cc(C)n1]

The BOC-protected penultimate intermediate (71.3 mg, 0.129 mmol) was deprotected in CH₂Cl₂ (1.3 mL) and trifluoroacetic acid (296 μL, 3.87 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 25jjj (22.6 mg, 32%) as a white solid (LC/MS purity=97.0%). ¹H NMR (400 MHz, CDCl₃): δ=8.59 (d, J=1.7 Hz, 1H), 7.99 (d, J=1.7 Hz, 1H), 7.21 (s, 2H), 6.76 (d, J=11.9 Hz, 1H), 6.70 (d, J=11.9 Hz, 1H), 4.85 (s, 4H), 4.45 (s, 4H), 4.04-3.83 (m, 1H), 3.18-2.96 (m, 1H), 2.84-2.71 (m, 1H), 2.76 (brs, 1H), 2.62 (s, 6H), 1.86-1.64 (m, 4H).

### Example 164. Synthesis of 3-(3,5-difluoro-4-(((3-(hydroxymethyl)oxetan-3-yl)methyl)amino)phenyl)-8-(2,6-dimethylpyridin-4-yl)-4-oxo-2-(pyrrolidin-2-yl)-3,4-dihydroquinazoline-6-carbonitrile (25kkk)

Prepared as shown in Scheme 8b.

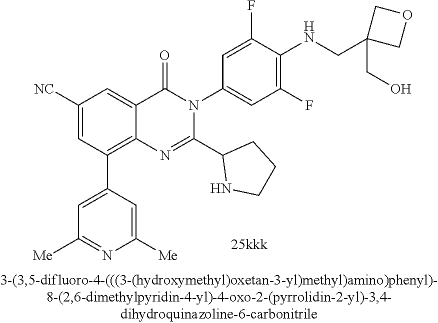 [SMILES: Cc1cc(-c2cc(C#N)cc3c(=O)n(-c4cc(F)c(NCC5(CO)COC5)c(F)c4)c(C4CCCN4)nc23)cc(C)n1]

3-(3,5-difluoro-4-(((3-(hydroxymethyl)oxetan-3-yl)methyl)amino)phenyl)-8-(2,6-dimethylpyridin-4-yl)-4-oxo-2-(pyrrolidin-2-yl)-3,4-dihydroquinazoline-6-carbonitrile (25kkk) was obtained as a side product from the reaction affording 25jjj, and was purified by CombiFlash (MeOH:CH₂Cl₂=5:100) to give 25kkk (22.1 mg, 30%) as a white solid (LC/MS purity=97.0%). ¹H NMR (400 MHz, CDCl₃): δ=8.60 (d, J=1.7 Hz, 1H), 8.00 (d, J=1.7 Hz, 1H), 7.22 (s, 2H), 6.82 (d, J=11.5 Hz, 1H), 6.77 (d, J=11.5 Hz, 1H), 4.74 (brs, 1H), 4.57 (d, J=6.2 Hz, 2H), 4.49 (d, J=6.2 Hz, 2H), 4.05 (s, 2H), 4.00-3.90 (m, 1H), 3.85 (d, J=5.7 Hz, 2H), 3.13-3.02 (m, 1H), 2.92 (brs, 1H), 2.84-2.74 (m, 1H), 2.63 (s, 6H), 1.90-1.69 (m, 4H).

### Example 165. Synthesis of 2-(2-azabicyclo[2.1.1]hexan-1-yl)-3-(2-methoxypyrimidin-5-yl)-8-(6-methylpyridin-3-yl)-4-oxo-3,4-dihydroquinazoline-6-carbonitrile (25lll)

Prepared as shown in Scheme 8b.

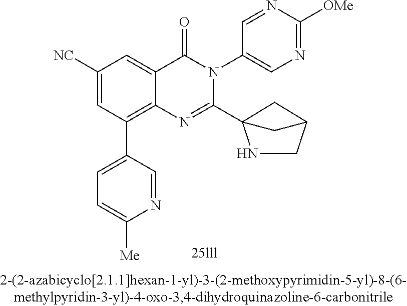 [SMILES: COc1ncc(-n2c(C34CC(CN3)C4)nc3c(-c4ccc(C)nc4)cc(C#N)cc3c2=O)cn1]

The BOC-protected penultimate intermediate (27.6 mg, 0.0500 mmol) was deprotected in CH₂Cl₂ (0.5 mL) and trifluoroacetic acid (115 μL, 1.50 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 25lll (14.2 mg, 63%) as a white solid (LC/MS purity=99.8%). ¹H NMR (400 MHz, CDCl₃): δ=8.81 (d, J=2.2 Hz, 1H), 8.61 (d, J=1.9 Hz, 1H), 8.55 (s, 2H), 8.02 (d, J=1.9 Hz, 1H), 7.87 (dd, J=8.0, 2.2 Hz, 1H), 7.28 (d, J=8.2 Hz, 1H), 4.10 (s, 3H), 2.84 (s, 2H), 2.65 (s, 3H), 2.57 (t, J=2.9 Hz, 1H), 1.95-1.78 (m, 2H), 1.71 (brs, 1H), 1.00 (dd, J=4.5, 1.7 Hz, 2H).

### Example 166. Synthesis of 2-(2-azabicyclo[2.1.1]hexan-1-yl)-8-(6-methylpyridin-3-yl)-4-oxo-3-(4-(trifluoromethoxy)phenyl)-3,4-dihydroquinazoline-6-carbonitrile (25mmm)

Prepared as shown in Scheme 8b

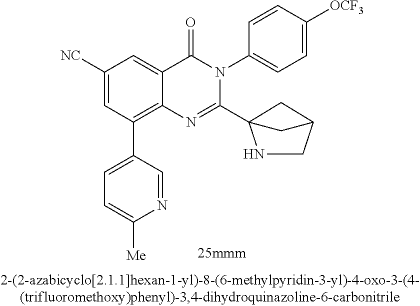 [SMILES: Cc1ccc(-c2cc(C#N)cc3c(=O)n(-c4ccc(OC(F)(F)F)cc4)c(C45CC(CN4)C5)nc23)cn1]

The BOC-protected penultimate intermediate (27.6 mg, 0.0320 mmol) was deprotected in CH₂Cl₂ (0.3 mL) and trifluoroacetic acid (73.5 μL, 0.96 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 25mmm (10.0 mg, 62%) as a white solid (LC/MS purity=98.9%). ¹H NMR (400 MHz, CDCl₃): δ=8.81 (d, J=2.1 Hz, 1H), 8.60 (d, J=1.8 Hz, 1H), 8.00 (d, J=1.8 Hz, 1H), 7.88 (dd, J=8.0, 2.3 Hz, 1H), 7.44 (d, J=8.8 Hz, 2H), 7.37 (d, J=8.7 Hz, 2H), 7.28 (d, J=8.2 Hz, 1H), 2.81 (s, 2H), 2.65 (s, 3H), 2.53 (t, J=2.7 Hz, 1H), 1.83 (brs, 1H), 1.78-1.71 (m, 2H), 0.96-0.85 (m, 2H).

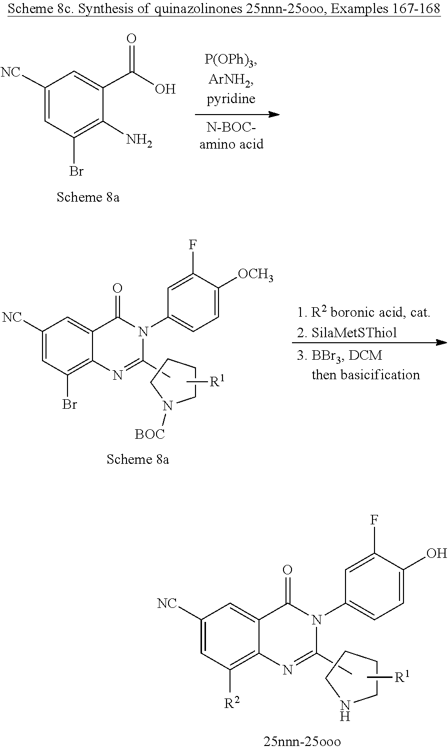

### Example 167. Synthesis of (R)-3-(3-fluoro-4-hydroxyphenyl)-8-(6-methylpyridin-3-yl)-4-oxo-2-(pyrrolidin-2-yl)-3,4-dihydroquinazoline-6-carbonitrile (25nnn)

Prepared as shown in Scheme 8c.

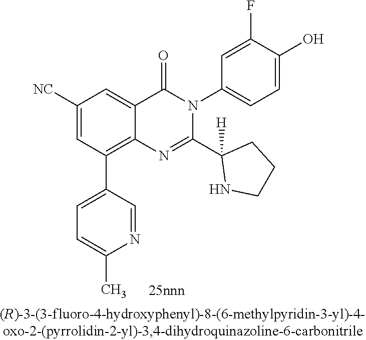 [SMILES: Cc1ccc(-c2cc(C#N)cc3c(=O)n(-c4ccc(O)c(F)c4)c([C@H]4CCCN4)nc23)cn1]

### Synthesis of (R)-3-(3-fluoro-4-hydroxyphenyl)-8-(6-methylpyridin-3-yl)-4-oxo-2-(pyrrolidin-2-yl)-3,4-dihydroquinazoline-6-carbonitrile (25nnn)

A mixture of 22a (20.6 g, 38.0 mmol), cesium carbonate (24.8 g, 76.0 mmol), 2-methylpyridine-5-boronic acid (6.75 g, 49.0 mmol) and tetrakis(tri-phenylphosphine) palladium (0) (3.3 g, 2.85 mmol) in 1,4-dioxane (400 mL) and H₂O (80 mL) was degassed and flushed with nitrogen. The mixture was heated at 85° C. for 16 h under nitrogen atmosphere. After cooling to room temperature, the mixture was concentrated to remove dioxane, and diluted with H₂O (400 mL) and extracted with DCM (3×300 mL). The combined organic layers were washed with brine (500 mL), dried over anhydrous Na₂SO₄, and filtered, then the solvent was evaporated under reduced pressure to afford a pale-yellow oil. The crude product was purified with flash chromatography (40% to 80% A (DCM:ethyl acetate=1:4)/Hexane) to give the product, which was treated with a palladium scavenger (SiliaMetS Thiol, 5 g), stirred at 40° C. for 8 h and at rt for 16 h, and filtered through Celite pad to remove any residual metal from the coupling reaction. Repeating this procedure of removing Pd for three times. After concentration, the corresponding coupling product (15 g, Y %=71%) was obtained as a yellow solid. ¹H NMR (400 MHz, Chloroform-d, a mixture of rotamers caused by -Boc group and atropisomers caused by —F atom) δ 9.16-9.10 (m, 1H), 8.78-8.68 (m, 1H), 8.65-8.58 (m, 1H), 8.03-7.92 (m, 1H), 7.70-7.65 (m, 3H), 7.58-7.50 (m, 2H), 7.49-7.44 (m, 3H), 7.40-7.27 (m, 2H), 7.22-7.07 (m, 2H), 7.04-6.94 (m, 1H), 4.59-4.47 (m, 1H), 4.02-3.90 (m, 3H), 3.47-3.35 (m, 2H), 2.71-2.59 (m, 3H), 2.08-1.95 (m, 2H), 1.91-1.79 (m, 2H), 1.37-1.21 (m, 9H).

A solution of BBr₃ in DCM (2.70 mL, 1N, 2.70 mmol) was added to a solution of the corresponding coupling product (300 mg, 0.54 mol) described above in DCM (5 mL) at −78° C., then stirred at room temperature overnight. The reaction mixture was quenched by 50 g ice and adjusted to pH 9 with saturated solutions of NaHCO₃. The aqueous layer was extracted with DCM (3×50 mL). The combined organic layers were washed with brine (100 mL), dried over anhydrous Na₂SO₄, and filtered, then the solvent was evaporated under reduced pressure. The residue was purified by flash chromatography (30-80% A (DCM:MeOH:NH₄OH=15:1:0.15)/n-hexane) to afford 25nnn (100 mg, 42%) as pale yellow solid. ¹H NMR (400 MHz, DMSO-d₆, a mixture of atropisomers (˜1:1 ratio) caused by —F atom) δ 8.87-8.71 (m, 1H), 8.53 (s, 1H), 8.44-8.29 (m, 1H), 8.11-7.97 (m, 1H), 7.49-7.28 (m, 2H), 7.20-7.03 (m, 2H), 3.88-3.76 (m, 1H), 2.95-2.85 (m, 1H), 2.72-2.64 (m, 2H), 2.58-2.55 (m, 3H), 1.97-1.84 (m, 1H), 1.73-1.47 (m, 3H). MS (ESI) m/z: 442.0 (M+1)⁺.

### Example 168. Synthesis of (R)-3-(3-fluoro-4-hydroxyphenyl)-4-oxo-2-(pyrrolidin-2-yl)-8-(6-(trifluoromethyl)pyridin-3-yl)-3,4-dihydroquinazoline-6-carbonitrile (25ooo)

Prepared as shown in Scheme 8c.

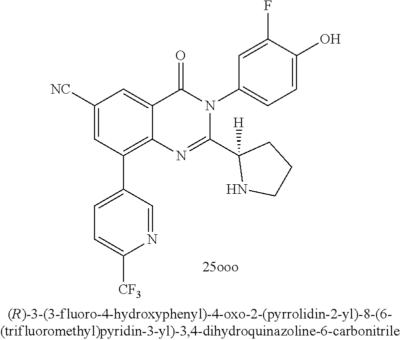 [SMILES: N#Cc1cc(-c2ccc(C(F)(F)F)nc2)c2nc([C@H]3CCCN3)n(-c3ccc(O)c(F)c3)c(=O)c2c1]

### Synthesis of (R)-3-(3-fluoro-4-hydroxyphenyl)-4-oxo-2-(pyrrolidin-2-yl)-8-(6-(trifluoromethyl)pyridin-3-yl)-3,4-dihydroquinazoline-6-carbonitrile (25ooo)

A mixture of 22a (20 g, 0.037 mol), cesium carbonate (24.11 g, 0.074 mol), (6-(trifluoromethyl)pyridin-3-yl)boronic acid (7.73 g, 0.040 mol) and tetrakis(tri-phenylphosphine) palladium (0) (0.85 g, 0.74 mmol) in 1,4-dioxane (400 mL) and H₂O (80 mL) was degassed and flushed with nitrogen. The mixture was heated at 85° C. for 16 h under nitrogen atmosphere. After cooling to room temperature, the mixture was concentrated to remove dioxane, and diluted with H₂O (400 mL) and extracted with DCM (3×300 mL). The combined organic layers were washed with brine (500 mL), dried over anhydrous Na₂SO₄, and filtered, then the solvent was evaporated under reduced pressure to afford a pale-yellow oil. The crude product was purified with flash chromatography (0% to 30% A (DCM:ethyl acetate=1:4)/Hexane) to give the product, which was treated with a palladium scavenger (SiliaMetS Thiol, 4 g), stirred at 40° C. for 8 h and at rt for 16 h, and filtered through Celite pad to remove any residual metal from the coupling reaction. Repeating this procedure of removing Pd for three times. After concentration, the corresponding coupling product (13.0 g, 58%) was obtained as a yellow solid. ¹H NMR (400 MHz, Chloroform-d, a mixture of rotamers caused by -Boc group and atropisomers caused by —F atom) δ 9.01-8.82 (m, 1H), 8.71-8.61 (m, 1H), 8.33-8.11 (m, 1H), 8.10-7.99 (m, 1H), 7.91-7.78 (m, 1H), 7.39-7.29 (m, 1H), 7.24-7.08 (m, 1H), 7.07-6.94 (m, 1H), 4.62-4.46 (m, 1H), 4.07-3.91 (m, 3H), 3.46-3.26 (m, 2H), 2.08-1.76 (m, 4H), 1.32 (s, 9H).

A solution of BBr₃ in DCM (1.64 mL, 1N, 1.64 mmol) was added to a solution of the corresponding coupling product (200 mg, 0.33 mmol) described above in DCM (5 mL) at −78° C., then stirred at room temperature overnight. The reaction mixture was quenched by 50 g ice and adjusted to pH 9 with saturated solutions of NaHCO₃. The aqueous layer was extracted with DCM (3×50 mL). The combined organic layers were washed with brine (100 mL), dried over anhydrous Na₂SO₄, and filtered, then the solvent was evaporated under reduced pressure. The residue was purified by flash chromatography (30-75% A (DCM:MeOH:NH₄OH=15:1:0.15)/n-hexane) to afford 25ooo (80 mg, 49%) as a pale-yellow solid. ¹H NMR (400 MHz, DMSO-d₆, a mixture of atropisomers (≈1:1 ratio) caused by —F atom) δ 9.10 (s, 1H), 8.62 (s, 1H), 8.50 (s, 1H), 8.44 (d, J=8.2 Hz, 1H), 8.09 (d, J=8.2 Hz, 1H), 7.38 (d, J=11.4 Hz, 1H), 7.19-7.00 (m, 2H), 3.85-3.77 (m, 1H), 2.86-2.78 (m, 1H), 2.69-2.58 (m, 2H), 1.93-1.84 (m, 1H), 1.65-1.48 (m, 3H). MS (ESI) m/z: 496.0 (M+1)⁺.

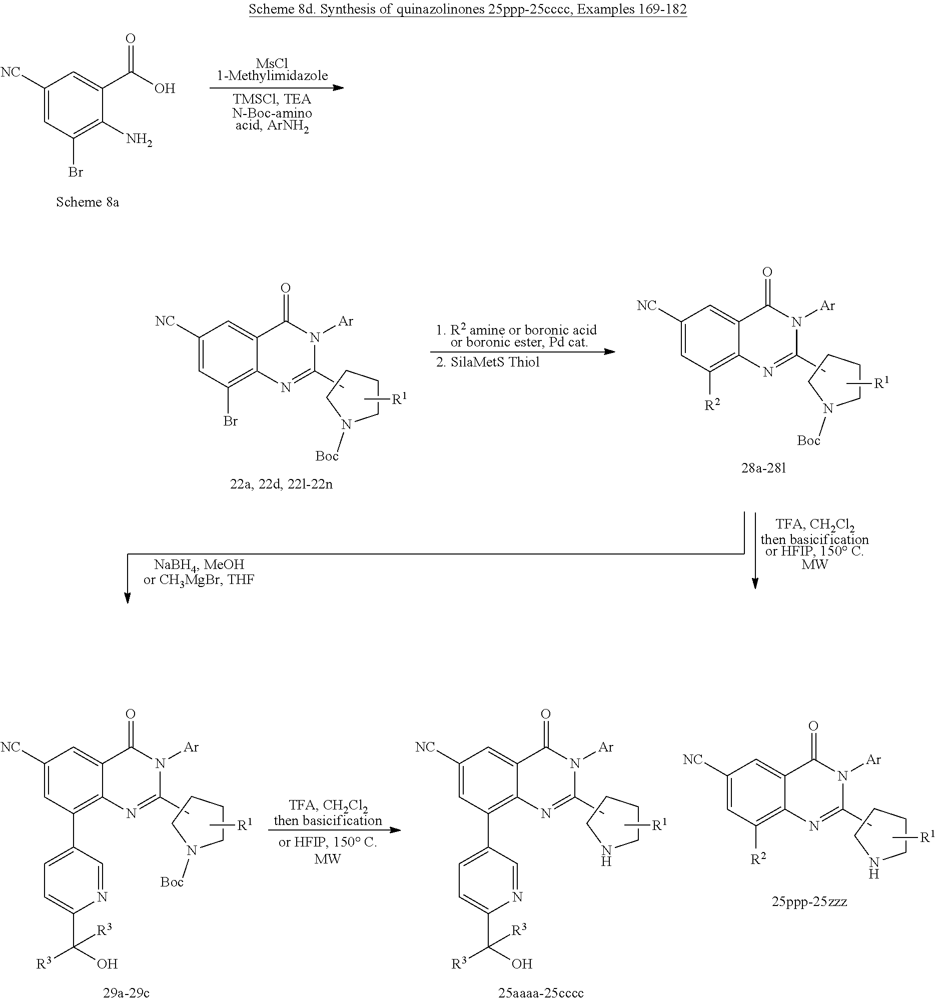

### Example 169. Synthesis of 3-(3,5-difluoro-4-methoxyphenyl)-8-(4-(3-hydroxyprop-1-yn-1-yl)phenyl)-4-oxo-2-(pyrrolidin-2-yl)-3,4-dihydroquinazoline-6-carbonitrile (25ppp)

Prepared as shown in Scheme 8d.

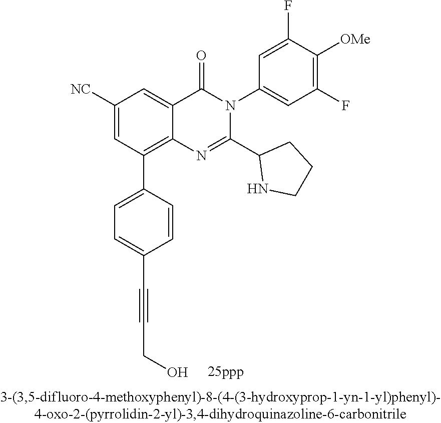 [SMILES: COc1c(F)cc(-n2c(C3CCCN3)nc3c(-c4ccc(C#CCO)cc4)cc(C#N)cc3c2=O)cc1F]

### Synthesis of tert-butyl 2-(8-bromo-6-cyano-3-(3,5-difluoro-4-methoxyphenyl)-4-oxo-3,4-dihydroquinazolin-2-yl)pyrrolidine-1-carboxylate (22l)

To the mixture of Boc-DL-proline (6.70 g, 0.031 mol) in anhydrous DCM (200 mL) under N₂ cooled to 0° C. was added dropwise 1-methylimidazole (5.77 mL, 0.073 mol) and MsCl (2.40 mL, 0.031 mol). After being stirred at rt for 1 h, 2-amino-3-bromo-5-cyanobenzoic acid (5.00 g, 0.021 mol) was added into the reaction mixture in one portion and stirred at rt for 16 h. Then, 3,5-difluoro-4-methoxyaniline (4.95 g, 0.031 mol) was added into the reaction mixture at rt and heated to reflux for 16 h. After being cooled to rt, triethylamine (26.00 mL, 0.186 mol) and TMSCl (18.4 mL, 0.145 mol) was added dropwise into the reaction mixture and stirred at rt for 24 h. The reaction mixture was diluted with DCM (400 mL), washed with 1N HCl, saturated aqueous Na₂CO₃, and brine in turn, dried over Na₂SO₄, and filtered, then the solvent was evaporated under reduced pressure. The residue was purified by flash chromatography (0% to 10% A (DCM:ethyl acetate=1:4)/Hexane) to afford 221 (7.00 g, 60%) as pale yellow solid. ¹H NMR (400 MHz, Chloroform-d, a mixture of rotamers caused by -Boc group) δ 8.55-8.45 (m, 1H), 8.27-8.13 (m, 1H), 7.26-7.19 (m, 1H), 6.88-6.73 (m, 1H), 4.57-4.45 (m, 1H), 4.17-4.06 (m, 3H), 3.98-3.77 (m, 1H), 3.63-3.41 (m, 1H), 2.32-2.03 (m, 2H), 1.96-1.85 (m, 2H), 1.45-1.26 (m, 10H).

### Synthesis of tert-butyl 2-(6-cyano-3-(3,5-difluoro-4-methoxyphenyl)-8-(4-(3-hydroxyprop-1-yn-1-yl)phenyl)-4-oxo-3,4-dihydroquinazolin-2-yl)pyrrolidine-1-carboxylate (28a)

A mixture of 221 (400 mg, 0.71 mmol), cesium carbonate (465 mg, 1.43 mmol), 3-(4-(4,4,5,5-tetramethyl-1,3,2-dioxaborolan-2-yl)phenyl)prop-2-yn-1-ol (239 mg, 0.93 mmol) and tetrakis(tri-phenylphosphine) palladium (0) (62 mg, 0.054 mmol) in 1,4-dioxane (8 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (20 mL) and extracted with DCM (3×50 mL). The combined organic layers were washed with brine (150 mL), dried over anhydrous Na₂SO₄, and filtered, then the solvent was evaporated under reduced pressure to afford a pale-yellow oil. The crude product was purified with flash chromatography (0% to 30% A (DCM:ethyl acetate=1:4)/Hexane) to give the product, which was treated with a palladium scavenger (SiliaMetS Thiol, 90 mg), stirred at 40° C. for 8 h and at rt for 16 h, and filtered through Celite pad to remove any residual metal from the coupling reaction. Repeating this procedure of removing Pd for three times. After concentration, 28a (260 mg, 64%) was obtained as a yellow solid. ¹H NMR (400 MHz, Chloroform-d, a mixture of rotamers caused by -Boc group) δ 8.60-8.52 (m, 1H), 8.05-7.95 (m, 1H), 7.63-7.50 (m, 4H), 7.26-7.22 (m, 1H), 6.99-6.79 (m, 1H), 4.60-4.43 (m, 3H), 4.17-4.04 (m, 3H), 3.46-3.30 (m, 2H), 2.01-1.76 (m, 4H), 1.47-1.28 (m, 9H).

### Synthesis of 3-(3,5-difluoro-4-methoxyphenyl)-8-(4-(3-hydroxyprop-1-yn-1-yl)phenyl)-4-oxo-2-(pyrrolidin-2-yl)-3,4-dihydroquinazoline-6-carbonitrile (25ppp)

Trifluoroacetic acid (0.79 mL, 10.28 mmol) was added to a solution of pale yellow solid 28a (210 mg, 0.34 mol) in DCM (10 mL) at 0° C., then stirred at room temperature overnight, then the solvent was diluted with CH₂Cl₂ (150 mL) and washed with saturated solutions of Na₂CO₃ (3×100 mL) and brine (150 mL), dried over anhydrous Na₂SO₄, and filtered, then the solvent was evaporated under reduced pressure. The residue was purified by flash chromatography (0% to 4% MeOH/CH₂Cl₂) to afford 25ppp (100 mg, 57%) as a yellow solid. ¹H NMR (400 MHz, Chloroform-d) δ 8.57 (s, 1H), 7.99 (s, 1H), 7.55 (s, 4H), 6.91 (dd, J=27.1, 9.7 Hz, 2H), 4.53 (s, 2H), 4.12 (s, 3H), 3.99-3.77 (m, 1H), 3.13-2.94 (m, 1H), 2.85-2.66 (m, 1H), 2.40 (s, 1H), 1.85-1.67 (m, 4H). MS (ESI) m/z: 456.1 (M+1)⁺.

### Example 170. Synthesis of 3-(3,5-difluoro-4-methoxyphenyl)-8-(4-(3-hydroxy-3-methylbut-1-yn-1-yl)phenyl)-4-oxo-2-(pyrrolidin-2-yl)-3,4-dihydroquinazoline-6-carbonitrile (25qqq)

Prepared as shown in Scheme 8d.

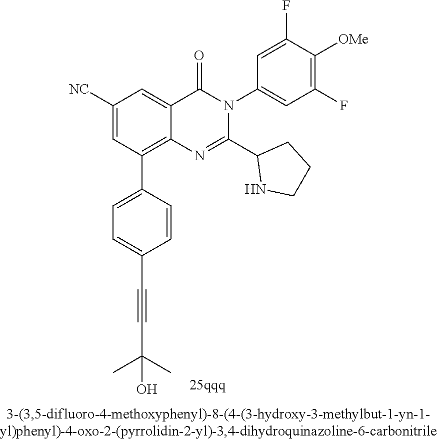 [SMILES: COc1c(F)cc(-n2c(C3CCCN3)nc3c(-c4ccc(C#CC(C)(C)O)cc4)cc(C#N)cc3c2=O)cc1F]

### Synthesis of tert-butyl 2-(6-cyano-3-(3,5-difluoro-4-methoxyphenyl)-8-(4-(3-hydroxy-3-methylbut-1-yn-1-yl)phenyl)-4-oxo-3,4-dihydroquinazolin-2-yl)pyrrolidine-1-carboxylate (28b)

The procedure leading to 28a was followed using 221 (400 mg, 0.71 mmol), cesium carbonate (465 mg, 1.43 mmol), 2-methyl-4-(4-(4,4,5,5-tetramethyl-1,3,2-dioxaborolan-2-yl)phenyl)but-3-yn-2-ol (265 mg, 0.93 mmol) and tetrakis(tri-phenylphosphine) palladium (0) (62 mg, 0.0535 mmol), 1,4-dioxane (10 mL), and H₂O (2 mL). Purification by flash chromatography (0% to 20% A (DCM:ethyl acetate=1:4)/Hexane) and a palladium scavenger (SiliaMetS Thiol, 3×50 mg) yielded 28b (140 mg, 31%) as a yellow solid. ¹H NMR (400 MHz, Chloroform-d, a mixture of rotamers caused by -Boc group) δ 8.62-8.52 (m, 1H), 8.05-7.95 (m, 1H), 7.62-7.48 (m, 4H), 7.24-6.96 (m, 1H), 6.88-6.74 (m, 1H), 4.58-4.43 (m, 1H), 4.17-4.06 (m, 3H), 3.51-3.33 (m, 2H), 2.01-1.70 (m, 4H), 1.71-1.57 (m, 6H), 1.44-1.26 (m, 9H).

### Synthesis of 3-(3,5-difluoro-4-methoxyphenyl)-8-(4-(3-hydroxy-3-methylbut-1-yn-1-yl)phenyl)-4-oxo-2-(pyrrolidin-2-yl)-3,4-dihydroquinazoline-6-carbonitrile (25qqq)

A solution of 28b (100 mg, 0.156 mmol) in hexafluoroisopropanol (4 mL) was heated at 160° C. with MWI for 2 h. The solvent was removed in vacuo and the residue was purified by flash chromatography (0% to 3% MeOH/DCM) to afford 25qqq (29 mg, 34%) as a yellow solid. ¹H NMR (400 MHz, Chloroform-d) δ 8.56 (s, 1H), 7.99 (s, 1H), 7.59-7.49 (m, 4H), 6.98-6.90 (m, 1H), 6.90-6.83 (m, 1H), 4.12 (s, 3H), 3.93-3.82 (m, 1H), 3.07 (dt, J=12.0, 6.2 Hz, 1H), 2.77-2.66 (m, 1H), 2.31 (s, 1H), 1.86-1.68 (m, 4H), 1.66 (s, 6H). MS (ESI) m/z: 541.0 (M+1)⁺.

### Example 171. Synthesis of 3-(3,5-difluoro-4-methoxyphenyl)-8-(4-(2-hydroxypropan-2-yl)phenyl)-4-oxo-2-(pyrrolidin-2-yl)-3,4-dihydroquinazoline-6-carbonitrile (25rrr)

Prepared as shown in Scheme 8d

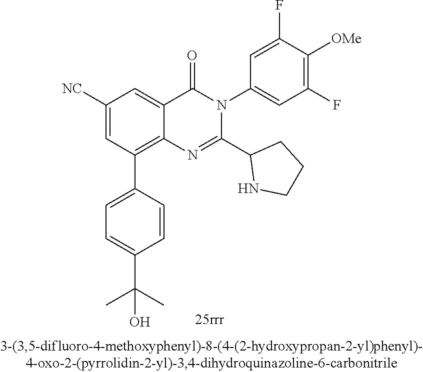 [SMILES: COc1c(F)cc(-n2c(C3CCCN3)nc3c(-c4ccc(C(C)(C)O)cc4)cc(C#N)cc3c2=O)cc1F]

### Synthesis of tert-butyl 2-(6-cyano-3-(3,5-difluoro-4-methoxyphenyl)-8-(4-(2-hydroxypropan-2-yl)phenyl)-4-oxo-3,4-dihydroquinazolin-2-yl)pyrrolidine-1-carboxylate (28c)

The procedure leading to 28a was followed using 221 (400 mg, 0.71 mmol), cesium carbonate (465 mg, 1.43 mmol), (4-(2-hydroxypropan-2-yl)phenyl)boronic acid (167 mg, 0.93 mmol) and tetrakis(tri-phenylphosphine) palladium (0) (62 mg, 0.0535 mmol), 1,4-dioxane (10 mL), and H₂O (2 mL). Purification by flash chromatography (0% to 30% A (DCM:ethyl acetate=1:4)/Hexane) and a palladium scavenger (SiliaMetS Thiol, 3×130 mg) yielded 28c (380 mg, 86%) as a yellow solid. ¹H NMR (400 MHz, Chloroform-d, a mixture of rotamers caused by -Boc group) δ 8.58-8.51 (m, 1H), 8.05-7.94 (m, 1H), 7.72-7.61 (m, 1H), 7.58-7.42 (m, 1H), 7.31-7.21 (m, 2H), 6.88-6.79 (m, 1H), 4.54-4.42 (m, 1H), 4.15-4.06 (m, 3H), 3.46-3.28 (m, 2H), 2.03-1.87 (m, 2H), 1.83-1.71 (m, 2H), 1.69-1.65 (m, 6H), 1.38-1.29 (m, 9H).

### Synthesis of 3-(3,5-difluoro-4-methoxyphenyl)-8-(4-(2-hydroxypropan-2-yl)phenyl)-4-oxo-2-(pyrrolidin-2-yl)-3,4-dihydroquinazoline-6-carbonitrile (25rrr)

A solution of 28c (310 mg, 0.502 mmol) in hexafluoroisopropanol (5 mL) was heated at 150° C. with MWI for 3 h. The solvent was removed in vacuo and the residue was purified by flash chromatography (0% to 1.8% MeOH/DCM) to afford 25rrr (64 mg, Y %=25%, LC/MS purity=95.28%) as a yellow solid. ¹H NMR (400 MHz, Chloroform-d) δ 8.56 (s, 1H), 8.00 (s, 1H), 7.71-7.50 (m, 4H), 6.91 (dd, J=21.2, 10.4 Hz, 2H), 4.12 (s, 3H), 3.95-3.80 (m, 1H), 3.10-2.98 (m, 1H), 2.79-2.68 (m, 1H), 1.85-1.69 (m, 4H), 1.66 (s, 6H). MS (ESI) m/z: 517.0 (M+1)⁺.

### Example 172. Synthesis of 3-(3,5-difluoro-4-methoxyphenyl)-4-oxo-8-(4-(prop-1-en-2-yl)phenyl)-2-(pyrrolidin-2-yl)-3,4-dihydroquinazoline-6-carbonitrile (25sss)

Prepared as shown in Scheme 8d.

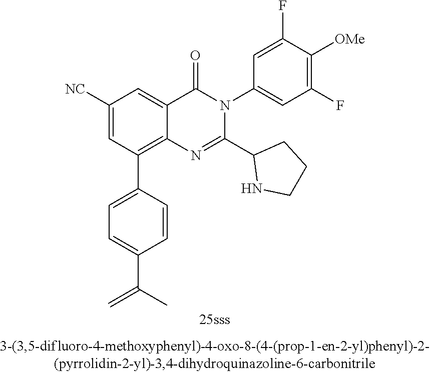 [SMILES: C=C(C)c1ccc(-c2cc(C#N)cc3c(=O)n(-c4cc(F)c(OC)c(F)c4)c(C4CCCN4)nc23)cc1]

### Synthesis of 3-(3,5-difluoro-4-methoxyphenyl)-4-oxo-8-(4-(prop-1-en-2-yl)phenyl)-2-(pyrrolidin-2-yl)-3,4-dihydroquinazoline-6-carbonitrile (25sss)

A solution of 28c (310 mg, 0.502 mmol) in hexafluoroisopropanol (5 mL) was heated at 150° C. with MWI for 3 h. The solvent was removed in vacuo and the residue was purified by flash chromatography (0% to 1.8% MeOH/DCM) to afford 25sss (50 mg, 20%) as a yellow solid. ¹H NMR (400 MHz, Chloroform-d) δ 8.56 (s, 1H), 8.01 (s, 1H), 7.59 (s, 4H), 6.91 (dd, J=22.9, 10.0 Hz, 2H), 5.50 (s, 1H), 5.19 (s, 1H), 4.12 (s, 3H), 3.93-3.79 (m, 1H), 3.12-2.99 (m, 1H), 2.80-2.67 (m, 1H), 2.23 (s, 3H), 1.83-1.66 (m, 4H). MS (ESI) m/z: 499.0 (M+1)⁺.

### Example 173. Synthesis of 2-(2-azabicyclo[2.1.1]hexan-1-yl)-3-(3,5-difluoro-4-methoxyphenyl)-8-(2-methylpyridin-4-yl)-4-oxo-3,4-dihydroquinazoline-6-carbonitrile (25ttt)

Prepared as shown in Scheme 8d.

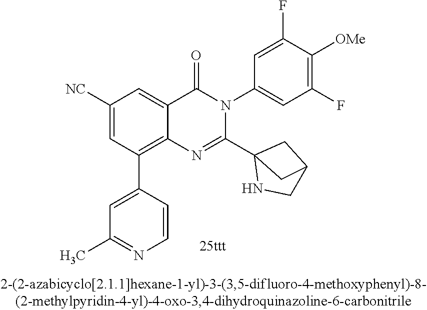 [SMILES: COc1c(F)cc(-n2c(C34CC(CN3)C4)nc3c(-c4ccnc(C)c4)cc(C#N)cc3c2=O)cc1F]

### Synthesis of tert-butyl 1-(8-bromo-6-cyano-3-(3,5-difluoro-4-methoxyphenyl)-4-oxo-3,4-dihydroquinazolin-2-yl)-2-azabicyclo[2.1.1]hexane-2-carboxylate (22m)

The procedure leading to 221 was followed using 2-amino-3-bromo-5-cyanobenzoic acid (2.97 g, 0.012 mol), 2-(tert-butoxycarbonyl)-2-azabicyclo[2.1.1]hexane-1-carboxylic acid (4.20 g, 0.019 mmol), 1-methylimidazole (11.80 mL, 0.15 mol), MsCl (1.43 mL, 0.019 mol), 3,5-difluoro-4-methoxyaniline (4.90 g, 0.031 mmol), triethylamine (15.40 mL, 0.111 mol), TMSCl (11.00 mL, 0.086 mol), and anhydrous DCM (150 mL). Purification by flash chromatography (0% to 5% A (DCM:ethyl acetate=1:1)/Hexane) yielded 22m (3.00 g, 43%) as a pale yellow solid. ¹H NMR (400 MHz, Chloroform-d, a mixture of rotamers caused by -Boc group) δ 8.51 (d, J=1.8 Hz, 1H), 8.21 (s, 1H), 7.22 (s, 1H), 6.74 (d, J=10.3 Hz, 1H), 4.09 (s, 3H), 3.62-3.35 (m, 1H), 3.21-3.08 (m, 1H), 2.70-2.60 (m, 1H), 2.53-2.34 (m, 1H), 2.09-1.91 (m, 2H), 1.42 (s, 9H), 0.96-0.79 (m, 1H).

### Synthesis of tert-butyl 1-(6-cyano-3-(3,5-difluoro-4-methoxyphenyl)-8-(2-methylpyridin-4-yl)-4-oxo-3,4-dihydroquinazolin-2-yl)-2-azabicyclo[2.1.1]hexane-2-carboxylate (28d)

The procedure leading to 28a was followed using 22m (275 mg, 0.48 mmol), cesium carbonate (316 mg, 0.96 mmol), (2-methylpyridin-4-yl)boronic acid (85 mg, 0.62 mmol) and tetrakis(tri-phenylphosphine) palladium (0) (42 mg, 0.036 mmol), 1,4-dioxane (8 mL), and H₂O (2 mL). Purification by flash chromatography (0% to 40% A (DCM:ethyl acetate=1:4)/Hexane) and a palladium scavenger (SiliaMetS Thiol, 3×80 mg) yielded 28d (220 mg, 78%) as a white solid. ¹H NMR (400 MHz, Chloroform-d, a mixture of rotamers caused by -Boc group) δ 8.62 (d, J=2.0 Hz, 1H), 8.59 (d, J=5.3 Hz, 1H), 8.02 (s, 1H), 7.72-7.61 (m, 1H), 7.57-7.44 (m, 2H), 7.39 (d, J=4.9 Hz, 1H), 7.22 (d, J=11.0 Hz, 1H), 6.79 (dt, J=10.4, 2.1 Hz, 1H), 4.09 (s, 3H), 3.48-3.34 (m, 1H), 3.17-3.06 (m, 1H), 2.64 (s, 3H), 2.64-2.57 (m, 2H), 2.20-2.12 (m, 1H), 1.98-1.91 (m, 1H), 1.75 (dd, J=10.1, 8.0 Hz, 1H), 1.55-1.19 (m, 9H), 0.88-0.76 (m, 1H).

### Synthesis of 2-(2-azabicyclo[2.1.1]hexan-1-yl)-3-(3,5-difluoro-4-methoxyphenyl)-8-(2-methylpyridin-4-yl)-4-oxo-3,4-dihydroquinazoline-6-carbonitrile (25ttt)

Trifluoroacetic acid (0.83 mL, 10.76 mmol) was added to a solution of 28d (210 mg, 0.36 mmol) in DCM (10 mL) at 0° C., then stirred at room temperature overnight, then the solvent was diluted with DCM (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 3% MeOH/DCM) to afford 25ttt (90 mg, 52%) as a white solid. ¹H NMR (400 MHz, Chloroform-d) δ 8.63 (s, 1H), 8.60 (d, J=5.1 Hz, 1H), 8.03 (s, 1H), 7.46 (s, 1H), 7.38 (d, J=4.9 Hz, 1H), 7.01 (d, J=7.9 Hz, 2H), 4.10 (s, 3H), 2.86 (s, 2H), 2.65 (s, 3H), 2.61-2.57 (m, 1H), 1.84-1.78 (m, 2H), 1.76-1.60 (m, 2H), 1.07-1.00 (m, 2H). MS (ESI) m/z: 486.0 (M+1)⁺.

### Example 174. Synthesis of 2-(2-azabicyclo[2.1.1]hexan-1-yl)-3-(3,5-difluoro-4-methoxyphenyl)-8-(2,6-dimethylpyridin-4-yl)-4-oxo-3,4-dihydroquinazoline-6-carbonitrile (25uuu)

Prepared as shown in Scheme 8d.

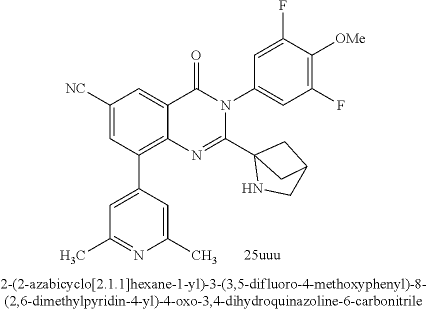 [SMILES: COc1c(F)cc(-n2c(C34CC(CN3)C4)nc3c(-c4cc(C)nc(C)c4)cc(C#N)cc3c2=O)cc1F]

### Synthesis of tert-butyl 1-(6-cyano-3-(3,5-difluoro-4-methoxyphenyl)-8-(2,6-dimethylpyridin-4-yl)-4-oxo-3,4-dihydroquinazolin-2-yl)-2-azabicyclo[2.1.1]hexane-2-carboxylate (28e)

The procedure leading to 28a was followed using 22m (275 mg, 0.48 mmol), cesium carbonate (316 mg, 0.96 mmol), (2,6-dimethylpyridin-4-yl)boronic acid (94 mg, 0.62 mmol) and tetrakis(tri-phenylphosphine) palladium (0) (42 mg, 0.036 mmol), 1,4-dioxane (8 mL), and H₂O (2 mL). Purification by flash chromatography (0% to 50% A (DCM:ethyl acetate=1:4)/Hexane) and a palladium scavenger (SiliaMetS Thiol, 3×80 mg) yielded 28e (230 mg, 80%) as a white solid. ¹H NMR (400 MHz, Chloroform-d, a mixture of rotamers caused by -Boc group) δ 8.65-8.57 (m, 1H), 8.01 (s, 1H), 7.74-7.63 (m, 1H), 7.62-7.36 (m, 2H), 7.25-7.15 (m, 1H), 6.84-6.73 (m, 1H), 4.09 (s, 3H), 3.51-3.34 (m, 1H), 3.17-3.04 (m, 1H), 2.60 (s, 6H), 2.23-2.12 (m, 1H), 1.94 (s, 1H), 1.79-1.69 (m, 1H), 1.40 (s, 9H), 0.90-0.71 (m, 1H).

### Synthesis of 2-(2-azabicyclo[2.1.1]hexan-1-yl)-3-(3,5-difluoro-4-methoxyphenyl)-8-(2,6-dimethylpyridin-4-yl)-4-oxo-3,4-dihydroquinazoline-6-carbonitrile (25uuu)

Trifluoroacetic acid (0.84 mL, 11.00 mmol) was added to a solution of 28e (220 mg, 0.37 mmol) in DCM (10 mL) at 0° C., then stirred at room temperature overnight, then the solvent was diluted with DCM (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 3% MeOH/DCM) to afford 25uuu (105 mg, 57%) as a white solid. ¹H NMR (400 MHz, Chloroform-d) δ 8.61 (d, J=2.0 Hz, 1H), 8.03 (d, J=2.0 Hz, 1H), 7.27 (s, 2H), 7.05-6.96 (m, 2H), 4.10 (s, 3H), 2.86 (s, 2H), 2.61 (s, 6H), 2.60-2.58 (m, 1H), 1.83-1.79 (m, 2H), 1.70-1.60 (m, 2H), 1.06-0.98 (m, 2H). MS (ESI) m/z: 500.0 (M+1)⁺.

### Example 175. Synthesis of 2-(2-azabicyclo[2.1.1]hexan-1-yl)-3-(3,5-difluoro-4-methoxyphenyl)-8-(6-methylpyridin-3-yl)-4-oxo-3,4-dihydroquinazoline-6-carbonitrile (25vvv)

Prepared as shown in Scheme 8d.

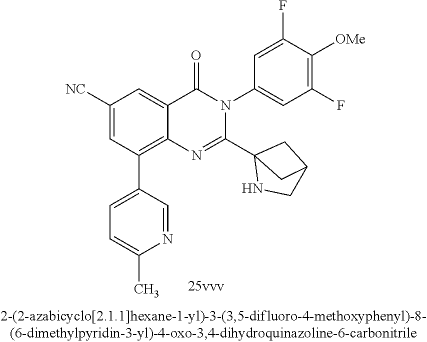 [SMILES: COc1c(F)cc(-n2c(C34CC(CN3)C4)nc3c(-c4ccc(C)nc4)cc(C#N)cc3c2=O)cc1F]

### Synthesis of tert-butyl 1-(6-cyano-3-(3,5-difluoro-4-methoxyphenyl)-8-(6-methylpyridin-3-yl)-4-oxo-3,4-dihydroquinazolin-2-yl)-2-azabicyclo[2.1.1]hexane-2-carboxylate (28f)

The procedure leading to 28a was followed using 22m (250 mg, 0.44 mmol), cesium carbonate (287 mg, 0.87 mmol), (6-methylpyridin-3-yl)boronic acid (78 mg, 0.57 mmol) and tetrakis(tri-phenylphosphine) palladium (0) (38 mg, 0.033 mmol), 1,4-dioxane (8 mL), and H₂O (2 mL). Purification by flash chromatography (0% to 55% A (DCM:ethyl acetate=1:4)/Hexane) and a palladium scavenger (SiliaMetS Thiol, 3×50 mg) yielded 28f (160 mg, 63%) as a white solid. ¹H NMR (400 MHz, Chloroform-d, a mixture of rotamers caused by -Boc group) δ 8.83 (d, J=1.7 Hz, 1H), 8.59 (d, J=2.0 Hz, 1H), 7.99 (s, 1H), 7.86 (dd, J=8.1, 2.3 Hz, 1H), 7.72-7.62 (m, 1H), 7.58-7.44 (m, 1H), 7.29-7.26 (m, 1H), 7.23 (s, 1H), 6.78 (dt, J=10.5, 2.1 Hz, 1H), 4.09 (s, 3H), 3.39 (s, 1H), 3.16-3.06 (m, 1H), 2.64 (s, 3H), 2.59 (s, 1H), 2.25-2.12 (m, 1H), 2.01-1.89 (m, 1H), 1.86-1.74 (m, 1H), 1.56-1.16 (m, 9H), 0.90-0.70 (m, 1H).

### Synthesis of 2-(2-azabicyclo[2.1.1]hexan-1-yl)-3-(3,5-difluoro-4-methoxyphenyl)-8-(6-methylpyridin-3-yl)-4-oxo-3,4-dihydroquinazoline-6-carbonitrile (25vvv)

Trifluoroacetic acid (0.84 mL, 11.00 mmol) was added to a solution of 28f (220 mg, 0.37 mmol) in DCM (10 mL) at 0° C., then stirred at room temperature overnight, then the solvent was diluted with DCM (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 3% MeOH/DCM) to afford 25vvv (105 mg, 57%) as a white solid. ¹H NMR (400 MHz, Chloroform-d) δ 8.80 (s, 1H), 8.60 (d, J=1.7 Hz, 1H), 8.00 (d, J=1.7 Hz, 1H), 7.87 (dd, J=7.9, 2.0 Hz, 1H), 7.28 (s, 1H), 7.02 (d, J=8.1 Hz, 2H), 4.10 (s, 3H), 2.85 (s, 2H), 2.65 (s, 3H), 2.57 (s, 1H), 1.79 (s, 2H), 1.71 (s, 1H), 1.07-0.98 (m, 2H). MS (ESI) m/z: 486.0 (M+1)⁺.

### Example 176. Synthesis of 2-(2-azabicyclo[2.1.1]hexan-1-yl)-3-(3,5-difluoro-4-methoxyphenyl)-8-(5,6-dimethylpyridin-3-yl)-4-oxo-3,4-dihydroquinazoline-6-carbonitrile (25www)

Prepared as shown in Scheme 8d.

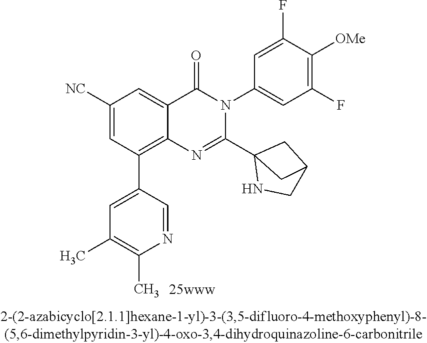 [SMILES: COc1c(F)cc(-n2c(C34CC(CN3)C4)nc3c(-c4cnc(C)c(C)c4)cc(C#N)cc3c2=O)cc1F]

### Synthesis of tert-butyl 1-(6-cyano-3-(3,5-difluoro-4-methoxyphenyl)-8-(5,6-dimethylpyridin-3-yl)-4-oxo-3,4-dihydroquinazolin-2-yl)-2-azabicyclo[2.1.1]hexane-2-carboxylate (28g)

The procedure leading to 28a was followed using 22m (250 mg, 0.44 mmol), cesium carbonate (287 mg, 0.87 mmol), 2,3-dimethyl-5-(4,4,5,5-tetramethyl-1,3,2-dioxaborolan-2-yl)pyridine (132 mg, 0.57 mmol) and tetrakis(tri-phenylphosphine) palladium (0) (38 mg, 0.033 mmol), 1,4-dioxane (8 mL), and H₂O (2 mL). Purification by flash chromatography (0% to 50% A (DCM:ethyl acetate=1:4)/Hexane) and a palladium scavenger (SiliaMetS Thiol, 3×40 mg) yielded 28g (110 mg, 42%) as a white solid. ¹H NMR (400 MHz, Chloroform-d, a mixture of rotamers caused by -Boc group) δ 8.66 (s, 1H), 8.58 (d, J=1.8 Hz, 1H), 7.99 (s, 1H), 7.72 (s, 1H), 7.70-7.64 (m, 1H), 7.59-7.43 (m, 2H), 7.25-7.16 (m, 1H), 6.84-6.73 (m, 1H), 4.09 (s, 3H), 3.49-3.31 (m, 1H), 3.15-3.06 (m, 1H), 2.58 (s, 4H), 2.36 (s, 3H), 2.26-2.14 (m, 1H), 2.00-1.90 (m, 1H), 1.81 (dd, J=10.0, 8.1 Hz, 1H), 1.54-1.14 (m, 9H), 0.90-0.71 (m, 1H).

### Synthesis of 2-(2-azabicyclo[2.1.1]hexan-1-yl)-3-(3,5-difluoro-4-methoxyphenyl)-8-(5,6-dimethylpyridin-3-yl)-4-oxo-3,4-dihydroquinazoline-6-carbonitrile (25www)

Trifluoroacetic acid (0.42 mL, 5.50 mmol) was added to a solution of 28g (110 mg, 0.18 mmol) in DCM (10 mL) at 0° C., then stirred at room temperature overnight, then the solvent was diluted with DCM (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 3% MeOH/DCM) to afford 25www (50 mg, 55%) as a white solid. ¹H NMR (400 MHz, Chloroform-d) δ 8.64 (s, 1H), 8.58 (d, J=1.6 Hz, 1H), 8.00 (d, J=1.6 Hz, 1H), 7.73 (s, 1H), 7.02 (d, J=7.9 Hz, 2H), 4.10 (s, 3H), 2.85 (s, 2H), 2.64-2.51 (m, 4H), 2.37 (s, 3H), 1.81 (s, 2H), 1.71 (s, 1H), 1.08-0.97 (m, 2H). MS (ESI) m/z: 500.0 (M+1)⁺.

### Example 177. Synthesis of 8-(6-acetylpyridin-3-yl)-3-(3,5-difluoro-4-methoxyphenyl)-4-oxo-2-(pyrrolidin-2-yl)-3,4-dihydroquinazoline-6-carbonitrile (25xxx)

Prepared as shown in Scheme 8d.

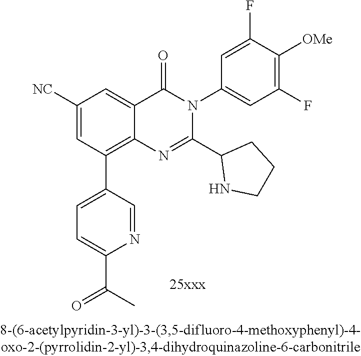 [SMILES: COc1c(F)cc(-n2c(C3CCCN3)nc3c(-c4ccc(C(C)=O)nc4)cc(C#N)cc3c2=O)cc1F]

### Synthesis of tert-butyl 2-(8-(6-acetylpyridin-3-yl)-6-cyano-3-(3,5-difluoro-4-methoxyphenyl)-4-oxo-3,4-dihydroquinolin-2-yl)pyrrolidine-1-carboxylate (28 h)

The procedure leading to 28a was followed using 22m (400 mg, 0.71 mmol), cesium carbonate (465 mg, 1.43 mmol), 1-(5-(4,4,5,5-tetramethyl-1,3,2-dioxaborolan-2-yl)pyridin-2-yl)ethan-1-one (229 mg, 0.93 mmol) and tetrakis(tri-phenylphosphine) palladium (0) (62 mg, 0.0535 mmol), 1,4-dioxane (10 mL), and H₂O (2 mL). Purification by flash chromatography (0% to 20% A (DCM:ethyl acetate=1:4)/Hexane) and a palladium scavenger (SiliaMetS Thiol, 3×120 mg) yielded 28 h (358 mg, 83%) as a yellow solid. ¹H NMR (400 MHz, Chloroform-d, a mixture of rotamers caused by -Boc group) δ 8.92-8.81 (m, 1H), 8.69-8.61 (m, 1H), 8.21-8.00 (m, 3H), 7.26-7.19 (m, 1H), 6.99-6.81 (m, 1H), 4.58-4.44 (m, 1H), 4.17-4.06 (m, 3H), 3.44-3.27 (m, 2H), 2.83-2.74 (m, 3H), 2.07-1.76 (m, 4H), 1.38-1.23 (m, 9H).

### Synthesis of 8-(6-acetylpyridin-3-yl)-3-(3,5-difluoro-4-methoxyphenyl)-4-oxo-2-(pyrrolidin-2-yl)-3,4-dihydroquinazoline-6-carbonitrile (25xxx)

Trifluoroacetic acid (0.58 mL, 7.48 mmol) was added to a solution of 28 h (150 mg, 0.25 mmol) in DCM (10 mL) at 0° C., then stirred at room temperature overnight, then the solvent was diluted with DCM (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 2% MeOH/DCM) to afford 25xxx (100 mg, 80%) as a yellow solid. ¹H NMR (400 MHz, Chloroform-d) δ 8.89 (dd, J=2.2, 0.7 Hz, 1H), 8.65 (d, J=2.0 Hz, 1H), 8.22-8.15 (m, 1H), 8.12-8.06 (m, 1H), 8.04 (d, J=2.0 Hz, 1H), 6.91 (ddt, J=29.8, 10.3, 2.3 Hz, 2H), 4.12 (s, 3H), 3.96-3.82 (m, 1H), 3.06-2.98 (m, 1H), 2.80 (s, 3H), 2.79-2.70 (m, 1H), 1.88-1.67 (m, 5H). MS (ESI) m/z: 502.1 (M+1)⁺.

### Example 178. Synthesis of 2-(2-azabicyclo[2.1.1]hexan-1-yl)-3-(3-fluoro-4-methoxyphenyl)-8-(6-methylpyridin-3-yl)-4-oxo-3,4-dihydroquinazoline-6-carbonitrile (25yyy)

Prepared as shown in Scheme 8d.

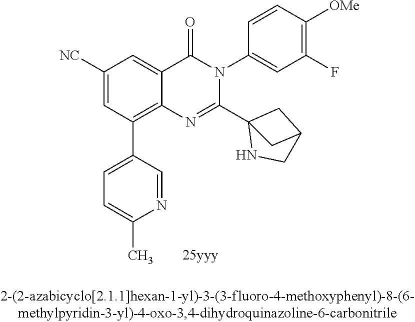 [SMILES: COc1ccc(-n2c(C34CC(CN3)C4)nc3c(-c4ccc(C)nc4)cc(C#N)cc3c2=O)cc1F]

### Synthesis of tert-butyl 1-(8-bromo-6-cyano-3-(3-fluoro-4-methoxyphenyl)-4-oxo-3,4-dihydroquinazolin-2-yl)-2-azabicyclo[2.1.1]hexane-2-carboxylate (22n)

The procedure leading to 221 was followed using 2-amino-3-bromo-5-cyanobenzoic acid (2.48 g, 0.010 mol), 2-(tert-butoxycarbonyl)-2-azabicyclo[2.1.1]hexane-1-carboxylic acid (3.5 g, 0.015 mmol), 1-methylimidazole (9.9 mL, 0.12 mol), MsCl (1.2 mL, 0.015 mol), 3-fluoro-4-methoxyaniline (3.77 g, 0.0.39 mmol), triethylamine (13 mL, 0.093 mol), TMSCl (9.2 mL, 0.072 mol), and anhydrous DCM (150 mL). Purification by flash chromatography (0% to 17% A (DCM:ethyl acetate=1:4)/Hexane)) yielded 22n (2.4 g, 41%) as a pale yellow solid. ¹H NMR (400 MHz, Chloroform-d, a mixture of rotamers caused by -Boc group) δ 8.57-8.47 (m, 1H), 8.27-8.13 (m, 1H), 7.35 (s, 1H), 7.09-6.95 (m, 1H), 6.96-6.82 (m, 1H), 4.00-3.91 (m, 3H), 3.42 (s, 1H), 3.14-3.06 (m, 1H), 2.66-2.57 (m, 1H), 2.41 (s, 1H), 2.05-1.91 (m, 2H), 1.50-1.08 (m, 9H), 0.87-0.68 (m, 1H).

### Synthesis of tert-butyl 1-(6-cyano-3-(3-fluoro-4-methoxyphenyl)-8-(6-methylpyridin-3-yl)-4-oxo-3,4-dihydroquinazolin-2-yl)-2-azabicyclo[2.1.1]hexane-2-carboxylate (28i)

The procedure of 28a was followed using 22n (300 mg, 0.53 mmol), cesium carbonate (350 mg, 1.06 mmol), (6-methylpyridin-3-yl)boronic acid (94 mg, 0.69 mmol) and tetrakis(tri-phenylphosphine) palladium (0) (46 mg, 0.04 mmol), 1,4-dioxane (8 mL), and H₂O (2 mL). Purification by flash chromatography (0% to 70% A (DCM:ethyl acetate=1:4)/Hexane) and a palladium scavenger (SiliaMetS Thiol, 3×60 mg) yielded 28i (180 mg, 60%) as a white solid. ¹H NMR (400 MHz, Chloroform-d, a mixture of rotamers caused by -Boc group) δ 8.84 (s, 1H), 8.65-8.56 (m, 1H), 8.03-7.94 (m, 1H), 7.92-7.83 (m, 1H), 7.77-7.60 (m, 2H), 7.60-7.51 (m, 1H), 7.51-7.33 (m, 2H), 7.33-7.27 (m, 1H), 7.08-6.88 (m, 2H), 4.04-3.92 (m, 3H), 3.50-3.32 (m, 1H), 3.14-3.00 (m, 1H), 2.64 (s, 3H), 2.58-2.49 (m, 1H), 2.17 (s, 1H), 1.93-1.86 (m, 1H), 1.86-1.75 (m, 1H), 1.53-1.13 (m, 9H), 0.80-0.54 (m, 1H).

### Synthesis of 2-(2-azabicyclo[2.1.1]hexan-1-yl)-3-(3-fluoro-4-methoxyphenyl)-8-(6-methylpyridin-3-yl)-4-oxo-3,4-dihydroquinazoline-6-carbonitrile (25yyy)

Trifluoroacetic acid (0.61 mL, 7.93 mmol) was added to a solution of 28i (150 mg, 0.26 mmol) in DCM (10 mL) at 0° C., then stirred at room temperature overnight, then the solvent was diluted with DCM (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 2% MeOH/DCM) to afford 25yyy (63 mg, 50%) as a white solid. ¹H NMR (400 MHz, Chloroform-d) δ 8.85-8.77 (m, 1H), 8.63-8.56 (m, 1H), 7.99 (d, J=2.0 Hz, 1H), 7.89 (dd, J=8.0, 2.3 Hz, 1H), 7.30-7.26 (m, 1H), 7.19-7.05 (m, 3H), 3.97 (s, 3H), 2.83 (q, J=8.3 Hz, 2H), 2.64 (s, 3H), 2.58-2.50 (m, 1H), 1.83-1.77 (m, 2H), 1.72 (s, 1H), 1.13-0.95 (m, 2H). MS (ESI) m/z: 467.1 (M+1)⁺.

### Example 179. Synthesis of (R)-3-(3-fluoro-4-methoxyphenyl)-8-(4-morpholinopiperidin-1-yl)-4-oxo-2-(pyrrolidin-2-yl)-3,4-dihydroquinazoline-6-carbonitrile (25zzz)

Prepared as shown in Scheme 8d.

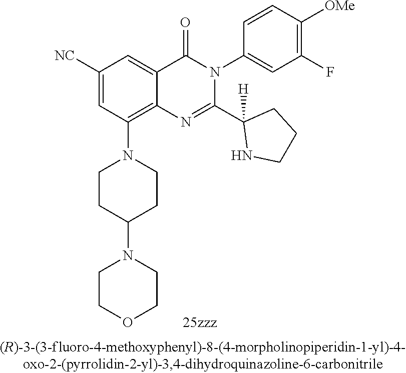 [SMILES: COc1ccc(-n2c([C@H]3CCCN3)nc3c(N4CCC(N5CCOCC5)CC4)cc(C#N)cc3c2=O)cc1F]

### Synthesis of tert-butyl (R)-2-(8-bromo-6-cyano-3-(3-fluoro-4-methoxyphenyl)-4-oxo-3,4-dihydroquinazolin-2-yl)pyrrolidine-1-carboxylate (22a)

The procedure leading to 221 was followed using 2-amino-3-bromo-5-cyanobenzoic acid (1.00 g, 4.15 mmol), Boc-2-D-proline (1.34 g, 6.22 mmol), 1-methylimidazole (1.16 mL, 14.53 mmol), MsCl (0.48 mL, 6.22 mmol), 3-fluoro-4-methoxyaniline (0.88 g, 6.22 mmol), triethylamine (5.20 mL, 37.35 mol), TMSCl (3.70 mL, 29.05 mol), and anhydrous DCM (150 mL). Purification by flash chromatography (0% to 17% A (DCM:ethyl acetate=1:4)/Hexane)) yielded 22a (1.45 g, 64%) as a pale yellow solid. ¹H NMR (400 MHz, Chloroform-d, a mixture of rotamers caused by -Boc group and atropisomers caused by —F atom) δ 8.53-8.48 (m, 1H), 8.23-8.16 (m, 1H), 7.41-7.16 (m, 1H), 7.15-7.05 (m, 1H), 6.98-6.91 (m, 1H), 4.62-4.49 (m, 1H), 4.01-3.95 (m, 3H), 3.90-3.79 (m, 1H), 3.60-3.44 (m, 1H), 2.28-1.84 (m, 4H), 1.42 (d, J=3.9 Hz, 6H), 1.29 (d, J=2.1 Hz, 3H). MS (ESI) m/z: 544.1 (M+1)⁺.

### Synthesis of tert-butyl (R)-2-(6-cyano-3-(3-fluoro-4-methoxyphenyl)-8-(4-morpholinopiperidin-1-yl)-4-oxo-3,4-dihydroquinazolin-2-yl)pyrrolidine-1-carboxylate (28j)

A mixture of 22a (520 mg, 0.96 mmol), 4-(piperidin-4-yl)morpholine (212 mg, 1.25 mmol), XPhos Pd G3 (41 mg, 0.048 mmol) and Cs₂CO₃ (947 mg, 2.88 mmol) in anhydrous toluene (15 mL) was heated to reflux for 16 h under nitrogen atmosphere. After cooling to room temperature, the mixture was concentrated to remove toluene, and diluted with H₂O (50 mL) and extracted with DCM (3×60 mL). The combined organic layers were washed with brine (150 mL), dried over anhydrous Na₂SO₄, and filtered, then the solvent was evaporated under reduced pressure to afford a pale-yellow oil. The crude product was purified with flash chromatography (0-1.5% MeOH/DCM) to give the product (200 mg), which was treated with a palladium scavenger (SiliaMetS Thiol, 70 mg), stirred at 40° C. for 8 h and at rt for 16 h, and filtered through Celite pad to remove any residual metal from the coupling reaction. Repeating this procedure of removing Pd for three times. After concentration, 28j (200 mg, 33%) was obtained as a yellow solid. ¹H NMR (400 MHz, Chloroform-d, a mixture of rotamers caused by -Boc group and atropisomers caused by —F atom) δ 8.28-8.09 (m, 1H), 7.45-7.27 (m, 1H), 7.24-7.01 (m, 2H), 7.01-6.88 (m, 1H), 4.59-4.45 (m, 1H), 4.40-4.22 (m, 1H), 4.03-3.92 (m, 3H), 3.83-3.73 (m, 4H), 3.72-3.61 (m, 1H), 3.57-3.46 (m, 1H), 2.99-2.82 (m, 1H), 2.71-2.54 (m, 4H), 2.44-2.27 (m, 1H), 2.28-1.47 (m, 10H), 1.49-1.23 (m, 9H).

### Synthesis of (R)-3-(3-fluoro-4-methoxyphenyl)-8-(4-morpholinopiperidin-1-yl)-4-oxo-2-(pyrrolidin-2-yl)-3,4-dihydroquinazoline-6-carbonitrile (25zzz)

Trifluoroacetic acid (0.73 mL, 9.48 mmol) was added to a solution of 28j (200 mg, 0.32 mmol) in DCM (10 mL) at 0° C., then stirred at room temperature overnight, then the solvent was diluted with DCM (100 mL) and washed with saturated solutions of Na₂CO₃ (3×100 mL) and brine (100 mL), dried over anhydrous Na₂SO₄, and filtered, then the solvent was evaporated under reduced pressure. The residue was purified by flash chromatography (0% to 2% MeOH/DCM) to afford 25zzz (85 mg, 51%) as a yellow solid. ¹H NMR (400 MHz, Chloroform-d, a mixture of two atropisomers (≈1:1 ratio) caused by —F atom) δ 8.14 (s, 1H), 7.33 (s, 1H), 7.14-6.97 (m, 3H), 4.03-3.85 (m, 6H), 3.77 (s, 4H), 3.27-3.16 (m, 1H), 2.90-2.76 (m, 3H), 2.63 (s, 4H), 2.44-2.34 (m, 1H), 2.10-2.02 (m, 2H), 1.86-1.74 (m, 6H). MS (ESI) m/z: 533.0 (M+1)⁺.

### Example 180. Synthesis of 3-(3,5-difluoro-4-methoxyphenyl)-8-(6-(hydroxymethyl)pyridin-3-yl)-4-oxo-2-(pyrrolidin-2-yl)-3,4-dihydroquinazoline-6-carbonitrile (25aaaa)

Prepared as shown in Scheme 8d.

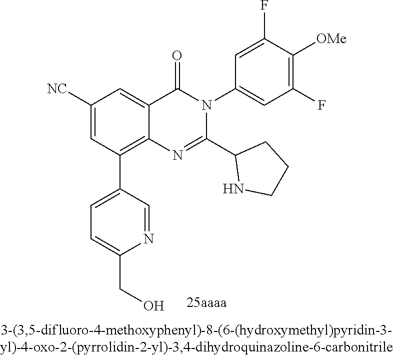 [SMILES: COc1c(F)cc(-n2c(C3CCCN3)nc3c(-c4ccc(CO)nc4)cc(C#N)cc3c2=O)cc1F]

### Synthesis of tert-butyl 2-(6-cyano-3-(3,5-difluoro-4-methoxyphenyl)-8-(6-formylpyridin-3-yl)-4-oxo-3,4-dihydroquinazolin-2-yl)pyrrolidine-1-carboxylate (28k)

A mixture of 221 (700 mg, 1.25 mmol), cesium carbonate (813 mg, 2.49 mmol), 5-(4,4,5,5-tetramethyl-1,3,2-dioxaborolan-2-yl)picolinaldehyde (378 mg, 1.62 mmol) and tetrakis(tri-phenylphosphine) palladium (0) (108 mg, 0.094 mmol) in 1,4-dioxane (20 mL) and H₂O (4 mL) was degassed and flushed with nitrogen. The mixture was heated at 85° C. for 16 h under nitrogen atmosphere. After cooling to room temperature, the mixture was concentrated to remove dioxane, and diluted with H₂O (100 mL) and extracted with DCM (3×100 mL). The combined organic layers were washed with brine (200 mL), dried over anhydrous Na₂SO₄, and filtered, then the solvent was evaporated under reduced pressure to afford a pale-yellow oil. The crude product was purified with flash chromatography (0% to 25% A (DCM:ethyl acetate=1:4)/Hexane) to give the product, which was treated with a palladium scavenger (SiliaMetS Thiol, 80 mg), stirred at 40° C. for 8 h and at rt for 16 h, and filtered through Celite pad to remove any residual metal from the coupling reaction. Repeating this procedure of removing Pd for three times. After concentration, 28k (230 mg, 31%) was obtained as a yellow solid. ¹H NMR (400 MHz, Chloroform-d, a mixture of rotamers caused by -Boc group) δ 10.22-10.15 (m, 1H), 9.03-8.92 (m, 1H), 8.70-8.61 (m, 1H), 8.23-8.03 (m, 3H), 7.26-7.18 (m, 1H), 6.98-6.81 (m, 1H), 4.57-4.44 (m, 1H), 4.17-4.07 (m, 3H), 3.47-3.28 (m, 2H), 2.12-1.97 (m, 1H), 1.92-1.76 (m, 3H), 1.29-1.23 (m, 9H).

### Synthesis of 3-(3,5-difluoro-4-methoxyphenyl)-8-(6-(hydroxymethyl)pyridin-3-yl)-4-oxo-2-(pyrrolidin-2-yl)-3,4-dihydroquinazoline-6-carbonitrile (25aaaa)

To the mixture of 28k (350 mg, 0.60 mmol) in MeOH (10 mL) cooled to 0° C. was added NaBH₄ (68 mg, 1.80 mmol). The reaction mixture was stirred at 0° C. for 1 h. Then, the reaction mixture was quenched by saturated aqueous NH₄Cl (50 mL) and concentrated to remove MeOH. The resulting residual was extracted with DCM (3×50 mL). The combined organic layers washed with brine (200 mL), dried over anhydrous Na₂SO₄, and filtered, then the solvent was evaporated under reduced pressure to afford the crude product of 29a (190 mg) as a white solid. Then, trifluoroacetic acid (0.74 mL, 9.67 mmol) was added to a solution of 29a (190 mg, 0.32 mol) in CH₂Cl₂ (10 mL) at 0° C., then stirred at room temperature overnight, then the solvent was diluted with CH₂Cl₂ (100 mL) and washed with saturated solutions of Na₂CO₃ (3×50 mL) and brine (150 mL), dried over anhydrous Na₂SO₄, and filtered, then the solvent was evaporated under reduced pressure. The residue was purified by flash chromatography (0% to 6% MeOH/CH₂Cl₂) to afford 25aaaa (108 mg, 37%) as a yellow solid. ¹H NMR (400 MHz, Chloroform-d) δ 8.81 (s, 1H), 8.62 (s, 1H), 8.01 (s, 1H), 7.94 (dd, J=8.0, 1.6 Hz, 1H), 7.42 (d, J=8.1 Hz, 1H), 6.96 (d, J=10.3 Hz, 1H), 6.88 (d, J=10.1 Hz, 1H), 4.88 (s, 2H), 4.12 (s, 3H), 4.00-3.92 (m, 1H), 3.14-3.03 (m, 1H), 2.88-2.77 (m, 1H), 1.91-1.67 (m, 4H). MS (ESI) m/z: 490.0 (M+1)⁺.

### Example 181. Synthesis of 3-(3-fluoro-4-methoxyphenyl)-8-(6-(hydroxymethyl)pyridin-3-yl)-4-oxo-2-(pyrrolidin-2-yl)-3,4-dihydroquinazoline-6-carbonitrile (25bbbb)

Prepared as shown in Scheme 8d.

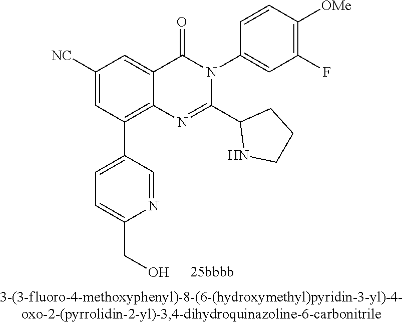 [SMILES: COc1ccc(-n2c(C3CCCN3)nc3c(-c4ccc(CO)nc4)cc(C#N)cc3c2=O)cc1F]

### Synthesis of tert-butyl 2-(8-bromo-6-cyano-3-(3-fluoro-4-methoxyphenyl)-4-oxo-3,4-dihydroquinazolin-2-yl)pyrrolidine-1-carboxylate (22d)

The procedure of 221 was followed using 2-amino-3-bromo-5-cyanobenzoic acid (1.00 g, 4.15 mmol), 2-Boc-DL-proline (1.34 g, 6.22 mmol), 1-methylimidazole (1.16 mL, 14.53 mmol), MsCl (0.48 mL, 6.22 mmol), 3-fluoro-4-methoxyaniline (0.88 g, 6.22 mmol), triethylamine (5.20 mL, 37.35 mol), TMSCl (3.70 mL, 29.05 mol), and anhydrous DCM (150 mL). Purification by flash chromatography (0% to 17% A (DCM:ethyl acetate=1:4)/Hexane)) yielded 22d (1.45 g, 64%) as a pale yellow solid. ¹H NMR (400 MHz, Chloroform-d, a mixture of rotamers caused by -Boc group and atropisomers caused by —F atom) δ 8.53-8.48 (m, 1H), 8.23-8.16 (m, 1H), 7.41-7.16 (m, 1H), 7.15-7.05 (m, 1H), 6.98-6.91 (m, 1H), 4.62-4.49 (m, 1H), 4.01-3.95 (m, 3H), 3.90-3.79 (m, 1H), 3.60-3.44 (m, 1H), 2.28-1.84 (m, 4H), 1.42 (d, J=3.9 Hz, 6H), 1.29 (d, J=2.1 Hz, 3H). MS (ESI) m/z: 544.1 (M+1)⁺.

### Synthesis of tert-butyl 2-(6-cyano-3-(3-fluoro-4-methoxyphenyl)-8-(6-formylpyridin-3-yl)-4-oxo-3,4-dihydroquinolin-2-yl)pyrrolidine-1-carboxylate (28l)

A mixture of 22d (630 mg, 1.16 mmol), cesium carbonate (756 mg, 2.32 mmol), 5-(4,4,5,5-tetramethyl-1,3,2-dioxaborolan-2-yl)picolinaldehyde (352 mg, 1.51 mmol) and tetrakis(tri-phenylphosphine) palladium (0) (201 mg, 0.17 mmol) in 1,4-dioxane (20 mL) and H₂O (4 mL) was degassed and flushed with nitrogen. The mixture was heated at 85° C. for 16 h under nitrogen atmosphere. After cooling to room temperature, the mixture was concentrated to remove dioxane, and diluted with H₂O (100 mL) and extracted with DCM (3×100 mL). The combined organic layers were washed with brine (200 mL), dried over anhydrous Na₂SO₄, and filtered, then the solvent was evaporated under reduced pressure to afford a pale-yellow oil. The crude product was purified with flash chromatography (0% to 40% A (DCM:ethyl acetate=1:4)/Hexane) to give the product, which was treated with a palladium scavenger (SiliaMetS Thiol, 120 mg), stirred at 40° C. for 8 h and at rt for 16 h, and filtered through Celite pad to remove any residual metal from the coupling reaction. Repeating this procedure of removing Pd for three times. After concentration, 281 (320 mg, 48%) was obtained as a yellow solid. ¹H NMR (400 MHz, Chloroform-d, a mixture of rotamers caused by -Boc group and atropisomers caused by —F atom) δ 10.21-10.14 (m, 1H), 9.05-8.94 (m, 1H), 8.71-8.62 (m, 1H), 8.27-8.16 (m, 1H), 8.15-8.02 (m, 2H), 7.41-7.30 (m, 1H), 7.20-7.09 (m, 1H), 7.03-6.92 (m, 1H), 4.62-4.42 (m, 1H), 4.06-3.92 (m, 3H), 3.46-3.29 (m, 2H), 2.02-1.73 (m, 4H), 1.38-1.27 (m, 9H).

### Synthesis of tert-butyl 2-(6-cyano-3-(3,5-difluoro-4-methoxyphenyl)-8-(6-(2-hydroxypropan-2-yl)pyridin-3-yl)-4-oxo-3,4-dihydroquinazolin-2-yl)pyrrolidine-1-carboxylate (25bbbb)

To the mixture of 28l (320 mg, 0.56 mmol) in MeOH (10 mL) cooled to 0° C. was added NaBH₄ (43 mg, 1.12 mmol). The reaction mixture was stirred at 0° C. for 1 h. Then, the reaction mixture was quenched by saturated aqueous NH₄Cl (50 mL) and concentrated to remove MeOH. The resulting residual was extracted with DCM (3×50 mL). The combined organic layers washed with brine (200 mL), dried over anhydrous Na₂SO₄, and filtered, then the solvent was evaporated under reduced pressure to afford the crude product of 29b (290 mg) as a white solid. Then, trifluoroacetic acid (1.13 mL, 14.7 mmol) was added to a solution of 29b (280 mg, 0.49 mol) in CH₂Cl₂ (10 mL) at 0° C., then stirred at room temperature overnight, then the solvent was diluted with CH₂Cl₂ (100 mL) and washed with saturated solutions of Na₂CO₃ (4×50 mL) and brine (150 mL), dried over anhydrous Na₂SO₄, and filtered, then the solvent was evaporated under reduced pressure. The residue was purified by flash chromatography (0% to 6% MeOH/CH₂Cl₂) to afford 25bbbb (63 mg, 24%) as a yellow solid. ¹H NMR (400 MHz, Chloroform-d, a mixture of two atropisomers (≈1:1 ratio) caused by —F atom) δ 8.81 (s, 1H), 8.63 (s, 1H), 8.00 (s, 1H), 7.97 (d, J=8.2 Hz, 1H), 7.42 (d, J=8.1 Hz, 1H), 7.20-6.97 (m, 3H), 4.88 (s, 2H), 3.98 (s, 3H), 3.93-3.81 (m, 1H), 3.14-2.99 (m, 1H), 2.81-2.67 (m, 1H), 1.91-1.71 (m, 4H). MS (ESI) m/z: 472.1 (M+1)⁺.

### Example 182. Synthesis of 3-(3,5-difluoro-4-methoxyphenyl)-8-(6-(2-hydroxypropan-2-yl)pyridin-3-yl)-4-oxo-2-(pyrrolidin-2-yl)-3,4-dihydroquinazoline-6-carbonitrile (25cccc)

Prepared as shown in Scheme 8d.

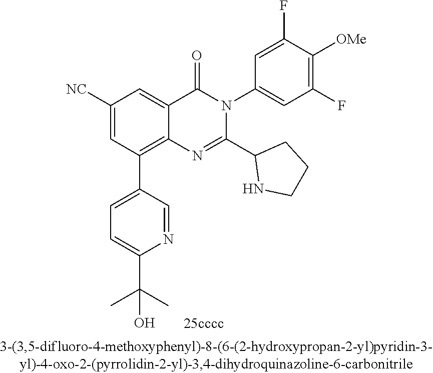 [SMILES: COc1c(F)cc(-n2c(C3CCCN3)nc3c(-c4ccc(C(C)(C)O)nc4)cc(C#N)cc3c2=O)cc1F]

### Synthesis of tert-butyl 2-(6-cyano-3-(3,5-difluoro-4-methoxyphenyl)-8-(6-(2-hydroxypropan-2-yl)pyridin-3-yl)-4-oxo-3,4-dihydroquinazolin-2-yl)pyrrolidine-1-carboxylate (29c)

To the mixture of 28h (200 mg, 0.33 mmol) in anhydrous THF (10 mL) cooled to 0° C. was added a solution of CH₃MgBr in ether (0.33 mL, 3.0N, 1.00 mmol). The reaction mixture was stirred at 0° C. for 3 h. Then, the reaction mixture was quenched by saturated aqueous NH₄Cl (50 mL) and extracted with DCM (3×50 mL). The combined organic layers washed with brine (200 mL), dried over anhydrous Na₂SO₄, and filtered, then the solvent was evaporated under reduced pressure to give the crude product. It was purified with flash chromatography (0% to 60% A (DCM:ethyl acetate=1:4)/Hexane) to deliver the product to afford 29c (70 mg, 34%) as a white solid. ¹H NMR (400 MHz, Chloroform-d) δ 8.79-8.62 (m, 1H), 8.62-8.58 (m, 1H), 8.08-7.92 (m, 2H), 7.57-7.44 (m, 1H), 7.26-7.20 (m, 1H), 6.99-6.81 (m, 1H), 5.01-4.92 (m, 1H), 4.54-4.43 (m, 1H), 4.16-4.07 (m, 3H), 3.45-3.24 (m, 2H), 2.03-1.76 (m, 4H), 1.69-1.58 (m, 6H), 1.41-1.28 (m, 9H).

### Synthesis of 3-(3,5-difluoro-4-methoxyphenyl)-8-(6-(2-hydroxypropan-2-yl)pyridin-3-yl)-4-oxo-2-(pyrrolidin-2-yl)-3,4-dihydroquinazoline-6-carbonitrile (25cccc)

Trifluoroacetic acid (0.37 mL, 4.86 mmol) was added to a solution of pale yellow solid 29c (100 mg, 0.16 mmol) in DCM (8 mL) at 0° C., then stirred at room temperature overnight, then the solvent was diluted with DCM (50 mL) and washed with saturated solutions of Na₂CO₃ (4×50 mL) and brine (50 mL), dried over anhydrous Na₂SO₄, and filtered, then the solvent was evaporated under reduced pressure. The residue was purified by flash chromatography (0% to 20% MeOH/DCM) to afford 25cccc (68 mg, 81%) as a yellow solid. ¹H NMR (400 MHz, Chloroform-d) δ 8.79-8.72 (m, 1H), 8.62 (d, J=2.0 Hz, 1H), 8.02 (d, J=2.0 Hz, 1H), 7.96 (dd, J=8.2, 2.3 Hz, 1H), 7.59-7.48 (m, 1H), 6.99-6.92 (m, 1H), 6.92-6.84 (m, 1H), 4.80 (s, 1H), 4.12 (s, 3H), 3.94-3.86 (m, 1H), 3.06-2.97 (m, 1H), 2.82-2.72 (m, 1H), 1.85-1.74 (m, 4H), 1.63 (s, 6H). MS (ESI) m/z: 518.2 (M+1)⁺.

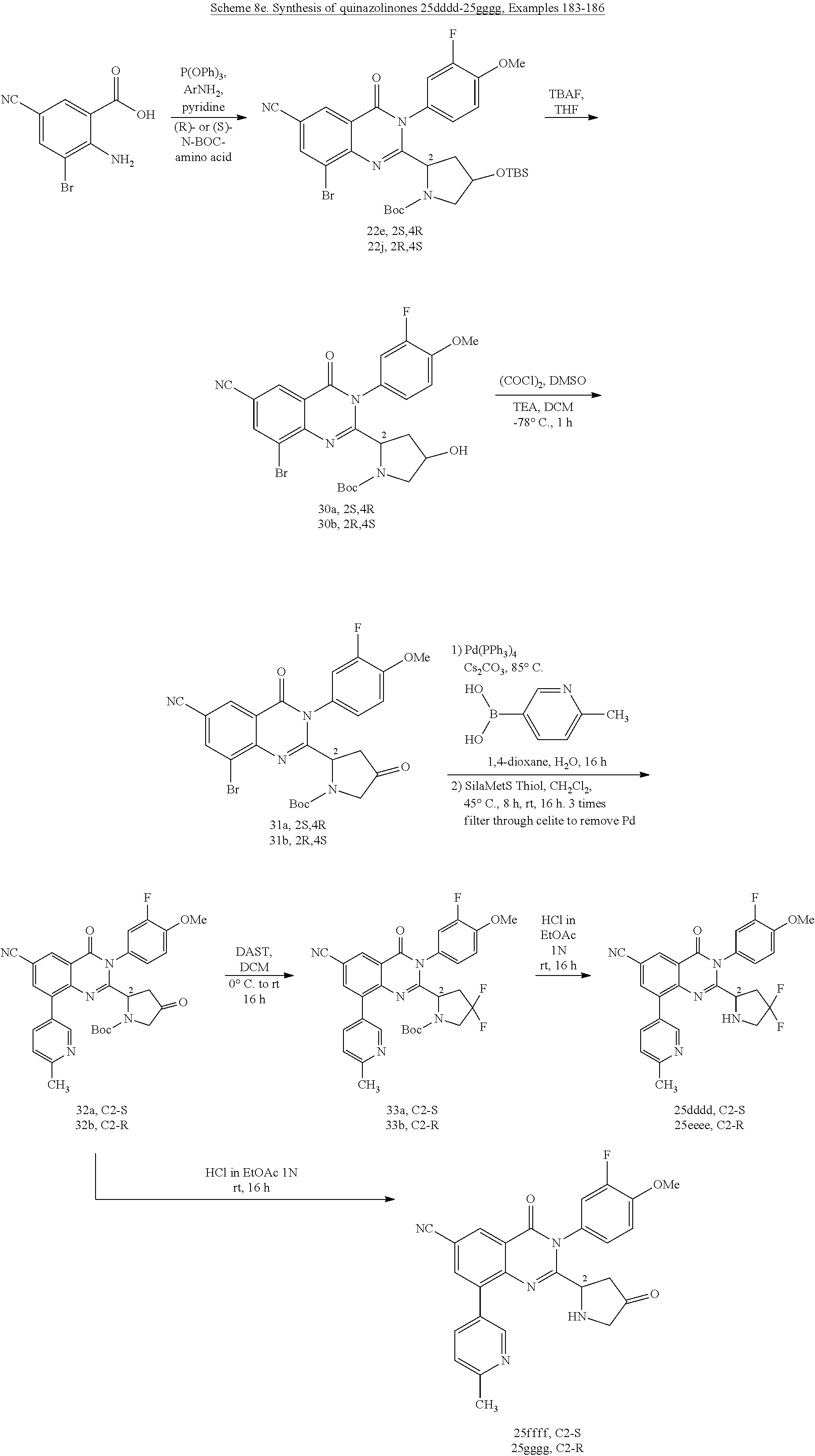

### Example 183. (S)-2-(4,4-difluoropyrrolidin-2-yl)-3-(3-fluoro-4-methoxyphenyl)-8-(6-methylpyridin-3-yl)-4-oxo-3,4-dihydroquinazoline-6-carbonitrile (25dddd)

Prepared as shown in Scheme 8e.

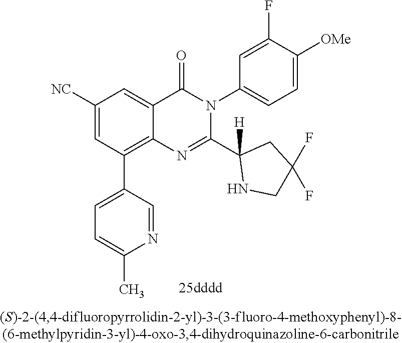 [SMILES: COc1ccc(-n2c([C@@H]3CC(F)(F)CN3)nc3c(-c4ccc(C)nc4)cc(C#N)cc3c2=O)cc1F]

### Synthesis of tert-butyl (2S,4R)-2-(8-bromo-6-cyano-3-(3-fluoro-4-methoxyphenyl)-4-oxo-3,4-dihydroquinazolin-2-yl)-4-((tert-butyldimethylsilyl)oxy)pyrrolidine-1-carboxylate (22e)

2-amino-3-bromo-5-cyanobenzoic acid (500 mg, 2.08 mmol) and (2S,4R)-1-(tert-butoxycarbonyl)-4-((tert-butyldimethylsilyl)oxy)pyrrolidine-2-carboxylic acid (935 mg, 2.71 mmol) were dissolved in dry pyridine (8 mL). triphenyl phosphite (1.64 mL, 6.25 mmol) was added to the mixture and the reaction mixture was heated at 130° C. with MWI for 30 min. 3-Fluoro-4-methoxyaniline (353 mg, 2.50 mmol) was added to the mixture and the reaction mixture was heated at 100° C. with MWI for 30 min. Pyridine was removed in vacuo and the residue was diluted with CH₂Cl₂ (100 mL) and washed with 1N HCl aqueous (100 mL). The organic layer was washed with H₂O (50 mL) and brine (50 mL), dried over anhydrous Na₂SO₄, and filtered, then the solvent was evaporated under reduced pressure. The residue was purified by flash chromatography (0% to 13% A (DCM:ethyl acetate=1:4)/Hexane)) yielded 22e (500 mg, 71%) as yellow solid. ¹H NMR (400 MHz, Chloroform-d, a mixture of rotamers caused by -Boc group and atropisomers caused by —F atom) δ 8.80-8.63 (m, 2H), 8.14-7.93 (m, 2H), 7.52-7.34 (m, 2H), 7.32-7.12 (m, 2H), 7.05-6.92 (m, 1H), 4.70-4.49 (m, 1H), 4.48-4.29 (m, 1H), 4.11-3.95 (m, 3H), 3.68-3.53 (m, 1H), 3.39-3.21 (m, 1H), 2.78-2.62 (m, 3H), 2.14-1.90 (m, 2H), 1.44-1.43 (m, 4H), 1.36-1.32 (m, 5H), 0.86-0.73 (m, 9H), 0.07-−0.05 (m, 6H).

### Synthesis of tert-butyl (2S,4R)-2-(8-bromo-6-cyano-3-(3-fluoro-4-methoxyphenyl)-4-oxo-3,4-dihydroquinazolin-2-yl)-4-hydroxypyrrolidine-1-carboxylate (30a)

To the mixture of 22e (700 mg, 1.04 mmol) in THE (10 mL) was added dropwise the solution of TBAF in water (0.76 mL, w %=75%, 2.08 mmol) at 0° C. The reaction mixture was stirred at rt for 3 h. It was extracted with DCM (3×100 mL). The combined organic layers were washed with brine (300 mL), dried over anhydrous Na₂SO₄, and filtered, then the solvent was evaporated under reduced pressure to afford a pale-yellow oil. The crude product was purified with flash chromatography (0% to 40% A (DCM:ethyl acetate=1:4)/Hexane) to afford 30a (330 mg, 57%) as a white solid. ¹H NMR (400 MHz, Chloroform-d, a mixture of rotamers caused by -Boc group and atropisomers caused by —F atom) δ 8.56-8.46 (m, 1H), 8.26-8.14 (m, 1H), 7.52-7.33 (m, 1H), 7.19-7.06 (m, 1H), 6.96-6.86 (m, 1H), 4.72-4.48 (m, 2H), 4.00-3.89 (m, 4H), 3.68-3.50 (m, 1H), 2.27-2.06 (m, 2H), 1.43-1.25 (m, 9H).

### Synthesis of tert-butyl (S)-2-(8-bromo-6-cyano-3-(3-fluoro-4-methoxyphenyl)-4-oxo-3,4-dihydroquinazolin-2-yl)-4-oxopyrrolidine-1-carboxylate (31a)

To the mixture of oxalyl chloride (61 μL, 0.72 mmol) in DCM (10 mL) under N₂ cooled to −78° C. was added dropwise DMSO (103 μL, 1.44 mmol). After being stirred at −78° C. for 30 mins, a solution of 30a (310 mg, 0.55 mmol) in DCM (5 mL) was added into the reaction mixture and stirred at −78° C. for 30 mins. Then, triethylamine (0.16 mL, 1.17 mmol) was added into the reaction mixture, and stirred at −78° C. for 1 h. It was quenched by water (50 mL) and extracted with DCM (3×100 mL). The combined organic layers were washed with brine (300 mL), dried over anhydrous Na₂SO₄, and filtered, then the solvent was evaporated under reduced pressure to afford 31a (290 mg, 94%) as a yellow oil. ¹H NMR (400 MHz, Chloroform-d, a mixture of rotamers caused by -Boc group and atropisomers caused by —F atom) δ 8.56-8.43 (m, 1H), 8.25-8.13 (m, 1H), 7.49-7.31 (m, 1H), 7.23-7.09 (m, 1H), 6.98-6.84 (m, 1H), 5.06-4.94 (m, 1H), 4.47-4.33 (m, 1H), 4.04-3.85 (m, 4H), 2.80-2.62 (m, 1H), 2.59-2.41 (m, 1H), 1.49-1.35 (m, 9H).

### Synthesis of tert-butyl (S)-2-(6-cyano-3-(3-fluoro-4-methoxyphenyl)-8-(6-methylpyridin-3-yl)-4-oxo-3,4-dihydroquinazolin-2-yl)-4-oxopyrrolidine-1-carboxylate (32a)

A mixture of 31a (310 mg, 0.56 mmol), cesium carbonate (363 mg, 1.11 mmol), 2-methylpyridine-5-boronic acid (84 mg, 0.61 mmol), tetrakis(tri-phenylphosphine) palladium (0) (13 mg, 0.011 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 65% A (DCM:ethyl acetate=1:4)/Hexane) to give the product, which was treated with a palladium scavenger (SiliaMetS Thiol, 50 mg), stirred at 40° C. for 8 h and at rt for 16 h, and filtered through Celite pad to remove any residual metal from the coupling reaction. Repeating this procedure of removing Pd for three times (Adding new palladium scavenger and being stirred for 24 h for three times). After concentration, 32a (150 mg, 47%) was obtained as a yellow solid. ¹H NMR (400 MHz, Chloroform-d, a mixture of rotamers caused by -Boc group and atropisomers caused by —F atom) δ 8.72-8.59 (m, 1H), 8.30-7.91 (m, 2H), 7.70-7.58 (m, 1H), 7.54-7.38 (m, 2H), 7.23-6.93 (m, 2H), 4.98 (t, J=10.6 Hz, 1H), 4.05-3.87 (m, 3H), 3.83-3.49 (m, 2H), 3.10-2.78 (m, 3H), 2.78-2.48 (m, 2H), 1.38-1.22 (m, 9H).

### Synthesis of (S)-2-(4,4-difluoropyrrolidin-2-yl)-3-(3-fluoro-4-methoxyphenyl)-8-(6-methylpyridin-3-yl)-4-oxo-3,4-dihydroquinazoline-6-carbonitrile (25dddd)

To the mixture of 32a (70 mg, 0.12 mmol) in DCM (2 mL) was added dropwise (diethylamino)sulfur trifluoride (81 μL, 0.62 mmol) at −78° C. The reaction mixture was stirred at rt for 16 h. It was quenched with water (10 mL) and extracted with DCM (3×30 mL). The combined organic layers were washed with brine (100 mL), dried over anhydrous Na₂SO₄, and filtered, then the solvent was evaporated under reduced pressure to afford the crude product of 33a (60 mg) as a yellow oil, which was directly was used in the next step. A solution of HCl in EtOAc (2 mL, 1N, 2 mmol) was added to a solution of 33a (65 mg, 0.11 mmol) in EtOAc (2 mL) at 0° C., then stirred at room temperature overnight, then the reaction mixture was then adjusted to pH 11 with saturated solutions of Na₂CO₃ and concentrated to remove EtOAc. The resulting residual was diluted with water (50 mL) and extracted with DCM (3×50 mL). The combined organic layers were washed with brine (100 mL), dried over anhydrous Na₂SO₄, and filtered, then the solvent was evaporated under reduced pressure. The residue was purified by flash chromatography (0-2% MeOH/DCM) to afford 25dddd (13 mg, 22%) as a pale-yellow solid. ¹H NMR (400 MHz, Chloroform-d, a mixture of two atropisomers (≈1:1 ratio) caused by —F atom) δ 8.72 (s, 1H), 8.61 (d, J=2.0 Hz, 1H), 8.01 (d, J=1.9 Hz, 1H), 7.86 (dd, J=8.0, 2.0 Hz, 1H), 7.31 (d, J=8.0 Hz, 1H), 7.19-7.06 (m, 2H), 7.03-6.93 (m, 1H), 4.28-4.07 (m, 1H), 3.99 (s, 3H), 3.33-3.20 (m, 1H), 2.99 (dd, J=20.0, 9.9 Hz, 1H), 2.67 (s, 3H), 2.49-2.35 (m, 1H), 2.30-2.16 (m, 1H). MS (ESI) m/z: 492.0 (M+1)⁺.

### Example 184. Synthesis of (R)-2-(4,4-difluoropyrrolidin-2-yl)-3-(3-fluoro-4-methoxyphenyl)-8-(6-methylpyridin-3-yl)-4-oxo-3,4-dihydroquinazoline-6-carbonitrile (25eeee)

Prepared as shown in Scheme 8e and as described for 25dddd but using the (R)-enantiomer of the Boc-protected amino acid.

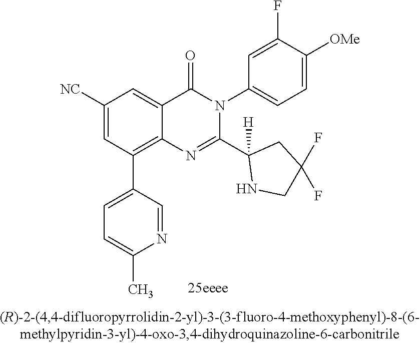 [SMILES: COc1ccc(-n2c([C@H]3CC(F)(F)CN3)nc3c(-c4ccc(C)nc4)cc(C#N)cc3c2=O)cc1F]

### Synthesis of (R)-2-(4,4-difluoropyrrolidin-2-yl)-3-(3-fluoro-4-methoxyphenyl)-8-(6-methylpyridin-3-yl)-4-oxo-3,4-dihydroquinazoline-6-carbonitrile (25eeee)

To the mixture of 32b (400 mg, 0.70 mmol) in DCM (10 mL) was added dropwise (diethylamino)sulfur trifluoride (0.45 mL, 3.51 mmol) at −78° C. The reaction mixture was stirred at rt for 16 h. It was quenched by water (10 mL), and extracted with DCM (3×50 mL). The combined organic layers were washed with brine (100 mL), dried over anhydrous Na₂SO₄, and filtered, then the solvent was evaporated under reduced pressure to afford the crude product 33b (360 mg) as a yellow oil, which was directly was used in the next step. A solution of HCl in EtOAc (15 mL, 1N, 15 mmol) was added to a solution of 33b (360 mg, 0.61 mmol) in EtOAc (5 mL) at 0° C., then stirred at room temperature overnight, then the reaction mixture was then adjusted to pH 11 with saturated solutions of Na₂CO₃ and concentrated to remove EtOAc. The resulting residual was diluted with water (50 mL) and extracted with DCM (3×50 mL). The combined organic layers were washed with brine (100 mL), dried over anhydrous Na₂SO₄, and filtered, then the solvent was evaporated under reduced pressure. The residue was purified by flash chromatography (0-2% MeOH/DCM) to afford 25eeee (100 mg, 33%) as a pale-yellow solid. ¹H NMR (400 MHz, Chloroform-d, a mixture of two atropisomers (≈1:1 ratio) caused by —F atom) δ 8.71 (d, J=2.3 Hz, 1H), 8.61 (d, J=1.9 Hz, 1H), 8.01 (d, J=1.9 Hz, 1H), 7.86 (dd, J=8.0, 2.4 Hz, 1H), 7.34-7.27 (m, 1H), 7.18-7.07 (m, 2H), 7.02-6.93 (m, 1H), 4.19 (q, J=7.9 Hz, 1H), 3.99 (s, 3H), 3.34-3.19 (m, 1H), 3.05-2.90 (m, 1H), 2.67 (s, 3H), 2.49-2.35 (m, 1H), 2.29-2.16 (m, 1H). MS (ESI) m/z: 492.0 (M+1)⁺.

### Example 185. Synthesis of (S)-3-(3-fluoro-4-methoxyphenyl)-8-(6-methylpyridin-3-yl)-4-oxo-2-(4-oxopyrrolidin-2-yl)-3,4-dihydroquinazoline-6-carbonitrile (25ffff)

Prepared as shown in Scheme 8e.

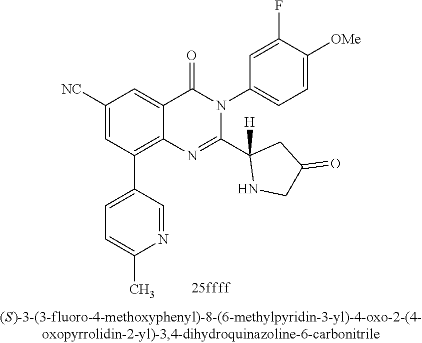 [SMILES: COc1ccc(-n2c([C@@H]3CC(=O)CN3)nc3c(-c4ccc(C)nc4)cc(C#N)cc3c2=O)cc1F]

### Synthesis of (S)-3-(3-fluoro-4-methoxyphenyl)-8-(6-methylpyridin-3-yl)-4-oxo-2-(4-oxopyrrolidin-2-yl)-3,4-dihydroquinazoline-6-carbonitrile (25ffff)

A solution of HCl in EtOAc (5 mL, 1N, 5 mmol) was added to a solution of 32a (200 mg, 0.35 mmol) in EtOAc (2 mL) at 0° C., then stirred at room temperature overnight, then the reaction mixture was then adjusted to pH 11 with saturated solutions of Na₂CO₃ and concentrated to remove EtOAc. The resulting residual was diluted with water (50 mL) and extracted with DCM (3×50 mL). The combined organic layers were washed with brine (100 mL), dried over anhydrous Na₂SO₄, and filtered, then the solvent was evaporated under reduced pressure. The residue was purified by flash chromatography (0-2% MeOH/DCM) to afford 25ffff (27 mg, 18%) as a pale-yellow solid. ¹H NMR (400 MHz, Methanol-d₄, a mixture of two atropisomers (≈1:1 ratio) caused by —F atom) δ 8.52 (d, J=2.2 Hz, 1H), 8.44 (d, J=2.0 Hz, 1H), 8.13-8.05 (m, 1H), 7.89 (dd, J=8.0, 2.3 Hz, 1H), 7.39-7.28 (m, 1H), 7.27-7.05 (m, 3H), 4.36-4.24 (m, 1H), 3.87 (s, 3H), 3.14 (dd, J=18.1, 3.2 Hz, 1H), 2.96 (d, J=18.0 Hz, 1H), 2.53 (s, 3H), 2.48-2.39 (m, 1H), 2.30-2.18 (m, 1H). MS (ESI) m/z: 470.0 (M+1)⁺.

### Example 186. Synthesis of (R)-3-(3-fluoro-4-methoxyphenyl)-8-(6-methylpyridin-3-yl)-4-oxo-2-(4-oxopyrrolidin-2-yl)-3,4-dihydroquinazoline-6-carbonitrile (25gggg)

Prepared as shown in Scheme 8e.

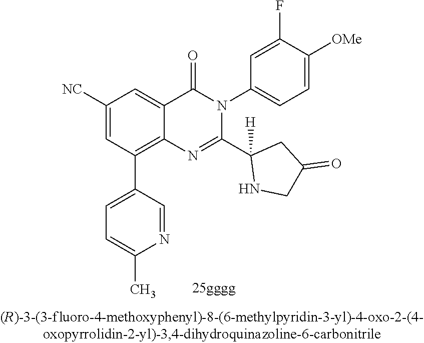 [SMILES: COc1ccc(-n2c([C@H]3CC(=O)CN3)nc3c(-c4ccc(C)nc4)cc(C#N)cc3c2=O)cc1F]

### Synthesis of tert-butyl (2R,4S)-2-(8-bromo-6-cyano-3-(3-fluoro-4-methoxyphenyl)-4-oxo-3,4-dihydroquinazolin-2-yl)-4-((tert-butyldimethylsilyl)oxy)pyrrolidine-1-carboxylate (22j)

The procedure leading to 30a was followed but using 2-amino-3-bromo-5-cyanobenzoic acid (1.00 g, 4.17 mmol), (2R,4S)-1-(tert-butoxycarbonyl)-4-((tert-butyldimethylsilyl)oxy)pyrrolidine-2-carboxylic acid (2.00 g, 5.41 mmol), triphenyl phosphite (3.50 mL, 12.51 mmol) and 3-fluoro-4-methoxyaniline (0.71 g, 5.00 mmol), and pyridine (8 mL). Purification by flash chromatography (0% to 13% A (DCM:ethyl acetate=1:4)/Hexane)) yielded 22j (1.26 g, 45%) as a pink solid. ¹H NMR (400 MHz, Chloroform-d, a mixture of rotamers caused by -Boc group and atropisomers caused by —F atom) δ 8.55-8.45 (m, 1H), 8.25-8.14 (m, 1H), 7.47-7.34 (m, 1H), 7.26-7.05 (m, 7H), 7.04-6.86 (m, 2H), 4.78-4.54 (m, 2H), 4.01-3.94 (m, 3H), 3.94-3.82 (m, 2H), 3.48-3.28 (m, 2H), 2.17-1.88 (m, 3H), 1.42-1.40 (m, 6H), 1.27-1.23 (m, 3H), 0.88 (s, 4H), 0.84-0.80 (m, 9H), 0.14-−0.06 (m, 12H).

### Synthesis of tert-butyl (2R,4S)-2-(8-bromo-6-cyano-3-(3-fluoro-4-methoxyphenyl)-4-oxo-3,4-dihydroquinazolin-2-yl)-4-hydroxypyrrolidine-1-carboxylate (30b)

The procedure leading to 30a was followed using 22j (3.1 g, 4.61 mmol), a solution of TBAF in DCM (5.53 mL, 1N, 5.53 mmol), and THF (30 mL). Purification by flash chromatography (0% to 40% A (DCM:ethyl acetate=1:4)/Hexane)) yielded 30b (1.8 g, 70%) as a yellow solid. ¹H NMR (400 MHz, Chloroform-d, a mixture of rotamers caused by -Boc group and atropisomers caused by —F atom) δ 8.56-8.44 (m, 1H), 8.27-8.15 (m, 1H), 7.50-7.33 (m, 1H), 7.21-7.06 (m, 1H), 6.96-6.85 (m, 1H), 4.71-4.61 (m, 1H), 4.60-4.32 (m, 1H), 4.03-3.89 (m, 4H), 3.71-3.48 (m, 1H), 2.30-2.05 (m, 2H), 1.44-1.37 (m, 6H), 1.33-1.19 (m, 3H).

### Synthesis of tert-butyl (R)-2-(8-bromo-6-cyano-3-(3-fluoro-4-methoxyphenyl)-4-oxo-3,4-dihydroquinazolin-2-yl)-4-oxopyrrolidine-1-carboxylate (31b)

The procedure leading to 31a was followed using oxalyl chloride (0.34 mL, 3.96 mmol), DMSO (0.56 mL, 7.93 mmol), 30b (1.7 g, 3.05 mmol), triethylamine (2.12 mL, 15.25 mmol), and DCM (30 mL) to yield 31b (1.4 g, 83%) as a yellow oil. ¹H NMR (400 MHz, Chloroform-d, a mixture of rotamers caused by -Boc group and atropisomers caused by —F atom) δ 8.50 (t, J=2.2 Hz, 1H), 8.25-8.14 (m, 1H), 7.50-7.35 (m, 1H), 7.24-7.09 (m, 1H), 6.97-6.84 (m, 1H), 5.08-4.94 (m, 1H), 4.50-4.33 (m, 1H), 4.05-3.88 (m, 4H), 2.89-2.62 (m, 1H), 2.57-2.35 (m, 1H), 1.52-1.33 (m, 9H).

### Synthesis of tert-butyl (R)-2-(6-cyano-3-(3-fluoro-4-methoxyphenyl)-8-(6-methylpyridin-3-yl)-4-oxo-3,4-dihydroquinazolin-2-yl)-4-oxopyrrolidine-1-carboxylate (32b)

The procedure leading to 32a was followed except using 31b (1.4 g, 2.52 mmol), cesium carbonate (1.64 g, 5.04 mmol), 2-methylpyridine-5-boronic acid (448 mg, 3.27 mmol), tetrakis(tri-phenylphosphine) palladium (0) (218 mg, 0.19 mmol), 1,4-dioxane (30 mL), and H₂O (5 mL). Purification by flash chromatography (0% to 60% A (DCM:ethyl acetate=1:4)/Hexane)) and a palladium scavenger (SiliaMetS Thiol, 3×250 mg) yielded 32b (750 mg, 52%) as a yellow solid. ¹H NMR (400 MHz, Chloroform-d, a mixture of rotamers caused by -Boc group and atropisomers caused by —F atom) δ 8.68-8.49 (m, 2H), 8.05-7.93 (m, 1H), 7.74-7.63 (m, 2H), 7.57-7.36 (m, 3H), 7.34-7.28 (m, 1H), 7.23-7.09 (m, 1H), 6.98-6.89 (m, 1H), 4.98 (t, J=10.0 Hz, 1H), 4.06-3.91 (m, 3H), 3.83-3.63 (m, 2H), 2.94-2.56 (m, 4H), 2.49-2.36 (m, 1H), 1.48-1.22 (m, 9H).

### Synthesis of (R)-3-(3-fluoro-4-methoxyphenyl)-8-(6-methylpyridin-3-yl)-4-oxo-2-(4-oxopyrrolidin-2-yl)-3,4-dihydroquinazoline-6-carbonitrile (25gggg)

A solution of HCl in EtOAc (5 mL, 1N, 5 mmol) was added to a solution of 32b (250 mg, 0.44 mmol) in EtOAc (2 mL) at 0° C., then stirred at room temperature overnight, then the reaction mixture was then adjusted to pH 11 with saturated solutions of Na₂CO₃ and concentrated to remove EtOAc. The resulting residual was diluted with water (50 mL) and extracted with DCM (3×50 mL). The combined organic layers were washed with brine (100 mL), dried over anhydrous Na₂SO₄, and filtered, then the solvent was evaporated under reduced pressure. The residue was purified by flash chromatography (0-2% MeOH/DCM) to afford 25gggg (120 mg, 58%) as a pale-yellow solid. ¹H NMR (400 MHz, Methanol-d₄, a mixture of two atropisomers (≈1:1 ratio) caused by —F atom) δ 8.52 (d, J=2.2 Hz, 1H), 8.44 (d, J=2.0 Hz, 1H), 8.13-8.05 (m, 1H), 7.89 (dd, J=8.0, 2.3 Hz, 1H), 7.39-7.28 (m, 1H), 7.27-7.05 (m, 3H), 4.36-4.24 (m, 1H), 3.87 (s, 3H), 3.14 (dd, J=18.1, 3.2 Hz, 1H), 2.96 (d, J=18.0 Hz, 1H), 2.53 (s, 3H), 2.48-2.39 (m, 1H), 2.30-2.18 (m, 1H). MS (ESI) m/z: 470.0 (M+1)⁺.

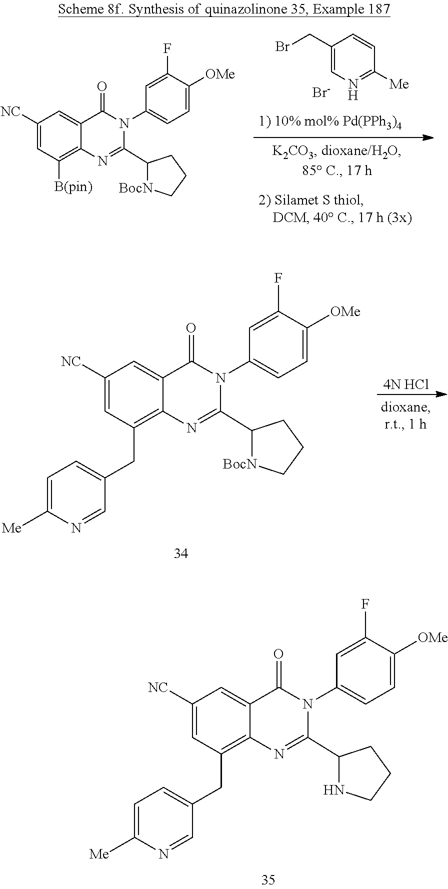

### Example 187. Synthesis of 3-(3-fluoro-4-methoxyphenyl)-8-((6-methylpyridin-3-yl)methyl)-4-oxo-2-(pyrrolidin-2-yl)-3,4-dihydroquinazoline-6-carbonitrile (35)

Prepared as shown in Scheme 8f.

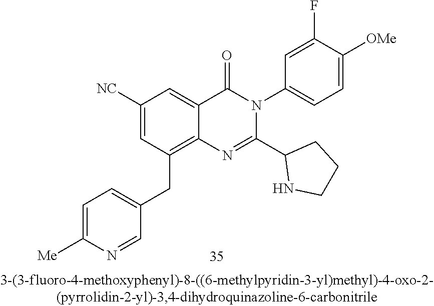 [SMILES: COc1ccc(-n2c(C3CCCN3)nc3c(Cc4ccc(C)nc4)cc(C#N)cc3c2=O)cc1F]

### Synthesis of tert-butyl (R)-2-(6-cyano-3-(3-fluoro-4-methoxyphenyl)-4-oxo-8-(4,4,5,5-tetramethyl-1,3,2-dioxaborolan-2-yl)-3,4-dihydroquinazolin-2-yl)pyrrolidine-1-carboxylate

A solution of tert-butyl (R)-2-(8-bromo-6-cyano-3-(3-fluoro-4-methoxyphenyl)-4-oxo-3,4-dihydroquinazolin-2-yl) pyrrolidine-1-carboxylate 22a (100 mg, 0.18 mmol), B₂Pin₂ (70 mg, 0.27 mmol) and KOAc (54 mg, 0.55 mmol) in dioxane (5 mL) was degassed with argon for 10 min. Pd(dppf)Cl₂-DCM (CAS: 95464-05-4) (15 mg, 0.018 mmol) was added, and the resulting reaction mixture was heated at 100° C. for 20 h. The reaction mixture was cooled to rt, diluted with EtOAc (4 mL) and brine solution (4 mL) was added. After separating the organic phase, the aqueous layer was further extracted with CH₂Cl₂ (2×3 mL). Combined organic layers were dried over Na₂SO₄ and concentrated under reduced pressure. The crude material was purified using column chromatography where the desired boronate ester intermediate (tert-butyl (R)-2-(6-cyano-3-(3-fluoro-4-methoxyphenyl)-4-oxo-8-(4,4,5,5-tetramethyl-1,3,2-dioxaborolan-2-yl)-3,4-dihydroquinazolin-2-yl)pyrrolidine-1-carboxylate) was obtained as brown colored oil (93 mg, 85%) which was carried forward to the next step.

### Synthesis of tert-butyl 2-(6-cyano-3-(3-fluoro-4-methoxyphenyl)-8-((6-methylpyridin-3-yl)methyl)-4-oxo-3,4-dihydroquinazolin-2-yl)pyrrolidine-1-carboxylate (34)

A microwave vial with stir bar was charged with 5-(bromomethyl)-2-methylpyridinium bromide (68 mg, 0.25 mmol, 1.5 equiv.), boronic ester (100 mg, 0.17 mmol, 1 equiv.), K₂CO₃ (70 mg, 0.51 mmol, 3 equiv.), and Pd(PPh₃)₄ (20 mg, 0.017 mmol, 0.1 equiv.). A 4:1 mixture of 1,4-dioxane:H₂O was added via syringe (2.5 mL). The vial was sealed with a septum and the mixture was sparged with N₂ for 15 min. After 15 min, the reaction was heated to 80° C. for 17 h. After 17 h, the reaction was cooled to room temperature and diluted with EtOAc and H₂O (10 mL each). The layers were shaken and separated. The aqueous phase was washed twice with EtOAc (10 mL each). The combined organics were dried over Na₂SO₄, filtered, and concentrated. The crude reaction mixture was purified via silica gel chromatography (3:1 to 1:1 hexanes:EtOAc). Trace palladium was removed by dissolving the purified product (34) in DCM (0.2 M) and adding SiliametS Thiol Metal Scavenger (33 wt %) to the solution. The mixture was heated to 40° C. for 8 h before being filtered through celite. This procedure was repeated two more times. tert-butyl 2-(6-cyano-3-(3-fluoro-4-methoxyphenyl)-8-((6-methylpyridin-3-yl)methyl)-4-oxo-3,4-dihydroquinazolin-2-yl)pyrrolidine-1-carboxylate (34) was obtained as a mixture of desired product and triphenylphosphine oxide (TPPO) which was used without further purification.

### Synthesis of 3-(3-fluoro-4-methoxyphenyl)-8-((6-methylpyridin-3-yl)methyl)-4-oxo-2-(pyrrolidin-2-yl)-3,4-dihydroquinazoline-6-carbonitrile (35)

Compound 34 was added to a vial with stir bar and dissolved in 4N HCl in 1,4-dioxane (1 mL). The reaction was allowed to stir at room temperature for 1 hour. The solvent was removed in vacuo and the crude residue was dissolved in DCM (5 mL). The residual HCl was quenched with sat. aq. NaHCO₃ (5 mL). The layers were shaken and separated and the aqueous phase was washed with DCM (2×, 5 mL). The combined organics were dried over Na₂SO₄, filtered, and concentrated. The crude reaction mixture was purified via silica gel chromatography (0 to 5% (4:1 MeOH:sat. aq. NH₄OH) in DCM). 3-(3-fluoro-4-methoxyphenyl)-8-((6-methylpyridin-3-yl)methyl)-4-oxo-2-(pyrrolidin-2-yl)-3,4-dihydroquinazoline-6-carbonitrile 35 was obtained as a white solid (10 mg, 0.021 mmol, 12% yield over 2 steps). ¹H NMR (400 MHz, CDCl₃) δ 8.47 (s, 1H), 8.43 (s, 1H), 7.73 (d, J=1.5 Hz, 1H), 7.39 (d, J=7.9 Hz, 1H), 7.16-6.94 (m, 4H), 4.48-4.25 (m, 2H), 3.97 (s, 3H), 3.88 (q, J=7.7, 7.0 Hz, 1H), 3.21 (dt, J=10.7, 5.3 Hz, 1H), 2.85-2.75 (m, 1H), 2.54 (s, 3H), 1.86-1.65 (m, 4H). MS (ESI) m/z: 470.2 (M+1)⁺.

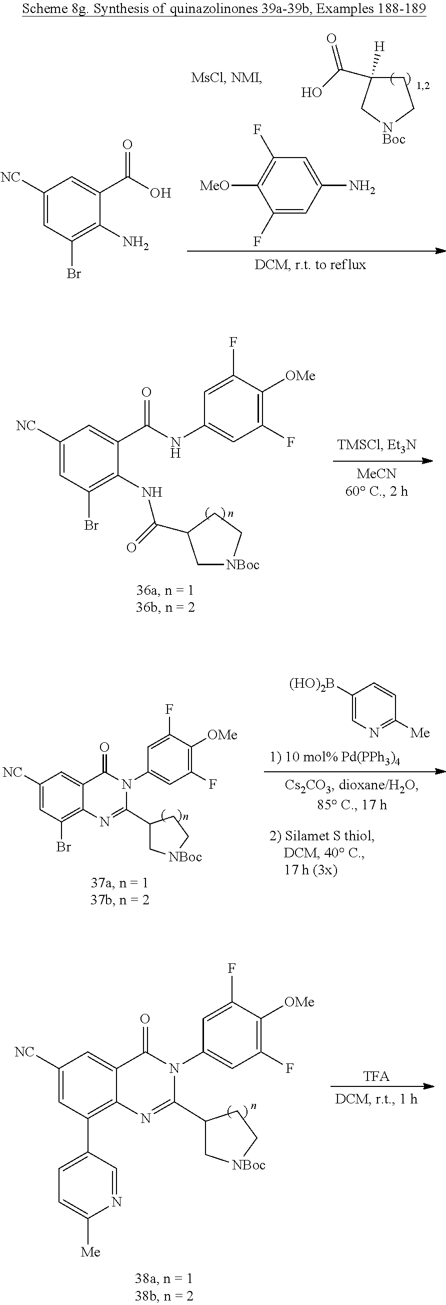

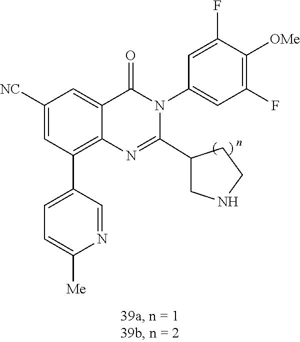

### Example 188. Synthesis of 3-(3,5-difluoro-4-methoxyphenyl)-8-(6-methylpyridin-3-yl)-4-oxo-2-(pyrrolidin-3-yl)-3,4-dihydroquinazoline-6-carbonitrile (39a)

Prepared as shown in Scheme 8g.

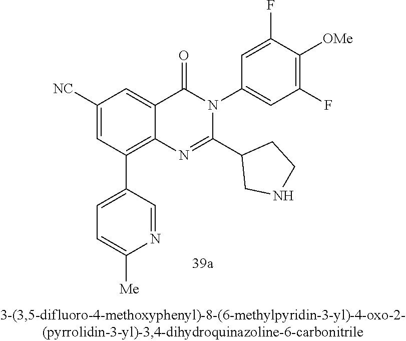 [SMILES: COc1c(F)cc(-n2c(C3CCNC3)nc3c(-c4ccc(C)nc4)cc(C#N)cc3c2=O)cc1F]

### Synthesis of tert-butyl 3-((2-bromo-4-cyano-6-((3,5-difluoro-4-methoxyphenyl)carbamoyl)phenyl)carbamoyl)pyrrolidine-1-carboxylate (36a)

(S)-1-(tert-butoxycarbonyl)pyrrolidine-3-carboxylic acid (660 mg, 3.1 mmol, 1.34 equiv.) was added to an oven-dried round-bottom flask with stir bar. The flask was fitted with a septum and placed under nitrogen. DCM (12 mL), and NMI (0.74 mL, 9.3 mmol, 3 equiv.) were added via syringe. MsCl (0.24 mL, 3.1 mmol, 1.34 equiv) was added via syringe and the reaction was stirred at room temperature for 1 h. 2-amino-3-bromo-5-cyanobenzoic acid (532 mg, 2.32 mmol, 1 equiv.) was added in one portion, a reflux condenser was fitted to the flask, and the reaction was heated to reflux for 17 h. After 17 h, 3,5-difluoro-4-methoxyaniline (493 mg, 3.1 mmol, 1.34 equiv) was added and the reaction was refluxed for an additional 17 h. after 17 h, the reaction was cooled to room temperature and quenched with sat. aq. NH₄Cl (12 mL). The reaction mixture was transferred to a separatory funnel and the layers were shaken and separated. The aqueous layer was washed with DCM (2×, 12 mL each) and the combined organics were washed with brine (1×, 50 mL). The combined organics were dried over MgSO₄, filtered, and concentrated. Purified via silica gel chromatography (4:1 to 1:1 hexanes:EtOAc). tert-butyl 3-((2-bromo-4-cyano-6-((3,5-difluoro-4-methoxyphenyl)carbamoyl)phenyl)carbamoyl) pyrrolidine-1-carboxylate (36a) obtained as a brown solid (60 mg, 0.1 mmol, 3% yield). ¹H NMR (400 MHz, DMSO-d₆) δ 10.69 (s, 1H), 10.23 (s, 1H), 8.46 (s, 1H), 8.11 (s, 1H), 7.43 (d, J=10.5 Hz, 2H), 3.89 (s, 3H), 3.28-3.15 (m, 4H), 2.13-1.86 (m, 3H), 1.36 (s, 9H) (Note: some peaks obscured by solvent).

Compound 36a (60 mg, 0.1 mmol, 1 equiv.) was added to a vial with stir bar and the vial was fitted with a septum. An N₂ balloon was added and the vial was purged. Dry MeCN (2 mL) was added via syringe followed by Et₃N (0.2 mL, 1.5 mmol, 6 equiv.) and TMSCl (160 μL, 1.2 mmol, 5 equiv.). The reaction was heated to 60° C. for 2 h. After 2 h, the reaction was cooled to room temperature and quenched with sat. aq. NH₄Cl (2 mL). The layers were shaken and separated. The aqueous layer was washed with DCM (2×, 2 mL each) and the combined organics were washed with brine (1×, 5 mL). The combined organics were dried over MgSO₄, filtered, and concentrated. Purified via silica gel chromatography (3:1 hexanes:EtOAc). tert-butyl 3-(8-bromo-6-cyano-3-(3,5-difluoro-4-methoxyphenyl)-4-oxo-3,4-dihydroquinazolin-2-yl)pyrrolidine-1-carboxylate (37a) obtained as a yellow film (44 mg, 0.078 mmol, 78% yield). ¹H NMR (400 MHz, CDCl₃) δ 8.49 (d, J=1.8 Hz, 1H), 8.27-8.19 (m, 1H), 6.87 (d, J=9.4 Hz, 2H), 4.13 (s, 3H), 3.82-3.67 (m, 2H), 3.67-3.47 (m, 1H), 3.43-3.30 (m, 1H), 3.15 (p, J=7.1 Hz, 1H), 2.54-2.24 (m, 1H), 2.09 (dt, J=12.5, 6.2 Hz, 1H), 1.45 (s, 9H).

A microwave vial with stir bar was charged with bromide 37a (44 mg, 0.078 mmol, 1 equiv.), boronic acid (12 mg, 0.086 mmol, 1.1 equiv.), Cs₂CO₃ (50 mg, 0.156 mmol, 2 equiv.), and Pd(PPh₃)₄ (9 mg, 0.0078 mmol, 0.1 equiv.). A 4:1 mixture of 1,4-dioxane:H₂O was added via syringe (1 mL). The vial was sealed with a septum and the mixture was sparged with N₂ for 15 min. After 15 min, the reaction was heated to 80° C. for 17 h. After 17 h, the reaction was cooled to room temperature and diluted with EtOAc and H₂O (2 mL each). The layers were shaken and separated. The aqueous phase was washed twice with EtOAc (2 mL each). The combined organics were dried over Na₂SO₄, filtered, and concentrated. The crude reaction mixture was purified via silica gel chromatography (3:1 to 1:2 hexanes:EtOAc). Trace palladium was removed by dissolving the purified product in DCM (0.2 M) and adding SiliametS Thiol Metal Scavenger (33 wt %) to the solution. The mixture was heated to 40° C. for 8 h before being filtered through celite. This procedure was repeated two more times. tert-butyl 3-(6-cyano-3-(3,5-difluoro-4-methoxyphenyl)-8-(6-methylpyridin-3-yl)-4-oxo-3,4-dihydroquinazolin-2-yl)pyrrolidine-1-carboxylate (38a) obtained as a white film (25 mg, 0.044 mmol, 56% yield). ¹H NMR (400 MHz, Chloroform-d) δ 8.72 (s, 1H), 8.59 (s, 1H), 7.99 (s, 1H), 7.83 (d, J=8.1 Hz, 1H), 7.32 (d, J=8.0 Hz, 1H), 6.88 (d, J=9.1 Hz, 2H), 4.13 (s, 3H), 3.56-3.37 (m, 3H), 3.32-3.05 (m, 2H), 2.66 (s, 3H), 2.35-2.18 (m, 1H), 2.04-1.94 (m, 1H), 1.42 (s, 9H).

### Synthesis of 3-(3,5-difluoro-4-methoxyphenyl)-8-(6-methylpyridin-3-yl)-4-oxo-2-(pyrrolidin-3-yl)-3,4-dihydroquinazoline-6-carbonitrile (39a)

Compound 38a (25 mg, 0.044 mmol, 1 equiv.) was added to a vial with stir bar and dissolved in DCM (0.4 mL). TFA (0.16 mL) was added via syringe and the reaction was stirred at room temperature for 1 h. After 1 h, the reaction was diluted with DCM (3 mL) and quenched with sat. aq. NaHCO₃ (3 mL). The layers were shaken and separated. The aqueous phase was washed twice with DCM (2 mL each). The combined organics were dried over Na₂SO₄, filtered, and concentrated. The crude reaction mixture was purified via silica gel chromatography (0 to 5% 4:1 MeOH:sat. aq. NH₄OH in DCM). 3-(3,5-difluoro-4-methoxyphenyl)-8-(6-methylpyridin-3-yl)-4-oxo-2-(pyrrolidin-3-yl)-3,4-dihydroquinazoline-6-carbonitrile (39a) obtained as a yellow film (15 mg, 0.031 mmol, 70% yield). ¹H NMR (400 MHz, CDCl₃) δ 8.72 (s, 1H), 8.58 (s, 1H), 7.98 (s, 1H), 7.82 (d, J=7.8 Hz, 1H), 7.30 (d, J=8.0 Hz, 1H), 6.92-6.81 (m, 2H), 4.12 (s, 3H), 3.16 (d, J=11.0 Hz, 1H), 3.09-2.88 (m, 3H), 2.81 (q, J=7.5, 6.6 Hz, 1H), 2.66 (s, 3H), 2.07-1.97 (m, 2H). MS (ESI) m/z: 474.2 (M+1)⁺.

### Example 189. Synthesis of 3-(3,5-difluoro-4-methoxyphenyl)-8-(6-methylpyridin-3-yl)-4-oxo-2-(piperidin-3-yl)-3,4-dihydroquinazoline-6-carbonitrile (39b)

Prepared as shown in Scheme 8g.

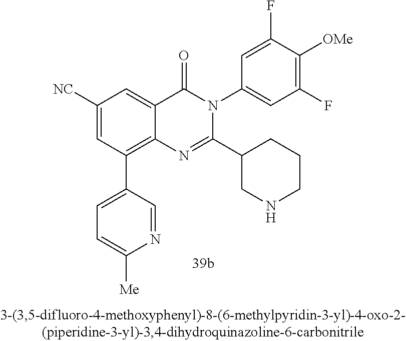 [SMILES: COc1c(F)cc(-n2c(C3CCCNC3)nc3c(-c4ccc(C)nc4)cc(C#N)cc3c2=O)cc1F]

1-(tert-butoxycarbonyl)piperidine-3-carboxylic acid (713 mg, 3.11 mmol, 1.5 equiv.) was added to an oven-dried round-bottom flask with stir bar. The flask was fitted with a septum and placed under nitrogen. DCE (10 mL), and NMI (0.496 mL, 6.22 mmol, 3 equiv.) were added via syringe. MsCl (0.24 mL, 3.1 mmol, 1.5 equiv) was added via syringe and the reaction was stirred at room temperature for 1 h. 2-amino-3-bromo-5-cyanobenzoic acid (500 mg, 2.07 mmol, 1 equiv.) was added in one portion, a reflux condenser was fitted to the flask, and the reaction was heated to reflux for 17 h. After 17 h, 3,5-difluoro-4-methoxyaniline (495 mg, 3.1 mmol, 1.5 equiv) was added and the reaction was refluxed for an additional 17 h. after 17 h, the reaction was cooled to room temperature and quenched with sat. aq. NH₄Cl (10 mL). The reaction mixture was transferred to a separatory funnel and the layers were shaken and separated. The aqueous layer was washed with DCM (2×, 10 mL each) and the combined organics were washed with brine (1×, 50 mL). The combined organics were dried over MgSO₄, filtered, and concentrated. Purified via silica gel chromatography (3:1 to 1:1 hexanes:EtOAc). tert-butyl 3-((2-bromo-4-cyano-6-((3,5-difluoro-4-methoxyphenyl)carbamoyl)phenyl)carbamoyl)piperidine-1-carboxylate (36b) obtained as a brown-white foam. 36b was pushed forward without further purification.

Amide 36b (150 mg, 0.25 mmol, 1 equiv.) was added to a vial with stir bar and the vial was fitted with a septum. An N₂ balloon was added and the vial was purged. Dry MeCN (2 mL) was added via syringe followed by Et₃N (0.2 mL, 1.5 mmol, 6 equiv.) and TMSCl (160 μL, 1.25 mmol, 5 equiv.). The reaction was heated to 60° C. for 2 h. After 2 h, the reaction was cooled to room temperature and quenched with sat. aq. NH₄Cl (2 mL). The layers were shaken and separated. The aqueous layer was washed with DCM (2×, 2 mL each) and the combined organics were washed with brine (1×, 5 mL). The combined organics were dried over MgSO₄, filtered, and concentrated. Purified via silica gel chromatography (3:1 hexanes:EtOAc). tert-butyl 3-(8-bromo-6-cyano-3-(3,5-difluoro-4-methoxyphenyl)-4-oxo-3,4-dihydroquinazolin-2-yl)piperidine-1-carboxylate (37b) obtained as a yellow-white foam (83 mg, 0.14 mmol, 7% yield over 2 steps). 1H NMR (400 MHz, CDCl₃) δ 8.49 (dd, J=2.6, 1.5 Hz, 1H), 8.22 (d, J=1.7 Hz, 1H), 7.08-6.72 (m, 2H), 4.11 (m, 4H), 3.31-3.16 (m, 1H), 3.00-2.70 (m, 1H), 2.60-2.40 (m, 1H), 2.02-1.72 (m, 3H), 1.40 (s, 9H), 1.26 (t, J=7.1 Hz, 2H).

### Tert-butyl 3-(6-cyano-3-(3,5-difluoro-4-methoxyphenyl)-8-(6-methylpyridin-3-yl)-4-oxo-3,4-dihydroquinazolin-2-yl)piperidine-1-carboxylate (38b)

A microwave vial with stir bar was charged with bromide 37b (83 mg, 0.14 mmol, 1 equiv.), boronic acid (22 mg, 0.15 mmol, 1.1 equiv.), Cs₂CO₃ (91 mg, 0.28 mmol, 2 equiv.), and Pd(PPh₃)₄ (16 mg, 0.014 mmol, 0.1 equiv.). A 4:1 mixture of 1,4-dioxane:H₂O was added via syringe (2 mL). The vial was sealed with a septum and the mixture was sparged with N₂ for 15 min. After 15 min, the reaction was heated to 80° C. for 17 h. After 17 h, the reaction was cooled to room temperature and diluted with EtOAc and H₂O (4 mL each). The layers were shaken and separated. The aqueous phase was washed twice with EtOAc (4 mL each). The combined organics were dried over Na₂SO₄, filtered, and concentrated. The crude reaction mixture was purified via silica gel chromatography (3:1 to 1:2 hexanes:EtOAc). Trace palladium was removed by dissolving the purified product in DCM (0.2 M) and adding SiliametS Thiol Metal Scavenger (33 wt %) to the solution. The mixture was heated to 40° C. for 8 h before being filtered through celite. This procedure was repeated two more times. tert-butyl 3-(6-cyano-3-(3,5-difluoro-4-methoxyphenyl)-8-(6-methylpyridin-3-yl)-4-oxo-3,4-dihydroquinazolin-2-yl)piperidine-1-carboxylate (38b) obtained as a white film (55 mg, 0.094 mmol, 67% yield). ¹H NMR (400 MHz, Chloroform-d) δ 8.78 (d, J=2.0 Hz, 1H), 8.59 (d, J=1.9 Hz, 1H), 7.98 (d, J=1.9 Hz, 1H), 7.84 (dd, J=8.0, 2.3 Hz, 1H), 7.32 (d, J=8.0 Hz, 1H), 7.01-6.81 (m, 2H), 4.17-4.06 (m, 4H), 2.91 (t, J=11.7 Hz, 1H), 2.67 (s, 3H), 2.45 (t, J=10.7 Hz, 1H), 1.94-1.53 (m, 4H), 1.39 (s, 9H), 1.26 (t, J=7.1 Hz, 2H).

3-(3,5-difluoro-4-methoxyphenyl)-8-(6-methylpyridin-3-yl)-4-oxo-2-(pyrrolidin-3-yl)-3,4-dihydroquinazoline-6-carbonitrile (39b): To a vial containing 38b (55 mg, 0.094 mmol, 1 equiv.) with stir bar was added DCM (1 mL). TFA (0.4 mL) was added via syringe and the reaction was stirred at room temperature for 1 h. After 1 h, the reaction was diluted with DCM (3 mL) and quenched with sat. aq. NaHCO₃ (3 mL). The layers were shaken and separated. The aqueous phase was washed twice with DCM (2 mL each). The combined organics were dried over Na₂SO₄, filtered, and concentrated. The crude reaction mixture was purified via silica gel chromatography (0 to 5% (4:1 MeOH:sat. aq. NH₄OH) in DCM). 3-(3,5-difluoro-4-methoxyphenyl)-8-(6-methylpyridin-3-yl)-4-oxo-2-(pyrrolidin-3-yl)-3,4-dihydroquinazoline-6-carbonitrile (39b) obtained as a clear film (36 mg, 0.074 mmol, 79% yield). 1H NMR (400 MHz, CDCl₃) δ 8.79 (s, 1H), 8.57 (s, 1H), 7.98 (s, 1H), 7.86 (d, J=7.9 Hz, 1H), 7.32 (d, J=8.0 Hz, 1H), 6.87 (s, 2H), 4.12 (s, 3H), 3.07 (d, J=12.0 Hz, 1H), 2.89 (dt, J=22.2, 11.5 Hz, 2H), 2.67 (s, 3H), 2.55 (q, J=11.3, 10.2 Hz, 2H), 1.88 (d, J=12.8 Hz, 1H), 1.69 (dt, J=20.7, 12.7 Hz, 3H), 1.28 (d, J=14.0 Hz, 1H). MS (ESI) m/z: 488.2 (M+1)⁺.

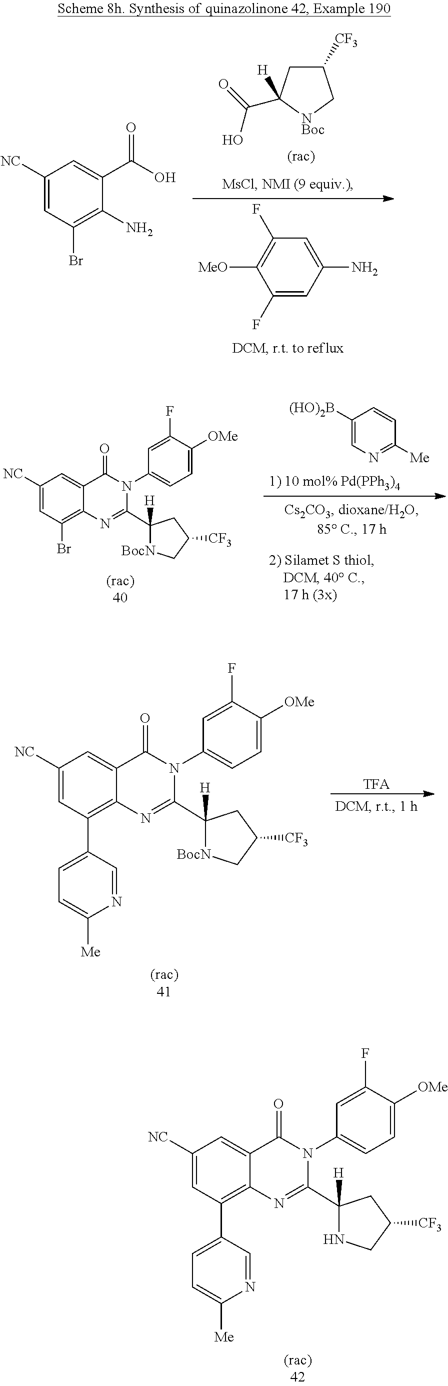

### Example 190. Synthesis of 3-(3,5-difluoro-4-methoxyphenyl)-8-(6-methylpyridin-3-yl)-4-oxo-2-(piperidin-3-yl)-3,4-dihydroquinazoline-6-carbonitrile (42)

Prepared as shown in Scheme 8h.

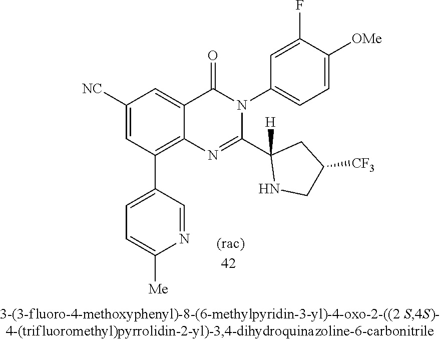 [SMILES: COc1ccc(-n2c([C@@H]3C[C@H](C(F)(F)F)CN3)nc3c(-c4ccc(C)nc4)cc(C#N)cc3c2=O)cc1F]

### Tert-butyl cis-(±)-2-(8-bromo-6-cyano-3-(3-fluoro-4-methoxyphenyl)-4-oxo-3,4-dihydroquinazolin-2-yl)-4-(trifluoromethyl)pyrrolidine-1-carboxylate (40)

cis-(±)-1-(tert-butoxycarbonyl)-4-(trifluoromethyl)pyrrolidine-2-carboxylic acid (204 mg, 0.72 mmol, 1.5 equiv.) was added to an oven-dried round-bottom flask with stir bar. The flask was fitted with a septum and placed under nitrogen. DCM (5.14 mL), and NMI (0.172 mL, 2.16 mmol, 3 equiv.) were added via syringe. MsCl (0.056 mL, 0.72 mmol, 1.5 equiv) was added via syringe and the reaction was stirred at room temperature for 1 h. 2-amino-3-bromo-5-cyanobenzoic acid (116 mg, 0.48 mmol, 1 equiv.) was added in one portion, followed by NMI (0.344 mL, 4.32 mmol, 9 equiv.) via syringe, a reflux condenser was fitted to the flask, and the reaction was heated to reflux for 17 h. After 17 h, 3-fluoro-4-methoxyaniline (102 mg, 0.72 mmol, 1.5 equiv) was added and the reaction was refluxed for an additional 17 h. after 17 h, the reaction was cooled to room temperature and quenched with sat. aq. NH₄Cl (10 mL). The reaction mixture was transferred to a separatory funnel and the layers were shaken and separated. The aqueous layer was washed with DCM (2×, 10 mL each) and the combined organics were washed with brine (1×, 50 mL). The combined organics were dried over MgSO₄, filtered, and concentrated. Purified via silica gel chromatography (0:1 to 1:19 EtOAc:DCM). tert-butyl cis-(±)-2-(8-bromo-6-cyano-3-(3-fluoro-4-methoxyphenyl)-4-oxo-3,4-dihydroquinazolin-2-yl)-4-(trifluoromethyl)pyrrolidine-1-carboxylate (40) obtained as a white solid (47 mg, 0.077 mmol, 16% yield). (Mixture of rotamers)¹H NMR (400 MHz, CDCl₃) δ 8.63-8.40 (m, 1H), 8.31-8.11 (m, 1H), 7.42-7.30 (m, 1H), 7.24-7.03 (m, 1H), 7.03-6.87 (m, 1H), 4.80-4.55 (m, 1H), 4.30-4.08 (m, 1H), 3.99 (dd, J=9.7, 1.6 Hz, 3H), 3.94-3.43 (m, 2H), 2.43-1.97 (m, 2H), 1.37 (dd, J=44.4, 3.4 Hz, 9H).

### Tert-butyl cis-(±)-2-(6-cyano-3-(3-fluoro-4-methoxyphenyl)-8-(6-methylpyridin-3-yl)-4-oxo-3,4-dihydroquinazolin-2-yl)-4-(trifluoromethyl)pyrrolidine-1-carboxylate (41)

A microwave vial with stir bar was charged with bromide 40 (37 mg, 0.061 mmol, 1 equiv.), boronic acid (9 mg, 0.067 mmol, 1.1 equiv.), Cs₂CO₃ (40 mg, 0.122 mmol, 2 equiv.), and Pd(PPh₃)₄ (7 mg, 0.0061 mmol, 0.1 equiv.). A 4:1 mixture of 1,4-dioxane:H₂O was added via syringe (0.87 mL). The vial was sealed with a septum and the mixture was sparged with N₂ for 15 min. After 15 min, the reaction was heated to 80° C. for 17 h. After 17 h, the reaction was cooled to room temperature and diluted with EtOAc and H₂O (4 mL each). The layers were shaken and separated. The aqueous phase was washed twice with EtOAc (4 mL each). The combined organics were dried over Na₂SO₄, filtered, and concentrated. The crude reaction mixture was purified via silica gel chromatography (3:1 to 1:1 hexanes:EtOAc). Trace palladium was removed by dissolving the purified product in DCM (0.2 M) and adding SiliametS Thiol Metal Scavenger (33 wt %) to the solution. The mixture was heated to 40° C. for 8 h before being filtered through celite. This procedure was repeated two more times. tert-butyl cis-(±)-2-(6-cyano-3-(3-fluoro-4-methoxyphenyl)-8-(6-methylpyridin-3-yl)-4-oxo-3,4-dihydroquinazolin-2-yl)-4-(trifluoromethyl)pyrrolidine-1-carboxylate (41) obtained as a white solid (11 mg, 0.018 mmol, 30% yield) (Mixture of rotamers)¹H NMR (400 MHz, CDCl₃) δ 8.60-8.51 (m, 2H), 8.00-7.88 (m, 1H), 7.85-7.70 (m, 1H), 7.33-7.23 (m, 1H), 7.16-6.82 (m, 3H), 4.72-4.38 (m, 1H), 3.98-3.85 (m, 3H), 3.61-3.24 (m, 2H), 3.10-2.71 (m, 1H), 2.59 (d, J=3.7 Hz, 3H), 2.18-1.80 (m, 2H), 1.29 (d, J=10.3 Hz, 9H).

### cis-(±)-3-(3-fluoro-4-methoxyphenyl)-8-(6-methylpyridin-3-yl)-4-oxo-2-((2S,4S)-4-(trifluoromethyl) pyrrolidin-2-yl)-3,4-dihydroquinazoline-6-carbonitrile (42)

Compound 41 (11 mg, 0.018 mmol, 1 equiv.) was added to a vial with stir bar and dissolved in DCM (0.4 mL). TFA (0.16 mL) was added via syringe and the reaction was stirred at room temperature for 1 h. After 1 h, the reaction was diluted with DCM (3 mL) and quenched with sat. aq. NaHCO₃ (3 mL). The layers were shaken and separated. The aqueous phase was washed twice with DCM (2 mL each). The combined organics were dried over Na₂SO₄, filtered, and concentrated. The crude reaction mixture was purified via silica gel chromatography (0 to 5% (4:1 MeOH:sat. aq. NH₄OH) in DCM). cis-(±)-3-(3-fluoro-4-methoxyphenyl)-8-(6-methylpyridin-3-yl)-4-oxo-2-((2S,4S)-4-(trifluoromethyl)pyrrolidin-2-yl)-3,4-dihydroquinazoline-6-carbonitrile (42) obtained as a clear film (6 mg, 0.011 mmol, 61% yield). ¹H NMR (400 MHz, CDCl₃)) δ 8.72 (d, J=2.1 Hz, 1H), 8.61 (d, J=1.9 Hz, 1H), 7.98 (d, J=1.9 Hz, 1H), 7.80 (dd, J=8.0, 2.3 Hz, 1H), 7.31 (d, J=8.0 Hz, 1H), 7.18-7.10 (m, 2H), 7.05-6.96 (m, 1H), 4.06 (ddd, J=12.2, 7.9, 5.2 Hz, 1H), 3.98 (s, 3H), 3.21 (ddd, J=11.8, 8.0, 1.8 Hz, 1H), 2.95-2.86 (m, 1H), 2.82 (tt, J=15.5, 7.5 Hz, 1H), 2.67 (s, 3H), 2.24-2.12 (m, 1H), 1.96-1.86 (m, 1H). MS (ESI) m/z: 524.2 (M+1)⁺.

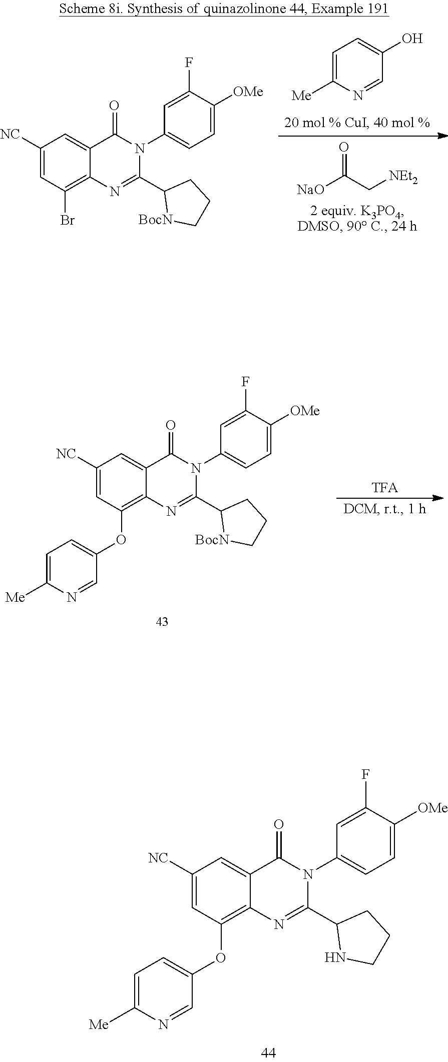

### Example 191. Synthesis of 3-(3-fluoro-4-methoxyphenyl)-8-((6-methylpyridin-3-yl)oxy)-4-oxo-2-(pyrrolidin-2-yl)-3,4-dihydroquinazoline-6-carbonitrile (44)

Prepared as shown in Scheme 8i.

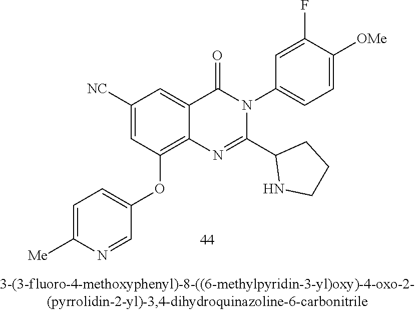 [SMILES: COc1ccc(-n2c(C3CCCN3)nc3c(Oc4ccc(C)nc4)cc(C#N)cc3c2=O)cc1F]

### Tert-butyl 2-(6-cyano-3-(3-fluoro-4-methoxyphenyl)-8-((6-methylpyridin-3-yl)oxy)-4-oxo-3,4-dihydroquinazolin-2-yl)pyrrolidine-1-carboxylate (43)

A microwave vial with stir bar was charged with bromide 22d (150 mg, 0.28 mmol, 1 equiv.), 5-hydroxy-2-methylpyridine (45 mg, 0.41 mmol, 1.5 equiv.), K₃PO₄ (119 mg, 0.056 mmol, 2 equiv.), N,N-diethylglycine sodium salt (17 mg, 0.112 mmol, 0.4 equiv.), and CuI (11 mg, 0.0056 mmol, 0.2 equiv.). DMSO was added via syringe (0.64 mL). The vial was sealed with a septum and the mixture was sparged with N₂ for 15 min. After 15 min, the reaction was heated to 90° C. for 24 h. After 24 h, the reaction was cooled to room temperature and diluted with sat. aq. NaHCO₃ (2 mL), EtOAc (2 mL). The layers were shaken and separated. The aqueous phase was washed twice with EtOAc (2 mL each). The combined organics were dried over MgSO₄, filtered, and concentrated. The crude reaction mixture was purified twice via silica gel chromatography (1^{st} column, 4:1 to 1:1 hexanes:EtOAc, 2^{nd} column, 0:1 to 1:19 MeOH:DCM). tert-butyl 2-(6-cyano-3-(3-fluoro-4-methoxyphenyl)-8-((6-methylpyridin-3-yl)oxy)-4-oxo-3,4-dihydroquinazolin-2-yl)pyrrolidine-1-carboxylate (43) isolated as an impure mixture that was pushed forward to the next step without further purification (˜17 mg). 3-(3-fluoro-4-methoxyphenyl)-8-((6-methylpyridin-3-yl)oxy)-4-oxo-2-(pyrrolidin-2-yl)-3,4-dihydroquinazoline-6-carbonitrile (44): Compound 43 (17 mg) was added to a vial with stir bar and dissolved in DCM (0.4 mL). TFA (0.16 mL) was added via syringe and the reaction was stirred at room temperature for 1 h. After 1 h, the reaction was diluted with DCM (3 mL) and quenched with sat. aq. NaHCO₃ (3 mL). The layers were shaken and separated. The aqueous phase was washed twice with DCM (2 mL each). The combined organics were dried over Na₂SO₄, filtered, and concentrated. The crude reaction mixture was purified via silica gel chromatography (0 to 5% (4:1 MeOH:sat. aq. NH₄OH) in DCM). 3-(3-fluoro-4-methoxyphenyl)-8-((6-methylpyridin-3-yl)oxy)-4-oxo-2-(pyrrolidin-2-yl)-3,4-dihydroquinazoline-6-carbonitrile (44) isolated as an off-white film (5 mg, 0.011 mmol, 4% yield over 2 steps)¹H NMR (400 MHz, CDCl₃) δ 8.36 (d, J=1.8 Hz, 1H), 8.32 (t, J=2.2 Hz, 1H), 7.59-7.54 (m, 1H), 7.25-7.15 (m, 2H), 7.14-7.03 (m, 2H), 7.03-6.94 (m, 1H), 3.97 (s, 3H), 3.87-3.72 (m, 1H), 3.05-2.86 (m, 1H), 2.65 (dq, J=12.9, 6.4 Hz, 1H), 2.57 (s, 3H), 1.79-1.48 (m, 4H). MS (ESI) m/z: 471.5 (M).

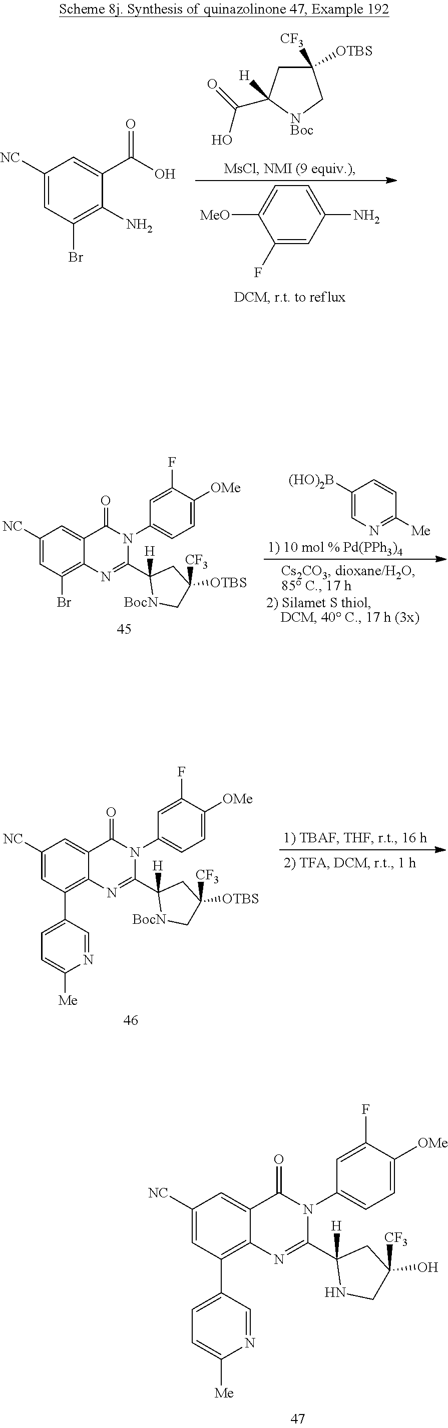

### Example 192. Synthesis of 3-(3-fluoro-4-methoxyphenyl)-2-((2S,4S)-4-hydroxy-4-(trifluoromethyl)pyrrolidin-2-yl)-8-(6-methylpyridin-3-yl)-4-oxo-3,4-dihydroquinazoline-6-carbonitrile (47)

Prepared as shown in Scheme 8j.

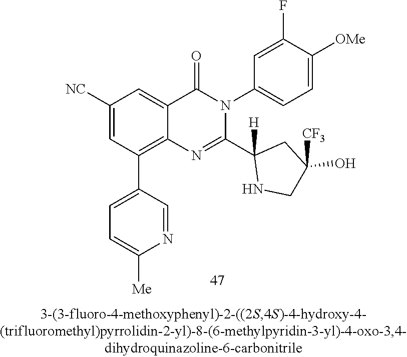 [SMILES: COc1ccc(-n2c([C@@H]3C[C@@](O)(C(F)(F)F)CN3)nc3c(-c4ccc(C)nc4)cc(C#N)cc3c2=O)cc1F]

### Synthesis of tert-butyl (2S,4S)-2-(8-bromo-6-cyano-3-(3-fluoro-4-methoxyphenyl)-4-oxo-3,4-dihydroquinazolin-2-yl)-4-((tert-butyldimethylsilyl)oxy)-4-(trifluoromethyl)pyrrolidine-1-carboxylate (45)

(2S,4S)-1-(tert-butoxycarbonyl)-4-((tert-butyldimethylsilyl)oxy)-4-(trifluoromethyl)pyrrolidine-2-carboxylic acid (298 mg, 0.72 mmol, 1.5 equiv.) was added to an oven-dried round-bottom flask with stir bar. The flask was fitted with a septum and placed under nitrogen. DCM (5.14 mL), and NMI (0.172 mL, 2.16 mmol, 3 equiv.) were added via syringe. MsCl (0.056 mL, 0.72 mmol, 1.5 equiv) was added via syringe and the reaction was stirred at room temperature for 1 h. 2-amino-3-bromo-5-cyanobenzoic acid (116 mg, 0.48 mmol, 1 equiv.) was added in one portion, followed by NMI (0.344 mL, 4.32 mmol, 9 equiv.) via syringe, a reflux condenser was fitted to the flask, and the reaction was heated to reflux for 17 h. After 17 h, 3-fluoro-4-methoxyaniline (102 mg, 0.72 mmol, 1.5 equiv) was added and the reaction was refluxed for an additional 17 h. after 17 h, the reaction was cooled to room temperature and quenched with sat. aq. NH₄Cl (10 mL). The reaction mixture was transferred to a separatory funnel and the layers were shaken and separated. The aqueous layer was washed with DCM (2×, 10 mL each) and the combined organics were washed with brine (1×, 50 mL). The combined organics were dried over MgSO₄, filtered, and concentrated. Purified twice via silica gel chromatography (1st column, 4:1 to 2:1 hexanes:EtOAc, 2^{nd} column, 0:1 to 1:19 EtOAc:DCM). tert-butyl (2S,4S)-2-(8-bromo-6-cyano-3-(3-fluoro-4-methoxyphenyl)-4-oxo-3,4-dihydroquinazolin-2-yl)-4-((tert-butyldimethylsilyl)oxy)-4-(trifluoromethyl)pyrrolidine-1-carboxylate (45) obtained as a white solid (179 mg, 0.24 mmol, 50% yield). (Mixture of rotamers)¹H NMR (400 MHz, CDCl₃) δ 8.51 (dt, J=8.4, 1.6 Hz, 1H), 8.25-8.16 (m, 1H), 7.45-7.30 (m, 1H), 7.23-7.07 (m, 1H), 6.97-6.85 (m, 1H), 4.72-4.42 (m, 1H), 4.12-3.67 (m, 5H), 2.64-2.26 (m, 1H), 2.22-1.99 (m, 1H), 1.34 (m, 9H), 0.86-0.66 (m, 9H), 0.06 (m, 6H).

### Synthesis of tert-butyl (2S,4S)-4-((tert-butyldimethylsilyl)oxy)-2-(6-cyano-3-(3-fluoro-4-methoxyphenyl)-8-(6-methylpyridin-3-yl)-4-oxo-3,4-dihydroquinazolin-2-yl)-4-(trifluoromethyl)pyrrolidine-1-carboxylate (46)

A microwave vial with stir bar was charged with bromide 45 (159 mg, 0.21 mmol, 1 equiv.), boronic acid (32 mg, 0.23 mmol, 1.1 equiv.), Cs₂CO₃ (137 mg, 0.42 mmol, 2 equiv.), and Pd(PPh₃)₄ (24 mg, 0.021 mmol, 0.1 equiv.). A 4:1 mixture of 1,4-dioxane:H₂O was added via syringe (3 mL). The vial was sealed with a septum and the mixture was sparged with N₂ for 15 min. After 15 min, the reaction was heated to 80° C. for 17 h. After 17 h, the reaction was cooled to room temperature and diluted with EtOAc and H₂O (4 mL each). The layers were shaken and separated. The aqueous phase was washed twice with EtOAc (4 mL each). The combined organics were dried over Na₂SO₄, filtered, and concentrated. The crude reaction mixture was purified via silica gel chromatography (4:1 to 1:1 hexanes:EtOAc). Trace palladium was removed by dissolving the purified product in DCM (0.2 M) and adding SiliametS Thiol Metal Scavenger (33 wt %) to the solution. The mixture was heated to 40° C. for 8 h before being filtered through celite. This procedure was repeated two more times. tert-butyl (2S,4S)-4-((tert-butyldimethylsilyl)oxy)-2-(6-cyano-3-(3-fluoro-4-methoxyphenyl)-8-(6-methylpyridin-3-yl)-4-oxo-3,4-dihydroquinazolin-2-yl)-4-(trifluoromethyl)pyrrolidine-1-carboxylate (46) obtained as a white solid (71 mg, 0.094 mmol, 45% yield) (Mixture of rotamers) ¹H NMR (400 MHz, CDCl₃) δ 8.73-8.61 (m, 2H), 8.06 (dd, J=24.5, 2.0 Hz, 1H), 8.00-7.88 (m, 1H), 7.43 (m, 1H), 7.30-7.11 (m, 2H), 7.03-6.93 (m, 1H), 4.64 (m, 1H), 4.08-3.95 (m, 3H), 3.84-3.44 (m, 2H), 2.69 (d, J=4.6 Hz, 3H), 2.34-2.02 (m, 2H), 1.43-1.23 (m, 9H), 0.81-0.66 (m, 9H), 0.09-−0.03 (m, 6H).

### Synthesis of 3-(3-fluoro-4-methoxyphenyl)-2-((2S,4S)-4-hydroxy-4-(trifluoromethyl)pyrrolidin-2-yl)-8-(6-methylpyridin-3-yl)-4-oxo-3,4-dihydroquinazoline-6-carbonitrile (47)

Compound 46 (55 mg, 0.073 mmol, 1 equiv.) was added to a vial with stir bar and dissolved in THF (0.14 mL). TBAF (1 M in THF, 0.08 mmol, 0.080 mL, 1.1 equiv.) was added via syringe and the reaction was stirred at room temperature for 16 h. After 16 h, the reaction was concentrated in vacuo and diluted with DCM (3 mL). The reaction was washed with 1N HCL, sat. aq. NaHCO₃, and brine (3 mL each). The combined organics were dried over MgSO₄, filtered, and concentrated. The crude alcohol was then used immediately in the next reaction.

The crude alcohol (˜47 mg) was added to a vial with stir bar and dissolved in DCM (0.7 mL). TFA (0.28 mL) was added via syringe and the reaction was stirred at room temperature for 1 h. After 1 h, the reaction was diluted with DCM (3 mL) and quenched with sat. aq. NaHCO₃ (3 mL). The layers were shaken and separated. The aqueous phase was washed twice with DCM (2 mL each). The combined organics were dried over Na₂SO₄, filtered, and concentrated. The crude reaction mixture was purified via silica gel chromatography (0 to 5% (4:1 MeOH:sat. aq. NH₄OH) in DCM). 3-(3-fluoro-4-methoxyphenyl)-2-((2S,4S)-4-hydroxy-4-(trifluoromethyl)pyrrolidin-2-yl)-8-(6-methylpyridin-3-yl)-4-oxo-3,4-dihydroquinazoline-6-carbonitrile (47) was obtained as a white solid (22 mg, 0.041 mmol, 56% yield over 2 steps). ¹H NMR (400 MHz, DMSO-d₆) δ 8.70 (d, J=2.1 Hz, 1H), 8.52 (t, J=1.9 Hz, 1H), 8.33 (t, J=1.7 Hz, 1H), 8.00 (dd, J=8.0, 2.3 Hz, 1H), 7.45 (td, J=11.5, 2.3 Hz, 1H), 7.40-7.25 (m, 3H), 6.11 (d, J=5.3 Hz, 1H), 4.08-3.98 (m, 1H), 3.92 (s, 3H), 3.12 (dd, J=12.2, 5.3 Hz, 1H), 2.76-2.62 (m, 1H), 2.53 (s, 3H), 2.45-2.25 (m, 1H), 1.80 (ddd, J=13.4, 7.2, 3.1 Hz, 1H). MS (ESI) m/z: 540.1 (M+1)⁺.

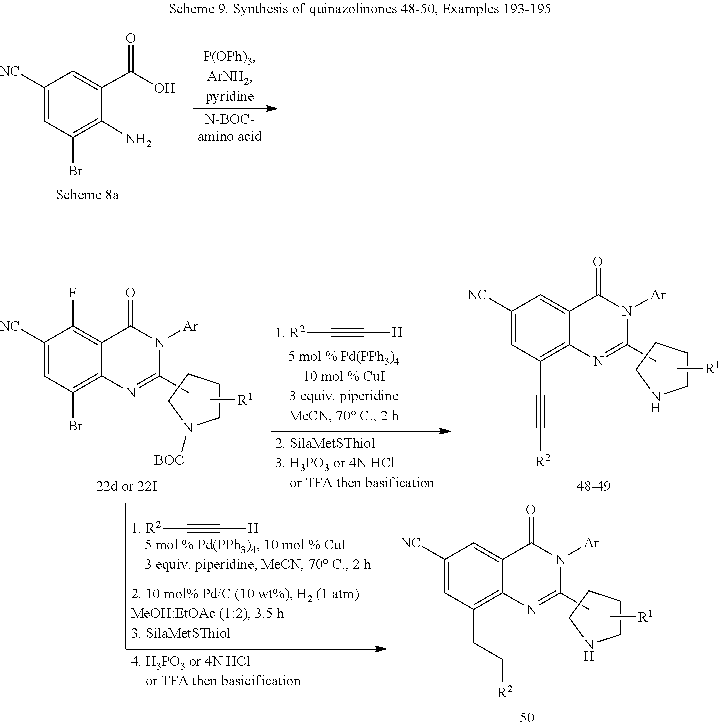

### Example 193. Synthesis of 3-(3,5-Difluoro-4-methoxyphenyl)-8-(3-hydroxy-3-methylbut-1-yn-1-yl)-4-oxo-2-(pyrrolidin-2-yl)-3,4-dihydroquinazoline-6-carbonitrile (48)

Prepared as shown in Scheme 9.

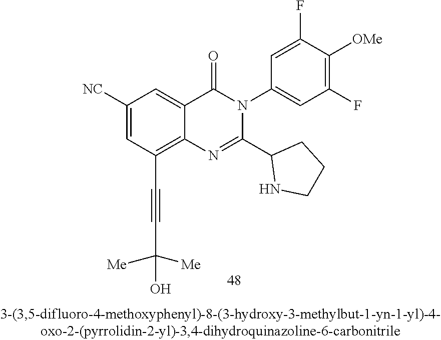 [SMILES: COc1c(F)cc(-n2c(C3CCCN3)nc3c(C#CC(C)(C)O)cc(C#N)cc3c2=O)cc1F]

Starting with bromide 22l, a Sonogashira reaction was performed according to Scheme 9 to afford the corresponding alkyl intermediate. Demetallation in triplicate (SilaMetSThiol) was performed as described for other coupled products. The BOC-protected penultimate intermediate (77.1 mg, 0.137 mmol) was deprotected in CH₂Cl₂ (1.4 mL) and trifluoroacetic acid (157 μL, 2.06 mmol) and 4N HCl in dioxane (343 μL, 1.37 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 48 (32.9 mg, 52%) as a white solid (LC/MS purity=99.4%). ¹H NMR (400 MHz, CDCl₃): δ=8.27 (d, J=1.8 Hz, 1H), 7.86 (d, J=1.8 Hz, 1H), 7.15 (d, J=10.4 Hz, 1H), 6.83 (d, J=10.3 Hz, 1H), 4.34 (brs, 1H), 4.14 (s, 3H), 4.03 (t, J=6.5 Hz, 1H), 3.32 (dt, J=11.1, 5.7 Hz, 1H), 2.90 (dt, J=11.1, 6.1 Hz, 1H), 1.94-1.74 (m, 4H), 1.63 (s, 3H), 1.63 (s, 3H).

### Example 194. Synthesis of 3-(3-Fluoro-4-methoxyphenyl)-8-((6-methylpyridin-3-yl)ethynyl)-4-oxo-2-(pyrrolidin-2-yl)-3,4-dihydroquinazoline-6-carbonitrile (49)

Prepared as shown in Scheme 9.

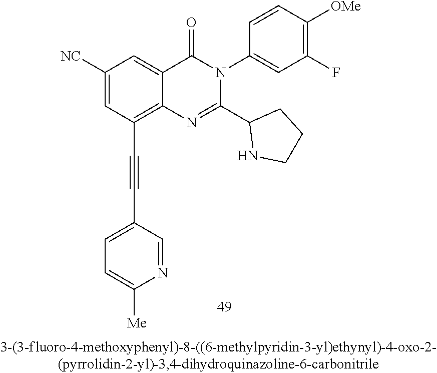 [SMILES: COc1ccc(-n2c(C3CCCN3)nc3c(C#Cc4ccc(C)nc4)cc(C#N)cc3c2=O)cc1F]

Starting with bromide 22d, a Sonogashira reaction was performed according to Scheme 9 to afford the corresponding alkyl intermediate. Demetallation in triplicate (SilaMetSThiol) was performed as described for other coupled products. The BOC-protected penultimate intermediate (53.0 mg, 0.0914 mmol) was deprotected in CH₂Cl₂ (1.0 mL) and trifluoroacetic acid (210 μL, 2.74 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 49 (39.3 mg, 90%) as a white solid (LC/MS purity=99.4%). ¹H NMR (400 MHz, CDCl₃): δ=8.71 (s, 1H), 8.47 (s, 1H), 8.12 (s, 1H), 7.75 (d, J=8.0 Hz, 1H), 7.22 (d, J=8.0 Hz, 1H), 7.16-7.11 (m, 1H), 7.10-7.00 (m, 2H), 4.02-3.95 (m, 4H), 3.48-3.25 (m, 1H), 2.94-2.84 (m, 1H), 2.77 (brs, 1H), 2.62 (s, 3H), 1.99-1.72 (m, 4H).

### Example 195. Synthesis of 3-(3-Fluoro-4-methoxyphenyl)-8-(2-(6-methylpyridin-3-yl)ethyl)-4-oxo-2-(pyrrolidin-2-yl)-3,4-dihydroquinazoline-6-carbonitrile (50)

Prepared as shown in Scheme 9.

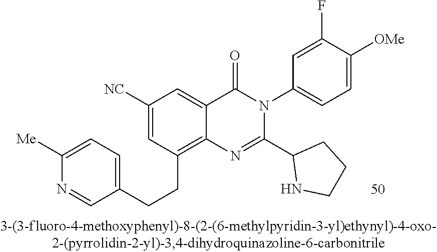 [SMILES: COc1ccc(-n2c(C3CCCN3)nc3c(CCc4ccc(C)nc4)cc(C#N)cc3c2=O)cc1F]

Prior to deprotection of compound 49, the corresponding intermediate, tert-butyl 2-(6-cyano-3-(3-fluoro-4-methoxyphenyl)-8-((6-methylpyridin-3-yl)ethynyl)-4-oxo-3,4-dihydroquinazolin-2-yl)pyrrolidine-1-carboxylate, was hydrogenated according to Scheme 9 (10% Pd/C) under hydrogen atmosphere in 1:2 MeOH/EtOAc for 3 h. After filtration through celite, the compound was demetallated (SilaMetSThiol) in triplicate as described for other analogs that underwent coupling procedures. The BOC-protected penultimate intermediate (57.4 mg, 0.0983 mmol) was deprotected in CH₂Cl₂ (1.0 mL) and trifluoroacetic acid (226 μL, 2.95 mmol). The reaction was monitored on thin-layer chromatography. After completion of the reaction, the crude mixture was concentrated in vacuo. Aqueous NaHCO₃ (sat.) was added, and the aqueous layer was extracted with CH₂Cl₂ (3 times). The organic layer was washed with aqueous solution of Na₂CO₃ (sat.) and brine, and dried over Na₂SO₄. After concentrating, the crude mixture was purified by CombiFlash gave 50 as a white solid (LC/MS purity=95.2%). ¹H NMR (400 MHz, CDCl₃): δ=8.43 (s, 1H), 8.33 (s, 1H), 7.71 (s, 1H), 7.37 (d, J=7.8 Hz, 1H), 7.20-7.04 (m, 4H), 3.98 (s, 3H), 3.96-3.91 (m, 1H), 3.43-3.29 (m, 2H), 3.29-3.20 (m, 1H), 3.07-2.96 (m, 2H), 2.93 (brs, 1H), 2.89-2.79 (m, 1H), 2.54 (s, 3H), 1.92-1.72 (m, 4H).

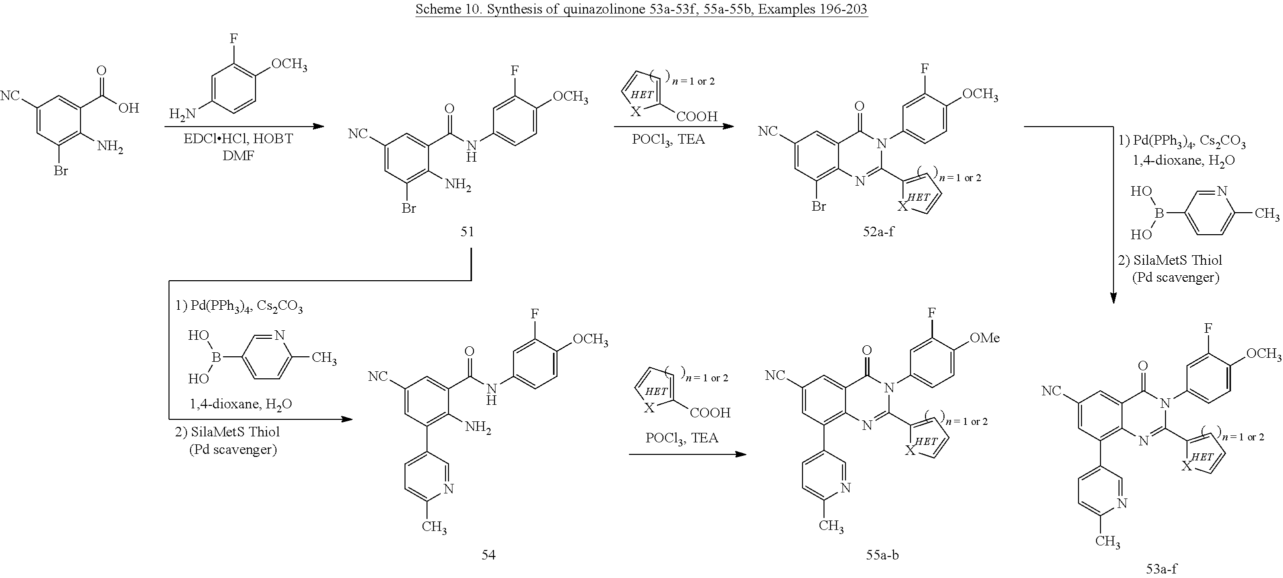

### Example 196. Synthesis of 3-(3-fluoro-4-methoxyphenyl)-8-(6-methylpyridin-3-yl)-4-oxo-2-(pyridin-2-yl)-3,4-dihydroquinazoline-6-carbonitrile (53a)

Prepared according to Scheme 10.

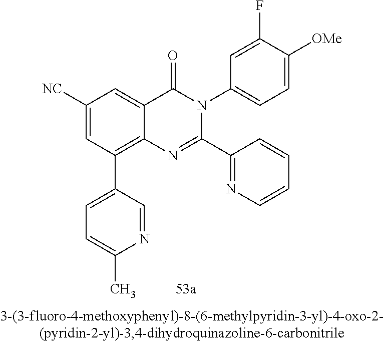 [SMILES: COc1ccc(-n2c(-c3ccccn3)nc3c(-c4ccc(C)nc4)cc(C#N)cc3c2=O)cc1F]

### Synthesis of 2-amino-3-bromo-5-cyano-N-(3-fluoro-4-methoxyphenyl)benzamide (51)

To the mixture of 2-amino-3-bromo-5-cyanobenzoic acid (10 g, 0.0417 mol) in DMF (200 mL) cooled to 0° C. was added EDCI.HCl (8.8 g, 0.0459 mol) and HOBT (7.4 g, 0.0459 mol). After being stirred at rt for 1 h, 3-fluoro-4-methoxyaniline (5.9 g, 0.0417 mol) was added into the reaction mixture, and stirred at rt for 16 h. Then, the reaction mixture was concentrated to remove DMF. The resulting residual was diluted by water (500 mL), and a lot of grey solid was generated at the same time. It was extracted with EtOAc (4×300 mL). The combined organic layers were washed with water (2×300 mL), saturated aqueous Na₂CO₃, and brine (600 mL) in turn, dried over anhydrous Na₂SO₄, and filtered, then the solvent was evaporated under reduced pressure to afford a grey solid 51 (8.4 g, 55%). ¹H NMR (400 MHz, DMSO-d₆) δ 10.33 (s, 1H), 8.09 (dd, J=18.6, 1.8 Hz, 2H), 7.64 (dd, J=13.6, 2.5 Hz, 1H), 7.44-7.34 (m, 1H), 7.27-7.11 (m, 3H), 3.82 (s, 3H).

### Synthesis of 8-bromo-3-(3-fluoro-4-methoxyphenyl)-4-oxo-2-(pyridin-2-yl)-3,4-dihydroquinazoline-6-carbonitrile (52a)

To the mixture of 51 (100 mg, 0.285 mmol) in CHCl₃ (5 mL) was added picolinic acid (41 mg, 0.33 mmol), POCl₃ (0.13 mL, 1.38 mmol), and triethylamine (0.076 mL, 0.55 mmol) in a seal tube. The reaction mixture was heated to reflux for 5 h. Then, the reaction mixture was concentrated to give the resulting residual, which was adjusted to PH=10-11 using saturated aqueous Na₂CO₃. The aqueous layer was extracted with DCM (3×20 mL). The combined organic layers were washed with brine (100 mL), dried over anhydrous Na₂SO₄, and filtered, then the solvent was evaporated under reduced pressure to afford the crude product. It was purified by flash chromatography (0% to 20% to 50% A (DCM:ethyl acetate=1:4)/Hexane) to afford 52a (47 mg, 42%) as pale yellow solid. ¹H NMR (400 MHz, Chloroform-d) δ 8.61 (d, J=1.8 Hz, 1H), 8.35 (d, J=4.8 Hz, 1H), 8.27 (d, J=1.8 Hz, 1H), 7.91-7.84 (m, 1H), 7.83-7.73 (m, 1H), 7.26-7.21 (m, 1H), 7.02-6.95 (m, 1H), 6.90-6.81 (m, 2H), 3.87 (s, 3H).

### Synthesis of 3-(3-fluoro-4-methoxyphenyl)-8-(6-methylpyridin-3-yl)-4-oxo-2-(pyridin-2-yl)-3,4-dihydroquinazoline-6-carbonitrile (53a)

A mixture of 52a (160 mg, 0.36 mmol), cesium carbonate (235 mg, 0.72 mmol), 2-methylpyridine-5-boronic acid (74 mg, 0.54 mmol) and tetrakis(tri-phenylphosphine) palladium (0) (31 mg, 0.027 mmol) in 1,4-dioxane (10 mL) and H₂O (1.5 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 60% A (DCM:ethyl acetate=1:4)/Hexane) to give the product, which was treated with a palladium scavenger (SiliaMetS Thiol, 3×30 mg), stirred at 40° C. for 8 h and at rt for 16 h, and filtered through Celite pad to remove any residual metal from the coupling reaction. Repeating this procedure of removing Pd for three times. After concentration, 53a (90 mg, 54%) was obtained as a yellow solid. ¹H NMR (400 MHz, Chloroform-d) δ 8.87 (s, 1H), 8.70 (s, 1H), 8.29 (d, J=4.7 Hz, 1H), 8.03 (s, 1H), 7.92-7.85 (m, 1H), 7.76-7.64 (m, 2H), 7.31-7.27 (m, 1H), 7.22-7.16 (m, 1H), 7.05-6.98 (m, 1H), 6.92-6.81 (m, 2H), 3.87 (s, 3H), 2.63 (s, 3H). MS (ESI) m/z: 464.0 (M+1)⁺.

### Example 197. Synthesis of 3-(3-fluoro-4-methoxyphenyl)-8-(6-methylpyridin-3-yl)-2-(oxazol-4-yl)-4-oxo-3,4-dihydroquinazoline-6-carbonitrile (53b)

Prepared according to Scheme 10.

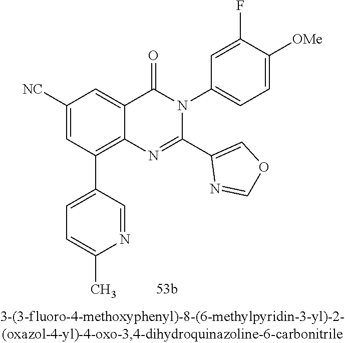 [SMILES: COc1ccc(-n2c(-c3cocn3)nc3c(-c4ccc(C)nc4)cc(C#N)cc3c2=O)cc1F]

### Synthesis of 8-bromo-3-(3-fluoro-4-methoxyphenyl)-2-(oxazol-4-yl)-4-oxo-3,4-dihydroquinazoline-6-carbonitrile (52b)

To the mixture of 51 (300 mg, 0.82 mmol) in POCl₃ (8 mL) was added oxazole-4-carboxylic acid (112 mg, 0.99 mmol), and triethylamine (0.23 mL, 1.65 mmol) in a seal tube. The reaction mixture was heated to 150° C. for 1.5 h under microwave. Then, the reaction mixture was concentrated to give the resulting residual, which was quenched by ice, and adjusted to PH=10-11 using saturated aqueous Na₂CO₃. The aqueous layer was extracted with DCM (3×20 mL). The combined organic layers were washed with brine (100 mL), dried over anhydrous Na₂SO₄, and filtered, then the solvent was evaporated under reduced pressure to afford the crude product. It was purified by flash chromatography (0% to 40% to 100% A (DCM:ethyl acetate=1:4)/Hexane) to afford 52b (160 mg, 44%) as a brown solid. ¹H NMR (400 MHz, DMSO-d₆) δ 8.70 (d, J=1.9 Hz, 1H), 8.55 (d, J=1.8 Hz, 1H), 8.37 (s, 1H), 8.03 (s, 1H), 7.42 (dd, J=11.7, 2.2 Hz, 1H), 7.29-7.18 (m, 2H), 3.90 (s, 3H).

### Synthesis of 3-(3-fluoro-4-methoxyphenyl)-8-(6-methylpyridin-3-yl)-2-(oxazol-4-yl)-4-oxo-3,4-dihydroquinazoline-6-carbonitrile (53b)

A mixture of 52b (140 mg, 0.32 mmol), cesium carbonate (209 mg, 0.64 mmol), 2-methylpyridine-5-boronic acid (57 mg, 0.42 mmol) and tetrakis(tri-phenylphosphine) palladium (0) (28 mg, 0.024 mmol) in 1,4-dioxane (5 mL) and H₂O (1 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 50% A (DCM:MeOH=100:1)/Hexane) to give the product, which was treated with a palladium scavenger (SiliaMetS Thiol, 3×20 mg), stirred at 40° C. for 8 h and at rt for 16 h, and filtered through Celite pad to remove any residual metal from the coupling reaction. Repeating this procedure of removing Pd for three times. After concentration, 53b (60 mg, 41%) was obtained as a yellow solid. ¹H NMR (400 MHz, Chloroform-d) δ 8.86 (s, 1H), 8.63 (s, 1H), 8.02 (s, 1H), 7.98-7.87 (m, 1H), 7.65 (d, J=20.5 Hz, 2H), 7.39-7.28 (m, 1H), 7.04 (s, 3H), 3.96 (s, 3H), 2.65 (s, 3H). MS (ESI) m/z: 454.0 (M+1)⁺.

### Example 198. Synthesis of 3-(3-fluoro-4-methoxyphenyl)-8-(6-methylpyridin-3-yl)-2-(oxazol-5-yl)-4-oxo-3,4-dihydroquinazoline-6-carbonitrile (53c)

Prepared according to Scheme 10.

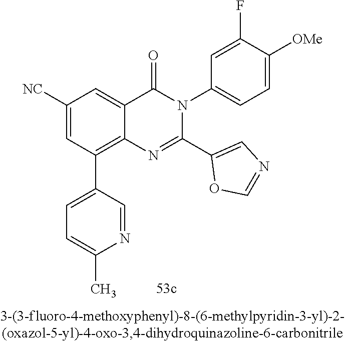 [SMILES: COc1ccc(-n2c(-c3cnco3)nc3c(-c4ccc(C)nc4)cc(C#N)cc3c2=O)cc1F]

### Synthesis of 8-bromo-3-(3-fluoro-4-methoxyphenyl)-2-(oxazol-5-yl)-4-oxo-3,4-dihydroquinazoline-6-carbonitrile (52c)

The procedure leading to 52b was followed using 51 (300 mg, 0.82 mmol), POCl₃ (8 mL), oxazole-5-carboxylic acid (112 mg, 0.99 mmol), and triethylamine (0.23 mL, 1.65 mmol). Purification by flash chromatography (0% to 50% to 100% A (DCM:EtOAc=1:1)/Hexane) yielded 52c (110 mg, 32%) as a brown solid. ¹H NMR (400 MHz, DMSO-d₆) δ 8.69 (d, J=17.0 Hz, 2H), 8.56 (s, 1H), 7.59-7.48 (m, 1H), 7.46-7.31 (m, 2H), 6.22 (s, 1H), 3.97 (s, 3H).

### Synthesis of 3-(3-fluoro-4-methoxyphenyl)-8-(6-methylpyridin-3-yl)-2-(oxazol-5-yl)-4-oxo-3,4-dihydroquinazoline-6-carbonitrile (53c)

A mixture of 52c (100 mg, 0.23 mmol), cesium carbonate (148 mg, 0.45 mmol), 2-methylpyridine-5-boronic acid (40 mg, 0.30 mmol) and tetrakis(tri-phenylphosphine) palladium (0) (20 mg, 0.017 mmol) in 1,4-dioxane (5 mL) and H₂O (1 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 40% A (DCM:MeOH=100:1)/Hexane) to give the product, which was treated with a palladium scavenger (SiliaMetS Thiol, 3×20 mg), stirred at 40° C. for 8 h and at rt for 16 h, and filtered through Celite pad to remove any residual metal from the coupling reaction. Repeating this procedure of removing Pd for three times. After concentration, 53c (40 mg, 32%) was obtained as a yellow solid. ¹H NMR (400 MHz, DMSO-d₆) δ 8.93 (s, 1H), 8.69-8.59 (m, 2H), 8.48 (s, 1H), 8.21-8.13 (m, 1H), 7.63-7.56 (m, 1H), 7.49 (d, J=8.3 Hz, 1H), 7.47-7.38 (m, 2H), 6.26 (s, 1H), 4.02 (s, 3H), 2.63 (s, 3H). MS (ESI) m/z: 454.0 (M+1)⁺.

### Example 199. Synthesis of 3-(3-fluoro-4-methoxyphenyl)-2-(isoxazol-3-yl)-8-(6-methylpyridin-3-yl)-4-oxo-3,4-dihydroquinazoline-6-carbonitrile (53d)

Prepared according to Scheme 10.

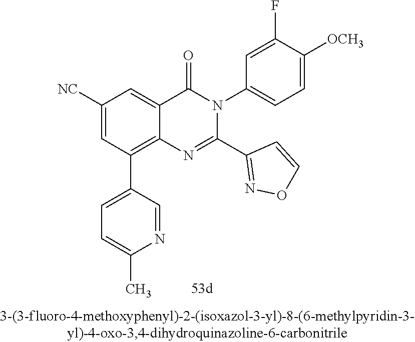 [SMILES: COc1ccc(-n2c(-c3ccon3)nc3c(-c4ccc(C)nc4)cc(C#N)cc3c2=O)cc1F]

### Synthesis of 8-bromo-3-(3-fluoro-4-methoxyphenyl)-2-(isoxazol-3-yl)-4-oxo-3,4-dihydroquinazoline-6-carbonitrile (52d)

The procedure leading to 52b was followed using 51 (400 mg, 1.10 mmol), POCl₃ (8 mL), isoxazole-3-carboxylic acid (149 mg, 1.32 mmol), and triethylamine (0.31 mL, 2.20 mmol). Purification by flash chromatography (0% to 40% A (DCM:EtOAc=1:1)/Hexane) yielded 52d (220 mg, 45%) as a brown solid. ¹H NMR (400 MHz, DMSO-d₆) δ 8.97 (s, 1H), 8.76 (s, 1H), 8.62 (s, 1H), 7.53-7.32 (m, 1H), 7.22 (s, 2H), 6.69 (s, 1H), 3.88 (s, 3H).

### Synthesis of 3-(3-fluoro-4-methoxyphenyl)-8-(6-methylpyridin-3-yl)-2-(oxazol-5-yl)-4-oxo-3,4-dihydroquinazoline-6-carbonitrile (53d)

A mixture of 52d (180 mg, 0.41 mmol), cesium carbonate (267 mg, 0.82 mmol), 2-methylpyridine-5-boronic acid (73 mg, 0.53 mmol) and tetrakis(tri-phenylphosphine) palladium (0) (36 mg, 0.031 mmol) in 1,4-dioxane (5 mL) and H₂O (1 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 50% A (DCM:MeOH=100:1)/Hexane) to give the product, which was treated with a palladium scavenger (SiliaMetS Thiol, 3×20 mg), stirred at 40° C. for 8 h and at rt for 16 h, and filtered through Celite pad to remove any residual metal from the coupling reaction. Repeating this procedure of removing Pd for three times. After concentration, 53d (60 mg, 32%) was obtained as a yellow solid. ¹H NMR (400 MHz, Chloroform-d) δ 8.82 (s, 1H), 8.68 (s, 1H), 8.33 (s, 1H), 8.06 (s, 1H), 7.98-7.81 (m, 1H), 7.39-7.29 (m, 1H), 7.03 (s, 3H), 6.58 (s, 1H), 3.95 (s, 3H), 2.66 (s, 3H). MS (ESI) m/z: 454.0 (M+1)⁺.

### Example 200. Synthesis of 3-(3-fluoro-4-methoxyphenyl)-2-(1-methyl-1H-imidazol-4-yl)-8-(6-methylpyridin-3-yl)-4-oxo-3,4-dihydroquinazoline-6-carbonitrile (53e)

Prepared according to Scheme 10.

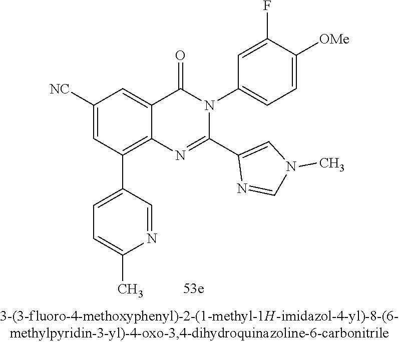 [SMILES: COc1ccc(-n2c(-c3cn(C)cn3)nc3c(-c4ccc(C)nc4)cc(C#N)cc3c2=O)cc1F]

### Synthesis of 8-bromo-3-(3-fluoro-4-methoxyphenyl)-2-(1-methyl-1H-imidazol-4-yl)-4-oxo-3,4-dihydroquinazoline-6-carbonitrile (52e)

The procedure leading to 52b was followed using 51 (400 mg, 1.10 mmol), POCl₃ (8 mL), 1-methyl-1H-imidazole-4-carboxylic acid (166 mg, 1.32 mmol), and triethylamine (0.31 mL, 2.20 mmol). Purification by flash chromatography (0% to 60% A (DCM:MeOH=100:1)/Hexane) yielded 52e (240 mg, 48%) as a yellow solid.

### Synthesis of 3-(3-fluoro-4-methoxyphenyl)-2-(1-methyl-1H-imidazol-4-yl)-8-(6-methylpyridin-3-yl)-4-oxo-3,4-dihydroquinazoline-6-carbonitrile (53e)

A mixture of 52e (300 mg, 0.66 mmol), cesium carbonate (431 mg, 1.32 mmol), 2-methylpyridine-5-boronic acid (118 mg, 0.86 mmol) and tetrakis(tri-phenylphosphine) palladium (0) (57 mg, 0.050 mmol) in 1,4-dioxane (10 mL) and H₂O (2 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 80% A (DCM:MeOH=100:1)/Hexane) to give the product, which was treated with a palladium scavenger (SiliaMetS Thiol, 3×30 mg), stirred at 40° C. for 8 h and at rt for 16 h, and filtered through Celite pad to remove any residual metal from the coupling reaction. Repeating this procedure of removing Pd for three times. After concentration, 53e (100 mg, 32%) was obtained as a yellow solid. ¹H NMR (400 MHz, DMSO-d₆) δ 8.83 (s, 1H), 8.49 (s, 1H), 8.29 (s, 1H), 8.16-7.98 (m, 1H), 7.54-7.26 (m, 4H), 7.23-7.03 (m, 2H), 3.89 (s, 3H), 3.60 (s, 3H), 2.57 (s, 3H). MS (ESI) m/z: 467.0 (M+1)⁺.

### Example 201. Synthesis of 3-(3-fluoro-4-methoxyphenyl)-2-(1-methyl-1H-imidazol-5-yl)-8-(6-methylpyridin-3-yl)-4-oxo-3,4-dihydroquinazoline-6-carbonitrile (53f)

Prepared according to Scheme 10.

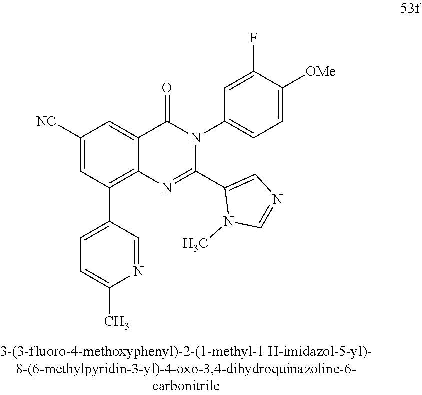 [SMILES: COc1ccc(-n2c(-c3cncn3C)nc3c(-c4ccc(C)nc4)cc(C#N)cc3c2=O)cc1F]

### Synthesis of 8-bromo-3-(3-fluoro-4-methoxyphenyl)-2-(1-methyl-1H-imidazol-5-yl)-4-oxo-3,4-dihydroquinazoline-6-carbonitrile (52f)

The procedure leading to 52b was followed using 51 (500 mg, 1.37 mmol), POCl₃ (8 mL), 1-methyl-1H-imidazole-5-carboxylic acid (208 mg, 1.65 mmol), and triethylamine (0.38 mL, 2.75 mmol). Purification by flash chromatography (0% to 80% A (DCM:MeOH=100:1)/Hexane) yielded 53f (130 mg, Y %=21%) as a yellow solid.

### Synthesis of 3-(3-fluoro-4-methoxyphenyl)-2-(1-methyl-1H-imidazol-5-yl)-8-(6-methylpyridin-3-yl)-4-oxo-3,4-dihydroquinazoline-6-carbonitrile (53f)

A mixture of 52f (120 mg, 0.27 mmol), cesium carbonate (173 mg, 0.53 mmol), 2-methylpyridine-5-boronic acid (47 mg, 0.34 mmol) and tetrakis(tri-phenylphosphine) palladium (0) (23 mg, 0.02 mmol) in 1,4-dioxane (5 mL) and H₂O (1 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×50 mL). The combined organic layers were washed with brine (100 mL), dried over anhydrous Na₂SO₄, and filtered, then the solvent was evaporated under reduced pressure to afford a pale-yellow oil. The crude product was purified with flash chromatography (0% to 90% A (DCM:MeOH=100:1)/Hexane) to give the product, which was treated with a palladium scavenger (SiliaMetS Thiol, 3×20 mg), stirred at 40° C. for 8 h and at rt for 16 h, and filtered through Celite pad to remove any residual metal from the coupling reaction. Repeating this procedure of removing Pd for three times. After concentration, 53f (60 mg, 49%) was obtained as a yellow solid. ¹H NMR (400 MHz, DMSO-d₆) δ 8.72 (d, J=2.3 Hz, 1H), 8.65 (d, J=2.0 Hz, 1H), 8.38 (d, J=2.0 Hz, 1H), 7.98 (dd, J=7.9, 2.3 Hz, 1H), 7.73 (s, 1H), 7.50 (d, 1H), 7.46 (d, J=8.0 Hz, 1H), 7.40-7.27 (m, 2H), 6.28 (s, 1H), 3.96 (s, 3H), 3.63 (s, 3H), 2.60 (s, 3H). MS (ESI) m/z: 467.0 (M+1)⁺.

### Example 202. Synthesis of 3-(3-fluoro-4-methoxyphenyl)-8-(6-methylpyridin-3-yl)-2-(oxazol-2-yl)-4-oxo-3,4-dihydroquinazoline-6-carbonitrile (55a)

Prepared according to Scheme 10.

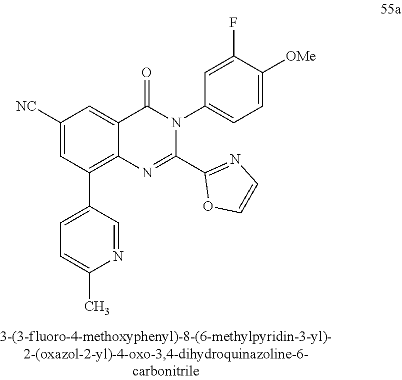 [SMILES: COc1ccc(-n2c(-c3ncco3)nc3c(-c4ccc(C)nc4)cc(C#N)cc3c2=O)cc1F]

### Synthesis of 2-amino-5-cyano-N-(3-fluoro-4-methoxyphenyl)-3-(6-methylpyridin-3-yl)benzamide (54)

A mixture of 51 (1 g, 2.75 mmol), cesium carbonate (1.79 g, 5.50 mmol), 2-methylpyridine-5-boronic acid (0.49 g, 3.57 mmol) and tetrakis(tri-phenylphosphine) palladium (0) (238 mg, 0.21 mmol) in 1,4-dioxane (60 mL) and H₂O (8 mL) was degassed and flushed with nitrogen. The mixture was heated at 85° C. for 16 h under nitrogen atmosphere. After cooling to room temperature, the mixture was concentrated to remove dioxane, and diluted with H₂O (50 mL) and extracted with DCM (3×100 mL). The combined organic layers were washed with brine (200 mL), dried over anhydrous Na₂SO₄, and filtered, then the solvent was evaporated under reduced pressure to afford a pale-yellow oil. The crude product was purified with flash chromatography (0% to 70% A (DCM:ethyl acetate=2:3)/Hexane) to give 54 (550 mg, 53%) as a grey solid. ¹H NMR (400 MHz, DMSO-d₆) δ 10.31 (s, 1H), 8.52-8.41 (m, 1H), 8.22-8.10 (m, 1H), 7.76-7.71 (m, 1H), 7.71-7.62 (m, 1H), 7.53 (s, 1H), 7.48-7.41 (m, 1H), 7.41-7.33 (m, 1H), 7.18 (t, J=9.3 Hz, 1H), 6.86 (s, 2H), 3.84 (s, 3H), 2.54 (s, 3H).

### Synthesis of 3-(3-fluoro-4-methoxyphenyl)-8-(6-methylpyridin-3-yl)-2-(oxazol-2-yl)-4-oxo-3,4-dihydroquinazoline-6-carbonitrile (55a)

To the mixture of 54 (270 mg, 0.72 mmol) in POCl₃ (8 mL) was added oxazole-2-carboxylic acid (97 mg, 0.86 mmol), and triethylamine (0.2 mL, 1.44 mmol) in a seal tube. The reaction mixture was heated to 150° C. for 1 h under microwave. Then, the reaction mixture was concentrated to give the resulting residual, which was adjusted to PH=10-11 using saturated aqueous Na₂CO₃. The aqueous layer was extracted with DCM (3×50 mL). The combined organic layers were washed with brine (100 mL), dried over anhydrous Na₂SO₄, and filtered, then the solvent was evaporated under reduced pressure to afford the crude product. It was purified by flash chromatography (0% to 75% A (DCM:MeOH=100:1)/Hexane) to give the product, which was treated with a palladium scavenger (SiliaMetS Thiol, 3×50 mg), stirred at 40° C. for 8 h and at rt for 16 h, and filtered through Celite pad to remove any residual metal from the coupling reaction. Repeating this procedure of removing Pd for three times. After concentration, 55a (130 mg, 40%) was obtained as a pale-yellow solid. ¹H NMR (400 MHz, DMSO-d₆) δ 8.85 (s, 1H), 8.63 (s, 1H), 8.46 (s, 1H), 8.25 (s, 1H), 8.08 (d, J=8.1 Hz, 1H), 7.50-7.37 (m, 2H), 7.31 (s, 1H), 7.22 (d, J=8.3 Hz, 2H), 3.89 (s, 3H), 2.56 (s, 3H). MS (ESI) m/z: 454.0 (M+1)⁺.

### Example 203. Synthesis of 3-(3-fluoro-4-methoxyphenyl)-2-(1-methyl-1H-imidazol-2-yl)-8-(6-methylpyridin-3-yl)-4-oxo-3,4-dihydroquinazoline-6-carbonitrile (55b)

Prepared according to Scheme 10.

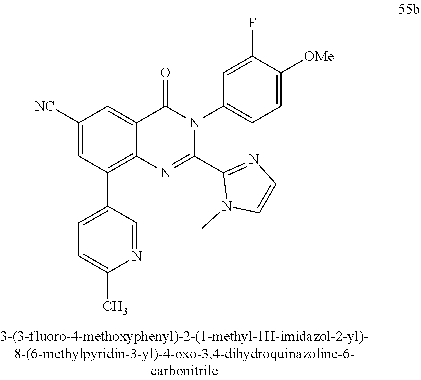 [SMILES: COc1ccc(-n2c(-c3nccn3C)nc3c(-c4ccc(C)nc4)cc(C#N)cc3c2=O)cc1F]

### Synthesis of 3-(3-fluoro-4-methoxyphenyl)-2-(1-methyl-1H-imidazol-2-yl)-8-(6-methylpyridin-3-yl)-4-oxo-3,4-dihydroquinazoline-6-carbonitrile (55b)

To the mixture of 54 (650 mg, 1.73 mmol) in POCl₃ (10 mL) was added 1-methyl-1H-imidazole-2-carboxylic acid (262 mg, 2.08 mmol), and triethylamine (0.48 mL, 3.45 mmol) in a sealed tube. The reaction mixture was heated to 180° C. for 1 h under microwave. Then, the reaction mixture was concentrated to give the resulting residual, which was adjusted to PH=10-11 using saturated aqueous Na₂CO₃. The aqueous layer was extracted with DCM (3×80 mL). The combined organic layers were washed with brine (200 mL), dried over anhydrous Na₂SO₄, and filtered, then the solvent was evaporated under reduced pressure to afford the crude product. It was purified by flash reverse phase (C-18) chromatography (0-30% CH₃CN/H₂O) to give the product, which was treated with a palladium scavenger (SiliaMetS Thiol, 3×50 mg), stirred at 40° C. for 8 h and at rt for 16 h, and filtered through Celite pad to remove any residual metal from the coupling reaction. Repeating this procedure of removing Pd for three times. After concentration, 55b (160 mg, 17%) was obtained as a pale-yellow solid. ¹H NMR (400 MHz, DMSO-d₆) δ 8.76 (s, 1H), 8.68 (s, 1H), 8.42 (s, 1H), 8.01 (dd, J=8.0, 2.3 Hz, 1H), 7.46 (d, J=8.0 Hz, 1H), 7.37 (d, J=12.0, 2.2 Hz, 1H), 7.23 (s, 1H), 7.21-7.09 (m, 2H), 6.83 (s, 1H), 3.89 (s, 3H), 3.77 (s, 3H), 2.59 (s, 3H). MS (ESI) m/z: 467.0 (M+1)⁺.

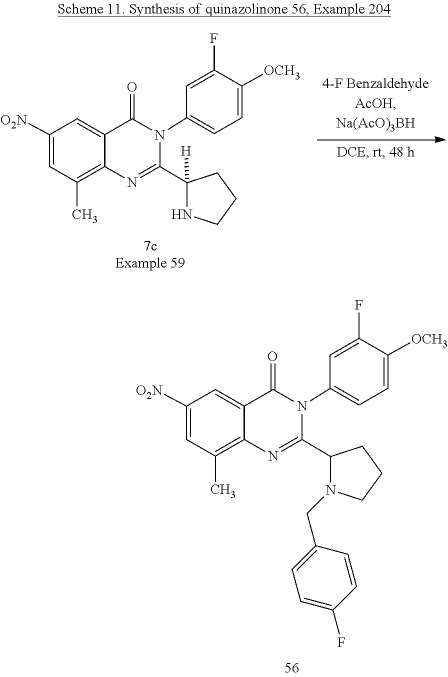 [SMILES: COc1ccc(-n2c([C@H]3CCCN3)nc3c(C)cc([N+](=O)[O-])cc3c2=O)cc1F ; COc1ccc(-n2c(C3CCCN3Cc3ccc(F)cc3)nc3c(C)cc([N+](=O)[O-])cc3c2=O)cc1F]

### Example 204. Synthesis of 3-(3-fluoro-4-methoxyphenyl)-2-(1-(4-fluorobenzyl)pyrrolidin-2-yl)-8-methyl-6-nitroquinazolin-4 (3H)-one (56)

Prepared according to Scheme 11.

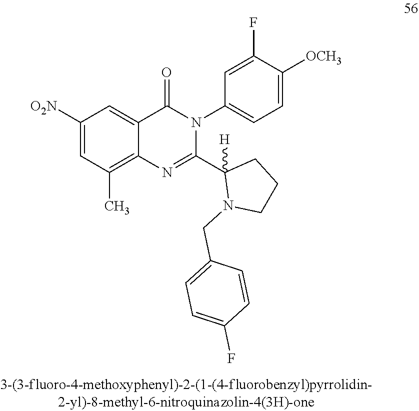 [SMILES: COc1ccc(-n2c(C3CCCN3Cc3ccc(F)cc3)nc3c(C)cc([N+](=O)[O-])cc3c2=O)cc1F]

3-(3-fluoro-4-methoxyphenyl)-2-(1-(4-fluorobenzyl)pyrrolidin-2-yl)-8-methyl-6-nitroquinazolin-4 (3H)-one (56): To a vial equipped with stir bar and DCE (2 mL) was added 4-fluorobenzaldehyde (0.05 mL, 0.47 mmol, 1.1 equiv.), (R)-3-(3-fluoro-4-methoxyphenyl)-8-methyl-6-nitro-2-(pyrrolidin-2-yl) quinazolin-4 (3H)-one (7c, Example 59) (174 mg, 0.44 mmol, 1.0 equiv.), sodium triacetoxyborohydride (102 mg, 0.48 mmol, 1.1 equiv.), and acetic acid (0.01 mL, 0.17 mmol, 0.39 equiv.). The reaction was stirred at rt for 25.5 h, and then 3 Å mol sieves were added as well as another equivalent sodium triacetoxyborohydride (96 mg, 0.45 mmol, 1.0 equiv) and the reaction stirred at rt for a further 16.5 h. The reaction was concentrated and purified by MPLC (4 g SiO₂, 20 mL/min, 0-50% EtOAc/Hexanes over 12 min) to give an orange oil, which was washed with Et₂O (2×10 mL) to give 56 as an orange foam (184 mg, 0.36 mmol, 83% yield; LCMS Purity ≥97%). ¹H NMR (400 MHz, CDCl₃) δ 8.93 (d, J=2.7 Hz, 1H), 8.43 (d, J=2.7 Hz, 1H), 7.16 (dt, J=9.0, 5.3 Hz, 2H), 7.04-6.82 (m, 4H), 6.42-6.20 (m, 1H), 3.95 (s, 3H), 3.90 (d, J=13.1 Hz, 1H), 3.67 (d, J=13.0 Hz, 1H), 3.35 (td, J=8.4, 4.9 Hz, 1H), 2.81 (dq, J=8.3, 4.6, 3.6 Hz, 1H), 2.75 (s, 3H), 2.17-1.82 (m, 4H).

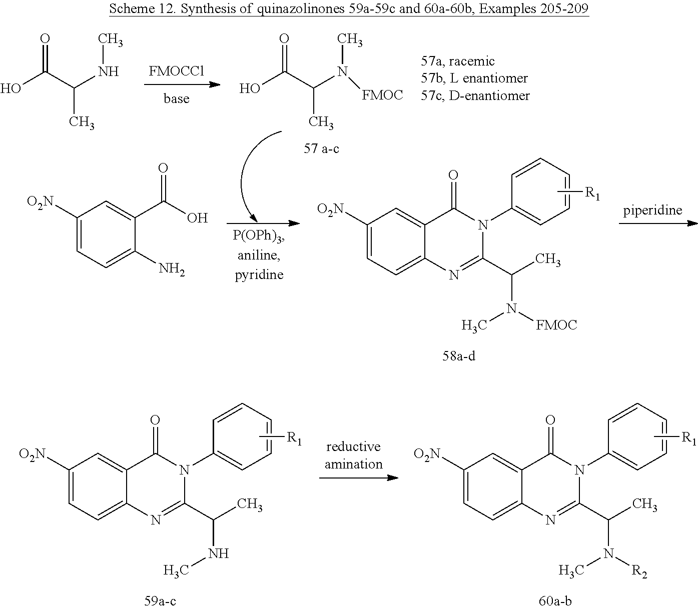

### Example 205. Synthesis of 3-(4-methoxyphenyl)-2-(1-(methylamino)ethyl)-6-nitroquinazolin-4 (3H)-one (59a)

Prepared according to Scheme 12.

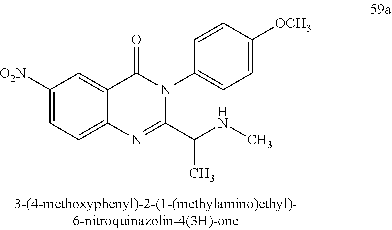 [SMILES: CNC(C)c1nc2ccc([N+](=O)[O-])cc2c(=O)n1-c1ccc(OC)cc1]

Step 1. Synthesis of N-(((9H-fluoren-9-yl)methoxy)carbonyl)-N-methylalanine (57a). To a solution of N-Methyl-DL-alanine (4.12 g, 40.00 mmol) in THF (200 mL) and 10% Na₂CO₃ (200 mL) was added 9-fluorenylmethyloxycarbonyl chloride (10.35 g, 40.00 mmol). The reaction was stirred at room temperature for 6 h, and then partitioned between ether (3×400 mL) and H₂O (600 mL). Then, the aqueous layer was adjusted to pH 2 with 1.00N HCl. The resulting suspension was extracted with EtOAc (4×300 mL). The combined organic layers were washed with water (300 mL) and brine (300 mL), dried over anhydrous Na₂SO₄, and filtered, and the solvent was evaporated under reduced pressure to afford N-(((9H-fluoren-9-yl)methoxy)carbonyl)-N-methylalanine 57a (9.75 g, 74.9%) as off-white solid. ¹H NMR (400 MHz, CDCl₃) δ 7.69-7.65 (m, 2H), 7.53-7.45 (m, 2H), 7.34-7.30 (m, 2H), 7.23-7.17 (m, 2H), 4.85-4.54 (m, 1H), 4.43-4.30 (m, 2H), 4.21-4.15 (m, 1H), 2.87-2.80 (m, 3H), 1.38 (d, J=7.3 Hz, 2H), 1.29 (d, J=7.1 Hz, 1H). MS (ESI) m/z: 326 (M+1)⁺.

Step 2. Synthesis of (9H-fluoren-9-yl)methyl(1-(3-(4-methoxyphenyl)-6-nitro-4-oxo-3,4-dihydroquinazolin-2-yl)ethyl)(methyl)carbamate (58a). 5-Nitroanthranilic acid (1.47 g, 8.00 mmol) and N-(((9H-fluoren-9-yl)methoxy)carbonyl)-N-methylalanine 57a (5.20 g, 16.00 mmol) were dissolved in dry pyridine (48.00 mL). Triphenyl phosphite (6.24 mL, 24.00 mmol) was added to the mixture and the reaction mixture was heated at 100° C. with MWI for 2×20 min. 4-methoxyaniline (3.92 g, 32.00 mmol) was added to the mixture and the reaction mixture was heated at 100° C. with MWI for 1 min. Pyridine was removed in vacuo and the residue was diluted with CH₂Cl₂ (300 mL) and washed with 1N HCl aqueous (200 mL). The organic layer was washed with H₂O (200 mL) and brine (200 mL), dried over anhydrous Na₂SO₄, and filtered, then the solvent was evaporated under reduced pressure. The residue was purified by flash chromatography (0% ethyl acetate/CH₂Cl₂ to 5% ethyl acetate/CH₂Cl₂) to afford (9H-fluoren-9-yl)methyl(1-(3-(4-methoxyphenyl)-6-nitro-4-oxo-3,4-dihydroquinazolin-2-yl)ethyl)(methyl)carbamate 58a (1.86 g, 40.3%) as pale yellow solid. ¹H NMR (400 MHz, CDCl₃) δ 9.11 (dd, J=5.9, 2.4 Hz, 1H), 8.60-8.52 (m, 1H), 7.89-7.81 (m, 1H), 7.79-7.66 (m, 2H), 7.58 (t, J=6.7 Hz, 1H), 7.42 (dq, J=14.8, 7.4 Hz, 2H), 7.30 (dd, J=9.7, 5.1 Hz, 2H), 7.23 (dd, J=12.5, 10.2 Hz, 1H), 7.18-7.10 (m, 1H), 7.03 (dd, J=14.1, 5.4 Hz, 1H), 6.98-6.76 (m, 2H), 5.07 (td, J=14.0, 7.2 Hz, 1H), 4.32-4.16 (m, 2H), 4.02-3.89 (m, 1H), 3.74 (d, J=6.1 Hz, 3H), 2.93 (s, 2H), 2.81 (s, 1H), 1.43 (t, J=6.4 Hz, 2H), 1.25 (s, 1H). MS (ESI) m/z: 577 (M+1)⁺.

Step 3. Synthesis of 3-(4-methoxyphenyl)-2-(1-(methylamino)ethyl)-6-nitroquinazolin-4 (3H)-one (59a). To a solution of (9H-fluoren-9-yl)methyl(1-(3-(4-methoxyphenyl)-6-nitro-4-oxo-3,4-dihydroquinazolin-2-yl)ethyl)(methyl)carbamate 58a (1.86 g, 3.24 mmol) in CH₂Cl₂ (30 mL) was added piperidine (3.24 mL, 32.40 mmol). The resulting mixture was stirred at room temperature for 3 h. The solvent was evaporated under reduced pressure. The residue was purified by flash chromatography (0% MeOH/Ethyl acetate-10% MeOH/Ethyl acetate) to afford 59a (0.76 g, 66.2%) as pale yellow solid. ¹H NMR (500 MHz, CDCl₃) δ 9.12 (d, J=2.6 Hz, 1H), 8.55 (dd, J=9.0, 2.6 Hz, 1H), 7.83 (d, J=9.0 Hz, 1H), 7.20-7.15 (m, 2H), 7.09 (ddd, J=9.7, 7.8, 2.1 Hz, 2H), 3.90 (s, 3H), 3.44 (q, J=6.6 Hz, 1H), 2.29 (s, 3H), 1.27 (d, J=6.6 Hz, 3H). MS (ESI) m/z: 355.1 (M+1)⁺.

### Example 206. Synthesis of (S)-3-(4-methoxyphenyl)-2-(1-(methylamino)ethyl)-6-nitroquinazolin-4 (3H)-one (59b)

Prepared according to Scheme 12.

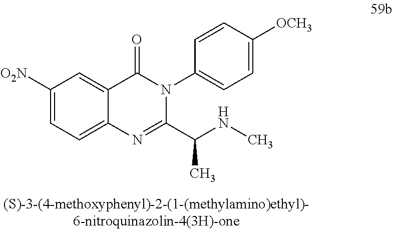 [SMILES: CN[C@@H](C)c1nc2ccc([N+](=O)[O-])cc2c(=O)n1-c1ccc(OC)cc1]

Step 1. Synthesis of (S)—N-(((9H-fluoren-9-yl) methoxy) carbonyl)-N-methylalanine (57b). To a solution of N-methyl-L-alanine hydrochloride (1.39 g, 10.00 mmol) in THE (50 mL) and 10% Na₂CO₃ (50 mL) was added 9-fluorenylmethyloxycarbonyl chloride (2.59 g, 10.00 mmol). The reaction was stirred at room temperature for 6 h, and then partitioned between ether (3×200 mL) and H₂O (400 mL). Then, the aqueous layer was adjusted to pH 2 with 1.00N HCl. The resulting suspension was extracted with EtOAc (3×200 mL). The combined organic layers were washed with water (200 mL) and brine (200 mL), dried over anhydrous Na₂SO₄, and filtered, and the solvent was evaporated under reduced pressure to afford 57b (2.65 g, 81.4%) as off-white solid. ¹H NMR (400 MHz, CDCl₃) δ 7.71-7.68 (m, 2H), 7.55-7.42 (m, 2H), 7.36-7.31 (m, 2H), 7.24-7.18 (m, 2H), 4.86-4.51 (m, 1H), 4.45-4.31 (m, 2H), 4.25-4.16 (m, 1H), 2.86-2.84 (m, 3H), 1.36 (d, J=7.3 Hz, 2H), 1.28 (d, J=7.1 Hz, 1H). MS (ESI) m/z: 326 (M+1)⁺.

Step 2. Synthesis of (9H-fluoren-9-yl)methyl (S)-(1-(3-(4-methoxyphenyl)-6-nitro-4-oxo-3,4-dihydroquinaz-ol-in-2-yl)ethyl)(methyl)carbamate (58b). 5-nitroanthranilic acid (183.13 mg, 1.00 mmol) and 57b (0.65 g, 2.00 mmol) were dissolved in dry pyridine (6 mL). Triphenyl phosphite (0.79 mL, 3.00 mmol) was added to the mixture and the reaction mixture was heated at 100° C. with MWI for 2×20 min. 4-methoxyaniline (0.50 g, 4.00 mmol) was added to the mixture and the reaction mixture was heated at 100° C. with MWI for 1 min. Pyridine was removed in vacuo and the residue was diluted with CH₂Cl₂ (100 mL) and washed with 1N HCl aqueous (40 mL). The organic layer was washed with H₂O (40 mL) and brine (40 mL), dried over anhydrous Na₂SO₄, and filtered, then the solvent was evaporated under reduced pressure. The residue was purified by flash chromatography (0% ethyl acetate/CH₂Cl₂ to 5% ethyl acetate/CH₂Cl₂) to afford 58b (0.23 g, 39.9%) as pale yellow solid. ¹H NMR (400 MHz, CDCl₃) δ 9.08 (dd, J=5.6, 2.4 Hz, 1H), 8.65-8.51 (m, 1H), 7.86-7.78 (m, 1H), 7.78-7.68 (m, 2H), 7.61 (t, J=6.6 Hz, 1H), 7.41 (dq, J=14.8, 7.4 Hz, 2H), 7.34 (dd, J=9.7, 5.1 Hz, 2H), 7.25 (dd, J=12.5, 10.2 Hz, 1H), 7.21-7.08 (m, 1H), 7.04 (dd, J=14.1, 5.4 Hz, 1H), 6.91-6.78 (m, 2H), 5.05 (td, J=14.0, 7.2 Hz, 1H), 4.31-4.14 (m, 2H), 4.06-3.85 (m, 1H), 3.71 (d, J=6.1 Hz, 3H), 2.95 (s, 2H), 2.84 (s, 1H), 1.45 (t, J=6.4 Hz, 2H), 1.28 (s, 1H). MS (ESI) m/z: 577 (M+1)⁺.

Step 3. Synthesis of (S)-3-(4-methoxyphenyl)-2-(1-(methylamino)ethyl)-6-nitroquinazolin-4 (3H)-one (59b). To a solution of compound 58b (0.11 g, 0.19 mmol) in CH₂Cl₂ (3 mL) was added piperidine (0.19 mL, 1.91 mmol). The resulting mixture was stirred at room temperature for 3 h. The solvent was evaporated under reduced pressure. The residue was purified by flash chromatography (0% MeOH/CH₂Cl₂-10% MeOH/CH₂Cl₂) to afford 59b (42.68 mg, 63.1%) as pale yellow solid. ¹H NMR (500 MHz, CDCl₃) δ 9.10 (d, J=2.0 Hz, 1H), 8.54 (dd, J=8.9, 2.1 Hz, 1H), 7.83 (d, J=8.9 Hz, 1H), 7.17 (t, J=6.8 Hz, 2H), 7.09 (t, J=6.9 Hz, 3H), 3.90 (s, 3H), 3.44 (dd, J=13.0, 6.4 Hz, 1H), 2.29 (s, 3H), 1.28 (d, J=6.5 Hz, 3H). MS (ESI) m/z: 355.1 (M+1)⁺.

### Example 207. Synthesis of (R)-3-(4-methoxyphenyl)-2-(1-(methylamino)ethyl)-6-nitroquinazolin-4 (3H)-one (59c)

Prepared according to Scheme 12.

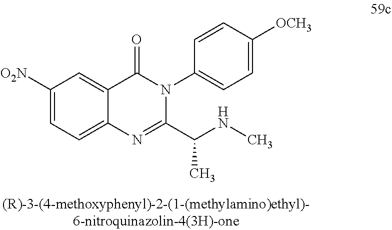 [SMILES: CN[C@H](C)c1nc2ccc([N+](=O)[O-])cc2c(=O)n1-c1ccc(OC)cc1]

Step 1. Synthesis of (R)—N-(((9H-fluoren-9-yl) methoxy) carbonyl)-N-methylalanine (57c). To a solution of N-methyl-D-alanine (1.03 g, 10.00 mmol) in THE (50 mL) and 10% Na₂CO₃ (50 mL) was added 9-Fluorenylmethyloxycarbonyl chloride (2.59 g, 10.00 mmol). The reaction was stirred at room temperature for 6 h, and then partitioned between ether (3×200 mL) and H₂O (400 mL). Then, the aqueous layer was adjusted to pH 2 with 1.00N HCl. The resulting suspension was extracted with EtOAc (3×200 mL). The combined organic layers were washed with water (200 mL) and brine (200 mL), dried over anhydrous Na₂SO₄, and filtered, and the solvent was evaporated under reduced pressure to afford 57c (3.03 g, 93.1%) as off-white solid. ¹H NMR (400 MHz, CDCl₃) δ 7.71-7.66 (m, 2H), 7.51-7.42 (m, 2H), 7.38-7.31 (m, 2H), 7.25-7.16 (m, 2H), 4.86-4.51 (m, 1H), 4.46-4.34 (m, 2H), 4.28-4.16 (m, 1H), 2.88-2.84 (m, 3H), 1.35 (d, J=7.3 Hz, 2H), 1.28 (d, J=7.1 Hz, 1H). MS (ESI) m/z: 326 (M+1)⁺.

Step 2. Synthesis of (9H-fluoren-9-yl)methyl (R)-(1-(3-(4-methoxyphenyl)-6-nitro-4-oxo-3,4-dihydroquinaz-ol-in-2-yl)ethyl)(methyl)carbamate (58c). 5-nitroanthranilic acid (183.13 mg, 1.00 mmol) and 57c (0.65 g, 2.00 mmol) were dissolved in dry pyridine (6 mL). Triphenyl phosphite (0.79 mL, 3.00 mmol) was added to the mixture and the reaction mixture was heated at 100° C. with MWI for 2×20 min. 4-methoxyaniline (0.50 g, 4.00 mmol) was added to the mixture and the reaction mixture was heated at 100° C. with MWI for 1 min. Pyridine was removed in vacuo and the residue was diluted with CH₂Cl₂ (100 mL) and washed with 1N HCl aqueous (40 mL). The organic layer was washed with H₂O (40 mL) and brine (40 mL), dried over anhydrous Na₂SO₄, and filtered, then the solvent was evaporated under reduced pressure. The residue was purified by flash chromatography (0% ethyl acetate/CH₂Cl₂ to 5% ethyl acetate/CH₂Cl₂) to afford 58c (0.18 g, 31.4%) as pale yellow solid. ¹H NMR (400 MHz, CDCl₃) δ 9.12 (dd, J=5.8, 2.4 Hz, 1H), 8.61-8.54 (m, 1H), 7.86-7.76 (m, 1H), 7.76-7.61 (m, 2H), 7.64 (t, J=6.6 Hz, 1H), 7.42 (dq, J=14.8, 7.6 Hz, 2H), 7.36 (dd, J=9.6, 5.1 Hz, 2H), 7.28 (dd, J=12.5, 10.2 Hz, 1H), 7.24-7.06 (m, 1H), 7.06 (dd, J=14.1, 5.4 Hz, 1H), 6.96-6.78 (m, 2H), 5.06 (td, J=14.0, 7.2 Hz, 1H), 4.34-4.15 (m, 2H), 4.06-3.81 (m, 1H), 3.72 (d, J=6.1 Hz, 3H), 2.96 (s, 2H), 2.86 (s, 1H), 1.48 (t, J=6.4 Hz, 2H), 1.25 (s, 1H). MS (ESI) m/z: 577 (M+1)⁺.

Step 3. Synthesis of (R)-3-(4-methoxyphenyl)-2-(1-(methylamino)ethyl)-6-nitroquinazolin-4 (3H)-one (59c). To a solution of compound 58c (100.00 mg, 0.17 mmol) in CH₂Cl₂ (3 mL) was added piperidine (0.17 mL, 1.73 mmol). The resulting mixture was stirred at room temperature for 3 h. The solvent was evaporated under reduced pressure. The residue was purified by flash chromatography (0% MeOH/CH₂Cl₂ to 10% MeOH/CH₂Cl₂) to afford 59c (37.05 mg, 60.3%) as pale yellow solid. ¹H NMR (500 MHz, CDCl₃) δ 9.10 (d, J=2.0 Hz, 1H), 8.54 (dd, J=8.9, 2.1 Hz, 1H), 7.83 (d, J=8.9 Hz, 1H), 7.17 (t, J=6.8 Hz, 2H), 7.09 (t, J=6.9 Hz, 3H), 3.90 (s, 3H), 3.44 (dd, J=13.0, 6.4 Hz, 1H), 2.29 (s, 3H), 1.28 (d, J=6.5 Hz, 3H). MS (ESI) m/z: 355.1 (M+1)⁺.

### Example 208. Synthesis of (S)-2-(1-(dimethylamino)ethyl)-3-(4-methoxyphenyl)-6-nitroquinazolin-4 (3H)-one (60a)

Prepared as shown in Scheme 12.

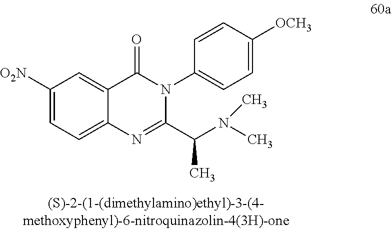 [SMILES: COc1ccc(-n2c([C@H](C)N(C)C)nc3ccc([N+](=O)[O-])cc3c2=O)cc1]

### Synthesis of(S)-2-(1-(dimethylamino)ethyl)-3-(4-methoxyphenyl)-6-nitroquinazolin-4 (3H)-one (60a)

Acetic acid (0.01 mL) was added to the solution of 59b (40.00 mg, 0.11 mmol) and formaldehyde solution 37.0% in H₂O (17.86 mg, 0.22 mmol) in MeOH (1.5 mL) and CH₂Cl₂ (1.5 mL), stirred at room temperature for 1 h, then sodium cyanoborohydride (10.36 mg, 0.16 mmol) was added to the reaction mixture at 0° C., then stirred at room temperature overnight, then the solvent was removed in vacuo and the residue was diluted with CH₂Cl₂ (20 mL) and washed with saturated solutions of Na₂CO₃ (15 mL) and brine (15 mL), dried over anhydrous Na₂SO₄, and filtered, then the solvent was evaporated under reduced pressure. The residue was purified by flash chromatography (0% MeOH/CH₂Cl₂ to 5% MeOH/CH₂Cl₂) to afford 60a (6.00 mg, 14.8%) as pale yellow solid. ¹H NMR (500 MHz, CDCl₃) δ 9.12 (d, J=2.5 Hz, 1H), 8.54 (dd, J=8.9, 2.5 Hz, 1H), 7.90 (d, J=9.0 Hz, 1H), 7.29 (dd, J=8.7, 2.4 Hz, 1H), 7.11-7.01 (m, 3H), 3.89 (s, 3H), 3.45 (dd, J=13.2, 6.6 Hz, 1H), 2.14 (s, 6H), 1.30 (d, J=6.6 Hz, 3H). MS (ESI) m/z: 369.1 (M+1)⁺.

### Example 209. Synthesis of (R)-2-(1-(dimethylamino)ethyl)-3-(4-methoxyphenyl)-6-nitroquinazolin-4 (3H)-one (60b)

Prepared as shown in Scheme 12.

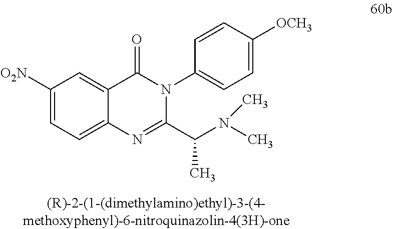 [SMILES: COc1ccc(-n2c([C@@H](C)N(C)C)nc3ccc([N+](=O)[O-])cc3c2=O)cc1]

### Synthesis of (R)-2-(1-(dimethylamino)ethyl)-3-(4-methoxyphenyl)-6-nitroquinazolin-4 (3H)-one (60b)

Acetic acid (0.01 mL) was added to the solution of 59c (40.00 mg, 0.11 mmol) and formaldehyde solution 37.0% in H₂O (17.86 mg, 0.22 mmol) in MeOH (1.5 mL) and CH₂Cl₂ (1.5 mL), stirred at room temperature for 1 h, then sodium cyanoborohydride (10.36 mg, 0.16 mmol) was added to the reaction mixture at 0° C., then stirred at room temperature overnight, then the solvent was removed in vacuo and the residue was diluted with CH₂Cl₂ (20 mL) and washed with saturated solutions of Na₂CO₃ (15 mL) and brine (15 mL), dried over anhydrous Na₂SO₄, and filtered, then the solvent was evaporated under reduced pressure. The residue was purified by flash chromatography (0% MeOH/CH₂Cl₂ to 5% MeOH/CH₂Cl₂) to afford 60b (8.90 mg, 22.0%) as pale yellow solid. ¹H NMR (500 MHz, CDCl₃) δ 9.12 (d, J=2.5 Hz, 1H), 8.54 (dd, J=8.9, 2.5 Hz, 1H), 7.90 (d, J=8.9 Hz, 1H), 7.29 (dd, J=8.6, 2.4 Hz, 1H), 7.11-7.06 (m, 2H), 7.04 (dd, J=8.5, 2.7 Hz, 1H), 3.89 (s, 3H), 3.45 (dd, J=13.2, 6.6 Hz, 1H), 2.14 (s, 6H), 1.30 (d, J=6.6 Hz, 3H). MS (ESI) m/z: 369.1 (M+1)⁺.

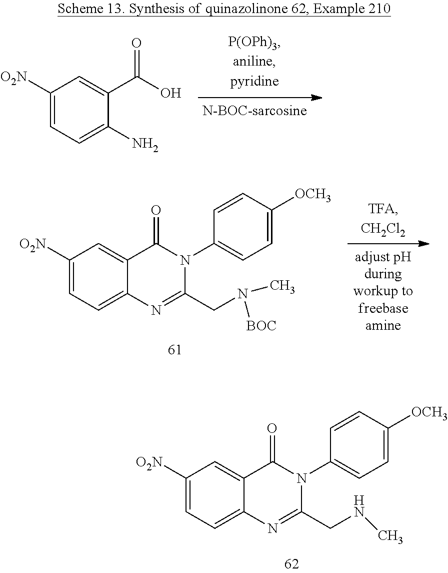 [SMILES: Nc1ccc([N+](=O)[O-])cc1C(=O)O ; COc1ccc(-n2c(CN(C)C(=O)OC(C)(C)C)nc3ccc([N+](=O)[O-])cc3c2=O)cc1 ; CNCc1nc2ccc([N+](=O)[O-])cc2c(=O)n1-c1ccc(OC)cc1]

### Example 210. Synthesis of 3-(4-methoxyphenyl)-2-((methylamino)methyl)-6-nitroquinazolin-4 (3H)-one (62)

Prepared according to Scheme 13.

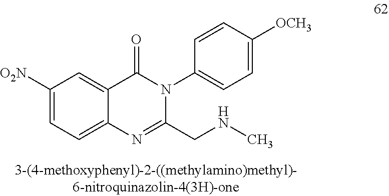 [SMILES: CNCc1nc2ccc([N+](=O)[O-])cc2c(=O)n1-c1ccc(OC)cc1]

Step 1. Synthesis of tert-butyl ((3-(4-methoxyphenyl)-6-nitro-4-oxo-3,4-dihydroquinazolin-2-yl)methyl)(methyl) carbamate (61). 5-nitroanthranilic acid (182.13 mg, 1.00 mmol) and N-Boc-sarcosine (378.42 mg, 2.00 mmol) were dissolved in dry pyridine (4 mL). Triphenyl phosphite (0.78 mL, 3.00 mmol) was added to the mixture and the reaction mixture was heated at 100° C. with MWI for 40 min. 4-methoxyaniline (0.49 g, 4.00 mmol) was added to the mixture and the reaction mixture was heated at 100° C. with MWI for 1 min. Pyridine was removed in vacuo and the residue was diluted with CH₂Cl₂ (160 mL) and washed with 1N aqueous HCl (60 mL). The organic layer was washed with H₂O (60 mL) and brine (60 mL), dried over anhydrous Na₂SO₄, and filtered, then the solvent was evaporated under reduced pressure. The residue was purified by flash chromatography (0% ethyl acetate/CH₂Cl₂ to 10% ethyl acetate/CH₂Cl₂) to afford 61 (0.13 g, 29.6%) as pale yellow solid. ¹H NMR (400 MHz, CDCl₃) δ 9.12 (d, J=4.2 Hz, 1H), 8.54 (t, J=9.2 Hz, 1H), 7.81 (dd, J=16.3, 8.9 Hz, 1H), 7.21 (d, J=8.7 Hz, 2H), 7.17-6.61 (m, 2H), 4.07 (d, J=26.1 Hz, 2H), 3.89 (d, J=7.3 Hz, 3H), 2.97 (s, 3H), 1.49 (s, 5H), 1.34 (s, 4H). MS (ESI) m/z: 441.1 (M+1)⁺.

Step 2. Synthesis of 3-(4-methoxyphenyl)-2-((methylamino)methyl)-6-nitroquinazolin-4 (3H)-one (62)

Under nitrogen, trifluoroacetic acid (0.26 mL, 3.60 mmol) was added to a solution of 61 (80.00 mg, 0.18 mmol) in dry CH₂Cl₂ (4 mL) at 0° C., then stirred at rt overnight, then the solvent was removed in vacuo and the residue was diluted with CH₂Cl₂ (20 mL) and washed with saturated solutions of Na₂CO₃ (10 mL) and brine (10 mL), dried over anhydrous Na₂SO₄, and filtered, then the solvent was evaporated under reduced pressure. The residue was purified by flash chromatography (0% MeOH/CH₂Cl₂ to 5% MeOH/CH₂Cl₂) to afford 62 (25.60 mg, 41.8%) as pale yellow amorphous solid. ¹H NMR (500 MHz, CDCl₃) δ 9.11 (d, J=2.6 Hz, 1H), 8.54 (dd, J=9.0, 2.7 Hz, 1H), 7.85 (d, J=9.0 Hz, 1H), 7.20-7.15 (m, 2H), 7.11-7.06 (m, 2H), 3.90 (s, 3H), 3.47 (s, 2H), 2.45 (s, 3H). MS (ESI) m/z: 341.1 (M+1)⁺.

### ACRONYMS/ABBREVIATIONS

VEEV=venezuelan equine encephalitis virus
WEEV=western equine encephalitis virus
EEEV=eastern equine encephalitis virus
TrD=Trinidad Donkey strain of VEEV
BID=twice daily
IP=intraperitoneal (abdominal cavity)

### In Vitro Assays:

VEEV strains of TC-83 (gift of Dr. Connie Schmaljohn, USAMRIID) and Trinidad Donkey (BEI Catalog No. NR-332), WEEV strain of California, and EEEV strains of FL93-939 (BEI Catalog No. NR-4156), BeAr436087 (BEI Catalog No. NR-41568), Crow #9464 (BEI Catalog No. NR-41509) were used for this study. Anti-viral activity of compounds was measured using a cell-based CPE assay as previously described. Briefly, Vero 76 cells were maintained in a Complete medium (Modified Eagle's Medium with Earle's Balanced Salt Solution supplemented with 1× GlutaMAX, and 10% FBS). Vero 76 cells seeded in a 96 well plate were infected with a virus at an MOI of 0.05 diluted in a virus infection media (VIM: Modified Eagle's Medium with Earle's Balanced Salt Solution supplemented with 1× GlutaMAX, 25 mM HEPES, and 10% FBS) in the presence of test compounds serially diluted to 8 different concentrations. Infected cells were incubated for 48 hours at 37° C. in an incubator with 5% CO₂ and cell viability, protection from virus-induced CPE, was measured using CellTiter-Glo (Promega). EC₅₀ was calculated with a 4-parameter logistic model (XLfit, IBDS). Each experiment were performed with at least of three replicates of cell plates.

TABLE 1
In vitro antiviral cytopathic effect (CPE) data for VEEV
TC83, WEEV California and EEEV
(strains FL93-939 and BeAr 436087) alphaviruses.

| | | | WEEV | EEEV | EEEV |
|---|---|---|---|---|---|
| | | VEEV | California | FL93- | BeAr |
| | | TC83 | EC₅₀ | 939 | 436087 |
| Example | Cmpd | EC₅₀ nM | nM | EC₅₀ | EC₅₀ nM |
| 1 | 2a | XXXX | XXXX | ND | ND |
| 2 | 2b | XXXXX | XXXX | ND | ND |
| 3 | 3a | XX | X | ND | ND |
| 4 | 3b | XXX | X | ND | ND |
| 5 | 2c | XXX | XXX | ND | ND |
| 6 | 3c | XX | X | ND | ND |
| 7 | 3d | X | X | ND | ND |
| 8 | 2d | XXXXX | XXXXX | ND | ND |
| 9 | 2e | XXXXX | XXXXX | ND | ND |
| 10 | 3e | XXXXX | XXX | XXX | XXXX |
| 11 | 3f | XXXX | XX | ND | ND |
| 12 | 2f | XXXXX | XXX | ND | ND |
| 13 | 3g | XXXXX | XXXX | ND | ND |
| 14 | 2g | XXXXX | XXXX | ND | ND |
| 15 | 2h | XXXX | XX | ND | ND |
| 16 | 2i | XX | X | ND | ND |
| 17 | 2j | XXX | X | ND | ND |
| 18 | 2k | XXXXX | XXX | XXX | XXXX |
| 19 | 2l | XXXXX | XXX | ND | ND |
| 20 | 2m | XXXXX | XXXX | ND | ND |
| 21 | 2n | X | X | ND | ND |
| 22 | 2o | X | X | ND | ND |
| 23 | 2p | XXXXX | XXXXX | ND | ND |
| 24 | 2q | XXXXX | XXXXX | ND | ND |
| 25 | 2r | XXXX | XXX | ND | ND |
| 26 | 2s | X | X | ND | ND |
| 27 | 2t | XX | X | ND | ND |
| 28 | 2u | XXX | X | ND | ND |
| 29 | 3h | X | X | ND | ND |
| 30 | 2v | X | X | ND | ND |
| 31 | 3i | X | X | ND | ND |
| 32 | 2w | X | X | ND | ND |
| 33 | 2x | XXX | XXX | ND | ND |
| 34 | 2y | XXXX | XXXX | ND | ND |
| 35 | 2z | X | X | ND | ND |
| 36 | 2aa | XXXX | XX | ND | ND |
| 37 | 2bb | XXXXX | XX | ND | ND |
| 38 | 2cc | X | X | ND | ND |
| 39 | 2dd | XXXX | X | ND | ND |
| 40 | 2ee | XXXXX | XX | ND | ND |
| 41 | 2ff | XX | X | ND | ND |
| 42 | 2gg | XXXX | XXX | ND | ND |
| 43 | 2hh | XXXXX | XXXXX | ND | ND |
| 44 | 2ii | X | X | ND | ND |
| 45 | 2jj | X | X | ND | ND |
| 46 | 2kk | X | X | ND | ND |
| 47 | 2ll | X | X | ND | ND |
| 48 | 2mm | X | X | ND | ND |
| 49 | 2nn | X | X | ND | ND |
| 50 | 5a | XXXXX | XXXXX | ND | ND |
| 51 | 5b | XXX | XX | ND | ND |
| 52 | 5c | XXXXX | XXXXX | ND | ND |
| 53 | 5d | XXX | XXX | ND | ND |
| 54 | 5e | XXX | XXX | ND | ND |
| 55 | 7a | XXXXX | XXXXX | XXXX | XXXX |
| | | | | | X |
| 56 | 7b | XXXXX | XXXX | ND | ND |
| 57 | 8a | XXXX | XXX | XXXX | XXXX |
| 58 | 8b | XXXXX | XXXXX | XXXX | XXXX |
| 59 | 7c | XXXXX | XXXXX | XXXX | XXXX |
| | | | | X | X |
| 60 | 7d | XXXXX | XXXXX | ND | ND |
| 61 | 7e | XXXXX | XXXX | XXXX | XXXX |
| 62 | 7f | XXXX | XXXX | ND | ND |
| 63 | 7g | X | X | ND | ND |
| 64 | 7h | X | X | ND | ND |
| 65 | 7i | XXXX | XXXX | ND | ND |
| 66 | 8c | XXX | XXX | ND | ND |
| 67 | 7j | XXX | XXX | ND | ND |
| 68 | 8d | XXX | XXX | ND | ND |
| 69 | 7k | XXX | XXXX | ND | ND |
| 70 | 8e | XXXXX | XXXXX | ND | ND |
| 71 | 8f | XXXX | XXXX | ND | ND |
| 72 | 7i | XXXXX | XXXX | ND | ND |
| 73 | 8c | X | X | ND | ND |
| 74 | 7j | XXXXX | XXXXX | ND | ND |
| 75 | 14b | XXXX | XXXX | ND | ND |
| 76 | 14c | XXXXX | XXXXX | ND | ND |
| 77 | 14d | XXXX | XXXXX | ND | ND |
| 78 | 17a | XXXX | XXXX | ND | ND |
| 79 | 17b | XX | XX | ND | ND |
| 80 | 19a | XXXXX | XXXXX | ND | ND |
| 81 | 21a | XXXXX | XXXXX | ND | ND |
| 82 | 21b | XXXXX | XXXXX | ND | ND |
| 83 | 21c | XXXXX | XXXXX | ND | ND |
| 84 | 21d | XXXXX | XXXXX | ND | ND |
| 85 | 21e | XXXXX | XXXXX | ND | ND |
| 86 | 21f | XXXXX | XXXXX | XXXX | XXXX |
| | | | | X | X |
| 87 | 21g | XXXXX | XXXXX | ND | ND |
| 88 | 21h | XXXXX | XXXXX | ND | ND |
| 89 | 21i | XXXXX | XXXXX | XXXX | XXXX |
| 90 | 21j | XXXXX | XXXXX | ND | ND |
| 91 | 21k | XXXXX | XXXXX | ND | ND |
| 92 | 21l | XXXXX | XXXXX | ND | ND |
| 93 | 21m | XXXXX | XXXXX | ND | ND |
| 94 | 21n | XXXXX | XXXX | ND | ND |
| 95 | 21o | XXXXX | XXXX | ND | ND |
| 96 | 23a | XXXXX | XXXXX | ND | ND |
| 97 | 23b | XXXXX | XXXXX | ND | ND |
| 98 | 24 | XXXXX | XXXXX | ND | ND |
| 99 | 25a | XXXXX | XXXXX | ND | ND |
| 100 | 25b | XXXXX | XXXXX | XXXX | XXXX |
| | | | | X | X |
| 101 | 25c | XXXXX | XXXXX | XXXX | XXXX |
| | | | | X | X |
| 102 | 25d | XXXXX | XXXXX | ND | ND |
| 104 | 25f | XXXXX | XXXXX | ND | ND |
| 105 | 25g | XXXX | XXX | ND | ND |
| 106 | 25h | XXXXX | XXXXX | ND | ND |
| 107 | 25i | XXXXX | XXXXX | XXXX | XXXXX |
| 108 | 25j | XXXXX | XXXXX | ND | ND |
| 109 | 25k | XXXXX | XXXXX | ND | ND |
| 110 | 251 | XXXXX | XXXXX | ND | ND |
| 111 | 25m | XXXXX | XXXXX | ND | ND |
| 112 | 25n | XXXXX | XXXX | ND | ND |
| 113 | 26a | XXXXX | XXXX | ND | ND |
| 114 | 26b | XXXXX | XXXX | ND | ND |
| 115 | 27a | XXXXX | XXXXX | ND | ND |
| 116 | 25o | X | X | ND | ND |
| 117 | 25p | XXX | XX | ND | ND |
| 118 | 25q | XXXXX | XXXX | ND | ND |
| 119 | 25r | XXXXX | XXXXX | ND | ND |
| 120 | 25s | XXX | ND | ND | ND |
| 121 | 25t | X | X | ND | ND |
| 122 | 25u | XXXXX | XXXX | ND | ND |
| 123 | 25v | XXXXX | XXXXX | ND | ND |
| 124 | 25w | XXXXX | XXXXX | ND | ND |
| 125 | 25x | XXXXX | XXXXX | ND | ND |
| 126 | 25y | XXXXX | XXXX | ND | ND |
| 127 | 25z | XXXXX | XXXX | ND | ND |
| 128 | 25aa | XXXXX | XXX | ND | ND |
| 129 | 25bb | XXXXX | XXXXX | ND | ND |
| 130 | 25cc | XXXXX | XXXXX | ND | ND |
| 131 | 25dd | XXXXX | XXXXX | ND | ND |
| 132 | 25ee | XXXXX | XXXXX | ND | ND |
| 133 | 25ff | XXXXX | XXXXX | ND | ND |
| 134 | 25gg | XXXXX | XXXXX | ND | ND |
| 135 | 25hh | XXXXX | XXXXX | ND | ND |
| 136 | 25ii | XXXXX | XXXXX | ND | ND |
| 137 | 25jj | XXXXX | XXXXX | ND | ND |
| 138 | 25kk | XXXXX | XXXXX | ND | ND |
| 139 | 25ll | XXXXX | XXXX | ND | ND |
| 140 | 25mm | XXXXX | XXXXX | ND | ND |
| 141 | 25nn | XXXXX | XXXXX | ND | ND |
| 142 | 25oo | XXXXX | XXXXX | ND | ND |
| 143 | 25pp | XXXX | XXX | ND | ND |
| 144 | 25qq | XXXXX | XXXX | ND | ND |
| 145 | 25rr | XXXXX | XXX | ND | ND |
| 146 | 25ss | XXX | XX | ND | ND |
| 147 | 25tt | XXXXX | XXXXX | ND | ND |
| 148 | 25uu | XXXXX | XXXXX | ND | ND |
| 149 | 25vv | XXXXX | XXXXX | ND | ND |
| 150 | 25ww | XXXXX | XXXXX | ND | ND |
| 151 | 25xx | XXXXX | XXXXX | ND | ND |
| 152 | 25yy | XXXXX | XXXXX | ND | ND |
| 153 | 25zz | XXXXX | XXXXX | ND | ND |
| 154 | 25aaa | XXXXX | XXXXX | ND | ND |
| 155 | 25bbb | XXXXX | XXXXX | ND | ND |
| 156 | 25ccc | XXXXX | XXX | ND | ND |
| 157 | 25ddd | XXXX | XXXX | ND | ND |
| 158 | 25eee | XX | XX | ND | ND |
| 159 | 25fff | XXXX | XXXX | ND | ND |
| 160 | 25ggg | XXXX | XXXX | ND | ND |
| 161 | 25hhh | XXXX | XXX | ND | ND |
| 162 | 25iii | XXXX | XXXX | ND | ND |
| | | X | | | |
| 163 | 25jjj | X | X | ND | ND |
| 164 | 25kkk | XX | XX | ND | ND |
| 165 | 25lll | X | ND | ND | ND |
| 166 | 25mmm | ND | ND | ND | ND |
| 167 | 25nnn | XXXX | XXX | ND | ND |
| 168 | 25ooo | XXXX | XXX | ND | ND |
| 169 | 25ppp | XXXX | XXXX | ND | ND |
| 170 | 25qqq | XXXX | XXXX | ND | ND |
| | | X | X | | |
| 171 | 25rrr | XXXX | XXXX | ND | ND |
| | | X | X | | |
| 172 | 25sss | XXXX | XXXX | ND | ND |
| | | X | X | | |
| 173 | 25ttt | XXXX | XXXX | ND | ND |
| 174 | 25uuu | XXXX | XXXX | ND | ND |
| | | X | | | |
| 175 | 25vvv | XXXXX | XXXX | ND | ND |
| 176 | 25www | XXXXX | XXXXX | ND | ND |
| 177 | 25xxx | XXXXX | ND | ND | ND |
| 178 | 25yyy | XXXXX | ND | ND | ND |
| 179 | 25zzz | XXXXX | ND | ND | ND |
| 180 | 25aaaa | XXXXX | XXXXX | ND | ND |
| 181 | 25bbbb | XXXXX | ND | ND | ND |
| 182 | 25cccc | XXXXX | ND | ND | ND |
| 183 | 25dddd | XXX | ND | ND | ND |
| 184 | 25eeee | XXXX | XXXX | ND | ND |
| 185 | 25ffff | XXX | X | ND | ND |
| 186 | 25gggg | XXX | XX | ND | ND |
| 187 | 35 | XXXX | XXX | ND | ND |
| 188 | 39a | XXXXX | XXXXX | ND | ND |
| 189 | 39b | XXXX | XXXX | ND | ND |
| 190 | 42 | XXXXX | ND | ND | ND |
| 191 | 44 | XXXXX | ND | ND | ND |
| 192 | 47 | XXXXX | ND | ND | ND |
| 193 | 48 | XXXX | XXXX | ND | ND |
| | | X | X | | |
| 194 | 49 | XXXX | XXXX | ND | ND |
| | | X | X | | |
| 195 | 50 | XXXX | XXXX | ND | ND |
| 196 | 53a | X | X | ND | ND |
| 197 | 53b | XX | X | ND | ND |
| 198 | 53c | X | X | ND | ND |
| 199 | 53d | XX | X | ND | ND |
| 200 | 53e | X | X | ND | ND |
| 201 | 53f | X | X | ND | ND |
| 202 | 55a | X | X | ND | ND |
| 203 | 55b | XXXX | XXX | ND | ND |
| 204 | 56 | XXXX | XXX | ND | ND |
| 205 | 59a | XXXX | XXXX | ND | ND |
| 206 | 59b | XXXX | XXX | ND | ND |
| 207 | 59c | XXXX | XXX | ND | ND |
| 208 | 60a | XXXX | XXXX | ND | ND |
| | | X | | | |
| 209 | 60b | XXXX | XXX | ND | ND |
| | | X | | | |
| 210 | 62 | X | X | ND | ND |

ND = not determined.

| | | | | 151-500 | 501-1000 | >1000 |
|---|---|---|---|---|---|---|
| | EC₅₀ | 1-50 nM | 51-150 nM | nM | nM | nM |
| Table 1 | | XXXXX | XXXX | XXX | XX | X |
| Key | | | | | | |

### In Vivo Assessments:

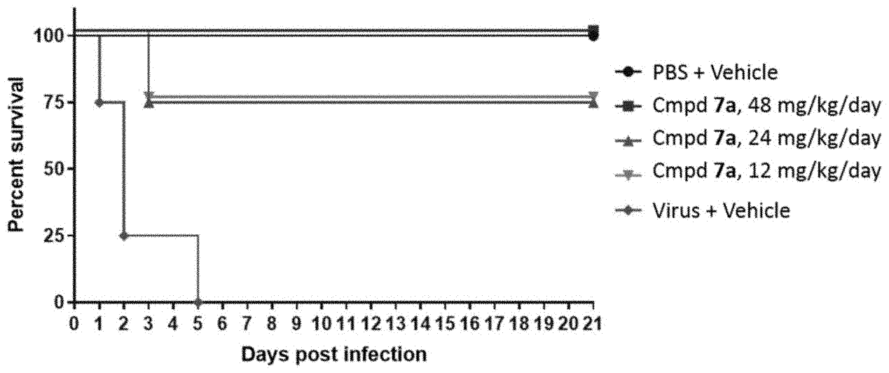

See FIG. 1. Fourteen (14) day in vivo efficacy assessment of Example 55, compound 7a in 6-8 week old BALB/C mice (groups of 8). Study was a prophylactic assessment: compound was given to mice 2 h prior to viral exposure with VEEV TrD (10×LD50, subcutaneous challenge). Compound was delivered via IP injection. Per day dose was divided into two doses, given 12 h apart for 8 days (e.g. 12 mg/kg/day was given as 6 mg/kg BID). Virus+vehicle mice all died without compound treatment by day 5. Mice without virus all survived (control, 100% survival). 48 mg/kg/day group had a 100% survival rate. Mice in the 24 mg/kg/day and 12 mg/kg/day groups had 75% survival rates. Survival plot lines are offset for clarity.

TABLE 2
Fourteen (14) day in vivo efficacy assessment of Example 55, compound 7a in
BALB/C mice. Study contained prophylactic and treatment arms: compound was given to mice 2
h prior, or 24 h after, or 48 h after viral exposure with VEEV TrD (10xLD50, subcutaneous
challenge). Compound was delivered via IP injection at 12.5 mg/kg BID, 12 h apart for 8 days.
All treated mice showed 100% survival rates even when treated 48 h after viral exposure with
VEEV TrD. All untreated mice died.

| | | | | Virus | day 14 |
|---|---|---|---|---|---|
| Mouse | Dose of | Dosing Timeline Relative to | Viral | Challenge | % |
| Group | compound 7a | VEEV TrD Challenge | challenge? | Timeline | survival |
| A | none | none | no | none | 100% |
| B | none | none | yes | day 0 | 0% |
| C | 12.5 mg/kg | 2 h prior to viral challenge, day 0 | yes | day 0 | 100% |
| | BID for 8 | | | | |
| | days | | | | |
| D | 12.5 mg/kg | 24 h after viral challenge, day 1 | yes | day 0 | 100% |
| | BID for 8 | | | | |
| | days | | | | |
| E | 12.5 mg/kg | 48 h after viral challenge, day 2 | yes | day 0 | 100% |
| | BID for 8 | | | | |
| | days | | | | |

TABLE 3
Twenty-one (21) day prophylactic in vivo efficacy assessment of Example 55,
compound 7a in C57BL/6 mice infected by EEEV FL93-939 (10-15 mice/group). Study was a
prophylactic assessment: compound was given to mice 2 h prior to viral exposure with EEEV
(subcutaneous challenge dose: 10^{4.3} CCID₅₀/0.2 mL). Compound was delivered via IP injection
Per day dose was divided into two doses, given 12 h apart for 8 days (e.g. 12 mg/kg/day was
given as 6 mg/kg BID). All untreated mice died. Compound 7a showed a dose-dependent,
protective response against EEEV.

| Mouse | | | |
|---|---|---|---|
| Group | Dose of compound 7a | EEEV challenge? | % survival on day 21 |
| A | none | no | 100% |
| B | none | yes | 0% |
| C | 50 mg/kg/day | yes | 70% |
| D | 25 mg/kg/day | yes | 60% |
| E | 12 mg/kg/day | yes | 30% |

What is claimed is:

1. A compound having a structure as shown in Formula I and Formula II: 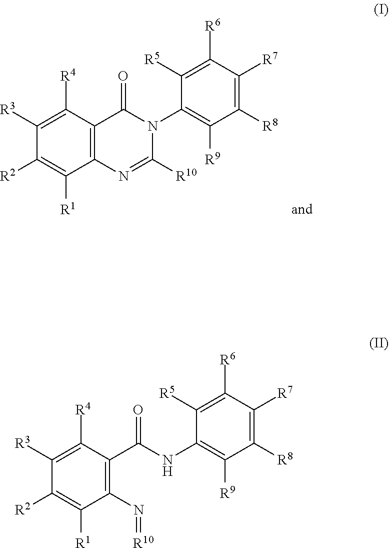
wherein:
R¹, R², R³ and R⁴ are the same or different and are independently selected from the group consisting of hydrogen, halogen, alkyl, C₃-C₈-cycloalkyl, halogen-substituted alkyl, alkynyl, alkoxy, halogen-substituted alkoxy, nitro, cyano, aryl, alkyl-substituted aryl, alkoxy-substituted aryl, halogen-substituted aryl, saturated or unsaturated 4- to 6-membered heterocyclyl, sulfonyl, and sulfonamidyl,
provided that not all of R¹, R², R³ and R⁴ are simultaneously hydrogen, and
provided that at least one of R¹, R², R³ and R⁴ is an electron-withdrawing group;
R⁵, R⁶, R⁷, R⁸, and R⁹ are the same or different and are independently selected from the group consisting of hydrogen, halogen, alkyl, unsubstituted or substituted C₃-C₈-cycloalkyl, halogen-substituted alkyl, alkoxy, halogen-substituted alkoxy, azido, nitro, and cyano;
R¹⁰ is selected from the group consisting of aliphatic amino, aliphatic alkylamino, 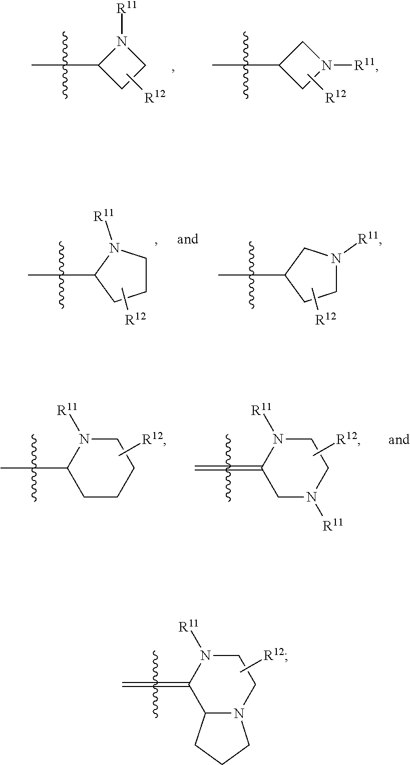
and
wherein:
R¹¹ is selected from the group consisting of hydrogen, halogen, alkyl, halogen-substituted alkyl, alkoxy, halogen-substituted alkoxy, unsubstituted or substituted C₃-C₈-cyclic ether, and unsubstituted or C-substituted or N-substituted saturated or unsaturated nitrogen C₄-C₈ heterocyclyl;
R¹² is selected from the group consisting of hydrogen, halogen, alkyl, halogen-substituted alkyl, alkoxy, and halogen-substituted alkoxy;
and salts thereof.

2. The compound of claim 1, wherein R¹⁰ is selected from the group consisting of

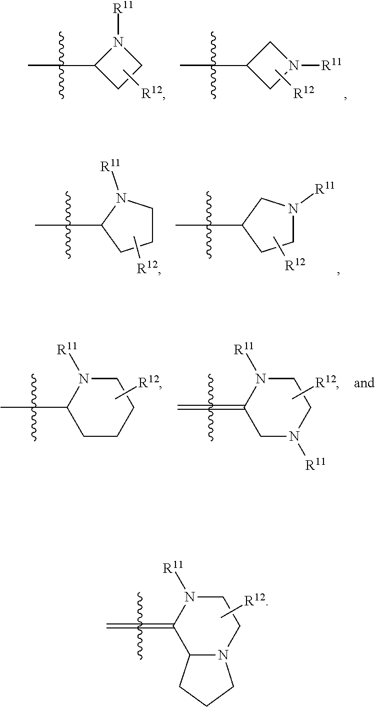

3. The compound of claim 2, wherein R¹¹ is selected from the group consisting of hydrogen, halogen, and alkyl.

4. The compound of claim 2, wherein R¹² is selected from the group consisting of hydrogen, halogen, and alkyl.

5. The compound of claim 2, wherein R¹¹ is selected from the group consisting of unsubstituted or substituted C₃-C₆-cyclic ether, and unsubstituted or C-substituted or N-substituted C₄-C₆-saturated nitrogen heterocyclyl.

6. The compound of claim 1, wherein R³ is an electron-withdrawing group selected from the group consisting of fluoro, nitro, cyano, sulfonyl, and sulfonamide.

7. The compound of claim 6, wherein R¹ is not hydrogen.

8. The compound of claim 6, wherein at least two of R¹, R², R³, and R⁴ are not hydrogen.

9. The compound of claim 1, wherein R¹ is not hydrogen.

10. The compound of claim 1, wherein at least two of R¹, R², R³, and R⁴ are not hydrogen.

11. The compound of claim 1, having a structure as shown in Formula I.

12. The compound of claim 11, wherein R¹ is selected from the group consisting of halogen, alkyl, halogen-substituted alkyl, C₃-C₈-cycloalkyl, 3-pyrimadinyl, and 4-pyrimadinyl.

13. The compound of claim 11, wherein R¹⁰ is selected from the group consisting of

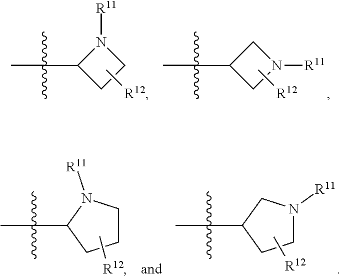

14. The compound of claim 1, having a structure as shown in Formula II.

15. The compound of claim 14, wherein R¹⁰ is selected from the group consisting of

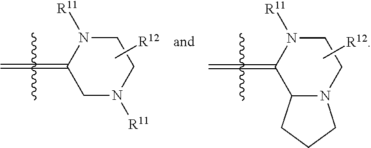

16. The compound of claim 1, having a structure as shown in Formula I, and wherein R¹⁰ is

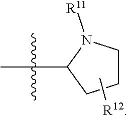

17. The compound of claim 16, wherein R¹¹ is not hydrogen.

18. The compound of claim 16, wherein R¹² is not hydrogen.

19. The compound of claim 16, wherein R³ is an electron-withdrawing group selected from the group consisting of fluoro, nitro, cyano, sulfonyl, and sulfonamide.

20. A pharmaceutical composition for inhibiting a viral infection, the composition comprising a viral inhibitory-effective amount of one or more compounds as recited in claim 1.

21. A method of inhibiting a viral infection, the method comprising administering to a subject a viral inhibitory-effective amount of a compound as recited in claim 1.
